SEQUENCE LISTING (621 sequences)

SEQ ID NO: 1 - protein, 133 aa, Synthetic construct
APTSSSTKKTQLQLEHLLLDLQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Synthetic construct
APTSSSTKKTQLQLEHLLLDLQMILNGINNYKNPKLTRMLTFKFYMPKKATELKHLQCLEEELKPLEEALNLAQSKNFHLRPRDLISNINVIVLELKGSETTFMCEYADETATIVEFLNRWITFCQSIISTLT

SEQ ID NO: 3 - protein, 133 aa, Synthetic construct
APTSSSTKKTQLQLEHLLLDLQMILNGINNYKNPKLTRMLTFKFYMPKKATELKHLQCLEEELKPLEEELNLAQSKNFHLRPRDLISNINVIVLELKGSETTFMCEYADETATIVEFLNRWITFCQSIISTLT

SEQ ID NO: 4 - protein, 133 aa, Synthetic construct
APTSSSTKKTQLQLEHLLLDLQMILNGINNYKNPKLTRMLTFKFYMPKKATELKHLQCLEEELKPLEEFLNLAQSKNFHLRPRDLISNINVIVLELKGSETTFMCEYADETATIVEFLNRWITFCQSIISTLT

SEQ ID NO: 5 - protein, 133 aa, Synthetic construct
APTSSSTKKTQLQLEHLLLDLQMILNGINNYKNPKLTRMLTFKFYMPKKATELKHLQCLEEELKPLEEGLNLAQSKNFHLRPRDLISNINVIVLELKGSETTFMCEYADETATIVEFLNRWITFCQSIISTLT

SEQ ID NO: 6 - protein, 133 aa, Synthetic construct
APTSSSTKKTQLQLEHLLLDLQMILNGINNYKNPKLTRMLTFKFYMPKKATELKHLQCLEEELKPLEEHLNLAQSKNFHLRPRDLISNINVIVLELKGSETTFMCEYADETATIVEFLNRWITFCQSIISTLT

SEQ ID NO: 7 - protein, 133 aa, Synthetic construct
APTSSSTKKTQLQLEHLLLDLQMILNGINNYKNPKLTRMLTFKFYMPKKATELKHLQCLEEELKPLEEILNLAQSKNFHLRPRDLISNINVIVLELKGSETTFMCEYADETATIVEFLNRWITFCQSIISTLT

SEQ ID NO: 8 - protein, 133 aa, Synthetic construct
APTSSSTKKTQLQLEHLLLDLQMILNGINNYKNPKLTRMLTFKFYMPKKATELKHLQCLEEELKPLEEKLNLAQSKNFHLRPRDLISNINVIVLELKGSETTFMCEYADETATIVEFLNRWITFCQSIISTLT

SEQ ID NO: 9 - protein, 133 aa, Synthetic construct
APTSSSTKKTQLQLEHLLLDLQMILNGINNYKNPKLTRMLTFKFYMPKKATELKHLQCLEEELKPLEELLNLAQSKNFHLRPRDLISNINVIVLELKGSETTFMCEYADETATIVEFLNRWITFCQSIISTLT

SEQ ID NO: 10 - protein, 133 aa, Synthetic construct
APTSSSTKKTQLQLEHLLLDLQMILNGINNYKNPKLTRMLTFKFYMPKKATELKHLQCLEEELKPLEEMLNLAQSKNFHLRPRDLISNINVIVLELKGSETTFMCEYADETATIVEFLNRWITFCQSIISTLT

SEQ ID NO: 11 - protein, 133 aa, Synthetic construct
APTSSSTKKTQLQLEHLLLDLQMILNGINNYKNPKLTRMLTFKFYMPKKATELKHLQCLEEELKPLEEQLNLAQSKNFHLRPRDLISNINVIVLELKGSETTFMCEYADETATIVEFLNRWITFCQSIISTLT

SEQ ID NO: 12 - protein, 133 aa, Synthetic construct
APTSSSTKKTQLQLEHLLLDLQMILNGINNYKNPKLTRMLTFKFYMPKKATELKHLQCLEEELKPLEESLNLAQSKNFHLRPRDLISNINVIVLELKGSETTFMCEYADETATIVEFLNRWITFCQSIISTLT

SEQ ID NO: 13 - protein, 133 aa, Synthetic construct
APTSSSTKKTQLQLEHLLLDLQMILNGINNYKNPKLTRMLTFKFYMPKKATELKHLQCLEEELKPLEETLNLAQSKNFHLRPRDLISNINVIVLELKGSETTFMCEYADETATIVEFLNRWITFCQSIISTLT

SEQ ID NO: 14 - protein, 133 aa, Synthetic construct
APTSSSTKKTQLQLEHLLLDLQMILNGINNYKNPKLTRMLTFKFYMPKKATELKHLQCLEEELKPLEEWLNLAQSKNFHLRPRDLISNINVIVLELKGSETTFMCEYADETATIVEFLNRWITFCQSIISTLT

SEQ ID NO: 15 - protein, 133 aa, Synthetic construct
APTSSSTKKTQLQLEHLLLDLQMILNGINNYKNPKLTRMLTFKFYMPKKATELKHLQCLEEELKPLEEYLNLAQSKNFHLRPRDLISNINVIVLELKGSETTFMCEYADETATIVEFLNRWITFCQSIISTLT

SEQ ID NO: 16 - protein, 133 aa, Synthetic construct
APTSSSTKKTQLQLEHLLLDLQMILNGINNYKNPKLTRMLTKKKYMPKKATELKHLQCLEEELKPLEEVLNLAQSKNFHLRPRDLISNINVIVLELKGSETTFMCEYADETATIVEFLNRWITFCQSIISTLT

SEQ ID NO: 17 - protein, 133 aa, Synthetic construct
APTSSSTKKTQLQLEHLLLDLQMILNGINNYKNPKLTRMLTFKKRMPKKATELKHLQCLEEELKPLEEVLNLAQSKNFHLRPRDLISNINVIVLELKGSETTFMCEYADETATIVEFLNRWITFCQSIISTLT

SEQ ID NO: 18 - protein, 133 aa, Synthetic construct
APTSSSTKKTQLQLEHLLLDLQMILNGINNYKNPKLTRMLTKKFYMPKKATELKHLQCLEEELKPLEERLNLAQSKNFHLRPRDLISNINVIVLELKGSETTFMCEYADETATIVEFLNRWITFCQSIISTLT

SEQ ID NO: 19 - protein, 133 aa, Synthetic construct
APTSSSTKKTQLQLEHLLLDLQMILNGINNYKNPKLTRMLTFKFRMPKKATELKHLQCLEEELKPLEERLNLAQSKNFHLRPRDLISNINVIVLELKGSETTFMCEYADETATIVEFLNRWITFCQSIISTLT

SEQ ID NO: 20 - protein, 133 aa, Synthetic construct
APTSSSTKKTQLQLEHLLLDLQMILNGINNYKNPKLTRMLTKKKRMPKKATELKHLQCLEEELKPLEEVLNLAQSKNFHLRPRDLISNINVIVLELKGSETTFMCEYADETATIVEFLNRWITFCQSIISTLT

SEQ ID NO: 21 - protein, 133 aa, Synthetic construct
APTSSSTKKTQLQLEHLLLDLQMILNGINNYKNPKLTAMLTKKFRMPKKATELKHLQCLEEELKPLEEVLNLAQSKNFHLRPRDLISNINVIVLELKGSETTFMCEYADETATIVEFLNRWITFCQSIISTLT

SEQ ID NO: 22 - protein, 133 aa, Synthetic construct
APTSSSTKKTQLQLEHLLLDLQMILNGINNYKNPKLTEMLTKKFRMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 133 aa, Synthetic construct
APTSSSTKKTQLQLEHLLLDLQMILNGINNYKNPKLTRMLTKEFRMPKKATELKHLQCLEEELKPLEEVLNLAQSKNFHLRPRDLISNINVIVLELKGSETTFMCEYADETATIVEFLNRWITFCQSIISTLT

SEQ ID NO: 24 - protein, 133 aa, Synthetic construct
APTSSSTKKTQLQLEHLLLDLQMILNGINNYKNPKLTRMLTKTFRMPKKATELKHLQCLEEELKPLEEVLNLAQSKNFHLRPRDLISNINVIVLELKGSETTFMCEYADETATIVEFLNRWITFCQSIISTLT

SEQ ID NO: 25 - protein, 133 aa, Synthetic construct
APTSSSTKKTQLQLEHLLLDLQMILNGINNYKNPKLTRMLTKKFRMPKKATELKHLQCLEEALKPLEEVLNLAQSKNFHLRPRDLISNINVIVLELKGSETTFMCEYADETATIVEFLNRWITFCQSIISTLT

SEQ ID NO: 26 - protein, 133 aa, Synthetic construct
APTSSSTKKTQLQLEHLLLDLQMILNGINNYKNPKLTRMLTKKFRMPKKATELKHLQCLEEELKRLEEVLNLAQSKNFHLRPRDLISNINVIVLELKGSETTFMCEYADETATIVEFLNRWITFCQSIISTLT

SEQ ID NO: 27 - protein, 133 aa, Synthetic construct
APTSSSTKKTQLQLEHLLLDLQMILNGINNYKNPKLTRMLTKKFRMPKKATELKHLQCLEEELKSLEEVLNLAQSKNFHLRPRDLISNINVIVLELKGSETTFMCEYADETATIVEFLNRWITFCQSIISTLT

SEQ ID NO: 28 - protein, 133 aa, Synthetic construct
APTSSSTKKTQLQLEHLLLDLQMILNGINNYKNPKLTRMLTKKFRMPKKATELKHLQCLEEELKPLEEALNLAQSKNFHLRPRDLISNINVIVLELKGSETTFMCEYADETATIVEFLNRWITFCQSIISTLT

SEQ ID NO: 29 - protein, 133 aa, Synthetic construct
APTSSSTKKTQLQLEHLLLDLQMILNGINNYKNPKLTRMLTKKFRMPKKATELKHLQCLEEELKPLEEDLNLAQSKNFHLRPRDLISNINVIVLELKGSETTFMCEYADETATIVEFLNRWITFCQSIISTLT

SEQ ID NO: 30 - protein, 133 aa, Synthetic construct
APTSSSTKKTQLQLEHLLLDLQMILNGINNYKNPKLTRMLTKKFRMPKKATELKHLQCLEEELKPLEERLNLAQSKNFHLRPRDLISNINVIVLELKGSETTFMCEYADETATIVEFLNRWITFCQSIISTLT

SEQ ID NO: 31 - protein, 133 aa, Synthetic construct
APTSSSTKKTQLQLEHLLLALQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 32 - protein, 133 aa, Synthetic construct
APTSSSTKKTQLQLEHLLLNLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 33 - protein, 133 aa, Synthetic construct
APTSSSTKKTQLQLEHLLLKLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 34 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 35 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISGINVIVLELKGSETTFMCEYADETATIVEFLNRWITFCQSIISTLT

SEQ ID NO: 36 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISHINVIVLELKGSETTFMCEYADETATIVEFLNRWITFCQSIISTLT

SEQ ID NO: 37 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISKINVIVLELKGSETTFMCEYADETATIVEFLNRWITFCQSIISTLT

SEQ ID NO: 38 - protein, 133 aa, Synthetic construct
APTSSSTKKTQLQLEHLLLALQMILNGINNYKNPKLTRMLTFKFYMPKKATELKHLQCLEEELKPLEEVLNLAQSKNFHLRPRALISNINVIVLELKGSETTFMCEYADETATIVEFLNRWITFCQSIISTLT

SEQ ID NO: 39 - protein, 133 aa, Synthetic construct
APTSSSTKKTQLQLAHLLLALQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 40 - protein, 133 aa, Synthetic construct
APTSSSTKKTQLQLEHLLLALQMILNGINNYKNPKLTRMLTFKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 41 - protein, 133 aa, Synthetic construct
APTSSSTKKTQLQLEHLLLALQMILNGINNYKNPKLTRMLTFKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 42 - protein, 133 aa, Synthetic construct
APTSSSTKKTQLQLEHLLLALQMILNGINNYKNPKLTRMLTFKFYMPKKATELKHLQCLEEELKPLEEVLNLAQSKNFHLRPRDLIANINVIVLELKGSETTFMCEYADETATIVEFLNRWITFCQSIISTLT

SEQ ID NO: 43 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRALISAINVIVLELKGSETTFMCEYADETATIVEFLNRWITFCQSIISTLT

SEQ ID NO: 44 - protein, 133 aa, Synthetic construct
APTSSSTKKTQLQLAHLLLDLQMILNGINNYKNPKLTRMLTFKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 45 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLIAAINVIVLELKGSETTFMCEYADETATIVEFLNRWITFCQSIISTLT

SEQ ID NO: 46 - protein, 133 aa, Synthetic construct
APTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 47 - protein, 133 aa, Synthetic construct
APTSSSTKKTQLQLEHLLLDLQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 48 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 49 - protein, 133 aa, Synthetic construct
APTSSSTKKTQLQLEHLLLDLQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 50 - protein, 133 aa, Synthetic construct
APTSSSTKKTQLQLEHLLLDLQMILNGINNYKNPKLTRMLTRKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 51 - protein, 133 aa, Synthetic construct
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 52 - protein, 133 aa, Synthetic construct
APTSSSTKKTQLQLEHLLLDLQMILNGINNYKNPKLTRMLTDKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 53 - protein, 133 aa, Synthetic construct
APTSSSTKKTQLQLEHLLLDLQMILNGINNYKNPKLTRMLTHKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 54 - protein, 133 aa, Synthetic construct
APTSSSTKKTQLQLEHLLLDLQMILNGINNYKNPKLTRMLTFAFYMPKKATELKHLQCLEEELKPLEEVLNLAQSKNFHLRPRDLISNINVIVLELKGSETTFMCEYADETATIVEFLNRWITFCQSIISTLT

SEQ ID NO: 55 - protein, 133 aa, Synthetic construct
APTSSSTKKTQLQLEHLLLDLQMILNGINNYKNPKLTRMLTFEFYMPKKATELKHLQCLEEELKPLEEVLNLAQSKNFHLRPRDLISNINVIVLELKGSETTFMCEYADETATIVEFLNRWITFCQSIISTLT

SEQ ID NO: 56 - protein, 133 aa, Synthetic construct
APTSSSTKKTQLQLEHLLLDLQMILNGINNYKNPKLTRMLTFQFYMPKKATELKHLQCLEEELKPLEEVLNLAQSKNFHLRPRDLISNINVIVLELKGSETTFMCEYADETATIVEFLNRWITFCQSIISTLT

SEQ ID NO: 57 - protein, 133 aa, Synthetic construct
APTSSSTKKTQLQLEHLLLDLQMILNGINNYKNPKLTRMLTFKFAMPKKATELKHLQCLEEELKPLEEVLNLAQSKNFHLRPRDLISNINVIVLELKGSETTFMCEYADETATIVEFLNRWITFCQSIISTLT

SEQ ID NO: 58 - protein, 133 aa, Synthetic construct
APTSSSTKKTQLQLEHLLLDLQMILNGINNYKNPKLTRMLTFKFKMPKKATELKHLQCLEEELKPLEEVLNLAQSKNFHLRPRDLISNINVIVLELKGSETTFMCEYADETATIVEFLNRWITFCQSIISTLT

SEQ ID NO: 59 - protein, 133 aa, Synthetic construct
APTSSSTKKTQLQLEHLLLDLQMILNGINNYKNPKLTRMLTFKFSMPKKATELKHLQCLEEELKPLEEVLNLAQSKNFHLRPRDLISNINVIVLELKGSETTFMCEYADETATIVEFLNRWITFCQSIISTLT

SEQ ID NO: 60 - protein, 133 aa, Synthetic construct
APTSSSTKKTQLQLEHLLLDLQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFCQSIISTLT

SEQ ID NO: 61 - protein, 133 aa, Synthetic construct
APTSSSTKKTQLQLEHLLLDLQMILNGINNYKNPKLTRMLTFKFYMPKKATELKHLQCLEAELKPLEEVLNLAQSKNFHLRPRDLISNINVIVLELKGSETTFMCEYADETATIVEFLNRWITFCQSIISTLT

SEQ ID NO: 62 - protein, 133 aa, Synthetic construct
APTSSSTKKTQLQLEHLLLDLQMILNGINNYKNPKLTRMLTFKFYMPKKATELKHLQCLERELKPLEEVLNLAQSKNFHLRPRDLISNINVIVLELKGSETTFMCEYADETATIVEFLNRWITFCQSIISTLT

SEQ ID NO: 63 - protein, 133 aa, Synthetic construct
APTSSSTKKTQLQLEHLLLDLQMILNGINNYKNPKLTRMLTFKFYMPKKATELKHLQCLEKELKPLEEVLNLAQSKNFHLRPRDLISNINVIVLELKGSETTFMCEYADETATIVEFLNRWITFCQSIISTLT

SEQ ID NO: 64 - protein, 133 aa, Synthetic construct
APTSSSTKKTQLQLEHLLLDLQMILNGINNYKNPKLTRMLTFKFYMPKKATELKHLQCLEEALKPLEEVLNLAQSKNFHLRPRDLISNINVIVLELKGSETTFMCEYADETATIVEFLNRWITFCQSIISTLT

SEQ ID NO: 65 - protein, 133 aa, Synthetic construct
APTSSSTKKTQLQLEHLLLDLQMILNGINNYKNPKLTRMLTFKFYMPKKATELKHLQCLEERLKPLEEVLNLAQSKNFHLRPRDLISNINVIVLELKGSETTFMCEYADETATIVEFLNRWITFCQSIISTLT

SEQ ID NO: 66 - protein, 133 aa, Synthetic construct
APTSSSTKKTQLQLEHLLLDLQMILNGINNYKNPKLTRMLTFKFYMPKKATELKHLQCLEEKLKPLEEVLNLAQSKNFHLRPRDLISNINVIVLELKGSETTFMCEYADETATIVEFLNRWITFCQSIISTLT

SEQ ID NO: 67 - protein, 133 aa, Synthetic construct
APTSSSTKKTQLQLEHLLLDLQMILNGINNYKNPKLTRMLTFKFYMPKKATELKHLQCLEEYLKPLEEVLNLAQSKNFHLRPRDLISNINVIVLELKGSETTFMCEYADETATIVEFLNRWITFCQSIISTLT

SEQ ID NO: 68 - protein, 133 aa, Synthetic construct
APTSSSTKKTQLQLEHLLLDLQMILNGINNYKNPKLTRMLTFKFYMPKKATELKHLQCLEEELKPLEYVLNLAQSKNFHLRPRDLISNINVIVLELKGSETTFMCEYADETATIVEFLNRWITFCQSIISTLT

SEQ ID NO: 69 - protein, 133 aa, Synthetic construct
APTSSSTKKTQLQLEHLLLDLQMILNGINNYKNPKLTRMLTFKFYMPKKATELKHLQCLEEELKPLEAVLNLAQSKNFHLRPRDLISNINVIVLELKGSETTFMCEYADETATIVEFLNRWITFCQSIISTLT

SEQ ID NO: 70 - protein, 133 aa, Synthetic construct
APTSSSTKKTQLQLEHLLLDLQMILNGINNYKNPKLTRMLTFKFYMPKKATELKHLQCLEEELKPLEKVLNLAQSKNFHLRPRDLISNINVIVLELKGSETTFMCEYADETATIVEFLNRWITFCQSIISTLT

SEQ ID NO: 71 - protein, 133 aa, Synthetic construct
APTSSSTKKTQLQLEHLLLDLQMILNGINNYKNPKLTRMLTFKFYMPKKATELKHLQCLEEELKPLERVLNLAQSKNFHLRPRDLISNINVIVLELKGSETTFMCEYADETATIVEFLNRWITFCQSIISTLT

SEQ ID NO: 72 - protein, 133 aa, Synthetic construct
APTSSSTKKTQLQLEHLLLDLQMILNGINNYKNPKLTRMLTFKFYMPKKATELKHLQCLEEELKPLELVLNLAQSKNFHLRPRDLISNINVIVLELKGSETTFMCEYADETATIVEFLNRWITFCQSIISTLT

SEQ ID NO: 73 - protein, 133 aa, Synthetic construct
APTSSSTKKTQLQLEHLLLDLQMILNGINNYKNPKLTRMLTFKFYMPKKATELKHLQCLEEELKPLEEVLNYAQSKNFHLRPRDLISNINVIVLELKGSETTFMCEYADETATIVEFLNRWITFCQSIISTLT

SEQ ID NO: 74 - protein, 133 aa, Synthetic construct
APTSSSTKKTQLQLEHLLLDLQMILNGINNYKNPKLTRMLTFKFYMPKKATELKHLQCLEEELKPLEEVLNRAQSKNFHLRPRDLISNINVIVLELKGSETTFMCEYADETATIVEFLNRWITFCQSIISTLT

SEQ ID NO: 75 - protein, 133 aa, Synthetic construct
APTSSSTKKTQLQLEHLLLDLQMILNGINNYKNPKLTRMLTFKFYMPKKATELKHLQCLEEELKPLEEVLNAAQSKNFHLRPRDLISNINVIVLELKGSETTFMCEYADETATIVEFLNRWITFCQSIISTLT

SEQ ID NO: 76 - protein, 133 aa, Synthetic construct
APTSSSTKKTQLQLEHLLLDLQMILNGINNYKNPKLTRMLTFKFYMPKKATELKHLQCLEEELKPLEEVLNDAQSKNFHLRPRDLISNINVIVLELKGSETTFMCEYADETATIVEFLNRWITFCQSIISTLT

SEQ ID NO: 77 - protein, 133 aa, Synthetic construct
APTSSSTKKTQLQLEHLLLDLQMILNGINNYKNPKLTRMLTFKFYMPKKATELKHLQCLEEELKPLEEVLNHAQSKNFHLRPRDLISNINVIVLELKGSETTFMCEYADETATIVEFLNRWITFCQSIISTLT

SEQ ID NO: 78 - protein, 133 aa, Synthetic construct
APTSSSTKKTQLQLEHLLLDLQMILNGINNYKNPKLTRMLTFKFYMPKKATELKHLQCLEEELKPLEEVLNFAQSKNFHLRPRDLISNINVIVLELKGSETTFMCEYADETATIVEFLNRWITFCQSIISTLT

SEQ ID NO: 79 - protein, 133 aa, Synthetic construct
APTSSSTKKTQLQLEHLLLDLQMILNGINNYKNPKLTDMLTFKFYMPKKATELKHLQCLERELKPLEEVLNLAQSKNFHLRPRDLISNINVIVLELKGSETTFMCEYADETATIVEFLNRWITFCQSIISTLT

SEQ ID NO: 80 - protein, 133 aa, Synthetic construct
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLT

SEQ ID NO: 81 - protein, 133 aa, Synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 82 - protein, 133 aa, Synthetic construct
APTSSSTKKTQLQLAHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 83 - protein, 133 aa, Synthetic construct
APTSSSTKKTQLQLRHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 84 - protein, 133 aa, Synthetic construct
APTSSSTKKTQLQLKHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 85 - protein, 133 aa, Synthetic construct
APTSSSTKKTQLQLEALLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 86 - protein, 133 aa, Synthetic construct
APTSSSTKKTQLQLEYLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 87 - protein, 133 aa, Synthetic construct
APTSSSTKKTQLQLEELLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 88 - protein, 133 aa, Synthetic construct
APTSSSTKKTQLQLEHLLADLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 89 - protein, 133 aa, Synthetic construct
APTSSSTKKTQLQLEHLLLILQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 90 - protein, 133 aa, Synthetic construct
APTSSSTKKTQLQLEHLLLSLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 91 - protein, 133 aa, Synthetic construct
APTSSSTKKTQLQLEHLLLHLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 92 - protein, 133 aa, Synthetic construct
APTSSSTKKTQLQLEHLLLTLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 93 - protein, 133 aa, Synthetic construct
APTSSSTKKTQLQLEHLLLWLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 94 - protein, 133 aa, Synthetic construct
APTSSSTKKTQLQLEHLLLYLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 95 - protein, 133 aa, Synthetic construct
APTSSSTKKTQLQLEHLLLRLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 96 - protein, 133 aa, Synthetic construct
APTSSSTKKTQLQLEHLLLFLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 97 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLAPRDLISNINVIVLELKGSETTFMCEYADETATIVEFLNRWITFCQSIISTLT

SEQ ID NO: 98 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRALISNINVIVLELKGSETTFMCEYADETATIVEFLNRWITFCQSIISTLT

SEQ ID NO: 99 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 100 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 101 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLIANINVIVLELKGSETTFMCEYADETATIVEFLNRWITFCQSIISTLT

SEQ ID NO: 102 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISYINVIVLELKGSETTFMCEYADETATIVEFLNRWITFCQSIISTLT

SEQ ID NO: 103 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 104 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 105 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISEINVIVLELKGSETTFMCEYADETATIVEFLNRWITFCQSIISTLT

SEQ ID NO: 106 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISFINVIVLELKGSETTFMCEYADETATIVEFLNRWITFCQSIISTLT

SEQ ID NO: 107 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISIINVIVLELKGSETTFMCEYADETATIVEFLNRWITFCQSIISTLT

SEQ ID NO: 108 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVAVLELKGSETTFMCEYADETATIVEFLNRWITFCQSIISTLT

SEQ ID NO: 109 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVYVLELKGSETTFMCEYADETATIVEFLNRWITFCQSIISTLT

SEQ ID NO: 110 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVSVLELKGSETTFMCEYADETATIVEFLNRWITFCQSIISTLT

SEQ ID NO: 111 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVFVLELKGSETTFMCEYADETATIVEFLNRWITFCQSIISTLT

SEQ ID NO: 112 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVRVLELKGSETTFMCEYADETATIVEFLNRWITFCQSIISTLT

SEQ ID NO: 113 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 114 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVEVLELKGSETTFMCEYADETATIVEFLNRWITFCQSIISTLT

SEQ ID NO: 115 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 116 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLRLKGSETTFMCEYADETATIVEFLNRWITFCQSIISTLT

SEQ ID NO: 117 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLKLKGSETTFMCEYADETATIVEFLNRWITFCQSIISTLT

SEQ ID NO: 118 - protein, 133 aa, Synthetic construct
APTSSSTKKTQLQLEELLLYLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 119 - protein, 133 aa, Synthetic construct
APTSSSTKKTQLQLEALLLYLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 120 - protein, 133 aa, Synthetic construct
APTSSSTKKTQLQLEYLLLYLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 121 - protein, 133 aa, Synthetic construct
APTSSSTKKTQLQLEHLLLYLQMILNGINNYKNPKLTRMLTFKFYMPKKATELKHLQCLEEELKPLEEVLNLAQSKNFHLRPRDLISNINVAVLELKGSETTFMCEYADETATIVEFLNRWITFCQSIISTLT

SEQ ID NO: 122 - protein, 133 aa, Synthetic construct
APTSSSTKKTQLQLEHLLLYLQMILNGINNYKNPKLTRMLTFKFYMPKKATELKHLQCLEEELKPLEEVLNLAQSKNFHLRPRDLISNINVSVLELKGSETTFMCEYADETATIVEFLNRWITFCQSIISTLT

SEQ ID NO: 123 - protein, 133 aa, Synthetic construct
APTSSSTKKTQLQLEHLLLYLQMILNGINNYKNPKLTRMLTFKFYMPKKATELKHLQCLEEELKPLEEVLNLAQSKNFHLRPRDLISNINVRVLELKGSETTFMCEYADETATIVEFLNRWITFCQSIISTLT

SEQ ID NO: 124 - protein, 133 aa, Synthetic construct
APTSSSTKKTQLQLEHLLLYLQMILNGINNYKNPKLTRMLTFKFYMPKKATELKHLQCLEEELKPLEEVLNLAQSKNFHLRPRDLISNINVIVLRLKGSETTFMCEYADETATIVEFLNRWITFCQSIISTLT

SEQ ID NO: 125 - protein, 133 aa, Synthetic construct
APTSSSTKKTQLQLEHLLLYLQMILNGINNYKNPKLTRMLTFKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 126 - protein, 164 aa, Synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICT

SEQ ID NO: 127 - protein, 133 aa, Synthetic construct
APTSSSTKKTQLQLEHLLLALQMILNGINNYKNPKLTRMLTDKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 128 - protein, 133 aa, Synthetic construct
APTSSSTKKTQLQLEHLLLALQMILNGINNYKNPKLTRMLTRKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 129 - protein, 133 aa, Synthetic construct
APTSSSTKKTQLQLEHLLLALQMILNGINNYKNPKLTRMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 130 - protein, 133 aa, Synthetic construct
APTSSSTKKTQLQLEHLLLALQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 131 - protein, 133 aa, Synthetic construct
APTSSSTKKTQLQLEHLLLALQMILNGINNYKNPKLTRMLTHKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 132 - protein, 133 aa, Synthetic construct
APTSSSTKKTQLQLEHLLLALQMILNGINNYKNPKLTRMLTFKFRMPKKATELKHLQCLEEELKPLEEVLNLAQSKNFHLRPRDLISNINVIVLELKGSETTFMCEYADETATIVEFLNRWITFCQSIISTLT

SEQ ID NO: 133 - protein, 133 aa, Synthetic construct
APTSSSTKKTQLQLEHLLLALQMILNGINNYKNPKLTRMLTFKFKMPKKATELKHLQCLEEELKPLEEVLNLAQSKNFHLRPRDLISNINVIVLELKGSETTFMCEYADETATIVEFLNRWITFCQSIISTLT

SEQ ID NO: 134 - protein, 133 aa, Synthetic construct
APTSSSTKKTQLQLEHLLLALQMILNGINNYKNPKLTNMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 135 - protein, 133 aa, Synthetic construct
APTSSSTKKTQLQLEHLLLALQMILNGINNYKNPKLTGMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 136 - protein, 133 aa, Synthetic construct
APTSSSTKKTQLQLEHLLLALQMILNGINNYKNPKLTHMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 137 - protein, 133 aa, Synthetic construct
APTSSSTKKTQLQLEHLLLALQMILNGINNYKNPKLTIMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 138 - protein, 133 aa, Synthetic construct
APTSSSTKKTQLQLEHLLLALQMILNGINNYKNPKLTLMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 139 - protein, 133 aa, Synthetic construct
APTSSSTKKTQLQLEHLLLALQMILNGINNYKNPKLTMMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 140 - protein, 133 aa, Synthetic construct
APTSSSTKKTQLQLEHLLLALQMILNGINNYKNPKLTFMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 141 - protein, 133 aa, Synthetic construct
APTSSSTKKTQLQLEHLLLALQMILNGINNYKNPKLTPMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 142 - protein, 133 aa, Synthetic construct
APTSSSTKKTQLQLEHLLLALQMILNGINNYKNPKLTSMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 143 - protein, 133 aa, Synthetic construct
APTSSSTKKTQLQLEHLLLALQMILNGINNYKNPKLTTMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 144 - protein, 133 aa, Synthetic construct
APTSSSTKKTQLQLEHLLLALQMILNGINNYKNPKLTWMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 145 - protein, 133 aa, Synthetic construct
APTSSSTKKTQLQLEHLLLALQMILNGINNYKNPKLTYMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 146 - protein, 133 aa, Synthetic construct
APTSSSTKKTQLQLEHLLLALQMILNGINNYKNPKLTVMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 147 - protein, 133 aa, Synthetic construct
APTSSSTKKTQLQLEHLLLALQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 148 - protein, 133 aa, Synthetic construct
APTSSSTKKTQLQLEHLLLALQMILNGINNYKNPKLTQMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 149 - protein, 133 aa, Synthetic construct
APTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 150 - protein, 133 aa, Synthetic construct
APTSSSTKKTQLQLEHLLLALQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 151 - protein, 133 aa, Synthetic construct
APTSSSTKKTQLQLEHLLLALQMILNGINNYKNPKLTRMLTFEFYMPKKATELKHLQCLEEELKPLEEVLNLAQSKNFHLRPRDLISNINVIVLELKGSETTFMCEYADETATIVEFLNRWITFCQSIISTLT

SEQ ID NO: 152 - protein, 133 aa, Synthetic construct
APTSSSTKKTQLQLEHLLLALQMILNGINNYKNPKLTRMLTFKFYMPKKATELKHLQCLEAELKPLEEVLNLAQSKNFHLRPRDLISNINVIVLELKGSETTFMCEYADETATIVEFLNRWITFCQSIISTLT

SEQ ID NO: 153 - protein, 133 aa, Synthetic construct
APTSSSTKKTQLQLEHLLLALQMILNGINNYKNPKLTRMLTFKFYMPKKATELKHLQCLEEALKPLEEVLNLAQSKNFHLRPRDLISNINVIVLELKGSETTFMCEYADETATIVEFLNRWITFCQSIISTLT

SEQ ID NO: 154 - protein, 133 aa, Synthetic construct
APTSSSTKKTQLQLEHLLLALQMILNGINNYKNPKLTRMLTFKFYMPKKATELKHLQCLEEYLKPLEEVLNLAQSKNFHLRPRDLISNINVIVLELKGSETTFMCEYADETATIVEFLNRWITFCQSIISTLT

SEQ ID NO: 155 - protein, 133 aa, Synthetic construct
APTSSSTKKTQLQLEHLLLALQMILNGINNYKNPKLTRMLTFKFYMPKKATELKHLQCLEEELKPLEEVLNDAQSKNFHLRPRDLISNINVIVLELKGSETTFMCEYADETATIVEFLNRWITFCQSIISTLT

SEQ ID NO: 156 - protein, 133 aa, Synthetic construct
APTSSSTKKTQLQLEHLLLALQMILNGINNYKNPKLTRMLTFKFYMPKKATELKHLQCLEEELKPLEEVLNHAQSKNFHLRPRDLISNINVIVLELKGSETTFMCEYADETATIVEFLNRWITFCQSIISTLT

SEQ ID NO: 157 - protein, 133 aa, Synthetic construct
APTSSSTKKTQLQLEHLLLALQMILNGINNYKNPKLTRMLTFKFYMPKKATELKHLQCLEEELKPLEEVLNRAQSKNFHLRPRDLISNINVIVLELKGSETTFMCEYADETATIVEFLNRWITFCQSIISTLT

SEQ ID NO: 158 - protein, 133 aa, Synthetic construct
APTSSSTKKTQLQLEHLLLDLQMILNGINNYKNPKLTRMLTDKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 159 - protein, 133 aa, Synthetic construct
APTSSSTKKTQLQLEHLLLDLQMILNGINNYKNPKLTRMLTRKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 160 - protein, 133 aa, Synthetic construct
APTSSSTKKTQLQLEHLLLDLQMILNGINNYKNPKLTRMLTHKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 161 - protein, 133 aa, Synthetic construct
APTSSSTKKTQLQLEHLLLDLQMILNGINNYKNPKLTRMLTAKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 162 - protein, 133 aa, Synthetic construct
APTSSSTKKTQLQLEHLLLDLQMILNGINNYKNPKLTRMLTFEFYMPKKATELKHLQCLEEELKPLEEVLNLAQSKNFHLRPRDLISNINVDVLELKGSETTFMCEYADETATIVEFLNRWITFCQSIISTLT

SEQ ID NO: 163 - protein, 133 aa, Synthetic construct
APTSSSTKKTQLQLEHLLLDLQMILNGINNYKNPKLTRMLTFKFRMPKKATELKHLQCLEEELKPLEEVLNLAQSKNFHLRPRDLISNINVDVLELKGSETTFMCEYADETATIVEFLNRWITFCQSIISTLT

SEQ ID NO: 164 - protein, 133 aa, Synthetic construct
APTSSSTKKTQLQLEHLLLDLQMILNGINNYKNPKLTRMLTFKFKMPKKATELKHLQCLEEELKPLEEVLNLAQSKNFHLRPRDLISNINVDVLELKGSETTFMCEYADETATIVEFLNRWITFCQSIISTLT

SEQ ID NO: 165 - protein, 133 aa, Synthetic construct
APTSSSTKKTQLQLEHLLLDLQMILNGINNYKNPKLTRMLTFKFYMPKKATELKHLQCLEEALKPLEEVLNLAQSKNFHLRPRDLISNINVDVLELKGSETTFMCEYADETATIVEFLNRWITFCQSIISTLT

SEQ ID NO: 166 - protein, 133 aa, Synthetic construct
APTSSSTKKTQLQLEHLLLDLQMILNGINNYKNPKLTRMLTFKFYMPKKATELKHLQCLEEYLKPLEEVLNLAQSKNFHLRPRDLISNINVDVLELKGSETTFMCEYADETATIVEFLNRWITFCQSIISTLT

SEQ ID NO: 167 - protein, 133 aa, Synthetic construct
APTSSSTKKTQLQLEHLLLDLQMILNGINNYKNPKLTRMLTFKFYMPKKATELKHLQCLEEELKPLEEVLNDAQSKNFHLRPRDLISNINVDVLELKGSETTFMCEYADETATIVEFLNRWITFCQSIISTLT

SEQ ID NO: 168 - protein, 133 aa, Synthetic construct
APTSSSTKKTQLQLEHLLLDLQMILNGINNYKNPKLTRMLTFKFYMPKKATELKHLQCLEEELKPLEEVLNHAQSKNFHLRPRDLISNINVDVLELKGSETTFMCEYADETATIVEFLNRWITFCQSIISTLT

SEQ ID NO: 169 - protein, 133 aa, Synthetic construct
APTSSSTKKTQLQLEHLLLDLQMILNGINNYKNPKLTRMLTFKFYMPKKATELKHLQCLEEELKPLEEVLNRAQSKNFHLRPRDLISNINVDVLELKGSETTFMCEYADETATIVEFLNRWITFCQSIISTLT

SEQ ID NO: 170 - protein, 133 aa, Synthetic construct
APTSSSTKKTQLQLEHLLLDLQMILNGINNYKNPKLTDMLTFKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 171 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 172 - protein, 133 aa, Synthetic construct
APTSSSTKKTQLQLEHLLLDLQMILNGINNYKNPKLTQMLTFKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 173 - protein, 133 aa, Synthetic construct
APTSSSTKKTQLQLEHLLLDLQMILNGINNYKNPKLTAMLTFKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 174 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 175 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRRLISNINVIVLELKGSETTFMCEYADETATIVEFLNRWITFCQSIISTLT

SEQ ID NO: 176 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 177 - protein, 133 aa, Synthetic construct
APTSSSTKKTQLQLEHLLLALQMILNGINNYKNPKLTRMLTAKFRMPKKATELKHLQCLEEELKPLEEVLNLAQSKNFHLRPRDLISNINVIVLELKGSETTFMCEYADETATIVEFLNRWITFCQSIISTLT

SEQ ID NO: 178 - protein, 133 aa, Synthetic construct
APTSSSTKKTQLQLEHLLLALQMILNGINNYKNPKLTRMLTHKFRMPKKATELKHLQCLEEELKPLEEVLNLAQSKNFHLRPRDLISNINVIVLELKGSETTFMCEYADETATIVEFLNRWITFCQSIISTLT

SEQ ID NO: 179 - protein, 133 aa, Synthetic construct
APTSSSTKKTQLQLEHLLLALQMILNGINNYKNPKLTDMLTFKFYMPKKATELKHLQCLERELKPLEEVLNLAQSKNFHLRPRDLISNINVIVLELKGSETTFMCEYADETATIVEFLNRWITFCQSIISTLT

SEQ ID NO: 180 - protein, 133 aa, Synthetic construct
APTSSSTKKTQLQLEHLLLALQMILNGINNYKNPKLTEMLTFKFYMPKKATELKHLQCLERELKPLEEVLNLAQSKNFHLRPRDLISNINVIVLELKGSETTFMCEYADETATIVEFLNRWITFCQSIISTLT

SEQ ID NO: 181 - protein, 133 aa, Synthetic construct
APTSSSTKKTQLQLEHLLLALQMILNGINNYKNPKLTQMLTFKFYMPKKATELKHLQCLERELKPLEEVLNLAQSKNFHLRPRDLISNINVIVLELKGSETTFMCEYADETATIVEFLNRWITFCQSIISTLT

SEQ ID NO: 182 - protein, 133 aa, Synthetic construct
APTSSSTKKTQLQLEHLLLALQMILNGINNYKNPKLTAMLTFKFYMPKKATELKHLQCLERELKPLEEVLNLAQSKNFHLRPRDLISNINVIVLELKGSETTFMCEYADETATIVEFLNRWITFCQSIISTLT

SEQ ID NO: 183 - protein, 133 aa, Synthetic construct
APTSSSTKKTQLQLEHLLLALQMILNGINNYKNPKLTAMLTFKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 184 - protein, 133 aa, Synthetic construct
APTSSSTKKTQLQLEHLLLALQMILNGINNYKNPKLTDMLTFKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 185 - protein, 133 aa, Synthetic construct
APTSSSTKKTQLQLEHLLLALQMILNGINNYKNPKLTEMLTFKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 186 - protein, 133 aa, Synthetic construct
APTSSSTKKTQLQLEHLLLALQMILNGINNYKNPKLTQMLTFKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 187 - protein, 133 aa, Synthetic construct
APTSSSTKKTQLQLEHLLLALQMILNGINNYKNPKLTRMLTRKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 188 - protein, 133 aa, Synthetic construct
APTSSSTKKTQLQLEHLLLALQMILNGINNYKNPKLTRMLTAKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 189 - protein, 133 aa, Synthetic construct
APTSSSTKKTQLQLEHLLLALQMILNGINNYKNPKLTRMLTDKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 190 - protein, 133 aa, Synthetic construct
APTSSSTKKTQLQLEHLLLALQMILNGINNYKNPKLTRMLTHKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 191 - protein, 133 aa, Synthetic construct
APTSSSTKKTQLQLEHLLLALQMILNGINNYKNPKLTRMLTKKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 192 - protein, 133 aa, Synthetic construct
APTSSSTKKTQLQLEHLLLALQMILNGINNYKNPKLTRMLTFAFYMPKKATELKHLQCLEEELKPLEEVLNLAQSKNFHLRPRDLISNINVIVLALKGSETTFMCEYADETATIVEFLNRWITFCQSIISTLT

SEQ ID NO: 193 - protein, 133 aa, Synthetic construct
APTSSSTKKTQLQLEHLLLALQMILNGINNYKNPKLTRMLTFEFYMPKKATELKHLQCLEEELKPLEEVLNLAQSKNFHLRPRDLISNINVIVLALKGSETTFMCEYADETATIVEFLNRWITFCQSIISTLT

SEQ ID NO: 194 - protein, 133 aa, Synthetic construct
APTSSSTKKTQLQLEHLLLALQMILNGINNYKNPKLTRMLTFQFYMPKKATELKHLQCLEEELKPLEEVLNLAQSKNFHLRPRDLISNINVIVLALKGSETTFMCEYADETATIVEFLNRWITFCQSIISTLT

SEQ ID NO: 195 - protein, 133 aa, Synthetic construct
APTSSSTKKTQLQLEHLLLALQMILNGINNYKNPKLTRMLTFKFAMPKKATELKHLQCLEEELKPLEEVLNLAQSKNFHLRPRDLISNINVIVLALKGSETTFMCEYADETATIVEFLNRWITFCQSIISTLT

SEQ ID NO: 196 - protein, 133 aa, Synthetic construct
APTSSSTKKTQLQLEHLLLALQMILNGINNYKNPKLTRMLTFKFKMPKKATELKHLQCLEEELKPLEEVLNLAQSKNFHLRPRDLISNINVIVLALKGSETTFMCEYADETATIVEFLNRWITFCQSIISTLT

SEQ ID NO: 197 - protein, 133 aa, Synthetic construct
APTSSSTKKTQLQLEHLLLALQMILNGINNYKNPKLTRMLTFKFSMPKKATELKHLQCLEEELKPLEEVLNLAQSKNFHLRPRDLISNINVIVLALKGSETTFMCEYADETATIVEFLNRWITFCQSIISTLT

SEQ ID NO: 198 - protein, 133 aa, Synthetic construct
APTSSSTKKTQLQLEHLLLALQMILNGINNYKNPKLTRMLTFKFRMPKKATELKHLQCLEEELKPLEEVLNLAQSKNFHLRPRDLISNINVIVLALKGSETTFMCEYADETATIVEFLNRWITFCQSIISTLT

SEQ ID NO: 199 - protein, 133 aa, Synthetic construct
APTSSSTKKTQLQLEHLLLALQMILNGINNYKNPKLTRMLTFKFYMPKKATELKHLQCLEAELKPLEEVLNLAQSKNFHLRPRDLISNINVIVLALKGSETTFMCEYADETATIVEFLNRWITFCQSIISTLT

SEQ ID NO: 200 - protein, 133 aa, Synthetic construct
APTSSSTKKTQLQLEHLLLALQMILNGINNYKNPKLTRMLTFKFYMPKKATELKHLQCLEEALKPLEEVLNLAQSKNFHLRPRDLISNINVIVLALKGSETTFMCEYADETATIVEFLNRWITFCQSIISTLT

SEQ ID NO: 201 - protein, 133 aa, Synthetic construct
APTSSSTKKTQLQLEHLLLALQMILNGINNYKNPKLTRMLTFKFYMPKKATELKHLQCLEERLKPLEEVLNLAQSKNFHLRPRDLISNINVIVLALKGSETTFMCEYADETATIVEFLNRWITFCQSIISTLT

SEQ ID NO: 202 - protein, 133 aa, Synthetic construct
APTSSSTKKTQLQLEHLLLALQMILNGINNYKNPKLTRMLTFKFYMPKKATELKHLQCLEEKLKPLEEVLNLAQSKNFHLRPRDLISNINVIVLALKGSETTFMCEYADETATIVEFLNRWITFCQSIISTLT

SEQ ID NO: 203 - protein, 133 aa, Synthetic construct
APTSSSTKKTQLQLEHLLLALQMILNGINNYKNPKLTRMLTFKFYMPKKATELKHLQCLEEYLKPLEEVLNLAQSKNFHLRPRDLISNINVIVLALKGSETTFMCEYADETATIVEFLNRWITFCQSIISTLT

SEQ ID NO: 204 - protein, 133 aa, Synthetic construct
APTSSSTKKTQLQLEHLLLALQMILNGINNYKNPKLTRMLTFKFYMPKKATELKHLQCLEEELKPLEYVLNLAQSKNFHLRPRDLISNINVIVLALKGSETTFMCEYADETATIVEFLNRWITFCQSIISTLT

SEQ ID NO: 205 - protein, 133 aa, Synthetic construct
APTSSSTKKTQLQLEHLLLALQMILNGINNYKNPKLTRMLTFKFYMPKKATELKHLQCLEEELKPLEAVLNLAQSKNFHLRPRDLISNINVIVLALKGSETTFMCEYADETATIVEFLNRWITFCQSIISTLT

SEQ ID NO: 206 - protein, 133 aa, Synthetic construct
APTSSSTKKTQLQLEHLLLALQMILNGINNYKNPKLTRMLTFKFYMPKKATELKHLQCLEEELKPLELVLNLAQSKNFHLRPRDLISNINVIVLALKGSETTFMCEYADETATIVEFLNRWITFCQSIISTLT

SEQ ID NO: 207 - protein, 133 aa, Synthetic construct
APTSSSTKKTQLQLEHLLLALQMILNGINNYKNPKLTRMLTFKFYMPKKATELKHLQCLEEELKPLEEVLNYAQSKNFHLRPRDLISNINVIVLALKGSETTFMCEYADETATIVEFLNRWITFCQSIISTLT

SEQ ID NO: 208 - protein, 133 aa, Synthetic construct
APTSSSTKKTQLQLEHLLLALQMILNGINNYKNPKLTRMLTFKFYMPKKATELKHLQCLEEELKPLEEVLNRAQSKNFHLRPRDLISNINVIVLALKGSETTFMCEYADETATIVEFLNRWITFCQSIISTLT

SEQ ID NO: 209 - protein, 133 aa, Synthetic construct
APTSSSTKKTQLQLEHLLLALQMILNGINNYKNPKLTRMLTFKFYMPKKATELKHLQCLEEELKPLEEVLNAAQSKNFHLRPRDLISNINVIVLALKGSETTFMCEYADETATIVEFLNRWITFCQSIISTLT

SEQ ID NO: 210 - protein, 133 aa, Synthetic construct
APTSSSTKKTQLQLEHLLLALQMILNGINNYKNPKLTRMLTFKFYMPKKATELKHLQCLEEELKPLEEVLNDAQSKNFHLRPRDLISNINVIVLALKGSETTFMCEYADETATIVEFLNRWITFCQSIISTLT

SEQ ID NO: 211 - protein, 133 aa, Synthetic construct
APTSSSTKKTQLQLEHLLLALQMILNGINNYKNPKLTRMLTFKFYMPKKATELKHLQCLEEELKPLEEVLNHAQSKNFHLRPRDLISNINVIVLALKGSETTFMCEYADETATIVEFLNRWITFCQSIISTLT

SEQ ID NO: 212 - protein, 133 aa, Synthetic construct
APTSSSTKKTQLQLEHLLLALQMILNGINNYKNPKLTRMLTFKFYMPKKATELKHLQCLEEELKPLEEVLNFAQSKNFHLRPRDLISNINVIVLALKGSETTFMCEYADETATIVEFLNRWITFCQSIISTLT

SEQ ID NO: 213 - protein, 133 aa, Synthetic construct
APTSSSTKKTQLQLEHLLLALQMILNGINNYKNPKLTRMLTKKFRMPKKATELKHLQCLEEELKPLEEVLNLAQSKNFHLRPRDLIANINVIVLELKGSETTFMCEYADETATIVEFLNRWITFCQSIISTLT

SEQ ID NO: 214 - protein, 133 aa, Synthetic construct
APTSSSTKKTQLQLEHLLLALQMILNGINNYKNPKLTRMLTKKFRMPKKATELKHLQCLEEELKPLEEVLNLAQSKNFHLRPRDLISNINVIVLALKGSETTFMCEYADETATIVEFLNRWITFCQSIISTLT

SEQ ID NO: 215 - protein, 133 aa, Synthetic construct
APTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 216 - protein, 133 aa, Synthetic construct
APASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 217 - protein, 133 aa, Synthetic construct
APASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 218 - protein, 130 aa, Synthetic construct
SSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 219 - protein, 130 aa, Synthetic construct
SSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 220 - protein, 133 aa, Synthetic construct
APTSSSTKKTQLQLEHLLLDLAMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 221 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWIAFCQSIISTLT

SEQ ID NO: 222 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIASTLT

SEQ ID NO: 223 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIIATLT

SEQ ID NO: 224 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCASIISTLT

SEQ ID NO: 225 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCDSIISTLT

SEQ ID NO: 226 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCVSIISTLT

SEQ ID NO: 227 - protein, 133 aa, Synthetic construct
APTSSSTKKTQLQLEHLLLDLAMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIIATLT

SEQ ID NO: 228 - protein, 133 aa, Synthetic construct
APTSSSTKKTQLQLEHLLLDLQMILNGINNYKNPKLTRMLTKKFRMPKKATELKHLQCLEEELKPLEEVLNLAQSKNFHLRPRDLISNINVIVLELKGSETTFMCEYADETATIVEFLNRWITFCDSIISTLT

SEQ ID NO: 229 - protein, 133 aa, Synthetic construct
APTSSSTKKTQLQLEHLLLALQMILNGINNYKNPKLTRMLTFKFYMPKKATELKHLQCLEEELKPLEEVLNLAQSKNFHLRPRDLISNINVIVLALKGSETTFMCEYADETATIVEFLNRWITFCDSIISTLT

SEQ ID NO: 230 - protein, 133 aa, Synthetic construct
APTSSSTKKTQLQLEHLLLALQMILNGINNYKNPKLTRMLTFKFYMPKKATELKHLQCLERELKPLEEVLNLAQSKNFHLRPRDLISNINVIVLELKGSETTFMCEYADETATIVEFLNRWITFCQSIISTLT

SEQ ID NO: 231 - protein, 133 aa, Synthetic construct
APTSSSTKKTQLQLEHLLLALQMILNGINNYKNPKLTRMLTFKFYMPKKATELKHLQCLENELKPLEEVLNLAQSKNFHLRPRDLISNINVIVLELKGSETTFMCEYADETATIVEFLNRWITFCQSIISTLT

SEQ ID NO: 232 - protein, 133 aa, Synthetic construct
APTSSSTKKTQLQLEHLLLALQMILNGINNYKNPKLTRMLTFKFYMPKKATELKHLQCLEDELKPLEEVLNLAQSKNFHLRPRDLISNINVIVLELKGSETTFMCEYADETATIVEFLNRWITFCQSIISTLT

SEQ ID NO: 233 - protein, 133 aa, Synthetic construct
APTSSSTKKTQLQLEHLLLALQMILNGINNYKNPKLTRMLTFKFYMPKKATELKHLQCLEQELKPLEEVLNLAQSKNFHLRPRDLISNINVIVLELKGSETTFMCEYADETATIVEFLNRWITFCQSIISTLT

SEQ ID NO: 234 - protein, 133 aa, Synthetic construct
APTSSSTKKTQLQLEHLLLALQMILNGINNYKNPKLTRMLTFKFYMPKKATELKHLQCLEGELKPLEEVLNLAQSKNFHLRPRDLISNINVIVLELKGSETTFMCEYADETATIVEFLNRWITFCQSIISTLT

SEQ ID NO: 235 - protein, 133 aa, Synthetic construct
APTSSSTKKTQLQLEHLLLALQMILNGINNYKNPKLTRMLTFKFYMPKKATELKHLQCLEHELKPLEEVLNLAQSKNFHLRPRDLISNINVIVLELKGSETTFMCEYADETATIVEFLNRWITFCQSIISTLT

SEQ ID NO: 236 - protein, 133 aa, Synthetic construct
APTSSSTKKTQLQLEHLLLALQMILNGINNYKNPKLTRMLTFKFYMPKKATELKHLQCLEIELKPLEEVLNLAQSKNFHLRPRDLISNINVIVLELKGSETTFMCEYADETATIVEFLNRWITFCQSIISTLT

SEQ ID NO: 237 - protein, 133 aa, Synthetic construct
APTSSSTKKTQLQLEHLLLALQMILNGINNYKNPKLTRMLTFKFYMPKKATELKHLQCLELELKPLEEVLNLAQSKNFHLRPRDLISNINVIVLELKGSETTFMCEYADETATIVEFLNRWITFCQSIISTLT

SEQ ID NO: 238 - protein, 133 aa, Synthetic construct
APTSSSTKKTQLQLEHLLLALQMILNGINNYKNPKLTRMLTFKFYMPKKATELKHLQCLEKELKPLEEVLNLAQSKNFHLRPRDLISNINVIVLELKGSETTFMCEYADETATIVEFLNRWITFCQSIISTLT

SEQ ID NO: 239 - protein, 133 aa, Synthetic construct
APTSSSTKKTQLQLEHLLLALQMILNGINNYKNPKLTRMLTFKFYMPKKATELKHLQCLEMELKPLEEVLNLAQSKNFHLRPRDLISNINVIVLELKGSETTFMCEYADETATIVEFLNRWITFCQSIISTLT

SEQ ID NO: 240 - protein, 133 aa, Synthetic construct
APTSSSTKKTQLQLEHLLLALQMILNGINNYKNPKLTRMLTFKFYMPKKATELKHLQCLEFELKPLEEVLNLAQSKNFHLRPRDLISNINVIVLELKGSETTFMCEYADETATIVEFLNRWITFCQSIISTLT

SEQ ID NO: 241 - protein, 133 aa, Synthetic construct
APTSSSTKKTQLQLEHLLLALQMILNGINNYKNPKLTRMLTFKFYMPKKATELKHLQCLEPELKPLEEVLNLAQSKNFHLRPRDLISNINVIVLELKGSETTFMCEYADETATIVEFLNRWITFCQSIISTLT

SEQ ID NO: 242 - protein, 133 aa, Synthetic construct
APTSSSTKKTQLQLEHLLLALQMILNGINNYKNPKLTRMLTFKFYMPKKATELKHLQCLESELKPLEEVLNLAQSKNFHLRPRDLISNINVIVLELKGSETTFMCEYADETATIVEFLNRWITFCQSIISTLT

SEQ ID NO: 243 - protein, 133 aa, Synthetic construct
APTSSSTKKTQLQLEHLLLALQMILNGINNYKNPKLTRMLTFKFYMPKKATELKHLQCLETELKPLEEVLNLAQSKNFHLRPRDLISNINVIVLELKGSETTFMCEYADETATIVEFLNRWITFCQSIISTLT

SEQ ID NO: 244 - protein, 133 aa, Synthetic construct
APTSSSTKKTQLQLEHLLLALQMILNGINNYKNPKLTRMLTFKFYMPKKATELKHLQCLEWELKPLEEVLNLAQSKNFHLRPRDLISNINVIVLELKGSETTFMCEYADETATIVEFLNRWITFCQSIISTLT

SEQ ID NO: 245 - protein, 133 aa, Synthetic construct
APTSSSTKKTQLQLEHLLLALQMILNGINNYKNPKLTRMLTFKFYMPKKATELKHLQCLEYELKPLEEVLNLAQSKNFHLRPRDLISNINVIVLELKGSETTFMCEYADETATIVEFLNRWITFCQSIISTLT

SEQ ID NO: 246 - protein, 133 aa, Synthetic construct
APTSSSTKKTQLQLEHLLLALQMILNGINNYKNPKLTRMLTFKFYMPKKATELKHLQCLEVELKPLEEVLNLAQSKNFHLRPRDLISNINVIVLELKGSETTFMCEYADETATIVEFLNRWITFCQSIISTLT

SEQ ID NO: 247 - protein, 133 aa, Synthetic construct
APTSSSTKKTQLQLEHLLLALQMILNGINNYKNPKLTRMLTNKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 248 - protein, 133 aa, Synthetic construct
APTSSSTKKTQLQLEHLLLALQMILNGINNYKNPKLTRMLTQKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 249 - protein, 133 aa, Synthetic construct
APTSSSTKKTQLQLEHLLLALQMILNGINNYKNPKLTRMLTEKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 250 - protein, 133 aa, Synthetic construct
APTSSSTKKTQLQLEHLLLALQMILNGINNYKNPKLTRMLTGKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 251 - protein, 133 aa, Synthetic construct
APTSSSTKKTQLQLEHLLLALQMILNGINNYKNPKLTRMLTIKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 252 - protein, 133 aa, Synthetic construct
APTSSSTKKTQLQLEHLLLALQMILNGINNYKNPKLTRMLTLKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 253 - protein, 133 aa, Synthetic construct
APTSSSTKKTQLQLEHLLLALQMILNGINNYKNPKLTRMLTMKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 254 - protein, 133 aa, Synthetic construct
APTSSSTKKTQLQLEHLLLALQMILNGINNYKNPKLTRMLTPKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 255 - protein, 133 aa, Synthetic construct
APTSSSTKKTQLQLEHLLLALQMILNGINNYKNPKLTRMLTSKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 256 - protein, 133 aa, Synthetic construct
APTSSSTKKTQLQLEHLLLALQMILNGINNYKNPKLTRMLTTKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 257 - protein, 133 aa, Synthetic construct
APTSSSTKKTQLQLEHLLLALQMILNGINNYKNPKLTRMLTWKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 258 - protein, 133 aa, Synthetic construct
APTSSSTKKTQLQLEHLLLALQMILNGINNYKNPKLTRMLTYKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 259 - protein, 133 aa, Synthetic construct
APTSSSTKKTQLQLEHLLLALQMILNGINNYKNPKLTRMLTVKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 260 - protein, 133 aa, Synthetic construct
APTSSSTKKTQLQLEHLLLALQMILNGINNYKNPKLTRMLTFKFAMPKKATELKHLQCLEEELKPLEEVLNLAQSKNFHLRPRDLISNINVIVLELKGSETTFMCEYADETATIVEFLNRWITFCQSIISTLT

SEQ ID NO: 261 - protein, 133 aa, Synthetic construct
APTSSSTKKTQLQLEHLLLALQMILNGINNYKNPKLTRMLTFKFNMPKKATELKHLQCLEEELKPLEEVLNLAQSKNFHLRPRDLISNINVIVLELKGSETTFMCEYADETATIVEFLNRWITFCQSIISTLT

SEQ ID NO: 262 - protein, 133 aa, Synthetic construct
APTSSSTKKTQLQLEHLLLALQMILNGINNYKNPKLTRMLTFKFDMPKKATELKHLQCLEEELKPLEEVLNLAQSKNFHLRPRDLISNINVIVLELKGSETTFMCEYADETATIVEFLNRWITFCQSIISTLT

SEQ ID NO: 263 - protein, 133 aa, Synthetic construct
APTSSSTKKTQLQLEHLLLALQMILNGINNYKNPKLTRMLTFKFQMPKKATELKHLQCLEEELKPLEEVLNLAQSKNFHLRPRDLISNINVIVLELKGSETTFMCEYADETATIVEFLNRWITFCQSIISTLT

SEQ ID NO: 264 - protein, 133 aa, Synthetic construct
APTSSSTKKTQLQLEHLLLALQMILNGINNYKNPKLTRMLTFKFEMPKKATELKHLQCLEEELKPLEEVLNLAQSKNFHLRPRDLISNINVIVLELKGSETTFMCEYADETATIVEFLNRWITFCQSIISTLT

SEQ ID NO: 265 - protein, 133 aa, Synthetic construct
APTSSSTKKTQLQLEHLLLALQMILNGINNYKNPKLTRMLTFKFGMPKKATELKHLQCLEEELKPLEEVLNLAQSKNFHLRPRDLISNINVIVLELKGSETTFMCEYADETATIVEFLNRWITFCQSIISTLT

SEQ ID NO: 266 - protein, 133 aa, Synthetic construct
APTSSSTKKTQLQLEHLLLALQMILNGINNYKNPKLTRMLTFKFHMPKKATELKHLQCLEEELKPLEEVLNLAQSKNFHLRPRDLISNINVIVLELKGSETTFMCEYADETATIVEFLNRWITFCQSIISTLT

SEQ ID NO: 267 - protein, 133 aa, Synthetic construct
APTSSSTKKTQLQLEHLLLALQMILNGINNYKNPKLTRMLTFKFIMPKKATELKHLQCLEEELKPLEEVLNLAQSKNFHLRPRDLISNINVIVLELKGSETTFMCEYADETATIVEFLNRWITFCQSIISTLT

SEQ ID NO: 268 - protein, 133 aa, Synthetic construct
APTSSSTKKTQLQLEHLLLALQMILNGINNYKNPKLTRMLTFKFLMPKKATELKHLQCLEEELKPLEEVLNLAQSKNFHLRPRDLISNINVIVLELKGSETTFMCEYADETATIVEFLNRWITFCQSIISTLT

SEQ ID NO: 269 - protein, 133 aa, Synthetic construct
APTSSSTKKTQLQLEHLLLALQMILNGINNYKNPKLTRMLTFKFMMPKKATELKHLQCLEEELKPLEEVLNLAQSKNFHLRPRDLISNINVIVLELKGSETTFMCEYADETATIVEFLNRWITFCQSIISTLT

SEQ ID NO: 270 - protein, 133 aa, Synthetic construct
APTSSSTKKTQLQLEHLLLALQMILNGINNYKNPKLTRMLTFKFFMPKKATELKHLQCLEEELKPLEEVLNLAQSKNFHLRPRDLISNINVIVLELKGSETTFMCEYADETATIVEFLNRWITFCQSIISTLT

SEQ ID NO: 271 - protein, 133 aa, Synthetic construct
APTSSSTKKTQLQLEHLLLALQMILNGINNYKNPKLTRMLTFKFPMPKKATELKHLQCLEEELKPLEEVLNLAQSKNFHLRPRDLISNINVIVLELKGSETTFMCEYADETATIVEFLNRWITFCQSIISTLT

SEQ ID NO: 272 - protein, 133 aa, Synthetic construct
APTSSSTKKTQLQLEHLLLALQMILNGINNYKNPKLTRMLTFKFSMPKKATELKHLQCLEEELKPLEEVLNLAQSKNFHLRPRDLISNINVIVLELKGSETTFMCEYADETATIVEFLNRWITFCQSIISTLT

SEQ ID NO: 273 - protein, 133 aa, Synthetic construct
APTSSSTKKTQLQLEHLLLALQMILNGINNYKNPKLTRMLTFKFTMPKKATELKHLQCLEEELKPLEEVLNLAQSKNFHLRPRDLISNINVIVLELKGSETTFMCEYADETATIVEFLNRWITFCQSIISTLT

SEQ ID NO: 274 - protein, 133 aa, Synthetic construct
APTSSSTKKTQLQLEHLLLALQMILNGINNYKNPKLTRMLTFKFWMPKKATELKHLQCLEEELKPLEEVLNLAQSKNFHLRPRDLISNINVIVLELKGSETTFMCEYADETATIVEFLNRWITFCQSIISTLT

SEQ ID NO: 275 - protein, 133 aa, Synthetic construct
APTSSSTKKTQLQLEHLLLALQMILNGINNYKNPKLTRMLTFKFVMPKKATELKHLQCLEEELKPLEEVLNLAQSKNFHLRPRDLISNINVIVLELKGSETTFMCEYADETATIVEFLNRWITFCQSIISTLT

SEQ ID NO: 276 - protein, 133 aa, Synthetic construct
APTSSSTKKTQLQLEHLLLDLQMILNGINNYKNPKLTRMLTNKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 277 - protein, 133 aa, Synthetic construct
APTSSSTKKTQLQLEHLLLDLQMILNGINNYKNPKLTRMLTQKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 278 - protein, 133 aa, Synthetic construct
APTSSSTKKTQLQLEHLLLDLQMILNGINNYKNPKLTRMLTEKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 279 - protein, 133 aa, Synthetic construct
APTSSSTKKTQLQLEHLLLDLQMILNGINNYKNPKLTRMLTGKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 280 - protein, 133 aa, Synthetic construct
APTSSSTKKTQLQLEHLLLDLQMILNGINNYKNPKLTRMLTIKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 281 - protein, 133 aa, Synthetic construct
APTSSSTKKTQLQLEHLLLDLQMILNGINNYKNPKLTRMLTLKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 282 - protein, 133 aa, Synthetic construct
APTSSSTKKTQLQLEHLLLDLQMILNGINNYKNPKLTRMLTKKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 283 - protein, 133 aa, Synthetic construct
APTSSSTKKTQLQLEHLLLDLQMILNGINNYKNPKLTRMLTMKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 284 - protein, 133 aa, Synthetic construct
APTSSSTKKTQLQLEHLLLDLQMILNGINNYKNPKLTRMLTPKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 285 - protein, 133 aa, Synthetic construct
APTSSSTKKTQLQLEHLLLDLQMILNGINNYKNPKLTRMLTSKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 286 - protein, 133 aa, Synthetic construct
APTSSSTKKTQLQLEHLLLDLQMILNGINNYKNPKLTRMLTTKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 287 - protein, 133 aa, Synthetic construct
APTSSSTKKTQLQLEHLLLDLQMILNGINNYKNPKLTRMLTWKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 288 - protein, 133 aa, Synthetic construct
APTSSSTKKTQLQLEHLLLDLQMILNGINNYKNPKLTRMLTYKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 289 - protein, 133 aa, Synthetic construct
APTSSSTKKTQLQLEHLLLDLQMILNGINNYKNPKLTRMLTVKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 290 - protein, 133 aa, Synthetic construct
APTSSSTKKTQLQLEHLLLDLQMILNGINNYKNPKLTRMLTFKFAMPKKATELKHLQCLEEELKPLEEVLNLAQSKNFHLRPRDLISNINVDVLELKGSETTFMCEYADETATIVEFLNRWITFCQSIISTLT

SEQ ID NO: 291 - protein, 133 aa, Synthetic construct
APTSSSTKKTQLQLEHLLLDLQMILNGINNYKNPKLTRMLTFKFNMPKKATELKHLQCLEEELKPLEEVLNLAQSKNFHLRPRDLISNINVDVLELKGSETTFMCEYADETATIVEFLNRWITFCQSIISTLT

SEQ ID NO: 292 - protein, 133 aa, Synthetic construct
APTSSSTKKTQLQLEHLLLDLQMILNGINNYKNPKLTRMLTFKFDMPKKATELKHLQCLEEELKPLEEVLNLAQSKNFHLRPRDLISNINVDVLELKGSETTFMCEYADETATIVEFLNRWITFCQSIISTLT

SEQ ID NO: 293 - protein, 133 aa, Synthetic construct
APTSSSTKKTQLQLEHLLLDLQMILNGINNYKNPKLTRMLTFKFQMPKKATELKHLQCLEEELKPLEEVLNLAQSKNFHLRPRDLISNINVDVLELKGSETTFMCEYADETATIVEFLNRWITFCQSIISTLT

SEQ ID NO: 294 - protein, 133 aa, Synthetic construct
APTSSSTKKTQLQLEHLLLDLQMILNGINNYKNPKLTRMLTFKFEMPKKATELKHLQCLEEELKPLEEVLNLAQSKNFHLRPRDLISNINVDVLELKGSETTFMCEYADETATIVEFLNRWITFCQSIISTLT

SEQ ID NO: 295 - protein, 133 aa, Synthetic construct
APTSSSTKKTQLQLEHLLLDLQMILNGINNYKNPKLTRMLTFKFGMPKKATELKHLQCLEEELKPLEEVLNLAQSKNFHLRPRDLISNINVDVLELKGSETTFMCEYADETATIVEFLNRWITFCQSIISTLT

SEQ ID NO: 296 - protein, 133 aa, Synthetic construct
APTSSSTKKTQLQLEHLLLDLQMILNGINNYKNPKLTRMLTFKFHMPKKATELKHLQCLEEELKPLEEVLNLAQSKNFHLRPRDLISNINVDVLELKGSETTFMCEYADETATIVEFLNRWITFCQSIISTLT

SEQ ID NO: 297 - protein, 133 aa, Synthetic construct
APTSSSTKKTQLQLEHLLLDLQMILNGINNYKNPKLTRMLTFKFIMPKKATELKHLQCLEEELKPLEEVLNLAQSKNFHLRPRDLISNINVDVLELKGSETTFMCEYADETATIVEFLNRWITFCQSIISTLT

SEQ ID NO: 298 - protein, 133 aa, Synthetic construct
APTSSSTKKTQLQLEHLLLDLQMILNGINNYKNPKLTRMLTFKFLMPKKATELKHLQCLEEELKPLEEVLNLAQSKNFHLRPRDLISNINVDVLELKGSETTFMCEYADETATIVEFLNRWITFCQSIISTLT

SEQ ID NO: 299 - protein, 133 aa, Synthetic construct
APTSSSTKKTQLQLEHLLLDLQMILNGINNYKNPKLTRMLTFKFMMPKKATELKHLQCLEEELKPLEEVLNLAQSKNFHLRPRDLISNINVDVLELKGSETTFMCEYADETATIVEFLNRWITFCQSIISTLT

SEQ ID NO: 300 - protein, 133 aa, Synthetic construct
APTSSSTKKTQLQLEHLLLDLQMILNGINNYKNPKLTRMLTFKFFMPKKATELKHLQCLEEELKPLEEVLNLAQSKNFHLRPRDLISNINVDVLELKGSETTFMCEYADETATIVEFLNRWITFCQSIISTLT

SEQ ID NO: 301 - protein, 133 aa, Synthetic construct
APTSSSTKKTQLQLEHLLLDLQMILNGINNYKNPKLTRMLTFKFPMPKKATELKHLQCLEEELKPLEEVLNLAQSKNFHLRPRDLISNINVDVLELKGSETTFMCEYADETATIVEFLNRWITFCQSIISTLT

SEQ ID NO: 302 - protein, 133 aa, Synthetic construct
APTSSSTKKTQLQLEHLLLDLQMILNGINNYKNPKLTRMLTFKFSMPKKATELKHLQCLEEELKPLEEVLNLAQSKNFHLRPRDLISNINVDVLELKGSETTFMCEYADETATIVEFLNRWITFCQSIISTLT

SEQ ID NO: 303 - protein, 133 aa, Synthetic construct
APTSSSTKKTQLQLEHLLLDLQMILNGINNYKNPKLTRMLTFKFTMPKKATELKHLQCLEEELKPLEEVLNLAQSKNFHLRPRDLISNINVDVLELKGSETTFMCEYADETATIVEFLNRWITFCQSIISTLT

SEQ ID NO: 304 - protein, 133 aa, Synthetic construct
APTSSSTKKTQLQLEHLLLDLQMILNGINNYKNPKLTRMLTFKFWMPKKATELKHLQCLEEELKPLEEVLNLAQSKNFHLRPRDLISNINVDVLELKGSETTFMCEYADETATIVEFLNRWITFCQSIISTLT

SEQ ID NO: 305 - protein, 133 aa, Synthetic construct
APTSSSTKKTQLQLEHLLLDLQMILNGINNYKNPKLTRMLTFKFVMPKKATELKHLQCLEEELKPLEEVLNLAQSKNFHLRPRDLISNINVDVLELKGSETTFMCEYADETATIVEFLNRWITFCQSIISTLT

SEQ ID NO: 306 - protein, 133 aa, Synthetic construct
APTSSSTKKTQLQLEHLLLHLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 307 - protein, 133 aa, Synthetic construct
APTSSSTKKTQLQLEHLLLSLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 308 - protein, 133 aa, Synthetic construct
APTSSSTKKTQLQLEHLLLDLQMILNGINNYKNPKLTRMLTAKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 309 - protein, 133 aa, Synthetic construct
APTSSSTKKTQLQLEHLLLDLQMILNGINNYKNPKLTRMLTAKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 310 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRALISNINVIVLELKGSETTFMCEYADETATIVEFLNRWITFCQSIISTLT

SEQ ID NO: 311 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRNLISNINVIVLELKGSETTFMCEYADETATIVEFLNRWITFCQSIISTLT

SEQ ID NO: 312 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRQLISNINVIVLELKGSETTFMCEYADETATIVEFLNRWITFCQSIISTLT

SEQ ID NO: 313 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRELISNINVIVLELKGSETTFMCEYADETATIVEFLNRWITFCQSIISTLT

SEQ ID NO: 314 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRGLISNINVIVLELKGSETTFMCEYADETATIVEFLNRWITFCQSIISTLT

SEQ ID NO: 315 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 316 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRILISNINVIVLELKGSETTFMCEYADETATIVEFLNRWITFCQSIISTLT

SEQ ID NO: 317 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRLLISNINVIVLELKGSETTFMCEYADETATIVEFLNRWITFCQSIISTLT

SEQ ID NO: 318 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRMLISNINVIVLELKGSETTFMCEYADETATIVEFLNRWITFCQSIISTLT

SEQ ID NO: 319 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRFLISNINVIVLELKGSETTFMCEYADETATIVEFLNRWITFCQSIISTLT

SEQ ID NO: 320 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRPLISNINVIVLELKGSETTFMCEYADETATIVEFLNRWITFCQSIISTLT

SEQ ID NO: 321 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRSLISNINVIVLELKGSETTFMCEYADETATIVEFLNRWITFCQSIISTLT

SEQ ID NO: 322 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRTLISNINVIVLELKGSETTFMCEYADETATIVEFLNRWITFCQSIISTLT

SEQ ID NO: 323 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRWLISNINVIVLELKGSETTFMCEYADETATIVEFLNRWITFCQSIISTLT

SEQ ID NO: 324 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRYLISNINVIVLELKGSETTFMCEYADETATIVEFLNRWITFCQSIISTLT

SEQ ID NO: 325 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRVLISNINVIVLELKGSETTFMCEYADETATIVEFLNRWITFCQSIISTLT

SEQ ID NO: 326 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVAVLELKGSETTFMCEYADETATIVEFLNRWITFCQSIISTLT

SEQ ID NO: 327 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVRVLELKGSETTFMCEYADETATIVEFLNRWITFCQSIISTLT

SEQ ID NO: 328 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVNVLELKGSETTFMCEYADETATIVEFLNRWITFCQSIISTLT

SEQ ID NO: 329 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVQVLELKGSETTFMCEYADETATIVEFLNRWITFCQSIISTLT

SEQ ID NO: 330 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVEVLELKGSETTFMCEYADETATIVEFLNRWITFCQSIISTLT

SEQ ID NO: 331 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVGVLELKGSETTFMCEYADETATIVEFLNRWITFCQSIISTLT

SEQ ID NO: 332 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVHVLELKGSETTFMCEYADETATIVEFLNRWITFCQSIISTLT

SEQ ID NO: 333 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVLVLELKGSETTFMCEYADETATIVEFLNRWITFCQSIISTLT

SEQ ID NO: 334 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVKVLELKGSETTFMCEYADETATIVEFLNRWITFCQSIISTLT

SEQ ID NO: 335 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVMVLELKGSETTFMCEYADETATIVEFLNRWITFCQSIISTLT

SEQ ID NO: 336 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVFVLELKGSETTFMCEYADETATIVEFLNRWITFCQSIISTLT

SEQ ID NO: 337 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVPVLELKGSETTFMCEYADETATIVEFLNRWITFCQSIISTLT

SEQ ID NO: 338 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVSVLELKGSETTFMCEYADETATIVEFLNRWITFCQSIISTLT

SEQ ID NO: 339 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVTVLELKGSETTFMCEYADETATIVEFLNRWITFCQSIISTLT

SEQ ID NO: 340 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVWVLELKGSETTFMCEYADETATIVEFLNRWITFCQSIISTLT

SEQ ID NO: 341 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVYVLELKGSETTFMCEYADETATIVEFLNRWITFCQSIISTLT

SEQ ID NO: 342 - protein, 133 aa, Synthetic construct
APTSSSTKKTQLQLEHLLLDLQMILNGINNYKNPKLTEMLTFKFYMPKKATELKHLQCLEEELKPLEEVLNLAQSKNFHLRPRDLISNINVVVLELKGSETTFMCEYADETATIVEFLNRWITFCQSIISTLT

SEQ ID NO: 343 - protein, 133 aa, Synthetic construct
APTSSSTKKTQLQLEELLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 344 - protein, 133 aa, Synthetic construct
APTSSSTKKTQLQLEHLLLALQMILNGINNYKNPKLTKMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 345 - protein, 133 aa, Synthetic construct [sequence omitted]

SEQ ID NO: 346 - protein, 268 aa, Synthetic construct
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 347 - DNA, 804 bp, Synthetic construct
  source 1..804: mol_type = other DNA
ccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctc

SEQ ID NO: 348 - protein, 449 aa, Synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYASTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 349 - protein, 214 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 350 - protein, 456 aa, Synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYASTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 351 - protein, 218 aa, Synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 352 - protein, 347 aa, Synthetic construct
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 353 - protein, 525 aa, Synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 354 - protein, 251 aa, Synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 355 - protein, 6 aa, Synthetic construct
SGGGGS

SEQ ID NO: 356 - protein, 346 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTDTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 357 - protein, 352 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGGGSDTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 358 - protein, 580 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTEVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 586 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGGGSEVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 580 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 361 - protein, 586 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 362 - protein, 346 aa, Synthetic construct
DTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 363 - protein, 352 aa, Synthetic construct
DTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 364 - protein, 20 aa, Synthetic construct
SGGGGSGGGGSGGGGSGGGS

SEQ ID NO: 365 - protein, 764 aa, Synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTEVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 366 - protein, 770 aa, Synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGGGSEVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 530 aa, Synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTDTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 368 - protein, 536 aa, Synthetic construct
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGGGSDTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 369 - protein, 764 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 370 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 371 - protein, 530 aa, Synthetic construct
DTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 372 - protein, 536 aa, Synthetic construct
DTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 373 - protein, 448 aa, Synthetic construct
EVQLLESGGGLVQPGGSLRLSCAASGFTFKSYAMTWVRQAPGKGLEWVSAIVYSGGSTYYADSVKGRFTISRDNSKNTLYLQMNSLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 374 - protein, 213 aa, Synthetic construct
DTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 585 aa, Synthetic construct
EVQLQQSGPELVKPGASVKISCKTSGYTFTEYTMHWVKQSHGKSLEWIGGINPNNGGTTYNQKFKGKATLTVDKSSSTAYMELRSLTSQDSAVYYCARDYYRYGHYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 376 - protein, 213 aa, Synthetic construct
QIVLTQSPAIMSASPGEKVTMTCSVSSSVRFMHWYQQKSGTSPKRWIYDTSKLASGVPARFSGSGSGTSYSLTISSMEAEDAATYYCQQWSSNPPTFGGGTKLKIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 377 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCLSIISTLT

SEQ ID NO: 378 - protein, 133 aa, Synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCESIISTLT

SEQ ID NO: 379 - protein, 447 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 380 - protein, 404 aa, Synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 381 - protein, 404 aa, Synthetic construct
MQIPQAPWPVVWAVLQLGWRPGWFLESPDRPWNAPTFSPALLLVTEGDNATFTCSFSNASESFVLNWYRMSPSNQTDKLAAFPEDRSQPGQDCRFRVTRLPNGRDFHMSVVRARRNDSGTYLCGAISLAPKAQIKESLRAELRVTERRAEVPTAHPSPSPRPAGQFQIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - DNA, 367 bp, Synthetic construct
  source 1..367: mol_type = other DNA
gaggtgcagctgctggaaagcggcggcggactggtgcagcctggaggcagcctgcggctgtcttgtgccgcttctggcttcaccttcaaggactactgcatgacctgggtcagacaggcccctggcaagggcctcgagtgggtgtccgccatcgtgtacagcggcgggtcaacatactacgccgacagcgtgaagggcagattcacaatcagcagagataacagcaagaacaccctgtacctgcagatgaacaacctgagagctgaagataccgccgtgtactactgcgccaagtacaccagagccagctacttctacgacgccatggacgtgtggggccagggcaccaccgtgacagtgtcctcat

SEQ ID NO: 383 - DNA, 327 bp, Synthetic construct
  source 1..327: mol_type = other DNA
gagatcgtgctgacccagtctcctggcaccctgagcctgagccctggcgagagagctacactgtcatgcagagcctctcagagcatcggcaagagcttcctggcctggtaccagcaaaagcctggacaggcccctagactgctgatctacgacgccagcaccagagccgctgatatccccgccagattcagcggatctggcagcggcactgatttcaccctcaccatcagcagcctggaacccgaggacttcgccgtgtactactgccagcagtactacgactggccccccctgtcttttggcggaggcacaaaggtggaaatcaag

SEQ ID NO: 384 - protein, 122 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSS

SEQ ID NO: 385 - protein, 109 aa, Synthetic construct
EIVLTQSPGTLSLSPGERATLSCRASQSIGKSFLAWYQQKPGQAPRLLIYDASTRAADIPARFSGSGSGTDFTLTISSLEPEDFAVYYCQQYYDWPPLSFGGGTKVEIK

SEQ ID NO: 386 - protein, 10 aa, Synthetic construct
GFTFKDYCMT

SEQ ID NO: 387 - protein, 17 aa, Synthetic construct
AIVYSGGSTYYADSVKG

SEQ ID NO: 388 - protein, 13 aa, Synthetic construct
YTRASYFYDAMDV

SEQ ID NO: 389 - protein, 12 aa, Synthetic construct
RASQSIGKSFLA

SEQ ID NO: 390 - protein, 7 aa, Synthetic construct
DASTRAA

SEQ ID NO: 391 - protein, 10 aa, Synthetic construct
QQYYDWPPLS

SEQ ID NO: 392 - DNA, 363 bp, Synthetic construct
  source 1..363: mol_type = other DNA
caggttcagctggttcagtctggcagcgagctgaagaaacctggcgcctctgtgaaggtgtcctgcaaggcctctggctacagcctgtacggcacctctatgcactgggtccgacaggctccaggacagggacttgagtggatgggctacatcagcccctttaccggcagagccacatacgcccagggcttcacaggcagattcgtgttcagcctggacaccagcgtgtccacagcctacctgcagatcagctctctgaaggccgaggacaccgccgtgtactactgcgccagagactacgactaccggtactactatgccatggactactggggccagggcaccacagttacagtgtcctca

SEQ ID NO: 393 - DNA, 324 bp, Synthetic construct
  source 1..324: mol_type = other DNA
gaaattgtgctgacacagagccccgacttccagagcgtgacccctaaagaaaaagtgaccatcacctgtaccgccagcgagtccgtgcctcctcagttcctgcattggtatcagcagaagcccgatcagagccccaagctgctgatctacgccagcagagaaagagccagcggcgtcccaagcagattttctggctctggcagcggcaccgacttcaccctgacaatcaatagcctggaagccgaggacgccgccacctactactgccaccagtttcacagaagccctctgacctttggcggaggcaccaagctggaaatcaag

SEQ ID NO: 394 - protein, 121 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSS

SEQ ID NO: 395 - protein, 108 aa, Synthetic construct
EIVLTQSPDFQSVTPKEKVTITCTASESVPPQFLHWYQQKPDQSPKLLIYASRERASGVPSRFSGSGSGTDFTLTINSLEAEDAATYYCHQFHRSPLTFGGGTKLEIK

SEQ ID NO: 396 - protein, 10 aa, Synthetic construct
GYSLYGTSMH

SEQ ID NO: 397 - protein, 17 aa, Synthetic construct
YISPFTGRATYAQGFTG

SEQ ID NO: 398 - protein, 12 aa, Synthetic construct
DYDYRYYYAMDY

SEQ ID NO: 399 - protein, 12 aa, Synthetic construct
TASESVPPQFLH

SEQ ID NO: 400 - protein, 7 aa, Synthetic construct
ASRERAS

SEQ ID NO: 401 - protein, 9 aa, Synthetic construct
HQFHRSPLT

SEQ ID NO: 402 - DNA, 345 bp, Synthetic construct
  source 1..345: mol_type = other DNA
gacgtgcagctggtggaaagcggcggaggcctggtccagcccggcggctctctgagactgagctgcgccgccagcggcttcaccttcgacatcagcgccatgagctgggtgcggcaggcccctggcaagggcctggaatgggtcagcacaatcagcggatctgcctacagcacctactacgccgacagcgtgaagggcagattcaccatctcaagagataacagcaagagcaccctgtacctgcagatgaacagcctgcgggccgaggacaccgccgtgtactactgcgccagagagatcttcagcgactactggggcttgggcaccctggtgacagtgtcctca

SEQ ID NO: 403 - DNA, 330 bp, Synthetic construct
  source 1..330: mol_type = other DNA
caaagcgtgctgacacagccccccagcgcttctggcacccctggccagagagtgaccatctcatgcagcgggtcaacaagcaacatcggcagagagagcgtgtactggtaccagcagctgcctggaaccgcccctaagctgctgatctacagcaacgtgcagcggcctagcggcgcccctaacagattcagcggcagcaagagcggcaccagcgccagcctggccatcagcggcctgcagagcgaggacgaggccgactactactgcggcacatgggacgacagcctgaacggctgggtgttcggcggcggaactaagctgaccgtccta

SEQ ID NO: 404 - protein, 115 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSS

SEQ ID NO: 405 - protein, 110 aa, Synthetic construct
QSVLTQPPSASGTPGQRVTISCSGSTSNIGRESVYWYQQLPGTAPKLLIYSNVQRPSGAPNRFSGSKSGTSASLAISGLQSEDEADYYCGTWDDSLNGWVFGGGTKLTVL

SEQ ID NO: 406 - protein, 10 aa, Synthetic construct
GFTFDISAMS

SEQ ID NO: 407 - protein, 17 aa, Synthetic construct
TISGSAYSTYYADSVKG

SEQ ID NO: 408 - protein, 6 aa, Synthetic construct
EIFSDY

SEQ ID NO: 409 - protein, 13 aa, Synthetic construct
SGSTSNIGRESVY

SEQ ID NO: 410 - protein, 7 aa, Synthetic construct
SNVQRPS

SEQ ID NO: 411 - protein, 11 aa, Synthetic construct
GTWDDSLNGWV

SEQ ID NO: 412 - DNA, 1755 bp, Synthetic construct
  source 1..1755: mol_type = other DNA
gaggtgcagctgctggaaagcggcggcggcctcgtgcagcctggcggatctctgcggctgagctgtgctgccagcggcttcacatttaaatcctacgccatgcactgggttagacaagcccccggaaagggcctggaatgggtgtccgccatcgtctacagcggcggatctacatactacgccgacagcgtgaagggccggttcaccatcagcagagataatagcaagaacaccctgtacctgcagatgaacagcctgagagccgaggacaccgccgtgtactactgcgccaagtacgacagagcttcttatttctacgatgccatggacgtgtggggccagggcaccaccgtgacagtgtcctcagctagcaccaagggccctagcgtgtttccactggcccctagctctaaaagcacaagcggcggaaccgccgctctgggttgtctggtgaaggactacttccctgagcctgtgaccgtcagctggaacagcggcgccctgaccagcggcgttcacacattccccgctgtgctgcagagctctgggctgtacagcctgagcagcgtggtgaccgtgccttcttcttctctgggcacacaaacatacatctgcaacgtgaaccacaagcccagtaataccaaagtggataagaaggtggaacctaagtcttgcgacaagacccacacctgtcctccgtgccctgctcctgaactggctggagctcccagcgtgttcctgttcccccccaaacctaaagacaccctgatgatcagccggacccctgaggtgacctgcgtggtcgtcgacgtgtcccacgaagatcctgaggtgaagttcaactggtacgtggacggcgtggaagtgcataatgccaagacaaagcctagagaggaacagtacaacagcacctatagagtggtgtccgtgctgacagtgctgcaccaggactggctgaacggcaaggaatacaagtgcaaggtgtccaacaaggccctccccgcccctatcgagaagaccatcagcaaggcaaagggccaacctagagagccccaggtgtacaccctgcctccaagcagagatgagctgaccaagaaccaggttagcctgacttgtctggtgaaaggcttctacccctccgatatcgccgtggaatgggagagcaacggccagcctgagaacaactacaagaccacacctccagtgctggacagcgacggcagcttcttcctgtatagcaagctgacagtggacaagagcagatggcagcagggcaacgtgttcagctgcagcgtcatgcacgaggccctgcacaaccactacacccagaagtctctgagcctgagccctggaaaggcccctgcttctagcagcaccaagaagacccagctgcagctggaacacctgctgctggccctgcagatgatcctgaacggcatcaacaactacaagaaccccaagctgaccgagatgctgacatttaagttctacatgcctaagaaagccaccgagctgaagcacctgcaatgtctggaagaagagctgaaacctctggaagaggtgctgaatctggctcagtcaaagaacttccaccttagacctagagatctgatcagcaacatcaacgtgatcgtgctggaactgaagggcagcgagacgaccttcatgtgcgagtacgccgacgagacagccacaatcgtggagttcctgaacagatggatcaccttcgcccagagcatcatctccaccctgacc

SEQ ID NO: 413 - DNA, 648 bp, Synthetic construct
  source 1..648: mol_type = other DNA
gagatcgtgctgacccagtccccaggcacactgagcctgagccccggcgagcgggccaccctgagctgtagagctagccagagcatctccagcagcttcctggcctggtaccagcagaaacctggccaggcccctagactgctgatctacgacgcctctgatagagctacaggcatccccgaccggttcagcggcagcggatctggcaccgacttcaccctgaccatcagcagactcgagcctgaagatttcgccgtgtactactgccagcaatactatgactggcctcctctgtcttttggcggcggaacaaaggtggaaattaagcgtacggtggcggcgcccagcgtgttcatcttcccacccagcgacgagcagctgaagtccggcacagccagcgtggtgtgcctgctgaacaacttctacccccgcgaggccaaggtgcagtggaaggtggacaacgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgc

SEQ ID NO: 414 - protein, 451 aa, Synthetic construct
EVQLLESGGGLVQPGGSLRLSCAASGFTFKSYAMHWVRQAPGKGLEWVSAIVYSGGSTYYADSVKGRFTISRDNSKNTLYLQMNSLRAEDTAVYYCAKYDRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 415 - protein, 216 aa, Synthetic construct
EIVLTQSPGTLSLSPGERATLSCRASQSISSSFLAWYQQKPGQAPRLLIYDASDRATGIPDRFSGSGSGTDFTLTISRLEPEDFAVYYCQQYYDWPPLSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 416 - protein, 122 aa, Synthetic construct
EVQLLESGGGLVQPGGSLRLSCAASGFTFKSYAMHWVRQAPGKGLEWVSAIVYSGGSTYYADSVKGRFTISRDNSKNTLYLQMNSLRAEDTAVYYCAKYDRASYFYDAMDVWGQGTTVTVSS

SEQ ID NO: 417 - protein, 109 aa, Synthetic construct
EIVLTQSPGTLSLSPGERATLSCRASQSISSSFLAWYQQKPGQAPRLLIYDASDRATGIPDRFSGSGSGTDFTLTISRLEPEDFAVYYCQQYYDWPPLSFGGGTKVEIK

SEQ ID NO: 418 - protein, 10 aa, Synthetic construct
GFTFKSYAMH

SEQ ID NO: 419 - protein, 17 aa, Synthetic construct
AIVYSGGSTYYADSVKG

SEQ ID NO: 420 - protein, 13 aa, Synthetic construct
YDRASYFYDAMDV

SEQ ID NO: 421 - protein, 12 aa, Synthetic construct
RASQSISSSFLA

SEQ ID NO: 422 - protein, 7 aa, Synthetic construct
DASDRAT

SEQ ID NO: 423 - protein, 10 aa, Synthetic construct
QQYYDWPPLS

SEQ ID NO: 424 - protein, 448 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 425 - protein, 216 aa, Synthetic construct
EIVLTQSPGTLSLSPGERATLSCRASQSIGKSFLAWYQQKPGQAPRLLIYDASTRAADIPARFSGSGSGTDFTLTISSLEPEDFAVYYCQQYYDWPPLSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 426 - protein, 447 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 427 - protein, 215 aa, Synthetic construct
EIVLTQSPDFQSVTPKEKVTITCTASESVPPQFLHWYQQKPDQSPKLLIYASRERASGVPSRFSGSGSGTDFTLTINSLEAEDAATYYCHQFHRSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 428 - protein, 441 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 429 - protein, 216 aa, Synthetic construct
QSVLTQPPSASGTPGQRVTISCSGSTSNIGRESVYWYQQLPGTAPKLLIYSNVQRPSGAPNRFSGSKSGTSASLAISGLQSEDEADYYCGTWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 430 - protein, 582 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 431 - protein, 585 aa, Synthetic construct
EVQLLESGGGLVQPGGSLRLSCAASGFTFKSYAMHWVRQAPGKGLEWVSAIVYSGGSTYYADSVKGRFTISRDNSKNTLYLQMNSLRAEDTAVYYCAKYDRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 432 - protein, 587 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 433 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 434 - protein, 577 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 435 - protein, 582 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 436 - DNA, 441 bp, Synthetic construct
  source 1..441: mol_type = other DNA
ccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaa

SEQ ID NO: 437 - protein, 147 aa, Synthetic construct
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 438 - protein, 448 aa, Synthetic construct
EVQLLESGGGLVQPGGSLRLSCAASGFTFSSNYMSWVRQAPGKGLEWVSAISSSGGTIFYADSVKGRFTISRDNSKNTLYLQMNSLRAEDTAVYYCAKHKWNAVYYDGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 439 - protein, 216 aa, Synthetic construct
QSVLTQPPSASGTPGQRVTISCSGSNSNIGRNLVNWYQQLPGTAPKLLIYTIDQRPSGVPDRFSGSKSGTSASLVISGLQSEDEADYYCAAWDGSLNAWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 440 - protein, 445 aa, Synthetic construct
EVQLLESGGGLVQPGGSLRLSCTASGFTFSSYEMQWVRQAPGKGLEWVLGITSSSSHIFYADSVKGRFTVSRDNSKNTLYLQMNSLRAEDTAVYYCTKDLNSYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 441 - protein, 216 aa, Synthetic construct
QSVMTQPPSASGTPGQRVTISCSGSTSNLGNNYVSWYQHLPGTAPKLLIYGNDQRPSGVPDRFSGSKSGTSASLAISGLQSDDEADYYCSSWDASLNVWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 442 - protein, 448 aa, Synthetic construct
EVQLLESGGGLVQPGGSLRLSCAASGFTFSDYYMSWVRQAPGKGLEWVSAISSSGGTIFYADSVKGRFIISRDNSKNTLYLQMNSLRAEDTAVYYCAKHKWNDVYYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 443 - protein, 216 aa, Synthetic construct
QSVLTQPPSASGTPGQRVTISCSGSNSNIGRNLVNWYQQLPGTAPKLLIYTVDQRPSGVPDRFSGSKSGTSASLAISGLASEDEADYYCAAWDSSLNSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 444 - protein, 441 aa, Synthetic construct; identical to SEQ ID NO: 428

SEQ ID NO: 445 - protein, 216 aa, Synthetic construct
QSVLTQPPSASGTPGQRVTISCSGNTSNIGRESVYWYQQLPGTAPKLLIYSNVQRPSGVPDRFSGSKSGTSASLAISGLQSEDEADYYCGTWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 446 - protein, 440 aa, Synthetic construct
DSLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 447 - protein, 216 aa, Synthetic construct
QSVLTQPPSASGTPGQRVTISCSGSTSNIGRESVYWYQQLPGTAPKLLIYLNSQRPSGVPDRFSGSKSGTSASLAISGLQSEDVADYYCGTWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 448 - DNA, 6765 bp, Synthetic construct
  source 1..6765: mol_type = other DNA
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccggcgcgccagatctcaagcttatggacatgcgggtgccagcacaacttctcggattactattgttatggctgcgaggtgcgcgctgttatccttacgacgtgcctgactacgccccaggatggttcttagagtccccagacaggccctggaacgcccccaccttctccccagccctgctcctggtgaccgaaggggacaacgccaccttcacctgcagcttctccaacgcatcggagagcttcgtgctaaactggtacaggatgagccccagcaaccagacggacaagctggccgccttccccgaggaccgcagccagcccggccaggactgccgcttccgtgtcacacgcctgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaagccctggtggttggtgtcgtgggcggcctgctgggcagcctggtgctgctagtctgggtcctggccgtcatctgctcccgcgccgcacaagggacaatagaagccaggcgcacctgacgcgttaagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatgaaaaagcctgaactcaccgcgacgtctgttgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcaaccggtcgcggaggccatggatgcaatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagctgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 449 - protein, 447 aa, Synthetic construct
QVQLVQSGSELKKPGASVKVSCKASGYTFTSYAMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 450 - protein, 215 aa, Synthetic construct
EIVLTQSPDFQSVTPKEKVTITCRASQSIPPQFLHWYQQKPDQSPKLLIKAASQRASGVPSRFSGSGSGTDFTLTINSLEAEDAATYYCHQFHSSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 451 - protein, 579 aa, Synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 452 - protein, 214 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 453 - protein, 588 aa, Synthetic construct
EVQLLESGGGLVQPGGSLRLSCAASGFTFSSNYMSWVRQAPGKGLEWVSAISSSGGTIFYADSVKGRFTISRDNSKNTLYLQMNSLRAEDTAVYYCAKHKWNAVYYDGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 454 - protein, 585 aa, Synthetic construct
EVQLLESGGGLVQPGGSLRLSCTASGFTFSSYEMQWVRQAPGKGLEWVLGITSSSSHIFYADSVKGRFTVSRDNSKNTLYLQMNSLRAEDTAVYYCTKDLNSYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 455 - protein, 588 aa, Synthetic construct
EVQLLESGGGLVQPGGSLRLSCAASGFTFSDYYMSWVRQAPGKGLEWVSAISSSGGTIFYADSVKGRFIISRDNSKNTLYLQMNSLRAEDTAVYYCAKHKWNDVYYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 456 - protein, 575 aa, Synthetic construct; identical to SEQ ID NO: 433

SEQ ID NO: 457 - protein, 574 aa, Synthetic construct
DSLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 458 - protein, 448 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 459 - protein, 448 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 460 - protein, 448 aa, Synthetic construct
EVQLLESGGCLVQPGGSLRLSCAASGFTFSDYYMSWVRQAPGKGLEWVSAISSSGGTIFYADSVKGRFIISRDNSKNTLYLQMNSLRAEDTAVYYCAKHKWNDVYYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 461 - protein, 441 aa, Synthetic construct
DMQLVESGGGVVRPGESLRLSCTASGFTFSISAMSWVRQAPGKGLEWVSAISGTAYSTYYADSVRGRFTISRDNSKNTLYLQMNSLRAEDTAVYYCAKDNFFDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 462 - protein, 216 aa, Synthetic construct
QSVMTQPPSASGTPGQRVTISCSGVTSNIGSNSVYWYQQLPGTAPKLLIYLNSQRPSGVPDRFSGSKSGTSASLAISGLQSEDEADYYCGTWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 463 - protein, 441 aa, Synthetic construct
DVQLVESGGGVVRPGESLRLSCTASGFTFSISAMSWVRQAPGKGLEWVSAISGTAYSTYYADSVRGRFTISRDNSKNTLYLQMNSLRAEDTAVYYCAKDNFFDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 464 - protein, 441 aa, Synthetic construct
DVQLVESGGGVVRPGGSLRLSCAASGFTFSIYAMSWVRQAPGEGLEWVSHISASGGSTYYADSVKGRFAISRDNSKNTLYLQMNSLRAEDTAVYYCTTNLGSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 465 - protein, 441 aa, Synthetic construct
DVQLVESGGGVVRPGGSLRLSCAASGFTFSIYAVSWVRQAPGEGLEWVSHISASGGSTYYADSVKGRFAISRDNSKNTLYLQMNSLRAEDTAVYYCTTNLGSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 466 - protein, 216 aa, Synthetic construct
QSVLTQPPSASGTPGQRVTISCSGSYSDIGTNYVYWYQQLPGTAPKLLIFATDRRPSGVPDRFSGSKSGTSASLAISGLQSEDEADYYCGTWDDSLNVWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 467 - protein, 443 aa, Synthetic construct
DVQLVESGGGVVRPGESLRLSCAASGFTFSTDAMGWVRQAPGEGLEWVSLISGSGYSTYYADSVKGRFTISRDNSKNTLYLQMNSLTAEDTAVYYCAKNSLAFFDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 468 - protein, 216 aa, Synthetic construct
QSVLTQPPSASGTPGQRVTISCSGGSSNIGRESVNWYQQLPGTAPKLLIYSTDRRPSGVPDRFSGSKSGTSASLAISGLQSEDEADYYCGTWDNDLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 469 - protein, 449 aa, Synthetic construct
DVQLQESGPGLVKPSQSLSLTCTVTGHSITSDYAWNWIRQFPGDKLEWMGYISYSGYTTYNPSLKSRVSITRDTSKNQFFLQLNSVTTEDTATYFCARDLDYGPWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 470 - protein, 214 aa, Synthetic construct
DIQMTQSPASLSASVGETVTLTCRASENIHNYLAWYQQKQGKSPQLLVYNVKTLADGVPSRFSGSGSGTQYSLKINSLQPEDFGSYYCQHFWSSPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 471 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 472 - protein, 582 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 473 - protein, 582 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 474 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTEMLTFKFYMPKKATELKHLQCLEEELKPLEEVLNLAQSKNFHLRPRDLISNINVKVLELKGSETTFMCEYADETATIVEFLNRWITFAQSIISTLT

SEQ ID NO: 475 - protein, 582 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLDLQMILNGINNYKNPKLTEMLTFKFYMPKKATELKHLQCLEEELKPLEEVLNLAQSKNFHLRPRDLISNINVKVLELKGSETTFMCEYADETATIVEFLNRWITFAQSIISTLT

SEQ ID NO: 476 - protein, 582 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLDLQMILNGINNYKNPKLTEMLTFKFYMPKKATELKHLQCLEEELKPLEEVLNLAQSKNFHLRPRDLISNINVKVLELKGSETTFMCEYADETATIVEFLNRWITFAQSIISTLT

SEQ ID NO: 477 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTEMLTFKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 478 - protein, 582 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLDLQMILNGINNYKNPKLTEMLTFKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 479 - protein, 582 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLDLQMILNGINNYKNPKLTEMLTFKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 480 - protein, 577 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 481 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 482 - protein, 443 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 483 - protein, 346 aa, Synthetic construct; identical to SEQ ID NO: 362

SEQ ID NO: 484 - protein, 352 aa, Synthetic construct; identical to SEQ ID NO: 363

SEQ ID NO: 485 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLYLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 486 - protein, 577 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLYLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 487 - protein, 580 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPTSSSTKKTQLQLEHLLLYLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 488 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTPMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 489 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTSMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 490 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTDMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 491 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTQMLTFKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 492 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTRMLTHKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 493 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLDLQMILNGINNYKNPKLTDMLTFKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 494 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLDLQMILNGINNYKNPKLTEMLTFKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 495 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLDLQMILNGINNYKNPKLTRMLTHKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 496 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 497 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 498 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 499 - protein, 583 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 500 - protein, 586 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 501 - protein, 586 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 502 - protein, 586 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 503 - protein, 346 aa, Synthetic construct
DTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 504 - protein, 352 aa, Synthetic construct
DTVLTQSPALAVSPGERVTISCRASESVRTGVHWYQQKPGQQPKLLIYGASNLESGVPARFSGSGSGTDFTLTIDPVEADDTATYFCQQSWNDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 505 - protein, 575 aa, Synthetic construct
DVQLVESGGGVVRPGESLRLSCTASGFTFSISAMSWVRQAPGKGLEWVSAISGTAYSTYYADSVRGRFTISRDNSKNTLYLQMNSLRAEDTAVYYCAKDNFFDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 506 - protein, 577 aa, Synthetic construct
DVQLVESGGGVVRPGESLRLSCAASGFTFSTDAMGWVRQAPGEGLEWVSLISGSGYSTYYADSVKGRFTISRDNSKNTLYLQMNSLTAEDTAVYYCAKNSLAFFDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 507 - protein, 575 aa, Synthetic construct
DVQLVESGGGVVRPGGSLRLSCAASGFTFSIYAMSWVRQAPGEGLEWVSHISASGGSTYYADSVKGRFAISRDNSKNTLYLQMNSLRAEDTAVYYCTTNLGSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 508 - protein, 575 aa, Synthetic construct
DVQLVESGGGVVRPGGSLRLSCAASGFTFSIYAVSWVRQAPGEGLEWVSHISASGGSTYYADSVKGRFAISRDNSKNTLYLQMNSLRAEDTAVYYCTTNLGSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 509 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 510 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTRMLTSKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 511 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLSLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 512 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLDLQMILNGINNYKNPKLTRMLTAKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 513 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLDLQMILNGINNYKNPKLTRMLTIKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 514 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLDLQMILNGINNYKNPKLTRMLTQKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 515 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLDLQMILNGINNYKNPKLTRMLTTKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 516 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLDLQMILNGINNYKNPKLTRMLTWKFYMPKKATELKHLQCLEEELKPLEEVLNLAQSKNFHLRPRDLISNINVDVLELKGSETTFMCEYADETATIVEFLNRWITFCQSIISTLT

SEQ ID NO: 517 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLDLQMILNGINNYKNPKLTEMLTFKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 518 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLDLQMILNGINNYKNPKLTEMLTFKFYMPKKATELKHLQCLEEELKPLEEVLNLAQSKNFHLRPRDLISNINVKVLELKGSETTFMCEYADETATIVEFLNRWITFCQSIISTLT

SEQ ID NO: 519 - protein, 581 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 520 - protein, 581 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 521 - protein, 581 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 522 - protein, 586 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 523 - protein, 216 aa, Synthetic construct
EIVLTQSPGTLSLSPGERATLSCRASQSIGRSFLAWYQQKPGQAPRLLIYDASTRAADIPARFSGSGSGTDFTLTISSLEPEDFAVYYCQQYYDWPPLSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 524 - protein, 582 aa, Synthetic construct
EVQLLESGGGLVQPGGSLRLSCVGSGFNLKDYCMTWVRQAPGKGLEWVSAIVYSGGSTYYADSVKGRFTISRDNSKNTLYLQMNSLRAEDTAVYYCAKYTRGSYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 525 - protein, 216 aa, Synthetic construct
EIVLTQSPGTLSLSPGERATLSCRASQSIGRSFLAWYQQKPGQAPRLLIYDASTRAADIPDRFSGSGSGTDFTLTINRLEPEDFAVYYCQQYYDWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 526 - protein, 582 aa, Synthetic construct
EVQLLESGGGLVQPGGSLRLSCVGSGFNFKDYCMTWVRQAPGKGLEWVSAIVYSGGSTYYADSVKGRFTISRDNSKNTLYLQMNSLRAEDTAVYYCAKYTRGSYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPTSSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 527 - protein, 216 aa, Synthetic construct
EIVLTQSPGTLSLSPGERATLSCRASQSIGRSFLAWYQQKPGQAPRLLIYDASTRATDIPDRFSGSGSGTEFTLTISSLQSEDFAVYYCQQYYDWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 528 - protein, 582 aa, Synthetic construct
EVQLLESGGGLVQPGGSLRLSCVGSGFNFKDYCMTWVRQAPGKGLEWVSAIVYSGGSTYYADSVKGRFTISRDNSKNTLYLQMNSLRAEDTAVYYCAKYTRGSYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 529 - protein, 582 aa, Synthetic construct
EVQLLESGGGLVQPGGSLRLSCVGSGFNFKDYCMTWVRQAPGKGLEWVSAIVYSGGSTYYADSVKGRFTISRDNSKNTLYLQMNSLRAEDTAVYYCAKYTRGSYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 530 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 531 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 532 - protein, 585 aa, Synthetic construct
EVQLLESGGGLVQPGGSLRLSCAASGFTFKSYAMHWVRQAPGKGLEWVSAIVYSGGSTYYADSVKGRFTISRDNSKNTLYLQMNSLRAEDTAVYYCAKYDRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 533 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTEMLTFKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 534 - protein, 582 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLDLQMILNGINNYKNPKLTEMLTFKFYMPKKATELKHLQCLEEELKPLEEVLNLAQSKNFHLRPRKLISNINVIVLELKGSETTFMCEYADETATIVEFLNRWITFAQSIISTLT

SEQ ID NO: 535 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTEMLTFKFYMPKKATELKHLQCLEEELKPLEEVLNLAQSKNFHLRPRDLISNINVKVLELKGSETTFMCEYADETATIVEFLNRWITFAQSIISTLT

SEQ ID NO: 536 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTEMLTFKFYMPKKATELKHLQCLEEELKPLEEVLNLAQSKNFHLRPRDLISNINVKVLELKGSETTFMCEYADETATIVEFLNRWITFAQSIISTLT

SEQ ID NO: 537 - protein, 582 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLDLQMILNGINNYKNPKLTEMLTFKFYMPKKATELKHLQCLEEELKPLEEVLNLAQSKNFHLRPRDLISNINVKVLELKGSETTFMCEYADETATIVEFLNRWITFAQSIISTLT

SEQ ID NO: 538 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLSLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 539 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTEMLTFKFYMPKKATELKHLQCLEEELKPLEEVLNLAQSKNFHLRPRFLISNINVIVLELKGSETTFMCEYADETATIVEFLNRWITFAQSIISTLT

SEQ ID NO: 540 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTEMLTFKFYMPKKATELKHLQCLEEELKPLEEVLNLAQSKNFHLRPRDLISNINVRVLELKGSETTFMCEYADETATIVEFLNRWITFAQSIISTLT

SEQ ID NO: 541 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTEMLTFKFYMPKKATELKHLQCLEEELKPLEEVLNLAQSKNFHLRPRDLISNINVEVLELKGSETTFMCEYADETATIVEFLNRWITFAQSIISTLT

SEQ ID NO: 542 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTEMLTFKFYMPKKATELKHLQCLEEELKPLEEVLNLAQSKNFHLRPRDLISNINVSVLELKGSETTFMCEYADETATIVEFLNRWITFAQSIISTLT

SEQ ID NO: 543 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTEMLTFKFYMPKKATELKHLQCLEEELKPLEEVLNLAQSKNFHLRPRDLISNINVDVLELKGSETTFMCEYADETATIVEFLNRWITFAQSIISTLT

SEQ ID NO: 544 - protein, 585 aa, Synthetic construct
EVQLLESGGGLVQPGGSLRLSCAASGFTFKDYCMTWVRQAPGKGLEWVSAIVYSGGSTYYADSVKGRFTISRDNSKNTLYLQMNNLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEELLLDLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 545 - protein, 586 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 546 - protein, 580 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 547 - protein, 447 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 548 - protein, 447 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 549 - protein, 447 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 550 - protein, 581 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 551 - protein, 581 aa, Synthetic construct
QVQLVQSGSELKKPGASVKVSCKASGYSLYGTSMHWVRQAPGQGLEWMGYISPFTGRATYAQGFTGRFVFSLDTSVSTAYLQISSLKAEDTAVYYCARDYDYRYYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 552 - protein, 441 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 553 - protein, 441 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 554 - protein, 441 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 555 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 556 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 557 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGAPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 558 - protein, 575 aa, Synthetic construct
DVQLVESGGGLVQPGGSLRLSCAASGFTFDISAMSWVRQAPGKGLEWVSTISGSAYSTYYADSVKGRFTISRDNSKSTLYLQMNSLRAEDTAVYYCAREIFSDYWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 559 - protein, 439 aa, Synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 560 - protein, 451 aa, Synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 561 - protein, 450 aa, Synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 562 - protein, 213 aa, Synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 563 - protein, 580 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 564 - protein, 456 aa, Synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYASTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 565 - protein, 595 aa, Synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYASTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGKSGGGGSAPTSSSTKKTQLQLEHLLLDLQMILNGINNYKNPKLTRMLTKKFRMPKKATELKHLQCLEEELKPLEERLNLAQSKNFHLRPRDLISNINVIVLELKGSETTFMCEYADETATIVEFLNRWITFCQSIISTLT

SEQ ID NO: 566 - protein, 218 aa, Synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 567 - protein, 455 aa, Synthetic construct
EVQLVESGGGLVQPGRSLKLSCAASGFTFGDYSMAWVRQAPKRGLEWVANIIYDGSRTFYRDSVKGRFTISRDNAKPTLYLQMDSLRPEDTATYYCATHNYPGYAMEAWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYASTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 568 - protein, 213 aa, Synthetic construct
DTVLTQSPALPVSLGQRVNISCRATKSVSRYVHWYQQKSGQQPRLLIYTTSNLESGVPSRFSGSGSGTDFTLTIDPVEADDIANYYCQQSNEIPYTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 569 - protein, 594 aa, Synthetic construct
EVQLVESGGGLVQPGRSLKLSCAASGFTFGDYSMAWVRQAPKRGLEWVANIIYDGSRTFYRDSVKGRFTISRDNAKPTLYLQMDSLRPEDTATYYCATHNYPGYAMEAWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYASTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGKSGGGGSAPTSSSTKKTQLQLEHLLLDLQMILNGINNYKNPKLTRMLTKKFRMPKKATELKHLQCLEEELKPLEERLNLAQSKNFHLRPRDLISNINVIVLELKGSETTFMCEYADETATIVEFLNRWITFCQSIISTLT

SEQ ID NO: 570 - protein, 595 aa, Synthetic construct
EVQLVGSGGGLVQPGGSLKLSCAASGFTFSDFYMAWVRQAPTKGLEWVASISTGGGNTHYRDSVKGRFTISRDNAKSTLYLQMDSLRSEETATYYCARLLSTISTPFDYWGQGVIVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYASTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGKSGGGGSAPTSSSTKKTQLQLEHLLLDLQMILNGINNYKNPKLTRMLTKKFRMPKKATELKHLQCLEEELKPLEERLNLAQSKNFHLRPRDLISNINVIVLELKGSETTFMCEYADETATIVEFLNRWITFCQSIISTLT

SEQ ID NO: 571 - protein, 214 aa, Synthetic construct
DVVLIQSPTTLSVTPGETVSLSCRASHSVGTNLHWYQQRTNESPSLLIKYSSHSTSGIPSRFSATGSGTDFTLNISNVEFDDVASYFCQQSQKWPLTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 572 - protein, 591 aa, Synthetic construct
EVQLQQSGPELVKPGASVKISCKTSGYTFTEYTMHWVKQSHGKSLEWIGGINPNNGGTTYNQKFKGKATLTVDKSSSTAYMELRSLTSQDSAVYYCARDYYRYGHYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 573 - protein, 213 aa, Synthetic construct
QIVLTQSPAIMSASPGEKVTMTCSVSSSVRFMHWYQQKSGTSPKRWIYDTSKLASGVPARFSGSGSGTSYSLTISSMEAEDAATYYCQQWSSNPPTFGGGTKLKIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 574 - protein, 133 aa, Synthetic construct
APTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLT

SEQ ID NO: 575 - protein, 133 aa, Synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 576 - protein, 448 aa, Synthetic construct
EVQLLESGGGLVQPGGSLRLSCVGSGFNLKDYCMTWVRQAPGKGLEWVSAIVYSGGSTYYADSVKGRFTISRDNSKNTLYLQMNSLRAEDTAVYYCAKYTRGSYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 577 - protein, 448 aa, Synthetic construct
EVQLLESGGGLVQPGGSLRLSCVGSGFNFKDYCMTWVRQAPGKGLEWVSAIVYSGGSTYYADSVKGRFTISRDNSKNTLYLQMNSLRAEDTAVYYCAKYTRGSYFYDAMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 578 - protein, 446 aa, Synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 579 - protein, 218 aa, Synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 580 - DNA, 9868 bp, Synthetic construct
  source 1..9868: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaagtaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgagcccctctccctccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 581 - protein, 133 aa, Synthetic construct
APTSSSTKKTQLQLEHLLLDLQMILNGINNYKNPKLTRMLTFKFYMPKKATELKHLQCLEEELKPLEERLNLAQSKNFHLRPRDLISNINVIVLELKGSETTFMCEYADETATIVEFLNRWITFCQSIISTLT

SEQ ID NO: 582 - protein, 585 aa, Synthetic construct
EVQLLESGGGLVQPGGSLRLSCAASGFTFKSYAMTWVRQAPGKGLEWVSAIVYSGGSTYYADSVKGRFTISRDNSKNTLYLQMNSLRAEDTAVYYCAKYTRASYFYDAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAPASSSTKKTQLQLEHLLLALQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 583 - protein, 216 aa, Synthetic construct
EIVLTQSPGTLSLSPGERATLSCRASQSISSSFLAWYQQKPGQAPRLLIYDASTRATGIPDRFSGSGSGTDFTLTISRLEPEDFAVYYCQQYYDWPPLSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 584 - protein, 290 aa, Synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 585 - protein, 447 aa, Synthetic construct
EVQLVGSGGGLVQPGGSLKLSCAASGFTFSDFYMAWVRQAPTKGLEWVASISTGGGNTHYRDSVKGRFTISRDNAKSTLYLQMDSLRSEETATYYCARLLSTISTPFDYWGQGVIVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 586 - protein, 214 aa, Synthetic construct
DVVLIQSPTTLSVTPGETVSLSCRASHSVGTNLHWYQQRTNESPSLLIKYSSHSTSGIPSRFSATGSGTDFTLNISNVEFDDVASYFCQQSQKWPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 587 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLAHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 588 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLILQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 589 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLSLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 590 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLHLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 591 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLWLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 592 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLYLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 593 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLRLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 594 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLFLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 595 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRKLISNINVIVLELKGSETTFMCEYADETATIVEFLNRWITFCQSIISTLT

SEQ ID NO: 596 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLIANINVIVLELKGSETTFMCEYADETATIVEFLNRWITFCQSIISTLT

SEQ ID NO: 597 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISYINVIVLELKGSETTFMCEYADETATIVEFLNRWITFCQSIISTLT

SEQ ID NO: 598 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 599 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 600 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISEINVIVLELKGSETTFMCEYADETATIVEFLNRWITFCQSIISTLT

SEQ ID NO: 601 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISFINVIVLELKGSETTFMCEYADETATIVEFLNRWITFCQSIISTLT

SEQ ID NO: 602 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISIINVIVLELKGSETTFMCEYADETATIVEFLNRWITFCQSIISTLT

SEQ ID NO: 603 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVAVLELKGSETTFMCEYADETATIVEFLNRWITFCQSIISTLT

SEQ ID NO: 604 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLALKGSETTFMCEYADETATIVEFLNRWITFCQSIISTLT

SEQ ID NO: 605 - protein, 770 aa, Synthetic construct
EVQLVESGGGLVQPGRSLKLSCAVSGFTFSDYAMAWVRQAPKKGLEWVATISYDGSRTYYRDSVKGRFTISRDNAKITLYLQMDSLRSEDTATYYCARHGSGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTSGGGGSGGGGSGGGGSGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLKLKGSETTFMCEYADETATIVEFLNRWITFCQSIISTLT

SEQ ID NO: 606 - protein, 133 aa, Synthetic construct
APTSSSTKKTQLQLEHLLLRLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 607 - protein, 133 aa, Synthetic construct
APTSSSTKKTQLQLEHLLLNLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 608 - protein, 133 aa, Synthetic construct
APTSSSTKKTQLQLEHLLLQLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 609 - protein, 133 aa, Synthetic construct
APTSSSTKKTQLQLEHLLLELQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 610 - protein, 133 aa, Synthetic construct
APTSSSTKKTQLQLEHLLLGLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 611 - protein, 133 aa, Synthetic construct
APTSSSTKKTQLQLEHLLLILQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 612 - protein, 133 aa, Synthetic construct
APTSSSTKKTQLQLEHLLLLLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 613 - protein, 133 aa, Synthetic construct
APTSSSTKKTQLQLEHLLLKLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 614 - protein, 133 aa, Synthetic construct
APTSSSTKKTQLQLEHLLLMLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 615 - protein, 133 aa, Synthetic construct
APTSSSTKKTQLQLEHLLLFLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 616 - protein, 133 aa, Synthetic construct
APTSSSTKKTQLQLEHLLLPLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 617 - protein, 133 aa, Synthetic construct
APTSSSTKKTQLQLEHLLLTLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 618 - protein, 133 aa, Synthetic construct
APTSSSTKKTQLQLEHLLLWLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 619 - protein, 133 aa, Synthetic construct
APTSSSTKKTQLQLEHLLLYLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 620 - protein, 133 aa, Synthetic construct
APTSSSTKKTQLQLEHLLLVLQMILNGINNYKNPKLTEMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 621 - protein, 133 aa, synthetic construct; identical to SEQ ID NO: 30

(19) United States
(12) Patent Application Publication
(10) Pub. No.: US 2025/0195677 A1
(43) Pub. Date: Jun. 19, 2025

(54) USES FOR ATTENUATED IL-2 IMMUNOCONJUGATES

(71) Applicant: Cephalon LLC, West Chester, PA (US)

(72) Inventors: David S. Wilson, Jr., Redwood City, CA (US); Paul Ayton, San Francisco, CA (US)

(21) Appl. No.: 18/986,379

(22) Filed: Dec. 18, 2024

Related U.S. Application Data
(60) Provisional application No. 63/612,007, filed on Dec. 19, 2023.

(51) Int. Cl. A61K 47/68; A61K 39/00; A61P 35/00; C07K 16/28

(52) U.S. Cl. CPC A61K 47/6813; A61K 47/6849; A61P 35/00; C07K 16/2818; A61K 2039/505

(57) ABSTRACT

Disclosed herein are methods of treating a cancer in a subject using an anti-hPD-1 antibody-modified human interleukin-2 (hIL-2) immunoconjugate alone or in combination with an antagonistic anti-PD-1 antibody.

### CROSS REFERENCE TO RELATED APPLICATIONS

This application claims priority to U.S. Provisional Application No. 63/612,007, which was filed on Dec. 19, 2023, the disclosure of which is hereby incorporated by reference in its entirety.

### SEQUENCE LISTING

The instant application contains a Sequence Listing which is being submitted herewith electronically in XML format and is hereby incorporated by reference in its entirety. Said XML copy, created on Dec. 18, 2024, is named 102085.002050-BIO040-US02_SequenceListing.xml and is 697 kilobytes in size.

### TECHNICAL FIELD

Disclosed herein are methods of treating a cancer in a subject with an anti-human PD-1 (hPD-1) antibody-attenuated human interleukin-2 (hIL-2) immunoconjugate, alone or in combination with an antagonistic anti-PD-1 antibody.

### BACKGROUND

The past decade has seen dramatic benefits from immuno-oncology therapies for the treatment of cancer. The approval of immunotherapies, such as immune checkpoint inhibitors, adoptive cell therapies and cancer vaccines, having revolutionized the way cancer treatment is approached. These developments have altered the standard of care (SOC) and improved survival for several tumor types. However, while immune checkpoint inhibitors have improved clinical outcomes in a variety of tumor types, only a subset of patients show clinical responses, and a large group of responders develop acquired resistance after an initial response.

The efficacy of immune checkpoint inhibitors such as PD-1 antagonists requires patients to have a competent immune system and adequate immune cell numbers. Non-responders to PD-1 antagonists typically exhibit low tumor T-cell infiltration and poor proliferative T-cell responses to PD-1 antagonism.

Human IL-2 (hIL-2) is a Type 1 four α-helical bundle, glycosylated cytokine produced by CD4+ T cells and CD8+ T cells. Autocrine and paracrine IL-2 signaling occurs through engagement of either a high-affinity trimeric receptor complex comprising IL-2Rα (CD25), IL-2Rβ (CD122), and IL-2Rγ (CD132), or an intermediate-affinity dimeric receptor complex which comprises IL-2Rβ (CD122) and IL-2Rγ (CD132). IL-2 has dual opposing and pleiotropic roles, in that it can both stimulate T cell proliferation to generate T cell effector, T cell memory, and activated NK cells, but can also stimulate suppressive regulatory T cells for maintenance of immune homeostasis. Low-dose IL-2 primarily stimulates regulatory T cells as well as some T effector and NK cells, whereas high-dose IL-2 broadly stimulates cytotoxic T cells, T effector, and NK cells and regulatory T cells. The use of IL-2 in the treatment of autoimmune diseases and as a cancer immunotherapy has, however, been limited by off-target effects and toxicity associated with the administration of IL-2.

### SUMMARY

Disclosed herein are methods of treating a cancer in a subject, the methods comprising administering to the subject:
(A) an anti-human PD-1 (hPD-1) antibody-modified human interleukin-2 (hIL-2) immunoconjugate comprising:
   a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain complementarity determining region 1 (CDR1) comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411; and
(B) an antagonistic anti-PD-1 antibody that binds PD-1 in the presence of the immunoconjugate.

Disclosed herein are methods of treating a renal cell carcinoma, a triple negative breast cancer, a squamous cell carcinoma of the head/neck, a Merkel cell carcinoma, a hepatocellular carcinoma, or a microsatellite instability-high tumor or tumor with deficient DNA mismatch repair in a subject, the methods comprising administering to the subject:
an anti-hPD-1 antibody-modified hIL-2 immunoconjugate comprising:
   a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR 1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411.

Also disclosed herein are uses of
(A) an anti-hPD-1 antibody-modified hIL-2 immunoconjugate comprising:
   a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411; and
(B) an antagonistic anti-PD-1 antibody that binds PD-1 in the presence of the immunoconjugate for the treatment of cancer.

Also disclosed are uses of an anti-hPD-1 antibody-modified hIL-2 immunoconjugate comprising:
a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
an anti-hPD-1 antibody, or antigen-binding fragment thereof, that immunospecifically binds to hPD-1, wherein the antibody or antigen-binding fragment thereof comprises:
   (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
   (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
   (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR 1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
   (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411
for the treatment of a renal cell carcinoma, a triple negative breast cancer, a squamous cell carcinoma of the head/neck, a Merkel cell carcinoma, a hepatocellular carcinoma, or a microsatellite instability-high tumor or tumor with deficient DNA mismatch repair.

### BRIEF DESCRIPTION OF THE DRAWINGS

The summary, as well as the following detailed description, is further understood when read in conjunction with the appended drawings. For the purpose of illustrating the disclosed methods and uses, there are shown in the drawings exemplary embodiments of the methods and uses; however, the methods and uses are not limited to the specific embodiments disclosed. In the drawings:

FIG. 1A, FIG. 1B, FIG. 1C, FIG. 1D, FIG. 1E, FIG. 1F, FIG. 1G, and FIG. 1H illustrate exemplary antibody-hIL-2 immunoconjugates as described in Example 1 herein. Non-attenuated human IL-2 cytokine (grey rectangle) was fused either directly (df) or via an L6 linker (L6) to either the N-terminus or C-terminus of both heavy chains or both kappa light chains of the antibody.

FIG. 2A, FIG. 2B, FIG. 2C, FIG. 2D, FIG. 2E, FIG. 2F, FIG. 2G, and FIG. 2H illustrate exemplary antibody-hIL-2 immunoconjugates with hCD25 (1-164) extracellular domain designed to interfere with the immunoconjugate's hIL-2 binding to the human IL-2Rα. For N-terminal variants, the human CD25/IL-2Rα extracellular domain (black triangle) was fused to a non-attenuated hIL-2 cytokine (grey rectangle) via an L20 linker (light grey line). Non-attenuated hIL-2 cytokine was then either directly fused (df) or fused to the antibody with an L6 linker. For C-terminal variants, the hCD25/IL-2Rα extracellular domain moiety was either directly fused (df) or fused to the antibody using an L6 linker, followed by an L20 linker and non-attenuated hIL-2 cytokine.

FIG. 3 illustrates an exemplary 1H3-hIgG1-L6-hIL-2 immunoconjugate that contains a CD25/IL-2Rα extracellular domain moiety. The hCD25/IL-2Rα extracellular domain moiety was fused to the 1H3-hIgG1-L6-hIL-2 at the C-terminus of each heavy chain via an L6 linker followed by an L20 linker and hIL-2 cytokine moiety containing substitutions predicted to modulate binding to CD122/IL-2Rβ as described in Example 2 (attenuated hIL-2).

FIG. 4A, FIG. 4B, FIG. 4C, and FIG. 4D show the results of experiments analyzing the binding of the anti-hPD-1 antibodies 2H7-hIgG4, C51E6-5-hIgG4, and A2-hIgG4 to the human PD-1 receptor on Jurkat cells in the presence of saturating concentrations of anti-hPD-1 #1-mIgG2b-N297A and anti-hPD-1 #2-mIgG2b-N297A (10 μM) prior to exposure with anti-hPD-1 antibodies.

FIG. 5 illustrates exemplary anti-hPD-1-attenuated hIL-2 immunoconjugates either with an L6 linker (L6) (left) or direct fusion (df) (right). Anti-hPD-1 antibodies comprising either hIgG4 or hIgG1 Fc domains, with or without L235E (LE) or L235A/G237A (LAGA) modifications in the Fc domain, were fused to attenuated hIL-2 cytokines at the C-terminus of the antibody heavy chains. Various substitutions in the hIL-2 cytokine were introduced for potency attenuation.

FIG. 6A and FIG. 6B show the results of competition assays demonstrating that anti-hPD-1 #1-mIgG2b-N297A (FIG. 6A) or anti-hPD-1 #2-mIgG2b-N297A (FIG. 6B) bind to anti-hPD-1 receptor on Jurkat cells in the presence of saturating concentrations of anti-hPD-1-attenuated hIL-2 immunoconjugates (280 nM).

FIG. 7 shows the results of competition assays demonstrating that the anti-hPD-1-attenuated hIL-2 immunoconjugates 2H7-hIgG4-df-hIL-2 (D20A/R38E), C51E6-5-L6-hIgG4-hIL-2 (D20A/R38E), and A2-hIgG4-df-hIL-2 (D20A/R38E) do not inhibit the binding of human PD-L1 to the human PD-1 receptor using the PD-1/PD-L1 Blockade Bioassay.

FIG. 8 shows the results of experiments analyzing the effect of the administration of vehicle, surrogate anti-PD-1 antibodies (anti-mPD-1 RMP1-14 mIgG2b-N297A and anti-mPD-1 RMP1-30 mIgG2b-N297A), and surrogate anti-PD-1-attenuated hIL-2 immunoconjugates (anti-mPD-1 RMP1-14 mIgG2b-N297A-L6-hIL-2 (F42K/Y45R/V69R) or anti-mPD-1 RMP1-30 mIgG2b-N297A-L6-hIL-2 (F42K/Y45R/V69R)) on the growth of established subcutaneous MC38 syngeneic tumors in C57BL/6 mice, as described in Example 18. Test agents were dosed intraperitoneally at 5 mg/kg twice weekly for 4 weeks, starting on day 1. The points on the graph represent mean tumor volumes of an average of 10 mice per group.

FIG. 9A, FIG. 9B, and FIG. 9C illustrate the results of studies conducted to determine the efficacy of surrogate anti-hPD-1-attenuated hIL-2 immunoconjugate anti-mPD-1 RMP1-30 mIgG2b-N297A-hIL-2 (F42K/Y45R/V69R) in an MC38 murine colon adenocarcinoma model. FIG. 9A depicts the mean subcutaneous tumor volumes (mm³) measured every 3-4 days for 8 days after the first dose of test agents (3 doses on days 1, 4, and 8 at 5 mg/kg). Tumor growth curves represent an average of 15 animals per group. FIG. 9B summarizes results from immunophenotyping tumors by flow cytometry on day 9, showing the proportion of different CD8⁺ T cell subsets as fractions of the total CD8⁺ T cell average absolute counts. FIG. 9C illustrates immunophenotyping results on day 9 which demonstrated that there was a significant expansion of CD8⁺ T effector memory and a trend towards decreased regulatory T cells in tumors (cells/μL) following exposure to the surrogate immunoconjugate.

FIG. 10 shows the results from an experiment analyzing the acceleration of Graft vs Host Disease in NOD-Prkdc^{em26Cd52}IL-2rg^{em26Cd22}/NjuCrl (NCG) mice exposed to anti-hPD-1-attenuated hIL-2 immunoconjugates, as demonstrated by significant body weight decrease in an NCG-PBMC model.

FIG. 11A and FIG. 11B show the results of an experiment analyzing the dose-dependent expansion of cells/mL of blood CD8 Effector Memory T cells (FIG. 11A) and CD4 Effector Memory T cells (FIG. 11B) of NOD-Prkdc^{em26Cd52}IL-2rg^{em26Cd22}/NjuCrl (NCG) mice treated with 2H7-hIgG1-LAGA-df-hIL-2 (T3A/D20A/R38E/C125A) in the NCG-PBMC model.

FIG. 12 shows a decrease in cells/mL of blood regulatory T cells of NOD-Prkdc^{em26Cd52}IL-2rg^{em26Cd22}/NjuCrl (NCG) mice treated with 2H7-hIgG1-LAGA-df-hIL-2 (T3A/D20A/R38E/C125A) in the NCG-PBMC model.

FIG. 13A and FIG. 13B show that H7-632-hIgG1-LAGA-df-hIL-2 (T3A/D20A/R38E/C125A) (designated “H7-767”) (FIG. 13B) continues to bind to the human PD-1 receptor on Jurkat cells in the presence of saturating concentrations of anti-hPD-1 #1-mIgG2b-N297A and anti-hPD-1 #2-mIgG2b-N297A (10 μM) prior to exposure.

FIG. 14A and FIG. 14B are graphs showing the binding of recombinant human PD-1 captured by H7-767 immobilized to a CM5 sensor chip followed by binding of either (FIG. 14A) H7-767, KEYTRUDA®, or OPDIVO® or (FIG. 14B) PD-L1 or PD-L2, as evaluated by surface plasmon resonance (SPR).

FIG. 15 demonstrates that H7-632-hIgG1-LAGA and H7-767 do not inhibit the binding of human PD-L1 to the human PD-1 receptor using an hPD-1/hPD-L1 Blockade Bioassay.

FIG. 16A, FIG. 16B, FIG. 16C, and FIG. 16D are graphs showing the binding of anti-hPD-1-attenuated hIL-2 immunoconjugates 2H7-hIgG4-df-hIL-2 (D20A/R38E), C51E6-5-hIgG4-df-hIL-2 (D20A/R38E), and A2-hIgG4-df-hIL-2 (D20A/R38E) to the human PD-1 receptor on Jurkat cells in the presence of saturating concentrations of anti-hPD-1 #1-mIgG2b-N297A and anti-hPD-1 #2-mIgG2b-N297A (10 μM) prior to exposure with anti-hPD-1-attenuated hIL-2 immunoconjugates, as assessed by flow cytometry.

FIG. 17 is a graph showing the binding of 2H7-hIgG4-df-hIL-2 (D20A/R38E), C51E6-5-hIgG4-df-hIL-2 (D20A/R38E), A2-hIgG4-df-hIL-2 (D20A/R38E) and the irrelevant antibody control 1H3-hIgG4-df-hIL-2 (D20A/R38E) to HEK-293T cells recombinantly expressing cynomolgus PD-1, as assessed by flow cytometry.

FIG. 18A, FIG. 18B, FIG. 18C, and FIG. 18D show the antagonist activity of anti-hPD-1-attenuated hIL-2 immunoconjugates 2H7-hIgG4-df-hIL-2 (D20A/R38E), C51E6-5-hIgG4-df-hIL-2 (D20A/R38E), and A2-hIgG4-df-hIL-2 (D20A/R38E) in the presence of anti-hPD-1 #1 or anti-hPD-1 #2. FIG. 18A and FIG. 18B show results from the titration of anti-hPD-1 #1 or anti-hPD-1 #2 in the presence of fixed concentration anti-hPD-1-attenuated hIL-2 immunoconjugates. FIG. 18C and FIG. 18D show the results from the converse experiment in which anti-hPD-1-attenuated hIL-2 immunoconjugates were titrated with a fixed concentration of 100 nM of anti-hPD-1 #1 (FIG. 18C) or 100 nM of anti-hPD-1 #2 (FIG. 18D).

FIG. 19 shows the effect of the administration of various test agents including a surrogate anti-mouse PD-1/attenuated IL-2 immunoconjugate on the growth of established subcutaneous MC38 syngeneic tumors in C57BL/6 mice. Each growth curve represents the mean tumor volume of ten mice per treatment group.

FIG. 20 shows the ability of MC38 tumor cells to grow in tumor naïve mice compared to mice from the MC38 primary tumor study illustrated in FIG. 19 that were previously dosed with anti-mPD-1-hIL-2 F42K/Y45R/V69R and which had demonstrated complete long-term regression of the established primary tumor. Animals from both cohorts (10 mice per group) were subcutaneously implanted with 5×10⁵ MC38 tumor cells on the left flank contralateral to the location of the primary tumor. Mice previously exposed to the surrogate agent anti-mPD-1-hIL-2 F42K/Y45R/V69R demonstrated no tumor growth as they had developed a sustained immunity, whilst corresponding naïve mice controls demonstrated the typical growth of tumors in their flanks.

FIG. 21 shows the change in MC38 tumor volume over time in mice treated with an anti-mouse PD-1 RMP1-14, anti-mouse PD-1 RMP1-30-hIL-2 F42K/Y45R/V69R, or a combination of anti-mouse PD-1 RMP1-14 and anti-mouse PD-1 RMP1-30-hIL-2 F42K/Y45R/V69R.

### DETAILED DESCRIPTION OF ILLUSTRATIVE EMBODIMENTS

The disclosed methods and uses may be understood more readily by reference to the following detailed description taken in connection with the accompanying figures, which form a part of this disclosure. It is to be understood that the disclosed methods and uses are not limited to the specific methods and uses described and/or shown herein, and that the terminology used herein is for the purpose of describing particular embodiments by way of example only and is not intended to be limiting of the claimed methods and uses.

Unless specifically stated otherwise, any description as to a possible mechanism or mode of action or reason for improvement is meant to be illustrative only, and the disclosed methods and uses are not to be constrained by the correctness or incorrectness of any such suggested mechanism or mode of action or reason for improvement.

Throughout this text, the descriptions refer to methods of treatment and uses. Where the disclosure describes or claims a feature or embodiment associated with methods of treatment, such a feature or embodiment is equally applicable to the uses. Likewise, where the disclosure describes or claims a feature or embodiment associated with uses, such a feature or embodiment is equally applicable to the methods of treatment. Uses included within the scope of this disclosure include, but are not limited to, Swiss-style uses, first medical uses, second/further medical uses, and uses pursuant to EPC2000.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the methods and uses be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise. The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

It is to be appreciated that certain features of the disclosed methods and uses which are, for clarity, described herein in the context of separate embodiments, may also be provided in combination in a single embodiment. Conversely, various features of the disclosed methods and uses that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

The term “antibody molecule” is meant in a broad sense and includes full length immunoglobulin molecules and antigen-binding fragments thereof.

Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Antigen-binding fragment” refers to a portion of an immunoglobulin molecule that retains the antigen binding properties of the parental full length antibody (i.e., “antigen-binding fragment thereof”). Exemplary antigen binding fragments can have: heavy chain complementarity determining regions (CDR) 1, 2, and/or 3; light chain CDR 1, 2, and/or 3; a heavy chain variable region (VH); a light chain variable region (VL); and combinations thereof. Antigen binding fragments include: a Fab fragment, a monovalent fragment consisting of the VL, VH, constant light (CL), and constant heavy 1 (CH1) domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; and a domain antibody (dAb) fragment (Ward et al., Nature 341:544-546, 1989), which consists of a VH domain or a VL domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody, described for example in Int'l Pat. Pub. Nos. WO1998/44001, WO1988/01649, WO1994/13804, and WO1992/01047. These antibody fragments are obtained using techniques well known to those of skill in the art, and the fragments are screened for utility in the same manner as are full length antibodies.

The phrase “immunospecifically binds” refers to the ability of the disclosed antibody molecules to preferentially bind to its target (hPD-1 in the case of anti-hPD-1 antibody molecules) without preferentially binding other molecules in a sample containing a mixed population of molecules. Antibody molecules that immunospecifically bind hPD-1 are substantially free of other antibodies having different antigenic specificities (e.g., an anti-hPD-1 antibody is substantially free of antibodies that specifically bind antigens other than hPD-1). Antibody molecules that immunospecifically bind hPD-1, however, can have cross-reactivity to other antigens, such as orthologs of hPD-1, including Macaca fascicularis (cynomolgus monkey) PD-1. The antibody molecules disclosed herein are able to immunospecifically bind both naturally-produced hPD-1 and to PD-1 which is recombinantly produced in mammalian or prokaryotic cells.

An antibody variable region consists of four “framework” regions interrupted by three “antigen binding sites.” The antigen binding sites are defined using various terms: (i) Complementarity Determining Regions (CDRs), three in the VH (HCDR1, HCDR2, HCDR3), and three in the VL (LCDR1, LCDR2, LCDR3) are based on sequence variability (Wu and Kabat J Exp Med 132:211-50, 1970; Kabat et al., Sequences of Proteins of Immunological Interest, 5th Ed. Public Health Service, National Institutes of Health, Bethesda, Md., 1991); and (ii) “Hypervariable regions” (“HVR” or “HV”), three in the VH (H1, H2, H3) and three in the VL (L1, L2, L3) refer to the regions of the antibody variable domains which are hypervariable in structure as defined by Chothia and Lesk (Chothia and Lesk Mol Biol 196:901-17, 1987). The AbM definition of CDRs is also widely used; it is a compromise between Kabat and Chothia numbering schemes and is so-called because it was used by Oxford Molecular's AbM antibody modelling software (Rees, A. R., Searle, S. M. J., Henry, A. H. and Pedersen, J. T. (1996) In Sternberg M. J. E. (ed.), Protein Structure Prediction. Oxford University Press, Oxford, 141-172). Other terms include “IMGT-CDRs” (Lefranc et al., Dev Comparat Immunol 27:55-77, 2003) and “Specificity Determining Residue Usage” (SDRU) (Almagro Mol Recognit 17:132-43, 2004). The International ImMunoGeneTics (IMGT) database (www_imgt_org) provides a standardized numbering and definition of antigen-binding sites. The correspondence between CDRs, HVs and IMGT delineations is described in Lefranc et al., Dev Comparat Immunol 27:55-77, 2003.

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

“Human antibody,” “fully human antibody,” and like terms refers to an antibody having heavy and light chain variable regions in which both the framework and the antigen binding sites are derived from sequences of human origin. If the antibody contains a constant region, the constant region also is derived from sequences of human origin. A human antibody comprises heavy and/or light chain variable regions that are “derived from” sequences of human origin if the variable regions of the antibody are obtained from a system that uses human germline immunoglobulin or rearranged immunoglobulin genes. Such systems include human immunoglobulin gene libraries displayed on phage, and transgenic non-human animals such as mice or chicken carrying human immunoglobulin loci as described herein. “Human antibody” may contain amino acid differences when compared to the human germline or rearranged immunoglobulin sequences due to, for example, naturally occurring somatic mutations or intentional introduction of substitutions in the variable domain (framework and antigen binding sites), or constant domain. Typically, a “human antibody” is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical in amino acid sequence to an amino acid sequence encoded by a human germline or rearranged immunoglobulin gene. In some cases, a “human antibody” may contain consensus framework sequences derived from human framework sequence analyses, for example as described in Knappik et al., J Mol Biol 296:57-86, 2000, or synthetic HCDR3 incorporated into human immunoglobulin gene libraries displayed on phage, as described in, for example, Shi et al., J Mol Biol 397:385-96, 2010 and Int'l Pat. Pub. No. WO2009/085462. Antibodies in which antigen binding sites are derived from a non-human species are not included in the definition of “human antibody.”

Human antibodies, while derived from human immunoglobulin sequences, may be generated using systems such as phage display incorporating synthetic CDRs and/or synthetic frameworks, or can be subjected to in vitro mutagenesis to improve antibody properties in the variable regions or the constant regions or both, resulting in antibodies that do not naturally exist within the human antibody germline repertoire in vivo.

“Recombinant antibody” includes all antibodies that are prepared, expressed, created, or isolated by recombinant means, such as: antibodies isolated from an animal (e.g., a mouse) that is transgenic or transchromosomal for human immunoglobulin genes or a hybridoma prepared therefrom (described further below); antibodies isolated from a host cell transformed to express the antibody; antibodies isolated from a recombinant, combinatorial antibody library; and antibodies prepared, expressed, created, or isolated by any other means that involve splicing of human immunoglobulin gene sequences to other DNA sequences, or antibodies that are generated in vitro using Fab arm exchange.

“Monoclonal antibody” refers to a population of antibody molecules of a single molecular composition. A monoclonal antibody composition displays a single binding specificity and affinity for a particular epitope, or in a case of a bispecific monoclonal antibody, a dual binding specificity to two distinct epitopes. Monoclonal antibody therefore refers to an antibody population with single amino acid composition in each heavy and each light chain, except for possible well known alterations such as removal of C-terminal lysine from the antibody heavy chain. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Epitope” refers to a portion of an antigen to which an antibody specifically binds. Epitopes usually consist of chemically active (such as polar, non-polar, or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” refers to a polypeptide or a polynucleotide that differs from a reference polypeptide or a reference polynucleotide by one or more modifications for example, substitutions, insertions, or deletions. The term “mutation” as used herein is intended to mean one or more intentional substitutions which are made to a polypeptide or polynucleotide.

“Antagonistic anti-PD-1 antibody,” as used herein, refers to an antibody that binds to PD-1 and acts to at least partially inhibit the interaction between PD-1 and its ligands PD-L1 and/or PD-L2.

“Antagonistic anti-PD-1 antibody that binds PD-1 in the presence of the immunoconjugate” means that at least a portion of the antagonistic anti-PD-1 antibody detectably binds to PD-1 which had previously been exposed to the immunoconjugate, for example using a method as described in Protocol B described herein.

“Non-antagonist anti-hPD-1 antibody” refers to an anti-human PD-1 antibody that does not inhibit the ligand-induced signaling mediated by interaction between PD-1 and its ligands PD-L1 and/or PD-L2 as measured using a cell based assay or SPR as described herein.

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, and improving or remediating damage caused, directly or indirectly, by the cancer. Treatment also includes prolonging survival as compared to the expected survival of a subject not receiving treatment. Subjects to be treated include those that have the cancer as well as those prone to have the cancer or those in which the cancer is to be prevented.

As used herein, “administering to the subject” and similar terms indicate a procedure by which the disclosed immunoconjugates (monotherapies) or immunoconjugates and antagonistic anti-PD-1 antibodies (combination therapies) are injected into a subject such that target cells, tissues, or segments of the body of the subject are contacted with the disclosed immunoconjugates or immunoconjugates and antagonistic anti-PD-1 antibodies.

The phrase “therapeutically effective amount” refers to an amount of the immunoconjugate (monotherapy) or immunoconjugate and antagonistic anti-PD-1 antibodies (combination therapy), as described herein, effective to achieve a particular biological or therapeutic result such as, but not limited to, biological or therapeutic results disclosed, described, or exemplified herein. The therapeutically effective amount may vary according to factors such as the disease state, age, sex, and weight of the individual, and the ability of the immunoconjugate (monotherapy) or immunoconjugate and antagonistic anti-PD-1 antibodies (combination therapy) to cause a desired response in a subject. Exemplary indicators of a therapeutically effect amount include, for example, improved well-being of the patient, reduction of a cancer symptom, arrested or slowed progression of cancer symptoms, and/or absence of cancer symptoms.

The term “subject” as used herein is intended to mean any animal, in particular, mammals. Thus, the methods are applicable to human and nonhuman animals, although most preferably used with humans. “Subject” and “patient” are used interchangeably herein.

The disclosed modified hIL-2 proteins are also referred to as “attenuated” hIL-2 herein. As described herein the term “reduced potency” and related terms such as “reduction in potency” or “attenuation” of IL-2 activity refer to a reduction in potency of the modified hIL-2 as determined by an increased EC₅₀ value relative to the EC₅₀ value for an non-modified-hIL-2 in an IL-2-dependent assay. As described herein the reduction in potency of the modified hIL-2 will be on both the high affinity and on the intermediate affinity IL-2 receptors. The IL-2-dependent assay for determining potency may be an engineered human erythroleukemic TF1 (TF1+IL-2Rβ) or a human natural killer NK-92 cell proliferation assay as described herein. In one embodiment, the IL-2-dependent assay for determining potency is an engineered human erythroleukemic TF1 (TF1+IL-2Rβ) cell proliferation assay. In another embodiment, the IL-2-dependent assay for determining potency is a human natural killer NK-92 cell proliferation assay. Other IL-2-dependent assays for determining potency may also be a TF1+IL-2Rβ or a human natural killer NK-92 pSTAT5 assay as described herein. The non-modified-hIL-2 may be a prokaryote-expressed hIL-2 such as Proleukin® (which has the native human IL-2 amino acid sequence apart from a C125S substitution to remove an unbound cysteine, and which does not bear the normal human carbohydrate expression on residue T3), or the non-modified-hIL-2 may be an hIL-2 with the amino acid sequence of SEQ ID NO: 345 or with the amino acid sequence of SEQ ID NO: 345 with a C125S substitution, which is expressed in a mammalian cell line, such as a CHO or HEK cell line.

Immunoconjugate and fusion protein are used interchangeably herein.

### Combination Therapy

Standard of care cancer treatments may include the administration of a PD-1 antagonist checkpoint inhibitor (such as the antagonistic anti-PD-1 antibodies nivolumab, pembrolizumab, cemiplimab, dostarlimab, or retifanlimab) to antagonize PD-1 in tumor-resident T cells. Treatment with nivolumab or pembrolizumab, for example, may initially result in enhanced responses against the tumor by effector T cells, but in many cases these responses decrease over time as the chronically stimulated intratumoral T cells develop an “exhausted” phenotype characterized by reduced proliferative potential, reduced cytotoxic activity, and increased immune checkpoint molecule expression. The immunoconjugates described herein do not prevent antagonistic anti-PD-1 antibodies such as nivolumab or pembrolizumab from binding and antagonizing the PD-1 receptor. The mechanism of action of the immunoconjugates described herein and antagonistic anti-PD-1 antibodies is different and complementary. Specifically, the primary mechanism of action of PD-1 antagonists, such as antagonistic anti-PD-1 antibodies, is to prevent the immune inhibitory signal delivered through the interaction of PD-1 with PD-L1, which leads to maintaining T cells in, or restoring T cells to, their activated state. The mechanism of action of the immunoconjugates described herein, on the other hand, is via T cell-selective IL-2 signalling, which primarily induces T cell proliferation, and only subsequently promotes activation. The different modes of action and the potential for each agent to bind and be active at the same time provides a rationale for combining the therapies to enhance clinical outcomes in, for example, acquired resistance cases for PD-(L) 1 blockade therapies. This attribute enables the clinical combination of the disclosed immunoconjugates with antagonistic anti-PD-1 antibodies to enhance therapy. In tumors which initially responded to checkpoint inhibitors but have subsequently developed T cell exhaustion, for example, the administration of the disclosed immunoconjugates, which deliver the modified hIL-2 to PD-1-expressing cells, can restimulate tumor resident T cells to take on a non-exhausted phenotype and may encourage T cells to become responsive to checkpoint inhibitors again. In some embodiments, an antagonistic anti-PD-1 antibody and the immunoconjugate can be delivered concurrently or sequentially as single agents as described herein so that each agent can be administered at a concentration and for a period which optimizes the subject's anti-tumor responses.

Disclosed herein are methods of treating a cancer in a subject, the methods comprising administering to the subject:
(A) an anti-human PD-1 (hPD-1) antibody-modified human interleukin-2 (hIL-2) immunoconjugate comprising:
   a modified hIL-2 protein comprising a substitution at amino acid position 20 relative to the non-modified hIL-2 amino acid sequence of SEQ ID NO: 345 and a substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423 (referred to herein as “H7-632”);
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391 (referred to herein as “2H7”);
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401 (referred to herein as “C51E6-5”); or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411 (referred to herein as “A2”); and
(B) an antagonistic anti-PD-1 antibody that binds PD-1 in the presence of the immunoconjugate.

Suitable substitutions at amino acid position 20 of the modified hIL-2 portion of the immunoconjugates include, for example, any of a D20A, D20S, D20Q, D20M, D20I, D20V, D20N, D20G, D20T, or D20E substitution relative to the non-modified hIL-2 amino acid sequence of SEQ ID NO: 345.

Suitable substitutions at amino acid position 38 of the modified hIL-2 portion of the immunoconjugates include, for example, any of an R38E, R38N, R38G, R38H, R38I, R38L, R38M, R38F, R38P, R38S, R38T, R38W, R38Y, R38V, R38A, R38Q, R38D, or a R38K substitution relative to the non-modified hIL-2 amino acid sequence of SEQ ID NO: 345.

In some embodiments, any one of the D20A, D20S, D20Q, D20M, D20I, D20V, D20N, D20G, D20T, or D20E substitutions can be combined with an R38E substitution.

Thus, disclosed herein are methods of treating a cancer in a subject, the methods comprising administering to the subject:
(A) an anti-hPD-1 antibody-modified hIL-2 immunoconjugate comprising:
   a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423 (referred to herein as “H7-632”);
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391 (referred to herein as “2H7”);
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401 (referred to herein as “C51E6-5”); or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411 (referred to herein as “A2”); and
(B) an antagonistic anti-PD-1 antibody that binds PD-1 in the presence of the immunoconjugate.

The modified hIL-2 protein portion of the immunoconjugates can comprise the amino acid sequence of any one of SEQ ID NOs: 134-150, 307, 344, 607-611, 614, 617, or 620. The modified hIL-2 protein portion of the immunoconjugates can comprise the amino acid sequence of any one of SEQ ID NOs: 134-150, 307, 344, 608, 611, 614, or 620. The modified hIL-2 protein portion of the immunoconjugates can comprise the amino acid sequence of any one of SEQ ID NOs: 149, 307, 607-611, 614, 617, or 620. In some embodiments, the modified hIL-2 proteins comprise the amino acid sequence of SEQ ID NO: 134. In some embodiments, the modified hIL-2 proteins comprise the amino acid sequence of SEQ ID NO: 135. In some embodiments, the modified hIL-2 proteins comprise the amino acid sequence of SEQ ID NO: 136. In some embodiments, the modified hIL-2 proteins comprise the amino acid sequence of SEQ ID NO: 137. In some embodiments, the modified hIL-2 proteins comprise the amino acid sequence of SEQ ID NO: 138. In some embodiments, the modified hIL-2 proteins comprise the amino acid sequence of SEQ ID NO: 139. In some embodiments, the modified hIL-2 proteins comprise the amino acid sequence of SEQ ID NO: 140. In some embodiments, the modified hIL-2 proteins comprise the amino acid sequence of SEQ ID NO: 141. In some embodiments, the modified hIL-2 proteins comprise the amino acid sequence of SEQ ID NO: 142. In some embodiments, the modified hIL-2 proteins comprise the amino acid sequence of SEQ ID NO: 143. In some embodiments, the modified hIL-2 proteins comprise the amino acid sequence of SEQ ID NO: 144. In some embodiments, the modified hIL-2 proteins comprise the amino acid sequence of SEQ ID NO: 145. In some embodiments, the modified hIL-2 proteins comprise the amino acid sequence of SEQ ID NO: 146. In some embodiments, the modified hIL-2 proteins comprise the amino acid sequence of SEQ ID NO: 147. In some embodiments, the modified hIL-2 proteins comprise the amino acid sequence of SEQ ID NO: 148. In some embodiments, the modified hIL-2 proteins comprise the amino acid sequence of SEQ ID NO: 149. In some embodiments, the modified hIL-2 proteins comprise the amino acid sequence of SEQ ID NO: 150. In some embodiments, the modified hIL-2 proteins comprise the amino acid sequence of SEQ ID NO: 307. In some embodiments, the modified hIL-2 proteins comprise the amino acid sequence of SEQ ID NO: 344. In some embodiments, the modified hIL-2 proteins comprise the amino acid sequence of SEQ ID NO: 607. In some embodiments, the modified hIL-2 proteins comprise the amino acid sequence of SEQ ID NO: 608. In some embodiments, the modified hIL-2 proteins comprise the amino acid sequence of SEQ ID NO: 609. In some embodiments, the modified hIL-2 proteins comprise the amino acid sequence of SEQ ID NO: 610. In some embodiments, the modified hIL-2 proteins comprise the amino acid sequence of SEQ ID NO: 611. In some embodiments, the modified hIL-2 proteins comprise the amino acid sequence of SEQ ID NO: 614. In some embodiments, the modified hIL-2 proteins comprise the amino acid sequence of SEQ ID NO: 617. In some embodiments, the modified hIL-2 proteins comprise the amino acid sequence of SEQ ID NO: 620. The modified hIL-2 protein of any one of amino acid sequences SEQ ID NOs: 134-150, 307, 344, 607-611, 614, 617, or 620 can further comprise a T3A substitution and/or a C125A substitution. In some embodiments, the modified hIL-2 protein of any one of amino acid sequences SEQ ID NOs: 134-150, 307, 344, 607-611, 614, 617, or 620 further comprises a T3A substitution. In some embodiments, the modified hIL-2 protein of any one of amino acid sequences SEQ ID NOs: 134-150, 307, 344, 607-611, 614, 617, or 620 further comprises a C125A substitution. In some embodiments, the modified hIL-2 protein of any one of amino acid sequences SEQ ID NOs: 134-150, 307, 344, 607-611, 614, 617, or 620 further comprises a T3A substitution and a C125A substitution.

The modified hIL-2 protein portion of the immunoconjugates can comprise a D20A substitution relative to the non-modified hIL-2 amino acid sequence of SEQ ID NO: 345 and a R38E substitution relative to the non-modified hIL-2 amino acid sequence of SEQ ID NO: 345. The disclosed methods of treating a cancer in a subject can comprise administering to the subject:
(A) an anti-hPD-1 antibody-modified hIL-2 immunoconjugate comprising:
   a modified hIL-2 protein comprising a D20A substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and an anti-hPD-1 antibody, or antigen-binding fragment thereof, that immunospecifically binds to hPD-1, wherein the antibody or antigen-binding fragment thereof comprises:
      (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423 (referred to herein as “H7-632”);
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391 (referred to herein as “2H7”);
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401 (referred to herein as “C51E6-5”); or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411 (referred to herein as “A2”); and
(B) an antagonistic anti-PD-1 antibody that binds PD-1 in the presence of the immunoconjugate.

The modified hIL-2 protein portion of the immunoconjugates can further comprise a substitution at amino acid position 3 relative to the non-modified hIL-2 amino acid sequence of SEQ ID NO: 345. A suitable substitution includes, for example, a T3A. In some embodiments, the modified hIL-2 protein portion of the immunoconjugates comprise a T3A substitution, a D20A substitution, and a R38E substitution. In some aspects, the modified hIL-2 protein portion of the immunoconjugates comprise the amino acid sequence of SEQ ID NO: 216.

Alternatively, the modified hIL-2 protein portion of the immunoconjugates can further comprise a deletion of amino acid position 3 relative to the non-modified hIL-2 amino acid sequence of SEQ ID NO: 345. In some embodiments, the modified hIL-2 protein portion of the immunoconjugates comprise a deletion of amino acids 1-3, a D20A substitution, and a R38E substitution. In some aspects, the modified hIL-2 protein portion of the immunoconjugates comprise the amino acid sequence of SEQ ID NO: 218.

The modified hIL-2 protein portion of the immunoconjugates can further comprise a deletion or substitution at amino acid position 125 relative to the non-modified hIL-2 amino acid sequence of SEQ ID NO: 345. The substitution at amino acid position 125 can be C125A. In some embodiments, the modified hIL-2 protein portion of the immunoconjugates comprise a D20A substitution, a R38E substitution, and a C125A substitution. In some embodiments, the modified hIL-2 protein portion of the immunoconjugates comprise the amino acid sequence of SEQ ID NO: 215. In some embodiments, the modified hIL-2 protein portion of the immunoconjugates comprise a T3A substitution, a D20A substitution, a R38E substitution, and a C125A substitution. In some embodiments, the modified hIL-2 protein portion of the immunoconjugates comprise the amino acid sequence of SEQ ID NO: 217. In some embodiments, the modified hIL-2 protein portion of the immunoconjugates comprise a deletion of amino acids 1-3, a D20A substitution, a R38E substitution, and a C125A substitution. In some embodiments, the modified hIL-2 protein portion of the immunoconjugates comprise the amino acid sequence of SEQ ID NO: 219.

The modified hIL-2 protein portion of the immunoconjugates can exhibit a reduction in potency of at least about 200-fold, at least about 500-fold, at least about 1,000-fold, at least about 2,000-fold, at least about 5,000-fold, at least about 6,500-fold, or at least about 10,000-fold on the high affinity IL-2 receptor (hIL-2Rαβγ) relative to a non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the high affinity IL-2 receptor (hIL-2Rαβγ) relative to a non-modified hIL-2. A greater reduction in hIL-2 potency on the high affinity hIL-2 receptor may be possible and acceptable for the modified hIL-2 proteins described herein, but such a reduction may not be quantifiable with the methods described herein due to limits of the cell proliferation assay conditions.

In addition, the modified hIL-2 protein portion of the immunoconjugates can exhibit a reduction in potency of at least about 200-fold, at least about 500-fold, at least about 1,000-fold, at least about 2,000-fold, at least about 5,000-fold, at least about 6,500-fold, or at least about 10,000-fold on the intermediate affinity IL-2 receptor (hIL-2Rβγ) relative to a non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the intermediate affinity IL-2 receptor (hIL-2Rβγ) relative to an non-modified hIL-2.

The modified hIL-2 protein portion of the immunoconjugates can exhibit a reduction in potency of up to about 10,000-fold on the high affinity IL-2 receptor (hIL-2Rαβγ) and a reduction in potency of up to about 10,000-fold on the intermediate affinity IL-2 receptor (hIL-2Rβγ) relative to a non-modified hIL-2, for example as quantified by a comparison in EC₅₀ values in an hIL-2-dependent cell proliferation assay described herein. The modified hIL-2 protein portion of the immunoconjugates can exhibit a reduction in potency of greater than about 10,000-fold on the high affinity IL-2 receptor (hIL-2Rαβγ) and a reduction in potency of greater than about 10,000-fold on the intermediate affinity IL-2 receptor (hIL-2Rβγ) relative to a non-modified hIL-2.

The modified hIL-2 protein can be fused to the anti-hPD-1 antibody, or antigen-binding fragment thereof, portion of the immunoconjugate at the N-terminus of an antibody light chain, the C-terminus of an antibody light chain, the N-terminus of an antibody heavy chain, the C-terminus of an antibody heavy chain, the N-terminus of the antigen-binding fragment, or the C-terminus of the antigen-binding fragment. In some embodiments, the modified hIL-2 protein portion of the immunoconjugates is directly fused by a peptide bond to the anti-hPD-1 antibody, or antigen-binding fragment thereof, portion of the immunoconjugate. The modified hIL-2 protein portion of the immunoconjugates can be, for example, directly fused by a peptide bond to the C-terminal amino acid residue of the anti-hPD-1 antibody, or antigen-binding fragment thereof, portion of the immunoconjugate. In some embodiments, the modified hIL-2 protein is fused to the anti-hPD-1 antibody, or antigen-binding fragment thereof, portion of the immunoconjugate through a linker.

Fusion of the modified hIL-2 proteins to the anti-hPD-1 antibody, or antigen-binding fragment thereof, portion of the immunoconjugate can rescue the modified hIL-2 proteins' ability to activate the intermediate affinity IL-2 receptor. In some embodiments, the immunoconjugate is able to activate the intermediate affinity IL-2 receptor to a degree that is comparable to wild type hIL-2 activation of the intermediate affinity IL-2 receptor.

In some embodiments, the anti-hPD-1 antibody, or antigen-binding fragment thereof, portion of the immunoconjugates comprise a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423 (referred to herein as “H7-632”).

In some embodiments, the anti-hPD-1 antibody, or antigen-binding fragment thereof, portion of the immunoconjugates comprise a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391 (referred to herein as “2H7”).

In some embodiments, the anti-hPD-1 antibody, or antigen-binding fragment thereof, portion of the immunoconjugates comprise a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401 (referred to herein as “C51E6-5”).

In some embodiments, the anti-hPD-1 antibody, or antigen-binding fragment thereof, portion of the immunoconjugates comprise a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411 (referred to herein as “A2”).

The anti-hPD-1 antibody, or antigen-binding fragment thereof, portion of the immunoconjugates can comprise a heavy chain variable region comprising the amino acid sequence of SEQ ID NO: 416 and a light chain variable region comprising the amino acid sequence of SEQ ID NO: 417 (referred to herein as “H7-632”).

The anti-hPD-1 antibody, or antigen-binding fragment thereof, portion of the immunoconjugates can comprise a heavy chain variable region comprising the amino acid sequence of SEQ ID NO: 384 and a light chain variable region comprising the amino acid sequence of SEQ ID NO: 385 (referred to herein as “2H7”).

The anti-hPD-1 antibody, or antigen-binding fragment thereof, portion of the immunoconjugates can comprise a heavy chain variable region comprising the amino acid sequence of SEQ ID NO: 394 and a light chain variable region comprising the amino acid sequence of SEQ ID NO: 395 (referred to herein as “C51E6-5”).

The anti-hPD-1 antibody, or antigen-binding fragment thereof, portion of the immunoconjugates can comprise a heavy chain variable region comprising the amino acid sequence of SEQ ID NO: 404 and a light chain variable region comprising the amino acid sequence of SEQ ID NO: 405 (referred to herein as “A2”).

The anti-hPD-1 antibody, or antigen-binding fragment thereof, portion of the immunoconjugates can comprise an IgG1 heavy chain constant region.

The anti-hPD-1 antibody, or antigen-binding fragment thereof, portion of the immunoconjugates can have substitutions or deletions within the constant region to minimize Fc-mediated immune effector function, such as FcγRIIIA-mediated antibody-dependent cell-mediated cytotoxicity (ADCC), FcγRI- and FcγRIIa-dependent antibody-dependent cellular phagocytosis (ADCP), and C1q binding-mediated complement-dependent cytotoxicity (CDC). In some embodiments, the anti-hPD-1 antibody, or antigen-binding fragment thereof, portion of the immunoconjugates comprises a L235A substitution, wherein the amino acid numbering is according to EU numbering. In some embodiments, the anti-hPD-1 antibody, or antigen-binding fragment thereof, portion of the immunoconjugates comprises a G237A substitution, wherein the amino acid numbering is according to EU numbering. In some embodiments, the anti-hPD-1 antibody, or antigen-binding fragment thereof, portion of the immunoconjugates comprises an L235A and a G237A substitution, wherein the amino acid numbering is according to EU numbering.

The anti-hPD-1 antibody, or antigen-binding fragment thereof, portion of the immunoconjugate can comprise a heavy chain comprising the amino acid sequence of SEQ ID NO: 414 and a light chain comprising the amino acid sequence of SEQ ID NO: 415 (referred to herein as “H7-632-hIgG1-LAGA”).

The anti-hPD-1 antibody, or antigen-binding fragment thereof, portion of the immunoconjugate can comprise a heavy chain comprising the amino acid sequence of SEQ ID NO: 424 and a light chain comprising the amino acid sequence of SEQ ID NO: 425 (referred to herein as “2H7-hIgG4”).

The anti-hPD-1 antibody, or antigen-binding fragment thereof, portion of the immunoconjugate can comprise a heavy chain comprising the amino acid sequence of SEQ ID NO: 426 and a light chain comprising the amino acid sequence of SEQ ID NO: 427 (referred to herein as “C51E6-5-hIgG4”).

The anti-hPD-1 antibody, or antigen-binding fragment thereof, portion of the immunoconjugate can comprise a heavy chain comprising the amino acid sequence of SEQ ID NO: 428 and a light chain comprising the amino acid sequence of SEQ ID NO: 429 (referred to herein as “A2-hIgG4”).

The immunoconjugates can have one or more of the following properties:
Binds to PD-1 but does not inhibit PD-L1 binding to PD-1;
Binds to PD-1 in the presence of standard-of-care anti-PD-1 antibodies used in the clinic (e.g., pembrolizumab (KEYTRUDA®) or nivolumab (OPDIVO®));
Is highly selective for PD-1 and does not immunospecifically bind other related B7 family members;
Binds PD-1 on activated human T cells (EC₅₀˜0.1-0.2 nM in a flow binding assay);
The modified hIL-2 protein portion of the immunoconjugate demonstrates a measurable reduction in potency of at least about 200-fold, at least about 500-fold, at least about 1,000-fold, at least about 2,000-fold, at least about 5,000-fold, at least about 6,500-fold, or at least about 10,000-fold on the high affinity IL-2 receptor (hIL-2Rαβγ) relative to a non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the high affinity IL-2 receptor (hIL-2Rαβγ) relative to a non-modified hIL-2. A greater reduction in hIL-2 potency on the high affinity hIL-2 receptor may be possible and acceptable for the modified hIL-2 proteins described herein, but such a reduction may not be quantifiable with the methods described herein due to limits of the cell proliferation assay conditions;
The modified hIL-2 protein portion of the immunoconjugate demonstrates a measurable reduction in potency of at least about 200-fold, at least about 500-fold, at least about 1,000-fold, at least about 2,000-fold, at least about 5,000-fold, at least about 6,500-fold, or at least about 10,000-fold on the intermediate affinity IL-2 receptor (hIL-2Rβγ) relative to an non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the intermediate affinity IL-2 receptor (hIL-2Rβγ) relative to a non-modified hIL-2;
Rescues and expands PD-1-expressing human memory T cell subsets in a GvHD animal model; and
Has minimal or no impact on body weight, blood chemistry, or hematology parameters after single dose at 1 and 10 mg/kg in cynomolgus monkeys.

In some embodiments, the immunoconjugate comprises a modified hIL-2 protein comprising a T3A substitution, a R38E substitution, a D20A substitution, and a C125A substitution fused to the C-terminus of the anti-hPD-1 antibody heavy chain comprising a human IgG1 framework with a L235A substitution and a G237A substitution. In some embodiments, the immunoconjugate comprises a light chain comprising the amino acid sequence of SEQ ID NO: 415 and a heavy chain-modified hIL-2 protein fusion comprising the amino acid sequence of SEQ ID NO: 532.

Suitable antagonistic anti-PD-1 antibodies include nivolumab (OPDIVO®), pembrolizumab (KEYTRUDA®), cemiplimab (LIBTAYO®), dostarlimab (JEMPERLI), or retifanlimab (ZYNYZ™). In some embodiments, the antagonistic anti-PD-1 antibody is nivolumab. In some embodiments, the antagonistic anti-PD-1 antibody is pembrolizumab. References to nivolumab, pembrolizumab, cemiplimab, dostarlimab, or retifanlimab are intended to include any nivolumab-containing, pembrolizumab-containing, cemiplimab-containing, dostarlimab-containing, or retifanlimab-containing product approved by a regulatory authority as a biosimilar of OPDIVO®, KEYTRUDA®, LIBTAYO®, JEMPERLI, or ZYNYZ™ as the case may be.

The disclosed immunoconjugates can selectively deliver IL-2 signaling to PD-1-expressing T cells. The anti-hPD-1 antibody, or antigen-binding fragment thereof, portion of the immunoconjugate can be utilized solely to deliver the modified hIL-2 to PD-1-expressing cells and does not block PD-1 receptor function, as do classical anti-PD-1 inhibitor antibodies such as nivolumab (OPDIVO®) and pembrolizumab (KEYTRUDA®). The primary mechanism-of-action of the herein disclosed immunoconjugates is via the T cell selective activity of IL-2. The human PD-1 receptor is primarily expressed on a minor subset of T cells with potent tumor reactivity. Without being bound by theory, it is believed that targeting the modified hIL-2 protein portion of the immunoconjugate to this population of T cells can dramatically amplify anti-tumor immunity while reducing or minimizing off-target systemic IL-2-mediated toxicities mediated by cell populations that lack PD-1 expression.

The antagonistic anti-PD-1 antibody and the immunoconjugate can be administered to the subject together in a mixture, concurrently as single agents, or sequentially as single agents in any order. In some embodiments, the antagonistic anti-PD-1 antibody and the immunoconjugate are administered to the subject together in a mixture. In some embodiments, the antagonistic anti-PD-1 antibody and the immunoconjugate are administered to the subject concurrently as single agents. In some embodiments, the antagonistic anti-PD-1 antibody and the immunoconjugate are administered to the subject sequentially as single agents in any order. In some embodiments, the methods can comprise administering the immunoconjugate prior to administering the antagonistic anti-PD-1 antibody. In some embodiments, the methods can comprise administering the antagonistic anti-PD-1 antibody prior to administering the immunoconjugate. In some embodiments, the methods can comprise administering the immunoconjugate at substantially the same time as administering the antagonistic anti-PD-1 antibody. The immunoconjugate and the antagonistic anti-PD-1 antibody can be administered such that the second agent is administered while the first agent still shows a biological effect.

The immunoconjugate may be administered to subjects who are beginning to show signs of relapse/recurrence after a period of antagonistic PD-1 therapy. Thus, in some embodiments, the methods are performed on subjects who are beginning to show signs of relapse/recurrence after or during antagonistic PD-1 therapy.

Exemplary cancers that can be treated using the disclosed methods include bladder cancer, brain cancer, head and neck cancer, pancreatic cancer, lung cancer, non-small cell lung carcinoma, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, melanoma, squamous cell carcinoma, bone cancer, and kidney cancer. In some embodiments, the disclosed methods can be used to treat melanoma, Merkel cell carcinoma, non-small cell lung carcinoma, renal cell carcinoma, triple negative breast cancer, squamous cell carcinoma of the head/neck, hepatocellular carcinoma, or microsatellite instability-high tumors or tumors with deficient DNA mismatch repair.

Each and every aspect of the disclosed and claimed combination therapies, which are presented herein as methods of treatment, are equally applicable to uses of the immunoconjugates and antagonistic anti-PD-1 antibodies, including, but not limited to, Swiss-style uses, first medical uses, second/further medical uses, and uses pursuant to EPC2000. Thus, for example, disclosed herein are uses of
(A) an anti-hPD-1 antibody-modified hIL-2 immunoconjugate comprising:
   a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411; and
(B) an antagonistic anti-PD-1 antibody that binds PD-1 in the presence of the immunoconjugate
for the treatment of cancer.

### Monotherapies

Disclosed herein methods of treating a renal cell carcinoma, a triple negative breast cancer, a squamous cell carcinoma of the head/neck, a Merkel cell carcinoma, a hepatocellular carcinoma, or a microsatellite instability-high tumor or tumor with deficient DNA mismatch repair in a subject, the methods comprising administering to the subject:
an anti-hPD-1 antibody-modified hIL-2 immunoconjugate comprising:
   a modified hIL-2 protein comprising a substitution at amino acid position 20 relative to the non-modified hIL-2 amino acid sequence of SEQ ID NO: 345 and a substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof, comprises:
      (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423 (referred to herein as “H7-632”);
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391 (referred to herein as “2H7”);
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401 (referred to herein as “C51E6-5”); or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411 (referred to herein as “A2”).

Suitable substitutions at amino acid position 20 of the modified hIL-2 portion of the immunoconjugates include, for example, any of a D20A, D20S, D20Q, D20M, D20I, D20V, D20N, D20G, D20T, or D20E substitution relative to the non-modified hIL-2 amino acid sequence of SEQ ID NO: 345.

Suitable substitutions at amino acid position 38 of the modified hIL-2 portion of the immunoconjugates include, for example, any of an R38E, R38N, R38G, R38H, R38I, R38L, R38M, R38F, R38P, R38S, R38T, R38W, R38Y, R38V, R38A, R38Q, R38D, or a R38K substitution relative to the non-modified hIL-2 amino acid sequence of SEQ ID NO: 345.

In some embodiments, any one of the D20A, D20S, D20Q, D20M, D20I, D20V, D20N, D20G, D20T, or D20E substitutions can be combined with an R38E substitution.

Thus, disclosed herein are methods of treating a renal cell carcinoma, a triple negative breast cancer, a squamous cell carcinoma of the head/neck, a Merkel cell carcinoma, a hepatocellular carcinoma, or a microsatellite instability-high tumor or tumor with deficient DNA mismatch repair in a subject, the methods comprising administering to the subject:
(A) an anti-hPD-1 antibody-modified hIL-2 immunoconjugate comprising:
   a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423 (referred to herein as “H7-632”);
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR 1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391 (referred to herein as “2H7”);
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401 (referred to herein as “C51E6-5”); or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411 (referred to herein as “A2”).

The modified hIL-2 protein portion of the immunoconjugates can comprise the amino acid sequence of any one of SEQ ID NOs: 134-150, 307, 344, 607-611, 614, 617, or 620. The modified hIL-2 protein portion of the immunoconjugates can comprise the amino acid sequence of any one of SEQ ID NOs: 134-150, 307, 344, 608, 611, 614, or 620. The modified hIL-2 protein portion of the immunoconjugates can comprise the amino acid sequence of any one of SEQ ID NOs: 149, 307, 607-611, 614, 617, or 620. In some embodiments, the modified hIL-2 proteins comprise the amino acid sequence of SEQ ID NO: 134. In some embodiments, the modified hIL-2 proteins comprise the amino acid sequence of SEQ ID NO: 135. In some embodiments, the modified hIL-2 proteins comprise the amino acid sequence of SEQ ID NO: 136. In some embodiments, the modified hIL-2 proteins comprise the amino acid sequence of SEQ ID NO: 137. In some embodiments, the modified hIL-2 proteins comprise the amino acid sequence of SEQ ID NO: 138. In some embodiments, the modified hIL-2 proteins comprise the amino acid sequence of SEQ ID NO: 139. In some embodiments, the modified hIL-2 proteins comprise the amino acid sequence of SEQ ID NO: 140. In some embodiments, the modified hIL-2 proteins comprise the amino acid sequence of SEQ ID NO: 141. In some embodiments, the modified hIL-2 proteins comprise the amino acid sequence of SEQ ID NO: 142. In some embodiments, the modified hIL-2 proteins comprise the amino acid sequence of SEQ ID NO: 143. In some embodiments, the modified hIL-2 proteins comprise the amino acid sequence of SEQ ID NO: 144. In some embodiments, the modified hIL-2 proteins comprise the amino acid sequence of SEQ ID NO: 145. In some embodiments, the modified hIL-2 proteins comprise the amino acid sequence of SEQ ID NO: 146. In some embodiments, the modified hIL-2 proteins comprise the amino acid sequence of SEQ ID NO: 147. In some embodiments, the modified hIL-2 proteins comprise the amino acid sequence of SEQ ID NO: 148. In some embodiments, the modified hIL-2 proteins comprise the amino acid sequence of SEQ ID NO: 149. In some embodiments, the modified hIL-2 proteins comprise the amino acid sequence of SEQ ID NO: 150. In some embodiments, the modified hIL-2 proteins comprise the amino acid sequence of SEQ ID NO: 307. In some embodiments, the modified hIL-2 proteins comprise the amino acid sequence of SEQ ID NO: 344. In some embodiments, the modified hIL-2 proteins comprise the amino acid sequence of SEQ ID NO: 607. In some embodiments, the modified hIL-2 proteins comprise the amino acid sequence of SEQ ID NO: 608. In some embodiments, the modified hIL-2 proteins comprise the amino acid sequence of SEQ ID NO: 609. In some embodiments, the modified hIL-2 proteins comprise the amino acid sequence of SEQ ID NO: 610. In some embodiments, the modified hIL-2 proteins comprise the amino acid sequence of SEQ ID NO: 611. In some embodiments, the modified hIL-2 proteins comprise the amino acid sequence of SEQ ID NO: 614. In some embodiments, the modified hIL-2 proteins comprise the amino acid sequence of SEQ ID NO: 617. In some embodiments, the modified hIL-2 proteins comprise the amino acid sequence of SEQ ID NO: 620. The modified hIL-2 protein of any one of amino acid sequences SEQ ID NOs: 134-150, 307, 344, 607-611, 614, 617, or 620 can further comprise a T3A substitution and/or a C125A substitution. In some embodiments, the modified hIL-2 protein of any one of amino acid sequences SEQ ID NOs: 134-150, 307, 344, 607-611, 614, 617, or 620 further comprises a T3A substitution. In some embodiments, the modified hIL-2 protein of any one of amino acid sequences SEQ ID NOs: 134-150, 307, 344, 607-611, 614, 617, or 620 further comprises a C125A substitution. In some embodiments, the modified hIL-2 protein of any one of amino acid sequences SEQ ID NOs: 134-150, 307, 344, 607-611, 614, 617, or 620 further comprises a T3A substitution and a C125A substitution.

The modified hIL-2 protein portion of the immunoconjugates can comprise a D20A substitution relative to the non-modified hIL-2 amino acid sequence of SEQ ID NO: 345 and a R38E substitution relative to the non-modified hIL-2 amino acid sequence of SEQ ID NO: 345. Thus, disclosed herein are methods of treating a renal cell carcinoma, a triple negative breast cancer, a squamous cell carcinoma of the head/neck, a Merkel cell carcinoma, a hepatocellular carcinoma, or a microsatellite instability-high tumor or tumor with deficient DNA mismatch repair in a subject, the methods comprising administering to the subject:
(A) an anti-hPD-1 antibody-modified hIL-2 immunoconjugate comprising:
   a modified hIL-2 protein comprising a D20A substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423 (referred to herein as “H7-632”);
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391 (referred to herein as “2H7”);
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401 (referred to herein as “C51E6-5”); or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411 (referred to herein as “A2”).

The modified hIL-2 protein portion of the immunoconjugates can further comprise a substitution at amino acid position 3 relative to the non-modified hIL-2 amino acid sequence of SEQ ID NO: 345. A suitable substitution includes, for example, a T3A. In some embodiments, the modified hIL-2 protein portion of the immunoconjugates comprise a T3A substitution, a D20A substitution, and a R38E substitution. In some aspects, the modified hIL-2 protein portion of the immunoconjugates comprise the amino acid sequence of SEQ ID NO: 216.

Alternatively, the modified hIL-2 protein portion of the immunoconjugates can further comprise a deletion of amino acid position 3 relative to the non-modified hIL-2 amino acid sequence of SEQ ID NO: 345. In some embodiments, the modified hIL-2 protein portion of the immunoconjugates comprise a deletion of amino acids 1-3, a D20A substitution, and a R38E substitution. In some aspects, the modified hIL-2 protein portion of the immunoconjugates comprise the amino acid sequence of SEQ ID NO: 218.

The modified hIL-2 protein portion of the immunoconjugates can further comprise a deletion or substitution at amino acid position 125 relative to the non-modified hIL-2 amino acid sequence of SEQ ID NO: 345. The substitution at amino acid position 125 can be C125A. In some embodiments, the modified hIL-2 protein portion of the immunoconjugates comprise a D20A substitution, a R38E substitution, and a C125A substitution. In some embodiments, the modified hIL-2 protein portion of the immunoconjugates comprise the amino acid sequence of SEQ ID NO: 215. In some embodiments, the modified hIL-2 protein portion of the immunoconjugates comprise a T3A substitution, a D20A substitution, a R38E substitution, and a C125A substitution. In some embodiments, the modified hIL-2 protein portion of the immunoconjugates comprise the amino acid sequence of SEQ ID NO: 217. In some embodiments, the modified hIL-2 protein portion of the immunoconjugates comprise a deletion of amino acids 1-3, a D20A substitution, a R38E substitution, and a C125A substitution. In some embodiments, the modified hIL-2 protein portion of the immunoconjugates comprise the amino acid sequence of SEQ ID NO: 219.

The modified hIL-2 protein portion of the immunoconjugates can exhibit a reduction in potency of at least about 200-fold, at least about 500-fold, at least about 1,000-fold, at least about 2,000-fold, at least about 5,000-fold, at least about 6,500-fold, or at least about 10,000-fold on the high affinity IL-2 receptor (hIL-2Rαβγ) relative to a non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the high affinity IL-2 receptor (hIL-2Rαβγ) relative to a non-modified hIL-2. A greater reduction in hIL-2 potency on the high affinity hIL-2 receptor may be possible and acceptable for the modified hIL-2 proteins described herein, but such a reduction may not be quantifiable with the methods described herein due to limits of the cell proliferation assay conditions.

In addition, the modified hIL-2 protein portion of the immunoconjugates can exhibit a reduction in potency of at least about 200-fold, at least about 500-fold, at least about 1,000-fold, at least about 2,000-fold, at least about 5,000-fold, at least about 6,500-fold, or at least about 10,000-fold on the intermediate affinity IL-2 receptor (hIL-2Rβγ) relative to an non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the intermediate affinity IL-2 receptor (hIL-2Rβγ) relative to an non-modified hIL-2.

The modified hIL-2 protein portion of the immunoconjugates can exhibit a reduction in potency of up to about 10,000-fold on the high affinity IL-2 receptor (hIL-2Rαβγ) and a reduction in potency of up to about 10,000-fold on the intermediate affinity IL-2 receptor (hIL-2Rβγ) relative to a non-modified hIL-2, for example as quantified by a comparison in EC₅₀ values in an hIL-2-dependent cell proliferation assay described herein. The modified hIL-2 protein portion of the immunoconjugates can exhibit a reduction in potency of greater than about 10,000-fold on the high affinity IL-2 receptor (hIL-2Rαβγ) and a reduction in potency of greater than about 10,000-fold on the intermediate affinity IL-2 receptor (hIL-2Rβγ) relative to a non-modified hIL-2.

The modified hIL-2 protein can be fused to the anti-hPD-1 antibody, or antigen-binding fragment thereof, portion of the immunoconjugate at the N-terminus of an antibody light chain, the C-terminus of an antibody light chain, the N-terminus of an antibody heavy chain, the C-terminus of an antibody heavy chain, the N-terminus of the antigen-binding fragment, or the C-terminus of the antigen-binding fragment. In some embodiments, the modified hIL-2 protein portion of the immunoconjugates is directly fused by a peptide bond to the anti-hPD-1 antibody, or antigen-binding fragment thereof, portion of the immunoconjugate. The modified hIL-2 protein portion of the immunoconjugates can be, for example, directly fused by a peptide bond to the C-terminal amino acid residue of the anti-hPD-1 antibody, or antigen-binding fragment thereof, portion of the immunoconjugate. In some embodiments, the modified hIL-2 protein is fused to the anti-hPD-1 antibody, or antigen-binding fragment thereof, portion of the immunoconjugate through a linker.

Fusion of the modified hIL-2 proteins to the anti-hPD-1 antibody, or antigen-binding fragment thereof, portion of the immunoconjugate can rescue the modified hIL-2 proteins' ability to activate the intermediate affinity IL-2 receptor. In some embodiments, the immunoconjugate is able to activate the intermediate affinity IL-2 receptor to a degree that is comparable to wild type hIL-2 activation of the intermediate affinity IL-2 receptor.

In some embodiments, the anti-hPD-1 antibody, or antigen-binding fragment thereof, portion of the immunoconjugates comprise a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423 (referred to herein as “H7-632”).

In some embodiments, the anti-hPD-1 antibody, or antigen-binding fragment thereof, portion of the immunoconjugates comprise a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391 (referred to herein as “2H7”).

In some embodiments, the anti-hPD-1 antibody, or antigen-binding fragment thereof, portion of the immunoconjugates comprise a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401 (referred to herein as “C51E6-5”).

In some embodiments, the anti-hPD-1 antibody, or antigen-binding fragment thereof, portion of the immunoconjugates comprise a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411 (referred to herein as “A2”).

The anti-hPD-1 antibody, or antigen-binding fragment thereof, portion of the immunoconjugates can comprise a heavy chain variable region comprising the amino acid sequence of SEQ ID NO: 416 and a light chain variable region comprising the amino acid sequence of SEQ ID NO: 417 (referred to herein as “H7-632”).

The anti-hPD-1 antibody, or antigen-binding fragment thereof, portion of the immunoconjugates can comprise a heavy chain variable region comprising the amino acid sequence of SEQ ID NO: 384 and a light chain variable region comprising the amino acid sequence of SEQ ID NO: 385 (referred to herein as “2H7”).

The anti-hPD-1 antibody, or antigen-binding fragment thereof, portion of the immunoconjugates can comprise a heavy chain variable region comprising the amino acid sequence of SEQ ID NO: 394 and a light chain variable region comprising the amino acid sequence of SEQ ID NO: 395 (referred to herein as “C51E6-5”).

The anti-hPD-1 antibody, or antigen-binding fragment thereof, portion of the immunoconjugates can comprise a heavy chain variable region comprising the amino acid sequence of SEQ ID NO: 404 and a light chain variable region comprising the amino acid sequence of SEQ ID NO: 405 (referred to herein as “A2”).

The anti-hPD-1 antibody, or antigen-binding fragment thereof, portion of the immunoconjugates can comprise an IgG1 heavy chain constant region.

The anti-hPD-1 antibody, or antigen-binding fragment thereof, portion of the immunoconjugates can have substitutions or deletions within the constant region to minimize Fc-mediated immune effector function, such as FcγRIIIA-mediated antibody-dependent cell-mediated cytotoxicity (ADCC), FcγRI- and FcγRIIa-dependent antibody-dependent cellular phagocytosis (ADCP), and C1q binding-mediated complement-dependent cytotoxicity (CDC). In some embodiments, the anti-hPD-1 antibody, or antigen-binding fragment thereof, portion of the immunoconjugates comprises a L235A substitution, wherein the amino acid numbering is according to EU numbering. In some embodiments, the anti-hPD-1 antibody, or antigen-binding fragment thereof, portion of the immunoconjugates comprises a G237A substitution, wherein the amino acid numbering is according to EU numbering. In some embodiments, the anti-hPD-1 antibody, or antigen-binding fragment thereof, portion of the immunoconjugates comprises an L235A and a G237A substitution, wherein the amino acid numbering is according to EU numbering.

The anti-hPD-1 antibody, or antigen-binding fragment thereof, portion of the immunoconjugate can comprise a heavy chain comprising the amino acid sequence of SEQ ID NO: 414 and a light chain comprising the amino acid sequence of SEQ ID NO: 415 (referred to herein as “H7-632-hIgG1-LAGA”).

The anti-hPD-1 antibody, or antigen-binding fragment thereof, portion of the immunoconjugate can comprise a heavy chain comprising the amino acid sequence of SEQ ID NO: 424 and a light chain comprising the amino acid sequence of SEQ ID NO: 425 (referred to herein as “2H7-hIgG4”).

The anti-hPD-1 antibody, or antigen-binding fragment thereof, portion of the immunoconjugate can comprise a heavy chain comprising the amino acid sequence of SEQ ID NO: 426 and a light chain comprising the amino acid sequence of SEQ ID NO: 427 (referred to herein as “C51E6-5-hIgG4”).

The anti-hPD-1 antibody, or antigen-binding fragment thereof, portion of the immunoconjugate can comprise a heavy chain comprising the amino acid sequence of SEQ ID NO: 428 and a light chain comprising the amino acid sequence of SEQ ID NO: 429 (referred to herein as “A2-hIgG4”).

The immunoconjugates can have one or more of the following properties:
Binds to PD-1 but does not inhibit PD-L1 binding to PD-1;
Binds to PD-1 in the presence of standard-of-care anti-PD-1 antibodies used in the clinic (e.g., pembrolizumab (KEYTRUDA®) or nivolumab (OPDIVO®));
Is highly selective for PD-1 and does not immunospecifically bind other related B7 family members;
Binds PD-1 on activated human T cells (EC₅₀˜0.1-0.2 nM in a flow binding assay);
The modified hIL-2 protein portion of the immunoconjugate demonstrates a measurable reduction in potency of at least about 200-fold, at least about 500-fold, at least about 1,000-fold, at least about 2,000-fold, at least about 5,000-fold, at least about 6,500-fold, or at least about 10,000-fold on the high affinity IL-2 receptor (hIL-2Rαβγ) relative to a non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the high affinity IL-2 receptor (hIL-2Rαβγ) relative to a non-modified hIL-2. A greater reduction in hIL-2 potency on the high affinity hIL-2 receptor may be possible and acceptable for the modified hIL-2 proteins described herein, but such a reduction may not be quantifiable with the methods described herein due to limits of the cell proliferation assay conditions;
The modified hIL-2 protein portion of the immunoconjugate demonstrates a measurable reduction in potency of at least about 200-fold, at least about 500-fold, at least about 1,000-fold, at least about 2,000-fold, at least about 5,000-fold, at least about 6,500-fold, or at least about 10,000-fold on the intermediate affinity IL-2 receptor (hIL-2Rβγ) relative to an non-modified hIL-2, for example as quantified by a comparison in EC₅₀ values in an hIL-2-dependent cell proliferation assay described herein. In some embodiments, the modified hIL-2 protein portion of the immunoconjugates can exhibit a reduction in potency of greater than about 10,000-fold on the intermediate affinity IL-2 receptor (hIL-2Rβγ) relative to a non-modified hIL-2;
Rescues and expands PD-1-expressing human memory T cell subsets in a GvHD animal model; and
Has minimal or no impact on body weight, blood chemistry, or hematology parameters after single dose at 1 and 10 mg/kg in cynomolgus monkeys.

In some embodiments, the immunoconjugate comprises a modified hIL-2 protein comprising a T3A substitution, a R38E substitution, a D20A substitution, and a C125A substitution fused to the C-terminus of the anti-hPD-1 antibody heavy chain comprising a human IgG1 framework with a L235A substitution and a G237A substitution. In some embodiments, the immunoconjugate comprises a light chain comprising the amino acid sequence of SEQ ID NO: 415 and a heavy chain-modified hIL-2 protein fusion comprising the amino acid sequence of SEQ ID NO: 532.

The disclosed immunoconjugates can selectively deliver IL-2 signaling to PD-1-expressing T cells. The anti-hPD-1 antibody, or antigen-binding fragment thereof, portion of the immunoconjugate can be utilized solely to deliver the modified hIL-2 to PD-1-expressing cells and does not block PD-1 receptor function, as do classical anti-PD-1 inhibitor antibodies such as nivolumab (OPDIVO®) and pembrolizumab (KEYTRUDA®). The primary mechanism-of-action of the herein disclosed immunoconjugates is via the T cell selective activity of IL-2. The human PD-1 receptor is primarily expressed on a minor subset of T cells with potent tumor reactivity. Without being bound by theory, it is believed that targeting the modified hIL-2 protein portion of the immunoconjugate to this population of T cells can dramatically amplify anti-tumor immunity while reducing or minimizing off-target systemic IL-2-mediated toxicities mediated by cell populations that lack PD-1 expression.

Each and every aspect of the disclosed and claimed monotherapies, which are presented herein as methods of treatment, are equally applicable to uses of the immunoconjugates, including, but not limited to, Swiss-style uses, first medical uses, second/further medical uses, and uses pursuant to EPC2000. Thus, for example, disclosed herein are uses of an anti-hPD-1 antibody-modified hIL-2 immunoconjugate comprising:
a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
an anti-hPD-1 antibody, or antigen-binding fragment thereof, that immunospecifically binds to hPD-1, wherein the antibody or antigen-binding fragment thereof comprises:
   (i) a heavy chain CDR1 comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
   (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
   (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
   (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411,
for the treatment of a renal cell carcinoma, a triple negative breast cancer, a squamous cell carcinoma of the head/neck, a Merkel cell carcinoma, a hepatocellular carcinoma, or a microsatellite instability-high tumor or tumor with deficient DNA mismatch repair.

### EXAMPLES

The following examples are provided to further describe some of the embodiments disclosed herein. The examples are intended to illustrate, not to limit, the disclosed embodiments.

### General Methods

Protocol A. Flow Cytometry Screen for Binding of Anti-hPD-1 Antibodies or Anti-hPD-1 Antibody-Attenuated hIL-2 Fusions to Human PD-1

To test for binding to hPD-1, antibodies and antibody-attenuated hIL-2 fusion proteins were characterized in full titration curves. A Jurkat cell line was transfected with a mammalian vector which encoded amino acids 1-185 of human PD-1 (SEQ ID NO: 346) to stably express the extracellular domain and a portion of the transmembrane domain of human PD-1, and this transfected cell line was used to determine binding of anti-hPD-1 antibodies. Jurkat+hPD-1 cells were washed and added to 96-well plates at 100,000 cells per well in FACS buffer (PBS, 0.2% Heat-inactivated Fetal Bovine Serum). Cells were blocked with 1:50 dilution of human FcR Block (Miltenyi) for 10 minutes at 4° C. and washed with FACS buffer.

Antibodies or antibody-attenuated hIL-2 immunoconjugates (fusion proteins) were serially diluted six-fold in FACS buffer for an 8-point curve and added to human PD-1 expressing Jurkat cells for 1 hour on ice in 100 μL volume. Cells were washed and re-suspended in FACS buffer containing 1:40 dilution of Allophycocyanin conjugated anti-human IgG Fc monoclonal antibody. Cells were washed once more, re-suspended in FACS buffer containing 1:1000 dilution of Sytox Green (Thermo Fisher) and flow cytometric analysis was conducted on the BD FACS Canto II, BD Celesta or BD Fortessa (BD Biosciences) flow cytometers. The geometric mean fluorescent intensity (gMFI) was calculated using FlowJo software version 10. Half maximal effective concentration (EC₅₀) values were calculated from the gMFI of the Allophycocyanin signal across the titrated concentrations using GraphPad Prism 7 software.

Protocol B. Flow Cytometry Competition Screen for Binding of Anti-hPD-1 Antibodies or Anti-hPD-1 Antibody-Attenuated hIL-2 Fusions to Human PD-1

Antibodies and antibody-attenuated hIL-2 fusion proteins were tested for the ability to bind human PD-1 in the presence of a saturating concentration of anti-hPD-1 #1-mIgG2b-N297A (sequence comprising the heavy and light chain variable region sequences of nivolumab, clone 5C4, as described in U.S. Patent Pub. No. US 2009/0217401A1, formatted onto a murine IgG2b-N297A background) (SEQ ID NOs: 348 and 349) or anti-hPD-1 #2-mIgG2b-N297A (sequence comprising the heavy and light chain variable region sequences of pembrolizumab (clone 109A-H/K09A-L-11) as described in Int'l Pub. No. WO2008/156712A1, formatted onto a murine IgG2b-N297A background) (SEQ ID NOs: 350 and 351).

Antibodies or antibody-attenuated hIL-2 fusion proteins were serially diluted six-fold for an 8-point titration curve with and without saturating amounts of 10 UM anti-hPD-1 #1-mIgG2b-N297A or anti-hPD-1 #2-mIgG2b-N297A. Briefly, Jurkat cells stably expressing hPD-1 (as described in Protocol A above) were washed and re-suspended in FACS buffer containing 1:50 dilution of human FcR Blocking reagent. Cells were incubated at 4° C. for 10 minutes and washed. Cells were then re-suspended in 100 μL volume with anti-hPD-1 #1-mIgG2b-N297A or anti-hPD-1 #2-mIgG2b-N287A diluted in FACS buffer to 10 UM and incubated at 4° C. for one hour. Cells were washed and incubated with test antibodies or antibody-attenuated hIL-2 fusion proteins serially diluted six-fold for an 8-point curve in 100 μL volume for one hour at 4° C. To detect bound test anti-hPD-1 antibodies or anti-hPD-1-attenuated hIL-2 fusion proteins, cells were washed again and incubated with 1:40 dilution of Allophycocyanin-conjugated anti-human IgG Fc monoclonal antibody for 45 minutes on ice. Cells were washed and re-suspended in FACS buffer containing 1:1000 dilution of Sytox Green (Thermo Fisher). To generate a comparison, Jurkat cells stably expressing human PD-1 were incubated with only the titrated test antibodies or antibody-attenuated hIL-2 fusion proteins (without anti-hPD-1 #1-mIgG2b-N297A or anti-hPD-1 #2-mIgG2b-N297A) and subsequently with 1:40 dilution of Allophycocyanin-conjugated anti-human IgG Fc secondary. As a control, the variable regions of anti-hPD-1 #1 and anti-hPD-1 #2 were cloned into hIgG4 frameworks and were assessed with and without the addition of anti-hPD-1 #1-mIgG2b-N297A or anti-hPD-1 #2-mIgG2b-N297A. Flow cytometry was carried out on the BD Canto II, BD Celesta, or BD Fortessa (BD Biosciences) flow cytometers and gMFI was calculated using FlowJo software version 10. EC₅₀ values were calculated from the gMFI of the Allophycocyanin signal across the titrated concentrations using GraphPad Prism 7 software.

Protocol C. Cell-Based Screen for Characterization of Non-Antagonist Anti-hPD-1 Antibodies or Anti-hPD-1 Antibody-Attenuated hIL-2 Fusions

Human PD-1 antibodies and anti-hPD-1-attenuated hIL-2 fusion proteins were characterized for the ability to block hPD-1 from binding to ligand hPD-L1 (SEQ ID NO: 584). Anti-hPD-1 antibodies and anti-hPD-1-attenuated hIL-2 fusion proteins were either characterized as an antagonist or non-antagonist using an in vitro cell-based human PD-1/PD-L1 blockade bioassay (Promega, Cat #J1255). This co-culture assay utilized two cell lines: FCγR11b artificial Antigen Presenting Cells/Chinese Ovary Hamster K1 (aAPC/CHO-K1) and Jurkat Effector cells. aAPC/CHO K1 cells stably express both human PD-L1 ligand and a cell surface protein to activate cognate T cell receptors (TCRs) while Jurkat Effector cells express hPD-1 and a luciferase reporter under the control of Nuclear Factor of Activated T cells response element (NFAT-RE). When these cells are co-cultured in the presence of a non-antagonistic antibody, hPD-1/hPD-L1 interaction inhibits TCR signaling and no luminescence is detected. In the presence of an antibody that antagonizes hPD-1 interaction with hPD-L1 (SEQ ID NO: 584), the inhibitory signal is disrupted and luminescence is detected.

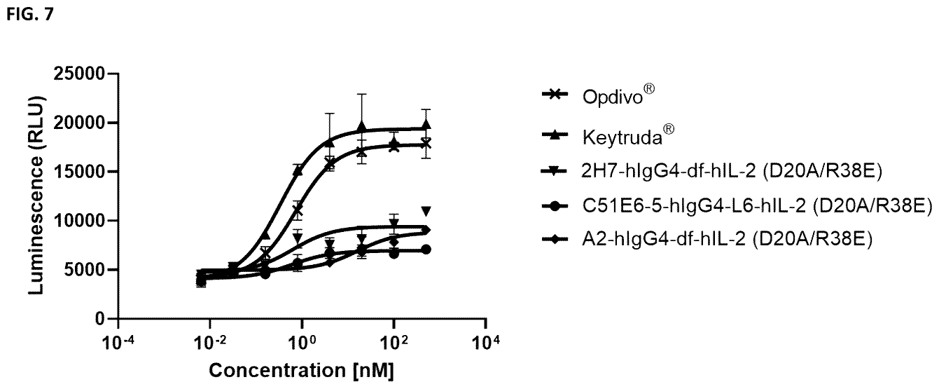

The thaw-and-use assay was performed according to manufacturer's instructions. In short, aAPC/CHO-K1 cells were first thawed and plated at 30,000 cells per well in flat-bottom 96-well plates for 18 hours at 37° C. in a 5% CO₂ incubator. After cells had adhered, the media was removed and 200 nM or 1000 nM test antibodies or antibody-attenuated hIL-2 fusion proteins were diluted in 40 μL assay buffer (RPMI 1640 medium+1% FBS) and added to the aAPC/CHO-K1 cells. A human IgG4 isotype control monoclonal antibody which targeted Keyhole Limpet Hemocyanin (KLH) clone C3 (SEQ ID NOs: 585 and 586) was used as a negative control. Jurkat effector cells expressing hPD-1 were added at 24,000 cells per well in 40 μL volume. The final concentration of fixed antibodies tested was 100 nM or 500 nM. In some examples, a range of concentrations of anti-hPD-11 or anti-hPD-1-attenuated hIL-2 fusion proteins were tested in this co-culture assay, with the top concentration in a five-fold titration series of 500 nM (FIG. 7).

The co-culture assay was incubated at 37° C. in a 5% CO₂ incubator for an additional 18-20 hours. To read the luminescence signal, plates were allowed to come to room temperature, and 80 μL of the Bio-Glo™ reagent was added to each well. The plates were incubated for 15 minutes in the dark at room temperature and luminescence was read on a Victor X luminometer (Perkin Elmer). Relative luminescence units (RLU) were averaged for each triplicate and graphed using GraphPad Prism 7 software.

Protocol D. In Vitro Phosphorylated STAT5 Assay to Test Attenuation of hIL-2 Variants

The level of attenuation of hIL-2 receptor activation activity of antibody-attenuated hIL-2 fusion proteins was characterized using a phosphorylated STAT5 assay. Variants were tested in both hIL-2 responsive human natural killer NK-92 cells and engineered human erythroleukemic TF1 cells. The NK-92 cell line naturally expresses the high-affinity hIL-2 receptor (IL-2Rαβγ) at physiologic levels, while the TF1 cell line that naturally expresses the IL-2Rγ (SEQ ID NO: 352) was engineered to also stably express human CD122 (IL-2Rβ) (SEQ ID NO: 353) for expression of the intermediate affinity hIL-2 receptor complex (IL-2Rβγ). This TF1+IL-2Rβ stable cell line does not express the IL-2Rα (SEQ ID NO: 354). Both NK-92 and TF1+IL-2Rβ cell lines were used to assess the level of attenuation of IL-2 potency in these cell-based potency assays as fixed concentration screens and full titration curves.

To perform the fixed concentration screen, 100,000 NK-92 cells or TF1+IL-2Rβ cells were plated into 96 wells in 50 μL of fresh growth medium lacking human IL-2 cytokine and incubated overnight at 37° C. in a CO₂ incubator. After 15-16 hours, human IL-2 starved cells were treated with 25.7 nM recombinant hIL-2 (denoted as rhIL-2) (SEQ ID NO: 345) or test antibody-attenuated hIL-2 fusion proteins for the NK-92 cell assay, or with 33.3 nM hIL-2 or test hIL-2 variants for the TF1+IL-2Rβ cell assay. Cells were incubated at 37° C., 5% CO₂ for 10 minutes. Cells were fixed with Cytofix Buffer (BD Biosciences) for 10 minutes at 37° C. and then permeabilized after treatment with Perm Buffer III (BD Biosciences) for 30 minutes on ice. hIL-2-dependent Stat5 phosphorylation was detected after staining fixed and permeabilized cells with Alexa Fluor-647 conjugated anti-Stat5 antibody (BD Biosciences) at 0.5 μL per sample for 45 minutes at room temperature in the dark. Cells were washed and reagents were diluted in BD Pharmingen Buffer (BD Biosciences). Stained cells were acquired on a FACS-Celesta cytometer (BD Biosciences) and analyzed using FlowJo software version 10.7.2. The assays were performed in cohorts but normalized using the rhIL-2 for each plate. The degree of attenuation of selected antibody-attenuated hIL-2 fusion proteins were evaluated in an 8-point, 6-fold serially titrated curve ranging from 1200 nM to 7 pM on both NK-92 and TF1+IL-2Rβ cell lines. The procedure for the pStat5 curves was performed in the same manner as the method described above. EC₅₀ values were calculated from the geometric mean fluorescent intensity (gMFI) across the titrated concentrations using GraphPad Prism 7 software. The fold change in activity from rhIL-2 was calculated by dividing the EC₅₀ values for the variants by the EC₅₀ of hIL-2.

Protocol E. In Vitro Cell-Based Proliferation Assay to Test Attenuation of Antibody-Attenuated hIL-2 Fusion Proteins

The antibody-attenuated hIL-2 fusion proteins were also tested for attenuated hIL-2 activity in hIL-2 dependent cell proliferation assays. 10,000 NK-92 cells (expressing the high affinity receptor hIL-2Rαβγ) or TF1+IL-2Rβ cells (expressing the intermediate affinity receptor hIL-2Rβγ) suspended in 50 μL of fresh growth medium without hIL-2 cytokine were plated per well in 96-well U-bottom cell culture plate. Eight point, 6-fold serial titrations of antibody-attenuated hIL-2 fusion proteins with a highest concentration of 996 nM were diluted in fresh media and overlaid on cells in wells. Cells were incubated at 37° C. in a 5% CO₂ incubator for 3 days for TF1+IL-2Rβ cells or 4 days for NK-92 cells. To measure proliferation, Cell-Titer-Glo (Promega) was added to wells, incubated for 10 minutes at room temperature and luminescence was read for 0.1 second per well using a VictorX Multilabel Plate Reader (Perkin Elmer). EC₅₀ values were calculated from the relative luminescence units (RLU) across the titrated concentrations using GraphPad Prism 7 software. The fold change in activity from rhIL-2 was calculated by dividing the EC₅₀ values for the variants by the EC₅₀ of hIL-2. The assays were performed in cohorts but normalized using the rhIL-2 EC₅₀ value for each plate.

### Example 1: Optimization of Antibody-Attenuated hIL-2 Fusion Protein Variants and Determination of their hIL-2 Activity on the Intermediate and High-Affinity hIL-2 Receptor Complexes

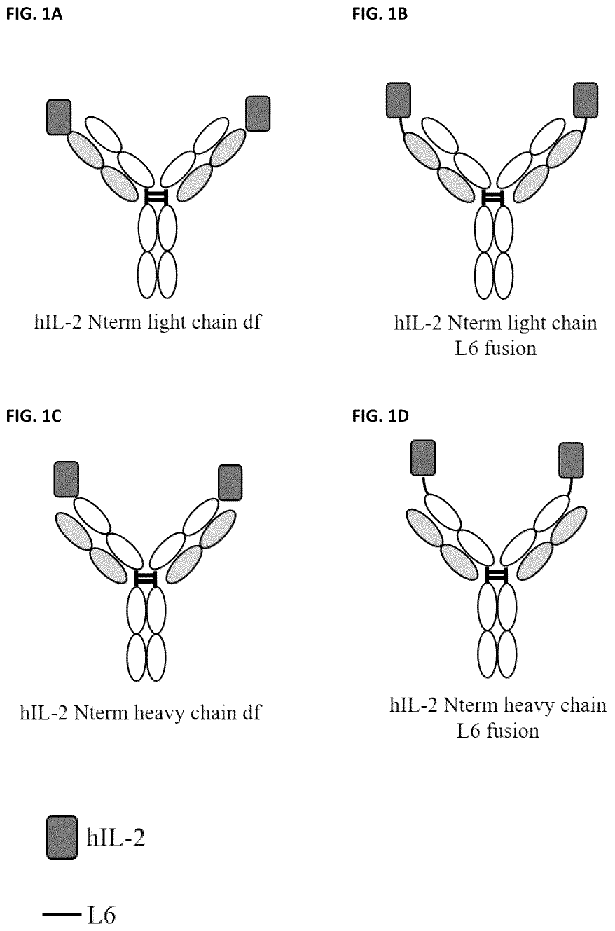

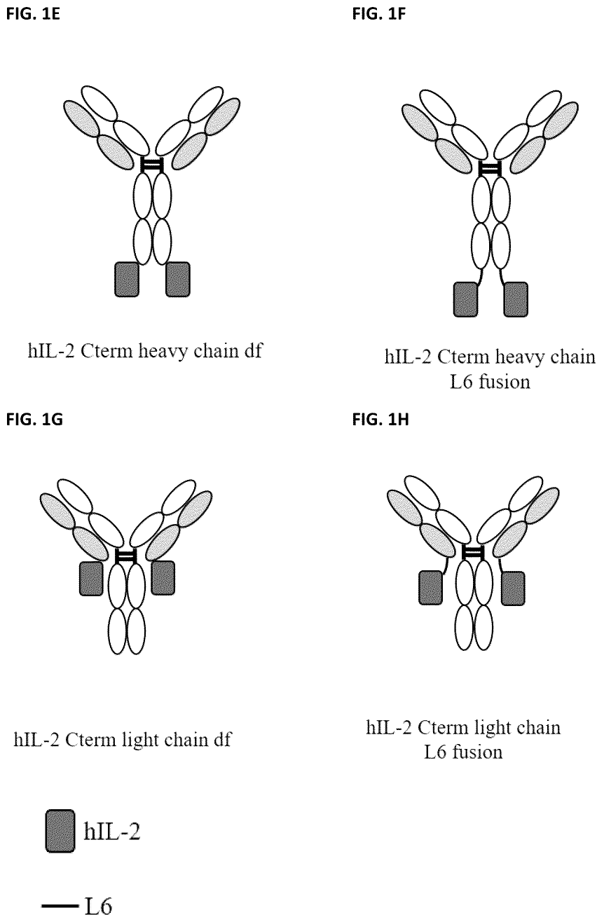

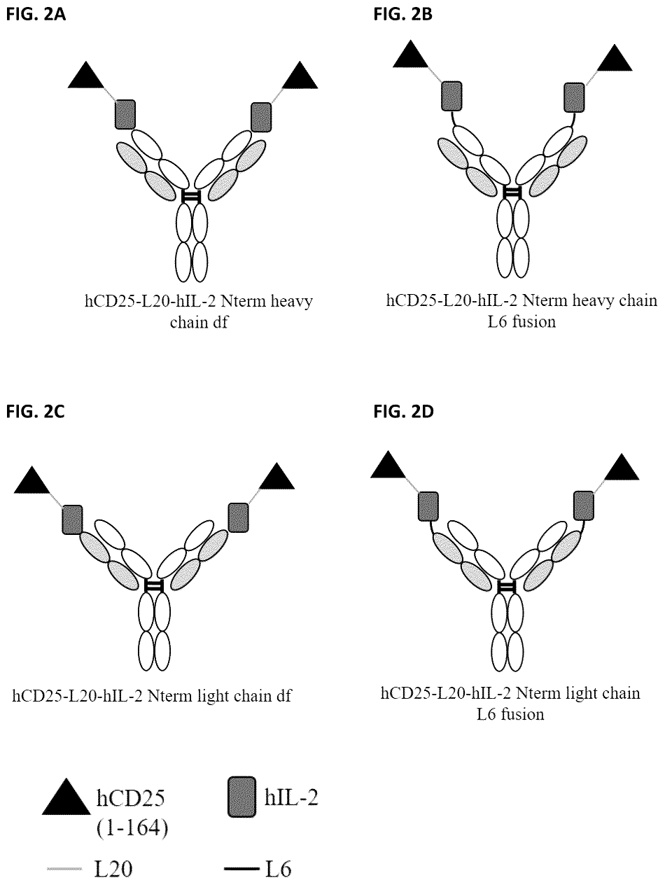

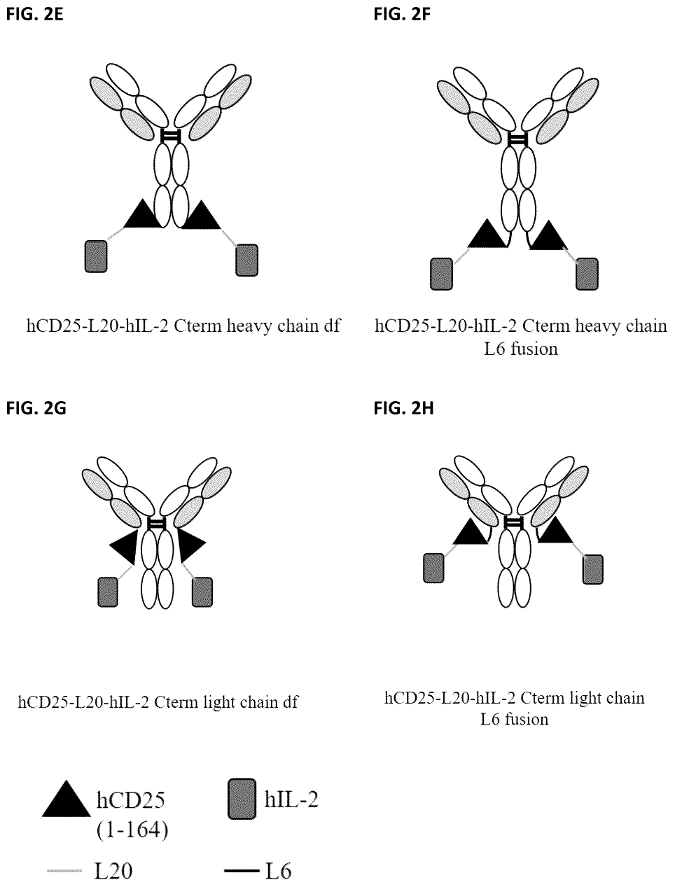

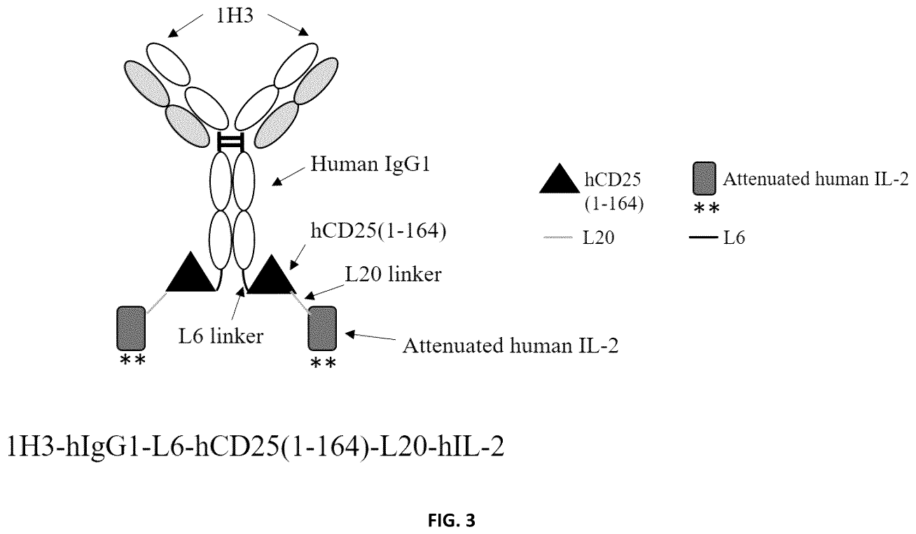

In order to determine the optimal structures for an antibody-attenuated hIL-2 fusion protein, non-attenuated hIL-2 was fused to an anti-DNase I antibody (clone 1H3) designated as 1H3-hIgG1 (SEQ ID NO: 379, SEQ ID NO: 374) in the antibody variable region in a variety of ways as illustrated in FIG. 1. Variations included the hIL-2 fused at the N-terminus of the human anti-DNase I antibody (clone 1H3) immunoglobulin hIgG1 heavy chain or human kappa light chain via a direct fusion (df) denoted as hIL-2 Nterm light chain df (SEQ ID NO: 379, SEQ ID NO: 356), hIL-2 Nterm heavy chain df (SEQ ID NO: 358, SEQ ID NO: 374) or six amino acid linker (L6) (SEQ ID NO: 355) denoted as hIL-2 Nterm light chain L6 fusion (SEQ ID NO: 379, SEQ ID NO: 357) and hIL-2 Nterm heavy chain L6 fusion (SEQ ID NO: 359, SEQ ID NO: 374). Variations in which the hIL-2 moiety was fused to the C-terminus of both the heavy chains and light chains via df or L6 were also created and denoted as hIL-2 Cterm heavy chain df (SEQ ID NO: 360, SEQ ID NO: 374), hIL-2 Cterm heavy chain L6 fusion (SEQ ID NO: 361, SEQ ID: 374), hIL-2 Cterm light chain df (SEQ ID NO:379, SEQ ID NO: 362), hIL-2 Cterm light chain L6 fusion (SEQ ID NO:379, SEQ ID NO: 363). Further variations were generated in which a CD25/IL-2Rα extracellular domain (amino acids 1-164) (SEQ ID NO: 126) was fused to the N-terminus or C-terminus of the heavy chains or the kappa light chains to interfere with the binding of the IL-2 to CD25 of the IL-2 receptor (FIG. 2). In these constructs, human CD25 extracellular domain (amino acids 1-164) (SEQ ID NO: 126) was fused to human IL-2 via a 20 amino acid linker (L20) (SEQ ID NO: 364), which was then directly fused or fused via an L6 linker (SEQ ID NO: 355) to 1H3-hIgG1 heavy chain or light chain at the N terminus: hCD25-L20-hIL-2 Nterm heavy chain df (SEQ ID NO: 365, SEQ ID NO: 374), hCD25-L20-hIL-2 Nterm heavy chain L6 fusion (SEQ ID NO: 366, SEQ ID NO: 374), hCD25-L20-hIL-2 Nterm light chain df (SEQ ID NO: 379, SEQ ID NO: 367), hCD25-L20-hIL-2 Nterm light chain L6 fusion (SEQ ID NO: 379, SEQ ID NO: 368). Lastly, a final set of variants in which the CD25/IL-2Rα extracellular domain moiety (SEQ ID NO: 126) fused to the C-terminus of the heavy chain and kappa light chains were created: hCD25-L20-hIL-2 Cterm heavy chain df (SEQ ID NO: 369, SEQ ID NO: 374), hCD25-L20-hIL-2 Cterm heavy chain L6 fusion (SEQ ID NO: 370, SEQ ID NO: 374), hCD25-L20-hIL-2 Cterm light chain df (SEQ ID NO: 379, SEQ ID NO: 371), hCD25-L20-hIL-2 Cterm light chain L6 fusion (SEQ ID NO: 379, SEQ ID NO:372). These antibody-hIL-2 fusion proteins were produced, expressed, and Protein-A purified using standard techniques. The 16 N- or C-terminus and linker variants described above were evaluated in an in vitro cell-based phosphorylated STAT5 assay using an 8-point, 6-fold serial titration, as described in Protocol D.

Table 1 summarizes the EC₅₀ calculated over the 8-point, 6-fold serially titrated curves using the geometric mean fluorescence intensity (gMFI) calculated by the FlowJo version 10 software. The fold change from rhIL-2 was also calculated for each variant as a measurement of the level of attenuation as compared to the activity of the rhIL-2 positive control. Some EC₅₀ values were unable to be calculated by the GraphPad Prism 7 software and were marked as Not Calculated (NC); however, based on dose-titration curves there was no attenuation for these variants.

Fusions of the hIL-2 moiety to the N-terminus or C-terminus of the immunoglobulin heavy chain resulted in no reduction in IL-2 activity when compared to rhIL-2 on cell lines expressing the high-affinity hIL-2 receptor (NK-92) or intermediate-affinity hIL-2 receptor (TF1+IL-2Rβ). The direct fusion (df) of hIL-2 to the antibody component of the fusion protein resulted in no change in IL-2 activity when compared with fusion employing a six amino acid linker (L6) between the IL-2 and antibody components. Similarly, fusions of the IL-2 component to the heavy chain or light chain of the antibody component resulted in no change in IL-2 activity when compared to rhIL-2. All N- or C-terminus and linker fusion protein variants in which the hCD25/hIL-2Rα moiety was fused to hIL-2 were predicted to exhibit reduced binding of the fusion protein to the CD25 of the hIL-2 receptor on cells. Experimentally these constructs exhibited strongly attenuated hIL-2 activity (by at least 45-fold) on the high affinity IL-2 receptor (NK-92) and by 18-fold on the intermediate hIL-2 receptor (TF1+IL-2Rβ).

TABLE 1
pSTAT5 EC₅₀ and fold change on fusion protein domain variants

| | | | | Attenuation | | | Attenuation |
|---|---|---|---|---|---|---|---|
| HC or LC | HC and | pSTAT5 | Fold change | based on dose- | pSTAT5 | Fold change | based on dose- |
| Component Of | LC SEQ | EC₅₀ | from rhIL-2 | titration curves | EC₅₀ | from rhIL-2 | titration curves |
| Fusion Protein | ID NOs: | (NK-92) | (NK-92) | (NK-92) | (TF1 + IL-2Rβ) | (TF1 + IL-2Rβ) | (TF1 + IL-2RB) |
| hIL-2 Nterm | 358, 374 | <0.1^{a} | 1^{a} | Not Attenuated | 1.11 | 2 | Not Attenuated |
| heavy chain df | | | | | | | |
| hIL-2 Nterm | 359, 374 | NC^{a} | NC^{a} | Not Attenuated | 0.19 | 0 | Not Attenuated |
| heavy chain L6 | | | | | | | |
| fusion | | | | | | | |
| hIL-2 Nterm | 379, 356 | <0.1^{a} | 1^{a} | Not Attenuated | 0.52 | 1 | Not Attenuated |
| light chain df | | | | | | | |
| hIL-2 Nterm | 379, 357 | <0.1^{a} | 0^{a} | Not Attenuated | 0.13 | 0 | Not Attenuated |
| light chain L6 | | | | | | | |
| fusion | | | | | | | |
| hIL-2 Cterm | 360, 374 | <0.1^{a} | 0^{a} | Not Attenuated | <0.1 | 0 | Not Attenuated |
| heavy chain df | | | | | | | |
| hIL-2 Cterm | 361, 374 | NC^{a} | NC^{a} | Not Attenuated | <0.1 | 0 | Not Attenuated |
| heavy chain L6 | | | | | | | |
| fusion | | | | | | | |
| hIL-2 Cterm | 379, 362 | NC^{a} | NC^{a} | Not Attenuated | 1.15 | 1 | Not Attenuated |
| light chain df | | | | | | | |
| hIL-2 Cterm | 379, 363 | <0.1^{a} | 0^{a} | Not Attenuated | 0.25 | 0 | Not Attenuated |
| light chain L6 | | | | | | | |
| fusion | | | | | | | |
| hCD25-L20- | 365, 374 | 7.42 | 1052 | Attenuated | 190.50^{a} | 314^{a} | Attenuated |
| hIL-2 Nterm | | | | | | | |
| heavy chain df | | | | | | | |
| hCD25-L20- | 366, 374 | 2.24 | 318 | Attenuated | 10.70 | 18 | Attenuated |
| hIL-2 Nterm | | | | | | | |
| heavy chain L6 | | | | | | | |
| fusion | | | | | | | |
| hCD25-L20- | 379, 367 | 106.80 | 15149 | Attenuated | 328.50^{a} | 542^{a} | Attenuated |
| hIL-2 Nterm | | | | | | | |
| light chain df | | | | | | | |
| hCD25-L20- | 379, 368 | 4.45 | 631 | Attenuated | 27.88 | 46 | Attenuated |
| hIL-2 Nterm | | | | | | | |
| light chain L6 | | | | | | | |
| fusion | | | | | | | |
| hCD25-L20- | 369, 374 | 1.89 | 149 | Attenuated | 93.89^{a} | 104^{a} | Attenuated |
| hIL-2 Cterm | | | | | | | |
| heavy chain df | | | | | | | |
| hCD25-L20- | 370, 374 | 6.09 | 479 | Attenuated | 90.53^{a} | 101^{a} | Attenuated |
| hIL-2 Cterm | | | | | | | |
| heavy chain L6 | | | | | | | |
| fusion | | | | | | | |
| hCD25-L20- | 379, 371 | 0.58 | 45 | Attenuated | 156.90^{a} | 174^{a} | Attenuated |
| hIL-2 Cterm | | | | | | | |
| light chain df | | | | | | | |
| hCD25-L20- | 379, 372 | 1.76 | 138 | Attenuated | 221.90^{a} | 247^{a} | Attenuated |
| hIL-2 Cterm | | | | | | | |
| light chain L6 | | | | | | | |
| fusion | | | | | | | |

NC = Not Calculated;
^{a}= Fold change is an estimate only since a full four parameter logistic curve was not reached

### Example 2: Antibody-Attenuated hIL-2 Fusion Protein Variant Production and Determination of their Binding Kinetics to Recombinant Human CD25 and/or Human CD122

Since there was no reduction in hIL-2 activity in the various N-terminus or C-terminus immunoglobulin heavy chain fusion proteins, the hIL-2 Cterm heavy chain L6 fusion (SEQ ID NOs: 361, 374), designated as “1H3-hIgG1-L6-hIL-2”, was used as the base construct for antibody-attenuated-hIL-2 fusion protein variants with substitutions in the hIL-2 moiety. Single, double and/or multiple amino acid substitutions were introduced into selected residues of human IL-2 in order to investigate the role those residues play in the recognition of either human CD25/IL-2Rα and/or human CD122/IL-2Rβ or CD132/IL-2Rγ (human IL-2R subunits). Over three hundred antibody-attenuated hIL-2 fusion protein variants with substitutions in the hIL-2 moiety were generated and evaluated in 6 rounds. These variants were first screened using a flow-based phosphorylated STAT5 (pSTAT5) assay at a fixed concentration on IL-2 dependent cell lines (NK-92 and TF1+IL-2Rβ) as well as in dose-titration curves. Phosphorylated STAT5 is a downstream signal of IL-2 activity and was used as a snapshot measurement of IL-2 potency. IL-2 dependent cell proliferation assays were also performed to measure IL-2 activity over a period of 3-4 days. Criteria for attenuated hIL-2 selection included: (1) reduced IL-2 potency on both NK-92 and TF1+IL-2Rβ cell lines with greater than 50% agonist activity on both cell lines; and (2) moderate-to-high production yield.

Human anti-DNase I antibody-hIL-2 fusion proteins were generated by fusing the human IL-2 or the human IL-2 variants (SEQ ID NOs: 1-344, 377, 378, and 575) to the C-terminus of a human anti-DNase I antibody (clone 1H3, having a human IgG1 isotype) heavy chain via the L6 linker, which were combined with the hIgG1 light chain (1H3-hkappa LC; SEQ ID NO: 374) to generate the 1H3-hIgG1-L6-hIL-2 fusion proteins (provided in Table 28). Mouse anti-yellow fever virus antibody-hIL-2 fusion proteins were also generated by fusing human IL-2 variants to the C-terminus of a mouse anti-yellow fever virus antibody (clone 2D12, having a mouse IgG1 isotype) heavy chain with a D265A substitution for decreased immune effector function via the L6 linker, which were combined with the 2D12-mIgG1 light chain (2D12-mKappa LC; SEQ ID NO: 376) to generate the 2D12-mIgG1-D265A-L6-hIL-2 fusion proteins (provided in Table 28). Some of these mouse anti-yellow fever virus antibody-hIL-2 fusion proteins were formatted onto a human IgG1 constant region and were generated in the same manner as described above using, which was combined with 2D12-hKappa light chain (2D12-hKappa LC; SEQ ID NO: 573). Iterations of IL-2 amino acid substitutions were performed in six rounds, designated Groups 1 to 6. 1H3-hIgG1-L6-hIL-2, 2D12-mIgG1-D265A-L6-hIL-2, and 2D12-hIgG1-L6-hIL-2 fusion proteins were produced, expressed, and Protein-A purified using standard techniques.

Group 1 contained an initial series of only 2D12-mIgG1-D265A-L6-hIL-2 or 2D12-hIgG1-L6-hIL-2 fusion proteins which comprised a substitution or combination of substitutions in human IL-2 which were predicted to be involved in binding to only one of the IL-2 receptor subunits CD25/IL-2Rα, CD122/IL-2Rβ, or CD132/IL-2Rγ. The fusion proteins in this group included the following substitutions to IL-2 predicted to modulate binding to CD25/IL-2Rα: F42K (SEQ ID NO: 1), V69A (SEQ ID NO: 2), V69E (SEQ ID NO: 3), V69F (SEQ ID NO: 4), V69G (SEQ ID NO: 5), V69H (SEQ ID NO: 6), V69I (SEQ ID NO: 7), V69K (SEQ ID NO: 8), V69L (SEQ ID NO: 9), V69M (SEQ ID NO: 10), V69Q (SEQ ID NO: 11), V69S (SEQ ID NO: 12), V69T (SEQ ID NO: 13), V69W (SEQ ID NO: 14), V69Y (SEQ ID NO: 15), V69R (SEQ ID NO: 581), (F42K/F44K) (SEQ ID NO: 16), (F44K/Y45R) (SEQ ID NO: 17), (F42K/V69R) (SEQ ID NO: 18), (Y45R/V69R) (SEQ ID NO: 19), (F42K/F44K/Y45R) (SEQ ID NO: 20), (F42A/Y45A/L72G) (SEQ ID NO: 574), (R38A/F42K/Y45R) (SEQ ID NO: 21), (R38E/F42K/Y45R) (SEQ ID NO: 22), (K43E/F42K/Y45R) (SEQ ID NO: 23), (K43T/F42K/Y45R) (SEQ ID NO: 24), (F42K/Y45R/E62A) (SEQ ID NO: 25), (P65R/F42K/Y45R) (SEQ ID NO: 26), (P65S/F42K/Y45R) (SEQ ID NO: 27), (V69A/F42K/Y45R) (SEQ ID NO: 28), (V69D/F42K/Y45R) (SEQ ID NO: 29), or (V69R/F42K/Y45R) (SEQ ID NO: 30). The substitutions in this group included the following substitutions predicted to modulate binding to CD122/IL-2Rβ: D20A (SEQ ID NO: 31), D20N (SEQ ID NO: 32), D20K (SEQ ID NO: 33), N88A (SEQ ID NO: 34), N88G (SEQ ID NO: 35), N88H (SEQ ID NO: 36), N88K (SEQ ID NO: 37), (D20A/D84A) (SEQ ID NO: 38), (D20A/E15A) (SEQ ID NO: 39), (D20A/E95A) (SEQ ID NO: 40), (D20A/N88A) (SEQ ID NO: 41), (D20A/S87A) (SEQ ID NO: 42), (D84A/N88A) (SEQ ID NO: 43), (E15A/N88A) (SEQ ID NO: 44), or (S87A/N88A) (SEQ ID NO: 45). Group 1 also included the following substitutions to IL-2 predicted to modulate IL-2 binding to CD132/IL-2-Ry: Q126L (SEQ ID NO: 377) or Q126E (SEQ ID NO: 378). The IL-2 substitutions studied in Group 1 were not predicted to modulate binding to more than one of the IL-2 receptor subunits.

Group 2 contained a series of 1H3-hIgG1-L6-hIL-2 fusion proteins which comprised one or more substitutions in human IL-2 which were predicted to be involved in CD25/IL-2Rα binding only. The fusion proteins in this group included the following substitutions to IL-2 predicted to modulate binding to CD25/IL-2Rα: R38A (SEQ ID NO: 46), R38D (SEQ ID NO: 47), R38E (SEQ ID NO: 48), R38Q (SEQ ID NO: 49), F42R (SEQ ID NO: 50), F42A (SEQ ID NO: 51), F42D (SEQ ID NO: 52), F42H (SEQ ID NO: 53), K43A (SEQ ID NO: 54), K43E (SEQ ID NO: 55), K43Q (SEQ ID NO: 56), Y45A (SEQ ID NO: 57), Y45K (SEQ ID NO: 58), Y45S (SEQ ID NO: 59), Y45R (SEQ ID NO: 60), E61A (SEQ ID NO: 61), E61R (SEQ ID NO: 62), E61K (SEQ ID NO: 63), E62A (SEQ ID NO: 64), E62R (SEQ ID NO: 65), E62K (SEQ ID NO: 66), E62Y (SEQ ID NO: 67), E68Y (SEQ ID NO: 68), E68A (SEQ ID NO: 69), E68K (SEQ ID NO: 70), E68R (SEQ ID NO: 71), E68L (SEQ ID NO: 72), L72Y (SEQ ID NO: 73), L72R (SEQ ID NO: 74), L72A (SEQ ID NO: 75), L72D (SEQ ID NO: 76), L72H (SEQ ID NO: 77), L72F (SEQ ID NO: 78), (R38D/E61R) (SEQ ID NO: 79), (R38D/E61R/K43E) (SEQ ID NO: 80), or (T3A/F42A/Y45A/L72G/C125A) (SEQ ID NO: 81). The substitution T3A was introduced into the IL-2 amino acid sequence to remove the predicted O-linked glycosylation site on human IL-2 (see for example Int'l Pub. No. WO2012/107417) and the substitution C125A was introduced into the IL-2 amino acid sequence to remove an unpaired cysteine residue (see for example Int'l Pub. No. WO2018/184964). The IL-2 substitutions studied in Group 2 were predicted to not modulate IL-2 binding to CD132/IL-2-Ry, nor were these substitutions predicted to modulate binding to more than one of the IL-2 receptor subunits.

Group 3 contained a series of 1H3-hIgG1-L6-hIL-2 fusion proteins which comprised one or more substitutions in human IL-2 which were predicted to be involved in CD122/IL-2Rβ binding only. The fusion proteins in this group included the following substitutions to IL-2 predicted to modulate binding to CD122/IL-2Rβ: E15A (SEQ ID NO: 82), E15R (SEQ ID NO: 83), E15K (SEQ ID NO: 84), H16A (SEQ ID NO: 85), H16Y (SEQ ID NO: 86), H16E (SEQ ID NO: 87), L19A (SEQ ID NO: 88), D20I (SEQ ID NO: 89), D20S (SEQ ID NO: 90), D20H (SEQ ID NO: 91), D20T (SEQ ID NO: 92), D20W (SEQ ID NO: 93), D20Y (SEQ ID NO: 94), D20R (SEQ ID NO: 95), D20F (SEQ ID NO: 96), R81A (SEQ ID NO: 97), D84A (SEQ ID NO: 98), D84R (SEQ ID NO: 99), D84K (SEQ ID NO: 100), S87A (SEQ ID NO: 101), N88Y (SEQ ID NO: 102), N88D (SEQ ID NO: 103), N88R (SEQ ID NO: 104), N88E (SEQ ID NO: 105), N88F (SEQ ID NO: 106), N88I (SEQ ID NO: 107), 192A (SEQ ID NO: 108), 192Y (SEQ ID NO: 109), 192S (SEQ ID NO: 110), 192F (SEQ ID NO: 111), 192R (SEQ ID NO: 112), 192D (SEQ ID NO: 113), 192E (SEQ ID NO: 114), E95A (SEQ ID NO: 115), E95R (SEQ ID NO: 116), E95K (SEQ ID NO: 117), (D20Y/H16E) (SEQ ID NO: 118), (D20Y/H16A) (SEQ ID NO: 119), (D20Y/H16Y) (SEQ ID NO: 120), (D20Y/192A) (SEQ ID NO: 121), (D20Y/192S) (SEQ ID NO: 122), (D20Y/192R) (SEQ ID NO: 123), (D20Y/E95R) (SEQ ID NO: 124), or (D20Y/E95A) (SEQ ID NO: 125).

Group 4 contained a series of fusion proteins containing the 1H3-hIgG1-L6-hIL-2 HC fused to a CD25/IL-2Rα extracellular domain moiety (SEQ ID NO: 126), a 20 amino acid linker (L20) (SEQ ID NO: 364), and human IL-2 variants comprising one or more substitutions to residues predicted to be involved in binding to CD122/IL-2Rβ. The fusion proteins in this group included the following substitutions to IL-2 predicted to modulate binding to CD122/IL-2Rβ: E15A (SEQ ID NO: 82), D20I (SEQ ID NO: 89), D20S (SEQ ID NO: 90), D20H (SEQ ID NO: 91), D20W (SEQ ID NO: 93), D20Y (SEQ ID NO: 94), D20R (SEQ ID NO: 95), D20F (SEQ ID NO: 96), D84K (SEQ ID NO: 100), S87A (SEQ ID NO: 101), N88Y (SEQ ID NO: 102), N88D (SEQ ID NO: 103), N88R (SEQ ID NO: 104), N88E (SEQ ID NO: 105), N88F (SEQ ID NO: 106), N88I (SEQ ID NO: 107), 192A (SEQ ID NO: 108), E95A (SEQ ID NO: 115), or E95K (SEQ ID NO: 117). The antibody-attenuated hIL-2 fusion proteins in this group are denoted as 1H3-hIgG1-L6-hCD25 (1-164)-L20-hIL-2.

Group 5 contained a series of 1H3-hIgG1-L6-hIL-2 which comprised a combination of substitutions in IL-2 which were predicted to be involved in binding of IL-2 to CD25/IL-2Rα and to CD122/IL-2Rβ or CD132/IL-2Rγ. In addition, some variants had a deletion in the first three amino acids at the N-terminus of the hIL-2 moiety (Δ1-3APT). The fusion proteins in Group 5 included the following substitutions to IL-2 predicted to modulate IL-2 binding to CD25/IL-2Rα and to CD122/IL-2Rβ: (F42D/D20A) (SEQ ID NO: 127), (F42R/D20A) (SEQ ID NO: 128), (F42K/D20A) (SEQ ID NO: 129), (F42A/D20A) (SEQ ID NO: 130), (F42H/D20A) (SEQ ID NO: 131), (Y45R/D20A) (SEQ ID NO: 132), (Y45K/D20A) (SEQ ID NO: 133), (R38N/D20A) (SEQ ID NO: 134), (R38G/D20A) (SEQ ID NO: 135), (R38H/D20A) (SEQ ID NO: 136), (R38I/D20A) (SEQ ID NO: 137), (R38L/D20A) (SEQ ID NO: 138), (R38M/D20A) (SEQ ID NO: 139), (R38F/D20A) (SEQ ID NO: 140), (R38P/D20A) (SEQ ID NO: 141), (R38S/D20A) (SEQ ID NO: 142), (R38T/D20A) (SEQ ID NO: 143), (R38W/D20A) (SEQ ID NO: 144), (R38Y/D20A) (SEQ ID NO: 145), (R38V/D20A) (SEQ ID NO: 146), (R38A/D20A) (SEQ ID NO: 147), (R38Q/D20A) (SEQ ID NO: 148), (D20A/R38E) (SEQ ID NO: 149), (R38D/D20A) (SEQ ID NO: 150), (K43E/D20A) (SEQ ID NO: 151), (E61A/D20A) (SEQ ID NO: 152), (E62A/D20A) (SEQ ID NO: 153), (E62Y/D20A) (SEQ ID NO: 154), (L72D/D20A) (SEQ ID NO: 155), (L72H/D20A) (SEQ ID NO: 156), (L72R/D20A) (SEQ ID NO: 157), (F42D/192D) (SEQ ID NO: 158), (F42R/192D) (SEQ ID NO: 159), (F42H/192D) (SEQ ID NO: 160), (F42A/192D) (SEQ ID NO: 161), (H16A/F42A) (SEQ ID NO: 575), (K43E/192D) (SEQ ID NO: 162), (Y45R/192D) (SEQ ID NO: 163), (Y45K/192D) (SEQ ID NO: 164), (E62A/192D) (SEQ ID NO: 165), (E62Y/192D) (SEQ ID NO: 166), (L72D/192D) (SEQ ID NO: 167), (L72H/192D) (SEQ ID NO: 168), (L72R/192D) (SEQ ID NO: 169), (R38D/192D) (SEQ ID NO: 170), (R38E/192D) (SEQ ID NO: 171), (R38Q/192D) (SEQ ID NO: 172), (R38A/192D) (SEQ ID NO: 173), (R38E/N88R) (SEQ ID NO: 174), (R38E/D84R) (SEQ ID NO: 175), (R38E/D84K) (SEQ ID NO: 176), (F42A/Y45R/D20A) (SEQ ID NO: 177), (F42H/Y45R/D20A) (SEQ ID NO: 178), (R38D/E61R/D20A) (SEQ ID NO: 179), (R38E/E61R/D20A) (SEQ ID NO: 180), (R38Q/E61R/D20A) (SEQ ID NO: 181), (R38A/E61R/D20A) (SEQ ID NO: 182), (R38A/D20A/E95A) (SEQ ID NO: 183), (D20A/E95A/R38D) (SEQ ID NO: 184), (D20A/E95A/R38E) (SEQ ID NO: 185), (D20A/E95A/R38Q) (SEQ ID NO: 186), (D20A/E95A/F42R) (SEQ ID NO: 187), (D20A/E95A/F42A) (SEQ ID NO: 188), (D20A/E95A/F42D) (SEQ ID NO: 189), (D20A/E95A/F42H) (SEQ ID NO: 190), (D20A/E95A/F42K) (SEQ ID NO: 191), (D20A/E95A/K43A) (SEQ ID NO: 192), (D20A/E95A/K43E) (SEQ ID NO: 193), (D20A/E95A/K43Q) (SEQ ID NO: 194), (D20A/E95A/Y45A) (SEQ ID NO: 195), (D20A/E95A/Y45K) (SEQ ID NO: 196), (D20A/E95A/Y45S) (SEQ ID NO: 197), (D20A/E95A/Y45R) (SEQ ID NO: 198), (D20A/E95A/E61A) (SEQ ID NO: 199), (D20A/E95A/E62A) (SEQ ID NO: 200), (D20A/E95A/E62R) (SEQ ID NO: 201), (D20A/E95A/E62K) (SEQ ID NO: 202), (D20A/E95A/E62Y) (SEQ ID NO: 203), (D20A/E95A/E68Y) (SEQ ID NO: 204), (D20A/E95A/E68A) (SEQ ID NO: 205), (D20A/E95A/E68L) (SEQ ID NO: 206), (D20A/E95A/L72Y) (SEQ ID NO: 207), (D20A/E95A/L72R) (SEQ ID NO: 208), (D20A/E95A/L72A) (SEQ ID NO: 209), (D20A/E95A/L72D) (SEQ ID NO: 210), (D20A/E95A/L72H) (SEQ ID NO: 211), (D20A/E95A/L72F) (SEQ ID NO: 212), (F42K/Y45R/D20A/S87A) (SEQ ID NO: 213), (F42K/Y45R/D20A/E95A) (SEQ ID NO: 214), (D20A/R38E/C125A) (SEQ ID NO: 215), (T3A/D20A/R38E) (SEQ ID NO: 216), (T3A/D20A/R38E/C125A) (SEQ ID NO: 217), (A1-3APT/D20A/R38E) (SEQ ID NO: 218), or (A1-3APT/D20A/R38E/C125A) (SEQ ID NO: 219). The fusion proteins in Group 5 included the following substitutions to IL-2 predicted to modulate IL-2 binding to CD25/IL-2Rα and to CD132/IL-2R: (R38E/Q22A) (SEQ ID NO: 220), (R38E/T123A) (SEQ ID NO: 221), (R38E/1129A) (SEQ ID NO: 222), (R38E/S130A) (SEQ ID NO: 223), (R38E/Q126A) (SEQ ID NO: 224), (R38E/Q126D) (SEQ ID NO: 225), (R38E/Q126V) (SEQ ID NO: 226), (R38E/Q22A/S130A) (SEQ ID NO: 227), (F42K/Y45R/Q126D) (SEQ ID NO: 228), or (D20A/E95A/Q126D) (SEQ ID NO: 229). Mutations to the hIL-2 sequence for Group 5 antibody-attenuated hIL-2 fusion proteins in which the numbering is according to IL-2 sequence are listed in SEQ ID NO: 127-229 and 575.

Group 6 contained a series of 1H3-hIgG1-L6-hIL-2 fusion proteins which comprised a combination of substitutions in human IL-2 which were predicted to be involved in binding of IL-2 to CD25/IL-2Rα and to CD122/IL-2Rβ, but not to CD132/IL-2Rγ. The fusion proteins in Group 6 included the following combination of substitutions in IL-2 predicted to modulate IL-2 binding to CD25/IL-2Rα and CD122/IL-2Rβ: (D20A/E61R) (SEQ ID NO: 230), (D20A/E61N) (SEQ ID NO: 231), (D20A/E61D) (SEQ ID NO: 232), (D20A/E61Q) (SEQ ID NO: 233), (D20A/E61G) (SEQ ID NO: 234), (D20A/E61H) (SEQ ID NO: 235), (D20A/E61I) (SEQ ID NO: 236), (D20A/E61L) (SEQ ID NO: 237), (D20A/E61K) (SEQ ID NO: 238), (D20A/E61M) (SEQ ID NO: 239), (D20A/E61F) (SEQ ID NO: 240), (D20A/E61P) (SEQ ID NO: 241), (D20A/E61S) (SEQ ID NO: 242), (D20A/E61T) (SEQ ID NO: 243), (D20A/E61W) (SEQ ID NO: 244), (D20A/E61Y) (SEQ ID NO: 245), (D20A/E61V) (SEQ ID NO: 246), (D20A/F42N) (SEQ ID NO: 247), (D20A/F42Q) (SEQ ID NO: 248), (D20A/F42E) (SEQ ID NO: 249), (D20A F42G) (SEQ ID NO: 250), (D20A/F42I) (SEQ ID NO: 251), (D20A/F42L) (SEQ ID NO: 252), (D20A/F42M) (SEQ ID NO: 253), (D20A/F42P) (SEQ ID NO: 254), (D20A/F42S) (SEQ ID NO: 255), (D20A/F42T) (SEQ ID NO: 256), (D20A/F42W) (SEQ ID NO: 257), (D20A/F42Y) (SEQ ID NO: 258), (D20A/F42V) (SEQ ID NO: 259), (D20A/Y45A) (SEQ ID NO: 260), (D20A/Y45N) (SEQ ID NO: 261), (D20A/Y45D) (SEQ ID NO: 262), (D20A/Y45Q) (SEQ ID NO: 263), (D20A/Y45E) (SEQ ID NO: 264), (D20A/Y45G) (SEQ ID NO: 265), (D20A/Y45H) (SEQ ID NO: 266), (D20A/Y45I) (SEQ ID NO: 267), (D20A/Y45L) (SEQ ID NO: 268), (D20A/Y45M) (SEQ ID NO: 269), (D20A/Y45F) (SEQ ID NO: 270), (D20A/Y45P) (SEQ ID NO: 271), (D20A/Y45S) (SEQ ID NO: 272), (D20A/Y45T) (SEQ ID NO: 273), (D20A/Y45W) (SEQ ID NO: 274), (D20A/Y45V) (SEQ ID NO: 275), (192D/F42N) (SEQ ID NO: 276), (192D/F42Q) (SEQ ID NO: 277), (192D/F42E) (SEQ ID NO: 278), (192D/F42G) (SEQ ID NO: 279), (192D/F42I) (SEQ ID NO: 280), (192D/F42L) (SEQ ID NO: 281), (192D/F42K) (SEQ ID NO: 282), (192D/F42M) (SEQ ID NO: 283), (192D/F42P) (SEQ ID NO: 284), (192D/F42S) (SEQ ID NO: 285), (192D/F42T) (SEQ ID NO: 286), (192D/F42W) (SEQ ID NO: 287), (192D/F42Y) (SEQ ID NO: 288), (192D/F42V) (SEQ ID NO: 289), (192D/Y45A) (SEQ ID NO: 290), (192D/Y45N) (SEQ ID NO: 291), (192D/Y45D) (SEQ ID NO: 292), (192D/Y45Q) (SEQ ID NO: 293), (192D/Y45E) (SEQ ID NO: 294), (192D/Y45G) (SEQ ID NO: 295), (192D/Y45H) (SEQ ID NO: 296), (192D/Y45I) (SEQ ID NO: 297), (192D/Y45L) (SEQ ID NO: 298), (192D/Y45M) (SEQ ID NO: 299), (192D/Y45F) (SEQ ID NO: 300), (192D/Y45P) (SEQ ID NO: 301), (192D/Y45S) (SEQ ID NO: 302), (192D/Y45T) (SEQ ID NO: 303), (192D/Y45W) (SEQ ID NO: 304), (192D/Y45V) (SEQ ID NO: 305), (R38E/D20H) (SEQ ID NO: 306), (R38E/D20S) (SEQ ID NO: 307), (F42A/N88R) (SEQ ID NO: 308), (F42A/N88D) (SEQ ID NO: 309), (R38E/D84A) (SEQ ID NO: 310), (R38E/D84N) (SEQ ID NO: 311), (R38E/D84Q) (SEQ ID NO: 312), (R38E/D84E) (SEQ ID NO: 313), (R38E/D84G) (SEQ ID NO: 314), (R38E/D84H) (SEQ ID NO: 315), (R38E/D84I) (SEQ ID NO: 316), (R38E/D84L) (SEQ ID NO: 317), (R38E/D84M) (SEQ ID NO: 318), (R38E/D84F) (SEQ ID NO: 319), (R38E/D84P) (SEQ ID NO: 320), (R38E/D84S) (SEQ ID NO: 321), (R38E/D84T) (SEQ ID NO: 322), (R38E/D84W) (SEQ ID NO: 323), (R38E/D84Y) (SEQ ID NO: 324), (R38E/D84V) (SEQ ID NO: 325), (R38E/192A) (SEQ ID NO: 326), (R38E/192R) (SEQ ID NO: 327), (R38E/192N) (SEQ ID NO: 328), (R38E/192Q) (SEQ ID NO: 329), (R38E/192E) (SEQ ID NO: 330), (R38E/192G) (SEQ ID NO: 331), (R38E/192H) (SEQ ID NO: 332), (R38E/192L) (SEQ ID NO: 333), (R38E/192K) (SEQ ID NO: 334), (R38E/192M) (SEQ ID NO: 335), (R38E/192F) (SEQ ID NO: 336), (R38E/192P) (SEQ ID NO: 337), (R38E/192S) (SEQ ID NO: 338), (R38E/192T) (SEQ ID NO: 339), (R38E/192W) (SEQ ID NO: 340), (R38E/192Y) (SEQ ID NO: 341), (R38E/192V) (SEQ ID NO: 342), (R38E/H16E) (SEQ ID NO: 343), or (R38K/D20A) (SEQ ID NO: 344). Mutations to the hIL-2 sequence for Group 6 antibody-attenuated hIL-2 fusion proteins in which the numbering is according to IL-2 sequence is listed in SEQ ID NO: 230-344.

The binding kinetics of some purified 1H3-hIgG1-L6-hIL-2 variant proteins for individual recombinant human CD25 and human CD122 were determined using bio-layer interferometry (BLI). Briefly, binding experiments were performed using an Octet Red96 instrument (Pall ForteBio) at 25° C. C-terminal poly-histidine tagged human CD25 and human CD122 extracellular domains were captured onto anti-His2 sensors (Pall ForteBio). Receptor loaded sensors were dipped into a 7-point serial 3-fold dilution of each 1H3-hIgG-L6-hIL-2 variant, starting at a top concentration of 300 nM. 1H3-hIgG1-L6-hIL-2 fusion proteins were diluted into an assay buffer consisting of phosphate buffered saline (PBMS) supplemented with 0.1% BSA, 0.02% Tween-20 (pH 7.2). Loaded sensors were regenerated using 10 mM Glycine buffer (pH 1.7). Kinetic constants were calculated using a monovalent binding model.

Table 2 documents the association constant (kₒₙ), dissociation constant (k_{off}), and equilibrium constant (K_{D}) of 74 immunoglobulin-hIL-2 fusion protein variants bound to recombinant human CD25 or recombinant human CD122.

TABLE 2
Binding kinetics of 1H3-hIgG-L6-hIL-2 fusion proteins
to recombinant human CD25 or CD122 by Octet BLI

| | SEQ ID NO | Predicted receptor | | | |
|---|---|---|---|---|---|
| 1H3-hIgG1-L6-hIL-2 | of hIL-2 | sub-unit targeted | K_{D} | kₒₙ | k_{off} |
| fusion proteins | variant | by IL-2 substitution | (M) | (1/Ms) | (1/s) |
| 1H3-hIgG1-L6-hIL-2 WT | 345 | N/A | 3.20E−10 | 5.90E+05 | 1.90E−04 |
| 1H3-hIgG1-L6-hIL-2 | 82 | CD122 | 2.04E−09 | 1.15E+05 | 2.35E−04 |
| (E15A) | | | | | |
| 1H3-hIgG1-L6-hIL-2 | 83 | CD122 | 3.41E−09 | 9.48E+04 | 3.23E−04 |
| (E15R) | | | | | |
| 1H3-hIgG1-L6-hIL-2 | 84 | CD122 | 1.39E−09 | 1.71E+05 | 2.37E−04 |
| (E15K) | | | | | |
| 1H3-hIgG1-L6-hIL-2 | 85 | CD122 | 1.68E−09 | 1.71E+05 | 2.87E−04 |
| (H16A) | | | | | |
| 1H3-hIgG1-L6-hIL-2 | 86 | CD122 | 1.46E−09 | 1.50E+05 | 2.20E−04 |
| (H16Y) | | | | | |
| 1H3-hIgG1-L6-hIL-2 | 87 | CD122 | 1.40E−09 | 1.57E+05 | 2.20E−04 |
| (H16E) | | | | | |
| 1H3-hIgG1-L6-hIL-2 | 88 | CD122 | 1.76E−09 | 2.03E+05 | 3.57E−04 |
| (L19A) | | | | | |
| 1H3-hIgG1-L6-hIL-2 | 89 | CD122 | 1.16E−09 | 1.70E+05 | 1.98E−04 |
| (D20I) | | | | | |
| 1H3-hIgG1-L6-hIL-2 | 90 | CD122 | 6.24E−10 | 1.74E+05 | 1.09E−04 |
| (D20S) | | | | | |
| 1H3-hIgG1-L6-hIL-2 | 91 | CD122 | 1.13E−09 | 1.88E+05 | 2.12E−04 |
| (D20H) | | | | | |
| 1H3-hIgG1-L6-hIL-2 | 93 | CD122 | 1.01E−09 | 1.87E+05 | 1.90E−04 |
| (D20W) | | | | | |
| 1H3-hIgG1-L6-hIL-2 | 94 | CD122 | 1.42E−09 | 1.51E+05 | 2.14E−04 |
| (D20Y) | | | | | |
| 1H3-hIgG1-L6-hIL-2 | 95 | CD122 | 1.21E−09 | 1.44E+05 | 1.75E−04 |
| (D20R) | | | | | |
| 1H3-hIgG1-L6-hIL-2 | 96 | CD122 | 1.57E−09 | 1.75E+05 | 2.75E−04 |
| (D20F) | | | | | |
| 1H3-hIgG1-L6-hIL-2 | 46 | CD25 | 5.55E−09 | 1.82E+05 | 1.01E−03 |
| (R38A) | | | | | |
| 1H3-hIgG1-L6-hIL-2 | 47 | CD25 | 1.86E−09 | 8.84E+05 | 1.64E−03 |
| (R38D) | | | | | |
| 1H3-hIgG1-L6-hIL-2 | 48 | CD25 | 8.74E−09 | 3.31E+05 | 2.89E−03 |
| (R38E) | | | | | |
| 1H3-hIgG1-L6-hIL-2 | 49 | CD25 | 6.33E−09 | 4.83E+05 | 3.05E−03 |
| (R38Q) | | | | | |
| 1H3-hIgG1-L6-hIL-2 | 50 | CD25 | 2.63E−09 | 1.59E+06 | 4.20E−03 |
| (F42R) | | | | | |
| 1H3-hIgG1-L6-hIL-2 | 51 | CD25 | 9.25E−09 | 9.89E+05 | 9.15E−03 |
| (F42A) | | | | | |
| 1H3-hIgG1-L6-hIL-2 | 52 | CD25 | 4.51E−09 | 1.70E+06 | 7.64E−03 |
| (F42D) | | | | | |
| 1H3-hIgG1-L6-hIL-2 | 53 | CD25 | 6.84E−09 | 8.23E+05 | 5.63E−03 |
| (F42H) | | | | | |
| 1H3-hIgG1-L6-hIL-2 | 54 | CD25 | 4.79E−09 | 2.59E+05 | 1.24E−03 |
| (K43A) | | | | | |
| 1H3-hIgG1-L6-hIL-2 | 55 | CD25 | 5.66E−09 | 4.52E+05 | 2.56E−03 |
| (K43E) | | | | | |
| 1H3-hIgG1-L6-hIL-2 | 56 | CD25 | 2.28E−09 | 4.49E+05 | 1.02E−03 |
| (K43Q) | | | | | |
| 1H3-hIgG1-L6-hIL-2 | 57 | CD25 | 3.66E−09 | 4.29E+05 | 1.57E−03 |
| (Y45A) | | | | | |
| 1H3-hIgG1-L6-hIL-2 | 58 | CD25 | 9.03E−09 | 5.22E+05 | 4.71E−03 |
| (Y45K) | | | | | |
| 1H3-hIgG1-L6-hIL-2 | 59 | CD25 | 2.45E−09 | 5.05E+05 | 1.24E−03 |
| (Y45S) | | | | | |
| 1H3-hIgG1-L6-hIL-2 | 60 | CD25 | 1.96E−09 | 6.46E+05 | 1.27E−03 |
| (Y45R) | | | | | |
| 1H3-hIgG1-L6-hIL-2 | 61 | CD25 | 7.00E−09 | 3.21E+05 | 2.25E−03 |
| (E61A) | | | | | |
| 1H3-hIgG1-L6-hIL-2 | 62 | CD25 | 8.84E−09 | 1.22E+06 | 1.08E−02 |
| (E61R) | | | | | |
| 1H3-hIgG1-L6-hIL-2 | 63 | CD25 | 1.56E−08 | 3.16E+05 | 4.94E−03 |
| (E61K) | | | | | |
| 1H3-hIgG1-L6-hIL-2 | 64 | CD25 | 1.23E−08 | 3.79E+05 | 4.67E−03 |
| (E62A) | | | | | |
| 1H3-hIgG1-L6-hIL-2 | 65 | CD25 | No binding observed | | |
| (E62R) | | | | | |
| 1H3-hIgG1-L6-hIL-2 | 66 | CD25 | No binding observed | | |
| (E62K) | | | | | |
| 1H3-hIgG1-L6-hIL-2 | 67 | CD25 | 1.55E−08 | 2.91E+05 | 4.50E−03 |
| (E62Y) | | | | | |
| 1H3-hIgG1-L6-hIL-2 | 68 | CD25 | 8.18E−09 | 1.80E+05 | 1.47E−03 |
| (E68Y) | | | | | |
| 1H3-hIgG1-L6-hIL-2 | 69 | CD25 | 4.49E−09 | 1.45E+05 | 6.52E−04 |
| (E68A) | | | | | |
| 1H3-hIgG1-L6-hIL-2 | 70 | CD25 | 9.63E−09 | 2.54E+05 | 2.44E−03 |
| (E68K) | | | | | |
| 1H3-hIgG1-L6-hIL-2 | 71 | CD25 | 1.16E−08 | 2.54E+05 | 2.96E−03 |
| (E68R) | | | | | |
| 1H3-hIgG1-L6-hIL-2 | 72 | CD25 | 8.62E−09 | 1.02E+05 | 8.82E−04 |
| (E68L) | | | | | |
| 1H3-hIgG1-L6-hIL-2 | 112 | CD122 | 2.54E−09 | 1.08E+05 | 2.76E−04 |
| (I92R) | | | | | |
| 1H3-hIgG1-L6-hIL-2 | 113 | CD122 | 7.94E−09 | 4.95E+04 | 3.93E−04 |
| (I92D) | | | | | |
| 1H3-hIgG1-L6-hIL-2 | 114 | CD122 | 2.41E−09 | 8.54E+04 | 2.06E−04 |
| (I92E) | | | | | |
| 1H3-hIgG1-L6-hIL-2 | 115 | CD122 | 3.11E−09 | 1.47E+05 | 4.58E−04 |
| (E95A) | | | | | |
| 1H3-hIgG1-L6-hIL-2 | 116 | CD122 | 2.29E−09 | 1.14E+05 | 2.61E−04 |
| (E95R) | | | | | |
| 1H3-hIgG1-L6-hIL-2 | 117 | CD122 | 3.25E−09 | 1.25E+05 | 4.07E−04 |
| (E95K) | | | | | |
| 1H3-hIgG1-L6-hIL-2 | 213 | CD25+CD122 | 3.25E−08 | 1.24E+05 | 4.05E−03 |
| (F42K/Y45R/D20A/S87A) | | | | | |
| 1H3-hIgG1-L6-hIL-2 | 214 | CD25+CD122 | No binding observed | | |
| (F42K/Y45R/D20A/E95A) | | | | | |
| 1H3-hIgG1-L6-hIL-2 | 228 | CD25+CD132 | No binding observed | | |
| (F42K/Y45R/Q126D) | | | | | |
| 1H3-hIgG1-L6-hIL-2 | 229 | CD122+CD132 | 3.34E−09 | 4.87E+04 | 1.62E−04 |
| (D20A/E95A/Q126D) | | | | | |
| 1H3-hIgG1-L6-hIL-2 | 79 | CD25 | No binding observed | | |
| (R38D/E61R) | | | | | |
| 1H3-hIgG1-L6-hIL-2 | 80 | CD25 | No binding observed | | |
| (R38D/E61R/K43E) | | | | | |

Table 3 documents the association (kₒₙ) constants, dissociation (k_{off}) constants, and equilibrium constants (K_{D}) of 74 1H3-hIgG1-L6-hIL-2 fusion proteins bound to recombinant human CD122.

TABLE 3
Binding kinetics of 1H3-hIgG-L6-hIL-2 fusion proteins
to recombinant human CD122 by Octet BLI

| | SEQ ID NO | Predicted receptor | | | |
|---|---|---|---|---|---|
| 1H3-hIgG-L6-hIL-2 | of hIL-2 | sub-unit targeted | K_{D} | kₒₙ | k_{off} |
| fusion proteins | variant | by IL-2 substitution | (M) | (1/Ms) | (1/s) |
| 1H3-hIgG1-L6-hIL-2 WT | 345^{a} | N/A | 5.60E−09 | 7.00E+05 | 3.90E−03 |
| 1H3-hIgG1-L6-hIL-2 | 82^{b} | CD122 | 8.89E−09 | 1.75E+05 | 1.56E−03 |
| (E15A) | | | | | |
| 1H3-hIgG1-L6-hIL-2 | 83 | CD122 | 3.69E−09 | 1.46E+05 | 5.38E−04 |
| (E15R) | | | | | |
| 1H3-hIgG1-L6-hIL-2 | 84 | CD122 | 3.56E−09 | 2.42E+05 | 8.62E−04 |
| (E15K) | | | | | |
| 1H3-hIgG1-L6-hIL-2 | 85 | CD122 | 1.78E−09 | 1.55E+06 | 2.76E−03 |
| (H16A) | | | | | |
| 1H3-hIgG1-L6-hIL-2 | 86 | CD122 | 4.36E−09 | 9.97E+05 | 4.35E−03 |
| (H16Y) | | | | | |
| 1H3-hIgG1-L6-hIL-2 | 87 | CD122 | No binding observed | | |
| (H16E) | | | | | |
| 1H3-hIgG1-L6-hIL-2 | 88 | CD122 | 1.03E−08 | 4.72E+05 | 4.87E−03 |
| (L19A) | | | | | |
| 1H3-hIgG1-L6-hIL-2 | 89 | CD122 | No binding observed | | |
| (D20I) | | | | | |
| 1H3-hIgG1-L6-hIL-2 | 90 | CD122 | No binding observed | | |
| (D20S) | | | | | |
| 1H3-hIgG1-L6-hIL-2 | 91 | CD122 | No binding observed | | |
| (D20H) | | | | | |
| 1H3-hIgG1-L6-hIL-2 | 93 | CD122 | No binding observed | | |
| (D20W) | | | | | |
| 1H3-hIgG1-L6-hIL-2 | 94 | CD122 | No binding observed | | |
| (D20Y) | | | | | |
| 1H3-hIgG1-L6-hIL-2 | 95 | CD122 | No binding observed | | |
| (D20R) | | | | | |
| 1H3-hIgG1-L6-hIL-2 | 96 | CD122 | No binding observed | | |
| (D20F) | | | | | |
| 1H3-hIgG1-L6-hIL-2 | 46 | CD25 | 1.38E−08 | 2.08E+05 | 2.87E−03 |
| (R38A) | | | | | |
| 1H3-hIgG1-L6-hIL-2 | 47 | CD25 | 2.20E−09 | 4.28E+05 | 9.42E−04 |
| (R38D) | | | | | |
| 1H3-hIgG1-L6-hIL-2 | 48 | CD25 | 5.81E−09 | 4.64E+05 | 2.69E−03 |
| (R38E) | | | | | |
| 1H3-hIgG1-L6-hIL-2 | 49 | CD25 | 1.01E−09 | 2.81E+05 | 2.84E−04 |
| (R38Q) | | | | | |
| 1H3-hIgG1-L6-hIL-2 | 50 | CD25 | 5.47E−09 | 5.42E+05 | 2.96E−03 |
| (F42R) | | | | | |
| 1H3-hIgG1-L6-hIL-2 | 51 | CD25 | 5.97E−09 | 4.19E+05 | 2.50E−03 |
| (F42A) | | | | | |
| 1H3-hIgG1-L6-hIL-2 | 52 | CD25 | 1.04E−08 | 2.38E+05 | 2.46E−03 |
| (F42D) | | | | | |
| 1H3-hIgG1-L6-hIL-2 | 53 | CD25 | 6.33E−09 | 4.45E+05 | 2.81E−03 |
| (F42H) | | | | | |
| 1H3-hIgG1-L6-hIL-2 | 54 | CD25 | 1.03E−08 | 2.85E+05 | 2.94E−03 |
| (K43A) | | | | | |
| 1H3-hIgG1-L6-hIL-2 | 55 | CD25 | 5.47E−09 | 3.65E+05 | 2.00E−03 |
| (K43E) | | | | | |
| 1H3-hIgG1-L6-hIL-2 | 56 | CD25 | 4.21E−09 | 5.14E+05 | 2.17E−03 |
| (K43Q) | | | | | |
| 1H3-hIgG1-L6-hIL-2 | 57 | CD25 | 4.93E−09 | 4.51E+05 | 2.22E−03 |
| (Y45A) | | | | | |
| 1H3-hIgG1-L6-hIL-2 | 58 | CD25 | 6.56E−09 | 3.55E+05 | 2.33E−03 |
| (Y45K) | | | | | |
| 1H3-hIgG1-L6-hIL-2 | 59 | CD25 | 6.96E−09 | 5.07E+05 | 3.53E−03 |
| (Y45S) | | | | | |
| 1H3-hIgG1-L6-hIL-2 | 60 | CD25 | 7.58E−09 | 4.36E+05 | 3.31E−03 |
| (Y45R) | | | | | |
| 1H3-hIgG1-L6-hIL-2 | 61 | CD25 | 1.34E−08 | 3.13E+05 | 4.18E−03 |
| (E61A) | | | | | |
| 1H3-hIgG1-L6-hIL-2 | 62 | CD25 | 1.30E−08 | 4.56E+05 | 5.91E−03 |
| (E61R) | | | | | |
| 1H3-hIgG1-L6-hIL-2 | 63 | CD25 | 1.56E−08 | 3.16E+05 | 4.94E−03 |
| (E61K) | | | | | |
| 1H3-hIgG1-L6-hIL-2 | 64 | CD25 | 1.23E−08 | 3.79E+05 | 4.67E−03 |
| (E62A) | | | | | |
| 1H3-hIgG1-L6-hIL-2 | 65 | CD25 | No binding observed | | |
| (E62R) | | | | | |
| 1H3-hIgG1-L6-hIL-2 | 66 | CD25 | No binding observed | | |
| (E62K) | | | | | |
| 1H3-hIgG1-L6-hIL-2 | 67 | CD25 | 1.55E−08 | 2.91E+05 | 4.50E−03 |
| (E62Y) | | | | | |
| 1H3-hIgG1-L6-hIL-2 | 68 | CD25 | 8.18E−09 | 1.80E+05 | 1.47E−03 |
| (E68Y) | | | | | |
| 1H3-hIgG1-L6-hIL-2 | 69 | CD25 | 4.49E−09 | 1.45E+05 | 6.52E−04 |
| (E68A) | | | | | |
| 1H3-hIgG1-L6-hIL-2 | 70 | CD25 | 1.05E−08 | 2.02E+05 | 2.12E−03 |
| (E68K) | | | | | |
| 1H3-hIgG1-L6-hIL-2 | 71 | CD25 | 8.51E−09 | 2.27E+05 | 1.93E−03 |
| (E68R) | | | | | |
| 1H3-hIgG1-L6-hIL-2 | 72 | CD25 | 2.72E−09 | 7.79E+04 | 2.12E−04 |
| (E68L) | | | | | |
| 1H3-hIgG1-L6-hIL-2 | 73 | CD25 | 6.39E−09 | 1.94E+05 | 1.24E−03 |
| (L72Y) | | | | | |
| 1H3-hIgG1-L6-hIL-2 | 74 | CD25 | 1.96E−08 | 3.07E+04 | 6.01E−04 |
| (L72R) | | | | | |
| 1H3-hIgG1-L6-hIL-2 | 75 | CD25 | 9.08E−09 | 1.47E+05 | 1.34E−03 |
| (L72A) | | | | | |
| 1H3-hIgG1-L6-hIL-2 | 76 | CD25 | 9.52E−09 | 1.57E+05 | 1.50E−03 |
| (L72D) | | | | | |
| 1H3-hIgG1-L6-hIL-2 | 77 | CD25 | 9.03E−09 | 1.65E+05 | 1.49E−03 |
| (L72H) | | | | | |
| 1H3-hIgG1-L6-hIL-2 | 78 | CD25 | 5.04E−09 | 2.28E+05 | 1.15E−03 |
| (L72F) | | | | | |
| 1H3-hIgG1-L6-hIL-2 | 97 | CD122 | 7.08E−09 | 2.20E+05 | 1.56E−03 |
| (R81A) | | | | | |
| 1H3-hIgG1-L6-hIL-2 | 98 | CD122 | No binding observed | | |
| (D84A) | | | | | |
| 1H3-hIgG1-L6-hIL-2 | 99 | CD122 | 1.88E−08 | 4.73E+05 | 8.88E−03 |
| (D84R) | | | | | |
| 1H3-hIgG1-L6-hIL-2 | 101 | CD122 | 7.09E−09 | 3.31E+05 | 2.34E−03 |
| (S87A) | | | | | |
| 1H3-hIgG1-L6-hIL-2 | 102 | CD122 | No binding observed | | |
| (N88Y) | | | | | |
| 1H3-hIgG1-L6-hIL-2 | 103 | CD122 | No binding observed | | |
| (N88D) | | | | | |
| 1H3-hIgG1-L6-hIL-2 | 104 | CD122 | No binding observed | | |
| (N88R) | | | | | |
| 1H3-hIgG1-L6-hIL-2 | 105 | CD122 | No binding observed | | |
| (N88E) | | | | | |
| 1H3-hIgG1-L6-hIL-2 | 106 | CD122 | No binding observed | | |
| (N88F) | | | | | |
| 1H3-hIgG1-L6-hIL-2 | 107 | CD122 | No binding observed | | |
| (N88I) | | | | | |
| 1H3-hIgG1-L6-hIL-2 | 108 | CD122 | 3.38E−09 | 1.95E+06 | 6.58E−03 |
| (I92A) | | | | | |
| 1H3-hIgG1-L6-hIL-2 | 109 | CD122 | 1.23E−08 | 4.53E+05 | 5.57E−03 |
| (I92Y) | | | | | |
| 1H3-hIgG1-L6-hIL-2 | 110 | CD122 | No binding observed | | |
| (I92S) | | | | | |
| 1H3-hIgG1-L6-hIL-2 | 111 | CD122 | 5.45E−09 | 1.03E+05 | 5.59E−04 |
| (I92F) | | | | | |
| 1H3-hIgG1-L6-hIL-2 | 112 | CD122 | No binding observed | | |
| (I92R) | | | | | |
| 1H3-hIgG1-L6-hIL-2 | 113 | CD122 | No binding observed | | |
| (I92D) | | | | | |
| 1H3-hIgG1-L6-hIL-2 | 114 | CD122 | 1.62E−09 | 9.33E+05 | 1.51E−03 |
| (I92E) | | | | | |
| 1H3-hIgG1-L6-hIL-2 | 115 | CD122 | 8.17E−09 | 2.87E+05 | 2.35E−03 |
| (E95A) | | | | | |
| 1H3-hIgG1-L6-hIL-2 | 116 | CD122 | No binding observed | | |
| (E95R) | | | | | |
| 1H3-hIgG1-L6-hIL-2 | 117 | CD122 | 3.81E−09 | 6.58E+05 | 2.51E−03 |
| (E95K) | | | | | |
| 1H3-hIgG1-L6-hIL-2 | 213 | CD25+CD122 | No binding observed | | |
| (F42K/Y45R/D20A/S87A) | | | | | |
| 1H3-hIgG1-L6-hIL-2 | 214 | CD25+CD122 | No binding observed | | |
| (F42K/Y45R/D20A/E95A) | | | | | |
| 1H3-hIgG1-L6-hIL-2 | 228 | CD25+CD132 | 9.16E−09 | 3.75E+05 | 3.44E−03 |
| (F42K/Y45R/Q126D) | | | | | |
| 1H3-hIgG1-L6-hIL-2 | 229 | CD122+CD132 | No binding observed | | |
| (D20A/E95A/Q126D) | | | | | |
| 1H3-hIgG1-L6-hIL-2 | 79 | CD25 | 1.29E−08 | 3.68E+05 | 4.73E−03 |
| (R38D/E61R) | | | | | |
| 1H3-hIgG1-L6-hIL-2 | 80 | CD25 | 7.47E−09 | 4.52E+05 | 3.38E−03 |
| (R38D/E61R/K43E) | | | | | |

^{a}SEQ ID NO: 345 corresponds to wild type hIL-2.
^{b}SEQ ID NO: 57 is attenuated IL-2 sequence only.

### Example 3: Testing for Attenuation for the High-Affinity and Intermediate-Affinity hIL-2 Receptor with a Fixed Concentration Cell-Based Potency pSTAT5 Screen

The attenuation of antibody-attenuated hIL-2 fusion proteins described in Example 2 was tested in a fixed concentration pSTAT5 screen using the NK-92 (expressing the high affinity hIL-2 receptor) and TF1+IL-2Rβ (expressing the intermediate affinity hIL-2 receptor) cell lines as described in Protocol D. Tables 4-8 list the fold change of geometric mean fluorescent intensity (gMFI) of antibody-attenuated hIL-2 fusion proteins from free cytokine wild-type rhIL-2, a measurement of reduction of IL-2 activity. For the fixed concentration screen, the fold change was calculated by dividing the gMFI of the rhIL-2 by the gMFI of the variants. For experiments with full titration curves, fold change from rhIL-2 was calculated by dividing the EC₅₀ values for the rhIL-2 by the EC₅₀ of variants. Fold change was rounded to the nearest whole number. A reduced gMFI in both NK-92 and TF1+IL-2Rβ cell lines when compared to the gMFI resulting from rhIL-2 was indicative of attenuation of IL-2 activity at both the high- and intermediate-affinity receptors. Group 1 variants described in Example 2 were not tested in the fixed concentration cell-based potency pSTAT5 screen.

Each variant tested was also assessed for IL-2 agonistic activity and characterized either as a full or partial IL-2 agonist, or having no IL-2 activity (inactive). 1H3-hIgG1-L6-hIL-2 fusion protein dose-titration curves that reached the maximal gMFI level exhibited by the rhIL-2 positive control were considered to be antibody-attenuated hIL-2 fusion protein with full agonist activity. Partial agonist activity was calculated as a percentage of full activity using rhIL-2 maximal gMFI as 100%. Antibody-attenuated hIL-2 fusion protein with less than 10% of the rhIL-2 maximal gMFI at the highest concentration of 1200 nM were considered to have no agonist activity (inactive). Some EC₅₀ values and level of attenuation could not be accurately calculated using the GraphPad Prism 7 software since activity did not reach a maximum and accordingly these values are an estimate.

pSTAT5 fixed concentration results demonstrated that while some single residue substitutions attenuated IL-2 activity on the high-affinity cell line (NK-92), a combination of substitutions which modulated binding to both the alpha chain and the beta chain or both the alpha chain and gamma chain were required to substantially attenuate IL-2 activity on the high affinity IL-2 receptor (more than 20-fold attenuation from recombinant hIL-2).

TABLE 4
Fold change from rhIL-2 in a fixed concentration pSTAT5
screen on 1H3-hIgG1-L6-hIL-2 fusion proteins from Group 2

| | | Fold | Fold |
|---|---|---|---|
| | SEQ ID | change | change |
| | NO of | from | from hIL-2 |
| | hIL-2 | hIL-2 | (TF1 + IL − |
| Variants | variant | (NK-92) | 2Rβ) |
| 1H3-hIgG1-L6-hIL-2 (R38D) | 47 | 7 | 1 |
| 1H3-hIgG1-L6-hIL-2 (R38E) | 48 | 12 | 1 |
| 1H3-hIgG1-L6-hIL-2 (R38Q) | 49 | 1 | 1 |
| 1H3-hIgG1-L6-hIL-2 (F42R) | 50 | 7 | 1 |
| 1H3-hIgG1-L6-hIL-2 (F42A) | 51 | 1 | 1 |
| 1H3-hIgG1-L6-hIL-2 (F42H) | 53 | 1 | 1 |
| 1H3-hIgG1-L6-hIL-2 (K43E) | 55 | 1 | 1 |
| 1H3-hIgG1-L6-hIL-2 (K43Q) | 56 | 1 | 1 |
| 1H3-hIgG1-L6-hIL-2 (Y45A) | 57 | 1 | 1 |
| 1H3-hIgG1-L6-hIL-2 (Y45K) | 58 | 1 | 1 |
| 1H3-hIgG1-L6-hIL-2 (Y45S) | 59 | 1 | 1 |
| 1H3-hIgG1-L6-hIL-2 (Y45R) | 60 | 10 | 1 |
| 1H3-hIgG1-L6-hIL-2 (E68Y) | 68 | 1 | 1 |
| 1H3-hIgG1-L6-hIL-2 (E68A) | 69 | 1 | 1 |
| 1H3-hIgG1-L6-hIL-2 (E68L) | 72 | 2 | 1 |
| 1H3-hIgG1-L6-hIL-2 (L72Y) | 73 | 1 | 1 |
| 1H3-hIgG1-L6-hIL-2 (L72A) | 75 | 1 | 1 |
| 1H3-hIgG1-L6-hIL-2 (L72F) | 78 | 1 | 1 |
| 1H3-hIgG1-L6-hIL-2 | 79 | NT-1 | 1 |
| (R38D/E61R) | | | |
| 1H3-hIgG1-L6-hIL-2 | 80 | NT-1 | 1 |
| (R38D/E61R/K43E) | | | |
| 1H3-hIgG1-L6-hIL-2 | 81 | 10 | 1 |
| (T3A/F42A/Y45A/L72G/C125A) | | | |

NT-1 = Already tested in pSTAT5 full titration first, no data for fixed concentration assay.

TABLE 5
Fold change from rhIL-2 in a fixed concentration pSTAT5
screen on 1H3-hIgG1-L6-hIL-2 fusion proteins from Group 3

| | | Fold | Fold |
|---|---|---|---|
| | SEQ ID | change | change |
| | NO of | from | from hIL-2 |
| | hIL-2 | hIL-2 | (TF1 + IL − |
| Variants | variant | (NK-92) | 2Rβ) |
| 1H3-hIgG1-L6-hIL-2 (E15A) | 82 | 1 | 1 |
| 1H3-hIgG1-L6-hIL-2 (E15R) | 83 | 1 | 1 |
| 1H3-hIgG1-L6-hIL-2 (E15K) | 84 | 1 | 1 |
| 1H3-hIgG1-L6-hIL-2 (H16A) | 85 | 1 | 1 |
| 1H3-hIgG1-L6-hIL-2 (H16Y) | 86 | 1 | 1 |
| 1H3-hIgG1-L6-hIL-2 (H16E) | 87 | 1 | NT-1 |
| 1H3-hIgG1-L6-hIL-2 (L19A) | 88 | 1 | NT-1 |
| 1H3-hIgG1-L6-hIL-2 (D20I) | 89 | 12 | NT-1 |
| 1H3-hIgG1-L6-hIL-2 (D20S) | 90 | 4 | NT-1 |
| 1H3-hIgG1-L6-hIL-2 (D20H) | 91 | 10 | NT-1 |
| 1H3-hIgG1-L6-hIL-2 (D20W) | 93 | 16 | NT-1 |
| 1H3-hIgG1-L6-hIL-2 (D20Y) | 94 | 17 | NT-1 |
| 1H3-hIgG1-L6-hIL-2 (D20R) | 95 | 19 | NT-1 |
| 1H3-hIgG1-L6-hIL-2 (D20F) | 96 | 17 | NT-1 |
| 1H3-hIgG1-L6-hIL-2 (R81A) | 97 | 1 | 1 |
| 1H3-hIgG1-L6-hIL-2 (D84A) | 98 | 1 | NT-1 |
| 1H3-hIgG1-L6-hIL-2 (D84R) | 99 | 3 | NT-1 |
| 1H3-hIgG1-L6-hIL-2 (D84K) | 100 | 2 | NT-1 |
| 1H3-hIgG1-L6-hIL-2 (S87A) | 101 | 1 | 1 |
| 1H3-hIgG1-L6-hIL-2 (N88Y) | 102 | 22 | NT-1 |
| 1H3-hIgG1-L6-hIL-2 (N88D) | 103 | 2 | NT-1 |
| 1H3-hIgG1-L6-hIL-2 (N88R) | 104 | 3 | NT-1 |
| 1H3-hIgG1-L6-hIL-2 (N88E) | 105 | 10 | NT-1 |
| 1H3-hIgG1-L6-hIL-2 (N88F) | 106 | 19 | NT-1 |
| 1H3-hIgG1-L6-hIL-2 (N88I) | 107 | 9 | NT-1 |
| 1H3-hIgG1-L6-hIL-2 (I92A) | 108 | 1 | 1 |
| 1H3-hIgG1-L6-hIL-2 (I92Y) | 109 | 1 | NT-1 |
| 1H3-hIgG1-L6-hIL-2 (I92S) | 110 | 1 | NT-1 |
| 1H3-hIgG1-L6-hIL-2 (I92F) | 111 | 1 | 1 |
| 1H3-hIgG1-L6-hIL-2 (I92R) | 112 | 1 | NT-1 |
| 1H3-hIgG1-L6-hIL-2 (I92D) | 113 | 3 | NT-1 |
| 1H3-hIgG1-L6-hIL-2 (I92E) | 114 | 1 | 1 |
| 1H3-hIgG1-L6-hIL-2 (E95A) | 115 | 1 | 1 |
| 1H3-hIgG1-L6-hIL-2 (E95R) | 116 | 1 | NT-1 |
| 1H3-hIgG1-L6-hIL-2 (E95K) | 117 | 1 | 1 |
| 1H3-hIgG1-L6-hIL-2 (D20T) | 92 | 3 | 2 |
| 1H3-hIgG1-L6-hIL-2 (D20A) | 31 | 7 | 2 |
| 1H3-hIgG1-L6-hIL-2 | 118 | 6 | 4 |
| (D20Y/H16E) | | | |
| 1H3-hIgG1-L6-hIL-2 | 119 | 10 | 3 |
| (D20Y/H16A) | | | |
| 1H3-hIgG1-L6-hIL-2 | 120 | 10 | 4 |
| (D20Y/H16Y) | | | |
| 1H3-hIgG1-L6-hIL-2 (D20Y/I92A) | 121 | 11 | 4 |
| 1H3-hIgG1-L6-hIL-2 (D20Y/I92S) | 122 | 11 | 4 |
| 1H3-hIgG1-L6-hIL-2 (D20Y/I92R) | 123 | 12 | 5 |
| 1H3-hIgG1-L6-hIL-2 | 124 | 12 | 5 |
| (D20Y/E95R) | | | |
| 1H3-hIgG1-L6-hIL-2 | 125 | 11 | 5 |
| (D20Y/E95A) | | | |

NT-1 = Already tested in pSTAT5 full titration first, no data for fixed concentration assay.

TABLE 6
Fold change from rhIL-2 in a fixed concentration pSTAT5
screen on 1H3-hIgG1-L6-hIL-2 fusion proteins from Group 4

| | SEQ | Fold | Fold change |
|---|---|---|---|
| | ID | change | from hIL- |
| | NO of | from | 2(TF1 + |
| | hIL-2 | hIL-2 | IL − |
| Variants | variant | (NK-92) | 2Rβ) |
| 1H3-hIgG1-L6-hCD25(1-164)-L20-hIL-2 | 82 | 17 | 1 |
| (E15A) | | | |
| 1H3-hIgG1-L6-hCD25(1-164)-L20-hIL-2 | 89 | 21 | 15 |
| (D20I) | | | |
| 1H3-hIgG1-L6-hCD25(1-164)-L20-hIL-2 | 90 | 2 | 14 |
| (D20S) | | | |
| 1H3-hIgG1-L6-hCD25(1-164)-L20-hIL-2 | 91 | 22 | 15 |
| (D20H) | | | |
| 1H3-hIgG1-L6-hCD25(1-164)-L20-hIL-2 | 93 | 23 | 15 |
| (D20W) | | | |
| 1H3-hIgG1-L6-hCD25(1-164)-L20-hIL-2 | 94 | 23 | 16 |
| (D20Y) | | | |
| 1H3-hIgG1-L6-hCD25(1-164)-L20-hIL-2 | 95 | 23 | 16 |
| (D20R) | | | |
| 1H3-hIgG1-L6-hCD25(1-164)-L20-hIL-2 | 96 | 23 | 18 |
| (D20F) | | | |
| 1H3-hIgG1-L6-hCD25(1-164)-L20-hIL-2 | 100 | 23 | 17 |
| (D84K) | | | |
| 1H3-hIgG1-L6-hCD25(1-164)-L20-hIL-2 | 101 | 17 | 2 |
| (S87A) | | | |
| 1H3-hIgG1-L6-hCD25(1-164)-L20-hIL-2 | 102 | 23 | 18 |
| (N88Y) | | | |
| 1H3-hIgG1-L6-hCD25(1-164)-L20-hIL-2 | 103 | 23 | 17 |
| (N88D) | | | |
| 1H3-hIgG1-L6-hCD25(1-164)-L20-hIL-2 | 104 | 24 | 17 |
| (N88R) | | | |
| 1H3-hIgG1-L6-hCD25(1-164)-L20-hIL-2 | 105 | 25 | 18 |
| (N88E) | | | |
| 1H3-hIgG1-L6-hCD25(1-164)-L20-hIL-2 | 106 | 25 | 18 |
| (N88F) | | | |
| 1H3-hIgG1-L6-hCD25(1-164)-L20-hIL-2 | 107 | 25 | 20 |
| (N88I) | | | |
| 1H3-hIgG1-L6-hCD25(1-164)-L20-hIL-2 | 108 | 15 | 3 |
| (192A) | | | |
| 1H3-hIgG1-L6-hCD25(1-164)-L20-hIL-2 | 115 | 14 | 1 |
| (E95A) | | | |
| 1H3-hIgG1-L6-hCD25(1-164)-L20-hIL-2 | 117 | 25 | 8 |
| (E95K) | | | |

TABLE 7
Fold change from rhIL-2 in a fixed concentration pSTAT5
screen on 1H3-hIgG1-L6-hIL-2 fusion proteins from Group 5

| | SEQ ID | Fold change | Fold change |
|---|---|---|---|
| | NO of hIL- | from hIL-2 | from hIL-2 |
| Variants | 2 variant | (NK-92) | (TF1 + IL − 2Rβ) |
| 1H3-hIgG1-L6-hIL-2 (F42D/D20A) | 127 | 11 | 3 |
| 1H3-hIgG1-L6-hIL-2 (F42R/D20A) | 128 | 10 | 2 |
| 1H3-hIgG1-L6-hIL-2 (F42K/D20A) | 129 | 10 | 4 |
| 1H3-hIgG1-L6-hIL-2 (F42A/D20A) | 130 | 11 | 3 |
| 1H3-hIgG1-L6-hIL-2 (F42H/D20A) | 131 | 12 | 2 |
| 1H3-hIgG1-L6-hIL-2 (Y45R/D20A) | 132 | 11 | 1 |
| 1H3-hIgG1-L6-hIL-2 (Y45K/D20A) | 133 | 11 | 2 |
| 1H3-hIgG1-L6-hIL-2 (R38N/D20A) | 134 | 14 | 3 |
| 1H3-hIgG1-L6-hIL-2 (R38G/D20A) | 135 | 13 | 2 |
| 1H3-hIgG1-L6-hIL-2 (R38H/D20A) | 136 | 12 | 3 |
| 1H3-hIgG1-L6-hIL-2 (R38I/D20A) | 137 | 11 | 3 |
| 1H3-hIgG1-L6-hIL-2 (R38L/D20A) | 138 | 11 | 4 |
| 1H3-hIgG1-L6-hIL-2 (R38M/D20A) | 139 | 11 | 3 |
| 1H3-hIgG1-L6-hIL-2 (R38F/D20A) | 140 | 12 | 3 |
| 1H3-hIgG1-L6-hIL-2 (R38P/D20A) | 141 | 13 | 3 |
| 1H3-hIgG1-L6-hIL-2 (R38S/D20A) | 142 | 15 | 2 |
| 1H3-hIgG1-L6-hIL-2 (R38T/D20A) | 143 | 13 | 3 |
| 1H3-hIgG1-L6-hIL-2 (R38W/D20A) | 144 | 14 | 2 |
| 1H3-hIgG1-L6-hIL-2 (R38Y/D20A) | 145 | 14 | 3 |
| 1H3-hIgG1-L6-hIL-2 (R38V/D20A) | 146 | 12 | 3 |
| 1H3-hIgG1-L6-hIL-2 (R38A/D20A) | 147 | 13 | 3 |
| 1H3-hIgG1-L6-hIL-2 (R38Q/D20A) | 148 | 14 | 3 |
| 1H3-hIgG1-L6-hIL-2 (R38E/D20A) | 149 | 15 | 3 |
| 1H3-hIgG1-L6-hIL-2 (R38D/D20A) | 150 | 15 | 3 |
| 1H3-hIgG1-L6-hIL-2 (K43E/D20A) | 151 | 13 | 2 |
| 1H3-hIgG1-L6-hIL-2(E61A/D20A) | 152 | 14 | 2 |
| 1H3-hIgG1-L6-hIL-2 (E62A/D20A) | 153 | 14 | 2 |
| 1H3-hIgG1-L6-hIL-2 (E62Y/D20A) | 154 | 14 | 3 |
| 1H3-hIgG1-L6-hIL-2 (L72D/D20A) | 155 | 14 | 2 |
| 1H3-hIgG1-L6-hIL-2 (L72H/D20A) | 156 | 14 | 3 |
| 1H3-hIgG1-L6-hIL-2 (L72R/D20A) | 157 | 10 | 3 |
| 1H3-hIgG1-L6-hIL-2 (F42D/I92D) | 158 | 12 | 5 |
| 1H3-hIgG1-L6-hIL-2 (F42R/I92D) | 159 | 12 | 3 |
| 1H3-hIgG1-L6-hIL-2 (F42H/I92D) | 160 | 12 | 2 |
| 1H3-hIgG1-L6-hIL-2 (F42A/I92D) | 161 | 12 | 3 |
| 1H3-hIgG1-L6-hIL-2 (K43E/I92D) | 162 | 13 | 4 |
| 1H3-hIgG1-L6-hIL-2 (Y45R/I92D) | 163 | 13 | 1 |
| 1H3-hIgG1-L6-hIL-2 (Y45K/I92D) | 164 | 13 | 1 |
| 1H3-hIgG1-L6-hIL-2 (E62A/I92D) | 165 | 13 | 3 |
| 1H3-hIgG1-L6-hIL-2(E62Y/I92D) | 166 | 14 | 5 |
| 1H3-hIgG1-L6-hIL-2 (L72D/I92D) | 167 | 14 | 5 |
| 1H3-hIgG1-L6-hIL-2 (L72H/I92D) | 168 | 14 | 5 |
| 1H3-hIgG1-L6-hIL-2 (L72R/I92D) | 169 | 14 | 5 |
| 1H3-hIgG1-L6-hIL-2 (R38D/I92D) | 170 | 15 | 2 |
| 1H3-hIgG1-L6-hIL-2 (R38E/I92D) | 171 | 15 | 2 |
| 1H3-hIgG1-L6-hIL-2 (R38Q/I92D) | 172 | 14 | 2 |
| 1H3-hIgG1-L6-hIL-2 (R38A/I92D) | 173 | 13 | 4 |
| 1H3-hIgG1-L6-hIL-2 (R38E/N88R) | 174 | 16 | 1 |
| 1H3-hIgG1-L6-hIL-2 (R38E/D84R) | 175 | 14 | 2 |
| 1H3-hIgG1-L6-hIL-2 (R38E/D84K) | 176 | 14 | 2 |
| 1H3-hIgG1-L6-hIL-2 (F42A/Y45R/D20A) | 177 | 11 | 2 |
| 1H3-hIgG1-L6-hIL-2 (F42H/Y45R/D20A) | 178 | 12 | 2 |
| 1H3-hIgG1-L6-hIL-2 (R38D/E61R/D20A) | 179 | 12 | 3 |
| 1H3-hIgG1-L6-hIL-2 (R38E/E61R/D20A) | 180 | 11 | 3 |
| 1H3-hIgG1-L6-hIL-2 (R38Q/E61R/D20A) | 181 | 12 | 3 |
| 1H3-hIgG1-L6-hIL-2 (R38A/E61R/D20A) | 182 | 13 | 3 |
| 1H3-hIgG1-L6-hIL-2 (D20A/E95A/R38A) | 183 | 6 | 5 |
| 1H3-hIgG1-L6-hIL-2 (D20A/E95A/R38D) | 184 | 21 | 6 |
| 1H3-hIgG1-L6-hIL-2 (D20A/E95A/R38E) | 185 | 21 | 5 |
| 1H3-hIgG1-L6-hIL-2 (D20A/E95A/R38Q) | 186 | 19 | 6 |
| 1H3-hIgG1-L6-hIL-2 (D20A/E95A/F42R) | 187 | 21 | 4 |
| 1H3-hIgG1-L6-hIL-2 (D20A/E95A/F42A) | 188 | 21 | 5 |
| 1H3-hIgG1-L6-hIL-2 (D20A/E95A/F42D) | 189 | 22 | 12 |
| 1H3-hIgG1-L6-hIL-2 (D20A/E95A/F42H) | 190 | 22 | 4 |
| 1H3-hIgG1-L6-hIL-2 (D20A/E95A/F42K) | 191 | 21 | 4 |
| 1H3-hIgG1-L6-hIL-2 (D20A/E95A/K43A) | 192 | 5 | 7 |
| 1H3-hIgG1-L6-hIL-2 (D20A/E95A/K43E) | 193 | 13 | 6 |
| 1H3-hIgG1-L6-hIL-2 (D20A/E95A/K43Q) | 194 | 5 | 6 |
| 1H3-hIgG1-L6-hIL-2 (D20A/E95A/Y45A) | 195 | 4 | 5 |
| 1H3-hIgG1-L6-hIL-2 (D20A/E95A/Y45K) | 196 | 22 | 6 |
| 1H3-hIgG1-L6-hIL-2 (D20A/E95A/Y45S) | 197 | 5 | 6 |
| 1H3-hIgG1-L6-hIL-2 (D20A/E95A/Y45R) | 198 | 25 | 6 |
| 1H3-hIgG1-L6-hIL-2 (D20A/E95A/E61A) | 199 | 10 | 6 |
| 1H3-hIgG1-L6-hIL-2 (D20A/E95A/E62A) | 200 | 23 | 6 |
| 1H3-hIgG1-L6-hIL-2 (D20A/E95A/E62R) | 201 | 25 | 17 |
| 1H3-hIgG1-L6-hIL-2 (D20A/E95A/E62K) | 202 | 25 | 15 |
| 1H3-hIgG1-L6-hIL-2 (D20A/E95A/E62Y) | 203 | 25 | 10 |
| 1H3-hIgG1-L6-hIL-2 (D20A/E95A/E68Y) | 204 | 8 | 5 |
| 1H3-hIgG1-L6-hIL-2 (D20A/E95A/E68A) | 205 | 5 | 8 |
| 1H3-hIgG1-L6-hIL-2(D20A/E95A/E68L) | 206 | 7 | 9 |
| 1H3-hIgG1-L6-hIL-2 (D20A/E95A/L72Y) | 207 | 1 | 5 |
| 1H3-hIgG1-L6-hIL-2 (D20A/E95A/L72R) | 208 | 12 | 9 |
| 1H3-hIgG1-L6-hIL-2 (D20A/E95A/L72A) | 209 | 2 | 6 |
| 1H3-hIgG1-L6-hIL-2 (D20A/E95A/L72D) | 210 | 21 | 6 |
| 1H3-hIgG1-L6-hIL-2 (D20A/E95A/L72H) | 211 | 14 | 6 |
| 1H3-hIgG1-L6-hIL-2 (D20A/E95A/L72F) | 212 | 2 | 6 |
| 1H3-hIgG1-L6-hIL-2 | 214 | 21 | 5 |
| (D20A/E95A/F42K/Y45R) | | | |
| 1H3-hIgG1-L6-hIL-2 | 215 | 16 | 3 |
| (D20A/R38E/C125A) | | | |
| 1H3-hIgG1-L6-hIL-2 (T3A/D20A/R38E) | 216 | 18 | 2 |
| 1H3-hIgG1-L6-hIL-2 | 217 | 18 | 3 |
| (T3A/D20A/R38E/C125A) | | | |
| 1H3-hIgG1-L6-hIL-2 (Δ1- | 218 | 13 | 1 |
| 3APT/D20A/R38E) | | | |
| 1H3-hIgG1-L6-hIL-2 (Δ1- | 219 | 15 | 4 |
| 3APT/D20A/R38E/C125A) | | | |
| 1H3-hIgG1-L6-hIL-2 (R38E/Q22A) | 220 | 12 | 0 |
| 1H3-hIgG1-L6-hIL-2 (R38E/T123A) | 221 | 12 | 0 |
| 1H3-hIgG1-L6-hIL-2 (R38E/I129A) | 222 | 13 | 1 |
| 1H3-hIgG1-L6-hIL-2 (R38E/S130A) | 223 | 12 | 0 |
| 1H3-hIgG1-L6-hIL-2 (R38E/Q126A) | 224 | 13 | 1 |
| 1H3-hIgG1-L6-hIL-2 (R38E/Q126D) | 225 | 15 | 4 |
| 1H3-hIgG1-L6-hIL-2 (R38E/Q126V) | 226 | 14 | 1 |
| 1H3-hIgG1-L6-hIL-2 | 227 | 13 | 1 |
| (R38E/Q22A/S130A) | | | |

TABLE 8
Fold change from rhIL-2 in a fixed concentration pSTAT5
screen on 1H3-hIgG1-L6-hIL-2 fusion proteins from Group 6

| | SEQ ID NO | Fold change | Fold change |
|---|---|---|---|
| | of hIL-2 | from hIL-2 | from hIL-2 |
| Variants | variant | (NK-92) | (TF1 + IL − 2Rβ) |
| 1H3-hIgG1-L6-hIL-2 | 230 | 25 | 4 |
| (D20A/E61R) | | | |
| 1H3-hIgG1-L6-hIL-2 | 231 | 15 | 1 |
| (D20A/E61N) | | | |
| 1H3-hIgG1-L6-hIL-2 | 232 | 11 | 0 |
| (D20A/E61D) | | | |
| 1H3-hIgG1-L6-hIL-2 | 233 | 16 | 2 |
| (D20A/E61Q) | | | |
| 1H3-hIgG1-L6-hIL-2 | 234 | 15 | 2 |
| (D20A/E61G) | | | |
| 1H3-hIgG1-L6-hIL-2 | 235 | 15 | 2 |
| (D20A/E61H) | | | |
| 1H3-hIgG1-L6-hIL-2 (D20A/E61I) | 236 | 16 | 1 |
| 1H3-hIgG1-L6-hIL-2 | 237 | 16 | 1 |
| (D20A/E61L) | | | |
| 1H3-hIgG1-L6-hIL-2 | 238 | 17 | 2 |
| (D20A/E61K) | | | |
| 1H3-hIgG1-L6-hIL-2 | 239 | 15 | 2 |
| (D20A/E61M) | | | |
| 1H3-hIgG1-L6-hIL-2 (D20A/E61F) | 240 | 14 | 0 |
| 1H3-hIgG1-L6-hIL-2 (D20A/E61P) | 241 | 15 | 1 |
| 1H3-hIgG1-L6-hIL-2 (D20A/E61S) | 242 | 16 | 1 |
| 1H3-hIgG1-L6-hIL-2 | 243 | 16 | 2 |
| (D20A/E61T) | | | |
| 1H3-hIgG1-L6-hIL-2 | 244 | 15 | 2 |
| (D20A/E61W) | | | |
| 1H3-hIgG1-L6-hIL-2 | 245 | 15 | 2 |
| (D20A/E61Y) | | | |
| 1H3-hIgG1-L6-hIL-2 | 246 | 17 | 3 |
| (D20A/E61V) | | | |
| 1H3-hIgG1-L6-hIL-2 | 247 | 15 | 2 |
| (D20A/F42N) | | | |
| 1H3-hIgG1-L6-hIL-2 | 248 | 15 | 2 |
| (D20A/F42Q) | | | |
| 1H3-hIgG1-L6-hIL-2 (D20A/F42E) | 249 | 16 | 1 |
| 1H3-hIgG1-L6-hIL-2 | 250 | 17 | 2 |
| (D20A/F42G) | | | |
| 1H3-hIgG1-L6-hIL-2 (D20A/F42I) | 251 | 17 | 2 |
| 1H3-hIgG1-L6-hIL-2 (D20A/F42L) | 252 | 14 | 2 |
| 1H3-hIgG1-L6-hIL-2 | 253 | 15 | 2 |
| (D20A/F42M) | | | |
| 1H3-hIgG1-L6-hIL-2 (D20A/F42P) | 254 | 17 | 2 |
| 1H3-hIgG1-L6-hIL-2 (D20A/F42S) | 255 | 15 | 2 |
| 1H3-hIgG1-L6-hIL-2 (D20A/F42T) | 256 | 16 | 2 |
| 1H3-hIgG1-L6-hIL-2 | 257 | 16 | 2 |
| (D20A/F42W) | | | |
| 1H3-hIgG1-L6-hIL-2 | 258 | 17 | 2 |
| (D20A/F42Y) | | | |
| 1H3-hIgG1-L6-hIL-2 | 259 | 18 | 2 |
| (D20A/F42V) | | | |
| 1H3-hIgG1-L6-hIL-2 | 260 | 15 | 2 |
| (D20A/Y45A) | | | |
| 1H3-hIgG1-L6-hIL-2 | 261 | 14 | 2 |
| (D20A/Y45N) | | | |
| 1H3-hIgG1-L6-hIL-2 | 262 | 18 | 3 |
| (D20A/Y45D) | | | |
| 1H3-hIgG1-L6-hIL-2 | 263 | 17 | 3 |
| (D20A/Y45Q) | | | |
| 1H3-hIgG1-L6-hIL-2 | 264 | 18 | 3 |
| (D20A/Y45E) | | | |
| 1H3-hIgG1-L6-hIL-2 | 265 | 18 | 2 |
| (D20A/Y45G) | | | |
| 1H3-hIgG1-L6-hIL-2 | 266 | 16 | 2 |
| (D20A/Y45H) | | | |
| 1H3-hIgG1-L6-hIL-2 (D20A/Y45I) | 267 | 13 | 2 |
| 1H3-hIgG1-L6-hIL-2 | 268 | 13 | 2 |
| (D20A/Y45L) | | | |
| 1H3-hIgG1-L6-hIL-2 | 269 | 16 | 3 |
| (D20A/Y45M) | | | |
| 1H3-hIgG1-L6-hIL-2 | 270 | 13 | 2 |
| (D20A/Y45F) | | | |
| 1H3-hIgG1-L6-hIL-2 | 271 | 25 | 4 |
| (D20A/Y45P) | | | |
| 1H3-hIgG1-L6-hIL-2 | 272 | 14 | 3 |
| (D20A/Y45S) | | | |
| 1H3-hIgG1-L6-hIL-2 | 273 | 24 | 3 |
| (D20A/Y45T) | | | |
| 1H3-hIgG1-L6-hIL-2 | 274 | 19 | 3 |
| (D20A/Y45W) | | | |
| 1H3-hIgG1-L6-hIL-2 | 275 | 21 | 3 |
| (D20A/Y45V) | | | |
| 1H3-hIgG1-L6-hIL-2 (I92D/F42N) | 276 | 29 | 5 |
| 1H3-hIgG1-L6-hIL-2 (I92D/F42Q) | 277 | 29 | 4 |
| 1H3-hIgG1-L6-hIL-2 (I92D/F42E) | 278 | 30 | 5 |
| 1H3-hIgG1-L6-hIL-2 (I92D/F42G) | 279 | 32 | 5 |
| 1H3-hIgG1-L6-hIL-2 (I92D/F42I) | 280 | 31 | 4 |
| 1H3-hIgG1-L6-hIL-2 (I92D/F42L) | 281 | 31 | 5 |
| 1H3-hIgG1-L6-hIL-2 (I92D/F42K) | 282 | 26 | 4 |
| 1H3-hIgG1-L6-hIL-2 (I92D/F42M) | 283 | 28 | 5 |
| 1H3-hIgG1-L6-hIL-2 (I92D/F42P) | 284 | 29 | 4 |
| 1H3-hIgG1-L6-hIL-2 (I92D/F42S) | 285 | 30 | 5 |
| 1H3-hIgG1-L6-hIL-2 (I92D/F42T) | 286 | 28 | 3 |
| 1H3-hIgG1-L6-hIL-2 (I92D/F42W) | 287 | 18 | 4 |
| 1H3-hIgG1-L6-hIL-2 (I92D/F42Y) | 288 | 22 | 3 |
| 1H3-hIgG1-L6-hIL-2 (I92D/F42V) | 289 | 30 | 3 |
| 1H3-hIgG1-L6-hIL-2 (I92D/Y45A) | 290 | 11 | 3 |
| 1H3-hIgG1-L6-hIL-2 (I92D/Y45N) | 291 | 4 | 3 |
| 1H3-hIgG1-L6-hIL-2 (I92D/Y45D) | 292 | 29 | 5 |
| 1H3-hIgG1-L6-hIL-2 (I92D/Y45Q) | 293 | 25 | 3 |
| 1H3-hIgG1-L6-hIL-2 (I92D/Y45E) | 294 | 27 | 4 |
| 1H3-hIgG1-L6-hIL-2 (I92D/Y45G) | 295 | 20 | 3 |
| 1H3-hIgG1-L6-hIL-2 (I92D/Y45H) | 296 | 7 | 3 |
| 1H3-hIgG1-L6-hIL-2 (I92D/Y45I) | 297 | 20 | 4 |
| 1H3-hIgG1-L6-hIL-2 (I92D/Y45L) | 298 | 5 | 3 |
| 1H3-hIgG1-L6-hIL-2 | 299 | 14 | 3 |
| (I92D/Y45M) | | | |
| 1H3-hIgG1-L6-hIL-2 (I92D/Y45F) | 300 | 10 | 3 |
| 1H3-hIgG1-L6-hIL-2 (I92D/Y45P) | 301 | 28 | 4 |
| 1H3-hIgG1-L6-hIL-2 (I92D/Y45S) | 302 | 11 | 3 |
| 1H3-hIgG1-L6-hIL-2 (I92D/Y45T) | 303 | 28 | 4 |
| 1H3-hIgG1-L6-hIL-2 | 304 | 27 | 4 |
| (I92D/Y45W) | | | |
| 1H3-hIgG1-L6-hIL-2 (I92D/Y45V) | 305 | 28 | 5 |
| 1H3-hIgG1-L6-hIL-2 | 306 | 17 | 5 |
| (R38E/D20H) | | | |
| 1H3-hIgG1-L6-hIL-2 (R38E/D20S) | 307 | 17 | 3 |
| 1H3-hIgG1-L6-hIL-2 | 308 | 18 | 3 |
| (F42A/N88R) | | | |
| 1H3-hIgG1-L6-hIL-2 | 309 | 18 | 3 |
| (F42A/N88D) | | | |
| 1H3-hIgG1-L6-hIL-2 | 310 | 18 | 1 |
| (R38E/D84A) | | | |
| 1H3-hIgG1-L6-hIL-2 | 311 | 18 | 1 |
| (R38E/D84N) | | | |
| 1H3-hIgG1-L6-hIL-2 | 312 | 18 | 1 |
| (R38E/D84Q) | | | |
| 1H3-hIgG1-L6-hIL-2(R38E/D84E) | 313 | 16 | 1 |
| 1H3-hIgG1-L6-hIL-2 | 314 | 18 | 1 |
| (R38E/D84G) | | | |
| 1H3-hIgG1-L6-hIL-2 | 315 | 19 | 2 |
| (R38E/D84H) | | | |
| 1H3-hIgG1-L6-hIL-2 (R38E/D84I) | 316 | 20 | 1 |
| 1H3-hIgG1-L6-hIL-2(R38E/D84L) | 317 | 19 | 1 |
| 1H3-hIgG1-L6-hIL-2 | 318 | 20 | 2 |
| (R38E/D84M) | | | |
| 1H3-hIgG1-L6-hIL-2 (R38E/D84F) | 319 | 20 | 2 |
| 1H3-hIgG1-L6-hIL-2 (R38E/D84P) | 320 | 20 | 1 |
| 1H3-hIgG1-L6-hIL-2 (R38E/D84S) | 321 | 21 | 1 |
| 1H3-hIgG1-L6-hIL-2 (R38E/D84T) | 322 | 20 | 1 |
| 1H3-hIgG1-L6-hIL-2 | 323 | 21 | 1 |
| (R38E/D84W) | | | |
| 1H3-hIgG1-L6-hIL-2 | 324 | 21 | 1 |
| (R38E/D84Y) | | | |
| 1H3-hIgG1-L6-hIL-2 | 325 | 22 | 1 |
| (R38E/D84V) | | | |
| 1H3-hIgG1-L6-hIL-2 (R38E/I92A) | 326 | 22 | 1 |
| 1H3-hIgG1-L6-hIL-2 (R38E/I92R) | 327 | 22 | 2 |
| 1H3-hIgG1-L6-hIL-2 (R38E/I92N) | 328 | 22 | 1 |
| 1H3-hIgG1-L6-hIL-2 (R38E/I92Q) | 329 | 22 | 1 |
| 1H3-hIgG1-L6-hIL-2 (R38E/I92E) | 330 | 22 | 2 |
| 1H3-hIgG1-L6-hIL-2 (R38E/I92G) | 331 | 22 | 1 |
| 1H3-hIgG1-L6-hIL-2 (R38E/I92H) | 332 | 21 | 1 |
| 1H3-hIgG1-L6-hIL-2 (R38E/I92L) | 333 | 17 | 1 |
| 1H3-hIgG1-L6-hIL-2 (R38E/I92K) | 334 | 24 | 3 |
| 1H3-hIgG1-L6-hIL-2 (R38E/I92M) | 335 | 20 | 1 |
| 1H3-hIgG1-L6-hIL-2 (R38E/I92F) | 336 | 16 | 1 |
| 1H3-hIgG1-L6-hIL-2 (R38E/I92P) | 337 | 24 | 5 |
| 1H3-hIgG1-L6-hIL-2 (R38E/I92S) | 338 | 22 | 2 |
| 1H3-hIgG1-L6-hIL-2 (R38E/I92T) | 339 | 22 | 1 |
| 1H3-hIgG1-L6-hIL-2 (R38E/I92W) | 340 | 23 | 1 |
| 1H3-hIgG1-L6-hIL-2 (R38E/I92Y) | 341 | 21 | 1 |
| 1H3-hIgG1-L6-hIL-2 (R38E/I92V) | 342 | 21 | 1 |
| 1H3-hIgG1-L6-hIL-2 (R38E/H16E) | 343 | 25 | 3 |
| 1H3-hIgG1-L6-hIL-2 | 344 | 17 | 3 |
| (R38K/D20A) | | | |

### Example 4: Testing for Attenuation of IL-2 Fusion Proteins for Each of the High-Affinity and Intermediate-Affinity hIL-2 Receptors with a Cell-Based Potency pSTAT5 Dose-Titration Screen

The attenuation of selected antibody-attenuated hIL-2 fusion proteins described in Example 2 (1H3-hIgG1-L6-hIL-2 fusion protein from Groups 2-6) were tested in pSTAT5 titration curves using the NK-92 and TF1+IL-2Rβ cell lines as described in Protocol D.

The gMFI of the Alexa Fluor 647 pSTAT5-positive signal was used to generate four parameter logistic curves and GraphPad Prism 7 software was then used to calculate EC₅₀ values. These values were compared to recombinant hIL-2 (rhIL-2) control as a measurement of attenuation. Tables 9-13 summarize the fold change in activity from rhIL-2 calculated using the gMFI of the Alexa Fluor 647 signal.

An increase in the fold change from rhIL-2 was indicative of the degree of attenuation of hIL-2 activity. Each antibody-attenuated hIL-2 fusion protein tested in the pSTAT5 titration curve was also assessed for agonistic activity and characterized as either full, partial, or no activity (inactive). Antibody-attenuated hIL-2 fusion protein dose-titration curves that reached the maximal gMFI level as the rhIL-2 were considered to be variants with full agonist activity. Partial agonist activity was calculated as described in Example 3. Inactive antibody-attenuated hIL-2 fusion proteins were classified as having less than 10% activity in comparison to rhIL-2. Some fold changes from rhIL-2 could not be accurately calculated (denoted as Not Calculated or “NC”) using the GraphPad Prism 7 software since a full four parameter logistic curve was not generated and accordingly these values are an estimate (annotated as ª in Tables 9-13). However, these variants had greater than 10,000-fold attenuation from rhIL-2 on graphs (data not shown). This is denoted on Tables 9-13 as “>10,000 on graph; NC”.

Full titration pSTAT5 curves demonstrated similar findings as presented in Example 3 in which substitutions that modulated binding to both the alpha chain and the beta chain substantially attenuated IL-2 activity on the high affinity IL-2 receptor in comparison to single substitutions for binding to the alpha or beta chain only. The full titration pSTAT5 assay was additionally able to differentiate between variants with substitutions that caused inactivity versus highly attenuated variants. Finally, comparison of dose-titration curves illustrated more accurate of levels of attenuation over a fixed concentration assay.

TABLE 9
Fold change from rhIL-2 and agonistic activity on
1H3-hIgG1-L6-hIL-2 fusion proteins from Group 2

| | | | | Fold | |
|---|---|---|---|---|---|
| | SEQ | Fold | | change | Agonistic |
| | ID NO | change | | from | Activity |
| | of hIL- | from | Agonistic | rhIL-2 | (TF1 + |
| | 2 | rhIL-2 | Activity | (TF1 + IL − | IL − |
| Variants | variant | (NK-92) | (NK-92) | 2Rβ) | 2Rβ) |
| 1H3-hIgG1-L6-hIL-2 (R38A) | 46 | 2 | Full | 0 | Full |
| 1H3-hIgG1-L6-hIL-2 (R38D) | 47 | 55 | Full | 1 | Full |
| 1H3-hIgG1-L6-hIL-2 (R38E) | 48 | 99-136 | Full | 0 | Full |
| 1H3-hIgG1-L6-hIL-2 (F42R) | 50 | 65 | Full | 0 | Full |
| 1H3-hIgG1-L6-hIL-2 (F42D) | 52 | 193 | Full | 0 | Full |
| 1H3-hIgG1-L6-hIL-2 (K43A) | 54 | 3 ^{a} | Full | 0 | Full |
| 1H3-hIgG1-L6-hIL-2 (Y45R) | 57 | 81 ^{a} | Full | 0 | Full |
| 1H3-hIgG1-L6-hIL-2 (E61A) | 61 | 3 ^{a} | Full | 0 | Full |
| 1H3-hIgG1-L6-hIL-2 (E61R) | 62 | 22 ^{a} | Full | 0 | Full |
| 1H3-hIgG1-L6-hIL-2 (E61K) | 63 | 14 ^{a} | Full | 0 | Full |
| 1H3-hIgG1-L6-hIL-2 (E62A) | 64 | 6 ^{a} | Full | 0 | Full |
| 1H3-hIgG1-L6-hIL-2 (E62R) | 65 | >10,000 | Partial, | 38 | Full |
| | | | 80% | | |
| 1H3-hIgG1-L6-hIL-2 (E62K) | 66 | 2048 | Full | 10 | Full |
| 1H3-hIgG1-L6-hIL-2 (E62Y) | 67 | 18 | Full | 0 | Full |
| 1H3-hIgG1-L6-hIL-2 (E68K) | 70 | 2 ^{a} | Full | 0 | Full |
| 1H3-hIgG1-L6-hIL-2 (E68R) | 71 | 3 ^{a} | Full | 0 | Full |
| 1H3-hIgG1-L6-hIL-2 (L72R) | 74 | 2 ^{a} | Full | 1 | Full |
| 1H3-hIgG1-L6-hIL-2 (L72D) | 76 | 4 ^{a} | Full | 0 | Full |
| 1H3-hIgG1-L6-hIL-2 (L72H) | 77 | 1 ^{a} | Partial, | 0 | Full |
| | | | 80% | | |
| 1H3-hIgG1-L6-hIL-2 | 79 | 479 | Partial, | NT | NT |
| (R38D/E61R) | | | 80% | | |
| 1H3-hIgG1-L6-hIL-2 | 80 | 598 | Full | NT | NT |
| (R38D/E61R/K43E) | | | | | |
| 1H3-hIgG1-L6-hIL-2 | 81 | 360- | Full | 0 | Full |
| (T3A/F42A/Y45A/L72G/C125A) | | 1426 | | | |

NT = not tested
^{a} = Fold change is an estimate only since a full four parameter logistic curve was not reached

TABLE 10
Fold change from rhIL-2 and agonistic activity on 1H3-
hIgG1-L6-hIL-2 fusion proteins variants from Group 3

| | | | | Fold | |
|---|---|---|---|---|---|
| | | Fold | | change | |
| | SEQ ID | change | | from | Agonistic |
| | NO of | from | Agonistic | rhIL-2 | Activity |
| | hIL-2 | rhIL-2 | Activity | (TF1 + IL − | (TF1 + IL − |
| Variants | variant | (NK-92) | (NK-92) | 2Rβ) | 2Rβ) |
| 1H3-hIgG1-L6-hIL-2 | 87 | 63 | Full | 63 | Full |
| (H16E) | | | | | |
| 1H3-hIgG1-L6-hIL-2 | 88 | 0 | Full | 0 | Full |
| (L19A) | | | | | |
| 1H3-hIgG1-L6-hIL-2 | 89 | NT | NT | >10,000 | Inactive |
| (D20I) | | | | on graph, | |
| | | | | NC ^{a} | |
| 1H3-hIgG1-L6-hIL-2 | 90 | 28 | Partial, | 277 ^{a} | Partial, 80% |
| (D20S) | | | 80% | | |
| 1H3-hIgG1-L6-hIL-2 | 91 | >10,000 ^{a} | Partial, | 2767 ^{a} | Partial, 50% |
| (D20H) | | | 90% | | |
| 1H3-hIgG1-L6-hIL-2 | 93 | NT | NT | >10,000 | Inactive |
| (D20W) | | | | on graph, | |
| | | | | NC ^{a} | |
| 1H3-hIgG1-L6-hIL-2 | 94 | >10,000 ^{a} | Partial, | 84-143 ^{a} | Partial, 80% |
| (D20Y) | | | 50-70% | | |
| 1H3-hIgG1-L6-hIL-2 | 95 | NT | NT | >10,000 | Inactive |
| (D20R) | | | | on graph, | |
| | | | | NC ^{a} | |
| 1H3-hIgG1-L6-hIL-2 | 96 | NT | NT | >10,000 | Inactive |
| (D20F) | | | | on graph, | |
| | | | | NC ^{a} | |
| 1H3-hIgG1-L6-hIL-2 | 98 | NT | NT | 14 | Full |
| (D84A) | | | | | |
| 1H3-hIgG1-L6-hIL-2 | 99 | 16 | Full | 244 ^{a} | Partial, 70% |
| (D84R) | | | | | |
| 1H3-hIgG1-L6-hIL-2 | 100 | 14 | Partial, | 195 ^{a} | Full |
| (D84K) | | | 90% | | |
| 1H3-hIgG1-L6-hIL-2 | 102 | NT | NT | >10,000 | Inactive |
| (N88Y) | | | | on graph, | |
| | | | | NC ^{a} | |
| 1H3-hIgG1-L6-hIL-2 | 103 | 21 | Partial, | 130 | Full |
| (N88D) | | | 90% | | |
| 1H3-hIgG1-L6-hIL-2 | 104 | 5-27 | Partial, | 289-556 | Partial, 40% |
| (N88R) | | | 80%-Full | | |
| 1H3-hIgG1-L6-hIL-2 | 105 | NT | NT | >10,000 | Partial, 40% |
| (N88E) | | | | on graph, | |
| | | | | NC ^{a} | |
| 1H3-hIgG1-L6-hIL-2 | 106 | NT | NT | >10,000 | Inactive |
| (N88F) | | | | on graph, | |
| | | | | NC ^{a} | |
| 1H3-hIgG1-L6-hIL-2 | 107 | NT | NT | >10,000 | Inactive |
| (N88I) | | | | on graph, | |
| | | | | NC ^{a} | |
| 1H3-hIgG1-L6-hIL-2 | 109 | NT | NT | 1 | Full |
| (I92Y) | | | | | |
| 1H3-hIgG1-L6-hIL-2 | 110 | NT | NT | 8 | Full |
| (I92S) | | | | | |
| 1H3-hIgG1-L6-hIL-2 | 112 | NT | NT | 31 | Full |
| (I92R) | | | | | |
| 1H3-hIgG1-L6-hIL-2 | 113 | 8-20 | Full | 68-365 ^{a} | Partial, |
| (I92D) | | | | | 70-90% |
| 1H3-hIgG1-L6-hIL-2 | 116 | NT | NT | 5 | Full |
| (E95R) | | | | | |
| 1H3-hIgG1-L6-hIL-2 | 92 | 8 | Full | 167 ^{a} | Partial, 80% |
| (D20T) | | | | | |
| 1H3-hIgG1-L6-hIL-2 | 31 | 21 | Partial, | 117 ^{a} | Partial, 80% |
| (D20A) | | | 80% | | |
| 1H3-hIgG1-L6-hIL-2 | 94 | 52 ^{a} | Partial, | 2159 ^{a} | Partial, 30% |
| (D20Y/H16E) | | | 80% | | |
| 1H3-hIgG1-L6-hIL-2 | 119 | >10,000 ^{a} | Partial, | 30 ^{a} | Partial, 20% |
| (D20Y/H16A) | | | 40% | | |
| 1H3-hIgG1-L6-hIL-2 | 120 | >10,000 ^{a} | Partial, | 343 ^{a} | Partial, 20% |
| (D20Y/H16Y) | | | 40% | | |
| 1H3-hIgG1-L6-hIL-2 | 121 | >10,000 ^{a} | Partial, | 4 ^{a} | Partial, 10% |
| (D20Y/192A) | | | 20% | | |
| 1H3-hIgG1-L6-hIL-2 | 122 | >10,000 ^{a} | Partial, | 12 ^{a} | Partial, 10% |
| (D20Y/I92S) | | | 10% | | |

NT = Not Tested; NC = Not Calculated by GraphPad Prism 7
^{a} = Fold change is an estimate only since a full four parameter logistic curve was not reached

TABLE 11
Fold change from rhIL-2 and agonistic activity on 1H3-hIgG1-L6-hIL-2 fusion proteins from Group 4

| | SEQ ID | | | Fold change | Agonistic |
|---|---|---|---|---|---|
| | NO of | Fold change | Agonistic | from rhIL-2 | Activity |
| | hIL-2 | from rhIL-2 | Activity | (TF1 + | (TF1 + IL − |
| Variants | variant | (NK-92) | (NK-92) | IL − 2Rβ) | 2Rβ) |
| 1H3-hIgG1-L6-hCD25(1-164)-L20-hIL-2 (E15A) | 82 | 184 | Full | 20 | Full |
| 1H3-hIgG1-L6-hCD25(1-164)-L20-hIL-2 (D20I) | 89 | >10,000 ^{a} | Partial, 80% | >10,000 on | Inactive |
| | | | | graph, NC ^{a} | |
| 1H3-hIgG1-L6-hCD25(1-164)-L20-hIL-2 (D20S) | 90 | 2403 | Full | >10,000 on | Inactive |
| | | | | graph, NC ^{a} | |
| 1H3-hIgG1-L6-hCD25(1-164)-L20-hIL-2 (D20H) | 91 | >10,000 ^{a} | Partial, 80% | >10,000 on | Inactive |
| | | | | graph, NC ^{a} | |
| 1H3-hIgG1-L6-hCD25(1-164)-L20-hIL-2 | 93 | >10,000 ^{a} | Partial, 60% | >10,000 on | Inactive |
| (D20W) | | | | graph, NC ^{a} | |
| 1H3-hIgG1-L6-hCD25(1-164)-L20-hIL-2 (D20Y) | 94 | >10,000 ^{a} | Partial, 90% | >10,000 on | Inactive |
| | | | | graph, NC ^{a} | |
| 1H3-hIgG1-L6-hCD25(1-164)-L20-hIL-2 (D20R) | 95 | >10,000 ^{a} | Partial, 80% | >10,000 on | Inactive |
| | | | | graph, NC ^{a} | |
| 1H3-hIgG1-L6-hCD25(1-164)-L20-hIL-2 (D20F) | 96 | >10,000 ^{a} | Partial, 70% | >10,000 on | Inactive |
| | | | | graph, NC ^{a} | |
| 1H3-hIgG1-L6-hCD25(1-164)-L20-hIL-2 (D84K) | 100 | >10,000 | Full | >10,000 on | Inactive |
| | | | | graph, NC ^{a} | |
| 1H3-hIgG1-L6-hCD25(1-164)-L20-hIL-2 (S87A) | 101 | 305 | Full | 44 | Full |
| 1H3-hIgG1-L6-hCD25(1-164)-L20-hIL-2 (N88Y) | 102 | >10,000 ^{a} | Partial, 50% | >10,000 on | Inactive |
| | | | | graph, NC ^{a} | |
| 1H3-hIgG1-L6-hCD25(1-164)-L20-hIL-2 (N88D) | 103 | 393 | Full | >10,000 on | Inactive |
| | | | | graph, NC ^{a} | |
| 1H3-hIgG1-L6-hCD25(1-164)-L20-hIL-2 (N88R) | 104 | 274 | Full | >10,000 on | Inactive |
| | | | | graph, NC ^{a} | |
| 1H3-hIgG1-L6-hCD25(1-164)-L20-hIL-2 (N88E) | 105 | >10,000 ^{a} | Full | >10,000 on | Inactive |
| | | | | graph, NC ^{a} | |
| 1H3-hIgG1-L6-hCD25(1-164)-L20-hIL-2 (N88F) | 106 | >10,000 ^{a} | Partial, 80% | >10,000 on | Inactive |
| | | | | graph, NC ^{a} | |
| 1H3-hIgG1-L6-hCD25(1-164)-L20-hIL-2 (N88I) | 107 | 7780 | Full | >10,000 on | Inactive |
| | | | | graph, NC ^{a} | |
| 1H3-hIgG1-L6-hCD25(1-164)-L20-hIL-2 (192A) | 108 | 26 | Full | 95 | Full |
| 1H3-hIgG1-L6-hCD25(1-164)-L20-hIL-2 (E95A) | 115 | 30 | Full | 16 | Full |
| 1H3-hIgG1-L6-hCD25(1-164)-L20-hIL-2 (E95K) | 117 | 792 | Full | 434 ^{a} | Partial, 60% |

NC = Not Calculated by GraphPad Prism 7
^{a} = Fold change is an estimate only since a full four parameter logistic curve was not reached

TABLE 12
Fold change from rhIL-2 and agonistic activity on 1H3-hIgG1-L6-hIL-2 fusion proteins from Group 5

| | SEQ ID | | | Fold change | Agonistic |
|---|---|---|---|---|---|
| | NO of | Fold change | Agonistic | from rhIL-2 | Activity |
| | hIL-2 | from rhIL-2 | Activity | (TF1 + IL − | (TF1 + |
| Variants | variant | (NK-92) | (NK-92) | 2Rβ) | IL − 2Rβ) |
| 1H3-hIgG1-L6-hIL-2 (F42K/D20A) | 129 | >10,000 ^{a} | Partial, 30% | 219 ^{a} | Partial, 50% |
| 1H3-hIgG1-L6-hIL-2 (F42A/D20A) | 130 | >10,000 ^{a} | Partial, 70% | 96 ^{a} | Partial, 70% |
| 1H3-hIgG1-L6-hIL-2 (R38N/D20A) | 134 | 4497 | Partial, 70% | 121 ^{a} | Partial, 70% |
| 1H3-hIgG1-L6-hIL-2 (R38G/D20A) | 135 | 2811 | Partial, 70% | 139 ^{a} | Partial, 70% |
| 1H3-hIgG1-L6-hIL-2 (R38H/D20A) | 136 | 1752 | Partial, 80% | 107 ^{a} | Partial, 70% |
| 1H3-hIgG1-L6-hIL-2 (R38I/D20A) | 137 | 658 | Partial, 70% | 107 ^{a} | Partial, 60% |
| 1H3-hIgG1-L6-hIL-2 (R38L/D20A) | 138 | 532 | Partial, 80% | 125 ^{a} | Partial, 70% |
| 1H3-hIgG1-L6-hIL-2 (R38M/D20A) | 139 | 786 | Partial, 90% | 85 ^{a} | Partial, 90% |
| 1H3-hIgG1-L6-hIL-2 (R38F/D20A) | 140 | 1072 | Partial, 80% | 124 ^{a} | Partial, 90% |
| 1H3-hIgG1-L6-hIL-2 (R38P/D20A) | 141 | 337 | Partial, 70% | 337 ^{a} | Partial, 80% |
| 1H3-hIgG1-L6-hIL-2 (R38S/D20A) | 142 | 571 | Partial, 70% | 571 ^{a} | Partial, 90% |
| 1H3-hIgG1-L6-hIL-2 (R38V/D20A) | 146 | 765 | Partial, 70% | 765 ^{a} | Partial, 90% |
| 1H3-hIgG1-L6-hIL-2 (R38A/D20A) | 147 | 619 | Partial, 80% | 70 ^{a} | Partial, 70% |
| 1H3-hIgG1-L6-hIL-2 (R38Q/D20A) | 148 | 4700 | Partial, 80% | 91 ^{a} | Partial, 70% |
| 1H3-hIgG1-L6-hIL-2 (D20A/R38E) | 149 | >10,000 ^{a} | Partial, | 409 ^{a} | Partial, 50% |
| | | | 60%-Full | | |
| 1H3-hIgG1-L6-hIL-2 (R38D/D20A) | 150 | >10,000 ^{a} | Partial, 70% | 172 ^{a} | Partial, 70% |
| 1H3-hIgG1-L6-hIL-2 (K43E/D20A) | 151 | 584 | Partial, 90% | 231 ^{a} | Partial, 70% |
| 1H3-hIgG1-L6-hIL-2 (F42R/I92D) | 159 | 801 ^{a} | Partial, 40% | 52 ^{a} | Partial, 80% |
| 1H3-hIgG1-L6-hIL-2 (F42H/I92D) | 160 | 194 | Full | 801 ^{a} | Partial, 80% |
| 1H3-hIgG1-L6-hIL-2 (F42A/I92D) | 161 | 338 ^{a} | Partial, 70% | 194 ^{a} | Full |
| 1H3-hIgG1-L6-hIL-2 (R38D/I92D) | 170 | >10,000 ^{a} | Partial, 80% | 338 ^{a} | Partial, 80% |
| 1H3-hIgG1-L6-hIL-2 (R38E/I92D) | 171 | >10,000 ^{a} | Partial, 70% | 51 ^{a} | Partial, 80% |
| 1H3-hIgG1-L6-hIL-2 (R38Q/I92D) | 172 | 561 | Full | 48 ^{a} | Partial, 90% |
| 1H3-hIgG1-L6-hIL-2 (R38E/D84R) | 175 | >10,000 ^{a} | Partial, 60% | 50 ^{a} | Partial, 80% |
| 1H3-hIgG1-L6-hIL-2 (R38E/D84K) | 176 | >10,000 ^{a} | Partial, 40% | 45 ^{a} | Partial, 80% |
| 1H3-hIgG1-L6-hIL-2 (R38D/E61R/D20A) | 179 | >10,000 on | Partial, 40% | 62 ^{a} | Partial, 70% |
| | | graph, NC ^{a} | | | |
| 1H3-hIgG1-L6-hIL-2 (R38E/E61R/D20A) | 180 | >10,000 ^{a} | Partial, 90% | 181 ^{a} | Partial, 60% |
| 1H3-hIgG1-L6-hIL-2 (R38Q/E61R/D20A) | 181 | >10,000 ^{a} | Partial, 40% | 115 ^{a} | Partial, 80% |
| 1H3-hIgG1-L6-hIL-2 (R38A/E61R/D20A) | 182 | >10,000 ^{a} | Partial, 40% | 130 ^{a} | Partial, 60% |
| 1H3-hIgG1-L6-hIL-2 (D20A/E95A/R38A) | 183 | 149-199 | Partial, | 157 ^{a} | Partial, 70% |
| | | | 70-80% | | |
| 1H3-hIgG1-L6-hIL-2 (D20A/E95A/R38D) | 184 | >10,000 | Partial, | 84-508 ^{a} | Full |
| | | | 60-70% | | |
| 1H3-hIgG1-L6-hIL-2 (D20A/E95A/R38E) | 185 | >10,000 ^{a} | Partial, | 188-427 ^{a} | Partial, 70%-Full |
| | | | 70-90% | | |
| 1H3-hIgG1-L6-hIL-2 (D20A/E95A/R38Q) | 186 | 3725 | Partial, 70% | 124-413 ^{a} | Partial, 80%-Full |
| 1H3-hIgG1-L6-hIL-2 (D20A/E95A/F42R) | 187 | >10,000 ^{a} | Partial, 70% | 87 ^{a} | Partial, 60% |
| 1H3-hIgG1-L6-hIL-2 (D20A/E95A/F42A) | 188 | 3000-5718 ^{a} | Partial, 90% | 45-244 ^{a} | Partial, 70%-Full |
| 1H3-hIgG1-L6-hIL-2 (D20A/E95A/F42D) | 189 | >10,000 ^{a} | Partial, 10% | 1451 ^{a} | Partial, 30% |
| 1H3-hIgG1-L6-hIL-2 (D20A/E95A/F42H) | 190 | 3579 | Partial, 80% | 411 ^{a} | Full |
| 1H3-hIgG1-L6-hIL-2 (D20A/E95A/F42K) | 191 | >10,000 ^{a} | Partial, 50% | 82 ^{a} | Partial, 60% |
| 1H3-hIgG1-L6-hIL-2 (D20A/E95A/K43E) | 193 | 386-553 | Partial, | 46-142 ^{a} | Partial, 50-80% |
| | | | 80-90% | | |
| 1H3-hIgG1-L6-hIL-2 (D20A/E95A/Y45A) | 195 | 62 | Partial, 90% | 300 ^{a} | Partial, 70% |
| 1H3-hIgG1-L6-hIL-2 (D20A/E95A/Y45K) | 196 | 7951 ^{a} | Partial, 70% | 205 ^{a} | Partial, 60% |
| 1H3-hIgG1-L6-hIL-2 (D20A/E95A/Y45R) | 198 | >10,000 ^{a} | Partial, 80% | 293 ^{a} | Partial, 60% |
| 1H3-hIgG1-L6-hIL-2 (D20A/E95A/E61A) | 199 | 367 | Partial, 80% | 195 ^{a} | Partial, 60% |
| 1H3-hIgG1-L6-hIL-2 (D20A/E95A/E62A) | 200 | 3265 ^{a} | Partial, 70% | 230 ^{a} | Partial, 50% |
| 1H3-hIgG1-L6-hIL-2 (D20A/E95A/E62R) | 201 | >10,000 ^{a} | Inactive, 5% | >10,000 on | Partial, 10% |
| | | | | graph, NC ^{a} | |
| 1H3-hIgG1-L6-hIL-2 (D20A/E95A/E62K) | 202 | >10,000 ^{a} | Inactive, 10% | >10,000 on | Partial, 10% |
| | | | | graph, NC ^{a} | |
| 1H3-hIgG1-L6-hIL-2 (D20A/E95A/E62Y) | 203 | >10,000 ^{a} | Partial, 70% | 265 ^{a} | Partial, 40% |
| 1H3-hIgG1-L6-hIL-2 (D20A/E95A/E68Y) | 204 | 131 | Partial, 80% | 61 ^{a} | Partial, 50% |
| 1H3-hIgG1-L6-hIL-2 (D20A/E95A/E68A) | 205 | 45 | Partial, 60% | 620 ^{a} | Partial, 60% |
| 1H3-hIgG1-L6-hIL-2 (D20A/E95A/E68L) | 206 | 187 | Partial, 80% | 172 ^{a} | Partial, 40% |
| 1H3-hIgG1-L6-hIL-2 (D20A/E95A/L72R) | 208 | 1178 | Full | 499 ^{a} | Partial, 70% |
| 1H3-hIgG1-L6-hIL-2 (D20A/E95A/L72D) | 210 | >10,000 ^{a} | Partial, 70% | 456-504 ^{a} | Partial, 60-70% |
| 1H3-hIgG1-L6-hIL-2 (D20A/E95A/L72H) | 211 | 798 | Partial, 70% | 117 ^{a} | Partial, 70% |
| 1H3-hIgG1-L6-hIL-2 | 214 | >10,000 ^{a} | Partial, | 155 185 ^{a} | Partial, 70% |
| (D20A/E95A/F42K/Y45R) | | | 60-70% | | |
| 1H3-hIgG1-L6-hIL-2 | 213 | 840 ^{a} | Partial, 70% | 155 ^{a} | Partial, 70% |
| (F42K/Y45R/D20A/S87A) | | | | | |
| 1H3-hIgG1-L6-hIL-2 (D20A/R38E/C125A) | 215 | >10,000 on | Partial, 50% | 183-584 ^{a} | Partial, 50-70% |
| | | graph, NC ^{a} | | | |
| hIgG1-L6-hIL-2(T3A/D20A/R38E) | 216 | >10,000 ^{a} | Partial, | 77-484 ^{a} | Partial, 60% |
| | | | 60-90% | | |
| 1H3-hIgG1-L6-hIL-2 | 217 | >10,000 ^{a} | Partial, 90% | 218-512 ^{a} | Partial, 50-60% |
| (T3A/D20A/R38E/C125A) | | | | | |
| 1H3-hIgG1-L6-hIL-2 (Δ1-3APT/D20A/R38E) | 218 | 24-69 | Full | 6 | Full |
| 1H3-hIgG1-L6-hIL-2 (Δ1- | 219 | 49-619 | Partial, | 165-619 ^{a} | Partial, 40-50% |
| 3APT/D20A/R38E/C125A) | | | 30-70% | | |
| 1H3-hIgG1-L6-hIL-2 (R38E/Q126D) | 225 | >10,000 on | Partial, 60% | >10,000 on | Partial, 40% |
| | | graph, NC ^{a} | | graph, NC ^{a} | |
| 1H3-hIgG1-L6-hIL-2 (F42K/Y45R/Q126D) | 228 | >10,000 ^{a} | Partial, 60% | 226 ^{a} | Partial, 50% |
| 1H3-hIgG1-L6-hIL-2 (D20A/E95A/Q126D) | 229 | >10,000 on | Inactive | >10,000 on | Inactive |
| | | graph, NC ^{a} | | graph, NC ^{a} | |

NC = Not Calculated by GraphPad Prism 7
^{a} = Fold change is an estimate only since a full four parameter logistic curve was not reached

TABLE 13
Fold change from rhIL-2 and agonistic activity on 1H3-hIgG1-L6-hIL-2 fusion proteins from Group 6

| | SEQ ID | | | | Agonistic |
|---|---|---|---|---|---|
| | NO of | Fold change | Agonistic | Fold change | Activity |
| | hIL-2 | from rhIL-2 | Activity | from rhIL-2 | (TF1 + |
| Variants | variant | (NK-92) | (NK-92) | (TF1 + IL − 2Rβ) | IL − 2Rβ) |
| 1H3-hIgG1-L6-hIL-2 | 247 | >10,000 ^{a} | Partial, 60% | >10,000 on graph, NC ^{a} | Partial, 80% |
| (D20A/F42N) | | | | | |
| 1H3-hIgG1-L6-hIL-2 | 248 | >10,000 ^{a} | Partial, 50% | 16 ^{a} | Partial, 70% |
| (D20A/F42Q) | | | | | |
| 1H3-hIgG1-L6-hIL-2 | 254 | >10,000 ^{a} | Partial, 60% | 13 ^{a} | Partial, 80% |
| (D20A/F42P) | | | | | |
| 1H3-hIgG1-L6-hIL-2 | 255 | >10,000 ^{a} | Partial, 60% | 17 ^{a} | Partial, 40% |
| (D20A/F42S) | | | | | |
| 1H3-hIgG1-L6-hIL-2 | 264 | 4717 | Partial, 80% | 32 ^{a} | Partial, 50% |
| (D20A/Y45E) | | | | | |
| 1H3-hIgG1-L6-hIL-2 | 277 | >10,000 ^{a} | Partial, 50% | 24 ^{a} | Partial, 70% |
| (I92D/F42Q) | | | | | |
| 1H3-hIgG1-L6-hIL-2 | 280 | >10,000 ^{a} | Partial, 30% | 117 ^{a} | Partial, 20% |
| (I92D/F42I) | | | | | |
| 1H3-hIgG1-L6-hIL-2 | 282 | >10,000 ^{a} | Partial, 50% | 58 ^{a} | Partial, 90% |
| (I92D/F42K) | | | | | |
| 1H3-hIgG1-L6-hIL-2 | 286 | >10,000 ^{a} | Partial, 60% | >10,000 on graph, NC ^{a} | Partial, 80% |
| (I92D/F42T) | | | | | |
| 1H3-hIgG1-L6-hIL-2 | 307 | >10,000 ^{a} | Partial, 50% | 25 ^{a} | Partial, 40% |
| (R38E/D20S) | | | | | |
| 1H3-hIgG1-L6-hIL-2 | 308 | >10,000 ^{a} | Partial, 70% | 34 ^{a} | Partial, 30% |
| (F42A/N88R) | | | | | |

NC = Not Calculated by GraphPad Prism
^{a} = Fold change is an estimate only since a full four parameter logistic curve was not reached

### Example 5: Testing for Attenuation for the High-Affinity and Intermediate-Affinity hIL-2 Receptor with Cell-Based Proliferation Assays

The attenuation of IL-2 activity of antibody-attenuated hIL-2 fusion proteins from Groups 1-6 in Example 2 (2D12-mIgG1-D265A-L6-hIL-2, 2D12-hIgG1-L6-hIL-2, and 1H3-hIgG1-L6-hIL-2 fusion proteins) were tested in proliferation assays in both the NK-92 and TF1+IL-2Rβ cell lines as described in Protocol E. The results of the assays are provided in Tables 14-19.

Selected 1H3-hIgG1-L6-hIL-2 fusion proteins with substantial attenuation in the pSTAT5 titration curves from Example 4 were tested in this cell-based proliferation assay. pSTAT5 is a downstream read-out of IL-2 activity and assays require only 10 minutes of stimulation which may be a small snapshot of IL-2 dependent activity. For proliferation assays, cells were incubated with 2D12-mIgG1-D265A-L6-hIL-2, 2D12-hIgG1-L6-hIL-2, 1H3-hIgG1-L6-hIL-2 fusion proteins, or recombinant hIL-2 control for 3-4 days, providing a more physiological relevant read-out of IL-2 dependent activity in vivo. Other 2D12-mIgG1-D265A-L6-hIL-2 and 2D12-hIgG1-L6-hIL-2 fusion proteins that were generated but not tested in a pSTAT5 assay were assayed for IL-2 dependent activity using this proliferation assay.

Similar to cell-based pSTAT5 dose-titration experiments, the calculated EC₅₀ as determined from relative luminescence units (RLU) instead of gMFI and analysis of the results were performed identically to Example 4 once EC₅₀ was calculated. Similar to results identified in Example 4, proliferation curves demonstrated that some substitutions that modulated binding to both the alpha chain and beta chain substantially attenuated IL-2 activity on the high affinity receptor in comparison to single substitutions for binding to the alpha or beta chain only. These selected 1H3-hIgG1-L6-hIL-2 fusion proteins were also tested for proliferation on the TF1+IL-2Rβ cell line and demonstrated that some of these same substitutions substantially attenuated IL-2 activity on the intermediate affinity receptor.

TABLE 14
Fold change from rhIL-2 and agonistic activity on 2D12-mIgG1-D265A-L6-hIL-2 or
2D12-hIgG1-L6-hIL-2 fusion proteins from Group 1 in a cell-based proliferation assay

| | SEQ | | | Fold change | Agonistic |
|---|---|---|---|---|---|
| | ID NO | Fold change | Agonistic | from rhIL-2 | Activity |
| | of hIL-2 | from rhIL-2 | Activity | (TF1 + | (TF1 + |
| Variants | variant | (NK-92) | (NK-92) | IL − 2Rβ) | IL − 2Rβ) |
| 2D12-mIgG1-D265A-L6-hIL-2 | 1 | 2 | Full | 0 | Full |
| (F42K) | | | | | |
| 2D12-mIgG1-D265A-L6-hIL-2 | 60 | 3 | Full | 2 | Full |
| (Y45R) | | | | | |
| 2D12-hIgG1-L6-hIL-2 (V69A) | 2 | 0 | Full | 0 | Full |
| 2D12-hIgG1-L6-hIL-2 (V69E) | 3 | 13 | Partial, 60% | 83 | Full |
| 2D12-hIgG1-L6-hIL-2 (V69H) | 6 | 41 | Partial, 60% | 544 | Full |
| 2D12-hIgG1-L6-hIL-2 (V69K) | 8 | >10,000 on | Inactive | 3033 | Partial, 40% |
| | | graph, NC ^{a} | | | |
| 2D12-hIgG1-L6-hIL-2 (V69L) | 9 | 1 | Full | 1 | Full |
| 2D12-hIgG1-L6-hIL-2 (V69F) | 4 | 0 | Full | 1 | Full |
| 2D12-hIgG1-L6-hIL-2 (V69G) | 5 | 108 | Full | 396 | Full |
| 2D12-hIgG1-L6-hIL-2 (V69I) | 7 | 1 | Full | 1 | Full |
| 2D12-hIgG1-L6-hIL-2 (V69M) | 10 | 2 | Full | 3 | Full |
| 2D12-hIgG1-L6-hIL-2 (V69Q) | 11 | 7 | Full | 16 | Full |
| 2D12-mIgG1-D265A-L6-hIL-2 | 581 | 2392 | Partial, 70% | 2973 | Partial, 50% |
| (V69R) | | | | | |
| 2D12-hIgG1-L6-hIL-2 (V69S) | 12 | 6 | Full | 13 | Full |
| 2D12-hIgG1-L6-hIL-2 (V69T) | 13 | 3 | Full | 3 | Full |
| 2D12-hIgG1-L6-hIL-2 (V69W) | 14 | 0 | Full | 1 | Full |
| 2D12-hIgG1-L6-hIL-2 (V69Y) | 15 | 1 | Full | 1 | Full |
| 2D12-mIgG1-D265A-L6-hIL-2 | 31 | 1 | Full | >10,000 ^{a} | Full |
| (D20A) | | | | | |
| 2D12-mIgG1-D265A-L6-hIL-2 | 32 | 0 | Full | >10,000 ^{a} | Full |
| (D20N) | | | | | |
| 2D12-mIgG1-D265A-L6-hIL-2 | 33 | 4 | Full | >10,000 on | Inactive |
| (D20K) | | | | graph, NC ^{a} | |
| 2D12-mIgG1-D265A-L6-hIL-2 | 34 | 0 | Full | 2289 | Full |
| (N88A) | | | | | |
| 2D12-mIgG1-D265A-L6-hIL-2 | 35 | 0 | Full | 2978 | Full |
| (N88G) | | | | | |
| 2D12-mIgG1-D265A-L6-hIL-2 | 36 | 1-3 | Full | >10,000 on | Inactive |
| (N88H) | | | | graph, NC ^{a} | |
| 2D12-mIgG1-D265A-L6-hIL-2 | 37 | 564-9557 | Partial, 40% | >10,000 on | Inactive |
| (N88K) | | | | graph, NC ^{a} | |
| 2D12-mIgG1-D265A-L6-hIL-2 | 375 | 0-12 | Full | 118 | Full |
| (Q126L) | | | | | |
| 2D12-mIgG1-D265A-L6-hIL-2 | 376 | 0-3 | Full | 40 | Full |
| (Q126E) | | | | | |
| 2D12-mIgG1-D265A-L6-hIL-2 | 16 | 44 | Full | >10,000 on | Inactive |
| (F42K/F44K) | | | | graph, NC ^{a} | |
| 2D12-mIgG1-D265A-L6-hIL-2 | 17 | 1-2 | Full | 0-3 | Full |
| (F42K/Y45R) | | | | | |
| 2D12-mIgG1-D265A-L6-hIL-2 | 18 | 1500 | Partial, 80% | 841 | Partial, 80% |
| (F42K/V69R) | | | | | |
| 2D12-mIgG1-D265A-L6-hIL-2 | 19 | 1 | Full | 3 | Full |
| (Y45R/V69R) | | | | | |
| 2D12-mIgG1-D265A-L6-hIL-2 | 38 | 1 | Full | >10,000 on | Inactive |
| (D20A/D84A) | | | | graph, NC ^{a} | |
| 2D12-mIgG1-D265A-L6-hIL-2 | 39 | 0 | Full | >10,000 on | Partial, 40% |
| (D20A/E15A) | | | | graph, NC ^{a} | |
| 2D12-mIgG1-D265A-L6-hIL-2 | 40 | 0 | Full | >10,000 ^{a} | Full |
| (D20A/E95A) | | | | | |
| 2D12-mIgG1-D265A-L6-hIL-2 | 41 | 6 | Partial, 60% | >10,000 on | Inactive |
| (D20A/N88A) | | | | graph, NC ^{a} | |
| 2D12-mIgG1-D265A-L6-hIL-2 | 42 | 0 | Full | >10,000 ^{a} | Full |
| (D20A/S87A) | | | | | |
| 2D12-mIgG1-D265A-L6-hIL-2 | 43 | 0 | Full | >10,000 on | Inactive |
| (D84A/N88A) | | | | graph, NC ^{a} | |
| 2D12-mIgG1-D265A-L6-hIL-2 | 44 | 0 | Full | 4201 | Partial, 80% |
| (E15A/N88A) | | | | | |
| 2D12-mIgG1-D265A-L6-hIL-2 | 45 | 0 | Full | 1521 | Full |
| (S87A/N88A) | | | | | |
| 2D12-mIgG1-D265A-L6-hIL-2 | 20 | NT | NT | 0 | Full |
| (F42K/F44K/Y45R) | | | | | |
| 2D12-mIgG1-D265A-L6-hIL-2 | 574 | 0-1 | Full | 0-3 | Full |
| (F42A/Y45A/L72G) | | | | | |
| 2D12-mIgG1-D265A-L6-hIL-2 | 21 | 1 | Full | 0 | Full |
| (R38A/F42K/Y45R) | | | | | |
| 2D12-mIgG1-D265A-L6-hIL-2 | 22 | 1-3 | Full | 0 | Full |
| (R38E/F42K/Y45R) | | | | | |
| 2D12-mIgG1-D265A-L6-hIL-2 | 23 | 0 | Full | 3 | Full |
| (K43E/F42K/Y45R) | | | | | |
| 2D12-mIgG1-D265A-L6-hIL-2 | 24 | 0 | Full | 3 | Full |
| (K43T/F42K/Y45R) | | | | | |
| 2D12-mIgG1-D265A-L6-hIL-2 | 25 | 1 | Full | 0-3 | Full |
| (F42K/Y45R/E62A) | | | | | |
| 2D12-mIgG1-D265A-L6-hIL-2 | 26 | 1 | Full | 0-2 | Full |
| (P65R/F42K/Y45R) | | | | | |
| 2D12-mIgG1-D265A-L6-hIL-2 | 27 | 1 | Full | 0-2 | Full |
| (P65S/F42K/Y45R) | | | | | |
| 2D12-mIgG1-D265A-L6-hIL-2 | 28 | 1 | Full | 0-4 | Full |
| (V69A/F42K/Y45R) | | | | | |
| 2D12-mIgG1-D265A-L6-hIL-2 | 29 | 1 | Full | 0-5 | Full |
| (V69D/F42K/Y45R) | | | | | |
| 2D12-mIgG1-D265A-L6-hIL-2 | 30 | 1-4 | Full | 0-6 | Full |
| (V69R/F42K/Y45R) | | | | | |

NT = Not Tested
NC = Not Calculated by GraphPad Prism 7
^{a} = Fold change is an estimate only since a full four parameter logistic curve was not reached

TABLE 15
Fold change from rhIL-2 and agonistic activity on 1H3-hIgG1-L6-hIL-2
fusion proteins from Group 2 in a cell-based proliferation assay

| | SEQ ID NO | Fold change | Agonistic | Fold change | Agonistic |
|---|---|---|---|---|---|
| | of hIL-2 | from rhIL-2 | Activity | from rhIL-2 | Activity |
| Variants | variant | (NK-92) | (NK-92) | (TF1 + IL-2Rβ) | (TF1 + IL-2Rβ) |
| 1H3-hIgG1-L6-hIL-2 (R38A) | 46 | 0 | Full | 1 | Full |
| 1H3-hIgG1-L6-hIL-2 (R38D) | 47 | 3 | Full | 0 | Full |
| 1H3-hIgG1-L6-hIL-2 (R38E) | 48 | 7 | Full | 1 ^{a} | Full |
| 1H3-hIgG1-L6-hIL-2 (F42R) | 50 | 6 | Full | 0 ^{a} | Full |
| 1H3-hIgG1-L6-hIL-2 (F42D) | 52 | 10 | Full | 2 | Full |
| 1H3-hIgG1-L6-hIL-2 (K43A) | 54 | 1 ^{a} | Full | 2 | Full |
| 1H3-hIgG1-L6-hIL-2 (Y45R) | 60 | 4 | Full | 0 ^{a} | Full |
| 1H3-hIgG1-L6-hIL-2 (E61A) | 61 | 0 ^{a} | Full | 1 | Full |
| 1H3-hIgG1-L6-hIL-2 (E61R) | 62 | 1 | Full | 1 | Full |
| 1H3-hIgG1-L6-hIL-2 (E61K) | 63 | 1 | Full | 1 | Full |
| 1H3-hIgG1-L6-hIL-2 (E62A) | 64 | 1 | Full | 1 | Full |
| 1H3-hIgG1-L6-hIL-2 (E62R) | 65 | >10,000 on | Inactive | 209 | Partial, 60% |
| | | graph, NC ^{a} | | | |
| 1H3-hIgG1-L6-hIL-2(E62K) | 66 | >10,000 ^{a} | Partial, | 67-99 | Partial, 60% |
| | | | 40-70% | | |
| 1H3-hIgG1-L6-hIL-2 (E62Y) | 67 | 1 | Full | 1 | Full |
| 1H3-hIgG1-L6-hIL-2 (E68K) | 70 | 0 | Full | 1 | Full |
| 1H3-hIgG1-L6-hIL-2 (E68R) | 71 | 1 | Full | 1 | Full |
| 1H3-hIgG1-L6-hIL-2 (L72R) | 74 | 3 | Full | 3 | Full |
| 1H3-hIgG1-L6-hIL-2 (L72D) | 76 | 2 | Full | 0 | Full |
| 1H3-hIgG1-L6-hIL-2 (L72H) | 77 | 1 | Full | 1 | Full |
| 1H3-hIgG1-L6-hIL-2 (R38D/E61R) | 79 | 36 | Full | 2 | Full |
| 1H3-hIgG1-L6-hIL-2 (R38D/E61R/K43E) | 80 | 27 | Full | 1 | Full |
| 1H3-hIgG1-L6-hIL-2 (T3A/F42A/Y45A/ | 81 | 142 | Full | 1-2 | Partial, |
| L72G/C125A) | | | | | 40%-Full |

NT = Not Tested
NC = Not Calculated by GraphPad Prism 7
^{a} = Fold change is an estimate only since a full four parameter logistic curve was not reached

TABLE 16
Fold change from rhIL-2 and agonistic activity on 1H3-hIgG1-L6-hIL-2
fusion proteins from Group 3 in a cell-based proliferation assay

| | SEQ ID NO | Fold change | Agonistic | Fold change | Agonistic |
|---|---|---|---|---|---|
| | of hIL-2 | from rhIL-2 | Activity | from rhIL-2 | Activity |
| Variants | variant | (NK-92) | (NK-92) | (TF1 + IL-2Rβ) | (TF1 + IL-2Rβ) |
| 1H3-hIgG1-L6-hIL-2 (H16E) | 87 | 0 | Full | 34 | Full |
| 1H3-hIgG1-L6-hIL-2 (L19A) | 88 | NT | NT | 1 | Full |
| 1H3-hIgG1-L6-hIL-2 (D20I) | 89 | NT | NT | 8 ^{a} | Partial, 50% |
| 1H3-hIgG1-L6-hIL-2 (D20S) | 90 | 1 | Full | 201-304 | Partial, |
| | | | | | 80%-Full |
| 1H3-hIgG1-L6-hIL-2 (D20H) | 91 | 239 | Full | 986-6461 ^{a} | Partial, |
| | | | | | 50-70% |
| 1H3-hIgG1-L6-hIL-2 (D20W) | 93 | NT | NT | >10,000 on | Inactive |
| | | | | graph, NC ^{a} | |
| 1H3-hIgG1-L6-hIL-2 (D20Y) | 94 | 880-2097 ^{a} | Full | 383 | Full |
| 1H3-hIgG1-L6-hIL-2 (D20R) | 95 | NT | NT | 262-275 | Full |
| 1H3-hIgG1-L6-hIL-2 (D20F) | 96 | NT | NT | >10,000 on | Inactive |
| | | | | graph, NC ^{a} | |
| 1H3-hIgG1-L6-hIL-2 (D84A) | 98 | NT | NT | 21 | Full |
| 1H3-hIgG1-L6-hIL-2 (D84R) | 99 | 9 | Full | 269-455 | Partial, |
| | | | | | 80-90% |
| 1H3-hIgG1-L6-hIL-2 (D84K) | 100 | 4 | Full | 354-385 | Partial, |
| | | | | | 70%-Full |
| 1H3-hIgG1-L6-hIL-2 (N88Y) | 102 | NT | NT | >10,000 on | Inactive |
| | | | | graph, NC ^{a} | |
| 1H3-hIgG1-L6-hIL-2 (N88D) | 103 | 0 | Full | 115-137 | Partial, |
| | | | | | 70%-Full |
| 1H3-hIgG1-L6-hIL-2 (N88R) | 104 | 1-5 | Partial, | 959 | Partial, 80% |
| | | | 80%-Full | | |
| 1H3-hIgG1-L6-hIL-2 (N88E) | 105 | NT | NT | 1162 | Partial, 50% |
| 1H3-hIgG1-L6-hIL-2 (N88F) | 106 | NT | NT | >10,000 on | Inactive |
| | | | | graph, NC ^{a} | |
| 1H3-hIgG1-L6-hIL-2 (N88I) | 107 | NT | NT | >10,000 on | Inactive |
| | | | | graph, NC ^{a} | |
| 1H3-hIgG1-L6-hIL-2 (192Y) | 109 | NT | NT | 1 | Full |
| 1H3-hIgG1-L6-hIL-2 (192S) | 110 | NT | NT | 10 | Full |
| 1H3-hIgG1-L6-hIL-2 (192R) | 112 | NT | NT | 13 | Full |
| 1H3-hIgG1-L6-hIL-2 (192D) | 113 | 15-20 | Full | 609-1006 | Partial, |
| | | | | | 90%-Full |
| 1H3-hIgG1-L6-hIL-2 (E95R) | 116 | NT | NT | 8 | Full |
| 1H3-hIgG1-L6-hIL-2 (D20T) | 92 | 2 | Full | 124-149 | Full |
| 1H3-hIgG1-L6-hIL-2 (D20Y/H16E) | 118 | 5 | Full | 8076 ^{a} | Partial, 80% |

NT = Not Tested
NC = Not Calculated by GraphPad Prism 7
^{a} = Fold change is an estimate only since a full four parameter logistic curve was not reached

TABLE 17
Fold change from rhIL-2 and agonistic activity on 1H3-hIgG1-L6-hIL-2
fusion proteins from Group 4 in a cell-based proliferation assay

| | SEQ ID NO | Fold change | Agonistic | Fold change | Agonistic |
|---|---|---|---|---|---|
| | of hIL-2 | from rhIL-2 | Activity | from rhIL-2 | Activity |
| Variants | variant | (NK-92) | (NK-92) | (TF1 + IL-2Rβ) | (TF1 + IL-2Rβ) |
| 1H3-hIgG1-L6-hCD25(1-164)-L20-hIL-2 | 82 | 14 | Partial, 90% | 13 | Partial, 70% |
| (E15A) | | | | | |
| 1H3-hIgG1-L6-hCD25(1-164)-L20-hIL-2 | 90 | 127 | Full | 8709 ^{a} | Partial, 50% |
| (D20S) | | | | | |
| 1H3-hIgG1-L6-hCD25(1-164)-L20-hIL-2 | 93 | >10,000 on | Inactive | >10,000 on | Inactive |
| (D20W) | | graph, NC ^{a} | | graph, NC ^{a} | |
| 1H3-hIgG1-L6-hCD25(1-164)-L20-hIL-2 | 94 | >10,000 on | Partial, 50% | >10,000 on | Inactive |
| (D20Y) | | graph, NC ^{a} | | graph, NC ^{a} | |
| 1H3-hIgG1-L6-hCD25(1-164)-L20-hIL-2 | 100 | 783 | Partial, 90% | 2051 ^{a} | Partial, 30% |
| (D84K) | | | | | |
| 1H3-hIgG1-L6-hCD25(1-164)-L20-hIL-2 | 101 | 19 | Partial, 90% | 20 | Partial, 90% |
| (S87A) | | | | | |
| 1H3-hIgG1-L6-hCD25(1-164)-L20-hIL-2 | 103 | 50 | Full | 3284 ^{a} | Partial, 90% |
| (N88D) | | | | | |
| 1H3-hIgG1-L6-hCD25(1-164)-L20-hIL-2 | 104 | 42 | Full | 6864 ^{a} | Partial, 50% |
| (N88R) | | | | | |
| 1H3-hIgG1-L6-hCD25(1-164)-L20-hIL-2 | 117 | 51 | Full | 164 | Partial, 80% |
| (E95K) | | | | | |

NC = Not Calculated by GraphPad Prism 7
^{a} = Fold change is an estimate only since a full four parameter logistic curve was not reached

TABLE 18
Fold change from rhIL-2 and agonistic activity on 1H3-hIgG1-L6-hIL-2
fusion proteins from Group 5 in a cell-based proliferation assay

| | SEQ ID NO | Fold change | Agonistic | Fold change | Agonistic |
|---|---|---|---|---|---|
| | of hIL-2 | from rhIL-2 | Activity | from rhIL-2 | Activity |
| Variants | variant | (NK-92) | (NK-92) | (TF1 + IL-2Rβ) | (TF1 + IL-2Rβ) |
| 1H3-hIgG1-L6-hIL-2 (F42K/D20A) | 129 | >10,000 on | Partial, 20% | 3060 ^{a} | Full |
| | | graph, NC ^{a} | | | |
| 1H3-hIgG1-L6-hIL-2 (F42A/D20A) | 130 | >10,000 on | Full | 2081 ^{a} | Full |
| | | graph, NC ^{a} | | | |
| 1H3-hIgG1-L6-hIL-2 (R38P/D20A) | 141 | 86 | Full | 761 | Full |
| 1H3-hIgG1-L6-hIL-2 (R38S/D20A) | 142 | 140 | Full | 662 | Full |
| 1H3-hIgG1-L6-hIL-2 (R38V/D20A) | 146 | 11 | Full | 843 | Full |
| 1H3-hIgG1-L6-hIL-2 (D20A/R38E) | 149 | 1183-2016 | Partial, | >10,000 ^{a} | Partial, |
| | | | 70%-Full | | 80%-Full |
| 1H3-hIgG1-L6-hIL-2 (R38D/D20A) | 150 | 2262 ^{a} | Full | 680 | Full |
| 1H3-hIgG1-L6-hIL-2 (F42R/I92D) | 159 | >10,000 on | Partial, 30% | 1210 | Full |
| | | graph, NC ^{a} | | | |
| 1H3-hIgG1-L6-hIL-2 (F42H/I92D) | 160 | 288 ^{a} | Full | 242 ^{a} | Full |
| 1H3-hIgG1-L6-hIL-2 (F42A/I92D) | 161 | >10,000 on | Partial, 60% | 2275 | Partial, 80% |
| | | graph, NC ^{a} | | | |
| 1H3-hIgG1-L6-hIL-2 (R38D/I92D) | 170 | 746 ^{a} | Partial, 90% | 172 | Full |
| 1H3-hIgG1-L6-hIL-2 (R38E/I92D) | 171 | 1611 ^{a} | Full | 116 | Full |
| 1H3-hIgG1-L6-hIL-2 (R38E/D84R) | 175 | >10,000 on | Full | 147 | Full |
| | | graph, NC ^{a} | | | |
| 1H3-hIgG1-L6-hIL-2 (R38E/D84K) | 176 | >10,000 on | Partial, 70% | 315 | Full |
| | | graph, NC ^{a} | | | |
| 1H3-hIgG1-L6-hIL-2 (R38D/E61R/D20A) | 179 | >10,000 on | Partial, 50% | 984 | Full |
| | | graph, NC ^{a} | | | |
| 1H3-hIgG1-L6-hIL-2(R38E/E61R/D20A) | 180 | >10,000 on | Full | 417 | Full |
| | | graph, NC ^{a} | | | |
| 1H3-hIgG1-L6-hIL-2(R38Q/E61R/D20A) | 181 | >10,000 on | Partial, 80% | 803 | Full |
| | | graph, NC ^{a} | | | |
| 1H3-hIgG1-L6-hIL-2 (R38A/E61R/D20A) | 182 | >10,000 on | Partial, 60% | 1031 | Full |
| | | graph, NC ^{a} | | | |
| 1H3-hIgG1-L6-hIL-2 (D20A/E95A/R38A) | 183 | 42 | Partial, 70% | 537 | Partial, 80% |
| 1H3-hIgG1-L6-hIL-2 (D20A/E95A/R38D) | 184 | 5315 ^{a} | Partial, 50% | 492 | Partial, 80% |
| 1H3-hIgG1-L6-hIL-2 (D20A/E95A/R38E) | 185 | >10,000 on | Full | 439 | Partial, 90% |
| | | graph, NC ^{a} | | | |
| 1H3-hIgG1-L6-hIL-2 (D20A/E95A/R38Q) | 186 | 572 | Full | 859 | Partial, 80% |
| 1H3-hIgG1-L6-hIL-2 (D20A/E95A/F42R) | 187 | 2096 | Partial, 70% | 356 | Partial, 70% |
| 1H3-hIgG1-L6-hIL-2 (D20A/E95A/F42A) | 188 | 369 | Partial, 70% | 73 | Partial, 90% |
| 1H3-hIgG1-L6-hIL-2 (D20A/E95A/F42D) | 189 | >10,000 on | Inactive | >10,000 on | Inactive |
| | | graph, NC ^{a} | | graph, NC ^{a} | |
| 1H3-hIgG1-L6-hIL-2 (D20A/E95A/F42H) | 190 | 641 | Partial, 90% | 320 | Full |
| 1H3-hIgG1-L6-hIL-2 (D20A/E95A/F42K) | 191 | >10,000 on | Inactive | 272 | Partial, 80% |
| | | graph, NC ^{a} | | | |
| 1H3-hIgG1-L6-hIL-2 (D20A/E95A/K43E) | 193 | 80 | Partial, 90% | 1876 ^{a} | Partial, 70% |
| 1H3-hIgG1-L6-hIL-2 (D20A/E95A/Y45A) | 195 | 25 | Full | 82 | Partial, 90% |
| 1H3-hIgG1-L6-hIL-2 (D20A/E95A/Y45K) | 196 | >10,000 on | Partial, 20% | 383 | Partial, 60% |
| | | graph, NC ^{a} | | | |
| 1H3-hIgG1-L6-hIL-2 (D20A/E95A/Y45R) | 198 | >10,000 on | Inactive | 57 | Partial, 50% |
| | | graph, NC ^{a} | | | |
| 1H3-hIgG1-L6-hIL-2 (D20A/E95A/E61A) | 199 | 306 ^{a} | Partial, 80% | 702 | Partial, 80% |
| 1H3-hIgG1-L6-hIL-2 (D20A/E95A/E62A) | 200 | >10,000 on | Partial, 20% | 661 | Partial, 60% |
| | | graph, NC ^{a} | | | |
| 1H3-hIgG1-L6-hIL-2 (D20A/E95A/E62R) | 201 | >10,000 on | Inactive | >10,000 on | Inactive |
| | | graph, NC ^{a} | | graph, NC ^{a} | |
| 1H3-hIgG1-L6-hIL-2 (D20A/E95A/E62K) | 202 | >10,000 on | Inactive | >10,000 on | Inactive |
| | | graph, NC ^{a} | | graph, NC ^{a} | |
| 1H3-hIgG1-L6-hIL-2 (D20A/E95A/E62Y) | 203 | 721 | Partial, 40% | 982 ^{a} | Partial, 30% |
| 1H3-hIgG1-L6-hIL-2 (D20A/E95A/E68Y) | 204 | 11 | Full | 469 | Full |
| 1H3-hIgG1-L6-hIL-2 (D20A/E95A/E68A) | 205 | 6 | Partial, 50% | 535 | Partial, 80% |
| 1H3-hIgG1-L6-hIL-2 (D20A/E95A/E68L) | 206 | 15 | Full | 972 | Partial, 60% |
| 1H3-hIgG1-L6-hIL-2 (D20A/E95A/L72R) | 208 | 655 ^{a} | Partial, 50% | 316 | Partial, 40% |
| 1H3-hIgG1-L6-hIL-2 (D20A/E95A/L72D) | 210 | 5415 | Inactive | 125 | Partial, 90% |
| 1H3-hIgG1-L6-hIL-2 (D20A/E95A/L72H) | 211 | 583 ^{a} | Partial, 60% | 135 | Partial, 80% |
| 1H3-hIgG1-L6-hIL-2 | 214 | >10,000 on | Inactive | 58-209 | Partial, |
| (D20A/E95A/F42K/Y45R) | | graph, NC ^{a} | | | 40-80% |
| 1H3-hIgG1-L6-hIL-2 | 213 | 123 | Full | 0 | Full |
| (F42K/Y45R/D20A/S87A) | | | | | |
| 1H3-hIgG1-L6-hIL-2 (D20A/R38E/C125A) | 215 | >10,000 ^{a} | Partial, | 2102 ^{a} | Partial, 80% |
| | | | 30-90% | | |
| 1H3-hIgG1-L6-hIL-2 (T3A/D20A/R38E) | 216 | 2338-5870 | Partial, | 353-571 | Full |
| | | | 80%-Full | | |
| 1H3-hIgG1-L6-hIL-2 | 217 | >10,000 ^{a} | Full | 1086 ^{a} | Partial, |
| (T3A/D20A/R38E/C125A) | | | | | 80%-Full |
| 1H3-hIgG1-L6-hIL-2 (Δ1-3APT/D20A/R38E) | 218 | 4-16 | Full | 32 | Partial, 90% |
| 1H3-hIgG1-L6-hIL-2 (Δ1- | 219 | >10,000 ^{a} | Inactive - | 993 ^{a} | Partial, 60% |
| 3APT/D20A/R38E/C125A) | | | Partial, 50% | | |
| 1H3-hIgG1-L6-hIL-2 (F42K/Y45R/Q126D) | 228 | >10,000 ^{a} | Partial, 50% | 597 | Partial, 80% |
| 1H3-hIgG1-L6-hIL-2 (D20A/E95A/Q126D) | 229 | 106 ^{a} | Inactive | >10,000 on | Inactive |
| | | | | graph, NC ^{a} | |

NC = Not Calculated by GraphPad Prism 7
^{a} = Fold change is an estimate only since a full four parameter logistic curve was not reached

TABLE 19
Fold change from rhIL-2 and agonistic activity on 1H3-hIgG1-L6-hIL-2
fusion proteins from Group 6 in a cell-based proliferation assay

| | SEQ ID NO | Fold change | Agonistic | Fold change | Agonistic |
|---|---|---|---|---|---|
| | of hIL-2 | from rhIL-2 | Activity | from rhIL-2 | Activity |
| Variants | variant | (NK-92) | (NK-92) | (TF1 + IL-2Rβ) | (TF1 + IL-2Rβ) |
| 1H3-hIgG1-L6-hIL-2 (D20A/E61R) | 230 | 3950 ^{a} | Partial, 10% | 278 | Partial, 50% |
| 1H3-hIgG1-L6-hIL-2 (D20A/F42N) | 247 | >10,000 ^{a} | Partial, 50% | 662 | Partial, 80% |
| 1H3-hIgG1-L6-hIL-2 (D20A/F42Q) | 248 | >10,000 on | Partial, 30% | 630 | Partial, 80% |
| | | graph, NC ^{a} | | | |
| 1H3-hIgG1-L6-hIL-2 (D20A/F42I) | 251 | 1307 ^{a} | Partial, 20% | 494 | Partial, 60% |
| 1H3-hIgG1-L6-hIL-2 (D20A/F42L) | 252 | 68 | Full | 533 | Partial, 60% |
| 1H3-hIgG1-L6-hIL-2 (D20A/F42M) | 253 | 53 | Full | 370 | Partial, 50% |
| 1H3-hIgG1-L6-hIL-2 (D20A/F42P) | 254 | 9374 ^{a} | Partial, 80% | 702 | Partial, 80% |
| 1H3-hIgG1-L6-hIL-2 (D20A/F42S) | 255 | 1286 | Partial, 70% | 687 | Partial, 70% |
| 1H3-hIgG1-L6-hIL-2 (D20A/F42T) | 256 | 1474 ^{a} | Partial, 10% | 622 | Partial, 80% |
| 1H3-hIgG1-L6-hIL-2 (D20A/F42W) | 257 | 414 ^{a} | Partial, 70% | 400 | Partial, 70% |
| 1H3-hIgG1-L6-hIL-2 (D20A/F42Y) | 258 | 322 | Partial, 70% | 545 | Partial, 70% |
| 1H3-hIgG1-L6-hIL-2 (D20A, F42V) | 259 | 5796 ^{a} | Partial, 30% | 579 | Partial, 80% |
| 1H3-hIgG1-L6-hIL-2 (D20A/Y45A) | 260 | 61 | Partial, 80% | 554 | Partial, 80% |
| 1H3-hIgG1-L6-hIL-2 (D20A/Y45N) | 261 | 31 | Full | 390 | Partial, 70% |
| 1H3-hIgG1-L6-hIL-2 (D20A/Y45D) | 262 | 363 | Partial, 60% | 729 | Partial, 70% |
| 1H3-hIgG1-L6-hIL-2 (D20A/Y45Q) | 263 | 730 ^{a} | Partial, 70% | 348 | Partial, 70% |
| 1H3-hIgG1-L6-hIL-2 (D20A/Y45E) | 264 | 1414 ^{a} | Partial, 60% | 486 | Partial, 70% |
| 1H3-hIgG1-L6-hIL-2 (D20A/Y45G) | 265 | 613 ^{a} | Partial, 80% | 392 | Partial, 60% |
| 1H3-hIgG1-L6-hIL-2 (D20A/Y45H) | 266 | 420 ^{a} | Partial, 70% | 427 | Partial, 60% |
| 1H3-hIgG1-L6-hIL-2 (D20A/Y45I) | 267 | 39 | Partial, 70% | 137 | Partial, 30% |
| 1H3-hIgG1-L6-hIL-2 (D20A/Y45L) | 268 | 11 | Full | 426 | Partial, 70% |
| 1H3-hIgG1-L6-hIL-2 (D20A/Y45M) | 269 | 107 | Full | 449 | Partial, 60% |
| 1H3-hIgG1-L6-hIL-2 (D20A/Y45F) | 270 | 25 | Partial, 90% | 272 | Partial, 50% |
| 1H3-hIgG1-L6-hIL-2 (D20A/Y45P) | 271 | 577 ^{a} | Full | 710 | Full |
| 1H3-hIgG1-L6-hIL-2 (I92D/F42Q) | 277 | 7227 ^{a} | Partial, 30% | 872 | Full |
| 1H3-hIgG1-L6-hIL-2 (I92D/F42I) | 280 | 4587 ^{a} | Partial, 10% | 3644 ^{a} | Full |
| 1H3-hIgG1-L6-hIL-2 (I92D/F42K) | 282 | >10,000 ^{a} | Partial, 20% | 848 ^{a} | Full |
| 1H3-hIgG1-L6-hIL-2 (I92D/F42T) | 286 | >10,000 ^{a} | Partial, 40% | 1068 ^{a} | Full |
| 1H3-hIgG1-L6-hIL-2 (I92D/F42W) | 287 | 405 ^{a} | Full | 3954 ^{a} | Full |
| 1H3-hIgG1-L6-hIL-2 (I92D/F42Y) | 288 | 58 | Full | 106 | Full |
| 1H3-hIgG1-L6-hIL-2 (I92D/F42V) | 289 | 1075 ^{a} | Partial, 60% | 343 | Full |
| 1H3-hIgG1-L6-hIL-2 (I92D/Y45A) | 290 | 15 | Full | 285 | Full |
| 1H3-hIgG1-L6-hIL-2 (I92D/Y45Q) | 293 | 53 | Full | 80 | Full |
| 1H3-hIgG1-L6-hIL-2 (I92D/Y45G) | 295 | 41 | Full | 91 | Full |
| 1H3-hIgG1-L6-hIL-2 (I92D/Y45M) | 299 | 7 | Full | 146 | Full |
| 1H3-hIgG1-L6-hIL-2 (I92D/Y45F) | 300 | 33 | Full | 1650 ^{a} | Full |
| 1H3-hIgG1-L6-hIL-2 (I92D/Y45S) | 302 | 20 | Full | 306 | Full |
| 1H3-hIgG1-L6-hIL-2 (R38E/D20H) | 306 | 13 | Inactive | 2945 | Partial, 40% |
| 1H3-hIgG1-L6-hIL-2 (R38E/D20S) | 307 | >10,000 on | Partial, 50% | 626 | Full |
| | | graph, NC ^{a} | | | |
| 1H3-hIgG1-L6-hIL-2 (F42A/N88R) | 308 | 8351 ^{a} | Partial, 70% | 1456 | Full |
| 1H3-hIgG1-L6-hIL-2 (F42A/N88D) | 309 | 1966 ^{a} | Full | 86 | Full |
| 1H3-hIgG1-L6-hIL-2 (R38E/I92K) | 334 | >10,000 ^{a} | Partial, 70% | 239 | Full |
| 1H3-hIgG1-L6-hIL-2 (R38E/I92P) | 337 | >10,000 on | Inactive | >10,000 on | Inactive |
| | | graph, NC ^{a} | | graph, NC ^{a} | |
| 1H3-hIgG1-L6-hIL-2 (R38E/H16E) | 343 | 658 ^{a} | Full | 58 | Full |
| 1H3-hIgG1-L6-hIL-2 (R38K/D20A) | 344 | 66 | Full | 369 | Partial, 70% |

NC = Not Calculated by GraphPad Prism 7
^{a} = Fold change is an estimate only since a full four parameter logistic curve was not reached

### Example 6: Generation of Anti-hPD-1 Antibodies

Several approaches were used to generate a variety of different anti-hPD-1 antibodies with desired properties.

In one approach, anti-hPD-1 human monoclonal antibodies were generated using transgenic chickens (OmniChicken™) that express human antibody genes (human light chain (VLCL or VKCK) and human VH) and the chicken constant regions of the heavy chain (Ching et al., mAbs 2018). Transgenic chickens were immunized with 100 μg of Fc-tagged human PD-1 protein (huPD-1-Fc) (SEQ ID NO: 380) every 14 days for 14 weeks. In another approach, transgenic chickens were genetically immunized six times with DNA encoding human PD-1 (SEQ ID NO: 347) followed by a final boost with 100 μg huPD-1-Fc (SEQ ID NO: 380). The serum immune response of each animal was monitored by ELISA against biotinylated human PD-1 on streptavidin coated plates.

Splenocytes were isolated from each immunized animal, tested for positive antibody clones using the Gel Encapsulated Microenvironment (GEM) assay (as described in Mettler Izquierdo, S., Varela, S., Park, M., Collarini, E. J., Lu, D., Pramanick, S., Rucker, J., Lopalco, L., Etches, R., & Harriman, W. (2016). High-efficiency antibody discovery achieved with multiplexed microscopy. Microscopy (Oxford, England), 65 (4), 341-352) and screened against human PD-1 labelled beads. Positive clones were sequenced and variable regions of the heavy and light chains were cloned, assembled into a single chain variable fragment, and fused to the hinge and Fc regions of immunoglobulins (ScFv-Fc). These unique scFv-Fc fusion proteins were transiently expressed in Expi293 cells and supernatants were tested for binding activity by ELISA on plates coated with huPD-1-Fc (SEQ ID NO: 380) or cynomolgous-PD-1-Fc (SEQ ID NO: 381). In total, 102 unique anti-human PD-1 variable heavy and variable light pairings were identified using this method. 2H7-hIgG4 (SEQ ID NOs: 382-391, 424, and 425) and A2-hIgG4 (SEQ ID NOs: 402-411, 428, and 429) were among the antibodies identified in this approach.

Other approaches led to the identification of an anti-hPD-1 antibody denoted as C51E6-hIgG4, which was germline optimized to become the antibody designated C51E6-5-hIgG4 (SEQ ID NOs: 392-401, 426, 427), and humanized and further sequence optimized to become the antibody designated Abz1mod-hIgG4 (SEQ ID NOs: 449, 450).

The anti-PD-1 variable region sequences were expressed as human IgG4 kappa antibodies and were evaluated for the ability to bind to PD-1 expressing cells using flow cytometry as described in General Methods Protocol A. Antibodies to be tested were first screened for binding to human PD-1 using a Jurkat cell line expressing recombinant human PD-1 (Jurkat+hPD-1 cell line). Antibodies were serially diluted from a top concentration of 280 nM and Allophycocyanin-conjugated anti-human IgG secondary antibody was then added to cells for detection. Of 92 hits, 79 test anti-PD-1 antibodies had an EC₅₀ binding (by flow cytometry) of <30 nM. 2H7-hIgG4 (SEQ ID NOs: 382-391, 424, and 425), C51E6-5-hIgG4 (SEQ ID NOs: 392-401, 426, and 427), A2-hIgG4 (SEQ ID NOs: 402-411, 428, and 429), OMC.1.B6-hIgG4 (SEQ ID NOs: 438 and 439), OMC.1.D6-hIgG4 (SEQ ID NOs: 442 and 443), OMC.2.C6-hIgG4 (SEQ ID NOs: 440 and 441), 1H9-hIgG4 (SEQ ID NOs: 576 and 525), 1D5-hIgG4 (SEQ ID NOs: 577 and 527), and 2A3.H7-hIgG4 (SEQ ID NOs: 424 and 523) were among a group of antibodies identified as antibodies with medium to high affinity binding to hPD-1 using a Jurkat cell line expressing human PD-1 (SEQ ID NO: 346). The calculated EC₅₀ of binding to Jurkat cells which recombinantly expressed hPD-1 by flow cytometry in multiple experiments was 0.1-0.3 nM for 2H7-hIgG4, 1H9-hIgG4, 1D5-hIgG4, and 2A3.H7-hIgG4. The calculated EC₅₀ of binding to Jurkat cells expressing hPD-1 by flow for C51E6-5-hIgG4 was 2-4 nM, and 3-16 nM for A2-hIgG4, OMC.1.B6-hIgG4, OMC.1.D6-hIgG4, and OMC.2.C6-hIgG4. Binding was specific to hPD-1 since 2H7-hIgG4, C51E6-5-hIgG4, A2-hIgG4, 1H9-hIgG4, 1D5-hIgG4, 2A3.H7-hIgG4, OMC.1.B6-hIgG4, OMC.1.D6-hIgG4, and OMC.2.C6-hIgG4 antibody titrations did not bind the parental Jurkat cell line which did not express hPD-1 (data not shown).

### Example 7: Characterization of Anti-hPD-1 Antibody Binding in the Presence of Anti-hPD-1 #1-mIgG2b-N297A and anti-hPD-1 #2-mIgG2b-N297A antibodies

2H7-hIgG4, C51E6-5-hIgG4, and A2-hIgG4 were assessed for binding competition to hPD-1 in the presence of anti-hPD-1 #1-mIgG2b-N297A and anti-hPD-1 #2-mIgG2b-N297A as described in General Methods Protocol B.

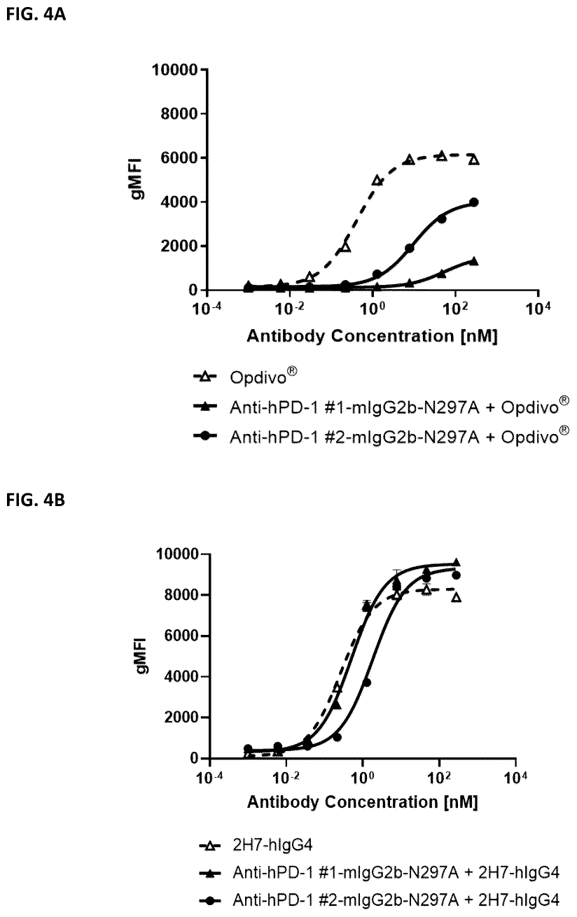

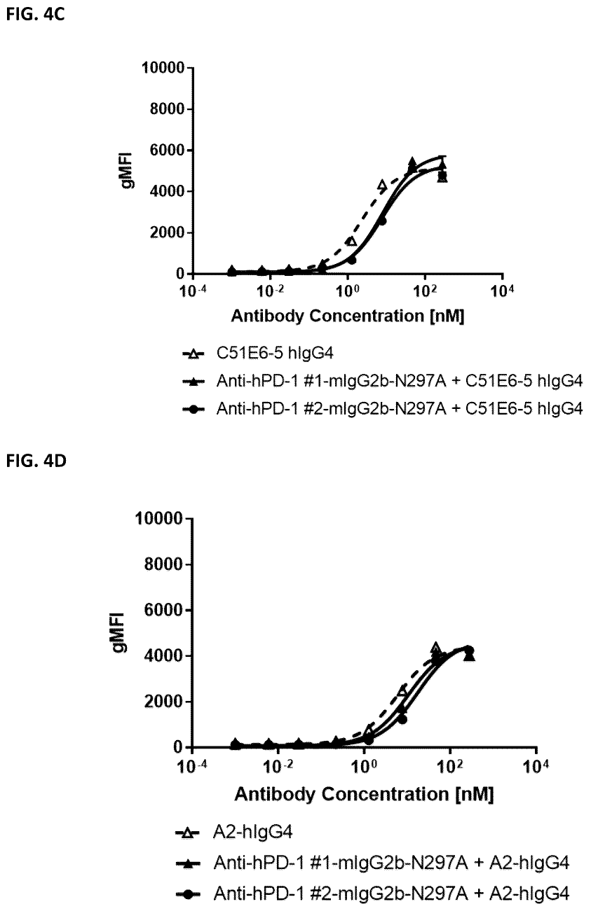

As a control, OPDIVO® (nivolumab) was titrated in the presence of saturating concentrations of 10 μM anti-hPD-1 #1-mIgG2b-N297A (FIG. 4A). The dose-titration curve in the presence of anti-hPD-1 #1-mIgG2b-N297A competitor was greatly reduced (100 to 1000-fold shift of the dose-titration curve to the right of the graph) when compared to the dose-titration curve of OPDIVO® without anti-hPD-1 #1-mIgG2b-N297A competitor. The addition of anti-hPD-1 #1-mIgG2b-N297A or anti-hPD-1 #2-mIgG2b-N297A at saturating concentrations (10 μM) prior to exposure with 2H7-hIgG4, C51E6-5-hIgG4, or A2-hIgG4 did not abrogate binding of 2H7-hIgG4, C51E6-5-hIgG4, or A2-hIgG4 to hPD-1 as illustrated by less than 10-fold shift in FIG. 4B-4D, suggesting that 2H7-hIgG4, C51E6-5-hIgG4 and A2-hIgG4 did not compete for binding to PD-1 in the presence of anti-hPD-1 #1-mIgG2b-N297A or anti-hPD-1 #2-mIgG2b-N297A.

### Example 8: Characterization of Non-Antagonist hPD-1 Antibodies

Anti-hPD-1 antibodies 2H7-hIgG4, C51E6-5-hIgG4 and A2-hIgG4 were tested for PD-1 antagonist activity using an in vitro cell-based human PD-1/PD-L1 blockade bioassay as described in General Methods Protocol C. All antibodies except A2-hIgG4 were tested at 200 nM final concentration. A2-hIgG4 was tested at 500 nM final concentration.

None of the anti-hPD-1 antibodies 2H7-hIgG4, C51E6-5-hIgG4, A2-hIgG4, OMC.1.B6-hIgG4, OMC.1.D6-hIgG4, OMC.2.C6-hIgG4, 1H9-hIgG4, 1D5-hIgG4, and 2A3.H7-hIgG4 demonstrated hPD-1 antagonist activity, as all displayed luminescence levels of an average of 3000 relative luminescence units (RLU) and exhibited an RLU similar to the negative control KLH-C3-hIgG4 (data not shown). In contrast, the anti-hPD-1 #1, which is a known hPD-1 antagonist that blocks hPD-L1 (SEQ ID NO: 584) engagement with hPD-1, exhibited luminescence of above 14,000 RLU (data not shown).

### Example 9: Anti-hPD-1-Attenuated hIL-2 Fusion Proteins Bind Jurkat Cells Expressing Human PD-1

In order to construct various antibody and antibody-attenuated hIL-2 fusion protein expression vectors, the corresponding polynucleotide encoding sequences of antibody, cytokines, cytokine receptors and linkers were generated and cloned into expression vectors. The antibodies or antibody fusion proteins were transiently expressed in Human Embryonic Kidney (HEK) 293 cells, then purified by affinity chromatography using Protein A- or Protein G-Sepharose. The purified proteins were concentrated and buffer-exchanged to phosphate buffered saline or phosphate buffered saline containing 100 mM L-arginine and 10 mM L-histidine using ultracentrifugal filtration, after which protein concentration was determined.

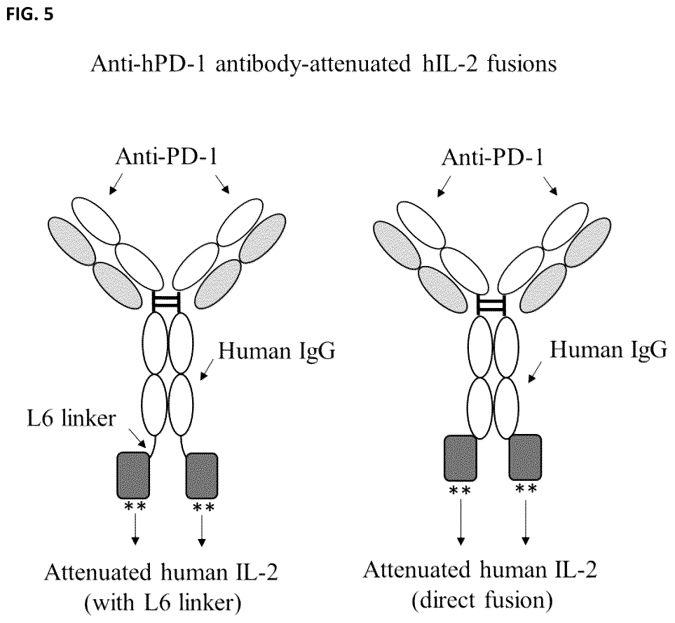

In some approaches, 2H7-hIgG4, C51E6-5-hIgG4, and A2-hIgG4 carrying an S228P hinge stabilization mutation were directly fused (df) to hIL-2 or fused to hIL-2 at the C-terminus of the immunoglobulin heavy chain using the L6 linker. An illustration of these anti-PD-1-attenuated hIL-2 fusion proteins is summarized in FIG. 5. Various constructs were generated with the substitutions in hIL-2 that attenuated hIL-2 activity as described in Example 2. Anti-hPD-1-attenuated hIL-2 fusion proteins listed in Table 20 were tested for binding to hPD-1 using the Jurkat cell line expressing hPD-1 as described in General Methods Protocol A. The variable region of 2H7-hIgG4 (SEQ ID NOs: 384 and 385) was further optimized, and the isotype was switched to a human IgG1 with the effector function null substitutions L235A/G237A (LAGA, as described in WO1998/006248) to become H7-632-hIgG1-LAGA (SEQ ID NOs: 414 and 415). The optimized H7-632-hIgG1-LAGA was also directly fused (df) to a variant of hIL-2 with attenuated hIL-2 activity (hIL-2 T3A/D20A/R38E/C125A; SEQ ID NO: 217) to become H7-767 (SEQ ID NOs: 412-413, 415-423, 532) and both H7-632-hIgG1-LAGA and H7-767 were tested for binding to hPD-1 (Table 20). EC₅₀ values were calculated from the geometric mean fluorescent intensity (gMFI) across the titrated concentrations using GraphPad Prism 7 software.

The generation of anti-hPD-1-attenuated hIL-2 fusion proteins did not reduce binding to hPD-1, and the anti-hPD-1-attenuated hIL-2 fusion proteins were still able to bind to Jurkat cells expressing human PD-1. The calculated EC₅₀ of tested anti-hPD-1-attenuated hIL-2 fusion proteins in comparison to respective anti-hPD-1 antibody without the attenuated hIL-2 moiety is summarized in Table 20.

TABLE 20
Anti-hPD-1-attenuated hIL-2 fusion protein binding
(EC₅₀) to hPD-1 expressing Jurkat cell line by flow cytometry

| | EC₅₀ | Corresponding Anti-hPD-1-hIL-2 | EC₅₀ |
|---|---|---|---|
| Anti-hPD-1 Antibody | (nM) | Fusion Protein | (nM) |
| Anti-hPD-1 #1 | 0.3586 | Anti-hPD-1 #1-hIgG4-L6-hIL-2 (D20A/R38E) | 0.5318 |
| C51E6-5-hIgG4 | 2.048 | C51E6-5-hIgG4-L6-hIL-2 (D20A/R38E) | 1.587 |
| OMC.1.B6-hIgG4 | 7.422 | OMC.1.B6-hIgG4-L6-hIL-2 (D20A/R38E) | 5.635 |
| OMC.2.C6-hIgG4 | 11.52 | OMC.2.C6-hIgG4-L6-hIL-2 (D20A/R38E) | 16.32 |
| OMC.1.D6-hIgG4 | 15.96 | OMC.1.D6-hIgG4-L6-hIL-2 (D20A/R38E) | 8.87 |
| A2-hIgG4 | 5.968 | A2-hIgG4-df-hIL-2 (D20A/R38E) | 10.67 |
| D12-hIgG4 | 9.674 | D12-hIgG4-df-hIL-2 (D20A/R38E) | 17.09 |
| G12-hIgG4 | 5.36 | G12-hIgG4-df-hIL-2 (D20A/R38E) | 7.578 |
| 2H7-hIgG4 | 0.2769 | 2H7-hIgG4-df-hIL-2 (D20A/R38E) | 0.1946 |
| H7-632-hIgG1-LAGA | 0.115 | H7-767 | 0.218 |

The addition of the attenuated hIL-2 moiety on anti-hPD-1 antibodies did not abrogate binding to human PD-1 as demonstrated by a less than 2-fold increase in EC₅₀ binding of anti-hPD-1-hIL-2 fusion proteins to Jurkat+hPD-1 cells in comparison to the anti-hPD-1 antibody without the attenuated hIL-2 moiety.

### Example 10: Anti-hPD-1-Attenuated hIL-2 Fusion Proteins Bind hPD-1 in the Presence of Anti-hPD-1 #1 and Anti-hPD-1 #2 Antibodies

Anti-hPD-1-attenuated hIL-2 fusion proteins were tested for binding to the hPD-1 receptor in the presence of anti-hPD-1 #1 and anti-hPD-1 #2 as described in General Methods Protocol B and Example 7. The converse experiment was also performed in which anti-hPD-1 #1-mIgG2b-N297A or anti-hPD-1 #2-mIgG2b-N297A was examined for binding to hPD-1 in the presence of saturating concentrations of test antibody-attenuated hIL-2 fusion proteins. In this format, Jurkat cells expressing hPD-1 were plated at 100,000 cells per well in FACS buffer, blocked with anti-human FcγR Blocking Reagent (Miltenyi) for 10 minutes at 4° C. and washed. Test antibody-attenuated hIL-2 fusion proteins 2H7-hIgG4-df-hIL-2 (D20A/R38E), C51E6-5-hIgG4-L6-hIL-2 (D20A/R38E), A2-hIgG4-df-hIL-2 (D20A/R38E), H7-767, and isotype control anti-DNase 1H3-hIgG4-df-hIL-2 (D20A/R38E) were diluted to 280 nM final concentration in 100 μL FACS buffer and incubated with Jurkat cells expressing hPD-1 cells for 1 hour on ice. Cells were washed and re-suspended in FACS buffer containing six-fold serial titrations of anti-hPD-1 #1-mIgG2b-N297A or anti-hPD-1 #2-mIgG2b-N297A starting at a maximum concentration of 50 nM for 1 hour on ice. Cells were washed and re-suspended in 1:100 dilution of Phycoerythrin-conjugated anti-mouse IgG light chain kappa monoclonal antibody for 45 minutes on ice. Cells were again washed and re-suspended in FACS buffer with 1:1000 dilution of Sytox Green (Thermo Fisher). Flow cytometry analysis was performed using the BD FACS Canto II (BD Biosciences) and gMFI calculated using FlowJo software version 10. EC₅₀ values were calculated from the gMFI of the Phycoerythrin signal across the titrated concentrations using GraphPad Prism 7 software.

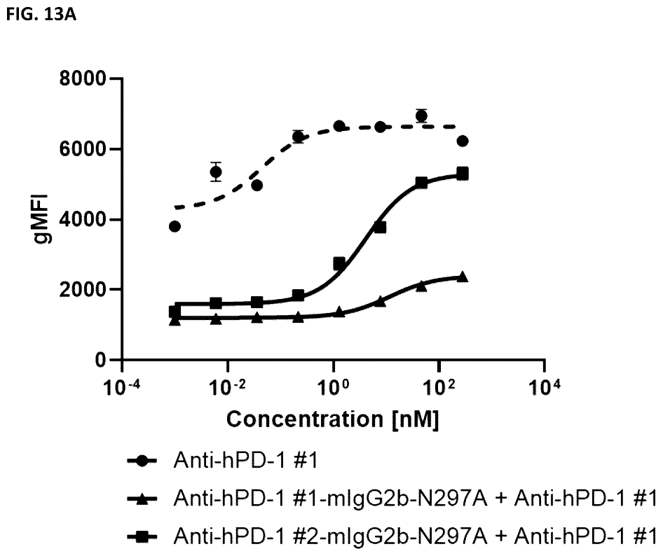

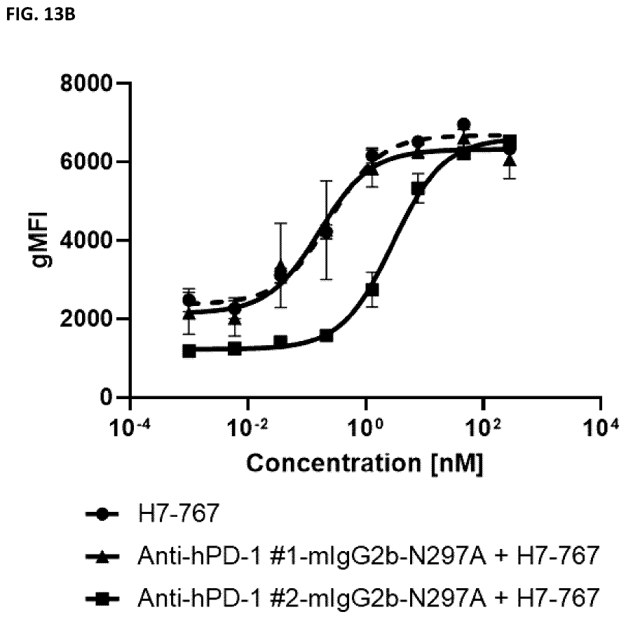

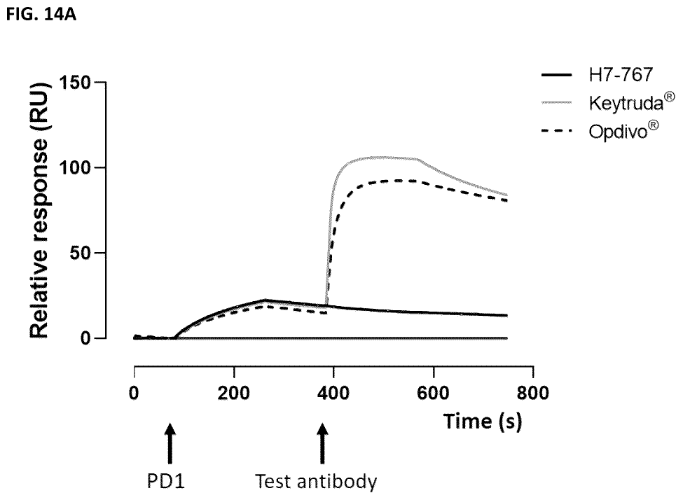

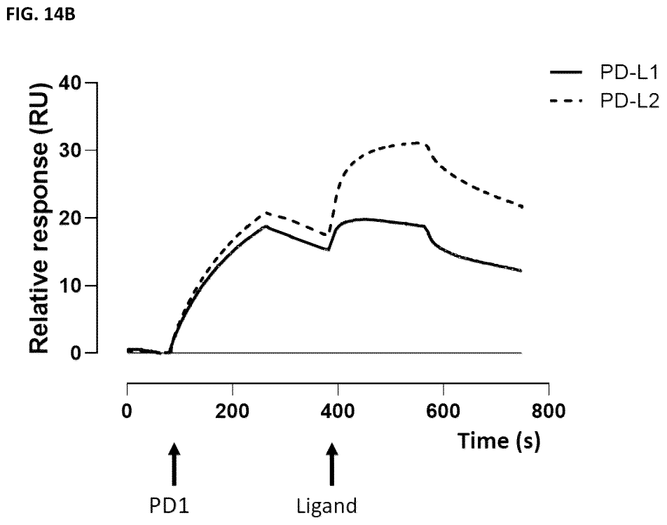

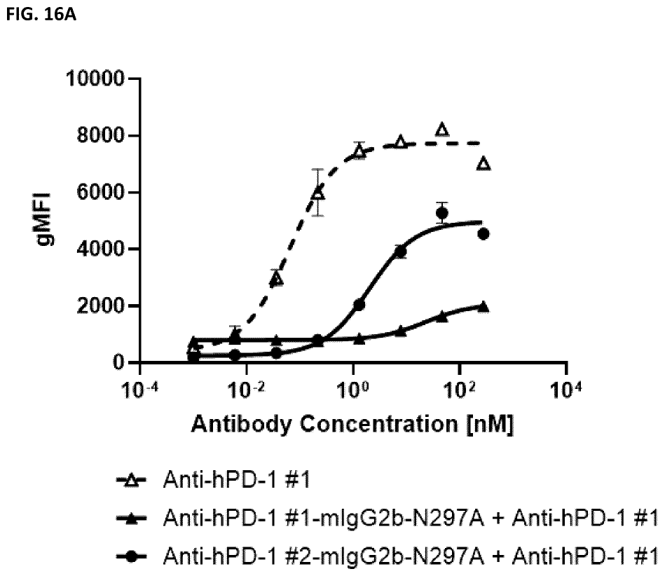

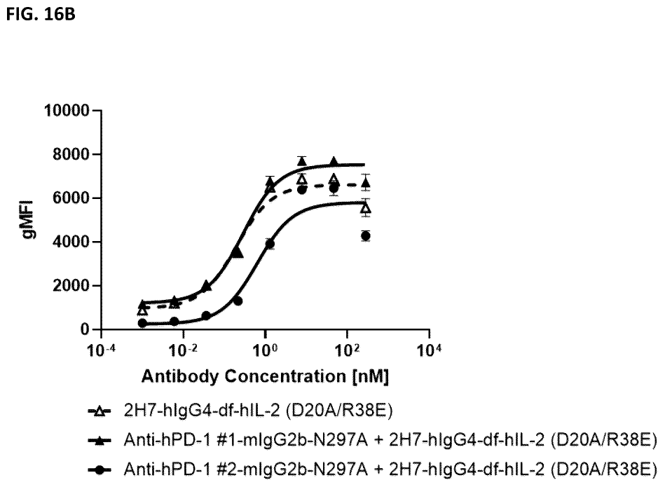

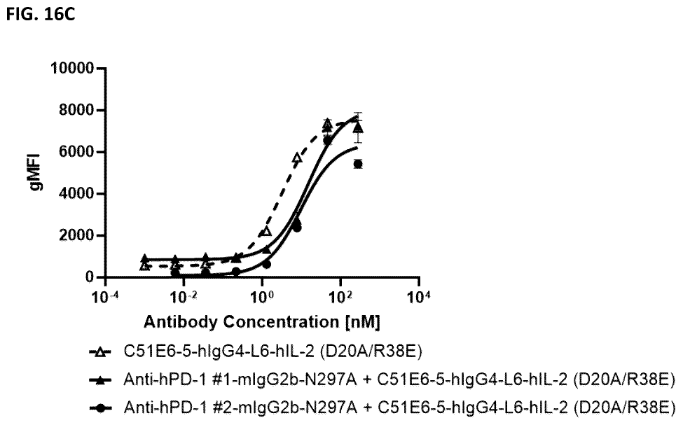

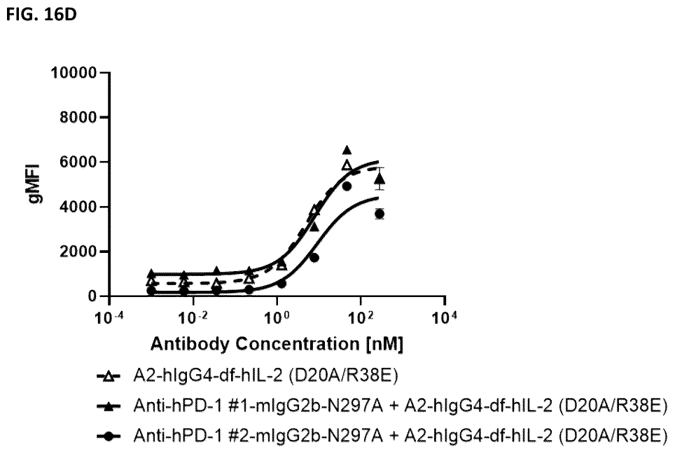

The addition of the attenuated hIL-2 to anti-hPD-1 antibodies 2H7-hIgG4, C51E6-5-hIgG4, and A2-hIgG4 did not diminish the ability of the anti-hPD-1 proteins to bind to human PD-1 in the presence of anti-hPD-1 #1-mIgG2b-N297A or anti-hPD-1 #2-mIgG2b-N297A, similar to the results described in Example 7 (FIG. 16B-16D). H7-767 was also tested in this competition assay and FIG. 13B illustrates that H7-767 continues to bind to the hPD-1 receptor in the presence of anti-hPD-1-#1-mIgG2b-N297A or anti-hPD-1 #2-mIgG2b-N297A. In contrast, the binding of the positive control anti-hPD-1 #1 was substantially decreased in the presence of anti-hPD-1 #1-mIgG2b-N297A or anti-hPD-1 #2-mIgG2b-N297A (FIG. 16A, 13A).

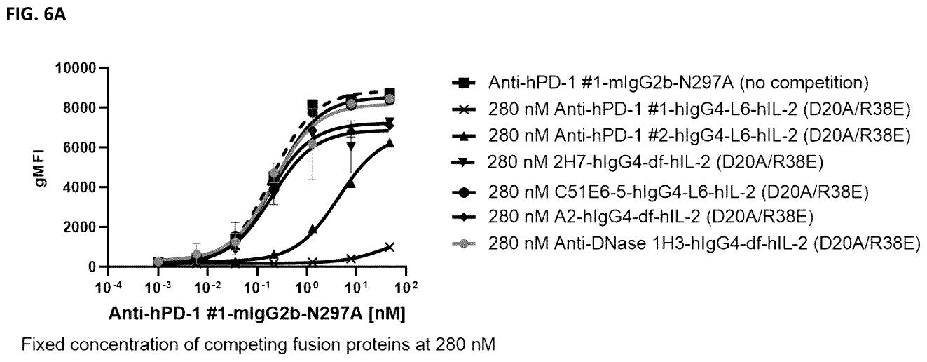

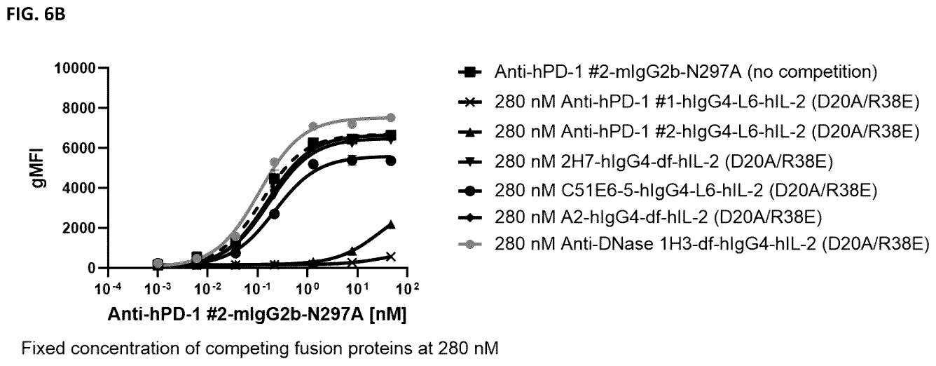

FIG. 6A and FIG. 6B show that for the converse competition assay, anti-hPD-1 #1-mIgG2b-N297A (FIG. 6A) and anti-hPD-1 #2-mIgG2b-N297A (FIG. 6B) were still able to bind to hPD-1 on Jurkat cells in the presence of saturating (280 nM) anti-hPD-1-attenuated hIL-2 fusion proteins 2H7-hIgG4-df-hIL-2 (D20A/R38E), C51E6-5-hIgG4-df-hIL-2 (D20A/R38E) or A2-hIgG4-df-hIL-2 (D20A/R38E). These binding curves with saturating anti-hPD-1-attenuated hIL-2 fusion proteins prior to exposure with anti-hPD-1 fusion proteins overlapped with binding curves of anti-hPD-1 #1-mIgG2b-N297A (no competition) or anti-hPD-1 #2-mIgG2b-N297A (no competition). The binding curves also overlapped with a saturating negative control fusion protein, 1H3-hIgG4-df-hIL-2 (D20A/R38E) that did not bind to hPD-1.

### Example 11: Anti-hPD-1-Attenuated hIL-2 Fusion Proteins Bind Recombinantly-Expressed Cynomolgus PD-1

Anti-hPD-1-attenuated hIL-2 fusion proteins were tested in flow cytometry for binding to cynomolgus PD-1 using a human Embryonic Kidney 293 cell line expressing the SV40 large T cell antigen (HEK-293T) that was transiently transfected to recombinantly express cynomolgus PD-1. For each transfection reaction, 2 million HEK-293T cells were transfected with 2 μg of pCMV6-hygro-HA-cyno-PD-1 (1-185) (SEQ ID NO: 448), a mammalian vector comprising the cynomolgus PD-1 extracellular domain tagged with a human influenza hemagglutinin and the sequence encoding for hygromycin resistance. Transfection was performed by electroporation. Transfected cells were blocked with human FcγR blocking reagent and stained with titrating amounts of anti-hPD-1-attenuated hIL-2 fusion proteins. Additionally, Phycoerythrin conjugated anti-hemagglutinin clone 15B12 was added to cells to stain for transfected cells and Allophycocyanin-conjugated anti-human IgG Fc secondary clone HP6017 (BioLegend Cat #409306) was added to cells to stain bound antibody. The cells were analyzed on the BD Canto II and FlowJo software version 10 was used to gate on live, transfected (hemagglutinin-positive) cells and to calculate gMFI of the Allophycocyanin signal. EC₅₀ values were calculated from the gMFI across the titrated concentrations using GraphPad Prism 7 software.

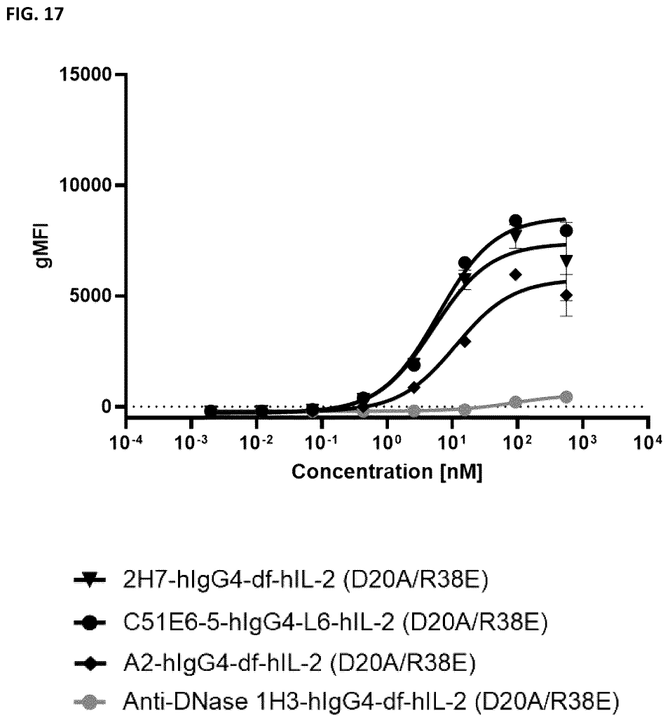

Anti-hPD-1-attenuated hIL-2 fusion proteins bound to cynomolgus PD-1-expressing HEK-293T cells in a similar fashion to the binding profile seen on Jurkat T cells expressing human PD-1 (FIG. 17). The EC₅₀ for binding to cynomolgus PD-1 expressing HEK-293T cells was 5 nM for 2H7-hIgG4-df-hIL-2 (D20A/R38E), 6 nM for C51E6-5-hIgG4-df-hIL-2 (D20A/R38E), and 11 nM for A2-hIgG4-df-hIL-2 (D20A/R38E). Anti-hPD-1 #1 and anti-hPD-1 #2 which were formatted as comparator anti-hPD-1-attenuated hIL-2 fusion proteins also bound to cynomolgus PD-1 with EC₅₀ values of 9 nM and 2 nM, respectively, suggesting that the addition of the attenuated hIL-2 moiety on the anti-hPD-1 antibodies did not abrogate binding to cynomolgus PD-1.

### Example 12: Anti-hPD-1-Attenuated hIL-2 Fusion Proteins Bind Activated Primary Human and Cynomolgus PD-1

The binding of anti-hPD-1 antibodies and anti-hPD-1-attenuated hIL-2 fusion proteins on activated primary T cells expressing hPD-1 was examined by flow cytometry. To test if 2H7-hIgG4, C51E6-5-hIgG4, or A2-hIgG4 bound to native hPD-1, cryopreserved human peripheral blood mononuclear cells (PBMCs) were thawed and activated with 50 ng/ml phorbol 12-myristate 13-acetate (PMA) and 1 μg/mL ionomycin to up-regulate the hPD-1 receptor. Activated PBMCs were collected, blocked with 1:50 dilution of Human FcγR Blocking Reagent (Miltenyi) for 10 minutes at 4° C., and stained with titrated concentrations of anti-hPD-1 antibodies 2H7-hIgG4, C51E6-5-hIgG4, A2-hIgG4, anti-hPD-1 #1, and isotype control. Cells were then stained with 1:20 dilution of Allophycocyanin-conjugated anti-human IgG Fc to detect bound antibody. To delineate immune subsets, a cocktail of surface markers included anti-human CD3, anti-CD4, and anti-CD8 antibodies was used. In addition, a sample fraction was examined for cellular expression of hPD-1, hCD25, hCD122, and hCD132. Cells were analyzed on the BD Fortessa (BD Biosciences), FlowJo software version 10 was used to gate on T cell subsets then calculate gMFI of the allophycocyanin signal. EC₅₀ values were calculated from the gMFI across the titrated concentrations using GraphPad Prism 7 software. To test the binding of anti-hPD-1-hIL-2 fusion proteins, cryopreserved CD3+ T cells were activated with PMA/ionomycin and flow cytometry binding was performed identically as described above.

Human PD-1 antibody-attenuated hIL-2 fusion proteins were also tested for binding to activated cynomolgus T cells using flow cytometry. Cynomolgus PBMCs were activated with a mixture of 0.081 μM PMA and 1.34 μM ionomycin. 24 hours later, cells were stained using the same procedure as binding to human PD-1 primary cells described above except cynomolgus cross-reactive markers were used. FlowJo software version 10 was used to gate on live, CD3⁺CD4⁺ or CD3⁺CD8⁺ T cells and then to calculate gMFI of the Allophycocyanin signal. EC₅₀ values were calculated from the gMFI across the titrated concentrations of anti-hPD-1 antibodies or hPD-1 antibody-attenuated hIL-2 fusion proteins using GraphPad Prism 7 software.

In some variants tested, the attenuated hIL-2 also included the substitutions T3A and C125A, which remove a site for O-linked glycosylation and substitute away a free cysteine residue, respectively.

40-50% of CD4 T cells were PD-1⁺ while 30-40% of CD8⁺ T cells were PD-1⁺ after PMA and ionomycin activation (data not shown). The calculated EC₅₀ for binding to activated human CD3⁺CD4⁺ T cells by flow cytometry was 0.1-0.7 nM for 2H7-hIgG4, 12 nM for C51E6-5-hIgG4, 30 nM for A2-hIgG4, and 0.04 nM for 2H7-hIgG4-df-hIL-2 (T3A/D20A/R38E/C125A). The EC₅₀ for binding to activated human CD3⁺CD8+ T cells was 0.1-0.8 nM for 2H7-hIgG4, 16 nM for C51E6-5-hIgG4, 22 nM for A2-hIgG4, and 0.03 nM for 2H7-hIgG4-df-hIL-2 (T3A/D20A/R38E/C125A). The EC₅₀ for binding to activated human CD3⁺CD4⁺ T cells was 0.19 nM and activated human CD3 CD8⁺ T cells was 0.12 nM for H7-767. The EC₅₀ for binding to activated cynomolgus CD3 CD4+ T cells was 0.09 nM for 2H7-hIgG4 and 0.04 nM for 2H7-hIgG4-df-hIL-2 (T3A/D20A/R38E/C125A). EC₅₀ for binding to activated cynomolgus CD3⁺CD8+ T cells was 0.08 nM for 2H7-hIgG4 and 0.03 nM for 2H7-hIgG4-df-hIL-2 (T3A/D20A/R38E/C125A). The EC₅₀ for binding to activated cynomolgus CD3⁺CD4⁺ T cells was 0.26 nM and activated cynomolgus CD3 CD8⁺ T cells was 0.24 nM for H7-767. This data demonstrated that when the hPD-1 antibodies were converted to anti-hPD-1-attenuated hIL-2 fusion proteins, the calculated EC₅₀ value for binding to activated hPD-1 remained similar to the calculated EC₅₀ value of hPD-1 naked antibody binding to hPD-1.

H7-767 and H7-632-hIgG1-LAGA anti-PD-1 naked antibody were tested for binding on primary non-activated human CD4⁺ and CD8⁺ T cells by flow cytometry. Frozen human CD3⁺ T were thawed and flow cytometry performed as described above. Both H7-767 and H7-632-hIgG1-LAGA anti-PD-1 naked antibody did not bind non-activated human CD4⁺ and CD8⁺ T cells (data not shown).

### Example 13: Quantification of Binding of Anti-hPD-1 Antibodies and Anti-hPD-1-Attenuated hIL-2 Fusion Proteins to Recombinant Human or Cynomolgus PD-1 by Surface Plasmon Resonance (SPR)

Surface plasmon resonance binding analysis was performed using a high-throughput SPR Carterra® LSA™ to determine binding affinities of anti-hPD-1 antibodies and anti-hPD-1-attenuated hIL-2 fusion proteins. Proteins were diluted to 2 or 10 μg/mL in 10 mM sodium acetate pH 4.5 containing 0.01% Tween-20 and coupled to a HC30M (Carterra Bio) chip using sulpho-N-hydroxysuccinimide/1-ethyl-3-(3-dimethylamino) propyl carbodiimide (sulpho-NHS/EDC) coupling chemistry and blocked with ethanolamine. A non-regenerative kinetic coupling process was used to determine binding kinetics to commercially sourced recombinant His-tagged human PD-1 and His-tagged cynomolgus PD-1 (Acro Biosystems).

Anti-hPD-1 antibodies and anti-hPD-1-attenuated hIL-2 fusion proteins were expressed with either a modified human IgG1 or a modified IgG4 isotype with a kappa light chain framework. Additional substitutions L235E, or L235A/G237A (LAGA, as described in Int'l Pub. No. WO1998/006248) (numbering based upon the EU numbering system) were introduced to the Fc region to abrogate effector functions of the immunoglobulin component.

The association constants (kₐ), dissociation constants (k_{d}), and equilibrium constants (K_{D}) of various anti-hPD-1 antibodies and anti-hPD-1 antibody-attenuated hIL-2 fusion proteins binding to recombinant human or cynomolgus PD-1 proteins was determined from the titration curves and the Carterra Kinetics software. The maximal feasible SPR signal generated (Rₘₐₓ) and residual standard deviation (Res SD) was also calculated. The results from the kinetics screen are summarized in Table 21, and demonstrated that the addition of the attenuated hIL-2 moiety on anti-hPD-1 antibodies did not modulate PD-1 antibody binding to the human PD-1 or cynomolgus PD-1 antigens. In a separate experiment, H7-632-hIgG1-LAGA (SEQ ID NOs: 414 and 415) was measured by SPR and had a steady state equilibrium dissociation constant (K_{D}) of 1.23×10⁻⁹M and H7-767 had a K_{D}=1.93×10⁻⁹M.

TABLE 21
Binding kinetics of anti-hPD-1 and anti-hPD-1-hIL-2 fusion proteins to recombinant
human PD-1 and cynomolgus PD-1 by high-throughput SPR Carterra ® LSA ™

| | | Kinetics Summary | | | | | | | | | |
|---|---|---|---|---|---|---|---|---|---|---|---|
| | | Human PD-1 | | | | | Cyno PD-1 | | | | |
| | | kₐ | k_{d} | K_{D} | | Res | kₐ | k_{d} | K_{D} | | Res |
| | Name | (M−1 s−1) | (s−1) | (M) | Rmax | SD | (M−1 s−1) | (s−1) | (M) | Rmax | SD |
| Anti-hPD-1 | 2H7-hIgG4-LE | 2.60E+05 | 8.20E−04 | 3.10E−09 | 146 | 8.5 | 1.40E+05 | 1.00E−03 | 7.10E−09 | 186 | 6.5 |
| Antibodies | 2H7-hIgG4-LAGA | 1.70E+05 | 9.10E−04 | 5.30E−09 | 258 | 13 | 9.10E+04 | 1.10E−03 | 1.20E−08 | 295 | 11 |
| | Abz1mod-hIgG4 | 1.40E+05 | 6.00E−05 | 4.26E−10 | 123 | 8.1 | 1.70E+05 | 6.20E−04 | 3.70E−09 | 183 | 10 |
| | A2-hIgG4 | 3.70E+04 | 1.30E−03 | 3.60E−08 | 341 | 7.8 | 6.80E+04 | 4.00E−02 | 5.89E−07 | 238 | 4.8 |
| | OMC.1.B6-hIgG4 | 7.70E+04 | 4.00E−03 | 5.20E−08 | 241 | 6.8 | 8.80E+04 | 4.40E−03 | 5.10E−08 | 262 | 5.5 |
| | OMC.2.C6-hIgG4 | 5.70E+04 | 6.60E−04 | 1.20E−08 | 229 | 8.7 | 5.50E+04 | 7.40E−04 | 1.30E−08 | 268 | 8.9 |
| | OMC.1.D6-hIgG4 | 4.10E+04 | 1.70E−03 | 4.20E−08 | 263 | 5.1 | 5.80E+04 | 4.00E−02 | 6.94E−07 | 182 | 4 |
| | OMC476pH7-hIgG4 | 6.00E+04 | 1.10E−03 | 1.80E−08 | 225 | 8.9 | 7.80E+04 | 1.70E−02 | 2.21E−07 | 254 | 2.8 |
| | OMC476pB11-hIgG4 | 5.00E+04 | 1.50E−03 | 3.00E−08 | 221 | 7 | 8.00E+04 | 2.60E−02 | 3.21E−07 | 204 | 2.7 |
| | OMC476pG10-hIgG4 | 9.30E+04 | 4.10E−03 | 4.40E−08 | 256 | 4.9 | 1.20E+05 | 6.00E−02 | 4.87E−07 | 188 | 4.9 |
| | OMC476pH10-hIgG4 | 1.00E+05 | 6.50E−03 | 6.40E−08 | 63 | 1.8 | 1.20E+05 | 5.90E−02 | 4.80E−07 | 81 | 3.8 |
| | OMC476pE4-hIgG4 | 7.90E+04 | 8.80E−04 | 1.10E−08 | 216 | 9.6 | 1.00E+05 | 3.50E−02 | 3.31E−07 | 204 | 6.3 |
| | D12-hIgG4 | 5.70E+04 | 4.60E−04 | 8.10E−09 | 234 | 9.9 | 5.90E+04 | 1.60E−02 | 2.72E−07 | 297 | 2.4 |
| | G12-hIgG4 | 5.30E+04 | 2.60E−03 | 5.00E−08 | 477 | 8.5 | 8.60E+04 | 6.50E−02 | 7.54E−07 | 327 | 7.8 |
| | EH12.2H7-mIgG1* | 1.10E+05 | 2.20E−03 | 1.90E−08 | 309 | 5.2 | 8.60E+04 | 7.30E−03 | 8.50E−08 | 307 | 4.2 |
| | J105-mIgG1* | 6.20E+04 | 5.30E−03 | 8.60E−08 | 186 | 2.8 | 5.00E+04 | 6.60E−02 | 1.30E−09 | 92 | 1.2 |
| | MIH4-mIgG1* | 1.30E+05 | 1.40E−03 | 1.00E−08 | 117 | 3.8 | 1.30E+05 | 1.20E−01 | 8.91E−07 | 61 | 1.8 |
| | J110-hIgG1 | 1.00E+05 | 1.00E−03 | 1.00E−08 | 346 | 15 | 4.10E+04 | 5.00E−02 | 1.20E−09 | 228 | 8.2 |
| | OPDIVO ® (nivolumab) | 1.70E+05 | 1.70E−03 | 1.00E−08 | 55 | 2.6 | 1.50E+05 | 9.50E−04 | 6.40E−09 | 75 | 3.2 |
| | KEYTRUDA ® (pembrolizumab) | 2.90E+05 | 1.30E−03 | 4.50E−09 | 19 | 2.6 | 7.00E+05 | 4.00E−04 | 5.75E−07 | 53 | 3.7 |
| Anti- | 2H7-hIgG1-LAGA-hIL-2 | 1.50E+05 | 8.30E−04 | 5.40E−09 | 420 | 28 | 8.80E+04 | 9.70E−04 | 1.10E−08 | 502 | 21 |
| hPD-1- | (T3A/D20A/R38E/C125A) | | | | | | | | | | |
| attenuated | 2H7-hIgG4-LE-hIL-2 | 1.90E+05 | 8.10E−04 | 4.30E−09 | 374 | 17 | 9.00E+04 | 1.00E−03 | 1.10E−08 | 449 | 15 |
| hIL-2 | (T3A/D20A/R38E/C125A) | | | | | | | | | | |
| Fusion | 2H7-hIgG4-LAGA-hIL-2 | 1.70E+05 | 8.20E−04 | 4.80E−09 | 359 | 22 | 9.60E+04 | 9.80E−04 | 1.00E−08 | 429 | 17 |
| Proteins | (T3A/D20A/R38E/C125A) | | | | | | | | | | |
| | 2H7-hIgG1-LAGA-hIL-2 | 1.80E+05 | 7.80E−04 | 4.30E−09 | 417 | 25 | 9.10E+04 | 9.70E−04 | 1.10E−08 | 505 | 19 |
| | (T3A/R38E/I92K/C125A) | | | | | | | | | | |
| | hIgG4-LE-hIL-2 | 1.90E+05 | 8.80E−04 | 4.70E−09 | 380 | 19 | 9.50E+04 | 1.10E−03 | 1.20E−08 | 429 | 17 |
| | (T3A/R38E/I92K/C125A) | | | | | | | | | | |
| | 2H7-hIgG4-LAGA-hIL-2 | 2.20E+05 | 8.20E−04 | 3.70E−09 | 355 | 17 | 1.00E+05 | 1.10E−03 | 1.10E−08 | 418 | 16 |
| | (T3A/R38E/I92K/C125A) | | | | | | | | | | |
| | 2H7-hIgG1-LAGA-hIL-2 | 1.30E+05 | 8.10E−04 | 6.20E−09 | 458 | 29 | 7.60E+04 | 1.00E−03 | 1.30E−08 | 532 | 23 |
| | (T3A/R38E/D84K/C125A) | | | | | | | | | | |
| | 2H7-hIgG4-LE-hIL-2 | 2.00E+05 | 9.40E−04 | 4.70E−09 | 232 | 13 | 1.30E+05 | 1.10E−03 | 8.20E−09 | 262 | 10 |
| | (T3A/R38E/D84K/C125A) | | | | | | | | | | |
| | 2H7 hIgG4LAGA-df-hIL-2 | 1.70E+05 | 7.30E−04 | 4.40E−09 | 400 | 20 | 8.10E+04 | 1.00E−03 | 1.30E−08 | 482 | 20 |
| | (T3A/R38E/D84K/C125A) | | | | | | | | | | |

*Commercially sourced, no sequence available

### Example 14: Determining Whether Anti-hPD-1 Antibodies and Anti-hPD-1-Attenuated hIL-2 Fusion Proteins Compete with Anti-hPD-1 #1 and Anti-hPD-1 #2 for Binding to PD-1 by Surface Plasmon Resonance (SPR)

Anti-hPD-1 and anti-hPD-1-attenuated hIL-2 fusion proteins were assayed for competition with one another using a sandwich method. Antibodies and corresponding antibody-IL-2 cytokine-fusion proteins were immobilized to HC30M chips using amine coupling chemistry described in Example 13. Following kinetic analysis described in Example 13, 80 nM human PD-1 (Acro Biosystems, Cat #PD-1-H5221-100 ug) was injected into the whole array. Competing anti-hPD-1 and anti-hPD-1-attenuated hIL-2 fusion proteins (analyte) were diluted to 30 μg/mL and subsequently injected into the array and binding parameters were assessed using SPR. Assessment of all anti-hPD-1 and anti-hPD-1-hIL-2 fusion proteins was performed in duplicate. Some variants tested had a modified human IgG1 or IgG4 kappa light chain framework with additional L235E or L235A/G237A (LAGA) substitutions to abrogate effector function of the immunoglobulin.

The screening of pairs of anti-hPD-1 or anti-hPD-1-attenuated hIL-2 fusion proteins allowed the identification of two bins, shown in Table 22. Antibodies and fusion proteins from Group 1 were able to bind hPD-1 in the presence of all antibodies and fusion proteins from Group 2, but competed with all members of the same Group. Antibodies and fusion proteins from Group 2 were able to bind hPD-1 in the presence of all antibodies and fusion proteins from Group 1, but competed with all members of the same Group. None of the anti-hPD-1 listed in Group 1 in Table 22 competed with KEYTRUDA® and OPDIVO®.

TABLE 22
Groups 1 and 2 from anti-hPD-1 and anti-hPD-1-attenuated
hIL-2 fusion protein binning screen by SPR

| Group 1 | Group 2 |
|---|---|
| Abz1mod-hIgG4 | KEYTRUDA ® |
| OMC.1.B6-hIgG4 | OPDIVO ® |
| OMC.1.D6-hIgG4 | Anti-hPD-1 clone |
| OMC.2.C6-hIgG4 | EH12.2H7-mIgG1* |
| OMC476pE4-hIgG4 | Anti-hPD-1 clone |
| OMC476pH7-hIgG4 | J105-mIgG1* |
| OMC476pB11-hIgG4 | |
| OMC476pH10-hIgG4 | |
| OMC476pG10-hIgG4 | |
| A2-hIgG4 | |
| D12-hIgG4 | |
| G12-hIgG4 | |
| 2H7-hIgG4-LE | |
| 2H7-hIgG4-LE-df-hIL-2 (T3A/D20A/R38E/C125A) | |
| 2H7-hIgG4-LAGA-df-hIL-2 | |
| (T3A/D20A/R38E/C125A) | |
| 2H7-hIgG1-LAGA-df-hIL-2 | |
| (T3A/D20A/R38E/C125A) | |
| 2H7-hIgG4-LE-df-hIL-2 (T3A/R38E/I92K/C125A) | |
| 2H7-hIgG4-LAGA-df-hIL-2 | |
| (T3A/R38E/I92K/C125A) | |
| 2H7-hIgG1-LAGA-df-hIL-2 | |
| (T3A/R38E/I92K/C125A) | |
| 2H7-hIgG4-LE-df-hIL-2 (T3A/R38E/D84K/C125A) | |
| 2H7-hIgG4-LAGA-df-hIL-2 | |
| (T3A/R38E/D84K/C125A) | |
| 2H7-hIgG1-LAGA-df-hIL-2 | |
| (T3A/R38E/D84K/C125A) | |
| Anti-PD-1 clone MIH4 mIgG1 | |
| Anti-PD-1 clone J110 hIgG1 | |

*Commercially sourced, no sequence available

### Example 15: Antagonism of Anti-hPD-1-Attenuated hIL-2 Fusion Proteins to hPD-1 in the Presence of Anti-hPD-1 #1 and Anti-hPD-1 #2

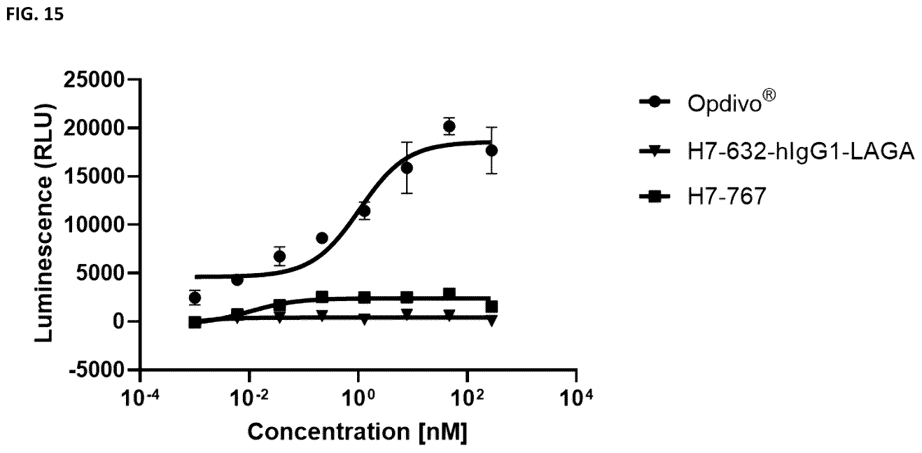

Anti-hPD-1-attenuated hIL-2 fusion proteins were tested for antagonism of hPD-1. Characterization of anti-hPD-1-attenuated hIL-2 fusion proteins was performed according to General Methods Protocol C. FIG. 7 illustrates these results. When compared to the PD-1 antagonists KEYTRUDA® or OPDIVO®, 2H7-hIgG4-df-hIL-2 (D20A/R38E), C51E6-5-hIgG4-L6-hIL-2 (D20A/R38E), and A2-hIgG4-df-hIL-2 (D20A/R38E) were non-antagonistic to human PD-1, as demonstrated by the low level of detectable luminescence. H7-632-hIgG1-LAGA and H7-767 were also tested for antagonist activity as described in General Protocol C. FIG. 15 illustrates that H7-632-hIgG1-LAGA and H7-767 do not block hPD-L1 (SEQ ID NO: 584) from interacting with the hPD-1 receptor.

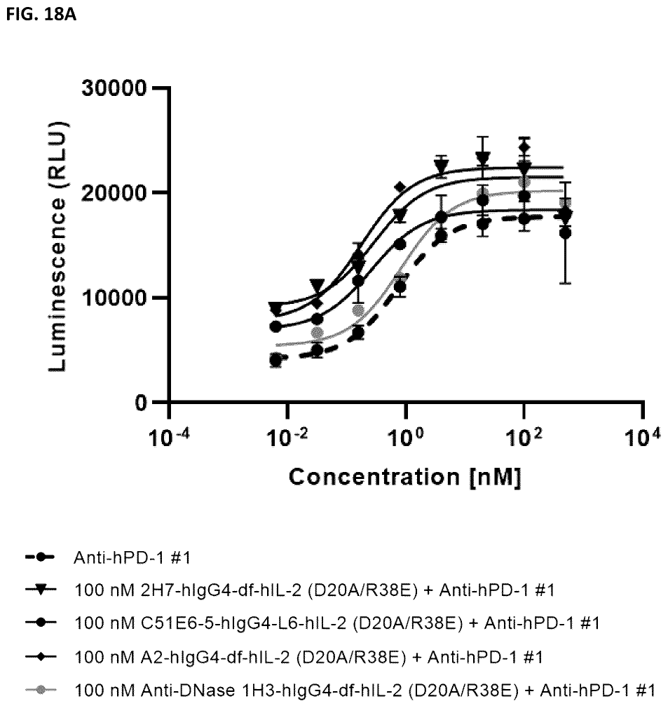

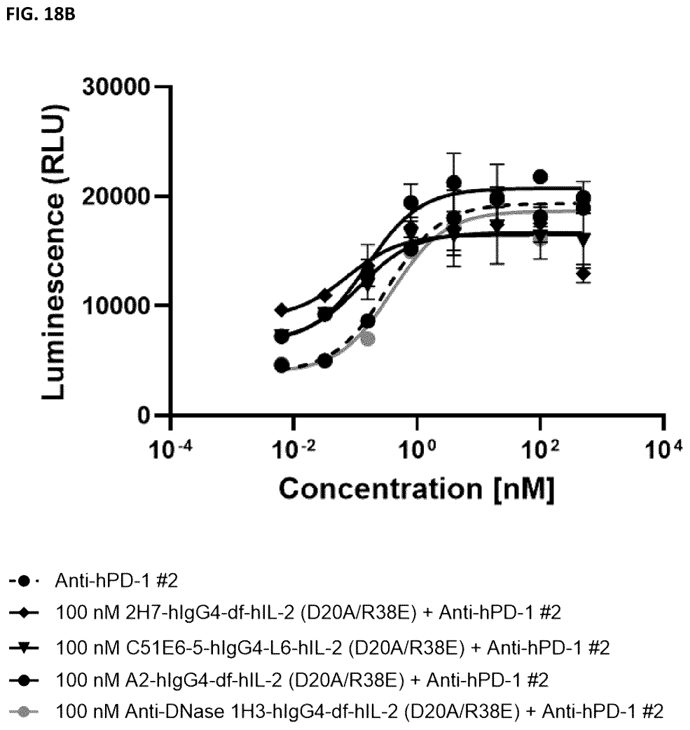

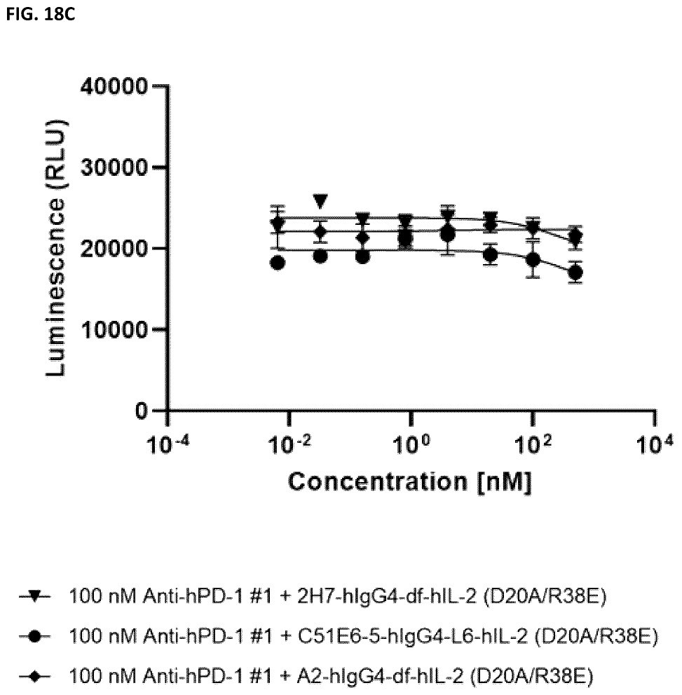

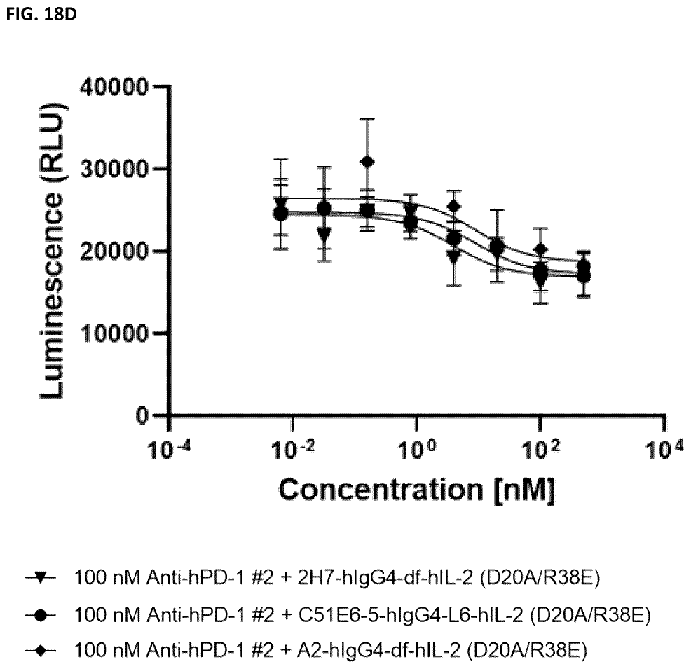

For competition assays using the cell-based co-culture assay described in General protocol C, a few modifications were performed. Samples of the anti-hPD-1-attenuated hIL-2 fusion proteins were diluted to a fixed concentration of 400 nM and 20 μL was added to 20 μL of titrated anti-hPD-1 #1 or anti-hPD-1 #2. The 40 μL mixture was added to CHO cells. Forty (40) μL of Jurkat PD-1 effector cells were overlayed on the mixture of CHO cells and anti-hPD-1-attenuated hIL-2 fusion proteins. In this competition assay, a final concentration of saturating 100 nM anti-hPD-1-attenuated hIL-2 fusion proteins was tested in combination with titrated anti-hPD-1 #1 or anti-hPD-1 #2. The rest of the assay was performed as described in General Protocol C. FIG. 18A and FIG. 18B demonstrate that the addition of 100 nM anti-hPD-1-attenuated hIL-2 fusion proteins did not compete with the blocking of titrated anti-hPD-1 #1 binding to hPD-L1 (SEQ ID NO: 584). Dose-titration curves of anti-hPD-1 #1 remained unchanged from curves without competitor antibody, suggesting that the presence of anti-hPD-1-attenuated hIL-2 fusion proteins did not compete with anti-hPD-1 #1 function even at high concentrations. In the presence of 100 nM 2H7-hIgG4-df-hIL-2 (D20A/R38E) and 100 nM C51E6-5-hIgG4-L6-hIL-2 (D20A/R38E), anti-hPD-1 #2 exhibited a 35% reduction in luminescence (RLU) at higher concentrations of anti-hPD-1 #2 (FIG. 18B) but it is unclear if this reduction was significant due to the extent of the standard deviation.

In the converse experiment, either anti-hPD-1 #1 or anti-hPD-1 #2 were diluted to a concentration of 400 nM and 20 μL was combined with 20 μL of titrated anti-hPD-1-attenuated hIL-2 fusion proteins. Anti-hPD-1-attenuated hIL-2 fusion proteins were serially titrated and the 40 μL mixture was added to CHO cells, then overlayed with 40 μL of Jurkat PD-1 Effector cells. The rest of the assay was performed as described in General Protocol C. FIG. 18C and FIG. 18D demonstrate that the addition of 100 nM anti-hPD-1 #1 (FIG. 18C) or 100 nM anti-hPD-1 #2 (FIG. 18D) do not impair the ability of the anti-hPD-1-attenuated hIL-2 fusion proteins to be antagonists. The observed flat curve above 18,000 relative luminescent units (RLU) indicated that there was no competition for antagonist activity and the anti-hPD-1-attenuated hIL-2 fusion proteins tested remained able to exhibit antagonist function even in the presence of anti-hPD-1 #1 or anti-hPD-1 #2.

### Example 16: Testing Anti-hPD-1-Attenuated hIL-2 Fusion Proteins for Attenuation on the High-Affinity and Intermediate-Affinity hIL-2 Receptors with Cell-Based Proliferation Assays

Anti-hPD-1-attenuated hIL-2 fusion proteins were evaluated for the level of attenuation of hIL-2 activity using the cell proliferation assays on NK-92 and TF1+IL-2Rβ cell lines as described in General Protocol E. Control fusion proteins included fusion proteins incorporating an anti-DNase I antibody (designated 1H3) with a human IgG4 or human IgG1 backbone directly fused to hIL-2 or with a linker (SEQ ID NO: 355) to demonstrate the effects of non-targeting attenuated hIL-2 fusion proteins. The hIL-2 sequence of these constructs contained substitutions for attenuated hIL-2 activity as described in Example 2. Full, partial, or no agonistic IL-2 activity (inactive) was also assessed similarly to Example 3. Some of the variants tested were expressed on a modified human IgG1 or IgG4 isotype with a kappa light chain, with additional L235E or L235A/G237A (LAGA) substitutions in the Fc region to abrogate immunoglobulin effector function. In some antibody-cytokine fusion proteins, the hIL-2 cytokine was fused to the C-terminus of the light chain (LC fusion).

The calculated EC₅₀ of each antibody-cytokine fusion protein was determined from relative luminescence units (RLU), and fold change EC₅₀ was calculated when compared with recombinant human IL-2 (rhIL-2). The fold change from rhIL-2 and agonistic activity is summarized in Table 23. Agonistic activity was measured as full, partial, or inactive as determined by the maximal luminescence of antibody-attenuated hIL-2 fusion proteins in comparison to the maximal luminescence of rhIL-2. Antibody-attenuated hIL-2 fusion proteins dose-titration curves that reached the maximal luminescence as the rhIL-2 were considered to be variants with full activity. Partial activity was calculated as a percentage of full activity using rhIL-2 maximal luminescence as 100%. Maximal RLU of antibody-attenuated hIL-2 fusion proteins with less than 10% of the rhIL-2 maximal RLU at the highest concentration of 1200 nM were considered to have no agonist activity or inactive. For some variants EC₅₀ values were estimated only since maximal luminescence was not reached, as annotated by an ^{a} in Table 23.

TABLE 23
Fold change from rhIL-2 and agonistic activity of antibody-attenuated hIL-2 fusion proteins
on NK-92 (high-affinity IL-2R) and TF1 + IL-2Rβ (intermediate-affinity IL-2R) cell lines.

| | | Fold change | Agonistic | Fold change | Agonistic |
|---|---|---|---|---|---|
| | | from rhIL-2 | Activity | from rhIL-2 | Activity |
| | Variants | (NK-92) | (NK-92) | (TF1 + IL-2Rβ) | (TF1 + IL-2Rβ) |
| Non-Targeted | 1H3-hIgG4-df-hIL-2 (WT) | 0 ^{a} | Full | 0-1 | Full |
| Antibody- | 1H3-hIgG4-L6-hIL-2 (WT) | 0 ^{a} | Full | 0-1 | Full |
| Attenuated | 1H3-hIgG4-df-hIL-2 (WT) LC fusion | 0 ^{a} | Full | 24 | Full |
| hIL-2 | 1H3-hIgG4-L6-hIL-2 (WT) LC fusion | 0 ^{a} | Full | 2 | Full |
| Fusion | 1H3-hIgG4-L6-hIL-2 (D20Y) | >10,000 on | Inactive | >10,000 on | Inactive |
| Proteins | | graph, NC ^{a} | | graph, NC ^{a} | |
| | 1H3-hIgG4-df-hIL-2 (D20Y) | >10,000 on | Partial, 60% | >10,000 on | Inactive |
| | | graph, NC ^{a} | | graph, NC ^{a} | |
| | 1H3-hIgG1-df-hIL-2 (D20Y) | 8539 ^{a} | Partial, 90% | >10,000 on | Inactive |
| | | | | graph, NC ^{a} | |
| | 1H3-hIgG4-L6-hIL-2 (D20A/R38P) | >10,000 ^{a} | Partial, 80% | 4132 ^{a} | Full |
| | 1H3-hIgG4-L6-hIL-2 (D20A/R38S) | >10,000 on | Partial, 90% | 9225 ^{a} | Full |
| | | graph, NC ^{a} | | | |
| | 1H3-hIgG4-L6-hIL-2 (D20A/R38D) | 118 ^{a} | Partial, 90% | 8591 ^{a} | Partial, 90% |
| | 1H3-hIgG4-L6-hIL-2 (D20A/R38Q/E95A) | 153 | Full | 5738 ^{a} | Full |
| | 1H3-hIgG4-L6-hIL-2 (D20A/F42H/E95A) | >10,000 ^{a} | Full | 1368 ^{a} | Full |
| | 1H3-hIgG4-L6-hIL-2 (R38D/I92D) | 190 | Full | 437 | Full |
| | 1H3-hIgG4-L6-hIL-2 (R38E/I92D) | 377 ^{a} | Full | 296 | Full |
| | 1H3-hIgG4-L6-hIL-2 (F42H/I92D) | 794 ^{a} | Full | 393 | Full |
| | 1H3-hIgG4-df-hIL-2 (D20A/R38E) | 868 ^{a} | Partial, | >10,000 ^{a} | Partial, |
| | | | 90%-Full | | 70%-Full |
| | 1H3-hIgG4-L6-hIL-2 (D20A/R38E) | 177 ^{a} | Partial, | >10,000 ^{a} | Partial, |
| | | | 60-80% | | 70%-Full |
| | 1H3-hIgG4-L6-hIL-2 (T3A/D20A/R38E) | >10,000 on | Partial, 40% | >10,000 | Partial, 70% |
| | | graph, NC ^{a} | | | |
| | 1H3-hIgG4-L6-hIL-2 (D20A/R38E/C125A) | >10,000 ^{a} | Partial, 20% | 6436 | Partial, 40% |
| | 1H3-hIgG4-L6-hIL-2 | >10,000 on | Partial, 20% | >10,000 | Full |
| | (T3A/D20A/R38E/C125A) | graph, NC ^{a} | | | |
| | 1H3-hIgG1-L6-hIL-2 (D20A/R38E) | NT | NT | 250-372 | Full |
| | 1H3-hIgG1-L6-hIL-2(T3A/D20A/R38E) | 392 | Partial, 80% | 186 | Partial, 60% |
| | 1H3-hIgG1-L6-hIL-2 (D20A/R38E/C125A) | 2346 ^{a} | Partial, 80% | 2157 ^{a} | Partial, 60% |
| | 1H3-hIgG4-df-hIL-2 (D20A/R38E) LC | >10,000 on | Inactive | >10,000 on | Inactive |
| | fusion | graph, NC ^{a} | | graph, NC ^{a} | |
| | 1H3-hIgG4-L6-hIL-2 (D20A/R38E) LC | >10,000 on | Inactive | 2155 ^{a} | Partial, 30% |
| | fusion | graph, NC ^{a} | | | |
| | 1H3-hIgG1-L6-hIL2 (H16A) | 0 | Full | 0 ^{a} | Full |
| | 1H3-hIgG1-L6-hIL2 (F42A) | 0 | Full | 0 ^{a} | Full |
| | 1H3-hIgG1-L6-hIL2 (H16A/F42A) | 1 | Full | 0 ^{a} | Full |
| Anti-hPD-1- | A2-hIgG4-df-hIL-2 (D20A/R38E) | 811 ^{a} | Partial, | >10,000 ^{a} | Partial, |
| Attenuated | | | 20-90% | | 60-80% |
| hIL-2 | D12-hIgG4-df-hIL-2 (D20A/R38E) | >10,000 on | Partial, 20% | >10,000 ^{a} | Partial, 40% |
| Fusion | | graph, NC ^{a} | | | |
| Proteins | G12-hIgG4-df-hIL-2 (D20A/R38E) | >10,000 ^{a} | Partial, 20% | >10,000 ^{a} | Partial, 50% |
| | OMC476pB11-hIgG4-df-hIL-2 | >10,000 on | Partial, 70% | 36 | Full |
| | (D20A/R38E) | graph, NC ^{a} | | | |
| | OMC476pE4-hIgG4-df-hIL-2 | >10,000 on | Partial, 70% | 1619 | Full |
| | (D20A/R38E) | graph, NC ^{a} | | | |
| | OMC476pG10-hIgG4-df-hIL-2 | >10,000 ^{a} | Partial, 70% | NC ^{a} | Inactive |
| | (D20A/R38E) | | | | |
| | OMC476pH10-hIgG4-df-hIL-2 | >10,000 on | Partial, 70% | 3563 ^{a} | Partial, 80% |
| | (D20A/R38E) | graph, NC ^{a} | | | |
| | A2-hIgG4-df-hIL-2 (D20A/F42A) | 284 | Full | 4323 ^{a} | Partial, 80% |
| | A2-hIgG4-df-hIL-2 (D20A/F42S) | 3542 | Full | 4052 ^{a} | Partial, 90% |
| | A2-hIgG4-df-hIL-2 (D20S/R38E) | NT | NT | 7035 ^{a} | Full |
| | A2-hIgG4-df-hIL-2 (F42A/N88R) | 6423 | Full | 3757 ^{a} | Partial, 90% |
| | A2-hIgG4-df-hIL-2 (F42I/I92D) | 9543 | Full | 5611 ^{a} | Partial, 90% |
| | A2-hIgG4-df-hIL-2 (F42Q/I92D) | 8572 | Full | 3363 ^{a} | Full |
| | A2-hIgG4-df-hIL-2 (F42T/I92D) | 2175 | Full | 5649 ^{a} | Full |
| | A2-hIgG4-df-hIL-2 (F42W/I92D) | 1239 | Full | 4409 ^{a} | Partial, 50% |
| | A2-hIgG4-df-hIL-2 (R38E/D84K) | 160-1503 | Full | 1158-1716 | Partial, 90% |
| | A2-hIgG4-df-hIL-2 (R38E/I92K) | 252-977 | Full | 864-1655 | Partial, |
| | | | | | 80%-Full |
| | C51E6-5-hIgG4-df-hIL-2 (D20A/R38E) | 4317 | Partial, 70% | >10,000 ^{a} | Partial, 60% |
| | C51E6-5-hIgG4-L6-hIL-2 (D20A/R38E) | NT | NT | >10,000 ^{a} | Partial, 80% |
| | C51E6-5-hIgG4-LE-df-hIL-2 | 4326 | Partial, 80% | >10,000 ^{a} | Partial, 70% |
| | (T3A/D20A/R38E/C125A) | | | | |
| | C51E6-5-hIgG4-LAGA-df-hIL-2 | 4336 | Partial, 60% | >10,000 on | Partial, 60% |
| | (T3A/D20A/R38E/C125A) | | | graph, NC ^{a} | |
| | OMC.1.B6-hIgG4-L6-hIL-2 (D20A/R38E) | NT | NT | 8460 ^{a} | Partial, 70% |
| | OMC.1.D6-hIgG4-L6-hIL-2 (D20A/R38E) | NT | NT | >10,000 ^{a} | Partial, 70% |
| | OMC.2.C6-hIgG4-L6-hIL-2 (D20A/R38E) | NT | NT | >10,000 ^{a} | Partial, 70% |
| | 2A3.H7-hIgG4-df-hIL-2 (D20A/R38E) | NT | NT | 6603 ^{a} | Partial, 60% |
| | 1H9-hIgG4-df-hIL-2 (D20A/R38E) | NT | NT | 9769 ^{a} | Partial, 90% |
| | 1D5-hIgG4-df-hIL-2 (D20A/R38E) | NT | NT | 7420 ^{a} | Partial, 80% |
| | 1D5-hIgG4-LE-df-hIL-2 | NT | NT | NC ^{a} | Inactive |
| | (T3A/D20A/R38E/C125A) | | | | |
| | 1D5-hIgG4-LAGA-df-hIL-2 | NT | NT | NC ^{a} | Partial, 20% |
| | (T3A/D20A/R38E/C125A) | | | | |
| | 2H7-hIgG1-df-hIL-2 | 4839 ^{a} | Full | 2057 ^{a} | Full |
| | (T3A/D20A/R38E/C125A) | | | | |
| | 2H7-hIgG1-LE-df-hIL-2 | 7727 ^{a} | Full | 6729 ^{a} | Partial, 80% |
| | (T3A/D20A/R38E/C125A) | | | | |
| | 2H7-hIgG1-LAGA-df-hIL-2 | >10,000 ^{a} | Full | 3428 ^{a} | Partial, |
| | (T3A/D20A/R38E/C125A) | | | | 50-60% |
| | 2H7-hIgG4-df-hIL-2 | 707-7206 ^{a} | Full | >10,000 ^{a} | Partial, |
| | (T3A/D20A/R38E/C125A) | | | | 60%-Full |
| | 2H7-hIgG4-LE-df-hIL-2 | >10,000 | Full | >10,000 ^{a} | Partial, |
| | (T3A/D20A/R38E/C125A) | | | | 50-60% |
| | 2H7-hIgG4-LAGA-df-hIL-2 | >10,000 | Full | 7480 ^{a} | Partial, |
| | T3A/D20A/R38E/C125A) | | | | 40-50% |
| | H7-767 | >10,000 ^{a} | Partial, Full | >10,000 ^{a} | Full |
| | 2H7-hIgG1-df-hIL-2 | 1500 | Partial, 90% | 140 ^{a} | Full |
| | (T3A/R38E/D84K/C125A) | | | | |
| | 2H7-hIgG1-LE-df-hIL-2 | 1268 | Partial, 90% | 517 ^{a} | Full |
| | (T3A/R38E/D84K/C125A) | | | | |
| | 2H7-hIgG1-LAGA-df-hIL-2 | 2157-4035 | Partial, | 774-1650 ^{a} | Full |
| | (T3A/R38E/D84K/C125A) | | 90%-Full | | |
| | 2H7-hIgG4-df-hIL-2 | 1602 | Partial, 90% | >10,000 ^{a} | Full |
| | (T3A/R38E/D84K/C125A) | | | | |
| | 2H7-hIgG4-LE-df-hIL-2 | 1675-5096 | Partial, 90% | 1281-2842 ^{a} | Full |
| | (T3A/R38E/D84K/C125A) | | | | |
| | 2H7-hIgG4-LAGA-df-hIL-2 | 1596-5689 | Partial, | 1203-3515 ^{a} | Partial, |
| | (T3A/R38E/D84K/C125A) | | 90%-Full | | 60-80% |
| | 2H7-hIgG1-df-hIL-2 | 370 | Full | 160 | Full |
| | (T3A/R38E/I92K/C125A) | | | | |
| | 2H7-hIgG1-LE-df-hIL-2 | 319 | Full | 656 | Full |
| | (T3A/R38E/I92K/C125A) | | | | |
| | 2H7-hIgG1-LAGA-df-hIL-2 | 406-1280 | Full | 514-1569 ^{a} | Full |
| | (T3A/R38E/I92K/C125A) | | | | |
| | 2H7-hIgG4-df-hIL-2 | 520 | Full | 789-926 | Partial, |
| | (T3A/R38E/I92K/C125A) | | | | 80%-Full |
| | 2H7-hIgG4-LE-df-hIL-2 | 610-1675 | Full | 474-2080 ^{a} | Full |
| | (T3A/R38E/I92K/C125A) | | | | |
| | 2H7-hIgG4-LAGA-df-hIL-2 | 827-2888 | Full | 737-2845 ^{a} | Partial, |
| | (T3A/R38E/I92K/C125A) | | | | 70%-Full |
| | 2H7-hIgG1-LAGA-df-hIL2 | 6689 | Full | 9711 ^{a} | Partial, 70% |
| | (T3A/D20S/R38E/C125A) | | | | |
| | 2H7-hIgG1-LAGA-df-hIL2 | 6199 | Full | 3915 | Full |
| | (T3A/R38E/D84F/C125A) | | | | |
| | 2H7-hIgG1-LAGA-df-hIL2 | 75 | Full | 89 | Full |
| | (T3A/R38E/I92R/C125A) | | | | |
| | 2H7-hIgG1-LAGA-df-hIL2 | 118 | Full | 53 | Full |
| | (T3A/R38E/I92E/C125A) | | | | |
| | 2H7-hIgG1-LAGA-df-hIL2 | 9 | Full | 30 | Full |
| | (T3A/R38E/I92S/C125A) | | | | |
| | 2H7-hIgG1-LAGA-df-hIL2 | 2717 | Full | 3396 ^{a} | Partial, 80% |
| | (T3A/R38E/I92D/C125A) | | | | |
| | 2H7-hIgG1-LAGA-df-hIL2 | 126 | Full | 122 | Full |
| | (T3A/H16E/R38E/C125A) | | | | |

NT = Not Tested
NC = Not Calculated by GraphPad Prism 7
^{a} = Fold change is an estimate only since a full four parameter logistic curve was not reached

### Example 17: Rescue of IL-2 Activity of Anti-hPD-1-Attenuated hIL-2 Fusion Proteins on a Cell Line Expressing the Intermediate-Affinity hIL-2 Receptor and hPD-1

Anti-hPD-1-attenuated hIL-2 fusion proteins were evaluated for rescue of hIL-2 activity using a targeted cell line expressing hPD-1. Briefly, the TF1+IL-2Rβ cell line described in General Methods Protocol D was modified through lentiviral transduction to express the hPD-1 receptor (SEQ ID NO: 580). Flow cytometry with a Brilliant Blue 515 conjugated hPD-1 antibody (BD Biosciences Cat #565936) was used to detect hPD-1 expressing TF1+IL-2Rβ cells. Cells were sorted for low hPD-1 expression (less than 103 intensity on the Brilliant Blue 515 fluorophore). The pool was sorted twice more to collect cells that approximated hPD-1 expression levels on activated primary cells. This cell line (TF1+IL-2Rβ+hPD-1) was expanded and frozen in aliquots for the cell-based proliferation assays. Proliferation assays were performed as described in General Methods Protocol E with an incubation period of 3 days. Some variants tested had a modified human IgG1 or IgG4 kappa light chain framework with additional L235E or L235A/G237A (LAGA) substitutions to abrogate effector function of the immunoglobulin.

Table 24 summarizes the results from the proliferation assays on the targeted TF1+IL-2Rβ+hPD-1 cell line. Agonistic activity was measured as full, partial, or inactive as determined by the maximal luminescence of antibody-attenuated hIL-2 fusion proteins in comparison to the maximal luminescence of rhIL-2. Antibody-attenuated hIL-2 fusion protein dose-titration curves that reached the maximal luminescence as the rhIL-2 were considered to be variants with full activity. Partial activity was calculated as a percentage of full activity using rhIL-2 maximal luminescence as 100%. Maximal RLU of antibody-attenuated hIL-2 fusion proteins with less than 10% of the rhIL-2 maximal RLU at the highest concentration of 1200 nM were considered to have no agonist activity or inactive. For some variants, EC₅₀ values were estimates only since a full curve was not reached. Many examples of anti-hPD-1-hIL-2 fusion proteins with attenuated hIL-2 showed rescued hIL-2 activity on the targeted cell line where the non-targeting antibody controls (denoted with 1H3) demonstrated no rescue of hIL-2 activity. Full rescue was illustrated by the reduction of fold-change from rhIL-2 to a value of 0) or 1.

TABLE 24
Fold change from rhIL-2 and agonistic activity of antibody-hIL-2
fusion proteins on TF1 + IL-2Rβ + hPD-1 cell
line (human PD-1 expressing cell line with intermediate-affinity IL-2R).

| | | Fold decrease | Agonistic |
|---|---|---|---|
| | | from rhIL-2 | Activity |
| | | (TF1+ IL-2Rβ + | (TF1 + IL-2Rβ + |
| | Variants | hPD-1) | hPD-1) |
| Non-Targeted | 1H3-hIgG4-df-hIL-2 (WT) | 1 | Full |
| Antibody- | 1H3-hIgG4-L6-hIL-2 (WT) | 1 | Full |
| Attenuated | 1H3-hIgG4-L6-hIL-2 (D20Y) | NC ^{a} | Inactive |
| hIL-2 | 1H3-hIgG4-df-hIL-2 (D20Y) | NC ^{a} | Inactive |
| Fusion | 1H3-hIgG4-L6-hIL-2 (D20A/R38P) | 3074 ^{a} | Partial, 80% |
| Proteins | 1H3-hIgG4-L6-hIL-2 (D20A/R38S) | 4482 ^{a} | Partial, 80% |
| | 1H3-hIgG4-L6-hIL-2 (D20A/R38D) | 2964 ^{a} | Partial, 60% |
| | 1H3-hIgG4-L6-hIL-2 | 3538 ^{a} | Partial, 80% |
| | (D20A/R38Q/E95A) | | |
| | 1H3-hIgG4-L6-hIL-2 | 657 ^{a} | Partial, 70% |
| | (D20A/F42H/E95A) | | |
| | 1H3-hIgG4-L6-hIL-2 (R38D/I92D) | 1428 ^{a} | Full |
| | 1H3-hIgG4-L6-hIL-2 (R38E/I92D) | 1887 ^{a} | Full |
| | 1H3-hIgG4-L6-hIL-2 (F42H/I92D) | 2024 ^{a} | Full |
| | 1H3-hIgG4-df-hIL-2 (D20A/R38E) | 307-3628 ^{a} | Partial, 70%-Full |
| | 1H3-hIgG4-L6-hIL-2 (D20A/R38E) | 4883-5226 | Partial, 70%-Full |
| | 1H3-hIgG4-L6-hIL-2 | 9167 ^{a} | Partial, 80% |
| | (T3A/D20A/R38E) | | |
| | 1H3-hIgG4-L6-hIL-2 | 4714 | Partial, 60% |
| | (D20A/R38E/C125A) | | |
| | 1H3-hIgG4-L6-hIL-2 | 4626 | Partial, 60% |
| | (T3A/D20A/R38E/C125A) | | |
| | 1H3-hIgG1-L6-hIL-2 (D20A/R38E) | 297 | Full |
| | 1H3-hIgG1-L6-hIL-2 | 513 | Partial, 80% |
| | (T3A/D20A/R38E) | | |
| | 1H3-hIgG1-L6-hIL-2 | 3342 | Partial, 80% |
| | (D20A/R38E/C125A) | | |
| | 1H3-hIgG1-L6-hIL-2 | 1081 | Partial, 90% |
| | (T3A/D20A/R38E/C125A) | | |
| | 1H3-hIgG1-LAGA-df-hIL-2 | 6459 | Full |
| | (T3A/D20A/R38E/C125A) | | |
| | 1H3-hIgG1-L6-hIL-2 (H16A) | 4 ^{a} | Full |
| | 1H3-hIgG1-L6-hIL-2 (F42A) | 0 ^{a} | Full |
| | 1H3-hIgG1-L6-hIL-2 (H16A/F42A) | 2 ^{a} | Full |
| | 1H3-hIgG1-L6-hIL-2 (D20T) | 75 ^{a} | Full |
| | 1H3-hIgG1-L6-hIL-2 | 2 ^{a} | Full |
| | (T3A/F42A/Y45A/L72G/C125A) | | |
| Anti-hPD-1- | A2-hIgG4-df-hIL-2 (D20A/R38E) | 0-1 ^{a} | Full |
| Attenuated | D12-hIgG4-df-hIL-2 (D20A/R38E) | 1 ^{a} | Full |
| hIL-2 | G12-hIgG4-df-hIL-2 (D20A/R38E) | 1 ^{a} | Full |
| Fusion | OMC476pB11-hIgG4-df-hIL-2 | 0 ^{a} | Full |
| Proteins | (D20A/R38E) | | |
| | OMC476pE4-hIgG4-df-hIL-2 | 2 ^{a} | Full |
| | (D20A/R38E) | | |
| | OMC476pG10-hIgG4-df-hIL-2 | 0 ^{a} | Full |
| | (D20A/R38E) | | |
| | OMC476pH10-hIgG4-df-hIL-2 | 1 ^{a} | Full |
| | (D20A/R38E) | | |
| | A2-hIgG4-df-hIL-2 (D20A/F42A) | 2 | Full |
| | A2-hIgG4-df-hIL-2 (D20A/F42S) | 1 ^{a} | Full |
| | A2-hIgG4-df-hIL-2 (D20S/R38E) | 0 ^{a} | Full |
| | A2-hIgG4-df-hIL-2 (F42A/N88R) | 0 ^{a} | Full |
| | A2-hIgG4-df-hIL-2 (F42I/I92D) | 9 ^{a} | Full |
| | A2-hIgG4-df-hIL-2 (F42Q/I92D) | 5 ^{a} | Full |
| | A2-hIgG4-df-hIL-2 (F42T/I92D) | 1 ^{a} | Full |
| | A2-hIgG4-df-hIL-2 (F42W/I92D) | 4 ^{a} | Full |
| | A2-hIgG4-df-hIL-2 (R38E/D84K) | 0-1 ^{a} | Full |
| | A2-hIgG4-df-hIL-2 (R38E/I92K) | 0-1 ^{a} | Full |
| | C51E6-5-hIgG4-df-hIL-2 | 0-1 ^{a} | Partial, |
| | (D20A/R38E) | | 70%-Full |
| | C51E6-5-hIgG4-L6-hIL-2 | 1 ^{a} | Partial, 90% |
| | (D20A/R38E) | | |
| | C51E6-5-hIgG4-LE-df-hIL-2 | 1 ^{a} | Full |
| | (T3A/D20A/R38E/C125A) | | |
| | C51E6-5-hIgG4-LAGA-df-hIL-2 | 0 ^{a} | Full |
| | (T3A/D20A/R38E/C125A) | | |
| | OMC.1.B6-hIgG4-L6-hIL-2 | 0 ^{a} | Partial, 60% |
| | (D20A/R38E) | | |
| | OMC.1.D6-hIgG4-L6-hIL-2 | 0 ^{a} | Partial, 90% |
| | (D20A/R38E) | | |
| | OMC.2.C6-hIgG4-L6-hIL-2 | 0 | Partial, 60% |
| | (D20A/R38E) | | |
| | 2A3.H7-hIgG4-df-hIL-2 | 0 | Full |
| | (D20A/R38E) | | |
| | 1H9-hIgG4-df-hIL-2 (D20A/R38E) | 0 ^{a} | Full |
| | 1D5-hIgG4-df-hIL-2 (D20A/R38E) | 1 ^{a} | Full |
| | 2H7-hIgG1-df-hIL-2 | 1 ^{a} | Full |
| | (T3A/D20A/R38E/C125A) | | |
| | 2H7-hIgG1-LE-df-hIL-2 | 0 ^{a} | Full |
| | (T3A/D20A/R38E/C125A) | | |
| | 2H7-hIgG1-LAGA-df-hIL-2 | 1 ^{a} | Full |
| | (T3A/D20A/R38E/C125A) | | |
| | 2H7-hIgG4-df-hIL-2 | 1 ^{a} | Full |
| | (T3A/D20A/R38E/C125A) | | |
| | 2H7-hIgG4-LE-df-hIL-2 | 1-4 ^{a} | Full |
| | (T3A/D20A/R38E/C125A) | | |
| | 2H7-hIgG4-LAGA-df-hIL-2 | 1 ^{a} | Full |
| | (T3A/D20A/R38E/C125A) | | |
| | H7-767 | 0-1 ^{a} | Full |
| | 2H7-hIgG1-df-hIL-2 | 2 ^{a} | Full |
| | (T3A/R38E/D84K/C125A) | | |
| | 2H7-hIgG1-LE-df-hIL-2 | 1 ^{a} | Full |
| | (T3A/R38E/D84K/C125A) | | |
| | 2H7-hIgG1-LAGA-df-hIL-2 | 1 ^{a} | Full |
| | (T3A/R38E/D84K/C125A) | | |
| | 2H7-hIgG4-df-hIL-2 | 1 ^{a} | Full |
| | (T3A/R38E/D84K/C125A) | | |
| | 2H7-hIgG4-LE-df-hIL-2 | 0-2 ^{a} | Full |
| | (T3A/R38E/D84K/C125A) | | |
| | 2H7-hIgG4-LAGA-df-hIL-2 | 1-3 ^{a} | Full |
| | (T3A/R38E/D84K/C125A) | | |
| | 2H7-hIgG1-df-hIL-2 | 1 ^{a} | Full |
| | (T3A/R38E/I92K/C125A) | | |
| | 2H7-hIgG1-LE-df-hIL-2 | 1 ^{a} | Full |
| | (T3A/R38E/I92K/C125A) | | |
| | 2H7-hIgG1-LAGA-df-hIL-2 | 1-2 ^{a} | Full |
| | (T3A/R38E/I92K/C125A) | | |
| | 2H7-hIgG4-df-hIL-2 | 1 ^{a} | Full |
| | (T3A/R38E/I92K/C125A) | | |
| | 2H7-hIgG4-LE-df-hIL-2 | 1 ^{a} | Full |
| | (T3A/R38E/I92K/C125A) | | |
| | 2H7-hIgG4-LAGA-df-hIL-2 | 1 ^{a} | Full |
| | (T3A/R38E/I92K/C125A) | | |
| | 2H7-hIgG1-LAGA-df-hIL2 | Not Attenuated | Full |
| | (T3A/D20S/R38E/C125A) | on graph; NC ^{a} | |
| | 2H7-hIgG1-LAGA-df-hIL2 | 0 | Full |
| | (T3A/R38E/D84F/C125A) | | |
| | 2H7-hIgG1-LAGA-df-hIL2 | 0 ^{a} | Full |
| | (T3A/R38E/I92R/C125A) | | |
| | 2H7-hIgG1-LAGA-df-hIL2 | 2 ^{a} | Full |
| | (T3A/R38E/I92E/C125A) | | |
| | 2H7-hIgG1-LAGA-df-hIL2 | Not Attenuated | Full |
| | (T3A/R38E/I92S/C125A) | on graph; NC ^{a} | |
| | 2H7-hIgG1-LAGA-df-hIL2 | 0 ^{a} | Full |
| | (T3A/R38E/I92D/C125A) | | |
| | 2H7-hIgG1-LAGA-df-hIL2 | >10,000 ^{a} | Full |
| | (T3A/H16E/R38E/C125A) | | |

NC = Not Calculated by GraphPad Prism 7
^{a} = Fold change is an estimate only since a full four parameter logistic curve was not reached

### Example 18: Evaluation of Surrogate Anti-hPD-1-Attenuated hIL-2 Fusion Proteins that Block or do not Block Mouse PD-L1 in an In Vivo Murine Colon Adenocarcinoma (MC38) Model

Since there are no accepted models to explore in vivo efficacy of oncology therapeutics in primates, a surrogate anti-mPD-1-attenuated hIL-2 fusion protein was generated and tested in a syngeneic murine tumor model. This MC38 colon adenocarcinoma model is routinely used to test efficacy of immuno-oncology therapeutics. To explore the in vivo effect of the anti-PD-1-attenuated hIL-2 fusion protein, a surrogate anti-mouse PD-1 antibody designated RMP1-14 (known to block mouse PD-L1 binding) and RMP1-30 (described as a mouse PD-L1 non-blocker) was fused to an attenuated hIL-2 at the C-terminus of the mouse IgG2b-N297A heavy chain and tested in an MC38 colon adenocarcinoma model. The hIL-2 moiety included the substitutions F42K. Y45R. and V69R that were tested on an IL-2 dependent mouse T lymphoblast cell line (CTLL-2) and that were demonstrated to be attenuated for mouse IL-2 activity. Human IL-2 can stimulate proliferation of mouse T cells at similar concentrations, however the same substitutions that attenuate activity on human IL-2 dependent cell lines do not attenuate activity on the CTLL-2 cell line (data not shown). As such, the F42K/Y45R/V69R substitutions were used in hIL-2 as a surrogate since they demonstrated attenuated IL-2 activity on mouse cell lines. Sequences comprising the heavy and light chain variable region sequences of anti-mouse PD-1 antibodies RMP1-14 and RMP1-30 (as described in Matsumoto K et al., J Immunol. 2004 Feb. 15; 172 (4): 2530-41) were also formatted onto a murine IgG2b-N297A background to generate anti-mPD-1 RMP1-14 mIgG2b-N297A (SEQ ID NOs: 564 and 566) and anti-mPD-1 RMP1-30 mIgG2b-N297A (SEQ ID NOs: 567 and 568). The mouse IgG2b isotype with an N297A substitution is the murine equivalent of an Fc isotype that abrogates Fc immune effector function. Surrogate antibodies and antibody-attenuated hIL-2 fusion proteins were produced, expressed and Protein-A purified using standard techniques.

In this murine tumor model. ten week old female C57BL/6NCrl (Charles River) mice were injected into the right flank with 5×10⁵ MC38 colorectal carcinoma cells. When tumors reached 80-120 mm³, mice were sorted into cohorts (10 mice/group) and treatment began on day 1 of study. Anti-mPD-1 RMP1-14 mIgG2b-N297A. anti-mPD-1 RMP1-30 mIgG2b-N297A, anti-mPD-1 RMP1-14 mIgG2b-N297A-L6-hIL-2 (F42K/Y45R/V69R) (SEQ ID NOs: 565 and 566), and anti-mPD-1 RMP1-30 mIgG2b-N297A-L6-hIL-2 (F42K/Y45R/V69R) (SEQ ID NOs: 568 and 569) were dosed intraperitoneally at 5 mg/kg twice weekly for 4 weeks along with vehicle control (phosphate-buffered saline). Tumor size was measured with calipers twice weekly using the formula (w2×L)/2 where w=width and L=length for the duration of the study. The study endpoint was a tumor volume of 1000 mm³ or survival at day 50, whichever came first.

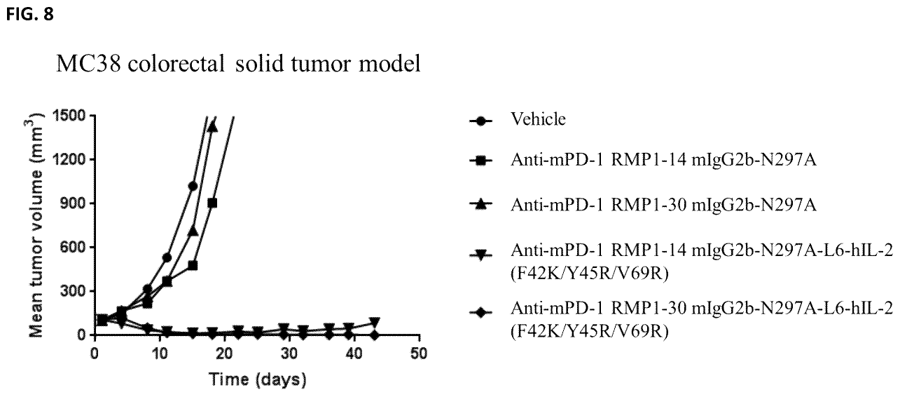

FIG. 8 demonstrates that although the administration of anti-mPD-1 RMP1-14-mIgG2b-N297A or anti-mPD-1 RMP1-30-mIgG2b-N297A antibodies alone did not promote significant efficacy relative to treatment with vehicle control, the administration of anti-mPD-1 RMP1-14 mIgG2b-N297A-L6-hIL-2 (F42K/Y45R/V69R) or anti-mPD-1 RMP1-30 mIgG2b-N297A-L6-hIL-2 (F42K/Y45R/V69R) anti-PD-1-attenuated hIL-2 fusion proteins was associated with 90% and 100% complete tumor regressions respectively. These data demonstrate that the anti-tumor efficacy mediated by anti-mPD-1-hIL-2 (F42K/Y45R/V69R) fusion proteins does not require PD-1 checkpoint blockade and that efficacy is dependent on hIL-2 activity. The data further demonstrate that antibody mediated targeting of PD-1 expressing T cells is sufficient to promote potent anti-tumor efficacy in the MC38 tumor model.

### Example 19: Surrogate Anti-hPD-1-Attenuated hIL-2 Fusion Protein Expands Effector Memory CD8+ T Cells in an In Vivo Murine Colon Adenocarcinoma Model

To understand the mechanism-of-action of the surrogate anti-hPD-1-attenuated hIL-2 fusion protein in vivo, a similar in vivo experiment to Example 18 was performed, followed by immunophenotyping of the resultant T cell populations in tumors, blood, spleens and lymph nodes after three doses. Ten week old female C57BL/6NCrl (Charles River) mice were subcutaneously implanted with the 5×10⁵ murine MC38 colon adenocarcinoma cancer tumor cells into the right flank and tumors were monitored for growth. Animals with tumors between 150-260 mm³ were divided between four groups with 10 mice per group for the study. After 21 days post-implantation, animals were dosed intraperitoneally with 0.2 mL/dose phosphate buffered saline (PBS) for the vehicle control. 5 mg/kg anti-KLH-C3-mIgG2b-N297A-L6-hIL-2 (F42K/Y45R/V69R), 5 mg/kg anti-mPD-1 RMP1-30 mIgG2b-N297A. or 5 mg/kg anti-mPD-1 RMP1-30 mIgG2b-N297A-L6-hIL-2 (F42K/Y45R/V69R) on days 1, 4 and 8. On day 9, tumors, spleens and inguinal lymph nodes were harvested from all mice and processed into single cell suspensions for subsequent flow cytometry analysis.

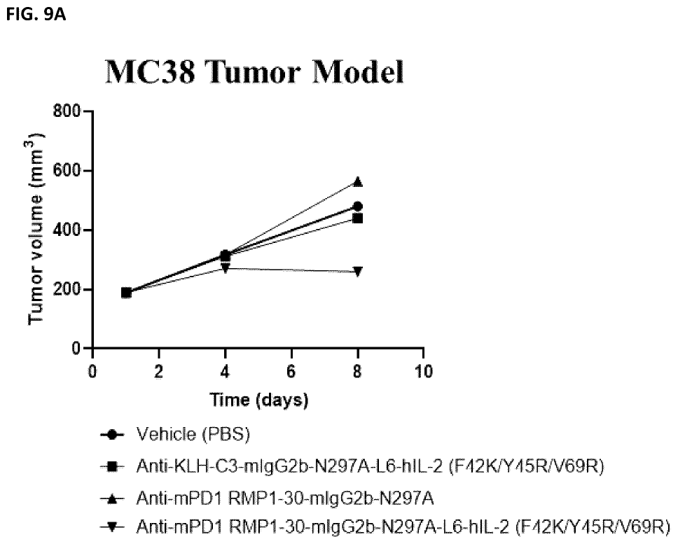

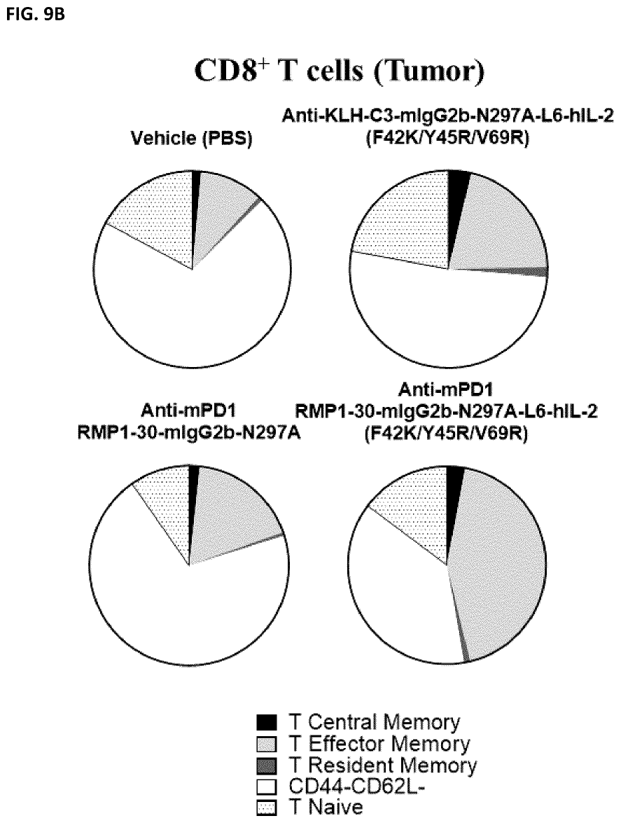

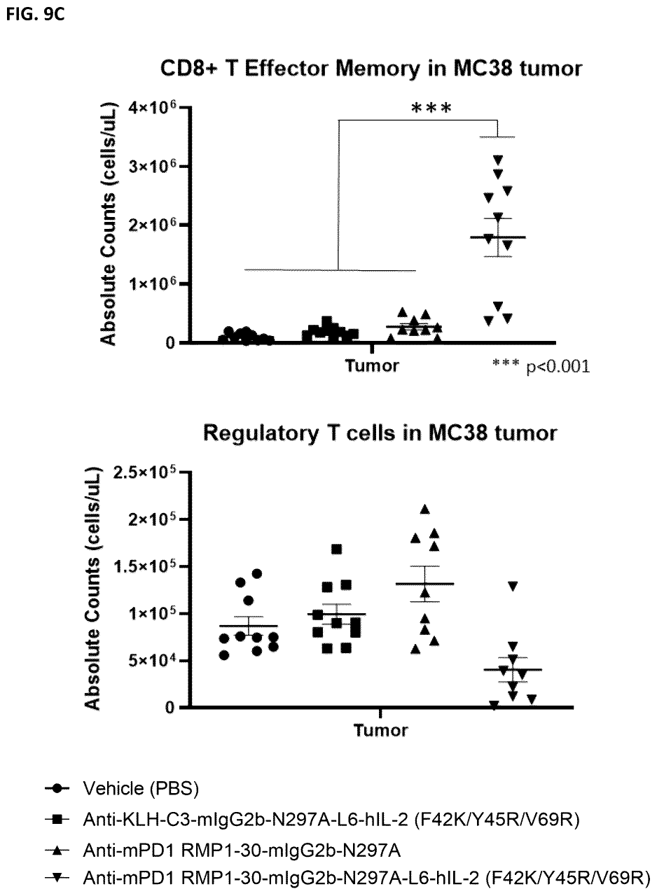

FIG. 9A charts the tumor volume growth (mm³) over 9 days from the first dose on day 1 where each point represents a mean of 10 mice. By day 8, anti-mPD-1 RMP1-30 mIgG2b-N297A-L6-hIL-2 (F42K/Y45R/V69R) had a reduction in tumor volume compared to other treatment groups. FIG. 9B summarizes the contribution of various CD8⁺ T cell subsets in the tumor of each treatment group in which T Central Memory were phenotyped as CD45⁺CD3⁺CD4⁺CD8⁺CD44⁺CD127⁺CD69⁻CD103⁻. T Effector Memory were CD45⁺CD3⁺CD4⁺CD8⁺CD44⁺CD127⁺CD69⁺CD103⁺CD62L⁻. T Resident Memory were CD45⁺CD3⁺CD4⁺CD8⁺CD44⁺CD127⁺CD69⁺CD103, CD44⁺CD62L⁻ T cells were CD45⁺CD3⁺CD4⁺CD8⁺CD44⁺CD62L⁻ and T Naïve were CD45⁺CD3⁺CD4⁺CD8⁺CD44⁻CD62L⁺. In comparison to other treatment groups, there was expansion of the CD8⁺ T Effector Memory subset in the anti-mPD-1 RMP1-30 mIgG2b-N297A-L6-hIL-2 (F42K/Y45R/V69R) treated mice as indicated in the increase of the light grey slice of FIG. 9B. This was also illustrated in FIG. 9C in absolute counts (cells/μL) within the MC38 dissected tumor. Furthermore, within the tumor, there was a decrease in the absolute counts (cells/μL) of Regulatory T cells defined as expressing CD45 CD3 CD4 CD8 CD25 FoxP3 markers.

The expansion of CD8⁺ T Effector Memory and decrease in Regulatory T cells has been associated with effective immunotherapy in both mice and humans.

### Example 20: Anti-hPD-1-Attenuated hIL-2 Fusion Proteins are Active In Vivo in an NCG-PBMC Model

Engrafting human immune cells into NOD-Prkdc^{em26Cd52}IL-2rg^{em26Cd22}/NjuCrl (NCG) mice that lack functional T, B, and NK cells has been a valuable tool for evaluating efficacy of therapeutics hypothesized to stimulate human T cells. In this model, if the therapeutic activates human T cells, there would be a resulting expansion of T cells and accelerated graft-versus-host disease (GvHD).

Three independent donors for human peripheral mononuclear cell (hPBMC) engraftment were evaluated over a 4 week period for engraftment kinetics as well as expression of human PD-1 and human IL-2 receptors on T cells. Of the three donors tested, the donor that induced the most T cells with an intermediate window for GvHD was chosen. 1.5×10⁷ hPBMCs were intravenously injected into NCG mice and divided into 8 groups of 8-16 mice. On days 7, 10, and 14, mice were intraperitoneally injected with three doses of 2H7-hIgG1-LAGA-df-hIL-2 (T3A/D20A/R38E/C125A) (SEQ ID NOs: 471, 425) (2.5 mg/kg, 5 mg/kg, or 10 mg/kg), 1H3-hIgG1-LAGA-df-hIL-2 (T3A/D20A/R38E/C125A) (SEQ ID NOs: 546, 374) (5 mg/kg or 10 mg/kg), 1H3-hIgG1-LAGA-df-hIL-2 (T3A/C125A) (SEQ ID NOs: 563, 374) (10 mg/kg), or 2H7-hIgG1-LAGA-df-hIL-2 (T3A/R38E/192K/C125A) (SEQ ID NOs: 474, 425) (5 mg/kg). The anti-DNase fusion protein both as a wild-type hIL-2 (1H3-hIgG1-LAGA-df-hIL-2 (T3A/C125A)) and with the attenuated hIL-2 moiety (1H3-hIgG1-LAGA-df-hIL-2 (T3A/D20A/R38E/C125A)) was used as a non-targeting antibody control. Although the 1H3-hIgG1-LAGA-df-hIL-2 (T3A/C125A) fusion protein had no changes in the hIL-2 moiety which reduce hIL-2 activity, it did comprise the T3A and C125A substitutions to remove the predicted O-linked glycosylation site on human IL-2 (see for example Int'l Pub. No. WO2012/107417) and unpaired cysteine residue (see for example Int'l Pub. No. WO2018/184964), respectively. These substitutions have not demonstrated reduced hIL-2 potency in the clinic. On Day 21, blood, spleen, and lungs were harvested in which blood and spleens were processed for flow cytometry immunophenotyping while lungs were weighed.

After 21 days, flow cytometry immunophenotyping was performed on the blood and spleens of animals. Table 25 summarizes the markers used to delineate human T cell populations for subsequent analysis.

TABLE 25
Phenotypic markers to define human
T cell subsets in NCG-PBMC mice

| Cell Population | Phenotypic Markers |
|---|---|
| Pan T cells | CD3+ |
| CD8+ Naive | CD3+CD4−CD8+CD45RO−CCR7+ |
| CD8+ Effector | CD3+CD4−CD8+CD45RO−CCR7− |
| CD8+ Effector Memory | CD3+CD4−CD8+CD45RO+CCR7− |
| CD8+ Central Memory | CD3+CD4−CD8+CD45RO+CCR7+ |
| CD4+ Naïve | CD3+CD4+CD8−CD45RO−CCR7+ |
| CD4+ Effector | CD3+CD4+CD8−CD45RO−CCR7− |
| CD4+ Effector Memory | CD3+CD4+CD8−CD45RO+CCR7− |
| CD4+ Central Memory | CD3+CD4+CD8−CD45RO+CCR7+ |
| Regulatory T cells | CD3+CD4+CD8−CD25+Foxp3+ |
| NK Cells | CD3−CD56+ |

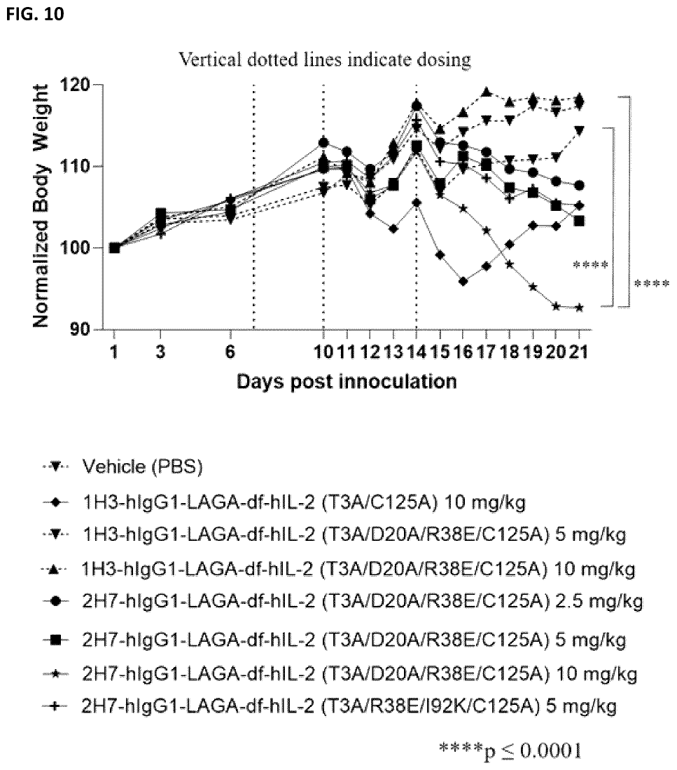

Body weight was measured for 21 days and normalized to day 1 for each individual animal as an assessment of graft-versus-host disease (GvHD) as illustrated in FIG. 10. Accelerated GvHD was observed in the 2H7-hIgG1-LAGA-df-hIL-2 (T3A/D20A/R38E/C125A) treated mice at 10 mg/kg. A small decrease in body weight was also observed in the 2H7-hIgG1-LAGA-df-hIL-2 (T3A/D20A/R38E/C125A) treated mice at 2.5 mg/kg, 5 mg/kg, and 2H7-hIgG1-LAGA-df-hIL-2 (T3A/R38E/192K/C125A) at 5 mg/kg. Although body weight loss was seen in the 1H3-hIgG1-LAGA-df-hIL-2 (T3A/C125A), it was not sustained.

The flow cytometry analysis correlated with the accelerated graft-versus-host disease (GvHD) observed. Using the phenotypic markers for human T cell subset delineation provided in Table 25, flow cytometry analysis of peripheral blood demonstrated only a minor expansion of CD3⁺, CD4⁺, and CD8⁺ T cell subsets (as quantified by a fold change from vehicle control of between 10-fold to 50-fold for CD3+ T cells) in mice treated with 2H7-hIgG1-LAGA-df-hIL-2 (T3A/D20A/R38E/C125A) at 2.5 mg/kg and 5 mg/kg, and mice treated with 1H3-hIgG1-LAGA-df-hIL-2 (T3A/C125A) at 10 mg/kg. Furthermore, CD3⁺, CD4⁺, and CD8⁺ T cell subsets were greatly expanded (fold change from vehicle control was greater than 50-fold for CD3⁺ T cells) in the peripheral blood of mice treated with 2H7-hIgG1-LAGA-df-hIL-2 (T3A/D20A/R38E/C125A) at 10 mg/kg. Table 26 summarizes the expanded human T cell subsets.

TABLE 26
Expansion of human CD3⁺, CD4⁺, and CD8⁺ T cell subsets in NCG-PBMC mice

| | Fold Change in numbers (Blood) | | |
|---|---|---|---|
| Agent | CD3⁺ T cells | CD4⁺ T cells | CD8⁺ T cells |
| Vehicle (PBS) | 1 | 1 | 1 |
| 1H3-hIgG1-LAGA-df-hIL-2 (T3A/C125A) 10 mg/kg | 22.76 | 27.73 | 16.13 |
| 1H3-hIgG1-LAGA-df-hIL-2 (T3A/D20A/R38E/C125A) 5 mg/kg | 1.94 | 2.12 | 1.74 |
| 1H3-hIgG1-LAGA-df-hIL-2 (T3A/D20A/R38E/C125A) 10 mg/kg | 0.56 | 0.56 | 0.57 |
| 2H7-hIgG1-LAGA-df-hIL-2 (T3A/D20A/R38E/C125A) 2.5 mg/kg | 24.57 | 32.4 | 14.4 |
| 2H7-hIgG1-LAGA-df-hIL-2 (T3A/D20A/R38E/C125A) 5 mg/kg | 53.03 | 70.74 | 22.86 |
| 2H7-hIgG1-LAGA-df-hIL-2(T3A/D20A/R38E/C125A) 10 mg/kg | 203.3 | 296.94 | 58.82 |
| 2H7-hIgG1-LAGA-df-hIL-2 (T3A/R38E/I92K/C125A) 5 mg/kg | 31.79 | 48.76 | 8.79 |

N/A = Not Applicable

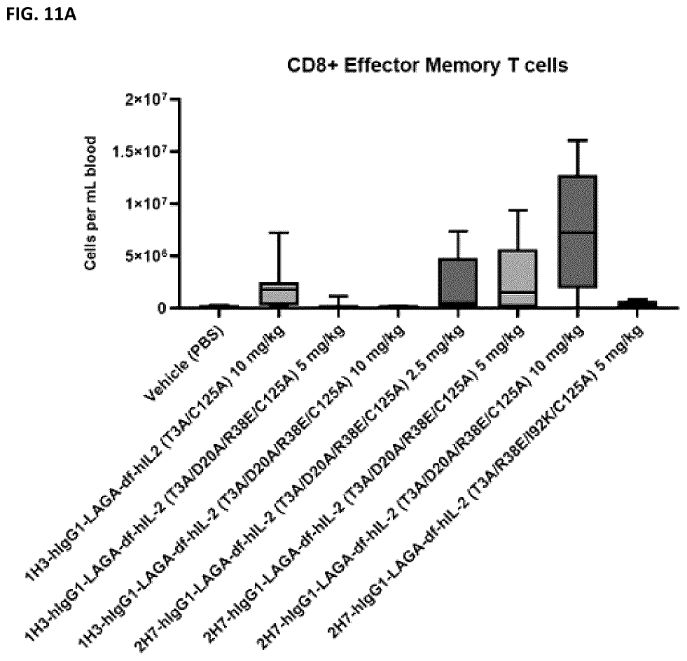

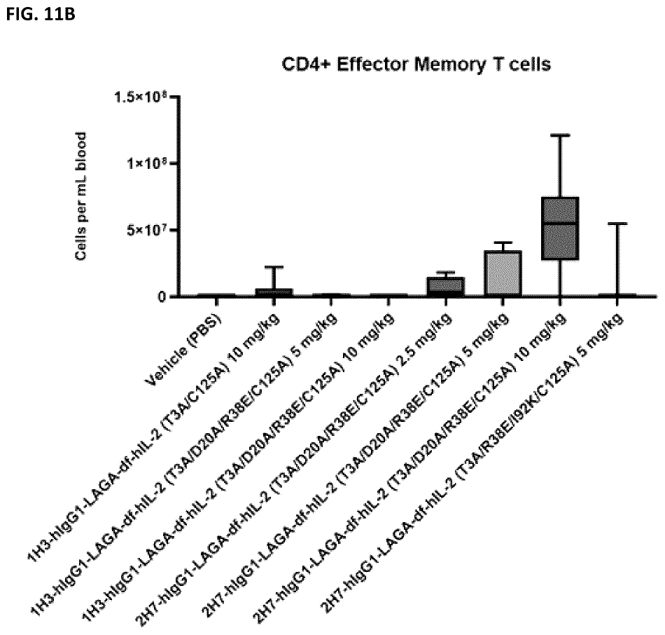

In addition to evaluating CD3⁺, CD4⁺, and CD8⁺ T cells between treatment groups, the memory and naïve subsets for CD4⁺ and CD8⁺ T cell subsets were also assessed. The phenotypic markers used for delineation of Naïve, Effector, Effector Memory and Central Memory for both CD4⁺ and CD8⁺ T cell is summarized in Table 25. There were no changes in Naïve, Effector or Central Memory T cells between treatment groups (data not shown). However, mice treated with 2H7-hIgG1-LAGA-df-hIL-2 (T3A/D20A/R38E/C125A) at 10 mg/kg had greatly expanded CD4⁺ and CD8⁺ Effector Memory (EM) T cells in the peripheral blood with an average cell number per milliliter greater than 5 million for CD8⁺ T cells and greater than 50 million for CD4⁺ T cells (FIGS. 11A and 11B). Box-and-whisker plots were graphed with the box around the first and third quartile, the horizontal line as the median, and lines indicated the minimum and maximum points. There was moderate expansion of CD8⁺ Effector Memory (EM) T cells defined as an average cell number per million between 1 to 5 million per milliliter for animals treated with 2H7-hIgG1-LAGA-df-hIL-2 (T3A/D20A/R38E/C125A) at 2.5 mg/kg and 5 mg/kg as well as for 1H3-hIgG1-LAGA-df-hIL-2 (T3A/C125A). There was moderate expansion of CD4⁺ Effector Memory (EM) T cells between 6 to 13 million per milliliter for CD4⁺ T cells in the mice treated with 2H7-hIgG1-LAGA-df-hIL-2 (T3A/D20A/R38E/C125A) at 2.5 mg/kg and 5 mg/kg.

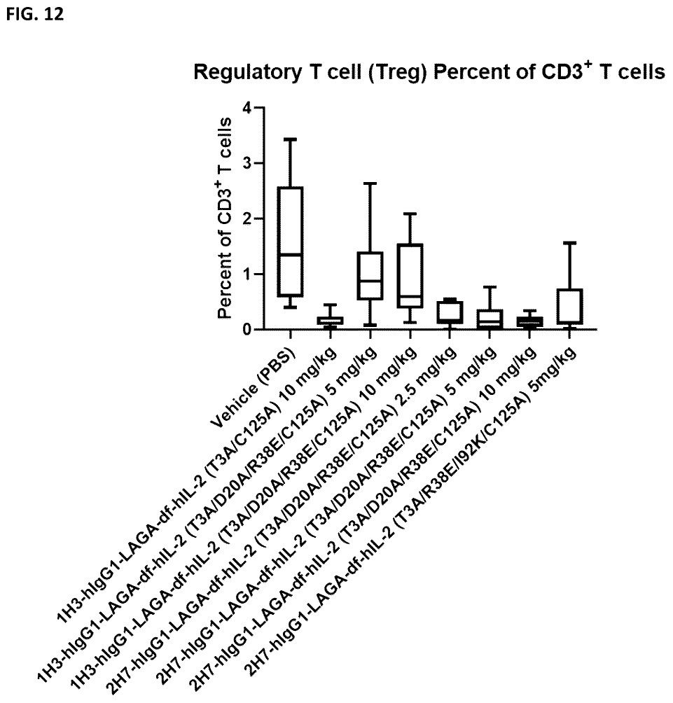

In addition to stimulating effector T cells, IL-2 has been described to stimulate NK cells and regulatory T cells (Tregs) and since Tregs express high levels of CD25 and NK cells express CD122, these immune cell types were also evaluated. FIG. 12 illustrates that animals treated with 2H7-hIgG1-LAGA-df-hIL-2 (T3A/D20A/R38E/C125A) at the highest dose of 10 mg/kg did not expand human regulatory T cells and instead had the lowest percent of regulatory T cells (as phenotypically defined in Table 25) in the peripheral blood of animals. There was a dose-dependent decrease of human regulatory T cells and in comparison to vehicle control that had an average of 1.6% human CD3⁺ T cells that were Tregs, 2H7-hIgG1-LAGA-df-hIL-2 (T3A/D20A/R38E/C125A) at 10 mg/kg had an average of 0.16% human CD3⁺ T cells that were Tregs. There were no changes in the percentage of human NK cells in peripheral blood (phenotype defined in Table 25) in all treatment groups in comparison to vehicle control (data not shown).

### Example 21: Non-Clinical Safety Profile of Anti-hPD-1-Attenuated hIL-2 Fusion Proteins

Cynomolgus monkeys previously have been used to evaluate the toxicity of unmodified IL-2. Lethality was observed in cynomolgus monkeys at exogenous recombinant IL-2 doses as low as 50 μg/kg/day. Since the binding of H7-767 to cynomolgus monkey hPD-1 on primary activated PBMCs was confirmed by flow cytometry (Example 12), a single-dose study for preliminary safety assessment was performed with both a variant of H7-767 (H7-02-hIgG1-LAGA-df-hIL-2 (T3A/D20A/R38E/C125A) (SEQ ID NOs: 582 and 583) and H7-767. H7-02-hIgG1-LAGA-df-hIL-2 (T3A/D20A/R38E/C125A) was delivered by 15 minute iv infusion to 8 monkeys at 1 mg/kg (4 animals) or 10 mg/kg (4 animals). Sampling at time-points up to 360 hours following infusion was performed. No adverse effects, gross toxicities, body weight loss, or lethality was observed (data not shown). A follow-up single-dose study using H7-767 was performed at higher doses of 5 mg/kg and 50 mg/kg similar to the first study, with sampling at time-points up to 360 hours post-infusion. Again, no adverse effects, gross toxicities, body weight loss or lethality was observed (data not shown).

### Example 22: Attenuation of IL-2 Activity of Modified hIL-2 Proteins

The attenuation of IL-2 activity of modified hIL-2 proteins comprising a substitution at amino acid position 20 (D20) and a substitution at amino acid position 38 (R38) was tested in proliferation assays in both the NK-92 and TF1+IL-2Rβ cell lines as described in Example 5 above. The modified hIL-2 proteins were grouped into 7 groups (1 to 7) based upon the maximal agonist activity of the modified hIL-2 protein and the level of attenuation of potency on both the intermediate and high-affinity receptors (Table 27) relative to non-modified recombinant hIL-2. The criteria used for grouping the modified hIL-2 proteins was:
Group 1: Variants with the highest attenuation (i.e., >10,000-fold) and at least about 80% activity on the intermediate-affinity receptor but also had high attenuation and at least about 70% activity on the high-affinity receptor.
Group 2: Variants with at least about 70% activity and >1,000-fold attenuation on the intermediate-affinity receptor, and about 20% activity to about 30% activity on the high-affinity receptor.
Group 3: Variants with about 50% activity to about 70% activity and >1,000-fold attenuation on the intermediate-affinity receptor, and about 20% activity on the high-affinity receptor.
Group 4: Variants with at least about 70% activity but only >500-fold attenuation on the intermediate-affinity receptor, and about 50% activity on the high-affinity receptor.
Group 5: Variants with at least about 70% activity on both receptors but >10-fold to >300-fold attenuation on the intermediate-affinity receptor in descending order and 70-fold to 1500-fold attenuation on the high-affinity receptor also in descending order.
Group 6: Variants with only about 30% activity and >2,500-fold attenuation on the intermediate-affinity receptor, and no activity on the high-affinity receptor.
Group 7: Variants with no activity on both the intermediate-affinity receptor and high-affinity receptor.

TABLE 27
Fold change from rhIL-2 and agonistic activity of modified hIL-2 proteins
comprising a substitution at amino acid position 20 (D20) and a substitution
at amino acid position 38 (R38) in a cell-based proliferation assay

| | | SEQ ID NO | Fold change | Agonistic | Fold change | Agonistic |
|---|---|---|---|---|---|---|
| | | of hIL-2 | from rhIL-2 | Activity | from rhIL-2 | Activity |
| | Variants | variant | (NK-92) | (NK-92) | (TF1 + IL-2Rβ) | (TF1 + IL-2Rβ) |
| Group | 1H3-hIgG1- | 149 | 1183-2016 | at least | >10,000 ^{a} | at least |
| 1 | L6-hIL-2 | | | about 70% | | about 80% |
| | (D20A/R38E) | | | | | |
| Group | 1H3-hIgG1- | 608 | >10,000 ^{a} | about 30% | 6665 | at least |
| 2 | L6-hIL-2 | | | | | about 70% |
| | (D20Q/R38E) | | | | | |
| Group | 1H3-hIgG1- | 614 | >10,000 ^{a} | about 30% | 2607 | at least |
| 2 | L6-hIL-2 | | | | | about 70% |
| | (D20M/R38E) | | | | | |
| Group | 1H3-hIgG1- | 611 | >10,000 ^{a} | about 20% | 1782 | at least |
| 2 | L6-hIL-2 | | | | | about 70% |
| | (D20I/R38E) | | | | | |
| Group | 1H3-hIgG1- | 620 | >10,000 on | about 20% | 1849 | about 50% |
| 3 | L6-hIL-2 | | graph, NC ^{a} | | | |
| | (D20V/R38E) | | | | | |
| Group | 1H3-hIgG1- | 307 | >10,000 on | about 50% | 626 | at least |
| 4 | L6-hIL-2 | | graph, NC ^{a} | | | about 70% |
| | (D20S/R38E) | | | | | |
| Group | 1H3-hIgG1- | 607 | 1521 | at least | 378 | at least |
| 5 | L6-hIL-2 | | | about 70% | | about 70% |
| | (D20N/R38E) | | | | | |
| Group | 1H3-hIgG1- | 610 | 1288 | at least | 212 | at least |
| 5 | L6-hIL-2 | | | about 70% | | about 70% |
| | (D20G/R38E) | | | | | |
| Group | 1H3-hIgG1- | 617 | 524 | at least | 75 | at least |
| 5 | L6-hIL-2 | | | about 70% | | about 70% |
| | (D20T/R38E) | | | | | |
| Group | 1H3-hIgG1- | 609 | 77 | at least | 12 | at least |
| 5 | L6-hIL-2 | | | about 70% | | about 70% |
| | (D20E/R38E) | | | | | |
| Group | 1H3-hIgG1- | 306 | No activity | No activity | 2945 | about 30% |
| 6 | L6-hIL-2 | | | | | |
| | (D20H/R38E) | | | | | |
| Group | 1H3-hIgG1- | 612 | >10,000 ^{a} | No activity | >10,000 ^{a} | No activity |
| 7 | L6-hIL-2 | | | | | |
| | (D20L/R38E) | | | | | |
| Group | 1H3-hIgG1- | 613 | No activity | No activity | 544 | No activity |
| 7 | L6-hIL-2 | | | | | |
| | (D20K/R38E) | | | | | |
| Group | 1H3-hIgG1- | 615 | >10,000 ^{a} | No activity | >10,000 ^{a} | No activity |
| 7 | L6-hIL-2 | | | | | |
| | (D20F/R38E) | | | | | |
| Group | 1H3-hIgG1- | 616 | >10,000 ^{a} | No activity | >10,000 ^{a} | No activity |
| 7 | L6-hIL-2 | | | | | |
| | (D20P/R38E) | | | | | |
| Group | 1H3-hIgG1- | 618 | >10,000 ^{a} | No activity | >10,000 ^{a} | No activity |
| 7 | L6-hIL-2 | | | | | |
| | (D20W/R38E) | | | | | |
| Group | 1H3-hIgG1- | 619 | >10,000 ^{a} | No activity | 1 | No activity |
| 7 | L6-hIL-2 | | | | | |
| | (D20Y/R38E) | | | | | |
| Group | 1H3-hIgG1- | 606 | >10,000 ^{a} | No activity | >10,000 ^{a} | No activity |
| 7 | L6-hIL-2 | | | | | |
| | (D20R/R38E) | | | | | |

### Example 23: Activity of Surrogate Fusion Protein in a Murine MC38 Colo-Rectal Tumor Model

Ten week old female C57BL/6NCrl mice were injected into the right flank with 5×10⁵ syngeneic MC38 colorectal carcinoma cells. When tumors reached 80-120 mm³, mice were sorted into cohorts (10 mice/group) and treatment began on day 1 of study. All agents except hIL-2 were dosed intraperitoneally at 5 mg/kg twice weekly for 4 weeks, starting on day 1. hIL-2 was dosed intraperitoneally at 36,000 International Units once a day from days 1-5. Tumor size was measured with calipers twice weekly for the duration of the study. The study endpoint was a tumor volume of 1000 mm³ or survival at day 50 or progression free survival at day 70, whichever came first.

All test agents including antibody molecules and antibody-hIL-2 fusion proteins were generated using a mouse IgG2b Fc region with a single N297A amino-acid substitution at position 297, which prevents glycosylation of the Fc region and significantly reduces any Fc region-mediated immune effector function, thereby preventing cellular depletion in vivo. Anti-mPD-1 RMP1-14 is a monoclonal antibody antagonist of the mouse PD-1 receptor (Matsumoto, J Immunol 172:2530-2541, 2004). Anti-mPD-1 RMP1-14-hIL-2 F42K/Y45R/V69R is a bi-functional fusion protein consisting of the monoclonal RMP1-14 antibody antagonist of the mouse PD-1 receptor fused at its C-terminus via a flexible six amino-acid glycine/serine linker to hIL-2 F42K/Y45R/V69R (SEQ ID NO: 621) that is a reduced potency IL-2 variant. This molecule was designed to target a reduced potency hIL-2 variant directly to PD-1 expressing T cells in vivo in mice. Anti-KLH-hIL-2 F42K, Y45R, V69R is a control fusion protein consisting of an isotype control monoclonal antibody recognizing a non-mammalian antigen (keyhole limpet hemocyanin, KLH) fused at its C-terminus via a flexible six amino-acid glycine/serine linker to hIL-2 F42K, Y45R, V69R that is a reduced potency IL-2 variant.

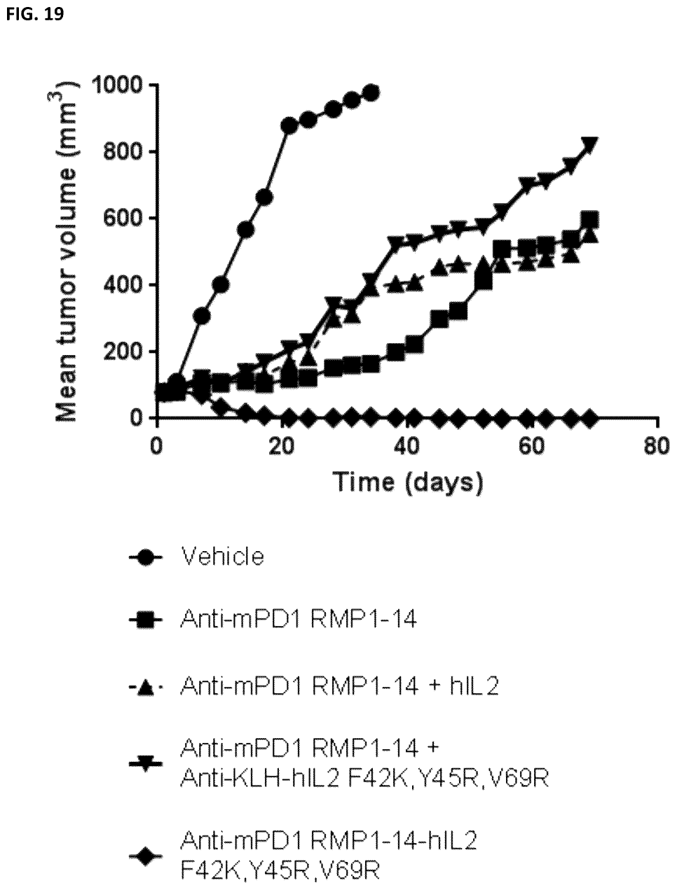

Results are presented in FIG. 19. The MC38 colo-rectal tumor model is particularly responsive to antibody mediated PD-1 receptor inhibition. Although tumors growing in vehicle-treated mice rapidly reached study endpoint, 50% of mice treated with anti-mPD-1 RMP1-14 experienced complete tumor regression. In contrast, 100% of mice treated with an anti-mPD-1 RMP1-14-hIL-2 F42K, Y45R, V69R fusion protein experienced durable, long-term tumor regression. Mice treated with various combinations of the individual components of anti-mPD-1 RMP1-14-hIL-2 F42K, Y45R, V69R fusion protein, including either anti-mPD-1 RMP1-14 combined with hIL-2 free cytokine (administered at a dose and regimen equivalent to a therapeutic dose in humans) or anti-mPD-1 RMP1-14 combined with a non-targeted anti-KLH-hIL-2 F42K, Y45R, V69R fusion protein did not recapitulate the efficacy seen with anti-mPD-1 RMP1-14-hIL-2 F42K, Y45R, V69R. These data demonstrate that targeting a reduced potency hIL-2 to PD-1 expressing cells significantly improves anti-tumor efficacy relative to an anti-PD-1 receptor antagonist and that the activity of the fusion protein is not due to the additive effects of the molecule's individual components.

### Example 24: Evaluation of Protective Anti-Tumor Immunity Induced by Surrogate Anti-mPD-1 RMP1-14-hIL-2 F42K, Y45R, V69R in the MC38 Colo-Rectal Tumor Model

Mice that had undergone a complete tumor regression in the primary tumor study described in Example 23 and that had survived to day 50 were subjected to a secondary tumor challenge without any additional drug therapy. For tumor re-challenge, mice were implanted on the left flank contralateral to the location of the primary tumor with 5×10⁵ MC38 tumor cells. As a control group, 10 age-matched tumor naïve mice were also implanted with MC38 tumor cells.

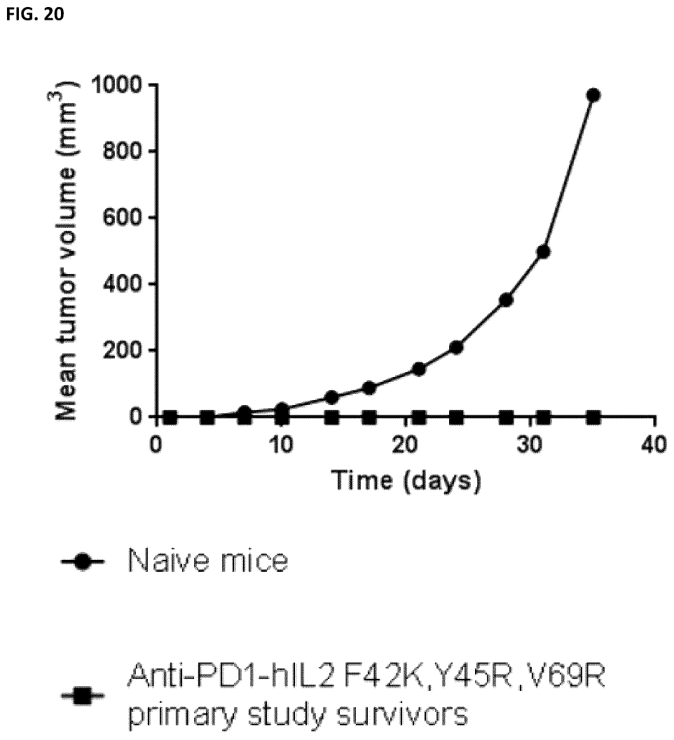

FIG. 20 shows that all mice that had previously undergone a complete tumor regression in a prior primary tumor study and had survived to day 50 after treatment with anti-mPD-1 RMP1-14-hIL-2 F42K, Y45R, V69R were completely protected from secondary tumor development. In contrast, all tumor-naïve mice implanted with MC38 tumor cells went on to develop tumors that rapidly reached study endpoint of tumor volume of 100 mm³. The development of protective anti-tumor immunity in the absence of continued drug therapy suggests that anti-mPD-1 RMP1-14-hIL-2 F42K, Y45R, V69R induced an anti-tumor memory T cell response.

### Example 25: Evaluation of the Compatibility of Combination Therapy with Surrogate Anti-mPD-1 RMP1-30-hIL-2 F42K, Y45R, V69R and Anti-mPD-1 RMP1-14 Antibodies in a Mouse MC38 Colorectal Tumor Model

To evaluate the compatibility of the disclosed immunoconjugates for use in combination therapy with antagonistic anti-PD-1 antibodies, surrogate antagonist anti-mouse PD-1 antibodies and surrogate immunoconjugates were used because antagonistic anti-human PD-1 antibodies do not bind mouse PD-1 and the modified IL-2 proteins do not work on mouse IL-2 receptors. Ten week old female C57BL/6NCrl mice were injected into the right flank with 5×10⁵ syngeneic MC38 colorectal carcinoma cells. When tumors reached 80-120 mm³, mice were sorted into cohorts (10 mice/group) and treatment began on day 1 of study. All agents were dosed intraperitoneally, once weekly for 4 weeks, starting on day 1. Anti-mPD-1 RMP1-30-hIL-2 F42K, Y45R, V69R was dosed at 1 mg/kg and anti-mPD-1 RMP1-14 at 2 mg/kg. Tumor size was measured with calipers twice weekly for the duration of the study. The study endpoint was a mean tumor weight of 1500 mm³ in the vehicle-treated control group, or 45 days, whichever came first. Endpoint sampling was conducted over two days due to the high number of mice needed to be sampled.

Both test agents were generated using a mouse IgG2b Fc region with a single N297A amino-acid substitution at position 297, which prevents glycosylation of the Fc region and significantly reduces any Fc region-mediated immune effector function, thereby preventing cellular depletion in vivo. Anti-mPD-1 RMP1-14 is a monoclonal antibody antagonist of the mouse PD-1 receptor (Matsumoto, J Immunol 172:2530-2541, 2004). Anti-mPD-1 RMP1-30-hIL-2 F42K/Y45R/V69R is a bi-functional fusion protein consisting of the monoclonal RMP1-30 antibody non-antagonist of the mouse PD-1 receptor fused at its C-terminus via a flexible six amino-acid glycine/serine linker to hIL-2 F42K/Y45R/V69R (SEQ ID NO: 621) that is a IL-2 variant with reduced potency on IL-2 receptors in mice. This molecule was designed to target the reduced potency hIL-2 variant directly to PD-1 expressing T cells in vivo in mice.

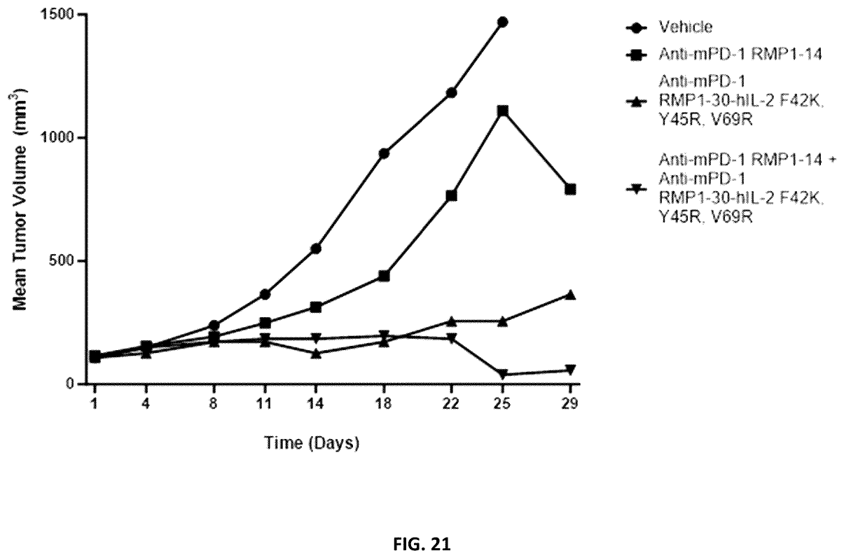

Results are presented in FIG. 21. Tumors growing in vehicle-treated mice reached study endpoint by day 25. In contrast, mice treated with anti-mPD-1 RMP1-14 or anti-mPD-1 RMP1-30-hIL-2 F42K/Y45R/V69R experienced partial tumor regression, with a better outcome in the anti-mPD-1 RMP1-30-hIL-2 F42K/Y45R/V69R group. The effect in the combination group led to the most effective tumor volume reduction. This data demonstrated that neither antibody interfered with the other's ability to bind to PD-1, and when administered in combination could achieve greater efficacy than each agent individually.

This data, combined with the data presented herein demonstrating that the disclosed immunoconjugates bind PD-1 in the presence of antagonistic anti-PD-1 antibodies and the data presented herein demonstrating that the disclosed immunoconjugates are designed to deliver the modified hIL-2 protein to IL-2 receptors rather than antagonizing PD-1, shows that the disclosed immunoconjugates and antagonistic anti-PD-1 antibodies are effective for use as a combination therapy to reduce tumor volume.

### Example 26: Combination Therapy with Anti-Human PD-1 (hPD-1) Antibody-Modified Human Interleukin-2 (hIL-2) Immunoconjugates and Antagonistic Anti-PD-1 Antibodies

The data in Example 25 above demonstrates that a greater therapeutic efficacy can be achieved by combining anti-hPD-1 antibody-modified hIL-2 immunoconjugates with antagonistic anti-PD-1 antibodies. A single-dose study for preliminary safety assessment will be performed in Cynomolgus monkeys with: 1) the disclosed immunoconjugates with and without nivolumab; and 2) the disclosed immunoconjugates with and without pembrolizumab. Sampling at time-points up to 360 hours following infusion will be performed. Adverse effects, gross toxicities, body weight loss, and lethality will be evaluated. Follow-up studies using higher doses of immunoconjugate and/or antagonistic anti-PD-1 antibody will be performed.

Those skilled in the art will appreciate that numerous changes and modifications can be made to the preferred embodiments disclosed herein and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

TABLE 28
Exemplary Antibodies

| Antibody Name | Heavy chain | Light chain |
|---|---|---|
| Anti-hPD-1 | Anti-hPD-1 #1-mIgG2b- | Anti-hPD-1 #1-mKappa LC |
| #1-mIgG2b-N297A | N297A HC | (SEQ ID NO: 349) |
| | (SEQ ID NO: 348) | |
| Anti-hPD-1 | Anti-hPD-1 #2-mIgG2b- | Anti-hPD-1 #2-mKappa LC |
| #2-mIgG2b-N297A | N297A HC | (SEQ ID NO: 351) |
| | (SEQ ID NO: 350) | |
| hIL-2 Nterm light | 1H3-hIgG1 HC | hIL-2-df-1H3-hkappa LC |
| chain df | (SEQ ID NO: 379) | (SEQ ID NO: 356) |
| hIL-2 Nterm light | 1H3-hIgG1 HC | hIL-2-L6-1H3-hkappa LC |
| chain L6 fusion | (SEQ ID NO: 379) | (SEQ ID NO: 357) |
| hIL-2 Nterm heavy | hIL-2-df-1H3-hIgG1 HC | 1H3-hKappa LC |
| chain df | (SEQ ID NO: 358) | (SEQ ID NO: 374) |
| hIL-2 Nterm heavy | hIL-2-L6-1H3-hIgG1 HC | 1H3-hKappa LC |
| chain L6 fusion | (SEQ ID NO: 359) | (SEQ ID NO: 374) |
| hIL-2 Cterm heavy | 1H3-hIgG1-df-hIL-2 HC | 1H3-hKappa LC |
| chain df | (SEQ ID NO: 360) | (SEQ ID NO: 374) |
| hIL-2 Cterm heavy | 1H3-hIgG1-L6-hIL-2 HC | 1H3-hKappa LC |
| chain L6 fusion | (SEQ ID NO: 361) | (SEQ ID NO: 374) |
| hIL-2 Cterm light | 1H3-hIgG1 HC | 1H3-hKappa-df-hIL-2 |
| chain df | (SEQ ID NO: 379) | (WT) LC |
| | | (SEQ ID NO: 362) |
| hIL-2 Cterm light | 1H3-hIgG1 HC | 1H3-hKappa-L6-hIL-2 |
| chain L6 fusion | (SEQ ID NO: 379) | (WT) LC |
| | | (SEQ ID NO: 363) |
| hCD25-L20-hIL-2 Nterm heavy | hCD25-L20-hIL-2-df- | 1H3-hKappa LC |
| chain df | 1H3-hIgG1 HC | (SEQ ID NO: 374) |
| | (SEQ ID NO: 365) | |
| hCD25-L20-hIL-2 Nterm heavy | hCD25-L20-hIL-2-L6- | 1H3-hKappa LC |
| chain L6 fusion | 1H3-hIgG1 HC | (SEQ ID NO: 374) |
| | (SEQ ID NO: 366) | |
| hCD25-L20-hIL-2 Nterm light | 1H3-hIgG1 HC | hCD25-L20-hIL-2-df- |
| chain df | (SEQ ID NO: 379) | 1H3-hKappa LC |
| | | (SEQ ID NO: 367) |
| hCD25-L20-hIL-2 Nterm light | 1H3-hIgG1 HC | hCD25-L20-hIL-2-L6- |
| chain L6 fusion | (SEQ ID NO: 379) | 1H3-hKappa LC |
| | | (SEQ ID NO: 368) |
| hCD25-L20-hIL-2 Cterm heavy | 1H3-hIgG1-df-hCD25- | 1H3-hKappa LC |
| chain df | L20-hIL-2 HC | (SEQ ID NO: 374) |
| | (SEQ ID NO: 369) | |
| hCD25-L20-hIL-2 Cterm heavy | 1H3-hIgG1-L6-hCD25- | 1H3-hKappa LC |
| chain L6 fusion | L20-hIL-2 HC | (SEQ ID NO: 374) |
| | (SEQ ID NO: 370) | |
| hCD25-L20-hIL-2 Cterm light | 1H3-hIgG1 HC | 1H3-hKappa-df-hCD25- |
| chain df | (SEQ ID NO: 379) | L20-hIL-2 LC |
| | | (SEQ ID NO: 371) |
| hCD25-L20-hIL-2 Cterm light | 1H3-hIgG1 HC | 1H3-hKappa-L6-hCD25- |
| chain L6 fusion | (SEQ ID NO: 379) | L20-hIL-2 LC |
| | | (SEQ ID NO: 372) |
| 2D12-mIgG1-D265A- | 2D12-mIgG1-D265A- | 2D12-mKappa LC |
| L6-hIL-2 | L6-hIL-2 HC | (SEQ ID NO: 376) |
| | (SEQ ID NO: 375) | |
| 2H7-hIgG4 | 2H7-hIgG4 HC | 2H7-hKappa LC |
| | (SEQ ID NO: 424) | (SEQ ID NO: 425) |
| C51E6-5-hIgG4 | C51E6-5-hIgG4 HC | C51E6-5-hKappa LC |
| | (SEQ ID NO: 426) | (SEQ ID NO: 427) |
| A2-hIgG4 | A2-hIgG4 HC | A2-hLambda LC |
| | (SEQ ID NO: 428) | (SEQ ID NO: 429) |
| H7-632-hIgG1-LAGA | H7-632 HC | H7-632 LC |
| | (SEQ ID NO: 414) | (SEQ ID NO: 415) |
| 2H7-hIgG4-df-hIL-2 | 2H7-hIgG4-df-hIL-2 | 2H7-hKappa LC |
| (D20A/R38E) | (D20A/R38E) HC | (SEQ ID NO: 425) |
| | (SEQ ID NO: 430) | |
| C51E6-5-hIgG4-L6-hIL-2 | C51E6-5-hIgG4-L6-hIL-2 | C51E6-5-hKappa LC |
| (D20A/R38E) | (D20A/R38E) HC | (SEQ ID NO: 427) |
| | (SEQ ID NO: 432) | |
| A2-hIgG4-df-hIL-2 | A2-hIgG4-df-hIL-2 | A2-hLambda LC |
| (D20A/R38E) | (D20A/R38E) HC | (SEQ ID NO: 429) |
| | (SEQ ID NO: 433) | |
| 1H3-hIgG4-df-hIL-2 | 1H3-hIgG4-df-hIL-2 | 1H3-hKappa LC |
| (D20A/R38E) | (D20A/R38E) HC | (SEQ ID NO: 374) |
| | (SEQ ID NO: 434) | |
| 2H7-hIgG4-df-hIL-2 | 2H7-hIgG4-df-hIL-2 | 2H7-hKappa LC |
| (T3A/D20A/R38E/C125A) | (T3A/D20A/R38E/C125A) HC | (SEQ ID NO: 425) |
| | (SEQ ID NO: 435) | |
| OMC.1.B6-hIgG4 | OMC.1.B6-hIgG4 HC | OMC.1.B6-hLambda LC |
| | (SEQ ID NO: 438) | (SEQ ID NO: 439) |
| OMC.2.C6-hIgG4 | OMC.2.C6-hIgG4 HC | OMC.2.C6-hLambda LC |
| | (SEQ ID NO: 440) | (SEQ ID NO: 441) |
| OMC.1.D6-hIgG4 | OMC.1.D6-hIgG4 HC | OMC.1.D6-hLambda LC |
| | (SEQ ID NO: 442) | (SEQ ID NO: 443) |
| D12-hIgG4 | D12-hIgG4 HC | D12-hLambda LC |
| | (SEQ ID NO: 444) | (SEQ ID NO: 445) |
| G12-hIgG4 | G12-hIgG4 HC | G12-hLambda LC |
| | (SEQ ID NO: 446) | (SEQ ID NO: 447) |
| Abz1mod-hIgG4 | Abz1mod-hIgG4 HC | Abz1mod-hKappa LC |
| | (SEQ ID NO: 449) | (SEQ ID NO: 450) |
| Anti-hPD-1 #1-hIgG4- | Anti-hPD-1 #1-hIgG4-L6- | Anti-hPD-1 #1-hKappa |
| L6-hIL-2 | hIL-2 (D20A/R38E) | (SEQ ID NO: 452) |
| (D20A/R38E) | (SEQ ID NO: 451) | |
| OMC.1.B6-hIgG4-L6-hIL-2 | OMC.1.B6-hIgG4-L6-hIL-2 | OMC.1.B6-hLambda LC |
| (D20A/R38E) | (D20A/R38E) HC | (SEQ ID NO: 439) |
| | (SEQ ID NO: 453) | |
| OMC.2.C6-hIgG4-L6-hIL-2 | OMC.2.C6-hIgG4-L6-hIL-2 | OMC.2.C6-hLambda LC |
| (D20A/R38E) | (D20A/R38E) HC | (SEQ ID NO: 441) |
| | (SEQ ID NO: 454) | |
| OMC.1.D6-hIgG4-L6-hIL-2 | OMC.1.D6-hIgG4-L6-hIL-2 | OMC.1.D6-hLambda LC |
| (D20A/R38E) | (D20A/R38E) HC | (SEQ ID NO: 443) |
| | (SEQ ID NO: 455) | |
| D12-hIgG4-df-hIL-2 | D12-hIgG4-df-hIL-2 | D12-hLambda LC |
| (D20A/R38E) | (D20A/R38E) HC | (SEQ ID NO: 445) |
| | (SEQ ID NO: 456) | |
| G12-hIgG4-df-hIL-2 | G12-hIgG4-df-hIL-2 | G12-hLambda LC |
| (D20A/R38E) | (D20A/R38E) HC | (SEQ ID NO: 447) |
| | (SEQ ID NO: 457) | |
| 2H7-hIgG4-LE | 2H7-hIgG4-LE HC | 2H7-hKappa LC |
| | (SEQ ID NO: 458) | (SEQ ID NO: 425) |
| 2H7-hIgG4-LAGA | 2H7-hIgG4-LAGA HC | 2H7-hKappa LC |
| | (SEQ ID NO: 459) | (SEQ ID NO: 425) |
| OMC476pH7-hIgG4 | OMC476pH7-hIgG4 HC | OMC476pB11.H7 LC |
| | (SEQ ID NO: 461) | (SEQ ID NO: 462) |
| OMC476pB11-hIgG4 | OMC476pB11-hIgG4 HC | OMC476pB11.H7 LC |
| | (SEQ ID NO: 463) | (SEQ ID NO: 462) |
| OMC476pG10-hIgG4 | OMC476pG10-hIgG4 HC | OMC476pG10.H10 LC |
| | (SEQ ID NO: 464) | (SEQ ID NO: 466) |
| OMC476pH10-hIgG4 | OMC476pH10-hIgG4 HC | OMC476pG10.H10 LC |
| | (SEQ ID NO: 465) | (SEQ ID NO: 466) |
| OMC476pE4-hIgG4 | OMC476pE4-hIgG4 HC | OMC476pE4 LC |
| | (SEQ ID NO: 467) | (SEQ ID NO: 468) |
| J110-hIgG1 | J110-hIgG1 HC | J110-hKappa LC |
| | (SEQ ID NO: 469) | (SEQ ID NO: 470) |
| 2H7-hIgG1-LAGA-df-hIL-2 | 2H7-hIgG1-LAGA-df-hIL-2 | 2H7-hKappa LC |
| (T3A/D20A/R38E/C125A) | (T3A/D20A/R38E/C125A) HC | (SEQ ID NO: 425) |
| | (SEQ ID NO: 471) | |
| 2H7-hIgG4-LE-df-hIL-2 | 2H7-hIgG4-LE-df-hIL-2 | 2H7-hKappa LC |
| (T3A/D20A/R38E/C125A) | (T3A/D20A/R38E/C125A) HC | (SEQ ID NO: 425) |
| | (SEQ ID NO: 472) | |
| 2H7-hIgG4-LAGA-df-hIL-2 | 2H7-hIgG4-LAGA-df-hIL-2 | 2H7-hKappa LC |
| (T3A/D20A/R38E/C125A) | (T3A/D20A/R38E/C125A) HC | (SEQ ID NO: 425) |
| | (SEQ ID NO: 473) | |
| 2H7-hIgG1-LAGA-df-hIL-2 | 2H7-hIgG1-LAGA-df-hIL-2 | 2H7-hKappa LC |
| (T3A/R38E/192K/C125A) | (T3A/R38E/192K/C125A) HC | (SEQ ID NO: 425) |
| | (SEQ ID NO: 474) | |
| hIgG4-LE-df-hIL-2 | hIgG4-LE-df-hIL-2 | 2H7-hKappa LC |
| (T3A/R38E/192K/C125A) | (T3A/R38E/192K/C125A) HC | (SEQ ID NO: 425) |
| | (SEQ ID NO: 475) | |
| 2H7-hIgG4-LAGA-df-hIL-2 | 2H7-hIgG4-LAGA-df-hIL-2 | 2H7-hKappa LC |
| (T3A/R38E/192K/C125A) | (T3A/R38E/192K/C125A) HC | (SEQ ID NO: 425) |
| | (SEQ ID NO: 476) | |
| 2H7-hIgG1-LAGA-df-hIL-2 | 2H7-hIgG1-LAGA-df-hIL-2 | 2H7-hKappa LC |
| (T3A/R38E/D84K/C125A) | (T3A/R38E/D84K/C125A) HC | (SEQ ID NO: 425) |
| | (SEQ ID NO: 477) | |
| 2H7-hIgG4-LE-df-hIL-2 | 2H7-hIgG4-LE-df-hIL-2 | 2H7-hKappa LC |
| (T3A/R38E/D84K/C125A) | (T3A/R38E/D84K/C125A) HC | (SEQ ID NO: 425) |
| | (SEQ ID NO: 478) | |
| 2H7-hIgG4-LAGA-df-hIL-2 | 2H7-hIgG4-LAGA-df-hIL-2 | 2H7-hKappa LC |
| (T3A/R38E/D84K/C125A) | (T3A/R38E/D84K/C125A) HC | (SEQ ID NO: 425) |
| | (SEQ ID NO: 479) | |
| 1H3-hIgG4-df-hIL-2 | 1H3-hIgG4-df-hIL-2 | 1H3-hKappa LC |
| (WT) | (WT) HC | (SEQ ID NO: 374) |
| | (SEQ ID NO: 480) | |
| 1H3-hIgG4-L6-hIL-2 | 1H3-hIgG4-L6-hIL-2 | 1H3-hKappa LC |
| (WT) | (WT) HC | (SEQ ID NO: 374) |
| | (SEQ ID NO: 481) | |
| 1H3-hIgG4-df-hIL-2 | 1H3-hIgG4 HC | 1H3-hKappa-df-hIL-2 |
| (WT) LC fusion | (SEQ ID NO: 482) | (WT) LC |
| | | (SEQ ID NO: 362) |
| 1H3-hIgG4-L6-hIL-2 | 1H3-hIgG4 HC | 1H3-hKappa-L6-hIL-2 |
| (WT) LC fusion | (SEQ ID NO: 482) | (WT) LC |
| | | (SEQ ID NO: 363) |
| 1H3-hIgG4-L6-hIL-2 | 1H3-hIgG4-L6-hIL-2 | 1H3-hKappa LC |
| (D20Y) | (D20Y) HC | (SEQ ID NO: 374) |
| | (SEQ ID NO: 485) | |
| 1H3-hIgG4-df-hIL-2 | 1H3-hIgG4-df-hIL-2 | 1H3-hKappa LC |
| (D20Y) | (D20Y) HC | (SEQ ID NO: 374) |
| | (SEQ ID NO: 486) | |
| 1H3-hIgG1-df-hIL-2 | 1H3-hIgG1-df-hIL-2 | 1H3-hKappa LC |
| (D20Y) | (D20Y) HC | (SEQ ID NO: 374) |
| | (SEQ ID NO: 487) | |
| 1H3-hIgG4-L6-hIL-2 | 1H3-hIgG4-L6-hIL-2 | 1H3-hKappa LC |
| (D20A/R38P) | (D20A/R38P) HC | (SEQ ID NO: 374) |
| | (SEQ ID NO: 488) | |
| 1H3-hIgG4-L6-hIL-2 | 1H3-hIgG4-L6-hIL-2 | 1H3-hKappa LC |
| (D20A/R38S) | (D20A/R38S) HC | (SEQ ID NO: 374) |
| | (SEQ ID NO: 489) | |
| 1H3-hIgG4-L6-hIL-2 | 1H3-hIgG4-L6-hIL-2 | 1H3-hKappa LC |
| (D20A/R38D) | (D20A/R38D) HC | (SEQ ID NO: 374) |
| | (SEQ ID NO: 490) | |
| 1H3-hIgG4-L6-hIL-2 | 1H3-hIgG4-L6-hIL-2 | 1H3-hKappa LC |
| (D20A/R38Q/E95A) | (D20A/R38Q/E95A) HC | (SEQ ID NO: 374) |
| | (SEQ ID NO: 491) | |
| 1H3-hIgG4-L6-hIL-2 | 1H3-hIgG4-L6-hIL-2 | 1H3-hKappa LC |
| (D20A/F42H/E95A) | (D20A/F42H/E95A) HC | (SEQ ID NO: 374) |
| | (SEQ ID NO: 492) | |
| 1H3-hIgG4-L6-hIL-2 | 1H3-hIgG4-L6-hIL-2 | 1H3-hKappa LC |
| (R38D/192D) | (R38D/192D) HC | (SEQ ID NO: 374) |
| | (SEQ ID NO: 493) | |
| 1H3-hIgG4-L6-hIL-2 | 1H3-hIgG4-L6-hIL-2 | 1H3-hKappa LC |
| (R38E/192D) | (R38E/192D) HC | (SEQ ID NO: 374) |
| | (SEQ ID NO: 494) | |
| 1H3-hIgG4-L6-hIL-2 | 1H3-hIgG4-L6-hIL-2 | 1H3-hKappa LC |
| (F42H/192D) | (F42H/192D) HC | (SEQ ID NO: 374) |
| | (SEQ ID NO: 495) | |
| 1H3-hIgG4-L6-hIL-2 | 1H3-hIgG4-L6-hIL-2 | 1H3-hKappa LC |
| (D20A/R38E) | (D20A/R38E) HC | (SEQ ID NO: 374) |
| | (SEQ ID NO: 496) | |
| 1H3-hIgG4-L6-hIL-2 | 1H3-hIgG4-L6-hIL-2 | 1H3-hKappa LC |
| (T3A/D20A/R38E) | (T3A/D20A/R38E) HC | (SEQ ID NO: 374) |
| | (SEQ ID NO: 497) | |
| 1H3-hIgG4-L6-hIL-2 | 1H3-hIgG4-L6-hIL-2 | 1H3-hKappa LC |
| (D20A/R38E/C125A) | (D20A/R38E/C125A) HC | (SEQ ID NO: 374) |
| | (SEQ ID NO: 498) | |
| 1H3-hIgG4-L6-hIL-2 | 1H3-hIgG4-L6-hIL-2 | 1H3-hKappa LC |
| (T3A/D20A/R38E/C125A) | (T3A/D20A/R38E/C125A) HC | (SEQ ID NO: 374) |
| | (SEQ ID NO: 499) | |
| 1H3-hIgG1-L6-hIL-2 | 1H3-hIgG1-L6-hIL-2 | 1H3-hKappa LC |
| (D20A/R38E) | (D20A/R38E) HC | (SEQ ID NO: 374) |
| | (SEQ ID NO: 500) | |
| 1H3-hIgG1-L6-hIL-2 | 1H3-hIgG1-L6-hIL-2 | 1H3-hKappa LC |
| (T3A/D20A/R38E) | (T3A/D20A/R38E) HC | (SEQ ID NO: 374) |
| | (SEQ ID NO: 501) | |
| 1H3-hIgG1-L6-hIL-2 | 1H3-hIgG1-L6-hIL-2 | 1H3-hKappa LC |
| (D20A/R38E/C125A) | (D20A/R38E/C125A) HC | (SEQ ID NO: 374) |
| | (SEQ ID NO: 502) | |
| 1H3-hIgG4-df-hIL-2 | 1H3-hIgG4 HC | 1H3-hKappa-df-hIL-2 |
| (D20A/R38E) LC fusion | (SEQ ID NO: 482) | (D20A/R38E) LC |
| | | (SEQ ID NO: 503) |
| 1H3-hIgG4-L6-hIL-2 | 1H3-hIgG4 HC | 1H3-hKappa-L6-hIL-2 |
| (D20A/R38E) LC fusion | (SEQ ID NO: 482) | (D20A/R38E) LC |
| | | (SEQ ID NO: 504) |
| OMC476pB11-hIgG4-df-hIL-2 | OMC476pB11-hIgG4-df-hIL-2 | OMC476pB11.H7 LC |
| (D20A/R38E) | (D20A/R38E) HC | (SEQ ID NO: 462) |
| | (SEQ ID NO: 505) | |
| OMC476pE4-hIgG4-df-hIL-2 | OMC476pE4-hIgG4-df-hIL-2 | OMC476pE4 LC |
| (D20A/R38E) | (D20A/R38E) HC | (SEQ ID NO: 468) |
| | (SEQ ID NO: 506) | |
| OMC476pG10-hIgG4-df-hIL-2 | OMC476pG10-hIgG4-df-hIL-2 | OMC476pG10.H10 LC |
| (D20A/R38E) | (D20A/R38E) HC | (SEQ ID NO: 466) |
| | (SEQ ID NO: 507) | |
| OMC476pH10-hIgG4-df-hIL-2 | OMC476pH10-hIgG4-df-hIL-2 | OMC476pG10.H10 LC |
| (D20A/R38E) | (D20A/R38E) HC | (SEQ ID NO: 466) |
| | (SEQ ID NO: 508) | |
| A2-hIgG4-df-hIL-2 | A2-hIgG4-df-hIL-2 | A2-hLambda LC |
| (D20A/F42A) | (D20A/F42A) HC | (SEQ ID NO: 429) |
| | (SEQ ID NO: 509) | |
| A2-hIgG4-df-hIL-2 | A2-hIgG4-df-hIL-2 | A2-hLambda LC |
| (D20A/F42S) | (D20A/F42S) HC | (SEQ ID NO: 429) |
| | (SEQ ID NO: 510) | |
| A2-hIgG4-df-hIL-2 | A2-hIgG4-df-hIL-2 | A2-hLambda LC |
| (D20S/R38E) | (D20S/R38E) HC | (SEQ ID NO: 429) |
| | (SEQ ID NO: 511) | |
| A2-hIgG4-df-hIL-2 | A2-hIgG4-df-hIL-2 | A2-hLambda LC |
| (F42A/N88R) | (F42A/N88R) HC | (SEQ ID NO: 429) |
| | (SEQ ID NO: 512) | |
| A2-hIgG4-df-hIL-2 | A2-hIgG4-df-hIL-2 | A2-hLambda LC |
| (F42I/192D) | (F421/192D) HC | (SEQ ID NO: 429) |
| | (SEQ ID NO: 513) | |
| A2-hIgG4-df-hIL-2 | A2-hIgG4-df-hIL-2 | A2-hLambda LC |
| (F42Q/192D) | (F42Q/192D) HC | (SEQ ID NO: 429) |
| | (SEQ ID NO: 514) | |
| A2-hIgG4-df-hIL-2 | A2-hIgG4-df-hIL-2 | A2-hLambda LC |
| (F42T/192D) | (F42T/192D) HC | (SEQ ID NO: 429) |
| | (SEQ ID NO: 515) | |
| A2-hIgG4-df-hIL-2 | A2-hIgG4-df-hIL-2 | A2-hLambda LC |
| (F42W/192D) | (F42W/192D) HC | (SEQ ID NO: 429) |
| | (SEQ ID NO: 516) | |
| A2-hIgG4-df-hIL-2 | A2-hIgG4-df-hIL-2 | A2-hLambda LC |
| (R38E/D84K) | (R38E/D84K) HC | (SEQ ID NO: 429) |
| | (SEQ ID NO: 517) | |
| A2-hIgG4-df-hIL-2 | A2-hIgG4-df-hIL-2 | A2-hLambda LC |
| (R38E/192K) | (R38E/192K) HC | (SEQ ID NO: 429) |
| | (SEQ ID NO: 518) | |
| C51E6-5-hIgG4-df-hIL-2 | C51E6-5-hIgG4-df-hIL-2 | C51E6-5-hKappa LC |
| (D20A/R38E) | (D20A/R38E) HC | (SEQ ID NO: 427) |
| | (SEQ ID NO: 519) | |
| C51E6-5-hIgG4-LE-df-hIL-2 | C51E6-5-hIgG4-LE-df-hIL-2 | C51E6-5-hKappa LC |
| (T3A/D20A/R38E/C125A) | (T3A/D20A/R38E/C125A) HC | (SEQ ID NO: 427) |
| | (SEQ ID NO: 520) | |
| C51E6-5-hIgG4-LAGA-df-hIL-2 | C51E6-5-hIgG4-LAGA-df-hIL-2 | C51E6-5-hKappa LC |
| (T3A/D20A/R38E/C125A) | (D20A/R38E) HC | (SEQ ID NO: 427) |
| | (SEQ ID NO: 521) | |
| 2A3.H7-hIgG4-df-hIL-2 | 2H7-hIgG4-df-hIL-2 | 2A3-hKappa LC |
| (D20A/R38E) | (D20A/R38E) HC | (SEQ ID NO: 523) |
| | (SEQ ID NO: 430) | |
| 1H9-hIgG4-df-hIL-2 | 1H9-hIgG4-df-hIL-2 | 1H9-hKappa LC |
| (D20A/R38E) | (D20A/R38E) HC | (SEQ ID NO: 525) |
| | (SEQ ID NO: 524) | |
| 1D5-hIgG4-df-hIL-2 | 1D5-hIgG4-df-hIL-2 | 1D5-hKappa LC |
| (D20A/R38E) | (D20A/R38E) HC | (SEQ ID NO: 527) |
| | (SEQ ID NO: 526) | |
| 1D5-hIgG4-LE-df-hIL-2 | 1D5-hIgG4-LE-df-hIL-2 | 1D5-hKappa LC |
| (T3A/D20A/R38E/C125A) | (T3A/D20A/R38E/C125A) HC | (SEQ ID NO: 527) |
| | (SEQ ID NO: 528) | |
| 1D5-hIgG4-LAGA-df-hIL-2 | 1D5-hIgG4-LAGA-df-hIL-2 | 1D5-hKappa LC |
| (T3A/D20A/R38E/C125A) | (T3A/D20A/R38E/C125A) HC | (SEQ ID NO: 527) |
| | (SEQ ID NO: 529) | |
| 2H7-hIgG1-df-hIL-2 | 2H7-hIgG1-df-hIL-2 | 2H7-hKappa LC |
| (T3A/D20A/R38E/C125A) | (T3A/D20A/R38E/C125A) HC | (SEQ ID NO: 425) |
| | (SEQ ID NO: 530) | |
| 2H7-hIgG1-LE-df-hIL-2 | 2H7-hIgG1-LE-df-hIL-2 | 2H7-hKappa LC |
| (T3A/D20A/R38E/C125A) | (T3A/D20A/R38E/C125A) HC | (SEQ ID NO: 425) |
| | (SEQ ID NO: 531) | |
| 2H7-hIgG1-LE-df-hIL-2 | 2H7-hIgG1-LE-df-hIL-2 | 2H7-hKappa LC |
| (T3A/R38E/D84K/C125A) | (T3A/R38E/D84K/C125A) HC | (SEQ ID NO: 425) |
| | (SEQ ID NO: 533) | |
| 2H7-hIgG4-df-hIL-2 | 2H7-hIgG4-df-hIL-2 | 2H7-hKappa LC |
| (T3A/R38E/D84K/C125A) | (T3A/R38E/D84K/C125A) HC | (SEQ ID NO: 425) |
| | (SEQ ID NO: 534) | |
| 2H7-hIgG1-df-hIL-2 | 2H7-hIgG1-df-hIL-2 | 2H7-hKappa LC |
| (T3A/R38E/192K/C125A) | (T3A/R38E/192K/C125A) HC | (SEQ ID NO: 425) |
| | (SEQ ID NO: 535) | |
| 2H7-hIgG1-LE-df-hIL-2 | 2H7-hIgG1-LE-df-hIL-2 | 2H7-hKappa LC |
| (T3A/R38E/192K/C125A) | (T3A/R38E/192K/C125A) HC | (SEQ ID NO: 425) |
| | (SEQ ID NO: 536) | |
| 2H7-hIgG4-df-hIL-2 | 2H7-hIgG4-df-hIL-2 | 2H7-hKappa LC |
| (T3A/R38E/192K/C125A) | (T3A/R38E/192K/C125A) HC | (SEQ ID NO: 425) |
| | (SEQ ID NO: 537) | |
| 2H7-hIgG1-LAGA-df-hIL2 | 2H7-hIgG1-LAGA-df-hIL2 | 2H7-hKappa LC |
| (T3A/D20S/R38E/C125A) | (T3A/D20S/R38E/C125A) HC | (SEQ ID NO: 425) |
| | (SEQ ID NO: 538) | |
| 2H7-hIgG1-LAGA-df-hIL2 | 2H7-hIgG1-LAGA-df-hIL2 | 2H7-hKappa LC |
| (T3A/R38E/D84F/C125A) | (T3A/R38E/D84F/C125A) HC | (SEQ ID NO: 425) |
| | (SEQ ID NO: 539) | |
| 2H7-hIgG1-LAGA-df-hIL2 | 2H7-hIgG1-LAGA-df-hIL2 | 2H7-hKappa LC |
| (T3A/R38E/192R/C125A) | (T3A/R38E/192R/C125A) HC | (SEQ ID NO: 425) |
| | (SEQ ID NO: 540) | |
| 2H7-hIgG1-LAGA-df-hIL2 | 2H7-hIgG1-LAGA-df-hIL2 | 2H7-hKappa LC |
| (T3A/R38E/192E/C125A) | (T3A/R38E/192E/C125A) HC | (SEQ ID NO: 425) |
| | (SEQ ID NO: 541) | |
| 2H7-hIgG1-LAGA-df-hIL2 | 2H7-hIgG1-LAGA-df-hIL2 | 2H7-hKappa LC |
| (T3A/R38E/192S/C125A) | (T3A/R38E/192S/C125A) HC | (SEQ ID NO: 425) |
| | (SEQ ID NO: 542) | |
| 2H7-hIgG1-LAGA-df-hIL2 | 2H7-hIgG1-LAGA-df-hIL2 | 2H7-hKappa LC |
| (T3A/R38E/192D/C125A) | (T3A/R38E/192D/C125A) HC | (SEQ ID NO: 425) |
| | (SEQ ID NO: 543) | |
| 2H7-hIgG1-LAGA-df-hIL2 | 2H7-hIgG1-LAGA-df-hIL2 | 2H7-hKappa LC |
| (T3A/H16E/R38E/C125A) | (T3A/H16E/R38E/C125A) HC | (SEQ ID NO: 425) |
| | (SEQ ID NO: 544) | |
| 1H3-hIgG1-L6-hIL-2 | 1H3-hIgG1-L6-hIL-2 | 1H3-hKappa LC |
| (T3A/D20A/R38E/C125A) | (T3A/D20A/R38E/C125A) HC | (SEQ ID NO: 374) |
| | (SEQ ID NO: 545) | |
| 1H3-hIgG1-LAGA-df-hIL-2 | 1H3-hIgG1-LAGA-df-hIL-2 | 1H3-hKappa LC |
| (T3A/D20A/R38E/C125A) | (T3A/D20A/R38E/C125A) HC | (SEQ ID NO: 374) |
| | (SEQ ID NO: 546) | |
| C51E6-5-hIgG4/k-LE | C51E6-5-hIgG4/k-LE HC | C51E6-5-hKappa LC |
| | (SEQ ID NO: 547) | (SEQ ID NO: 427) |
| C51E6-5-hIgG4/k-LAGA | C51E6-5-hIgG4/k-LAGA HC | C51E6-5-hKappa LC |
| | (SEQ ID NO: 548) | (SEQ ID NO: 427) |
| C51E6-5-hIgG4/k-LEPG | C51E6-5-hIgG4/k-LEPG HC | C51E6-5-hKappa LC |
| | (SEQ ID NO: 549) | (SEQ ID NO: 427) |
| C51E6-5-hIgG4/k-df-hIL-2 | C51E6-5-hIgG4/k-df-hIL-2 | C51E6-5-hKappa LC |
| (T3A/D20A/R38E/C125A) | (T3A/D20A/R38E/C125A) HC | (SEQ ID NO: 427) |
| | (SEQ ID NO: 550) | |
| C51E6-5-hIgG4/k-LEPG-hIL-2 | C51E6-5-hIgG4/k-LEPG-hIL-2 | C51E6-5-hKappa LC |
| (T3A/D20A/R38E/C125A) | (T3A/D20A/R38E/C125A) HC | (SEQ ID NO: 427) |
| | (SEQ ID NO: 551) | |
| A2-hIgG4/k-LE | A2-hIgG4/k-LE HC | A2-hLambda LC |
| | (SEQ ID NO: 552) | (SEQ ID NO: 429) |
| A2-hIgG4/k-LAGA | A2-hIgG4/k-LAGA HC | A2-hLambda LC |
| | (SEQ ID NO: 553) | (SEQ ID NO: 429) |
| A2-hIgG4/k-LEPG | A2-hIgG4/k-LEPG HC | A2-hLambda LC |
| | (SEQ ID NO: 554) | (SEQ ID NO: 429) |
| A2-hIgG4/k-df-hIL-2 | A2-hIgG4/k-df-hIL-2 | A2-hLambda LC |
| (T3A/D20A/R38E/C125A) | (T3A/D20A/R38E/C125A) HC | (SEQ ID NO: 429) |
| | (SEQ ID NO: 555) | |
| A2-hIgG4/k-LE-df-hIL-2 | A2-hIgG4/k-LE-df-hIL-2 | A2-hLambda LC |
| (T3A/D20A/R38E/C125A) | (T3A/D20A/R38E/C125A) HC | (SEQ ID NO: 429) |
| | (SEQ ID NO: 556) | |
| A2-hIgG4/k-LAGA-df-hIL-2 | A2-hIgG4/k-LAGA-df-hIL-2 | A2-hLambda LC |
| (T3A/D20A/R38E/C125A) | (T3A/D20A/R38E/C125A) HC | (SEQ ID NO: 429) |
| | (SEQ ID NO: 557) | |
| A2-hIgG4/k-LEPG-df-hIL-2 | A2-hIgG4/k-LEPG-df-hIL-2 | A2-hLambda LC |
| (T3A/D20A/R38E/C125A) | (T3A/D20A/R38E/C125A) HC | (SEQ ID NO: 429) |
| | (SEQ ID NO: 558) | |
| Anti-CD20-hIgG1/k | Anti-CD20-hIgG1/k HC | Anti-CD20-hKappa LC |
| | (SEQ ID NO: 560) | (SEQ ID NO: 562) |
| Anti-CD20-hIgG1/k-LAGA | Anti-CD20-hIgG1/k-LAGA HC | Anti-CD20-hKappa LC |
| | (SEQ ID NO: 561) | (SEQ ID NO: 562) |
| 1H3-hIgG1-LAGA-df-hIL-2 | 1H3-hIgG1-LAGA-df-hIL-2 | 1H3-hKappa LC |
| (T3A/C125A) | (T3A/C125A) HC | (SEQ ID NO: 374) |
| | (SEQ ID NO: 563) | |
| anti-mPD-1 RMP1-14 | anti-mPD-1 RMP1-14 | anti-mPD-1 RMP1-14 |
| mIgG2b-N297A | mIgG2b-N297A HC | mKappa LC |
| | (SEQ ID NO: 564) | (SEQ ID NO: 566) |
| anti-mPD-1 RMP1-14 | anti-mPD-1 RMP1-14 | anti-mPD-1 RMP1-14 |
| mIgG2b-N297A-L6-hIL-2 | mIgG2b-N297A-L6-hIL-2 | mKappa LC |
| (F42K/Y45R/V69R) | (F42K/Y45R/V69R) HC | (SEQ ID NO: 566) |
| | (SEQ ID NO: 565) | |
| anti-mPD-1 RMP1-30 | anti-mPD-1 RMP1-30 | anti-mPD-1 RMP1-30 |
| mIgG2b-N297A | mIgG2b-N297A HC | mKappa LC |
| | (SEQ ID NO: 567) | (SEQ ID NO: 568) |
| anti-mPD-1 RMP1-30 | anti-mPD-1 RMP1-30 | anti-mPD-1 RMP1-30 |
| mIgG2b-N297A-L6-hIL-2 | mIgG2b-N297A-L6-hIL-2 | mKappa LC |
| (F42K/Y45R/V69R) | (F42K/Y45R/V69R) HC | (SEQ ID NO: 568) |
| | (SEQ ID NO: 569) | |
| anti-KLH-C3-mIgG2b- | anti-KLH-C3-mIgG2b- | KLH-C3-mKappa LC |
| N297A-L6-hIL-2 | N297A-L6-hIL-2 | (SEQ ID NO: 571) |
| (F42K/Y45R/V69R) | (F42K/Y45R/V69R) HC | |
| | (SEQ ID NO: 570) | |
| 2D12-hIgG1-L6-hIL-2 | 2D12-hIgG1-L6-hIL-2 HC | 2D12-hKappa LC |
| | (SEQ ID NO: 572) | (SEQ ID NO: 573) |
| 1H9-hIgG4 | 1H9-hIgG4 HC | 1H9-hKappa LC |
| | (SEQ ID NO: 576) | (SEQ ID NO: 525) |
| 1D5-hIgG4 | 1D5-hIgG4 HC | 1D5-hKappa LC |
| | (SEQ ID NO: 577) | (SEQ ID NO: 527) |
| 2A3.H7-hIgG4 | 2H7-hIgG4 HC | 2A3-hKappa LC |
| | (SEQ ID NO: 424) | (SEQ ID NO: 523) |
| H7-02-hIgG1-LAGA-df-hIL-2 | H7-02-hIgG1-LAGA-df-hIL-2 | H7-02-hKappa LC |
| (T3A/D20A/R38E/C125A) | (T3A/D20A/R38E/C125A) HC | (SEQ ID NO: 583) |
| | (SEQ ID NO: 582) | |
| KLH-C3-hIgG4 | KLH-C3-hIgG4 HC | KLH-C3-hKappa LC |
| | (SEQ ID NO: 585) | (SEQ ID NO: 586) |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (E15A) | (E15A) HC | |
| | (SEQ ID NO: 587) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (D20I) | (D20I) HC | |
| | (SEQ ID NO: 588) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (D20S) | (D20S) HC | |
| | (SEQ ID NO: 589) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (D20H) | (D20H) HC | |
| | (SEQ ID NO: 590) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (D20W) | (D20W) HC | |
| | (SEQ ID NO: 591) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (D20Y) | (D20Y) HC | |
| | (SEQ ID NO: 592) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (D20R) | (D20R) HC | |
| | (SEQ ID NO: 593) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (D20F) | (D20F) HC | |
| | (SEQ ID NO: 594) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (D84K) | (D84K) HC | |
| | (SEQ ID NO: 595) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (S87A) | (S87A) HC | |
| | (SEQ ID NO: 596) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (N88Y) | (N88Y) HC | |
| | (SEQ ID NO: 597) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (N88D) | (N88D) HC | |
| | (SEQ ID NO: 598) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (N88R) | (N88R) HC | |
| | (SEQ ID NO: 599) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (N88E) | (N88E) HC | |
| | (SEQ ID NO: 600) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (N88F) | (N88F) HC | |
| | (SEQ ID NO: 601) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (N88I) | (N88I) HC | |
| | (SEQ ID NO: 602) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (192A) | (192A) HC | |
| | (SEQ ID NO: 603) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (E95A) | (E95A) HC | |
| | (SEQ ID NO: 604) | |
| 1H3-hIgG1-L6-hCD25 | 1H3-hIgG1-L6-hCD25 | 1H3-hkappa LC |
| (1-164)-L20-hIL-2 | (1-164)-L20-hIL-2 | (SEQ ID NO: 374) |
| (E95K) | (E95K) HC | |
| | (SEQ ID NO: 605) | |
| H7-02-hIgG4 | H7-02-hIgG4 HC | H7-02 hKappa LC |
| | (SEQ ID NO: 373) | (SEQ ID NO: 607) |
| H7-632-hIgG1-LAGA- | H7-632-hIgG1-LAGA-df-hIL-2 | H7-632 LC |
| df-hIL-2 | (T3A/C125A) HC | (SEQ ID NO: 415) |
| (T3A/C125A) | (SEQ ID NO: 431) | |
| 1H3-hIgG1-LAGA-L6-hIL-2 | 1H3-hIgG1-LAGA-L6-hIL-2 | 1H3-hKappa LC |
| (T3A/D20A/R38E/C125A) | (T3A/D20A/R38E/C125A) | (SEQ ID NO: 374) |
| | (SEQ ID NO: 522) | |
| 1H3-hIgG1 | 1H3-hIgG1 HC | 1H3-hKappa LC |
| | (SEQ ID NO: 379) | (SEQ ID NO: 374) |
| H7-767 | H7-767 HC | H7-632 LC |
| | (SEQ ID NO: 532) | (SEQ ID NO: 415) |
| Anti-hPD-1 #1 | Anti-hPD-1 #1 HC | Anti-hPD-1#1-hKappa |
| | (SEQ ID NO: 559) | (SEQ ID NO: 452) |
| Anti-hPD-1 #2 | Anti-hPD-1 #2 HC | Anti-hPD-1 #2 LC |
| | (SEQ ID NO: 578) | (SEQ ID NO: 579) |
| 2H7-hIgG4-LE-df-hIL-2 | 2H7-hIgG4-LE-df-hIL-2 | 2H7-hKappa LC |
| (T3A/R38E/192K/C125A) | (T3A/R38E/192K/C125A) HC | (SEQ ID NO: 425) |
| | (SEQ ID NO: 475) | |

TABLE 29
Sequences

| SEQ | | |
|---|---|---|
| ID | | |
| NO: | Name | Sequence |
| 1 | hIL-2 F42K | APTSSSTKKTQLQLEHLLLDLQMILNGINNYKNPKLTRMLTKKFYMPKKATELKHLQCLEEELKPLEEV |
| | | LNLAQSKNFHLRPRDLISNINVIVLELKGSETTEMCEYADETATIVEFLNRWITFCQSIISTLT |
| 2 | hIL-2 V69A | APTSSSTKKTQLQLEHLLLDLQMILNGINNYKNPKLTRMLTFKFYMPKKATELKHLQCLEEELKPLEEA |
| | | LNLAQSKNFHLRPRDLISNINVIVLELKGSETTEMCEYADETATIVEFLNRWITFCQSIISTLT |
| 3 | hIL-2 V69E | APTSSSTKKTQLQLEHLLLDLQMILNGINNYKNPKLTRMLTFKFYMPKKATELKHLQCLEEELKPLEEE |
| | | LNLAQSKNFHLRPRDLISNINVIVLELKGSETTFMCEYADETATIVEFLNRWITFCQSIISTLT |
| 4 | hIL-2 V69F | APTSSSTKKTQLQLEHLLLDLQMILNGINNYKNPKLTRMLTFKFYMPKKATELKHLQCLEEELKPLEEF |
| | | LNLAQSKNFHLRPRDLISNINVIVLELKGSETTFMCEYADETATIVEFLNRWITFCQSIISTLT |
| 5 | hIL-2 V69G | APTSSSTKKTQLQLEHLLLDLQMILNGINNYKNPKLTRMLTFKFYMPKKATELKHLQCLEEELKPLEEG |
| | | LNLAQSKNFHLRPRDLISNINVIVLELKGSETTEMCEYADETATIVEFLNRWITFCQSIISTLT |
| 6 | hIL-2 V69H | APTSSSTKKTQLQLEHLLLDLQMILNGINNYKNPKLTRMLTFKFYMPKKATELKHLQCLEEELKPLEEH |
| | | LNLAQSKNFHLRPRDLISNINVIVLELKGSETTEMCEYADETATIVEFLNRWITFCQSIISTLT |
| 7 | hIL-2 V69I | APTSSSTKKTQLQLEHLLLDLQMILNGINNYKNPKLTRMLTFKFYMPKKATELKHLQCLEEELKPLEEI |
| | | LNLAQSKNFHLRPRDLISNINVIVLELKGSETTFMCEYADETATIVEFLNRWITFCQSIISTLT |
| 8 | hIL-2 V69K | APTSSSTKKTQLQLEHLLLDLQMILNGINNYKNPKLTRMLTFKFYMPKKATELKHLQCLEEELKPLEEK |
| | | LNLAQSKNFHLRPRDLISNINVIVLELKGSETTFMCEYADETATIVEFLNRWITFCQSIISTLT |
| 9 | hIL-2 V69L | APTSSSTKKTQLQLEHLLLDLQMILNGINNYKNPKLTRMLTFKFYMPKKATELKHLQCLEEELKPLEEL |
| | | LNLAQSKNFHLRPRDLISNINVIVLELKGSETTEMCEYADETATIVEFLNRWITFCQSIISTLT |
| 10 | hIL-2 V69M | APTSSSTKKTQLQLEHLLLDLQMILNGINNYKNPKLTRMLTFKFYMPKKATELKHLQCLEEELKPLEEM |
| | | LNLAQSKNFHLRPRDLISNINVIVLELKGSETTEMCEYADETATIVEFLNRWITFCQSIISTLT |
| 11 | hIL-2 V69Q | APTSSSTKKTQLQLEHLLLDLQMILNGINNYKNPKLTRMLTFKFYMPKKATELKHLQCLEEELKPLEEQ |
| | | LNLAQSKNFHLRPRDLISNINVIVLELKGSETTEMCEYADETATIVEFLNRWITFCQSIISTLT |
| 12 | hIL-2 V69S | APTSSSTKKTQLQLEHLLLDLQMILNGINNYKNPKLTRMLTFKFYMPKKATELKHLQCLEEELKPLEES |
| | | LNLAQSKNFHLRPRDLISNINVIVLELKGSETTEMCEYADETATIVEFLNRWITFCQSIISTLT |
| 13 | hIL-2 V69T | APTSSSTKKTQLQLEHLLLDLQMILNGINNYKNPKLTRMLTFKFYMPKKATELKHLQCLEEELKPLEET |
| | | LNLAQSKNFHLRPRDLISNINVIVLELKGSETTEMCEYADETATIVEFLNRWITFCQSIISTLT |
| 14 | hIL-2 V69W | APTSSSTKKTQLQLEHLLLDLQMILNGINNYKNPKLTRMLTFKFYMPKKATELKHLQCLEEELKPLEEW |
| | | LNLAQSKNFHLRPRDLISNINVIVLELKGSETTFMCEYADETATIVEFLNRWITFCQSIISTLT |
| 15 | hIL-2 V69Y | APTSSSTKKTQLQLEHLLLDLQMILNGINNYKNPKLTRMLTFKFYMPKKATELKHLQCLEEELKPLEEY |
| | | LNLAQSKNFHLRPRDLISNINVIVLELKGSETTFMCEYADETATIVEFLNRWITFCQSIISTLT |
| 16 | hIL-2 | APTSSSTKKTQLQLEHLLLDLQMILNGINNYKNPKLTRMLTKKKYMPKKATELKHLQCLEEELKPLEEV |
| | F42K/F44K | LNLAQSKNFHLRPRDLISNINVIVLELKGSETTFMCEYADETATIVEFLNRWITFCQSIISTLT |
| 17 | hIL-2 | APTSSSTKKTQLQLEHLLLDLQMILNGINNYKNPKLTRMLTFKKRMPKKATELKHLQCLEEELKPLEEV |
| | F44K/Y45R | LNLAQSKNFHLRPRDLISNINVIVLELKGSETTFMCEYADETATIVEFLNRWITFCQSIISTLT |
| 18 | hIL-2 | APTSSSTKKTQLQLEHLLLDLQMILNGINNYKNPKLTRMLTKKFYMPKKATELKHLQCLEEELKPLEER |
| | F42K/V69R | LNLAQSKNFHLRPRDLISNINVIVLELKGSETTEMCEYADETATIVEFLNRWITFCQSIISTLT |
| 19 | hIL-2 | APTSSSTKKTQLQLEHLLLDLQMILNGINNYKNPKLTRMLTFKERMPKKATELKHLQCLEEELKPLEER |
| | Y45R/V69R | LNLAQSKNFHLRPRDLISNINVIVLELKGSETTFMCEYADETATIVEFLNRWITFCQSIISTLT |
| 20 | hIL-2 | APTSSSTKKTQLQLEHLLLDLQMILNGINNYKNPKLTRMLTKKKRMPKKATELKHLQCLEEELKPLEEV |
| | F42K/F44K/Y45R | LNLAQSKNFHLRPRDLISNINVIVLELKGSETTEMCEYADETATIVEFLNRWITFCQSIISTLT |
| 21 | hIL-2 | APTSSSTKKTQLQLEHLLLDLQMILNGINNYKNPKLTAMLTKKERMPKKATELKHLQCLEEELKPLEEV |
| | R38A/F42K/Y45R | LNLAQSKNFHLRPRDLISNINVIVLELKGSETTFMCEYADETATIVEFLNRWITFCQSIISTLT |
| 22 | hIL-2 | APTSSSTKKTQLQLEHLLLDLQMILNGINNYKNPKLTEMLTKKERMPKKATELKHLQCLEEELKPLEEV |
| | R38E/F42K/Y45R | LNLAQSKNFHLRPRDLISNINVIVLELKGSETTFMCEYADETATIVEFLNRWITFCQSIISTLT |
| 23 | hIL-2 | APTSSSTKKTQLQLEHLLLDLQMILNGINNYKNPKLTRMLTKEFRMPKKATELKHLQCLEEELKPLEEV |
| | K43E/F42K/Y45R | LNLAQSKNFHLRPRDLISNINVIVLELKGSETTFMCEYADETATIVEFLNRWITFCQSIISTLT |
| 24 | hIL-2 | APTSSSTKKTQLQLEHLLLDLQMILNGINNYKNPKLTRMLTKTFRMPKKATELKHLQCLEEELKPLEEV |
| | K43T/F42K/Y45R | LNLAQSKNFHLRPRDLISNINVIVLELKGSETTFMCEYADETATIVEFLNRWITFCQSIISTLT |
| 25 | hIL-2 | APTSSSTKKTQLQLEHLLLDLQMILNGINNYKNPKLTRMLTKKFRMPKKATELKHLQCLEEALKPLEEV |
| | F42K/Y45R/E62A | LNLAQSKNFHLRPRDLISNINVIVLELKGSETTEMCEYADETATIVEFLNRWITFCQSIISTLT |
| 26 | hIL-2 | APTSSSTKKTQLQLEHLLLDLQMILNGINNYKNPKLTRMLTKKERMPKKATELKHLQCLEEELKRLEEV |
| | P65R/F42K/Y45R | LNLAQSKNFHLRPRDLISNINVIVLELKGSETTFMCEYADETATIVEFLNRWITFCQSIISTLT |
| 27 | hIL-2 | APTSSSTKKTQLQLEHLLLDLQMILNGINNYKNPKLTRMLTKKFRMPKKATELKHLQCLEEELKSLEEV |
| | P65S/F42K/Y45R | LNLAQSKNFHLRPRDLISNINVIVLELKGSETTFMCEYADETATIVEFLNRWITFCQSIISTLT |
| 28 | hIL-2 | APTSSSTKKTQLQLEHLLLDLQMILNGINNYKNPKLTRMLTKKERMPKKATELKHLQCLEEELKPLEEA |
| | V69A/F42K/Y45R | LNLAQSKNFHLRPRDLISNINVIVLELKGSETTEMCEYADETATIVEFLNRWITFCQSIISTLT |
| 29 | hIL-2 | APTSSSTKKTQLQLEHLLLDLQMILNGINNYKNPKLTRMLTKKERMPKKATELKHLQCLEEELKPLEED |
| | V69D/F42K/Y45R | LNLAQSKNFHLRPRDLISNINVIVLELKGSETTEMCEYADETATIVEFLNRWITFCQSIISTLT |
| 30 | hIL-2 | APTSSSTKKTQLQLEHLLLDLQMILNGINNYKNPKLTRMLTKKFRMPKKATELKHLQCLEEELKPLEER |
| | V69R/F42K/Y45R | LNLAQSKNFHLRPRDLISNINVIVLELKGSETTFMCEYADETATIVEFLNRWITFCQSIISTLT |
| 31 | hIL-2 D20A | APTSSSTKKTQLQLEHLLLALQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 32 | hIL-2 D20N | APTSSSTKKTQLQLEHLLLNLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 33 | hIL-2 D20K | APTSSSTKKTQLQLEHLLLKLQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 34 | hIL-2 N88A | APTSSSTKKTQLQLEHLLLDLQMILNGINNYKNPKLTRMLTFKFYMPKKATELKHLQCLEEELKPLEEV |
| | | LNLAQSKNFHLRPRDLISAINVIVLELKGSETTEMCEYADETATIVEFLNRWITFCQSIISTLT |
| 35 | hIL-2 N88G | APTSSSTKKTQLQLEHLLLDLQMILNGINNYKNPKLTRMLTFKFYMPKKATELKHLQCLEEELKPLEEV |
| | | LNLAQSKNFHLRPRDLISGINVIVLELKGSETTEMCEYADETATIVEFLNRWITFCQSIISTLT |
| 36 | hIL-2 N88H | APTSSSTKKTQLQLEHLLLDLQMILNGINNYKNPKLTRMLTFKFYMPKKATELKHLQCLEEELKPLEEV |
| | | LNLAQSKNFHLRPRDLISHINVIVLELKGSETTFMCEYADETATIVEFLNRWITFCQSIISTLT |
| 37 | hIL-2 N88K | APTSSSTKKTQLQLEHLLLDLQMILNGINNYKNPKLTRMLTFKFYMPKKATELKHLQCLEEELKPLEEV |
| | | LNLAQSKNFHLRPRDLISKINVIVLELKGSETTEMCEYADETATIVEFLNRWITFCQSIISTLT |
| 38 | hIL-2 | APTSSSTKKTQLQLEHLLLALQMILNGINNYKNPKLTRMLTFKFYMPKKATELKHLQCLEEELKPLEEV |
| | D20A/D84A | LNLAQSKNFHLRPRALISNINVIVLELKGSETTEMCEYADETATIVEFLNRWITFCQSIISTLT |
| 39 | hIL-2 | APTSSSTKKTQLQLAHLLLALQMILNGINNYKNPKLTRMLTFKFYMPKKATELKHLQCLEEELKPLEEV |
| | D20A/E15A | LNLAQSKNFHLRPRDLISNINVIVLELKGSETTFMCEYADETATIVEFLNRWITFCQSIISTLT |
| 40 | hIL-2 | APTSSSTKKTQLQLEHLLLALQMILNGINNYKNPKLTRMLTFKFYMPKKATELKHLQCLEEELKPLEEV |
| | D20A/E95A | LNLAQSKNFHLRPRDLISNINVIVLALKGSETTFMCEYADETATIVEFLNRWITFCQSIISTLT |
| 41 | hIL-2 | APTSSSTKKTQLQLEHLLLALQMILNGINNYKNPKLTRMLTFKFYMPKKATELKHLQCLEEELKPLEEV |
| | D20A/N88A | LNLAQSKNFHLRPRDLISAINVIVLELKGSETTFMCEYADETATIVEFLNRWITFCQSIISTLT |
| 42 | hIL-2 | APTSSSTKKTQLQLEHLLLALQMILNGINNYKNPKLTRMLTFKFYMPKKATELKHLQCLEEELKPLEEV |
| | D20A/S87A | LNLAQSKNFHLRPRDLIANINVIVLELKGSETTFMCEYADETATIVEFLNRWITFCQSIISTLT |
| 43 | hIL-2 | APTSSSTKKTQLQLEHLLLDLQMILNGINNYKNPKLTRMLTFKFYMPKKATELKHLQCLEEELKPLEEV |
| | D84A/N88A | LNLAQSKNFHLRPRALISAINVIVLELKGSETTFMCEYADETATIVE FLNRWITFCQSIISTLT |
| 44 | hIL-2 | APTSSSTKKTQLQLAHLLLDLQMILNGINNYKNPKLTRMLTFKFYMPKKATELKHLQCLEEELKPLEEV |
| | E15A/N88A | LNLAQSKNFHLRPRDLISAINVIVLELKGSETTFMCEYADETATIVEFLNRWITFCQSIISTLT |
| 45 | hIL-2 | APTSSSTKKTQLQLEHLLLDLQMILNGINNYKNPKLTRMLTFKFYMPKKATELKHLQCLEEELKPLEEV |
| | S87A/N88A | LNLAQSKNFHLRPRDLIAAINVIVLELKGSETTFMCEYADETATIVEFLNRWITFCQSIISTLT |
| 46 | hIL-2 R38A | APTSSSTKKTQLQLEHLLLDLQMILNGINNYKNPKLTAMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 47 | hIL-2 R38D | APTSSSTKKTQLQLEHLLLDLQMILNGINNYKNPKLTDMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 48 | hIL-2 R38E | APTSSSTKKTQLQLEHLLLDLQMILNGINNYKNPKLTEMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 49 | hIL-2 R38Q | APTSSSTKKTQLQLEHLLLDLQMILNGINNYKNPKLTQMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 50 | hIL-2 F42R | APTSSSTKKTQLQLEHLLLDLQMILNGINNYKNPKLTRMLTRKFYMPKKATELKHLQCLEEELKPLEEV |
| | | LNLAQSKNFHLRPRDLISNINVIVLELKGSETTEMCEYADETATIVEFLNRWITFCQSIISTLT |
| 51 | hIL-2 F42A | APTSSSTKKTQLQLEHLLLDLQMILNGINNYKNPKLTRMLTAKFYMPKKATELKHLQCLEEELKPLEEV |
| | | LNLAQSKNFHLRPRDLISNINVIVLELKGSETTFMCEYADETATIVEFLNRWITFCQSIISTLT |
| 52 | hIL-2 F42D | APTSSSTKKTQLQLEHLLLDLQMILNGINNYKNPKLTRMLTDKFYMPKKATELKHLQCLEEELKPLEEV |
| | | LNLAQSKNFHLRPRDLISNINVIVLELKGSETTFMCEYADETATIVEFLNRWITFCQSIISTLT |
| 53 | hIL-2 F42H | APTSSSTKKTQLQLEHLLLDLQMILNGINNYKNPKLTRMLTHKFYMPKKATELKHLQCLEEELKPLEEV |
| | | LNLAQSKNFHLRPRDLISNINVIVLELKGSETTEMCEYADETATIVEFLNRWITFCQSIISTLT |
| 54 | hIL-2 K43A | APTSSSTKKTQLQLEHLLLDLQMILNGINNYKNPKLTRMLTFAFYMPKKATELKHLQCLEEELKPLEEV |
| | | LNLAQSKNFHLRPRDLISNINVIVLELKGSETTFMCEYADETATIVEFLNRWITFCQSIISTLT |
| 55 | hIL-2 K43E | APTSSSTKKTQLQLEHLLLDLQMILNGINNYKNPKLTRMLTFEFYMPKKATELKHLQCLEEELKPLEEV |
| | | LNLAQSKNFHLRPRDLISNINVIVLELKGSETTFMCEYADETATIVEFLNRWITFCQSIISTLT |
| 56 | hIL-2 K43Q | APTSSSTKKTQLQLEHLLLDLQMILNGINNYKNPKLTRMLTFQFYMPKKATELKHLQCLEEELKPLEEV |
| | | LNLAQSKNFHLRPRDLISNINVIVLELKGSETTEMCEYADETATIVEFLNRWITFCQSIISTLT |
| 57 | hIL-2 Y45A | APTSSSTKKTQLQLEHLLLDLQMILNGINNYKNPKLTRMLTFKFAMPKKATELKHLQCLEEELKPLEEV |
| | | LNLAQSKNFHLRPRDLISNINVIVLELKGSETTEMCEYADETATIVEFLNRWITFCQSIISTLT |
| 58 | hIL-2 Y45K | APTSSSTKKTQLQLEHLLLDLQMILNGINNYKNPKLTRMLTFKFKMPKKATELKHLQCLEEELKPLEEV |
| | | LNLAQSKNFHLRPRDLISNINVIVLELKGSETTFMCEYADETATIVEFLNRWITFCQSIISTLT |
| 59 | hIL-2 Y45S | APTSSSTKKTQLQLEHLLLDLQMILNGINNYKNPKLTRMLTFKFSMPKKATELKHLQCLEEELKPLEEV |
| | | LNLAQSKNFHLRPRDLISNINVIVLELKGSETTEMCEYADETATIVEFLNRWITFCQSIISTLT |
| 60 | hIL-2 Y45R | APTSSSTKKTQLQLEHLLLDLQMILNGINNYKNPKLTRMLTFKERMPKKATELKHLQCLEEELKPLEEV |
| | | LNLAQSKNFHLRPRDLISNINVIVLELKGSETTFMCEYADETATIVEFLNRWITFCQSIISTLT |
| 61 | hIL-2 E61A | APTSSSTKKTQLQLEHLLLDLQMILNGINNYKNPKLTRMLTFKFYMPKKATELKHLQCLEAELKPLEEV |
| | | LNLAQSKNFHLRPRDLISNINVIVLELKGSETTFMCEYADETATIVEFLNRWITFCQSIISTLT |
| 62 | hIL-2 E61R | APTSSSTKKTQLQLEHLLLDLQMILNGINNYKNPKLTRMLTFKFYMPKKATELKHLQCLERELKPLEEV |
| | | LNLAQSKNFHLRPRDLISNINVIVLELKGSETTFMCEYADETATIVEFLNRWITFCQSIISTLT |
| 63 | hIL-2 E61K | APTSSSTKKTQLQLEHLLLDLQMILNGINNYKNPKLTRMLTFKFYMPKKATELKHLQCLEKELKPLEEV |
| | | LNLAQSKNFHLRPRDLISNINVIVLELKGSETTFMCEYADETATIVEFLNRWITFCQSIISTLT |
| 64 | hIL-2 E62A | APTSSSTKKTQLQLEHLLLDLQMILNGINNYKNPKLTRMLTFKFYMPKKATELKHLQCLEEALKPLEEV |
| | | LNLAQSKNFHLRPRDLISNINVIVLELKGSETTFMCEYADETATIVEFLNRWITFCQSIISTLT |
| 65 | hIL-2 E62R | APTSSSTKKTQLQLEHLLLDLQMILNGINNYKNPKLTRMLTFKFYMPKKATELKHLQCLEERLKPLEEV |
| | | LNLAQSKNFHLRPRDLISNINVIVLELKGSETTEMCEYADETATIVEFLNRWITFCQSIISTLT |
| 66 | hIL-2 E62K | APTSSSTKKTQLQLEHLLLDLQMILNGINNYKNPKLTRMLTFKFYMPKKATELKHLQCLEEKLKPLEEV |
| | | LNLAQSKNFHLRPRDLISNINVIVLELKGSETTEMCEYADETATIVEFLNRWITFCQSIISTLT |
| 67 | hIL-2 E62Y | APTSSSTKKTQLQLEHLLLDLQMILNGINNYKNPKLTRMLTFKFYMPKKATELKHLQCLEEYLKPLEEV |
| | | LNLAQSKNFHLRPRDLISNINVIVLELKGSETTFMCEYADETATIVEFLNRWITFCQSIISTLT |
| 68 | hIL-2 E68Y | APTSSSTKKTQLQLEHLLLDLQMILNGINNYKNPKLTRMLTFKFYMPKKATELKHLQCLEEELKPLEYV |
| | | LNLAQSKNFHLRPRDLISNINVIVLELKGSETTFMCEYADETATIVEFLNRWITFCQSIISTLT |
| 69 | hIL-2 E68A | APTSSSTKKTQLQLEHLLLDLQMILNGINNYKNPKLTRMLTFKFYMPKKATELKHLQCLEEELKPLEAV |
| | | LNLAQSKNFHLRPRDLISNINVIVLELKGSETTFMCEYADETATIVEFLNRWITFCQSIISTLT |
| 70 | hIL-2 E68K | APTSSSTKKTQLQLEHLLLDLQMILNGINNYKNPKLTRMLTFKFYMPKKATELKHLQCLEEELKPLEKV |
| | | LNLAQSKNFHLRPRDLISNINVIVLELKGSETTEMCEYADETATIVEFLNRWITFCQSIISTLT |
| 71 | hIL-2 E68R | APTSSSTKKTQLQLEHLLLDLQMILNGINNYKNPKLTRMLTFKFYMPKKATELKHLQCLEEELKPLERV |
| | | LNLAQSKNFHLRPRDLISNINVIVLELKGSETTEMCEYADETATIVEFLNRWITFCQSIISTLT |
| 72 | hIL-2 E68L | APTSSSTKKTQLQLEHLLLDLQMILNGINNYKNPKLTRMLTFKFYMPKKATELKHLQCLEEELKPLELV |
| | | LNLAQSKNFHLRPRDLISNINVIVLELKGSETTFMCEYADETATIVEFLNRWITFCQSIISTLT |
| 73 | hIL-2 L72Y | APTSSSTKKTQLQLEHLLLDLQMILNGINNYKNPKLTRMLTFKFYMPKKATELKHLQCLEEELKPLEEV |
| | | LNYAQSKNFHLRPRDLISNINVIVLELKGSETTEMCEYADETATIVEFLNRWITFCQSIISTLT |
| 74 | hIL-2 L72R | APTSSSTKKTQLQLEHLLLDLQMILNGINNYKNPKLTRMLTFKFYMPKKATELKHLQCLEEELKPLEEV |
| | | LNRAQSKNFHLRPRDLISNINVIVLELKGSETTFMCEYADETATIVEFLNRWITFCQSIISTLT |
| 75 | hIL-2 L72A | APTSSSTKKTQLQLEHLLLDLQMILNGINNYKNPKLTRMLTFKFYMPKKATELKHLQCLEEELKPLEEV |
| | | LNAAQSKNFHLRPRDLISNINVIVLELKGSETTFMCEYADETATIVEFLNRWITFCQSIISTLT |
| 76 | hIL-2 L72D | APTSSSTKKTQLQLEHLLLDLQMILNGINNYKNPKLTRMLTFKFYMPKKATELKHLQCLEEELKPLEEV |
| | | LNDAQSKNFHLRPRDLISNINVIVLELKGSETTFMCEYADETATIVEFLNRWITFCQSIISTLT |
| 77 | hIL-2 L72H | APTSSSTKKTQLQLEHLLLDLQMILNGINNYKNPKLTRMLTFKFYMPKKATELKHLQCLEEELKPLEEV |
| | | LNHAQSKNFHLRPRDLISNINVIVLELKGSETTEMCEYADETATIVEFLNRWITFCQSIISTLT |
| 78 | hIL-2 L72F | APTSSSTKKTQLQLEHLLLDLQMILNGINNYKNPKLTRMLTFKFYMPKKATELKHLQCLEEELKPLEEV |
| | | LNFAQSKNFHLRPRDLISNINVIVLELKGSETTEMCEYADETATIVEFLNRWITFCQSIISTLT |
| 79 | hIL-2 | APTSSSTKKTQLQLEHLLLDLQMILNGINNYKNPKLTDMLTFKFYMPKKATELKHLQCLERELKPLEEV |
| | R38D/E61R | LNLAQSKNFHLRPRDLISNINVIVLELKGSETTFMCEYADETATIVEFLNRWITFCQSIISTLT |
| 80 | hIL-2 | APTSSSTKKTQLQLEHLLLDLQMILNGINNYKNPKLTDMLTFEFYMPKKATELKHLQCLERELKPLEEV |
| | R38D/E61R/K43E | LNLAQSKNFHLRPRDLISNINVIVLELKGSETTFMCEYADETATIVEFLNRWITFCQSIISTLT |
| 81 | hIL-2 | APASSSTKKTQLQLEHLLLDLQMILNGINNYKNPKLTRMLTAKFAMPKKATELKHLQCLEEELKPLEEV |
| | T3A/F42A/Y45A/ | LNGAQSKNFHLRPRDLISNINVIVLELKGSETTEMCEYADETATIVEFLNRWITFAQSIISTLT |
| | L72G/C125A | |
| 82 | hIL-2 E15A | APTSSSTKKTQLQLAHLLLDLQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 83 | hIL-2 E15R | APTSSSTKKTQLQLRHLLLDLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 84 | hIL-2 E15K | APTSSSTKKTQLQLKHLLLDLQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 85 | hIL-2 H16A | APTSSSTKKTQLQLEALLLDLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 86 | hIL-2 H16Y | APTSSSTKKTQLQLEYLLLDLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 87 | hIL-2 H16E | APTSSSTKKTQLQLEELLLDLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 88 | hIL-2 L19A | APTSSSTKKTQLQLEHLLADLQMILNGINNYKNPKLTRMLTFKFYMPKKATELKHLOCLEEELKPLEEV |
| | | LNLAQSKNFHLRPRDLISNINVIVLELKGSETTEMCEYADETATIVEFLNRWITFCQSIISTLT |
| 89 | hIL-2 D20I | APTSSSTKKTQLQLEHLLLILQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 90 | hIL-2 D20S | APTSSSTKKTQLQLEHLLLSLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 91 | hIL-2 D20H | APTSSSTKKTQLQLEHLLLHLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 92 | hIL-2 D20T | APTSSSTKKTQLQLEHLLLTLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 93 | hIL-2 D20W | APTSSSTKKTQLQLEHLLLWLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 94 | hIL-2 D20Y | APTSSSTKKTQLQLEHLLLYLQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 95 | hIL-2 D20R | APTSSSTKKTQLQLEHLLLRLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCQSIISTLT |
| 96 | hIL-2 D20F | APTSSSTKKTQLQLEHLLLFLQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 97 | hIL-2 R81A | APTSSSTKKTQLQLEHLLLDLQMILNGINNYKNPKLTRMLTFKFYMPKKATELKHLQCLEEELKPLEEV |
| | | LNLAQSKNFHLAPRDLISNINVIVLELKGSETTFMCEYADETATIVEFLNRWITFCQSIISTLT |
| 98 | hIL-2 D84A | APTSSSTKKTQLQLEHLLLDLQMILNGINNYKNPKLTRMLTFKFYMPKKATELKHLQCLEEELKPLEEV |
| | | LNLAQSKNFHLRPRALISNINVIVLELKGSETTFMCEYADETATIVEFLNRWITFCQSIISTLT |
| 99 | hIL-2 D84R | APTSSSTKKTQLQLEHLLLDLQMILNGINNYKNPKLTRMLTFKFYMPKKATELKHLQCLEEELKPLEEV |
| | | LNLAQSKNFHLRPRRLISNINVIVLELKGSETTFMCEYADETATIVEFLNRWITFCQSIISTLT |
| 100 | hIL-2 D84K | APTSSSTKKTQLQLEHLLLDLQMILNGINNYKNPKLTRMLTFKFYMPKKATELKHLQCLEEELKPLEEV |
| | | LNLAQSKNFHLRPRKLISNINVIVLELKGSETTFMCEYADETATIVEFLNRWITFCQSIISTLT |
| 101 | hIL-2 S87A | APTSSSTKKTQLQLEHLLLDLQMILNGINNYKNPKLTRMLTFKFYMPKKATELKHLQCLEEELKPLEEV |
| | | LNLAQSKNFHLRPRDLIANINVIVLELKGSETTEMCEYADETATIVEFLNRWITFCQSIISTLT |
| 102 | hIL-2 N88Y | APTSSSTKKTQLQLEHLLLDLQMILNGINNYKNPKLTRMLTFKFYMPKKATELKHLQCLEEELKPLEEV |
| | | LNLAQSKNFHLRPRDLISYINVIVLELKGSETTFMCEYADETATIVEFLNRWITFCQSIISTLT |
| 103 | hIL-2 N88D | APTSSSTKKTQLQLEHLLLDLQMILNGINNYKNPKLTRMLTFKFYMPKKATELKHLQCLEEELKPLEEV |
| | | LNLAQSKNFHLRPRDLISDINVIVLELKGSETTFMCEYADETATIVEFLNRWITFCQSIISTLT |
| 104 | hIL-2 N88R | APTSSSTKKTQLQLEHLLLDLQMILNGINNYKNPKLTRMLTFKFYMPKKATELKHLQCLEEELKPLEEV |
| | | LNLAQSKNFHLRPRDLISRINVIVLELKGSETTEMCEYADETATIVEFLNRWITFCQSIISTLT |
| 105 | hIL-2 N88E | APTSSSTKKTQLQLEHLLLDLQMILNGINNYKNPKLTRMLTFKFYMPKKATELKHLQCLEEELKPLEEV |
| | | LNLAQSKNFHLRPRDLISEINVIVLELKGSETTFMCEYADETATIVEFLNRWITFCQSIISTLT |
| 106 | hIL-2 N88F | APTSSSTKKTQLQLEHLLLDLQMILNGINNYKNPKLTRMLTFKFYMPKKATELKHLQCLEEELKPLEEV |
| | | LNLAQSKNFHLRPRDLISFINVIVLELKGSETTFMCEYADETATIVEFLNRWITFCQSIISTLT |
| 107 | hIL-2 N88I | APTSSSTKKTQLQLEHLLLDLQMILNGINNYKNPKLTRMLTFKFYMPKKATELKHLQCLEEELKPLEEV |
| | | LNLAQSKNFHLRPRDLISIINVIVLELKGSETTEMCEYADETATIVEFLNRWITFCQSIISTLT |
| 108 | hIL-2 I92A | APTSSSTKKTQLQLEHLLLDLQMILNGINNYKNPKLTRMLTFKFYMPKKATELKHLQCLEEELKPLEEV |
| | | LNLAQSKNFHLRPRDLISNINVAVLELKGSETTFMCEYADETATIVEFLNRWITFCQSIISTLT |
| 109 | hIL-2 I92Y | APTSSSTKKTQLQLEHLLLDLQMILNGINNYKNPKLTRMLTFKFYMPKKATELKHLQCLEEELKPLEEV |
| | | LNLAQSKNFHLRPRDLISNINVYVLELKGSETTFMCEYADETATIVEFLNRWITFCQSIISTLT |
| 110 | hIL-2 I92S | APTSSSTKKTQLQLEHLLLDLQMILNGINNYKNPKLTRMLTFKFYMPKKATELKHLQCLEEELKPLEEV |
| | | LNLAQSKNFHLRPRDLISNINVSVLELKGSETTFMCEYADETATIVEFLNRWITFCQSIISTLT |
| 111 | hIL-2 I92F | APTSSSTKKTQLQLEHLLLDLQMILNGINNYKNPKLTRMLTFKFYMPKKATELKHLQCLEEELKPLEEV |
| | | LNLAQSKNFHLRPRDLISNINVFVLELKGSETTEMCEYADETATIVEFLNRWITFCQSIISTLT |
| 112 | hIL-2 I92R | APTSSSTKKTQLQLEHLLLDLQMILNGINNYKNPKLTRMLTFKFYMPKKATELKHLQCLEEELKPLEEV |
| | | LNLAQSKNFHLRPRDLISNINVRVLELKGSETTFMCEYADETATIVEFLNRWITFCQSIISTLT |
| 113 | hIL-2 I92D | APTSSSTKKTQLQLEHLLLDLQMILNGINNYKNPKLTRMLTFKFYMPKKATELKHLQCLEEELKPLEEV |
| | | LNLAQSKNFHLRPRDLISNINVDVLELKGSETTEMCEYADETATIVEFLNRWITFCQSIISTLT |
| 114 | hIL-2 I92E | APTSSSTKKTQLQLEHLLLDLQMILNGINNYKNPKLTRMLTFKFYMPKKATELKHLQCLEEELKPLEEV |
| | | LNLAQSKNFHLRPRDLISNINVEVLELKGSETTFMCEYADETATIVEFLNRWITFCQSIISTLT |
| 115 | hIL-2 E95A | APTSSSTKKTQLQLEHLLLDLQMILNGINNYKNPKLTRMLTFKFYMPKKATELKHLQCLEEELKPLEEV |
| | | LNLAQSKNFHLRPRDLISNINVIVLALKGSETTFMCEYADETATIVEFLNRWITFCQSIISTLT |
| 116 | hIL-2 E95R | APTSSSTKKTQLQLEHLLLDLQMILNGINNYKNPKLTRMLTFKFYMPKKATELKHLQCLEEELKPLEEV |
| | | LNLAQSKNFHLRPRDLISNINVIVLRLKGSETTFMCEYADETATIVEFLNRWITFCQSIISTLT |
| 117 | hIL-2 E95K | APTSSSTKKTQLQLEHLLLDLQMILNGINNYKNPKLTRMLTFKFYMPKKATELKHLQCLEEELKPLEEV |
| | | LNLAQSKNFHLRPRDLISNINVIVLKLKGSETTFMCEYADETATIVEFLNRWITFCQSIISTLT |
| 118 | hIL-2 | APTSSSTKKTQLQLEELLLYLQMILNGINNYKNPKLTRMLTFKFYMPKKATELKHLQCLEEELKPLEEV |
| | D20Y/H16E | LNLAQSKNFHLRPRDLISNINVIVLELKGSETTEMCEYADETATIVEFLNRWITFCQSIISTLT |
| 119 | hIL-2 | APTSSSTKKTQLQLEALLLYLQMILNGINNYKNPKLTRMLTFKFYMPKKATELKHLQCLEEELKPLEEV |
| | D20Y/H16A | LNLAQSKNFHLRPRDLISNINVIVLELKGSETTFMCEYADETATIVEFLNRWITFCQSIISTLT |
| 120 | hIL-2 | APTSSSTKKTQLQLEYLLLYLQMILNGINNYKNPKLTRMLTFKFYMPKKATELKHLQCLEEELKPLEEV |
| | D20Y/H16Y | LNLAQSKNFHLRPRDLISNINVIVLELKGSETTFMCEYADETATIVEFLNRWITFCQSIISTLT |
| 121 | hIL-2 | APTSSSTKKTQLQLEHLLLYLQMILNGINNYKNPKLTRMLTFKFYMPKKATELKHLQCLEEELKPLEEV |
| | D20Y/I92A | LNLAQSKNFHLRPRDLISNINVAVLELKGSETTFMCEYADETATIVEFLNRWITFCQSIISTLT |
| 122 | hIL-2 | APTSSSTKKTQLQLEHLLLYLQMILNGINNYKNPKLTRMLTFKFYMPKKATELKHLQCLEEELKPLEEV |
| | D20Y/I92S | LNLAQSKNFHLRPRDLISNINVSVLELKGSETTFMCEYADETATIVEFLNRWITFCQSIISTLT |
| 123 | hIL-2 | APTSSSTKKTQLQLEHLLLYLQMILNGINNYKNPKLTRMLTFKFYMPKKATELKHLQCLEEELKPLEEV |
| | D20Y/I92R | LNLAQSKNFHLRPRDLISNINVRVLELKGSETTFMCEYADETATIVEFLNRWITFCQSIISTLT |
| 124 | hIL-2 | APTSSSTKKTQLQLEHLLLYLQMILNGINNYKNPKLTRMLTFKFYMPKKATELKHLQCLEEELKPLEEV |
| | D20Y/E95R | LNLAQSKNFHLRPRDLISNINVIVLRLKGSETTFMCEYADETATIVEFLNRWITFCQSIISTLT |
| 125 | hIL-2 | APTSSSTKKTQLQLEHLLLYLQMILNGINNYKNPKLTRMLTFKFYMPKKATELKHLQCLEEELKPLEEV |
| | D20Y/E95A | LNLAQSKNFHLRPRDLISNINVIVLALKGSETTEMCEYADETATIVEFLNRWITFCQSIISTLT |
| 126 | hCD25 (1-164) | ELCDDDPPEIPHATFKAMAYKEGTMLNCECKRGFRRIKSGSLYMLCTGNSSHSSWDNQCQCTSSATRNT |
| | | TKQVTPQPEEQKERKTTEMQSPMQPVDQASLPGHCREPPPWENEATERIYHFVVGQMVYYQCVQGYRAL |
| | | HRGPAESVCKMTHGKTRWTQPQLICT |
| 127 | hIL-2 | APTSSSTKKTQLQLEHLLLALQMILNGINNYKNPKLTRMLTDKFYMPKKATELKHLQCLEEELKPLEEV |
| | F42D/D20A | LNLAQSKNFHLRPRDLISNINVIVLELKGSETTFMCEYADETATIVEFLNRWITFCQSIISTLT |
| 128 | hIL-2 | APTSSSTKKTQLQLEHLLLALQMILNGINNYKNPKLTRMLTRKFYMPKKATELKHLQCLEEELKPLEEV |
| | F42R/D20A | LNLAQSKNFHLRPRDLISNINVIVLELKGSETTEMCEYADETATIVEFLNRWITFCQSIISTLT |
| 129 | hIL-2 | APTSSSTKKTQLQLEHLLLALQMILNGINNYKNPKLTRMLTKKFYMPKKATELKHLQCLEEELKPLEEV |
| | F42K/D20A | LNLAQSKNFHLRPRDLISNINVIVLELKGSETTFMCEYADETATIVEFLNRWITFCQSIISTLT |
| 130 | hIL-2 | APTSSSTKKTQLQLEHLLLALQMILNGINNYKNPKLTRMLTAKFYMPKKATELKHLQCLEEELKPLEEV |
| | F42A/D20A | LNLAQSKNFHLRPRDLISNINVIVLELKGSETTEMCEYADETATIVEFLNRWITFCQSIISTLT |
| 131 | hIL-2 | APTSSSTKKTQLQLEHLLLALQMILNGINNYKNPKLTRMLTHKFYMPKKATELKHLQCLEEELKPLEEV |
| | F42H/D20A | LNLAQSKNFHLRPRDLISNINVIVLELKGSETTEMCEYADETATIVEFLNRWITFCQSIISTLT |
| 132 | hIL-2 | APTSSSTKKTQLQLEHLLLALQMILNGINNYKNPKLTRMLTFKFRMPKKATELKHLQCLEEELKPLEEV |
| | Y45R/D20A | LNLAQSKNFHLRPRDLISNINVIVLELKGSETTFMCEYADETATIVEFLNRWITFCQSIISTLT |
| 133 | hIL-2 | APTSSSTKKTQLQLEHLLLALQMILNGINNYKNPKLTRMLTFKFKMPKKATELKHLQCLEEELKPLEEV |
| | Y45K/D20A | LNLAQSKNFHLRPRDLISNINVIVLELKGSETTFMCEYADETATIVEFLNRWITFCQSIISTLT |
| 134 | hIL-2 | APTSSSTKKTQLQLEHLLLALQMILNGINNYKNPKLTNMLTFKFYMPKKATELKHLQCLEEELKPLEEV |
| | R38N/D20A | LNLAQSKNFHLRPRDLISNINVIVLELKGSETTFMCEYADETATIVEFLNRWITFCQSIISTLT |
| 135 | hIL-2 | APTSSSTKKTQLQLEHLLLALQMILNGINNYKNPKLTGMLTFKFYMPKKATELKHLQCLEEELKPLEEV |
| | R38G/D20A | LNLAQSKNFHLRPRDLISNINVIVLELKGSETTEMCEYADETATIVEFLNRWITFCQSIISTLT |
| 136 | hIL-2 | APTSSSTKKTQLQLEHLLLALQMILNGINNYKNPKLTHMLTFKFYMPKKATELKHLQCLEEELKPLEEV |
| | R38H/D20A | LNLAQSKNFHLRPRDLISNINVIVLELKGSETTFMCEYADETATIVEFLNRWITFCQSIISTLT |
| 137 | hIL-2 | APTSSSTKKTQLQLEHLLLALQMILNGINNYKNPKLTIMLTFKFYMPKKATELKHLQCLEEELKPLEEV |
| | R38I/D20A | LNLAQSKNFHLRPRDLISNINVIVLELKGSETTEMCEYADETATIVEFLNRWITFCQSIISTLT |
| 138 | hIL-2 | APTSSSTKKTQLQLEHLLLALQMILNGINNYKNPKLTLMLTFKFYMPKKATELKHLQCLEEELKPLEEV |
| | R38L/D20A | LNLAQSKNFHLRPRDLISNINVIVLELKGSETTFMCEYADETATIVEFLNRWITFCQSIISTLT |
| 139 | hIL-2 | APTSSSTKKTQLQLEHLLLALQMILNGINNYKNPKLTMMLTFKFYMPKKATELKHLQCLEEELKPLEEV |
| | R38M/D20A | LNLAQSKNFHLRPRDLISNINVIVLELKGSETTEMCEYADETATIVEFLNRWITFCQSIISTLT |
| 140 | hIL-2 | APTSSSTKKTQLQLEHLLLALQMILNGINNYKNPKLTFMLTFKFYMPKKATELKHLQCLEEELKPLEEV |
| | R38F/D20A | LNLAQSKNFHLRPRDLISNINVIVLELKGSETTEMCEYADETATIVEFLNRWITFCQSIISTLT |
| 141 | hIL-2 | APTSSSTKKTQLQLEHLLLALQMILNGINNYKNPKLTPMLTFKFYMPKKATELKHLQCLEEELKPLEEV |
| | R38P/D20A | LNLAQSKNFHLRPRDLISNINVIVLELKGSETTFMCEYADETATIVEFLNRWITFCQSIISTLT |
| 142 | hIL-2 | APTSSSTKKTQLQLEHLLLALQMILNGINNYKNPKLTSMLTFKFYMPKKATELKHLQCLEEELKPLEEV |
| | R38S/D20A | LNLAQSKNFHLRPRDLISNINVIVLELKGSETTEMCEYADETATIVEFLNRWITFCQSIISTLT |
| 143 | hIL-2 | APTSSSTKKTQLQLEHLLLALQMILNGINNYKNPKLTTMLTFKFYMPKKATELKHLQCLEEELKPLEEV |
| | R38T/D20A | LNLAQSKNFHLRPRDLISNINVIVLELKGSETTFMCEYADETATIVEFLNRWITFCQSIISTLT |
| 144 | hIL-2 | APTSSSTKKTQLQLEHLLLALQMILNGINNYKNPKLTWMLTFKFYMPKKATELKHLQCLEEELKPLEEV |
| | R38W/D20A | LNLAQSKNFHLRPRDLISNINVIVLELKGSETTEMCEYADETATIVEFLNRWITFCQSIISTLT |
| 145 | hIL-2 | APTSSSTKKTQLQLEHLLLALQMILNGINNYKNPKLTYMLTFKFYMPKKATELKHLQCLEEELKPLEEV |
| | R38Y/D20A | LNLAQSKNFHLRPRDLISNINVIVLELKGSETTFMCEYADETATIVEFLNRWITFCQSIISTLT |
| 146 | hIL-2 | APTSSSTKKTQLQLEHLLLALQMILNGINNYKNPKLTVMLTFKFYMPKKATELKHLQCLEEELKPLEEV |
| | R38V/D20A | LNLAQSKNFHLRPRDLISNINVIVLELKGSETTEMCEYADETATIVEFLNRWITFCQSIISTLT |
| 147 | hIL-2 | APTSSSTKKTQLQLEHLLLALQMILNGINNYKNPKLTAMLTFKFYMPKKATELKHLQCLEEELKPLEEV |
| | R38A/D20A | LNLAQSKNFHLRPRDLISNINVIVLELKGSETTEMCEYADETATIVEFLNRWITFCQSIISTLT |
| 148 | hIL-2 | APTSSSTKKTQLQLEHLLLALQMILNGINNYKNPKLTQMLTFKFYMPKKATELKHLQCLEEELKPLEEV |
| | R38Q/D20A | LNLAQSKNFHLRPRDLISNINVIVLELKGSETTFMCEYADETATIVEFLNRWITFCQSIISTLT |
| 149 | hIL-2 | APTSSSTKKTQLQLEHLLLALQMILNGINNYKNPKLTEMLTFKFYMPKKATELKHLQCLEEELKPLEEV |
| | D20A/R38E | LNLAQSKNFHLRPRDLISNINVIVLELKGSETTFMCEYADETATIVEFLNRWITFCQSIISTLT |
| 150 | hIL-2 | APTSSSTKKTQLQLEHLLLALQMILNGINNYKNPKLTDMLTFKFYMPKKATELKHLQCLEEELKPLEEV |
| | R38D/D20A | LNLAQSKNFHLRPRDLISNINVIVLELKGSETTEMCEYADETATIVEFLNRWITFCQSIISTLT |
| 151 | hIL-2 | APTSSSTKKTQLQLEHLLLALQMILNGINNYKNPKLTRMLTFEFYMPKKATELKHLQCLEEELKPLEEV |
| | K43E/D20A | LNLAQSKNFHLRPRDLISNINVIVLELKGSETTEMCEYADETATIVEFLNRWITFCQSIISTLT |
| 152 | hIL-2 | APTSSSTKKTQLQLEHLLLALQMILNGINNYKNPKLTRMLTFKFYMPKKATELKHLQCLEAELKPLEEV |
| | E61A/D20A | LNLAQSKNFHLRPRDLISNINVIVLELKGSETTFMCEYADETATIVEFLNRWITFCQSIISTLT |
| 153 | hIL-2 | APTSSSTKKTQLQLEHLLLALQMILNGINNYKNPKLTRMLTFKFYMPKKATELKHLQCLEEALKPLEEV |
| | E62A/D20A | LNLAQSKNFHLRPRDLISNINVIVLELKGSETTFMCEYADETATIVEFLNRWITFCQSIISTLT |
| 154 | hIL-2 | APTSSSTKKTQLQLEHLLLALQMILNGINNYKNPKLTRMLTFKFYMPKKATELKHLQCLEEYLKPLEEV |
| | E62Y/D20A | LNLAQSKNFHLRPRDLISNINVIVLELKGSETTEMCEYADETATIVEFLNRWITFCQSIISTLT |
| 155 | hIL-2 | APTSSSTKKTQLQLEHLLLALQMILNGINNYKNPKLTRMLTFKFYMPKKATELKHLQCLEEELKPLEEV |
| | L72D/D20A | LNDAQSKNFHLRPRDLISNINVIVLELKGSETTFMCEYADETATIVEFLNRWITFCQSIISTLT |
| 156 | hIL-2 | APTSSSTKKTQLQLEHLLLALQMILNGINNYKNPKLTRMLTFKFYMPKKATELKHLQCLEEELKPLEEV |
| | L72H/D20A | LNHAQSKNFHLRPRDLISNINVIVLELKGSETTFMCEYADETATIVEFLNRWITFCQSIISTLT |
| 157 | hIL-2 | APTSSSTKKTQLQLEHLLLALQMILNGINNYKNPKLTRMLTFKFYMPKKATELKHLQCLEEELKPLEEV |
| | L72R/D20A | LNRAQSKNFHLRPRDLISNINVIVLELKGSETTEMCEYADETATIVEFLNRWITFCQSIISTLT |
| 158 | hIL-2 | APTSSSTKKTQLQLEHLLLDLQMILNGINNYKNPKLTRMLTDKFYMPKKATELKHLQCLEEELKPLEEV |
| | F42D/I92D | LNLAQSKNFHLRPRDLISNINVDVLELKGSETTFMCEYADETATIVEFLNRWITFCQSIISTLT |
| 159 | hIL-2 | APTSSSTKKTQLQLEHLLLDLQMILNGINNYKNPKLTRMLTRKFYMPKKATELKHLQCLEEELKPLEEV |
| | F42R/I92D | LNLAQSKNFHLRPRDLISNINVDVLELKGSETTEMCEYADETATIVEFLNRWITFCQSIISTLT |
| 160 | hIL-2 | APTSSSTKKTQLQLEHLLLDLQMILNGINNYKNPKLTRMLTHKFYMPKKATELKHLQCLEEELKPLEEV |
| | F42H/I92D | LNLAQSKNFHLRPRDLISNINVDVLELKGSETTEMCEYADETATIVEFLNRWITFCQSIISTLT |
| 161 | hIL-2 | APTSSSTKKTQLQLEHLLLDLQMILNGINNYKNPKLTRMLTAKFYMPKKATELKHLQCLEEELKPLEEV |
| | F42A/I92D | LNLAQSKNFHLRPRDLISNINVDVLELKGSETTEMCEYADETATIVEFLNRWITFCQSIISTLT |
| 162 | hIL-2 | APTSSSTKKTQLQLEHLLLDLQMILNGINNYKNPKLTRMLTFEFYMPKKATELKHLQCLEEELKPLEEV |
| | K43E/I92D | LNLAQSKNFHLRPRDLISNINVDVLELKGSETTFMCEYADETATIVEFLNRWITFCQSIISTLT |
| 163 | hIL-2 | APTSSSTKKTQLQLEHLLLDLQMILNGINNYKNPKLTRMLTFKFRMPKKATELKHLQCLEEELKPLEEV |
| | Y45R/I92D | LNLAQSKNFHLRPRDLISNINVDVLELKGSETTFMCEYADETATIVEFLNRWITFCQSIISTLT |
| 164 | hIL-2 Y45K/I92 | APTSSSTKKTQLQLEHLLLDLQMILNGINNYKNPKLTRMLTFKFKMPKKATELKHLQCLEEELKPLEEV |
| | | LNLAQSKNFHLRPRDLISNINVDVLELKGSETTFMCEYADETATIVEFLNRWITFCQSIISTLT |
| 165 | hIL-2 | APTSSSTKKTQLQLEHLLLDLQMILNGINNYKNPKLTRMLTFKFYMPKKATELKHLQCLEEALKPLEEV |
| | E62A/I92D | LNLAQSKNFHLRPRDLISNINVDVLELKGSETTFMCEYADETATIVEFLNRWITFCQSIISTLT |
| 166 | hIL-2 | APTSSSTKKTQLQLEHLLLDLQMILNGINNYKNPKLTRMLTFKFYMPKKATELKHLQCLEEYLKPLEEV |
| | E62Y/I92D | LNLAQSKNFHLRPRDLISNINVDVLELKGSETTEMCEYADETATIVEFLNRWITFCQSIISTLT |
| 167 | hIL-2 | APTSSSTKKTQLQLEHLLLDLQMILNGINNYKNPKLTRMLTFKFYMPKKATELKHLQCLEEELKPLEEV |
| | L72D/I92D | LNDAQSKNFHLRPRDLISNINVDVLELKGSETTEMCEYADETATIVEFLNRWITFCQSIISTLT |
| 168 | hIL-2 | APTSSSTKKTQLQLEHLLLDLQMILNGINNYKNPKLTRMLTFKFYMPKKATELKHLQCLEEELKPLEEV |
| | L72H/I92D | LNHAQSKNFHLRPRDLISNINVDVLELKGSETTFMCEYADETATIVEFLNRWITFCQSIISTLT |
| 169 | hIL-2 | APTSSSTKKTQLQLEHLLLDLQMILNGINNYKNPKLTRMLTFKFYMPKKATELKHLQCLEEELKPLEEV |
| | L72R/I92D | LNRAQSKNFHLRPRDLISNINVDVLELKGSETTFMCEYADETATIVEFLNRWITFCQSIISTLT |
| 170 | hIL-2 | APTSSSTKKTQLQLEHLLLDLQMILNGINNYKNPKLTDMLTFKFYMPKKATELKHLQCLEEELKPLEEV |
| | R38D/I92D | LNLAQSKNFHLRPRDLISNINVDVLELKGSETTFMCEYADETATIVEFLNRWITFCQSIISTLT |
| 171 | hIL-2 | APTSSSTKKTQLQLEHLLLDLQMILNGINNYKNPKLTEMLTFKFYMPKKATELKHLQCLEEELKPLEEV |
| | R38E/I92D | LNLAQSKNFHLRPRDLISNINVDVLELKGSETTEMCEYADETATIVEFLNRWITFCQSIISTLT |
| 172 | hIL-2 | APTSSSTKKTQLQLEHLLLDLQMILNGINNYKNPKLTQMLTFKFYMPKKATELKHLQCLEEELKPLEEV |
| | R38Q/I92D | LNLAQSKNFHLRPRDLISNINVDVLELKGSETTFMCEYADETATIVEFLNRWITFCQSIISTLT |
| 173 | hIL-2 | APTSSSTKKTQLQLEHLLLDLQMILNGINNYKNPKLTAMLTFKFYMPKKATELKHLQCLEEELKPLEEV |
| | R38A/I92D | LNLAQSKNFHLRPRDLISNINVDVLELKGSETTEMCEYADETATIVEFLNRWITFCQSIISTLT |
| 174 | hIL-2 | APTSSSTKKTQLQLEHLLLDLQMILNGINNYKNPKLTEMLTFKFYMPKKATELKHLQCLEEELKPLEEV |
| | R38E/N88R | LNLAQSKNFHLRPRDLISRINVIVLELKGSETTFMCEYADETATIVEFLNRWITFCQSIISTLT |
| 175 | hIL-2 | APTSSSTKKTQLQLEHLLLDLQMILNGINNYKNPKLTEMLTFKFYMPKKATELKHLQCLEEELKPLEEV |
| | R38E/D84R | LNLAQSKNFHLRPRRLISNINVIVLELKGSETTEMCEYADETATIVEFLNRWITFCQSIISTLT |
| 176 | hIL-2 | APTSSSTKKTQLQLEHLLLDLQMILNGINNYKNPKLTEMLTFKFYMPKKATELKHLQCLEEELKPLEEV |
| | R38E/D84K | LNLAQSKNFHLRPRKLISNINVIVLELKGSETTFMCEYADETATIVEFLNRWITFCQSIISTLT |
| 177 | hIL-2 | APTSSSTKKTQLQLEHLLLALQMILNGINNYKNPKLTRMLTAKFRMPKKATELKHLQCLEEELKPLEEV |
| | F42A/Y45R/D20A | LNLAQSKNFHLRPRDLISNINVIVLELKGSETTFMCEYADETATIVEFLNRWITFCQSIISTLT |
| 178 | hIL-2 | APTSSSTKKTQLQLEHLLLALQMILNGINNYKNPKLTRMLTHKFRMPKKATELKHLOCLEEELKPLEEV |
| | F42H/Y45R/D20A | LNLAQSKNFHLRPRDLISNINVIVLELKGSETTEMCEYADETATIVEFLNRWITFCQSIISTLT |
| 179 | hIL-2 | APTSSSTKKTQLQLEHLLLALQMILNGINNYKNPKLTDMLTFKFYMPKKATELKHLQCLERELKPLEEV |
| | R38D/E61R/D20A | LNLAQSKNFHLRPRDLISNINVIVLELKGSETTFMCEYADETATIVEFLNRWITFCQSIISTLT |
| 180 | hIL-2 | APTSSSTKKTQLQLEHLLLALQMILNGINNYKNPKLTEMLTFKFYMPKKATELKHLQCLERELKPLEEV |
| | R38E/E61R/D20A | LNLAQSKNFHLRPRDLISNINVIVLELKGSETTFMCEYADETATIVEFLNRWITFCQSIISTLT |
| 181 | hIL-2 | APTSSSTKKTQLQLEHLLLALQMILNGINNYKNPKLTQMLTFKFYMPKKATELKHLQCLERELKPLEEV |
| | R38Q/E61R/D20A | LNLAQSKNFHLRPRDLISNINVIVLELKGSETTFMCEYADETATIVEFLNRWITFCQSIISTLT |
| 182 | hIL-2 | APTSSSTKKTQLQLEHLLLALQMILNGINNYKNPKLTAMLTFKFYMPKKATELKHLQCLERELKPLEEV |
| | R38A/E61R/D20A | LNLAQSKNFHLRPRDLISNINVIVLELKGSETTFMCEYADETATIVEFLNRWITFCQSIISTLT |
| 183 | hIL-2 | APTSSSTKKTQLQLEHLLLALQMILNGINNYKNPKLTAMLTFKFYMPKKATELKHLQCLEEELKPLEEV |
| | R38A/D20A/E95A | LNLAQSKNFHLRPRDLISNINVIVLALKGSETTFMCEYADETATIVEFLNRWITFCQSIISTLT |
| 184 | hIL-2 | APTSSSTKKTQLQLEHLLLALQMILNGINNYKNPKLTDMLTFKFYMPKKATELKHLQCLEEELKPLEEV |
| | D20A/E95A/R38D | LNLAQSKNFHLRPRDLISNINVIVLALKGSETTFMCEYADETATIVEFLNRWITFCQSIISTLT |
| 185 | hIL-2 D20A/ | APTSSSTKKTQLQLEHLLLALQMILNGINNYKNPKLTEMLTFKFYMPKKATELKHLQCLEEELKPLEEV |
| | E95A/ R38E | LNLAQSKNFHLRPRDLISNINVIVLALKGSETTFMCEYADETATIVEFLNRWITFCQSIISTLT |
| 186 | hIL-2 | APTSSSTKKTQLQLEHLLLALQMILNGINNYKNPKLTQMLTFKFYMPKKATELKHLQCLEEELKPLEEV |
| | D20A/E95A/R38Q | LNLAQSKNFHLRPRDLISNINVIVLALKGSETTEMCEYADETATIVEFLNRWITFCQSIISTLT |
| 187 | hIL-2 | APTSSSTKKTQLQLEHLLLALQMILNGINNYKNPKLTRMLTRKFYMPKKATELKHLQCLEEELKPLEEV |
| | D20A/E95A/F42R | LNLAQSKNFHLRPRDLISNINVIVLALKGSETTFMCEYADETATIVEFLNRWITFCQSIISTLT |
| 188 | hIL-2 | APTSSSTKKTQLQLEHLLLALQMILNGINNYKNPKLTRMLTAKFYMPKKATELKHLQCLEEELKPLEEV |
| | D20A/E95A/F42A | LNLAQSKNFHLRPRDLISNINVIVLALKGSETTFMCEYADETATIVEFLNRWITFCQSIISTLT |
| 189 | hIL-2D20A/ | APTSSSTKKTQLQLEHLLLALQMILNGINNYKNPKLTRMLTDKFYMPKKATELKHLQCLEEELKPLEEV |
| | E95A/ | LNLAQSKNFHLRPRDLISNINVIVLALKGSETTFMCEYADETATIVEFLNRWITFCQSIISTLT |
| | F42D | |
| 190 | hIL-2 | APTSSSTKKTQLQLEHLLLALQMILNGINNYKNPKLTRMLTHKFYMPKKATELKHLQCLEEELKPLEEV |
| | D20A/E95A/ | LNLAQSKNFHLRPRDLISNINVIVLALKGSETTEMCEYADETATIVEFLNRWITFCQSIISTLT |
| | F42H | |
| 191 | hIL-2 | APTSSSTKKTQLQLEHLLLALQMILNGINNYKNPKLTRMLTKKFYMPKKATELKHLQCLEEELKPLEEV |
| | D20A/E95A/F42K | LNLAQSKNFHLRPRDLISNINVIVLALKGSETTFMCEYADETATIVEFLNRWITFCQSIISTLT |
| 192 | hIL-2 | APTSSSTKKTQLQLEHLLLALQMILNGINNYKNPKLTRMLTFAFYMPKKATELKHLQCLEEELKPLEEV |
| | D20A/E95A/ | LNLAQSKNFHLRPRDLISNINVIVLALKGSETTFMCEYADETATIVEFLNRWITFCQSIISTLT |
| | K43A | |
| 193 | hIL-2 | APTSSSTKKTQLQLEHLLLALQMILNGINNYKNPKLTRMLTFEFYMPKKATELKHLQCLEEELKPLEEV |
| | D20A/E95A/ | LNLAQSKNFHLRPRDLISNINVIVLALKGSETTFMCEYADETATIVEFLNRWITFCQSIISTLT |
| | K43E | |
| 194 | hIL-2 | APTSSSTKKTQLQLEHLLLALQMILNGINNYKNPKLTRMLTFQFYMPKKATELKHLQCLEEELKPLEEV |
| | D20A/E95A/ | LNLAQSKNFHLRPRDLISNINVIVLALKGSETTFMCEYADETATIVEFLNRWITFCQSIISTLT |
| | K43Q | |
| 195 | hIL-2 | APTSSSTKKTQLQLEHLLLALQMILNGINNYKNPKLTRMLTFKFAMPKKATELKHLQCLEEELKPLEEV |
| | D20A/E95A/ | LNLAQSKNFHLRPRDLISNINVIVLALKGSETTFMCEYADETATIVEFLNRWITFCQSIISTLT |
| | Y45A | |
| 196 | hIL-2 | APTSSSTKKTQLQLEHLLLALQMILNGINNYKNPKLTRMLTFKFKMPKKATELKHLQCLEEELKPLEEV |
| | D20A/E95A/ | LNLAQSKNFHLRPRDLISNINVIVLALKGSETTFMCEYADETATIVEFLNRWITFCQSIISTLT |
| | Y45K | |
| 197 | hIL-2 | APTSSSTKKTQLQLEHLLLALQMILNGINNYKNPKLTRMLTFKESMPKKATELKHLQCLEEELKPLEEV |
| | D20A/E95A/ | LNLAQSKNFHLRPRDLISNINVIVLALKGSETTFMCEYADETATIVEFLNRWITFCQSIISTLT |
| | Y45S | |
| 198 | hIL-2 | APTSSSTKKTQLQLEHLLLALQMILNGINNYKNPKLTRMLTFKFRMPKKATELKHLQCLEEELKPLEEV |
| | D20A/E95A/ | LNLAQSKNFHLRPRDLISNINVIVLALKGSETTEMCEYADETATIVEFLNRWITFCQSIISTLT |
| | Y45R | |
| 199 | hIL-2 | APTSSSTKKTQLQLEHLLLALQMILNGINNYKNPKLTRMLTFKFYMPKKATELKHLQCLEAELKPLEEV |
| | D20A/E95A/ | LNLAQSKNFHLRPRDLISNINVIVLALKGSETTFMCEYADETATIVEFLNRWITFCQSIISTLT |
| | E61A | |
| 200 | hIL-2 | APTSSSTKKTQLQLEHLLLALQMILNGINNYKNPKLTRMLTFKFYMPKKATELKHLQCLEEALKPLEEV |
| | D20A/E95A/ | LNLAQSKNFHLRPRDLISNINVIVLALKGSETTFMCEYADETATIVEFLNRWITFCQSIISTLT |
| | E62A | |
| 201 | hIL-2 | APTSSSTKKTQLQLEHLLLALQMILNGINNYKNPKLTRMLTFKFYMPKKATELKHLQCLEERLKPLEEV |
| | D20A/E95A/E62R | LNLAQSKNFHLRPRDLISNINVIVLALKGSETTFMCEYADETATIVEFLNRWITFCQSIISTLT |
| 202 | hIL-2 | APTSSSTKKTQLQLEHLLLALQMILNGINNYKNPKLTRMLTFKFYMPKKATELKHLQCLEEKLKPLEEV |
| | D20A/E95A/ | LNLAQSKNFHLRPRDLISNINVIVLALKGSETTFMCEYADETATIVEFLNRWITFCQSIISTLT |
| | E62K | |
| 203 | hIL-2 | APTSSSTKKTQLQLEHLLLALQMILNGINNYKNPKLTRMLTFKFYMPKKATELKHLQCLEEYLKPLEEV |
| | D20A/E95A/ | LNLAQSKNFHLRPRDLISNINVIVLALKGSETTFMCEYADETATIVEFLNRWITFCQSIISTLT |
| | E62Y | |
| 204 | hIL-2 | APTSSSTKKTQLQLEHLLLALQMILNGINNYKNPKLTRMLTFKFYMPKKATELKHLQCLEEELKPLEYV |
| | D20A/E95A/ | LNLAQSKNFHLRPRDLISNINVIVLALKGSETTFMCEYADETATIVEFLNRWITFCQSIISTLT |
| | E68Y | |
| 205 | hIL-2 | APTSSSTKKTQLQLEHLLLALQMILNGINNYKNPKLTRMLTFKFYMPKKATELKHLQCLEEELKPLEAV |
| | D20A/E95A/E68A | LNLAQSKNFHLRPRDLISNINVIVLALKGSETTEMCEYADETATIVEFLNRWITFCQSIISTLT |
| 206 | hIL-2 | APTSSSTKKTQLQLEHLLLALQMILNGINNYKNPKLTRMLTFKFYMPKKATELKHLQCLEEELKPLELV |
| | D20A/E95A/ | LNLAQSKNFHLRPRDLISNINVIVLALKGSETTFMCEYADETATIVEFLNRWITFCQSIISTLT |
| | E68L | |
| 207 | hIL-2 | APTSSSTKKTQLQLEHLLLALQMILNGINNYKNPKLTRMLTFKFYMPKKATELKHLQCLEEELKPLEEV |
| | D20A/E95A/ | LNYAQSKNFHLRPRDLISNINVIVLALKGSETTFMCEYADETATIVEFLNRWITFCQSIISTLT |
| | L72Y | |
| 208 | hIL-2 | APTSSSTKKTQLQLEHLLLALQMILNGINNYKNPKLTRMLTFKFYMPKKATELKHLQCLEEELKPLEEV |
| | D20A/E95A/ | LNRAQSKNFHLRPRDLISNINVIVLALKGSETTFMCEYADETATIVEFLNRWITFCQSIISTLT |
| | L72R | |
| 209 | hIL-2 | APTSSSTKKTQLQLEHLLLALQMILNGINNYKNPKLTRMLTFKFYMPKKATELKHLQCLEEELKPLEEV |
| | D20A/E95A/ | LNAAQSKNFHLRPRDLISNINVIVLALKGSETTFMCEYADETATIVEFLNRWITFCQSIISTLT |
| | L72A | |
| 210 | hIL-2 | APTSSSTKKTQLQLEHLLLALQMILNGINNYKNPKLTRMLTFKFYMPKKATELKHLQCLEEELKPLEEV |
| | D20A/E95A/L72D | LNDAQSKNFHLRPRDLISNINVIVLALKGSETTFMCEYADETATIVEFLNRWITFCQSIISTLT |
| 211 | hIL-2 | APTSSSTKKTQLQLEHLLLALQMILNGINNYKNPKLTRMLTFKFYMPKKATELKHLQCLEEELKPLEEV |
| | D20A/E95A/ | LNHAQSKNFHLRPRDLISNINVIVLALKGSETTFMCEYADETATIVEFLNRWITFCQSIISTLT |
| | L72H | |
| 212 | hIL-2 | APTSSSTKKTQLQLEHLLLALQMILNGINNYKNPKLTRMLTFKFYMPKKATELKHLQCLEEELKPLEEV |
| | D20A/E95A/ | LNFAQSKNFHLRPRDLISNINVIVLALKGSETTFMCEYADETATIVEFLNRWITFCQSIISTLT |
| | L72F | |
| 213 | hIL-2 | APTSSSTKKTQLQLEHLLLALQMILNGINNYKNPKLTRMLTKKFRMPKKATELKHLQCLEEELKPLEEV |
| | F42K/Y45R/D20A/ | LNLAQSKNFHLRPRDLIANINVIVLELKGSETTEMCEYADETATIVE FLNRWITFCQSIISTLT |
| | S87A | |
| 214 | hIL-2 | APTSSSTKKTQLQLEHLLLALQMILNGINNYKNPKLTRMLTKKFRMPKKATELKHLQCLEEELKPLEEV |
| | F42K/Y45R/D20A/ | LNLAQSKNFHLRPRDLISNINVIVLALKGSETTEMCEYADETATIVEFLNRWITFCQSIISTLT |
| | E95A | |
| 215 | hIL-2 | APTSSSTKKTQLQLEHLLLALQMILNGINNYKNPKLTEMLTFKFYMPKKATELKHLQCLEEELKPLEEV |
| | D20A/R38E/C125A | LNLAQSKNFHLRPRDLISNINVIVLELKGSETTEMCEYADETATIVEFLNRWITFAQSIISTLT |
| 216 | hIL-2 | APASSSTKKTQLQLEHLLLALQMILNGINNYKNPKLTEMLTFKFYMPKKATELKHLQCLEEELKPLEEV |
| | T3A/D20A/R38E | LNLAQSKNFHLRPRDLISNINVIVLELKGSETTFMCEYADETATIVEFLNRWITFCQSIISTLT |
| 217 | hIL-2 | APASSSTKKTQLQLEHLLLALQMILNGINNYKNPKLTEMLTFKFYMPKKATELKHLQCLEEELKPLEEV |
| | T3A/D20A/R38E/ | LNLAQSKNFHLRPRDLISNINVIVLELKGSETTEMCEYADETATIVEFLNRWITFAQSIISTLT |
| | C125A | |
| | (IL-2-AAEA) | |
| 218 | hIL-2 A1- | SSSTKKTQLQLEHLLLALQMILNGINNYKNPKLTEMLTFKFYMPKKATELKHLQCLEEELKPLEEVLNL |
| | 3APT/D20A/R38E | AQSKNFHLRPRDLISNINVIVLELKGSETTEMCEYADETATIVEFLNRWITFCQSIISTLT |
| 219 | hIL-2 A1- | SSSTKKTQLQLEHLLLALQMILNGINNYKNPKLTEMLTFKFYMPKKATELKHLQCLEEELKPLEEVLNL |
| | 3APT/D20A/R38E/ | AQSKNFHLRPRDLISNINVIVLELKGSETTEMCEYADETATIVEFLNRWITFAQSIISTLT |
| | C125A | |
| 220 | hIL-2 | APTSSSTKKTQLQLEHLLLDLAMILNGINNYKNPKLTEMLTFKFYMPKKATELKHLQCLEEELKPLEEV |
| | R38E/Q22A | LNLAQSKNFHLRPRDLISNINVIVLELKGSETTFMCEYADETATIVEFLNRWITFCQSIISTLT |
| 221 | hIL-2 | APTSSSTKKTQLQLEHLLLDLQMILNGINNYKNPKLTEMLTFKFYMPKKATELKHLQCLEEELKPLEEV |
| | R38E/T123A | LNLAQSKNFHLRPRDLISNINVIVLELKGSETTEMCEYADETATIVEFLNRWIAFCQSIISTLT |
| 222 | hIL-2 | APTSSSTKKTQLQLEHLLLDLQMILNGINNYKNPKLTEMLTFKFYMPKKATELKHLQCLEEELKPLEEV |
| | R38E/I129A | LNLAQSKNFHLRPRDLISNINVIVLELKGSETTEMCEYADETATIVEFLNRWITFCQSIASTLT |
| 223 | hIL-2 | APTSSSTKKTQLQLEHLLLDLQMILNGINNYKNPKLTEMLTFKFYMPKKATELKHLQCLEEELKPLEEV |
| | R38E/S130A | LNLAQSKNFHLRPRDLISNINVIVLELKGSETTEMCEYADETATIVEFLNRWITFCQSIIATLT |
| 224 | hIL-2 | APTSSSTKKTQLQLEHLLLDLQMILNGINNYKNPKLTEMLTFKFYMPKKATELKHLQCLEEELKPLEEV |
| | R38E/Q126A | LNLAQSKNFHLRPRDLISNINVIVLELKGSETTFMCEYADETATIVEFLNRWITFCASIISTLT |
| 225 | hIL-2 | APTSSSTKKTQLQLEHLLLDLQMILNGINNYKNPKLTEMLTFKFYMPKKATELKHLQCLEEELKPLEEV |
| | R38E/Q126D | LNLAQSKNFHLRPRDLISNINVIVLELKGSETTFMCEYADETATIVEFLNRWITFCDSIISTLT |
| 226 | hIL-2 | APTSSSTKKTQLQLEHLLLDLQMILNGINNYKNPKLTEMLTFKFYMPKKATELKHLQCLEEELKPLEEV |
| | R38E/Q126V | LNLAQSKNFHLRPRDLISNINVIVLELKGSETTFMCEYADETATIVEFLNRWITFCVSIISTLT |
| 227 | hIL-2 | APTSSSTKKTQLQLEHLLLDLAMILNGINNYKNPKLTEMLTFKFYMPKKATELKHLQCLEEELKPLEEV |
| | R38E/Q22A/S130A | LNLAQSKNFHLRPRDLISNINVIVLELKGSETTFMCEYADETATIVEFLNRWITFCQSIIATLT |
| 228 | hIL-2 | APTSSSTKKTQLQLEHLLLDLQMILNGINNYKNPKLTRMLTKKFRMPKKATELKHLQCLEEELKPLEEV |
| | F42K/Y45R/Q126D | LNLAQSKNFHLRPRDLISNINVIVLELKGSETTEMCEYADETATIVEFLNRWITFCDSIISTLT |
| 229 | hIL-2 | APTSSSTKKTQLQLEHLLLALQMILNGINNYKNPKLTRMLTFKFYMPKKATELKHLQCLEEELKPLEEV |
| | D20A/E95A/Q126D | LNLAQSKNFHLRPRDLISNINVIVLALKGSETTFMCEYADETATIVEFLNRWITFCDSIISTLT |
| 230 | hIL-2 | APTSSSTKKTQLQLEHLLLALQMILNGINNYKNPKLTRMLTFKFYMPKKATELKHLQCLERELKPLEEV |
| | D20A/E61R | LNLAQSKNFHLRPRDLISNINVIVLELKGSETTFMCEYADETATIVEFLNRWITFCQSIISTLT |
| 231 | hIL-2 | APTSSSTKKTQLQLEHLLLALQMILNGINNYKNPKLTRMLTFKFYMPKKATELKHLQCLENELKPLEEV |
| | D20A/E61N | LNLAQSKNFHLRPRDLISNINVIVLELKGSETTFMCEYADETATIVEFLNRWITFCQSIISTLT |
| 232 | hIL-2 | APTSSSTKKTQLQLEHLLLALQMILNGINNYKNPKLTRMLTFKFYMPKKATELKHLQCLEDELKPLEEV |
| | D20A/E61D | LNLAQSKNFHLRPRDLISNINVIVLELKGSETTEMCEYADETATIVEFLNRWITFCQSIISTLT |
| 233 | hIL-2 | APTSSSTKKTQLQLEHLLLALQMILNGINNYKNPKLTRMLTFKFYMPKKATELKHLQCLEQELKPLEEV |
| | D20A/E61Q | LNLAQSKNFHLRPRDLISNINVIVLELKGSETTFMCEYADETATIVEFLNRWITFCQSIISTLT |
| 234 | hIL-2 | APTSSSTKKTQLQLEHLLLALQMILNGINNYKNPKLTRMLTFKFYMPKKATELKHLQCLEGELKPLEEV |
| | D20A/E61G | LNLAQSKNFHLRPRDLISNINVIVLELKGSETTEMCEYADETATIVEFLNRWITFCQSIISTLT |
| 235 | hIL-2 | APTSSSTKKTQLQLEHLLLALQMILNGINNYKNPKLTRMLTFKFYMPKKATELKHLQCLEHELKPLEEV |
| | D20A/E61H | LNLAQSKNFHLRPRDLISNINVIVLELKGSETTFMCEYADETATIVEFLNRWITFCQSIISTLT |
| 236 | hIL-2 | APTSSSTKKTQLQLEHLLLALQMILNGINNYKNPKLTRMLTFKFYMPKKATELKHLQCLEIELKPLEEV |
| | D20A/E61I | LNLAQSKNFHLRPRDLISNINVIVLELKGSETTFMCEYADETATIVEFLNRWITFCQSIISTLT |
| 237 | hIL-2 | APTSSSTKKTQLQLEHLLLALQMILNGINNYKNPKLTRMLTFKFYMPKKATELKHLQCLELELKPLEEV |
| | D20A/E61L | LNLAQSKNFHLRPRDLISNINVIVLELKGSETTFMCEYADETATIVEFLNRWITFCQSIISTLT |
| 238 | hIL-2 | APTSSSTKKTQLQLEHLLLALQMILNGINNYKNPKLTRMLTFKFYMPKKATELKHLQCLEKELKPLEEV |
| | D20A/E61K | LNLAQSKNFHLRPRDLISNINVIVLELKGSETTEMCEYADETATIVEFLNRWITFCQSIISTLT |
| 239 | hIL-2 | APTSSSTKKTQLQLEHLLLALQMILNGINNYKNPKLTRMLTFKFYMPKKATELKHLQCLEMELKPLEEV |
| | D20A/E61M | LNLAQSKNFHLRPRDLISNINVIVLELKGSETTFMCEYADETATIVEFLNRWITFCQSIISTLT |
| 240 | hIL-2 | APTSSSTKKTQLQLEHLLLALQMILNGINNYKNPKLTRMLTFKFYMPKKATELKHLQCLEFELKPLEEV |
| | D20A/E61F | LNLAQSKNFHLRPRDLISNINVIVLELKGSETTEMCEYADETATIVEFLNRWITFCQSIISTLT |
| 241 | hIL-2 | APTSSSTKKTQLQLEHLLLALQMILNGINNYKNPKLTRMLTFKFYMPKKATELKHLQCLEPELKPLEEV |
| | D20A/E61P | LNLAQSKNFHLRPRDLISNINVIVLELKGSETTEMCEYADETATIVEFLNRWITFCQSIISTLT |
| 242 | hIL-2 | APTSSSTKKTQLQLEHLLLALQMILNGINNYKNPKLTRMLTFKFYMPKKATELKHLQCLESELKPLEEV |
| | D20A/E61S | LNLAQSKNFHLRPRDLISNINVIVLELKGSETTFMCEYADETATIVEFLNRWITFCQSIISTLT |
| 243 | hIL-2 | APTSSSTKKTQLQLEHLLLALQMILNGINNYKNPKLTRMLTFKFYMPKKATELKHLQCLETELKPLEEV |
| | D20A/E61T | LNLAQSKNFHLRPRDLISNINVIVLELKGSETTFMCEYADETATIVEFLNRWITFCQSIISTLT |
| 244 | hIL-2 | APTSSSTKKTQLQLEHLLLALQMILNGINNYKNPKLTRMLTFKFYMPKKATELKHLQCLEWELKPLEEV |
| | D20A/E61W | LNLAQSKNFHLRPRDLISNINVIVLELKGSETTFMCEYADETATIVEFLNRWITFCQSIISTLT |
| 245 | hIL-2 | APTSSSTKKTQLQLEHLLLALQMILNGINNYKNPKLTRMLTFKFYMPKKATELKHLQCLEYELKPLEEV |
| | D20A/E61Y | LNLAQSKNFHLRPRDLISNINVIVLELKGSETTFMCEYADETATIVEFLNRWITFCQSIISTLT |
| 246 | hIL-2 | APTSSSTKKTQLQLEHLLLALQMILNGINNYKNPKLTRMLTFKFYMPKKATELKHLQCLEVELKPLEEV |
| | D20A/E61V | LNLAQSKNFHLRPRDLISNINVIVLELKGSETTEMCEYADETATIVEFLNRWITFCQSIISTLT |
| 247 | hIL-2 | APTSSSTKKTQLQLEHLLLALQMILNGINNYKNPKLTRMLTNKFYMPKKATELKHLQCLEEELKPLEEV |
| | D20A/F42N | LNLAQSKNFHLRPRDLISNINVIVLELKGSETTEMCEYADETATIVEFLNRWITFCQSIISTLT |
| 248 | hIL-2 | APTSSSTKKTQLQLEHLLLALQMILNGINNYKNPKLTRMLTQKFYMPKKATELKHLQCLEEELKPLEEV |
| | D20A/F42Q | LNLAQSKNFHLRPRDLISNINVIVLELKGSETTFMCEYADETATIVEFLNRWITFCQSIISTLT |
| 249 | hIL-2 | APTSSSTKKTQLQLEHLLLALQMILNGINNYKNPKLTRMLTEKFYMPKKATELKHLQCLEEELKPLEEV |
| | D20A/F42E | LNLAQSKNFHLRPRDLISNINVIVLELKGSETTFMCEYADETATIVEFLNRWITFCQSIISTLT |
| 250 | hIL-2 | APTSSSTKKTQLQLEHLLLALQMILNGINNYKNPKLTRMLTGKFYMPKKATELKHLQCLEEELKPLEEV |
| | D20A/F42G | LNLAQSKNFHLRPRDLISNINVIVLELKGSETTFMCEYADETATIVEFLNRWITFCQSIISTLT |
| 251 | hIL-2 | APTSSSTKKTQLQLEHLLLALQMILNGINNYKNPKLTRMLTIKFYMPKKATELKHLQCLEEELKPLEEV |
| | D20A/F42I | LNLAQSKNFHLRPRDLISNINVIVLELKGSETTFMCEYADETATIVEFLNRWITFCQSIISTLT |
| 252 | hIL-2 | APTSSSTKKTQLQLEHLLLALQMILNGINNYKNPKLTRMLTLKFYMPKKATELKHLQCLEEELKPLEEV |
| | D20A/F42L | LNLAQSKNFHLRPRDLISNINVIVLELKGSETTEMCEYADETATIVEFLNRWITFCQSIISTLT |
| 253 | hIL-2 | APTSSSTKKTQLQLEHLLLALQMILNGINNYKNPKLTRMLTMKFYMPKKATELKHLQCLEEELKPLEEV |
| | D20A/F42M | LNLAQSKNFHLRPRDLISNINVIVLELKGSETTEMCEYADETATIVEFLNRWITFCQSIISTLT |
| 254 | hIL-2 | APTSSSTKKTQLQLEHLLLALQMILNGINNYKNPKLTRMLTPKFYMPKKATELKHLQCLEEELKPLEEV |
| | D20A/F42P | LNLAQSKNFHLRPRDLISNINVIVLELKGSETTEMCEYADETATIVEFLNRWITFCQSIISTLT |
| 255 | hIL-2 | APTSSSTKKTQLQLEHLLLALQMILNGINNYKNPKLTRMLTSKFYMPKKATELKHLQCLEEELKPLEEV |
| | D20A/F42S | LNLAQSKNFHLRPRDLISNINVIVLELKGSETTEMCEYADETATIVEFLNRWITFCQSIISTLT |
| 256 | hIL-2 | APTSSSTKKTQLQLEHLLLALQMILNGINNYKNPKLTRMLTTKFYMPKKATELKHLQCLEEELKPLEEV |
| | D20A/F42T | LNLAQSKNFHLRPRDLISNINVIVLELKGSETTFMCEYADETATIVEFLNRWITFCQSIISTLT |
| 257 | hIL-2 | APTSSSTKKTQLQLEHLLLALQMILNGINNYKNPKLTRMLTWKFYMPKKATELKHLQCLEEELKPLEEV |
| | D20A/F42W | LNLAQSKNFHLRPRDLISNINVIVLELKGSETTFMCEYADETATIVEFLNRWITFCQSIISTLT |
| 258 | hIL-2 | APTSSSTKKTQLQLEHLLLALQMILNGINNYKNPKLTRMLTYKFYMPKKATELKHLQCLEEELKPLEEV |
| | D20A/F42Y | LNLAQSKNFHLRPRDLISNINVIVLELKGSETTFMCEYADETATIVEFLNRWITFCQSIISTLT |
| 259 | hIL-2 | APTSSSTKKTQLQLEHLLLALQMILNGINNYKNPKLTRMLTVKFYMPKKATELKHLQCLEEELKPLEEV |
| | D20A/F42V | LNLAQSKNFHLRPRDLISNINVIVLELKGSETTEMCEYADETATIVEFLNRWITFCQSIISTLT |
| 260 | hIL-2 | APTSSSTKKTQLQLEHLLLALQMILNGINNYKNPKLTRMLTFKFAMPKKATELKHLQCLEEELKPLEEV |
| | D20A/Y45A | LNLAQSKNFHLRPRDLISNINVIVLELKGSETTFMCEYADETATIVEFLNRWITFCQSIISTLT |
| 261 | hIL-2 | APTSSSTKKTQLQLEHLLLALQMILNGINNYKNPKLTRMLTFKFNMPKKATELKHLQCLEEELKPLEEV |
| | D20A/Y45N | LNLAQSKNFHLRPRDLISNINVIVLELKGSETTEMCEYADETATIVEFLNRWITFCQSIISTLT |
| 262 | hIL-2 | APTSSSTKKTQLQLEHLLLALQMILNGINNYKNPKLTRMLTFKFDMPKKATELKHLQCLEEELKPLEEV |
| | D20A/Y45D | LNLAQSKNFHLRPRDLISNINVIVLELKGSETTFMCEYADETATIVEFLNRWITFCQSIISTLT |
| 263 | hIL-2 | APTSSSTKKTQLQLEHLLLALQMILNGINNYKNPKLTRMLTFKFQMPKKATELKHLQCLEEELKPLEEV |
| | D20A/Y45Q | LNLAQSKNFHLRPRDLISNINVIVLELKGSETTEMCEYADETATIVEFLNRWITFCQSIISTLT |
| 264 | hIL-2 | APTSSSTKKTQLQLEHLLLALQMILNGINNYKNPKLTRMLTFKFEMPKKATELKHLQCLEEELKPLEEV |
| | D20A/Y45E | LNLAQSKNFHLRPRDLISNINVIVLELKGSETTFMCEYADETATIVEFLNRWITFCQSIISTLT |
| 265 | hIL-2 | APTSSSTKKTQLQLEHLLLALQMILNGINNYKNPKLTRMLTFKFGMPKKATELKHLQCLEEELKPLEEV |
| | D20A/Y45G | LNLAQSKNFHLRPRDLISNINVIVLELKGSETTFMCEYADETATIVEFLNRWITFCQSIISTLT |
| 266 | hIL-2 | APTSSSTKKTQLQLEHLLLALQMILNGINNYKNPKLTRMLTFKFHMPKKATELKHLQCLEEELKPLEEV |
| | D20A/Y45H | LNLAQSKNFHLRPRDLISNINVIVLELKGSETTEMCEYADETATIVEFLNRWITFCQSIISTLT |
| 267 | hIL-2 | APTSSSTKKTQLQLEHLLLALQMILNGINNYKNPKLTRMLTFKFIMPKKATELKHLQCLEEELKPLEEV |
| | D20A/Y45I | LNLAQSKNFHLRPRDLISNINVIVLELKGSETTFMCEYADETATIVEFLNRWITFCQSIISTLT |
| 268 | hIL-2 | APTSSSTKKTQLQLEHLLLALQMILNGINNYKNPKLTRMLTFKFLMPKKATELKHLQCLEEELKPLEEV |
| | D20A/Y45L | LNLAQSKNFHLRPRDLISNINVIVLELKGSETTEMCEYADETATIVEFLNRWITFCQSIISTLT |
| 269 | hIL-2 | APTSSSTKKTQLQLEHLLLALQMILNGINNYKNPKLTRMLTFKFMMPKKATELKHLQCLEEELKPLEEV |
| | D20A/Y45M | LNLAQSKNFHLRPRDLISNINVIVLELKGSETTEMCEYADETATIVEFLNRWITFCQSIISTLT |
| 270 | hIL-2 | APTSSSTKKTQLQLEHLLLALQMILNGINNYKNPKLTRMLTFKFFMPKKATELKHLQCLEEELKPLEEV |
| | D20A/Y45F | LNLAQSKNFHLRPRDLISNINVIVLELKGSETTFMCEYADETATIVEFLNRWITFCQSIISTLT |
| 271 | hIL-2 | APTSSSTKKTQLQLEHLLLALQMILNGINNYKNPKLTRMLTFKFPMPKKATELKHLQCLEEELKPLEEV |
| | D20A/Y45P | LNLAQSKNFHLRPRDLISNINVIVLELKGSETTEMCEYADETATIVEFLNRWITFCQSIISTLT |
| 272 | hIL-2 | APTSSSTKKTQLQLEHLLLALQMILNGINNYKNPKLTRMLTFKFSMPKKATELKHLQCLEEELKPLEEV |
| | D20A/Y45S | LNLAQSKNFHLRPRDLISNINVIVLELKGSETTFMCEYADETATIVEFLNRWITFCQSIISTLT |
| 273 | hIL-2 | APTSSSTKKTQLQLEHLLLALQMILNGINNYKNPKLTRMLTFKFTMPKKATELKHLQCLEEELKPLEEV |
| | D20A/Y45T | LNLAQSKNFHLRPRDLISNINVIVLELKGSETTFMCEYADETATIVEFLNRWITFCQSIISTLT |
| 274 | hIL-2 | APTSSSTKKTQLQLEHLLLALQMILNGINNYKNPKLTRMLTFKFWMPKKATELKHLQCLEEELKPLEEV |
| | D20A/Y45W | LNLAQSKNFHLRPRDLISNINVIVLELKGSETTEMCEYADETATIVEFLNRWITFCQSIISTLT |
| 275 | hIL-2 | APTSSSTKKTQLQLEHLLLALQMILNGINNYKNPKLTRMLTFKFVMPKKATELKHLQCLEEELKPLEEV |
| | D20A/Y45V | LNLAQSKNFHLRPRDLISNINVIVLELKGSETTFMCEYADETATIVEFLNRWITFCQSIISTLT |
| 276 | hIL-2 | APTSSSTKKTQLQLEHLLLDLQMILNGINNYKNPKLTRMLTNKFYMPKKATELKHLQCLEEELKPLEEV |
| | I92D/F42N | LNLAQSKNFHLRPRDLISNINVDVLELKGSETTFMCEYADETATIVEFLNRWITFCQSIISTLT |
| 277 | hIL-2 | APTSSSTKKTQLQLEHLLLDLQMILNGINNYKNPKLTRMLTQKFYMPKKATELKHLQCLEEELKPLEEV |
| | I92D/F42Q | LNLAQSKNFHLRPRDLISNINVDVLELKGSETTFMCEYADETATIVEFLNRWITFCQSIISTLT |
| 278 | hIL-2 | APTSSSTKKTQLQLEHLLLDLQMILNGINNYKNPKLTRMLTEKFYMPKKATELKHLQCLEEELKPLEEV |
| | I92D/F42E | LNLAQSKNFHLRPRDLISNINVDVLELKGSETTFMCEYADETATIVEFLNRWITFCQSIISTLT |
| 279 | hIL-2 | APTSSSTKKTQLQLEHLLLDLQMILNGINNYKNPKLTRMLTGKFYMPKKATELKHLQCLEEELKPLEEV |
| | I92D/F42G | LNLAQSKNFHLRPRDLISNINVDVLELKGSETTEMCEYADETATIVEFLNRWITFCQSIISTLT |
| 280 | hIL-2 | APTSSSTKKTQLQLEHLLLDLQMILNGINNYKNPKLTRMLTIKFYMPKKATELKHLQCLEEELKPLEEV |
| | I92D/F42I | LNLAQSKNFHLRPRDLISNINVDVLELKGSETTFMCEYADETATIVEFLNRWITFCQSIISTLT |
| 281 | hIL-2 | APTSSSTKKTQLQLEHLLLDLQMILNGINNYKNPKLTRMLTLKFYMPKKATELKHLQCLEEELKPLEEV |
| | I92D/F42L | LNLAQSKNFHLRPRDLISNINVDVLELKGSETTEMCEYADETATIVEFLNRWITFCQSIISTLT |
| 282 | hIL-2 | APTSSSTKKTQLQLEHLLLDLQMILNGINNYKNPKLTRMLTKKFYMPKKATELKHLQCLEEELKPLEEV |
| | I92D/F42K | LNLAQSKNFHLRPRDLISNINVDVLELKGSETTEMCEYADETATIVEFLNRWITFCQSIISTLT |
| 283 | hIL-2 | APTSSSTKKTQLQLEHLLLDLQMILNGINNYKNPKLTRMLTMKFYMPKKATELKHLQCLEEELKPLEEV |
| | I92D/F42M | LNLAQSKNFHLRPRDLISNINVDVLELKGSETTFMCEYADETATIVEFLNRWITFCQSIISTLT |
| 284 | hIL-2 | APTSSSTKKTQLQLEHLLLDLQMILNGINNYKNPKLTRMLTPKFYMPKKATELKHLQCLEEELKPLEEV |
| | I92D/F42P | LNLAQSKNFHLRPRDLISNINVDVLELKGSETTEMCEYADETATIVEFLNRWITFCQSIISTLT |
| 285 | hIL-2 | APTSSSTKKTQLQLEHLLLDLQMILNGINNYKNPKLTRMLTSKFYMPKKATELKHLQCLEEELKPLEEV |
| | I92D/F42S | LNLAQSKNFHLRPRDLISNINVDVLELKGSETTEMCEYADETATIVEFLNRWITFCQSIISTLT |
| 286 | hIL-2 | APTSSSTKKTQLQLEHLLLDLQMILNGINNYKNPKLTRMLTTKFYMPKKATELKHLQCLEEELKPLEEV |
| | I92D/F42T | LNLAQSKNFHLRPRDLISNINVDVLELKGSETTEMCEYADETATIVEFLNRWITFCQSIISTLT |
| 287 | hIL-2 | APTSSSTKKTQLQLEHLLLDLQMILNGINNYKNPKLTRMLTWKFYMPKKATELKHLQCLEEELKPLEEV |
| | I92D/F42W | LNLAQSKNFHLRPRDLISNINVDVLELKGSETTEMCEYADETATIVEFLNRWITFCQSIISTLT |
| 288 | hIL-2 | APTSSSTKKTQLQLEHLLLDLQMILNGINNYKNPKLTRMLTYKFYMPKKATELKHLQCLEEELKPLEEV |
| | I92D/F42Y | LNLAQSKNFHLRPRDLISNINVDVLELKGSETTFMCEYADETATIVEFLNRWITFCQSIISTLT |
| 289 | hIL-2 | APTSSSTKKTQLQLEHLLLDLQMILNGINNYKNPKLTRMLTVKFYMPKKATELKHLQCLEEELKPLEEV |
| | I92D/F42V | LNLAQSKNFHLRPRDLISNINVDVLELKGSETTFMCEYADETATIVEFLNRWITFCQSIISTLT |
| 290 | hIL-2 | APTSSSTKKTQLQLEHLLLDLQMILNGINNYKNPKLTRMLTFKFAMPKKATELKHLQCLEEELKPLEEV |
| | I92D/Y45A | LNLAQSKNFHLRPRDLISNINVDVLELKGSETTEMCEYADETATIVEFLNRWITFCQSIISTLT |
| 291 | hIL-2 | APTSSSTKKTQLQLEHLLLDLQMILNGINNYKNPKLTRMLTFKENMPKKATELKHLQCLEEELKPLEEV |
| | I92D/Y45N | LNLAQSKNFHLRPRDLISNINVDVLELKGSETTEMCEYADETATIVEFLNRWITFCQSIISTLT |
| 292 | hIL-2 | APTSSSTKKTQLQLEHLLLDLQMILNGINNYKNPKLTRMLTFKEDMPKKATELKHLQCLEEELKPLEEV |
| | I92D/Y45D | LNLAQSKNFHLRPRDLISNINVDVLELKGSETTEMCEYADETATIVEFLNRWITFCQSIISTLT |
| 293 | hIL-2 | APTSSSTKKTQLQLEHLLLDLQMILNGINNYKNPKLTRMLTFKFQMPKKATELKHLQCLEEELKPLEEV |
| | I92D/Y45Q | LNLAQSKNFHLRPRDLISNINVDVLELKGSETTEMCEYADETATIVEFLNRWITFCQSIISTLT |
| 294 | hIL-2 | APTSSSTKKTQLQLEHLLLDLQMILNGINNYKNPKLTRMLTFKFEMPKKATELKHLQCLEEELKPLEEV |
| | I92D/Y45E | LNLAQSKNFHLRPRDLISNINVDVLELKGSETTFMCEYADETATIVEFLNRWITFCQSIISTLT |
| 295 | hIL-2 | APTSSSTKKTQLQLEHLLLDLQMILNGINNYKNPKLTRMLTFKFGMPKKATELKHLQCLEEELKPLEEV |
| | I92D/Y45G | LNLAQSKNFHLRPRDLISNINVDVLELKGSETTEMCEYADETATIVEFLNRWITFCQSIISTLT |
| 296 | hIL-2 | APTSSSTKKTQLQLEHLLLDLQMILNGINNYKNPKLTRMLTEKFHMPKKATELKHLQCLEEELKPLEEV |
| | I92D/Y45H | LNLAQSKNFHLRPRDLISNINVDVLELKGSETTEMCEYADETATIVEFLNRWITFCQSIISTLT |
| 297 | hIL-2 | APTSSSTKKTQLQLEHLLLDLQMILNGINNYKNPKLTRMLTEKFIMPKKATELKHLQCLEEELKPLEEV |
| | I92D/Y45I | LNLAQSKNFHLRPRDLISNINVDVLELKGSETTEMCEYADETATIVEFLNRWITFCQSIISTLT |
| 298 | hIL-2 | APTSSSTKKTQLQLEHLLLDLQMILNGINNYKNPKLTRMLTFKFLMPKKATELKHLQCLEEELKPLEEV |
| | I92D/Y45L | LNLAQSKNFHLRPRDLISNINVDVLELKGSETTFMCEYADETATIVEFLNRWITFCQSIISTLT |
| 299 | hIL-2 | APTSSSTKKTQLQLEHLLLDLQMILNGINNYKNPKLTRMLTFKFMMPKKATELKHLQCLEEELKPLEEV |
| | I92D/Y45M | LNLAQSKNFHLRPRDLISNINVDVLELKGSETTFMCEYADETATIVEFLNRWITFCQSIISTLT |
| 300 | hIL-2 | APTSSSTKKTQLQLEHLLLDLQMILNGINNYKNPKLTRMLTFKFFMPKKATELKHLQCLEEELKPLEEV |
| | I92D/Y45F | LNLAQSKNFHLRPRDLISNINVDVLELKGSETTEMCEYADETATIVEFLNRWITFCQSIISTLT |
| 301 | hIL-2 | APTSSSTKKTQLQLEHLLLDLQMILNGINNYKNPKLTRMLTFKFPMPKKATELKHLQCLEEELKPLEEV |
| | I92D/Y45P | LNLAQSKNFHLRPRDLISNINVDVLELKGSETTEMCEYADETATIVEFLNRWITFCQSIISTLT |
| 302 | hIL-2 | APTSSSTKKTQLQLEHLLLDLQMILNGINNYKNPKLTRMLTFKFSMPKKATELKHLQCLEEELKPLEEV |
| | I92D/Y45S | LNLAQSKNFHLRPRDLISNINVDVLELKGSETTFMCEYADETATIVEFLNRWITFCQSIISTLT |
| 303 | hIL-2 | APTSSSTKKTQLQLEHLLLDLQMILNGINNYKNPKLTRMLTFKFTMPKKATELKHLQCLEEELKPLEEV |
| | I92D/Y45T | LNLAQSKNFHLRPRDLISNINVDVLELKGSETTEMCEYADETATIVEFLNRWITFCQSIISTLT |
| 304 | hIL-2 | APTSSSTKKTQLQLEHLLLDLQMILNGINNYKNPKLTRMLTFKFWMPKKATELKHLQCLEEELKPLEEV |
| | I92D/Y45W | LNLAQSKNFHLRPRDLISNINVDVLELKGSETTEMCEYADETATIVEFLNRWITFCQSIISTLT |
| 305 | hIL-2 | APTSSSTKKTQLQLEHLLLDLQMILNGINNYKNPKLTRMLTFKFVMPKKATELKHLQCLEEELKPLEEV |
| | I92D/Y45V | LNLAQSKNFHLRPRDLISNINVDVLELKGSETTEMCEYADETATIVEFLNRWITFCQSIISTLT |
| 306 | hIL-2 | APTSSSTKKTQLQLEHLLLHLQMILNGINNYKNPKLTEMLTFKFYMPKKATELKHLQCLEEELKPLEEV |
| | R38E/D20H | LNLAQSKNFHLRPRDLISNINVIVLELKGSETTFMCEYADETATIVEFLNRWITFCQSIISTLT |
| 307 | hIL-2 | APTSSSTKKTQLQLEHLLLSLQMILNGINNYKNPKLTEMLTFKFYMPKKATELKHLQCLEEELKPLEEV |
| | R38E/D20S | LNLAQSKNFHLRPRDLISNINVIVLELKGSETTFMCEYADETATIVEFLNRWITFCQSIISTLT |
| 308 | hIL-2 | APTSSSTKKTQLQLEHLLLDLQMILNGINNYKNPKLTRMLTAKFYMPKKATELKHLQCLEEELKPLEEV |
| | F42A/N88R | LNLAQSKNFHLRPRDLISRINVIVLELKGSETTEMCEYADETATIVEFLNRWITFCQSIISTLT |
| 309 | hIL-2 | APTSSSTKKTQLQLEHLLLDLQMILNGINNYKNPKLTRMLTAKFYMPKKATELKHLQCLEEELKPLEEV |
| | F42A/N88D | LNLAQSKNFHLRPRDLISDINVIVLELKGSETTFMCEYADETATIVEFLNRWITFCQSIISTLT |
| 310 | hIL-2 | APTSSSTKKTQLQLEHLLLDLQMILNGINNYKNPKLTEMLTFKFYMPKKATELKHLQCLEEELKPLEEV |
| | R38E/D84A | LNLAQSKNFHLRPRALISNINVIVLELKGSETTFMCEYADETATIVEFLNRWITFCQSIISTLT |
| 311 | hIL-2 | APTSSSTKKTQLQLEHLLLDLQMILNGINNYKNPKLTEMLTFKFYMPKKATELKHLOCLEEELKPLEEV |
| | R38E/D84N | LNLAQSKNFHLRPRNLISNINVIVLELKGSETTFMCEYADETATIVEFLNRWITFCQSIISTLT |
| 312 | hIL-2 | APTSSSTKKTQLQLEHLLLDLQMILNGINNYKNPKLTEMLTFKFYMPKKATELKHLQCLEEELKPLEEV |
| | R38E/D84Q | LNLAQSKNFHLRPRQLISNINVIVLELKGSETTEMCEYADETATIVEFLNRWITFCQSIISTLT |
| 313 | hIL-2 | APTSSSTKKTQLQLEHLLLDLQMILNGINNYKNPKLTEMLTFKFYMPKKATELKHLQCLEEELKPLEEV |
| | R38E/D84E | LNLAQSKNFHLRPRELISNINVIVLELKGSETTFMCEYADETATIVEFLNRWITFCQSIISTLT |
| 314 | hIL-2 | APTSSSTKKTQLQLEHLLLDLQMILNGINNYKNPKLTEMLTFKFYMPKKATELKHLQCLEEELKPLEEV |
| | R38E/D84G | LNLAQSKNFHLRPRGLISNINVIVLELKGSETTFMCEYADETATIVEFLNRWITFCQSIISTLT |
| 315 | hIL-2 | APTSSSTKKTQLQLEHLLLDLQMILNGINNYKNPKLTEMLTFKFYMPKKATELKHLQCLEEELKPLEEV |
| | R38E/D84H | LNLAQSKNFHLRPRHLISNINVIVLELKGSETTEMCEYADETATIVEFLNRWITFCQSIISTLT |
| 316 | hIL-2 | APTSSSTKKTQLQLEHLLLDLQMILNGINNYKNPKLTEMLTFKFYMPKKATELKHLQCLEEELKPLEEV |
| | R38E/D84I | LNLAQSKNFHLRPRILISNINVIVLELKGSETTEMCEYADETATIVEFLNRWITFCQSIISTLT |
| 317 | hIL-2 | APTSSSTKKTQLQLEHLLLDLQMILNGINNYKNPKLTEMLTFKFYMPKKATELKHLQCLEEELKPLEEV |
| | R38E/D84L | LNLAQSKNFHLRPRLLISNINVIVLELKGSETTFMCEYADETATIVEFLNRWITFCQSIISTLT |
| 318 | hIL-2 | APTSSSTKKTQLQLEHLLLDLQMILNGINNYKNPKLTEMLTFKFYMPKKATELKHLQCLEEELKPLEEV |
| | R38E/D84M | LNLAQSKNFHLRPRMLISNINVIVLELKGSETTFMCEYADETATIVEFLNRWITFCQSIISTLT |
| 319 | hIL-2 | APTSSSTKKTQLQLEHLLLDLQMILNGINNYKNPKLTEMLTFKFYMPKKATELKHLQCLEEELKPLEEV |
| | R38E/D84F | LNLAQSKNFHLRPRFLISNINVIVLELKGSETTFMCEYADETATIVEFLNRWITFCQSIISTLT |
| 320 | hIL-2 | APTSSSTKKTQLQLEHLLLDLQMILNGINNYKNPKLTEMLTFKFYMPKKATELKHLQCLEEELKPLEEV |
| | R38E/D84P | LNLAQSKNFHLRPRPLISNINVIVLELKGSETTFMCEYADETATIVEFLNRWITFCQSIISTLT |
| 321 | hIL-2 | APTSSSTKKTQLQLEHLLLDLQMILNGINNYKNPKLTEMLTFKFYMPKKATELKHLQCLEEELKPLEEV |
| | R38E/D84S | LNLAQSKNFHLRPRSLISNINVIVLELKGSETTFMCEYADETATIVEFLNRWITFCQSIISTLT |
| 322 | hIL-2 | APTSSSTKKTQLQLEHLLLDLQMILNGINNYKNPKLTEMLTFKFYMPKKATELKHLQCLEEELKPLEEV |
| | R38E/D84T | LNLAQSKNFHLRPRTLISNINVIVLELKGSETTFMCEYADETATIVEFLNRWITFCQSIISTLT |
| 323 | hIL-2 | APTSSSTKKTQLQLEHLLLDLQMILNGINNYKNPKLTEMLTFKFYMPKKATELKHLQCLEEELKPLEEV |
| | R38E/D84W | LNLAQSKNFHLRPRWLISNINVIVLELKGSETTFMCEYADETATIVEFLNRWITFCQSIISTLT |
| 324 | hIL-2 | APTSSSTKKTQLQLEHLLLDLQMILNGINNYKNPKLTEMLTFKFYMPKKATELKHLQCLEEELKPLEEV |
| | R38E/D84Y | LNLAQSKNFHLRPRYLISNINVIVLELKGSETTFMCEYADETATIVEFLNRWITFCQSIISTLT |
| 325 | hIL-2 | APTSSSTKKTQLQLEHLLLDLQMILNGINNYKNPKLTEMLTFKFYMPKKATELKHLQCLEEELKPLEEV |
| | R38E/D84V | LNLAQSKNFHLRPRVLISNINVIVLELKGSETTFMCEYADETATIVEFLNRWITFCQSIISTLT |
| 326 | hIL-2 | APTSSSTKKTQLQLEHLLLDLQMILNGINNYKNPKLTEMLTFKFYMPKKATELKHLQCLEEELKPLEEV |
| | R38E/I92A | LNLAQSKNFHLRPRDLISNINVAVLELKGSETTFMCEYADETATIVEFLNRWITFCQSIISTLT |
| 327 | hIL-2 | APTSSSTKKTQLQLEHLLLDLQMILNGINNYKNPKLTEMLTFKFYMPKKATELKHLQCLEEELKPLEEV |
| | R38E/I92R | LNLAQSKNFHLRPRDLISNINVRVLELKGSETTFMCEYADETATIVEFLNRWITFCQSIISTLT |
| 328 | hIL-2 | APTSSSTKKTQLQLEHLLLDLQMILNGINNYKNPKLTEMLTFKFYMPKKATELKHLQCLEEELKPLEEV |
| | R38E/192N | LNLAQSKNFHLRPRDLISNINVNVLELKGSETTFMCEYADETATIVEFLNRWITFCQSIISTLT |
| 329 | hIL-2 | APTSSSTKKTQLQLEHLLLDLQMILNGINNYKNPKLTEMLTFKFYMPKKATELKHLQCLEEELKPLEEV |
| | R38E/192Q | LNLAQSKNFHLRPRDLISNINVQVLELKGSETTEMCEYADETATIVEFLNRWITFCQSIISTLT |
| 330 | hIL-2 | APTSSSTKKTQLQLEHLLLDLQMILNGINNYKNPKLTEMLTFKFYMPKKATELKHLQCLEEELKPLEEV |
| | R38E/I92E | LNLAQSKNFHLRPRDLISNINVEVLELKGSETTFMCEYADETATIVEFLNRWITFCQSIISTLT |
| 331 | hIL-2 | APTSSSTKKTQLQLEHLLLDLQMILNGINNYKNPKLTEMLTFKFYMPKKATELKHLQCLEEELKPLEEV |
| | R38E/I92G | LNLAQSKNFHLRPRDLISNINVGVLELKGSETTFMCEYADETATIVEFLNRWITFCQSIISTLT |
| 332 | hIL-2 | APTSSSTKKTQLQLEHLLLDLQMILNGINNYKNPKLTEMLTFKFYMPKKATELKHLQCLEEELKPLEEV |
| | R38E/192H | LNLAQSKNFHLRPRDLISNINVVLELKGSETTEMCEYADETATIVEFLNRWITFCQSIISTLT |
| 333 | hIL-2 | APTSSSTKKTQLQLEHLLLDLQMILNGINNYKNPKLTEMLTFKFYMPKKATELKHLQCLEEELKPLEEV |
| | R38E/192L | LNLAQSKNFHLRPRDLISNINVLVLELKGSETTFMCEYADETATIVEFLNRWITFCQSIISTLT |
| 334 | hIL-2 | APTSSSTKKTQLQLEHLLLDLQMILNGINNYKNPKLTEMLTFKFYMPKKATELKHLQCLEEELKPLEEV |
| | R38E/I92K | LNLAQSKNFHLRPRDLISNINVKVLELKGSETTFMCEYADETATIVEFLNRWITFCQSIISTLT |
| 335 | hIL-2 | APTSSSTKKTQLQLEHLLLDLQMILNGINNYKNPKLTEMLTFKFYMPKKATELKHLQCLEEELKPLEEV |
| | R38E/192M | LNLAQSKNFHLRPRDLISNINVMVLELKGSETTFMCEYADETATIVEFLNRWITFCQSIISTLT |
| 336 | hIL-2 | APTSSSTKKTQLQLEHLLLDLQMILNGINNYKNPKLTEMLTFKFYMPKKATELKHLQCLEEELKPLEEV |
| | R38E/I92F | LNLAQSKNFHLRPRDLISNINVFVLELKGSETTFMCEYADETATIVEFLNRWITFCQSIISTLT |
| 337 | hIL-2 | APTSSSTKKTQLQLEHLLLDLQMILNGINNYKNPKLTEMLTFKFYMPKKATELKHLQCLEEELKPLEEV |
| | R38E/192P | LNLAQSKNFHLRPRDLISNINVPVLELKGSETTEMCEYADETATIVEFLNRWITFCQSIISTLT |
| 338 | hIL-2 | APTSSSTKKTQLQLEHLLLDLQMILNGINNYKNPKLTEMLTFKFYMPKKATELKHLQCLEEELKPLEEV |
| | R38E/192S | LNLAQSKNFHLRPRDLISNINVSVLELKGSETTFMCEYADETATIVEFLNRWITFCQSIISTLT |
| 339 | hIL-2 | APTSSSTKKTQLQLEHLLLDLQMILNGINNYKNPKLTEMLTFKFYMPKKATELKHLQCLEEELKPLEEV |
| | R38E/192T | LNLAQSKNFHLRPRDLISNINVTVLELKGSETTEMCEYADETATIVEFLNRWITFCQSIISTLT |
| 340 | hIL-2 | APTSSSTKKTQLQLEHLLLDLQMILNGINNYKNPKLTEMLTFKFYMPKKATELKHLQCLEEELKPLEEV |
| | R38E/192W | LNLAQSKNFHLRPRDLISNINVWVLELKGSETTFMCEYADETATIVEFLNRWITFCQSIISTLT |
| 341 | hIL-2 | APTSSSTKKTQLQLEHLLLDLQMILNGINNYKNPKLTEMLTFKFYMPKKATELKHLQCLEEELKPLEEV |
| | R38E/I92Y | LNLAQSKNFHLRPRDLISNINVYVLELKGSETTFMCEYADETATIVEFLNRWITFCQSIISTLT |
| 342 | hIL-2 | APTSSSTKKTQLQLEHLLLDLQMILNGINNYKNPKLTEMLTFKFYMPKKATELKHLQCLEEELKPLEEV |
| | R38E/192V | LNLAQSKNFHLRPRDLISNINVVVLELKGSETTFMCEYADETATIVEFLNRWITFCQSIISTLT |
| 343 | hIL-2 | APTSSSTKKTQLQLEELLLDLQMILNGINNYKNPKLTEMLTFKFYMPKKATELKHLQCLEEELKPLEEV |
| | R38E/H16E | LNLAQSKNFHLRPRDLISNINVIVLELKGSETTFMCEYADETATIVEFLNRWITFCQSIISTLT |
| 344 | hIL-2 | APTSSSTKKTQLQLEHLLLALQMILNGINNYKNPKLTKMLTFKFYMPKKATELKHLQCLEEELKPLEEV |
| | R38K/D20A | LNLAQSKNFHLRPRDLISNINVIVLELKGSETTFMCEYADETATIVEFLNRWITFCQSIISTLT |
| 345 | WT hIL-2 | APTSSSTKKTQLQLEHLLLDLQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCQSIISTLT |
| 346 | Human PD-1 | PGWFLDSPDRPWNPPTESPALLVVTEGDNATFTCSESNTSESFVLNWYRMSPSNQTDKLAAFPEDRSOP |
| | | GQDCRFRVTQLPNGRDFHMSVVRARRNDSGTYLCGAISLAPKAQIKESLRAELRVTERRAEVPTAHPSP |
| | | SPRPAGQFQTLVVGVVGGLLGSLVLLVWVLAVICSRAARGTIGARRTGQPLKEDPSAVPVESVDYGELD |
| | | FQWREKTPEPPVPCVPEQTEYATIVEPSGMGTSSPARRGSADGPRSAQPLRPEDGHCSWPL |
| 347 | Human PD-1 | CCAGGATGGTTCTTAGACTCCCCAGACAGGCCCTGGAACCCCCCCACCTTCTCCCCAGCCCTGCTCGTG |
| | | GTGACCGAAGGGGACAACGCCACCTTCACCTGCAGCTTCTCCAACACATCGGAGAGCTTCGTGCTAAAC |
| | | TGGTACCGCATGAGCCCCAGCAACCAGACGGACAAGCTGGCCGCCTTCCCCGAGGACCGCAGCCAGCCC |
| | | GGCCAGGACTGCCGCTTCCGTGTCACACAACTGCCCAACGGGCGTGACTTCCACATGAGCGTGGTCAGG |
| | | GCCCGGCGCAATGACAGCGGCACCTACCTCTGTGGGGCCATCTCCCTGGCCCCCAAGGCGCAGATCAAA |
| | | GAGAGCCTGCGGGCAGAGCTCAGGGTGACAGAGAGAAGGGCAGAAGTGCCCACAGCCCACCCCAGCCCC |
| | | TCACCCAGGCCAGCCGGCCAGTTCCAAACCCTGGTGGTTGGTGTCGTGGGCGGCCTGCTGGGCAGCCTG |
| | | GTGCTGCTAGTCTGGGTCCTGGCCGTCATCTGCTCCCGGGCCGCACGAGGGACAATAGGAGCCAGGCGC |
| | | ACCGGCCAGCCCCTGAAGGAGGACCCCTCAGCCGTGCCTGTGTTCTCTGTGGACTATGGGGAGCTGGAT |
| | | TTCCAGTGGCGAGAGAAGACCCCGGAGCCCCCCGTGCCCTGTGTCCCTGAGCAGACGGAGTATGCCACC |
| | | ATTGTCTTTCCTAGCGGAATGGGCACCTCATCCCCCGCCCGCAGGGGCTCAGCTGACGGCCCTCGGAGT |
| | | GCCCAGCCACTGAGGCCTGAGGATGGACACTGCTCTTGGCCCCTC |
| 348 | Anti-hPD-1 #1- | QVQLVESGGGVVQPGRSLRLDCKASGITFSNSGMHWVRQAPGKGLEWVAVIWYDGSKRYYADSVKGRFT |
| | mIgG2b-N297A | ISRDNSKNTLFLQMNSLRAEDTAVYYCATNDDYWGQGTLVTVSSAKTTPPSVYPLAPGCGDTTGSSVTL |
| | HC | GCLVKGYFPESVTVTWNSGSLSSSVHTFPALLQSGLYTMSSSVTVPSSTWPSQTVTCSVAHPASSTTVD |
| | | KKLEPSGPISTINPCPPCKECHKCPAPNLEGGPSVFIFPPNIKDVLMISLTPKVTCVVVDVSEDDPDVQ |
| | | ISWFVNNVEVHTAQTQTHREDYASTIRVVSTLPIQHQDWMSGKEFKCKVNNKDLPSPIERTISKIKGLV |
| | | RAPQVYILPPPAEQLSRKDVSLTCLVVGENPGDISVEWTSNGHTEENYKDTAPVLDSDGSYFIYSKLNM |
| | | KTSKWEKTDSFSCNVRHEGLKNYYLKKTISRSPGK |
| 349 | Anti-hPD-1 #1- | EIVLTQSPATLSLSPGERATLSCRASQSVSSYLAWYQQKPGQAPRLLIYDASNRATGIPARFSGSGSGT |
| | mKappa LC | DFTLTISSLEPEDFAVYYCQQSSNWPRTFGQGTKVEIKRADAAPTVSIFPPSSEQLTSGGASVVCELNN |
| | | FYPKDINVKWKIDGSERQNGVLNSWTDQDSKDSTYSMSSTLTLTKDEYERHNSYTCEATHKTSTSPIVK |
| | | SFNRNEC |
| 350 | Anti-hPD-1 #2- | QVQLVQSGVEVKKPGASVKVSCKASGYTFTNYYMYWVRQAPGQGLEWMGGINPSNGGTNENEKFKNRVT |
| | mIgG2b-N297A | LTTDSSTTTAYMELKSLQFDDTAVYYCARRDYRFDMGEDYWGQGTTVTVSSAKTTPPSVYPLAPGCGDT |
| | HC | TGSSVTLGCLVKGYFPESVTVTWNSGSLSSSVHTFPALLQSGLYTMSSSVTVPSSTWPSQTVTCSVAHP |
| | | ASSTTVDKKLEPSGPISTINPCPPCKECHKCPAPNLEGGPSVFIFPPNIKDVLMISLTPKVTCVVVDVS |
| | | EDDPDVQISWFVNNVEVHTAQTQTHREDYASTIRVVSTLPIQHQDWMSGKEFKCKVNNKDLPSPIERTI |
| | | SKIKGLVRAPQVYILPPPAEQLSRKDVSLTCLVVGENPGDISVEWTSNGHTEENYKDTAPVLDSDGSYF |
| | | IYSKLNMKTSKWEKTDSFSCNVRHEGLKNYYLKKTISRSPGK |
| 351 | Anti-hPD-1 #2- | EIVLTQSPATLSLSPGERATLSCRASKGVSTSGYSYLHWYQQKPGQAPRLLIYLASYLESGVPARESGS |
| | mKappa LC | GSGTDFTLTISSLEPEDFAVYYCQHSRDLPLTFGGGTKVEIKRADAAPTVSIFPPSSEQLTSGGASVVC |
| | | FLNNFYPKDINVKWKIDGSERQNGVLNSWTDQDSKDSTYSMSSTLTLTKDEYERHNSYTCEATHKTSTS |
| | | PIVKSENRNEC |
| 352 | IL-2RY | LNTTILTPNGNEDTTADFFLTTMPTDSLSVSTLPLPEVQCEVENVEYMNCTWNSSSEPQPTNLTLHYWY |
| | | KNSDNDKVQKCSHYLFSEEITSGCQLQKKEIHLYQTFVVQLQDPREPRRQATQMLKLQNLVIPWAPENL |
| | | TLHKLSESQLELNWNNRFLNHCLEHLVQYRTDWDHSWTEQSVDYRHKFSLPSVDGQKRYTERVRSRENP |
| | | LCGSAQHWSEWSHPIHWGSNTSKENPFLFALEAVVISVGSMGLIISLLCVYFWLERTMPRIPTLKNLED |
| | | LVTEYHGNFSAWSGVSKGLAESLQPDYSERLCLVSEIPPKGGALGEGPGASPCNQHSPYWAPPCYTLKP |
| | | ET |
| 353 | human CD122 | AVNGTSQFTCFYNSRANISCVWSQDGALQDTSCQVHAWPDRRRWNQTCELLPVSQASWACNLILGAPDS |
| | (IL-2R) | QKLTTVDIVTLRVLCREGVRWRVMAIQDEKPFENLRLMAPISLQVVHVETHRCNISWEISQASHYFERH |
| | | LEFEARTLSPGHTWEEAPLLTLKQKQEWICLETLTPDTQYEFQVRVKPLQGEFTTWSPWSQPLAFRTKP |
| | | AALGKDTIPWLGHLLVGLSGAFGFIILVYLLINCRNTGPWLKKVLKCNTPDPSKFFSQLSSEHGGDVQK |
| | | WLSSPFPSSSESPGGLAPEISPLEVLERDKVTQLLLQQDKVPEPASLSSNHSLTSCFTNQGYFFFHLPD |
| | | ALEIEACQVYFTYDPYSEEDPDEGVAGAPTGSSPQPLQPLSGEDDAYCTFPSRDDLLLESPSLLGGPSP |
| | | PSTAPGGSGAGEERMPPSLQERVPRDWDPQPLGPPTPGVPDLVDFQPPPELVLREAGEEVPDAGPREGV |
| | | SFPWSRPPGQGEFRALNARLPLNTDAYLSLQELQGQDPTHLV |
| 354 | IL-2Rx | ELCDDDPPEIPHATFKAMAYKEGTMLNCECKRGFRRIKSGSLYMLCTGNSSHSSWDNQCQCTSSATRNT |
| | | TKQVTPQPEEQKERKTTEMQSPMQPVDQASLPGHCREPPPWENEATERIYHFVVGQMVYYQCVQGYRAL |
| | | HRGPAESVCKMTHGKTRWTQPQLICTGEMETSQFPGEEKPQASPEGRPESETSCLVTTTDFQIQTEMAA |
| | | TMETSIFTTEYQVAVAGCVFLLISVLLLSGLTWQRRQRKSRRTI |
| 355 | L6 linker | SGGGGS |
| | amino acid | |
| 356 | hIL-2-df-1H3- | APTSSSTKKTQLQLEHLLLDLQMILNGINNYKNPKLTRMLTFKFYMPKKATELKHLQCLEEELKPLEEV |
| | hkappa LC | LNLAQSKNFHLRPRDLISNINVIVLELKGSETTEMCEYADETATIVEFLNRWITFCOSIISTLTDTVLT |
| | hIL-2 in | QSPALAVSPGERVTISCRASESVRTGVHWYQQKPGQQPKLLIYGASNLESGVPARFSGSGSGTDETLTI |
| | italics | DPVEADDTATYFCQQSWNDPFTFGSGTKLEIKRTVAAPSVFIFPPSDEQLKSGTASVVCLLNNFYPREA |
| | | KVQWKVDNALQSGNSQESVTEQDSKDSTYSLSSTLTLSKADYEKHKVYACEVTHQGLSSPVTKSENRGE |
| | | C |
| 357 | hIL-2-L6-1H3- | APTSSSTKKTQLQLEHLLLDLOMILNGINNYKNPKLTRMLTEKFYMPKKATELKHLOCLEEELKPLEEV |
| | hKappa LC | LNLAQSKNFHLRPRDLISNINVIVLELKGSETTFMCEYADETATIVEFLNRWITFCQSIISTLT 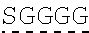 |
| | Linker in | SDTVLTQSPALAVSPGERVTISCRASESVRTGVHWYQQKPGQQPKLLIYGASNLESGVPARESGSGSGT |
| | dashed | DFTLTIDPVEADDTATYFCQQSWNDPFTFGSGTKLEIKRTVAAPSVFIFPPSDEQLKSGTASVVCLLNN |
| | underline | FYPREAKVQWKVDNALQSGNSQESVTEQDSKDSTYSLSSTLTLSKADYEKHKVYACEVTHQGLSSPVTK |
| | hIL-2 in | SENRGEC |
| | italics | |
| 358 | hIL-2-df-1H3- | APTSSSTKKTQLQLEHLLLDLQMILNGINNYKNPKLTRMLTFKFYMPKKATELKHLQCLEEELKPLEEV |
| | hIgG1 HC | LNLAQSKNFHLRPRDLISNINVIVLELKGSETTEMCEYADETATIVEFLNRWITFCQSIISTLTEVQLV |
| | hIL-2 in | ESGGGLVQPGRSLKLSCAVSGFTFSDYAMAWVRQAPKKGLEWVATISYDGSRTYYRDSVKGRFTISRDN |
| | italics | AKITLYLQMDSLRSEDTATYYCARHGSGYFDYWGQGVMVTVSSASTKGPSVFPLAPSSKSTSGGTAALG |
| | | CLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPEVTCVVVDVSHEDPEVKENWYVDG |
| | | VEVHNAKTKPREEQYNSTYRVVSVLTVLHQDWLNGKEYKCKVSNKALPAPIEKTISKAKGQPREPQVYT |
| | | LPPSRDELTKNQVSLTCLVKGFYPSDIAVEWESNGQPENNYKTTPPVLDSDGSFFLYSKLTVDKSRWQQ |
| | | GNVFSCSVMHEALHNHYTQKSLSLSPGK |
| 359 | hIL-2-L6-1H3- | APTSSSTKKTQLQLEHLLLDLQMILNGINNYKNPKLTRMLTFKFYMPKKATELKHLQCLEEELKPLEEV |
| | hIgG1 HC | LNLAQSKNFHLRPRDLISNINVIVLELKGSETTEMCEYADETATIVEFLNRWITFCQSIISTLT 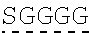 |
| | Linker in | SEVQLVESGGGLVQPGRSLKLSCAVSGFTFSDYAMAWVRQAPKKGLEWVATISYDGSRTYYRDSVKGRF |
| | dashed | TISRDNAKITLYLQMDSLRSEDTATYYCARHGSGYFDYWGQGVMVTVSSASTKGPSVFPLAPSSKSTSG |
| | underline | GTAALGCLVKDYFPEPVTVSWNSGALTSGVHTFPAVLQSSGLYSLSSVVTVPSSSLGTQTYICNVNHKP |
| | hIL-2 in | SNTKVDKKVEPKSCDKTHTCPPCPAPELLGGPSVFLFPPKPKDTLMISRTPEVTCVVVDVSHEDPEVKE |
| | italics | NWYVDGVEVHNAKTKPREEQYNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTTPPVLDSDGSFFLYSKLTVD |
| | | KSRWQQGNVFSCSVMHEALHNHYTQKSLSLSPGK |
| 360 | 1H3-hIgG1-df - | EVQLVESGGGLVQPGRSLKLSCAVSGFTFSDYAMAWVRQAPKKGLEWVATISYDGSRTYYRDSVKGRET |
| | hIL-2 HC | ISRDNAKITLYLOMDSLRSEDTATYYCARHGSGYFDYWGQGVMVTVSSASTKGPSVFPLAPSSKSTSGG |
| | hIL-2 in | TAALGCLVKDYFPEPVTVSWNSGALTSGVHTFPAVLQSSGLYSLSSVVTVPSSSLGTQTYICNVNHKPS |
| | italics | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKAPTSSSTKKTQLQLEHLLLDLQMILNGINNYKNPKL |
| | | TRMLTFKFYMPKKATELKHLQCLEEELKPLEEVLNLAQSKNFHLRPRDLISNINVIVLELKGSETTEMC |
| | | EYADETATIVEFLNRWITFCQSIISTLT |
| 361 | 1H3-hIgG1-L6- | EVQLVESGGGLVQPGRSLKLSCAVSGFTESDYAMAWVRQAPKKGLEWVATISYDGSRTYYRDSVKGRET |
| | hIL-2 HC | ISRDNAKITLYLQMDSLRSEDTATYYCARHGSGYFDYWGQGVMVTVSSASTKGPSVFPLAPSSKSTSGG |
| | Linker in | TAALGCLVKDYFPEPVTVSWNSGALTSGVHTFPAVLQSSGLYSLSSVVTVPSSSLGTQTYICNVNHKPS |
| | dashed | NTKVDKKVEPKSCDKTHTCPPCPAPELLGGPSVFLFPPKPKDTLMISRTPEVTCVVVDVSHEDPEVKEN |
| | underline | WYVDGVEVHNAKTKPREEQYNSTYRVVSVLTVLHQDWLNGKEYKCKVSNKALPAPIEKTISKAKGQPRE |
| | hIL-2 in | PQVYTLPPSRDELTKNQVSLTCLVKGFYPSDIAVEWESNGQPENNYKTTPPVLDSDGSFFLYSKLTVDK |
| | italics | SRWQQGNVFSCSVMHEALHNHYTQKSLSLSPGK 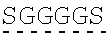 APTSSSTKKTQLQLEHLLLDLQMILNGI |
| | | NNYKNPKLTRMLTFKFYMPKKATELKHLQCLEEELKPLEEVLNLAQSKNFHLRPRDLISNINVIVLEL |
| | | KGSETTEMCEYADETATIVEFLNRWITFCQSIISTLT |
| 362 | 1H3-hKappa-df- | DTVLTQSPALAVSPGERVTISCRASESVRTGVHWYQQKPGQQPKLLIYGASNLESGVPARFSGSGSGTD |
| | hIL-2 (WT) LC | FTLTIDPVEADDTATYFCQQSWNDPFTFGSGTKLEIKRTVAAPSVFIFPPSDEQLKSGTASVVCLLNNE |
| | hIL-2 in | YPREAKVQWKVDNALQSGNSQESVTEQDSKDSTYSLSSTLTLSKADYEKHKVYACEVTHQGLSSPVTKS |
| | italics | FNRGECAPTSSSTKKTQLQLEHLLLDLQMILNGINNYKNPKLTRMLTEKFYMPKKATELKHLQCLEEEL |
| | | KPLEEVLNLAQSKNFHLRPRDLISNINVIVLELKGSETTFMCEYADETATIVEFLNRWITFCQSIISTL |
| | | T |
| 363 | 1H3-hKappa-L6- | DTVLTQSPALAVSPGERVTISCRASESVRTGVHWYQQKPGQQPKLLIYGASNLESGVPARFSGSGSGTD |
| | hIL-2 (WT) LC | FTLTIDPVEADDTATYFCQQSWNDPFTFGSGTKLEIKRTVAAPSVFIFPPSDEQLKSGTASVVCLLNNE |
| | Linker in | YPREAKVQWKVDNALQSGNSQESVTEQDSKDSTYSLSSTLTLSKADYEKHKVYACEVTHQGLSSPVTKS |
| | dashed | FNRGEC 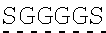 APTSSSTKKTQLQLEHLLLDLQMILNGINNYKNPKLTRMLTEKFYMPKKATELKH |
| | underline | LQCLEEELKPLEEVLNLAQSKNFHLRPRDLISNINVIVLELKGSETTEMCEYADETATIVEFLNRWITF |
| | | CQSIISTLT |
| | hIL-2 in | |
| | italics | |
| 364 | L20 linker | SGGGGSGGGGSGGGGSGGGS |
| 365 | hCD25-L20-hIL- | ELCDDDPPEIPHATFKAMAYKEGTMLNCECKRGERRIKSGSLYMLCTGNSSHSSWDNQCQCTSSATRNT |
| | 2-df-1H3-hIgG1 | TKQVTPQPEEQKERKTTEMQSPMQPVDQASLPGHCREPPPWENEATERIYHFVVGQMVYYQCVQGYRAL |
| | HC | HRGPAESVCKMTHGKTRWTQPQLICT 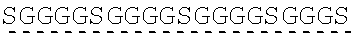 APTSSSTKKTQLQLEHLLLDL |
| | Linker in | QMILNGINNYKNPKLTRMLTFKFYMPKKATELKHLQCLEEELKPLEEVLNLAQSKNFHLRPRDLISNIN |
| | dashed | VIVLELKGSETTFMCEYADETATIVEFLNRWITFCQSIISTLTEVQLVESGGGLVQPGRSLKLSCAVSG |
| | underline | ETFSDYAMAWVRQAPKKGLEWVATISYDGSRTYYRDSVKGRFTISRDNAKITLYLQMDSLRSEDTATYY |
| | hIL-2 in | CARHGSGYFDYWGQGVMVTVSSASTKGPSVFPLAPSSKSTSGGTAALGCLVKDYFPEPVTVSWNSGALT |
| | italics | SGVHTFPAVLQSSGLYSLSSVVTVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKENWYVDGVEVHNAKTKPREEQYNSTYRV |
| | | VSVLTVLHQDWLNGKEYKCKVSNKALPAPIEKTISKAKGQPREPQVYTLPPSRDELTKNQVSLTCLVKG |
| | | FYPSDIAVEWESNGQPENNYKTTPPVLDSDGSFFLYSKLTVDKSRWQQGNVFSCSVMHEALHNHYTQKS |
| | | LSLSPGK |
| 366 | hCD25-L20-hIL- | ELCDDDPPEIPHATFKAMAYKEGTMLNCECKRGFRRIKSGSLYMLCTGNSSHSSWDNQCQCTSSATRNT |
| | 2-L6-1H3-hIgG1 | TKQVTPQPEEQKERKTTEMQSPMQPVDQASLPGHCREPPPWENEATERIYHFVVGQMVYYQCVQGYRAL |
| | HC | HRGPAESVCKMTHGKTRWTQPQLICT 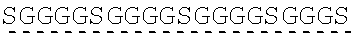 APTSSSTKKTQLQLEHLLLDL |
| | Linkers in | QMILNGINNYKNPKLTRMLTFKFYMPKKATELKHLQCLEEELKPLEEVLNLAQSKNFHLRPRDLISNIN |
| | dashed | VIVLELKGSETTEMCEYADETATIVEFLNRWITFCQSIISTLTSGGGGSEVOLVESGGGLVQPGRSLKL |
| | underline | SCAVSGFTFSDYAMAWVRQAPKKGLEWVATISYDGSRTYYRDSVKGRFTISRDNAKITLYLQMDSLRSE |
| | hIL-2 in | DTATYYCARHGSGYFDYWGQGVMVTVSSASTKGPSVFPLAPSSKSTSGGTAALGCLVKDYFPEPVTVSW |
| | italics | NSGALTSGVHTFPAVLQSSGLYSLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKENWYVDGVEVHNAKTKPREEQY |
| | | NSTYRVVSVLTVLHQDWLNGKEYKCKVSNKALPAPIEKTISKAKGQPREPQVYTLPPSRDELTKNQVSL |
| | | TCLVKGFYPSDIAVEWESNGQPENNYKTTPPVLDSDGSFFLYSKLTVDKSRWQQGNVFSCSVMHEALHN |
| | | HYTQKSLSLSPGK |
| 367 | hCD25-L20-hIL- | ELCDDDPPEIPHATFKAMAYKEGTMLNCECKRGFRRIKSGSLYMLCTGNSSHSSWDNQCQCTSSATRNT |
| | 2-df-1H3- | TKQVTPQPEEQKERKTTEMQSPMQPVDQASLPGHCREPPPWENEATERIYHFVVGQMVYYQCVQGYRAL |
| | hKappa LC | HRGPAESVCKMTHGKTRWTQPQLICT 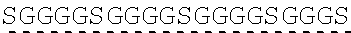 APTSSSTKKTQLQLEHLLLDL |
| | Linker in | QMILNGINNYKNPKLTRMLTFKFYMPKKATELKHLQCLEEELKPLEEVLNLAQSKNFHLRPRDLISNIN |
| | dashed | VIVLELKGSETTEMCEYADETATIVEFLNRWITFCQSIISTLTDTVLTQSPALAVSPGERVTISCRASE |
| | underline | SVRTGVHWYQQKPGQQPKLLIYGASNLESGVPARFSGSGSGTDFTLTIDPVEADDTATYFCQQSWNDPF |
| | hIL-2 in | TFGSGTKLEIKRTVAAPSVFIFPPSDEQLKSGTASVVCLLNNFYPREAKVQWKVDNALQSGNSQESVTE |
| | italics | QDSKDSTYSLSSTLTLSKADYEKHKVYACEVTHQGLSSPVTKSENRGEC |
| 368 | hCD25-L20-hIL- | ELCDDDPPEIPHATFKAMAYKEGTMLNCECKRGERRIKSGSLYMLCTGNSSHSSWDNQCQCTSSATRNT |
| | 2-L6-1H3- | TKQVTPQPEEQKERKTTEMQSPMQPVDQASLPGHCREPPPWENEATERIYHFVVGQMVYYQCVQGYRAL |
| | hKappa LC | HRGPAESVCKMTHGKTRWTQPQLICT 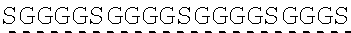 APTSSSTKKTQLQLEHLLLDL |
| | Linkers in | QMILNGINNYKNPKLTRMLTFKFYMPKKATELKHLQCLEEELKPLEEVLNLAQSKNFHLRPRDLISNI |
| | dashed | NVIVLELKGSETTFMCEYADETATIVEFLNRWITFCQSIISTLT 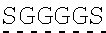 DTVLTQSPALAVSPGER |
| | underline | VTISCRASESVRTGVHWYQQKPGQQPKLLIYGASNLESGVPARFSGSGSGTDFTLTIDPVEADDTATYF |
| | hIL-2 in | CQQSWNDPFTFGSGTKLEIKRTVAAPSVFIFPPSDEQLKSGTASVVCLLNNFYPREAKVQWKVDNALQS |
| | italics | GNSQESVTEQDSKDSTYSLSSTLTLSKADYEKHKVYACEVTHQGLSSPVTKSENRGEC |
| 369 | 1H3-hIgG1-df- | EVOLVESGGGLVQPGRSLKLSCAVSGFTFSDYAMAWVRQAPKKGLEWVATISYDGSRTYYRDSVKGRET |
| | hCD25-L20-hIL- | ISRDNAKITLYLQMDSLRSEDTATYYCARHGSGYFDYWGQGVMVTVSSASTKGPSVFPLAPSSKSTSGG |
| | 2 HC | TAALGCLVKDYFPEPVTVSWNSGALTSGVHTFPAVLQSSGLYSLSSVVTVPSSSLGTQTYICNVNHKPS |
| | Linker in | NTKVDKKVEPKSCDKTHTCPPCPAPELLGGPSVFLFPPKPKDTLMISRTPEVTCVVVDVSHEDPEVKEN |
| | dashed | WYVDGVEVHNAKTKPREEQYNSTYRVVSVLTVLHQDWLNGKEYKCKVSNKALPAPIEKTISKAKGQPRE |
| | underline | PQVYTLPPSRDELTKNQVSLTCLVKGFYPSDIAVEWESNGQPENNYKTTPPVLDSDGSFFLYSKLTVDK |
| | hIL-2 in | SRWQQGNVFSCSVMHEALHNHYTQKSLSLSPGKELCDDDPPEIPHATFKAMAYKEGTMLNCECKRGERR |
| | italics | IKSGSLYMLCTGNSSHSSWDNQCQCTSSATRNTTKQVTPQPEEQKERKTTEMQSPMQPVDQASLPGHCR |
| | | EPPPWENEATERIYHFVVGQMVYYQCVQGYRALHRGPAESVCKMTHGKTRWTQPQLICT 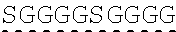 |
| | | 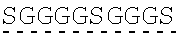 APTSSSTKKTQLQLEHLLLDLOMILNGINNYKNPKLTRMLTFKFYMPKKATEL |
| | | KHLQCLEEELKPLEEVLNLAQSKNFHLRPRDLISNINVIVLELKGSETTEMCEYADETATIVEFLNRW |
| | | ITFCQSIISTLT |
| 370 | 1H3-hIgG1-L6- | EVQLVESGGGLVQPGRSLKLSCAVSGFTFSDYAMAWVRQAPKKGLEWVATISYDGSRTYYRDSVKGRET |
| | hCD25-L20-hIL- | ISRDNAKITLYLQMDSLRSEDTATYYCARHGSGYFDYWGQGVMVTVSSASTKGPSVFPLAPSSKSTSGG |
| | 2 HC | TAALGCLVKDYFPEPVTVSWNSGALTSGVHTFPAVLQSSGLYSLSSVVTVPSSSLGTQTYICNVNHKPS |
| | Linkers in | NTKVDKKVEPKSCDKTHTCPPCPAPELLGGPSVFLFPPKPKDTLMISRTPEVTCVVVDVSHEDPEVKEN |
| | dashed | WYVDGVEVHNAKTKPREEQYNSTYRVVSVLTVLHQDWLNGKEYKCKVSNKALPAPIEKTISKAKGQPRE |
| | underline | PQVYTLPPSRDELTKNQVSLTCLVKGFYPSDIAVEWESNGQPENNYKTTPPVLDSDGSFFLYSKLTVDK |
| | hIL-2 in | SRWQQGNVFSCSVMHEALHNHYTQKSLSLSPGK 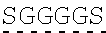 ELCDDDPPEIPHATFKAMAYKEGTMLNCE |
| | italics | CKRGFRRIKSGSLYMLCTGNSSHSSWDNQCQCTSSATRNTTKQVTPQPEEQKERKTTEMQSPMQPVDQA |
| | | S 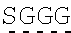 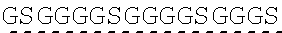 LPGHCREPPPWENEATERIYHFVVGQMVYYQCVQGYRALH |
| | | RGPAESVCKMTHGKTRWTQPQLICTAPTSSSTKKTQLQLEHLLLDLQMILNGINNY |
| | | KNPKLTRMLTFKFYMPKKATELKHLQCLEEELKPLEEVLNLAQSKNFHLRPRDLISNINVIVLELKGSE |
| | | TTEMCEYADETATIVEFLNRWITFCQSIISTLT |
| 371 | 1H3-hKappa-df- | DTVLTQSPALAVSPGERVTISCRASESVRTGVHWYQQKPGQQPKLLIYGASNLESGVPARFSGSGSGTD |
| | hCD25-L20-hIL- | FTLTIDPVEADDTATYFCQQSWNDPFTFGSGTKLEIKRTVAAPSVFIFPPSDEQLKSGTASVVCLLNNE |
| | 2 LC | YPREAKVQWKVDNALQSGNSQESVTEQDSKDSTYSLSSTLTLSKADYEKHKVYACEVTHQGLSSPVTKS |
| | Linker in | FNRGECELCDDDPPEIPHATFKAMAYKEGTMLNCECKRGERRIKSGSLYMLCTGNSSHSSWDNQCQCTS |
| | dashed | SATRNTTKQVTPQPEEQKERKTTEMQSPMQPVDQASLPGHCREPPPWENEATERIYHFVVGQMVYYQCV |
| | underline | QGYRALHRGPAESVCKMTHGKTRWTQPQLICT 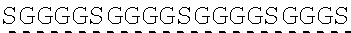 APTSSSTKKT |
| | hIL-2 in | QLQLEHLLLDLQMILNGINNYKNPKLTRMLTFKFYMPKKATELKHLQCLEEELKPLEEVLNL |
| | italics | AQSKNFHLRPRDLISNINVIVLELKGSETTEMCEYADETATIVEFLNRWITFCQSIISTLT |
| 372 | 1H3-hKappa-L6- | DTVLTQSPALAVSPGERVTISCRASESVRTGVHWYQQKPGQQPKLLIYGASNLESGVPARFSGSGSGTD |
| | hCD25-L20-hIL- | FTLTIDPVEADDTATYFCQQSWNDPFTFGSGTKLEIKRTVAAPSVFIFPPSDEQLKSGTASVVCLLNNE |
| | 2 LC | YPREAKVQWKVDNALQSGNSQESVTEQDSKDSTYSLSSTLTLSKADYEKHKVYACEVTHQGLSSPVTKS |
| | Linkers in | FNRGEC 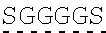 ELCDDDPPEIPHATFKAMAYKEGTMLNCECKRGFRRIKSG |
| | dashed | SLYMLCTGNSSHSSWD |
| | underline | NQCQCTSSATRNTTKQVTPQPEEQKERKTTEMQSPMQPVDQASLPGHCREPPPWENEATERIYHFVVGQ |
| | hIL-2 in | MVYYQCVQGYRALHRGPAESVCKMTHGKTRWTQPQLICT 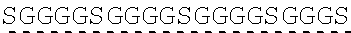 |
| | italics | APTSSSTKKTQLQLEHLLLDLQMILNGINNYKNPKLTRMLTFKFYMPKKAT |
| | | ELKHLQCLEEELKPLEEVLNLAQSKNFHLRPRDLISNINVIVLELKGSETTFMCEYAD |
| | | ETATIVEFLNRWITFCQSIISTLT |
| 373 | H7-02-hIgG4 HC | EVQLLESGGGLVQPGGSLRLSCAASGFTFKSYAMTWVRQAPGKGLEWVSAIVYSGGSTYYADSVKGRET |
| | | ISRDNSKNTLYLQMNSLRAEDTAVYYCAKYTRASYFYDAMDVWGQGTTVTVSSASTKGPSVFPLAPCSR |
| | | STSESTAALGCLVKDYFPEPVTVSWNSGALTSGVHTFPAVLQSSGLYSLSSVVTVPSSSLGTKTYTCNV |
| | | DHKPSNTKVDKRVESKYGPPCPPCPAPEFLGGPSVFLFPPKPKDTLMISRTPEVTCVVVDVSQEDPEVQ |
| | | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVESCSVMHEALHNHYTQKSLSLSLG |
| 374 | 1H3-hkappa LC | DTVLTQSPALAVSPGERVTISCRASESVRTGVHWYQQKPGQQPKLLIYGASNLESGVPARESGSGSGTD |
| | | FTLTIDPVEADDTATYFCQQSWNDPFTFGSGTKLEIKRTVAAPSVFIFPPSDEQLKSGTASVVCLLNNE |
| | | YPREAKVQWKVDNALQSGNSQESVTEQDSKDSTYSLSSTLTLSKADYEKHKVYACEVTHQGLSSPVTKS |
| | | ENRGEC |
| 375 | 2D12-mIgG1- | EVQLQQSGPELVKPGASVKISCKTSGYTFTEYTMHWVKQSHGKSLEWIGGINPNNGGTTYNQKEKGKAT |
| | D265A-L6-hIL-2 | LTVDKSSSTAYMELRSLTSQDSAVYYCARDYYRYGHYYAMDYWGQGTSVTVSSAKTTPPSVYPLAPGSA |
| | HC | AQTNSMVTLGCLVKGYFPEPVTVTWNSGSLSSGVHTFPAVLQSDLYTLSSSVTVPSSTWPSETVTCNVA |
| | | HPASSTKVDKKIVPRDCGCKPCICTVPEVSSVFIFPPKPKDVLTITLTPKVTCVVVAISKDDPEVQESW |
| | | FVDDVEVHTAQTQPREEQFNSTERSVSELPIMHQDWLNGKEFKCRVNSAAFPAPIEKTISKTKGRPKAP |
| | | QVYTIPPPKEQMAKDKVSLTCMITDFFPEDITVEWQWNGQPAENYKNTQPIMDTDGSYFVYSKLNVQKS |
| | | NWEAGNTFTCSVLHEGLHNHHTEKSLSHSPGKSGGGGSAPTSSSTKKTQLQLEHLLLDLQMILNGINNY |
| | | KNPKLTRMLTFKFYMPKKATELKHLOCLEEELKPLEEVLNLAQSKNFHLRPRDLISNINVIVLELKGSE |
| | | TTFMCEYADETATIVEFLNRWITFCQSIISTLT |
| 376 | 2D12-mkappa LC | QIVLTQSPAIMSASPGEKVTMTCSVSSSVREMHWYQQKSGTSPKRWIYDTSKLASGVPARESGSGSGTS |
| | | YSLTISSMEAEDAATYYCQQWSSNPPTFGGGTKLKIKRADAAPTVSIFPPSSEQLTSGGASVVCFLNNF |
| | | YPKDINVKWKIDGSERQNGVLNSWTDQDSKDSTYSMSSTLTLTKDEYERHNSYTCEATHKTSTSPIVKS |
| | | FNRNEC |
| 377 | hIL-2 Q126L | APTSSSTKKTQLQLEHLLLDLQMILNGINNYKNPKLTRMLTFKFYMPKKATELKHLQCLEEELKPLEEV |
| | | LNLAQSKNFHLRPRDLISNINVIVLELKGSETTEMCEYADETATIVEFLNRWITFCLSIISTLT |
| 378 | hIL-2 Q126E | APTSSSTKKTQLQLEHLLLDLQMILNGINNYKNPKLTRMLTFKFYMPKKATELKHLQCLEEELKPLEEV |
| | | LNLAQSKNFHLRPRDLISNINVIVLELKGSETTFMCEYADETATIVEFLNRWITFCESIISTLT |
| 379 | 1H3-hIgG1 HC | EVOLVESGGGLVQPGRSLKLSCAVSGFTFSDYAMAWVRQAPKKGLEWVATISYDGSRTYYRDSVKGRFT |
| | | ISRDNAKITLYLQMDSLRSEDTATYYCARHGSGYFDYWGQGVMVTVSSASTKGPSVFPLAPSSKSTSGG |
| | | TAALGCLVKDYFPEPVTVSWNSGALTSGVHTFPAVLQSSGLYSLSSVVTVPSSSLGTQTYICNVNHKPS |
| | | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGK |
| 380 | huPD-1-Fc | MQIPQAPWPVVWAVLQLGWRPGWELDSPDRPWNPPTFSPALLVVTEGDNATFTCSFSNTSESFVLNWYR |
| | | MSPSNQTDKLAAFPEDRSQPGQDCRFRVTQLPNGRDFHMSVVRARRNDSGTYLCGAISLAPKAQIKESL |
| | | RAELRVTERRAEVPTAHPSPSPRPAGQFQIEGRMDPKSCDKTHTCPPCPAPELLGGPSVELFPPKPKDT |
| | | LMISRTPEVTCVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYTQKSLSLSPGK |
| 381 | cynomolgous- | MQIPQAPWPVVWAVLQLGWRPGWFLESPDRPWNAPTESPALLLVTEGDNATFTCSFSNASESFVLNWYR |
| | PD-1-Fc | MSPSNQTDKLAAFPEDRSQPGQDCRFRVTRLPNGRDFHMSVVRARRNDSGTYLCGAISLAPKAQIKESL |
| | | RAELRVTERRAEVPTAHPSPSPRPAGQFQIEGRMDPKSCDKTHTCPPCPAPELLGGPSVFLFPPKPKDT |
| | | LMISRTPEVTCVVVDVSHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHYTQKSLSLSPGK |
| 382 | 2H7 VH | GAGGTGCAGCTGCTGGAAAGCGGCGGCGGACTGGTGCAGCCTGGAGGCAGCCTGCGGCTGTCTTGTGCC |
| | | GCTTCTGGCTTCACCTTCAAGGACTACTGCATGACCTGGGTCAGACAGGCCCCTGGCAAGGGCCTCGAG |
| | | TGGGTGTCCGCCATCGTGTACAGCGGCGGGTCAACATACTACGCCGACAGCGTGAAGGGCAGATTCACA |
| | | ATCAGCAGAGATAACAGCAAGAACACCCTGTACCTGCAGATGAACAACCTGAGAGCTGAAGATACCGCC |
| | | GTGTACTACTGCGCCAAGTACACCAGAGCCAGCTACTTCTACGACGCCATGGACGTGTGGGGCCAGGGC |
| | | ACCACCGTGACAGTGTCCTCAT |
| 383 | 2H7 VL | GAGATCGTGCTGACCCAGTCTCCTGGCACCCTGAGCCTGAGCCCTGGCGAGAGAGCTACACTGTCATGC |
| | | AGAGCCTCTCAGAGCATCGGCAAGAGCTTCCTGGCCTGGTACCAGCAAAAGCCTGGACAGGCCCCTAGA |
| | | CTGCTGATCTACGACGCCAGCACCAGAGCCGCTGATATCCCCGCCAGATTCAGCGGATCTGGCAGCGGC |
| | | ACTGATTTCACCCTCACCATCAGCAGCCTGGAACCCGAGGACTTCGCCGTGTACTACTGCCAGCAGTAC |
| | | TACGACTGGCCCCCCCTGTCTTTTGGCGGAGGCACAAAGGTGGAAATCAAG |
| 384 | 2H7 VH | EVQLLESGGGLVQPGGSLRLSCAASGFTFKDYCMTWVRQAPGKGLEWVSAIVYSGGSTYYADSVKGRFT |
| | | ISRDNSKNTLYLQMNNLRAEDTAVYYCAKYTRASYFYDAMDVWGQGTTVTVSS |
| 385 | 2H7 VL | EIVLTQSPGTLSLSPGERATLSCRASQSIGKSFLAWYQQKPGQAPRLLIYDASTRAADIPARFSGSGSG |
| | | TDFTLTISSLEPEDFAVYYCQQYYDWPPLSFGGGTKVEIK |
| 386 | 2H7 HCDR1 | GFT FKDY CMT |
| 387 | 2H7 HCDR2 | AIVYSGGSTYYADSVKG |
| 388 | 2H7 HCDR3 | YTRASYFYDAMDV |
| 389 | 2H7 LCDR1 | RASQSIGKSFLA |
| 390 | 2H7 LCDR2 | DASTRAA |
| 391 | 2H7 LCDR3 | QQYYDWPPLS |
| 392 | C51E6-5 VH | CAGGTTCAGCTGGTTCAGTCTGGCAGCGAGCTGAAGAAACCTGGCGCCTCTGTGAAGGTGTCCTGCAAG |
| | | GCCTCTGGCTACAGCCTGTACGGCACCTCTATGCACTGGGTCCGACAGGCTCCAGGACAGGGACTTGAG |
| | | TGGATGGGCTACATCAGCCCCTTTACCGGCAGAGCCACATACGCCCAGGGCTTCACAGGCAGATTCGTG |
| | | TTCAGCCTGGACACCAGCGTGTCCACAGCCTACCTGCAGATCAGCTCTCTGAAGGCCGAGGACACCGCC |
| | | GTGTACTACTGCGCCAGAGACTACGACTACCGGTACTACTATGCCATGGACTACTGGGGCCAGGGCACC |
| | | ACAGTTACAGTGTCCTCA |
| 393 | C51E6-5 VL | GAAATTGTGCTGACACAGAGCCCCGACTTCCAGAGCGTGACCCCTAAAGAAAAAGTGACCATCACCTGT |
| | | ACCGCCAGCGAGTCCGTGCCTCCTCAGTTCCTGCATTGGTATCAGCAGAAGCCCGATCAGAGCCCCAAG |
| | | CTGCTGATCTACGCCAGCAGAGAAAGAGCCAGCGGCGTCCCAAGCAGATTTTCTGGCTCTGGCAGCGGC |
| | | ACCGACTTCACCCTGACAATCAATAGCCTGGAAGCCGAGGACGCCGCCACCTACTACTGCCACCAGTTT |
| | | CACAGAAGCCCTCTGACCTTTGGCGGAGGCACCAAGCTGGAAATCAAG |
| 394 | C51E6-5 VH | QVQLVQSGSELKKPGASVKVSCKASGYSLYGTSMHWVRQAPGQGLEWMGYISPFTGRATYAQGFTGREV |
| | | FSLDTSVSTAYLQISSLKAEDTAVYYCARDYDYRYYYAMDYWGQGTTVTVSS |
| 395 | C51E6-5 VL | EIVLTQSPDFQSVTPKEKVTITCTASESVPPQFLHWYQQKPDQSPKLLIYASRERASGVPSRESGSGSG |
| | | TDFTLTINSLEAEDAATYYCHQFHRSPLTFGGGTKLEIK |
| 396 | C51E6-5 HCDR1 | GYSLYGTSMH |
| 397 | C51E6-5 HCDR2 | YISPFTGRATYAQGETG |
| 398 | C51E6-5 HCDR3 | DYDYRYYYAMDY |
| 399 | C51E6-5 LCDR1 | TASESVPPQFLH |
| 400 | C51E6-5 LCDR2 | ASRERAS |
| 401 | C51E6-5 LCDR3 | HOFHRSPLT |
| 402 | A2 VH | GACGTGCAGCTGGTGGAAAGCGGCGGAGGCCTGGTCCAGCCCGGCGGCTCTCTGAGACTGAGCTGCGCC |
| | | GCCAGCGGCTTCACCTTCGACATCAGCGCCATGAGCTGGGTGCGGCAGGCCCCTGGCAAGGGCCTGGAA |
| | | TGGGTCAGCACAATCAGCGGATCTGCCTACAGCACCTACTACGCCGACAGCGTGAAGGGCAGATTCACC |
| | | ATCTCAAGAGATAACAGCAAGAGCACCCTGTACCTGCAGATGAACAGCCTGCGGGCCGAGGACACCGCC |
| | | GTGTACTACTGCGCCAGAGAGATCTTCAGCGACTACTGGGGCTTGGGCACCCTGGTGACAGTGTCCTCA |
| 403 | A2 VL | CAAAGCGTGCTGACACAGCCCCCCAGCGCTTCTGGCACCCCTGGCCAGAGAGTGACCATCTCATGCAGC |
| | | GGGTCAACAAGCAACATCGGCAGAGAGAGCGTGTACTGGTACCAGCAGCTGCCTGGAACCGCCCCTAAG |
| | | CTGCTGATCTACAGCAACGTGCAGCGGCCTAGCGGCGCCCCTAACAGATTCAGCGGCAGCAAGAGCGGC |
| | | ACCAGCGCCAGCCTGGCCATCAGCGGCCTGCAGAGCGAGGACGAGGCCGACTACTACTGCGGCACATGG |
| | | GACGACAGCCTGAACGGCTGGGTGTTCGGCGGCGGAACTAAGCTGACCGTCCTA |
| 404 | A2 VH | DVQLVESGGGLVQPGGSLRLSCAASGFTFDISAMSWVRQAPGKGLEWVSTISGSAYSTYYADSVKGRFT |
| | | ISRDNSKSTLYLQMNSLRAEDTAVYYCAREIFSDYWGLGTLVTVSS |
| 405 | A2 VL | QSVLTQPPSASGTPGQRVTISCSGSTSNIGRESVYWYQQLPGTAPKLLIYSNVQRPSGAPNRESGSKSG |
| | (OMC479p1.A2VL) | TSASLAISGLQSEDEADYYCGTWDDSLNGWVFGGGTKLTVL |
| 406 | A2 HCDR1 | GFTFDISAMS |
| 407 | A2 HCDR2 | TISGSAYSTYYADSVKG |
| 408 | A2 HCDR3 | EIFSDY |
| 409 | A2 LCDR1 | SGSTSNIGRESVY |
| 410 | A2 LCDR2 | SNVQRPS |
| 411 | A2 LCDR3 | GTWDDSLNGWV |
| 412 | H7-767 HC | GAGGTGCAGCTGCTGGAAAGCGGCGGCGGCCTCGTGCAGCCTGGCGGATCTCTGCGGCTGAGCTGTGCT |
| | | GCCAGCGGCTTCACATTTAAATCCTACGCCATGCACTGGGTTAGACAAGCCCCCGGAAAGGGCCTGGAA |
| | | TGGGTGTCCGCCATCGTCTACAGCGGCGGATCTACATACTACGCCGACAGCGTGAAGGGCCGGTTCACC |
| | | ATCAGCAGAGATAATAGCAAGAACACCCTGTACCTGCAGATGAACAGCCTGAGAGCCGAGGACACCGCC |
| | | GTGTACTACTGCGCCAAGTACGACAGAGCTTCTTATTTCTACGATGCCATGGACGTGTGGGGCCAGGGC |
| | | ACCACCGTGACAGTGTCCTCAGCTAGCACCAAGGGCCCTAGCGTGTTTCCACTGGCCCCTAGCTCTAAA |
| | | AGCACAAGCGGCGGAACCGCCGCTCTGGGTTGTCTGGTGAAGGACTACTTCCCTGAGCCTGTGACCGTC |
| | | AGCTGGAACAGCGGCGCCCTGACCAGCGGCGTTCACACATTCCCCGCTGTGCTGCAGAGCTCTGGGCTG |
| | | TACAGCCTGAGCAGCGTGGTGACCGTGCCTTCTTCTTCTCTGGGCACACAAACATACATCTGCAACGTG |
| | | AACCACAAGCCCAGTAATACCAAAGTGGATAAGAAGGTGGAACCTAAGTCTTGCGACAAGACCCACACC |
| | | TGTCCTCCGTGCCCTGCTCCTGAACTGgctGGAgctCCCAGCGTGTTCCTGTTCCCCCCCAAACCTAAA |
| | | GACACCCTGATGATCAGCCGGACCCCTGAGGTGACCTGCGTGGTCGTCGACGTGTCCCACGAAGATCCT |
| | | GAGGTGAAGTTCAACTGGTACGTGGACGGCGTGGAAGTGCATAATGCCAAGACAAAGCCTAGAGAGGAA |
| | | CAGTACAACAGCACCTATAGAGTGGTGTCCGTGCTGACAGTGCTGCACCAGGACTGGCTGAACGGCAAG |
| | | GAATACAAGTGCAAGGTGTCCAACAAGGCCCTCCCCGCCCCTATCGAGAAGACCATCAGCAAGGCAAAG |
| | | GGCCAACCTAGAGAGCCCCAGGTGTACACCCTGCCTCCAAGCAGAGATGAGCTGACCAAGAACCAGGTT |
| | | AGCCTGACTTGTCTGGTGAAAGGCTTCTACCCCTCCGATATCGCCGTGGAATGGGAGAGCAACGGCCAG |
| | | CCTGAGAACAACTACAAGACCACACCTCCAGTGCTGGACAGCGACGGCAGCTTCTTCCTGTATAGCAAG |
| | | CTGACAGTGGACAAGAGCAGATGGCAGCAGGGCAACGTGTTCAGCTGCAGCGTCATGCACGAGGCCCTG |
| | | CACAACCACTACACCCAGAAGTCTCTGAGCCTGAGCCCTGGAAAGGCCCCTGCTTCTAGCAGCACCAAG |
| | | AAGACCCAGCTGCAGCTGGAACACCTGCTGCTGGCCCTGCAGATGATCCTGAACGGCATCAACAACTAC |
| | | AAGAACCCCAAGCTGACCGAGATGCTGACATTTAAGTTCTACATGCCTAAGAAAGCCACCGAGCTGAAG |
| | | CACCTGCAATGTCTGGAAGAAGAGCTGAAACCTCTGGAAGAGGTGCTGAATCTGGCTCAGTCAAAGAAC |
| | | TTCCACCTTAGACCTAGAGATCTGATCAGCAACATCAACGTGATCGTGCTGGAACTGAAGGGCAGCGAG |
| | | ACGACCTTCATGTGCGAGTACGCCGACGAGACAGCCACAATCGTGGAGTTCCTGAACAGATGGATCACC |
| | | TTCGCCCAGAGCATCATCTCCACCCTGACC |
| 413 | H7-767 LC | GAGATCGTGCTGACCCAGTCCCCAGGCACACTGAGCCTGAGCCCCGGCGAGCGGGCCACCCTGAGCTGT |
| | | AGAGCTAGCCAGAGCATCTCCAGCAGCTTCCTGGCCTGGTACCAGCAGAAACCTGGCCAGGCCCCTAGA |
| | | CTGCTGATCTACGACGCCTCTGATAGAGCTACAGGCATCCCCGACCGGTTCAGCGGCAGCGGATCTGGC |
| | | ACCGACTTCACCCTGACCATCAGCAGACTCGAGCCTGAAGATTTCGCCGTGTACTACTGCCAGCAATAC |
| | | TATGACTGGCCTCCTCTGTCTTTTGGCGGCGGAACAAAGGTGGAAATTAAGCGTACGGTGGCGGCGCCC |
| | | AGCGTGTTCATCTTCCCACCCAGCGACGAGCAGCTGAAGTCCGGCACAGCCAGCGTGGTGTGCCTGCTG |
| | | AACAACTTCTACCCCCGCGAGGCCAAGGTGCAGTGGAAGGTGGACAACGCCCTGCAGAGCGGCAACAGC |
| | | CAGGAAAGCGTGACCGAGCAGGACAGCAAGGACTCCACCTACAGCCTGAGCAGCACCCTGACCCTGAGC |
| | | AAGGCCGACTACGAGAAGCACAAGGTGTACGCCTGCGAAGTGACCCACCAGGGCCTGTCCAGCCCCGTG |
| | | ACCAAGAGCTTCAACCGGGGCGAGTGC |
| 414 | H7-632 HC | EVQLLESGGGLVQPGGSLRLSCAASGFTFKSYAMHWVRQAPGKGLEWVSAIVYSGGSTYYADSVKGRET |
| | CDRs solid | 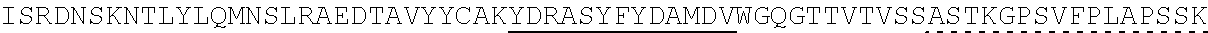 |
| | underlined | 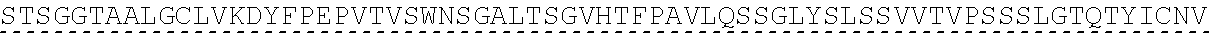 |
| | Constant | 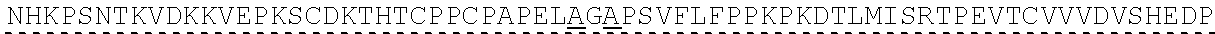 |
| | region dashed | 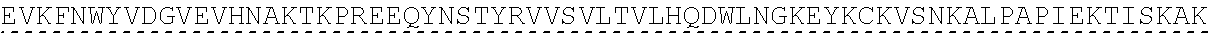 |
| | underlined | 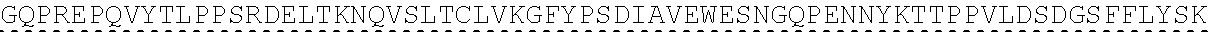 |
| | | 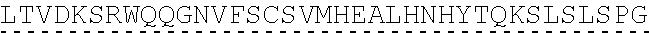 |
| 415 | H7-632 LC | EIVLTQSPGTLSLSPGERATLSCRASQSISSSFLAWYQQKPGQAPRLLIYDASDRATGIPDRESGSGSG |
| | CDRs solid | 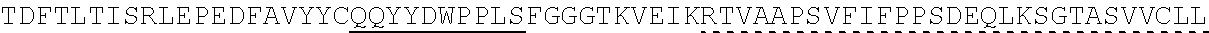 |
| | underlined | 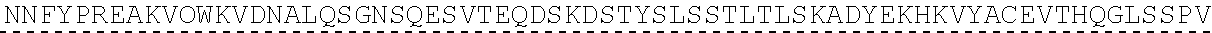 |
| | Constant | 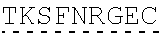 |
| | region dashed | |
| | underlined | |
| 416 | H7-632 VH | EVQLLESGGGLVQPGGSLRLSCAASGFTFKSYAMHWVRQAPGKGLEWVSAIVYSGGSTYYADSVKGRET |
| | | ISRDNSKNTLYLQMNSLRAEDTAVYYCAKYDRASYFYDAMDVWGQGTTVTVSS |
| 417 | H7-632 VL | EIVLTQSPGTLSLSPGERATLSCRASQSISSSFLAWYQQKPGQAPRLLIYDASDRATGIPDRESGSGSG |
| | | TDFTLTISRLEPEDFAVYYCQQYYDWPPLSFGGGTKVEIK |
| 418 | H7-632 HCDR1 | GFTFKSYAMH |
| 419 | H7-632 HCDR2 | AIVYSGGSTYYADSVKG |
| 420 | H7-632 HCDR3 | YDRASYFYDAMDV |
| 421 | H7-632 LCDR1 | RASQSISSSFLA |
| 422 | H7-632 LCDR2 | DASDRAT |
| 423 | H7-632 LCDR3 | QQYYDWPPLS |
| 424 | 2H7-hIgG4 HC | EVQLLESGGGLVQPGGSLRLSCAASGFTFKDYCMTWVRQAPGKGLEWVSAIVYSGGSTYYADSVKGRET |
| | | ISRDNSKNTLYLQMNNLRAEDTAVYYCAKYTRASYFYDAMDVWGQGTTVTVSSASTKGPSVFPLAPCSR |
| | | STSESTAALGCLVKDYFPEPVTVSWNSGALTSGVHTFPAVLQSSGLYSLSSVVTVPSSSLGTKTYTCNV |
| | | DHKPSNTKVDKRVESKYGPPCPPCPAPEFLGGPSVFLFPPKPKDTLMISRTPEVTCVVVDVSQEDPEVQ |
| | | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLG |
| 425 | 2H7-hkappa LC | EIVLTQSPGTLSLSPGERATLSCRASQSIGKSFLAWYQQKPGQAPRLLIYDASTRAADIPARESGSGSG |
| | | TDFTLTISSLEPEDFAVYYCQQYYDWPPLSFGGGTKVEIKRTVAAPSVFIFPPSDEQLKSGTASVVCLL |
| | | NNFYPREAKVQWKVDNALQSGNSQESVTEQDSKDSTYSLSSTLTLSKADYEKHKVYACEVTHQGLSSPV |
| | | TKSENRGEC |
| 426 | C51E6-5-hIgG4 | QVQLVQSGSELKKPGASVKVSCKASGYSLYGTSMHWVRQAPGQGLEWMGYISPFTGRATYAQGFTGREV |
| | HC | FSLDTSVSTAYLQISSLKAEDTAVYYCARDYDYRYYYAMDYWGQGTTVTVSSASTKGPSVFPLAPCSRS |
| | | TSESTAALGCLVKDYFPEPVTVSWNSGALTSGVHTFPAVLQSSGLYSLSSVVTVPSSSLGTKTYTCNVD |
| | | HKPSNTKVDKRVESKYGPPCPPCPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDGSFFLYSRLTVD |
| | | KSRWQEGNVFSCSVMHEALHNHYTQKSLSLSLG |
| 427 | C51E6-5-hKappa | EIVLTQSPDFQSVTPKEKVTITCTASESVPPQFLHWYQQKPDQSPKLLIYASRERASGVPSRESGSGSG |
| | LC | TDFTLTINSLEAEDAATYYCHQFHRSPLTFGGGTKLEIKRTVAAPSVFIFPPSDEQLKSGTASVVCLLN |
| | | NFYPREAKVQWKVDNALQSGNSQESVTEQDSKDSTYSLSSTLTLSKADYEKHKVYACEVTHQGLSSPVT |
| | | KSENRGEC |
| 428 | A2-hIgG4 HC | DVQLVESGGGLVQPGGSLRLSCAASGFTFDISAMSWVRQAPGKGLEWVSTISGSAYSTYYADSVKGRET |
| | | ISRDNSKSTLYLQMNSLRAEDTAVYYCAREIFSDYWGLGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNT |
| | | KVDKRVESKYGPPCPPCPAPEFLGGPSVFLFPPKPKDTLMISRTPEVTCVVVDVSQEDPEVQENWYVDG |
| | | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLG |
| 429 | A2-hLambda LC | QSVLTQPPSASGTPGQRVTISCSGSTSNIGRESVYWYQQLPGTAPKLLIYSNVQRPSGAPNRESGSKSG |
| | | TSASLAISGLQSEDEADYYCGTWDDSLNGWVFGGGTKLTVLGQPKAAPSVTLFPPSSEELQANKATLVC |
| | | LISDFYPGAVTVAWKADSSPVKAGVETTTPSKQSNNKYAASSYLSLTPEQWKSHRSYSCQVTHEGSTVE |
| | | KTVAPTECS |
| 430 | 2H7-hIgG4-df- | EVQLLESGGGLVQPGGSLRLSCAASGFTFKDYCMTWVRQAPGKGLEWVSAIVYSGGSTYYADSVKGRFT |
| | hIL-2 | ISRDNSKNTLYLQMNNLRAEDTAVYYCAKYTRASYFYDAMDVWGQGTTVTVSSASTKGPSVFPLAPCSR |
| | (D20A/R38E) HC | STSESTAALGCLVKDYFPEPVTVSWNSGALTSGVHTFPAVLQSSGLYSLSSVVTVPSSSLGTKTYTCNV |
| | hIL-2 in | DHKPSNTKVDKRVESKYGPPCPPCPAPEFLGGPSVFLFPPKPKDTLMISRTPEVTCVVVDVSQEDPEVQ |
| | italics | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPTSSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFCQSIISTLT |
| 431 | H7-632-hIgG1- | EVQLLESGGGLVQPGGSLRLSCAASGFTFKSYAMHWVRQAPGKGLEWVSAIVYSGGSTYYADSVKGRFT |
| | LAGA-df-hIL-2 | ISRDNSKNTLYLQMNSLRAEDTAVYYCAKYDRASYFYDAMDVWGQGTTVTVSSASTKGPSVFPLAPSSK |
| | (T3A/C125A) HC | STSGGTAALGCLVKDYFPEPVTVSWNSGALTSGVHTFPAVLQSSGLYSLSSVVTVPSSSLGTQTYICNV |
| | | NHKPSNTKVDKKVEPKSCDKTHTCPPCPAPELAGAPSVELFPPKPKDTLMISRTPEVTCVVVDVSHEDP |
| | | EVKFNWYVDGVEVHNAKTKPREEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLDLQMILNGINNY |
| | | KNPKLTRMLTFKFYMPKKATELKHLQCLEEELKPLEEVLNLAQSKNFHLRPRDLISNINVIVLELKGSE |
| | | TTFMCEYADETATIVEFLNRWITFAQSIISTLT |
| 432 | C51E6-5-hIgG4- | QVQLVQSGSELKKPGASVKVSCKASGYSLYGTSMHWVRQAPGQGLEWMGYISPFTGRATYAQGFTGREV |
| | L6-hIL-2 | FSLDTSVSTAYLQISSLKAEDTAVYYCARDYDYRYYYAMDYWGQGTTVTVSSASTKGPSVEPLAPCSRS |
| | (D20A/R38E) HC | TSESTAALGCLVKDYFPEPVTVSWNSGALTSGVHTFPAVLQSSGLYSLSSVVTVPSSSLGTKTYTCNVD |
| | Linker in | HKPSNTKVDKRVESKYGPPCPPCPAPEFLGGPSVFLFPPKPKDTLMISRTPEVTCVVVDVSQEDPEVQF |
| | dashed | NWYVDGVEVHNAKTKPREEQFNSTYRVVSVLTVLHQDWLNGKEYKCKVSNKGLPSSIEKTISKAKGQPR |
| | underline | EPQVYTLPPSQEEMTKNQVSLTCLVKGFYPSDIAVEWESNGQPENNYKTTPPVLDSDGSFFLYSRLTVD |
| | hIL-2 in | KSRWQEGNVFSCSVMHEALHNHYTQKSLSLSLGK 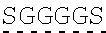 APTSSSTKKTQLQL |
| | italics | EHLLLALQMILNGINNYKNPKLTEMLTFKFYMPKKATELKHLQCLEEELKPLEEVLNLAQ |
| | | SKNFHLRPRDLISNINVIVLELKGSETTFMCEYADETATIVEFLNRWITFCQSIISTLT |
| 433 | A2-hIgG4-df- | DVQLVESGGGLVQPGGSLRLSCAASGFTFDISAMSWVRQAPGKGLEWVSTISGSAYSTYYADSVKGRET |
| | hIL-2 | ISRDNSKSTLYLQMNSLRAEDTAVYYCAREIFSDYWGLGTLVTVSSASTKGPSVFPLAPCSRSTSESTA |
| | (D20A/R38E) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLALQMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFCQSIISTLT |
| 434 | 1H3-hIgG4-df- | EVQLVESGGGLVQPGRSLKLSCAVSGFTFSDYAMAWVRQAPKKGLEWVATISYDGSRTYYRDSVKGRET |
| | hIL-2 | ISRDNAKITLYLQMDSLRSEDTATYYCARHGSGYFDYWGQGVMVTVSSASTKGPSVFPLAPCSRSTSES |
| | (D20A/R38E) HC | TAALGCLVKDYFPEPVTVSWNSGALTSGVHTFPAVLQSSGLYSLSSVVTVPSSSLGTKTYTCNVDHKPS |
| | hIL-2 in | NTKVDKRVESKYGPPCPPCPAPEFLGGPSVFLFPPKPKDTLMISRTPEVTCVVVDVSQEDPEVQENWYV |
| | italics | DGVEVHNAKTKPREEQFNSTYRVVSVLTVLHQDWLNGKEYKCKVSNKGLPSSIEKTISKAKGQPREPQV |
| | | YTLPPSQEEMTKNQVSLTCLVKGFYPSDIAVEWESNGQPENNYKTTPPVLDSDGSFFLYSRLTVDKSRW |
| | | QEGNVFSCSVMHEALHNHYTQKSLSLSLGKAPTSSSTKKTQLQLEHLLLALQMILNGINNYKNPKLTEM |
| | | LTFKFYMPKKATELKHLQCLEEELKPLEEVLNLAQSKNFHLRPRDLISNINVIVLELKGSETTEMCEYA |
| | | DETATIVEFLNRWITFCQSIISTLT |
| 435 | 2H7-hIgG4-df - | EVQLLESGGGLVQPGGSLRLSCAASGFTFKDYCMTWVRQAPGKGLEWVSAIVYSGGSTYYADSVKGRFT |
| | hIL-2 | ISRDNSKNTLYLQMNNLRAEDTAVYYCAKYTRASYFYDAMDVWGQGTTVTVSSASTKGPSVEPLAPCSR |
| | (T3A/D20A/R38E | STSESTAALGCLVKDYFPEPVTVSWNSGALTSGVHTFPAVLQSSGLYSLSSVVTVPSSSLGTKTYTCNV |
| | /C125A) HC | DHKPSNTKVDKRVESKYGPPCPPCPAPEFLGGPSVELFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFAQSIISTLT |
| 436 | hPD-1 | CCAGGATGGTTCTTAGACTCCCCAGACAGGCCCTGGAACCCCCCCACCTTCTCCCCAGCCCTGCTCGTG |
| | extracellular | GTGACCGAAGGGGACAACGCCACCTTCACCTGCAGCTTCTCCAACACATCGGAGAGCTTCGTGCTAAAC |
| | domain | TGGTACCGCATGAGCCCCAGCAACCAGACGGACAAGCTGGCCGCCTTCCCCGAGGACCGCAGCCAGCCC |
| | | GGCCAGGACTGCCGCTTCCGTGTCACACAACTGCCCAACGGGCGTGACTTCCACATGAGCGTGGTCAGG |
| | | GCCCGGCGCAATGACAGCGGCACCTACCTCTGTGGGGCCATCTCCCTGGCCCCCAAGGCGCAGATCAAA |
| | | GAGAGCCTGCGGGCAGAGCTCAGGGTGACAGAGAGAAGGGCAGAAGTGCCCACAGCCCACCCCAGCCCC |
| | | TCACCCAGGCCAGCCGGCCAGTTCCAA |
| 437 | hPD-1 | PGWFLDSPDRPWNPPTESPALLVVTEGDNATFTCSESNTSESFVLNWYRMSPSNQTDKLAAFPEDRSQP |
| | extracellular | GQDCRFRVTQLPNGRDFHMSVVRARRNDSGTYLCGAISLAPKAQIKESLRAELRVTERRAEVPTAHPSP |
| | domain HC | SPRPAGQFQ |
| 438 | OMC.1.B6-hIgG4 | EVQLLESGGGLVQPGGSLRLSCAASGFTESSNYMSWVRQAPGKGLEWVSAISSSGGTIFYADSVKGRFT |
| | HC | ISRDNSKNTLYLQMNSLRAEDTAVYYCAKHKWNAVYYDGMDVWGQGTTVTVSSASTKGPSVFPLAPCSR |
| | | STSESTAALGCLVKDYFPEPVTVSWNSGALTSGVHTFPAVLQSSGLYSLSSVVTVPSSSLGTKTYTCNV |
| | | DHKPSNTKVDKRVESKYGPPCPPCPAPEFLGGPSVFLFPPKPKDTLMISRTPEVTCVVVDVSQEDPEVQ |
| | | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLG |
| 439 | OMC.1.B6- | QSVLTQPPSASGTPGQRVTISCSGSNSNIGRNLVNWYQQLPGTAPKLLIYTIDQRPSGVPDRESGSKSG |
| | hLambda LC | TSASLVISGLQSEDEADYYCAAWDGSLNAWVEGGGTKLTVLGQPKAAPSVTLFPPSSEELQANKATLVC |
| | | LISDFYPGAVTVAWKADSSPVKAGVETTTPSKQSNNKYAASSYLSLTPEQWKSHRSYSCQVTHEGSTVE |
| | | KTVAPTECS |
| 440 | OMC.2.C6-hIgG4 | EVQLLESGGGLVQPGGSLRLSCTASGFTESSYEMQWVRQAPGKGLEWVLGITSSSSHIFYADSVKGRET |
| | HC | VSRDNSKNTLYLQMNSLRAEDTAVYYCTKDLNSYYGLDVWGQGTTVTVSSASTKGPSVEPLAPCSRSTS |
| | | ESTAALGCLVKDYFPEPVTVSWNSGALTSGVHTFPAVLQSSGLYSLSSVVTVPSSSLGTKTYTCNVDHK |
| | | PSNTKVDKRVESKYGPPCPPCPAPEFLGGPSVELFPPKPKDTLMISRTPEVTCVVVDVSQEDPEVQENW |
| | | YVDGVEVHNAKTKPREEQFNSTYRVVSVLTVLHQDWLNGKEYKCKVSNKGLPSSIEKTISKAKGQPREP |
| | | QVYTLPPSQEEMTKNQVSLTCLVKGFYPSDIAVEWESNGQPENNYKTTPPVLDSDGSFFLYSRLTVDKS |
| | | RWQEGNVFSCSVMHEALHNHYTQKSLSLSLG |
| 441 | OMC.2.C6- | QSVMTQPPSASGTPGQRVTISCSGSTSNLGNNYVSWYQHLPGTAPKLLIYGNDQRPSGVPDRESGSKSG |
| | hLambda LC | TSASLAISGLQSDDEADYYCSSWDASLNVWVFGGGTKLTVLGQPKAAPSVTLFPPSSEELQANKATLVC |
| | | LISDFYPGAVTVAWKADSSPVKAGVETTTPSKQSNNKYAASSYLSLTPEQWKSHRSYSCQVTHEGSTVE |
| | | KTVAPTECS |
| 442 | OMC.1.D6-hIgG4 | EVQLLESGGGLVQPGGSLRLSCAASGFTFSDYYMSWVRQAPGKGLEWVSAISSSGGTIFYADSVKGRFI |
| | HC | ISRDNSKNTLYLQMNSLRAEDTAVYYCAKHKWNDVYYDAMDVWGQGTTVTVSSASTKGPSVEPLAPCSR |
| | | STSESTAALGCLVKDYFPEPVTVSWNSGALTSGVHTFPAVLQSSGLYSLSSVVTVPSSSLGTKTYTCNV |
| | | DHKPSNTKVDKRVESKYGPPCPPCPAPEFLGGPSVFLFPPKPKDTLMISRTPEVTCVVVDVSQEDPEVQ |
| | | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLG |
| 443 | OMC.1.D6- | QSVLTQPPSASGTPGQRVTISCSGSNSNIGRNLVNWYQQLPGTAPKLLIYTVDQRPSGVPDRESGSKSG |
| | hLambda LC | TSASLAISGLASEDEADYYCAAWDSSLNSWVFGGGTKLTVLGQPKAAPSVTLFPPSSEELQANKATLVC |
| | | LISDFYPGAVTVAWKADSSPVKAGVETTTPSKQSNNKYAASSYLSLTPEQWKSHRSYSCQVTHEGSTVE |
| | | KTVAPTECS |
| 444 | D12-hIgG4 HC | DVQLVESGGGLVQPGGSLRLSCAASGFTFDISAMSWVRQAPGKGLEWVSTISGSAYSTYYADSVKGRFT |
| | | ISRDNSKSTLYLQMNSLRAEDTAVYYCAREIFSDYWGLGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNT |
| | | KVDKRVESKYGPPCPPCPAPEFLGGPSVFLFPPKPKDTLMISRTPEVTCVVVDVSQEDPEVQFNWYVDG |
| | | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLG |
| 445 | D12-hLambda LC | QSVLTQPPSASGTPGQRVTISCSGNTSNIGRESVYWYQQLPGTAPKLLIYSNVQRPSGVPDRESGSKSG |
| | | TSASLAISGLQSEDEADYYCGTWDDSLNGWVFGGGTKLTVLGQPKAAPSVTLEPPSSEELQANKATLVC |
| | | LISDFYPGAVTVAWKADSSPVKAGVETTTPSKQSNNKYAASSYLSLTPEQWKSHRSYSCQVTHEGSTVE |
| | | KTVAPTECS |
| 446 | G12-hIgG4 HC | DSLVESGGGLVQPGGSLRLSCAASGFTFDISAMSWVRQAPGKGLEWVSTISGSAYSTYYADSVKGRFTI |
| | | SRDNSKSTLYLQMNSLRAEDTAVYYCAREIFSDYWGLGTLVTVSSASTKGPSVFPLAPCSRSTSESTAA |
| | | LGCLVKDYFPEPVTVSWNSGALTSGVHTFPAVLQSSGLYSLSSVVTVPSSSLGTKTYTCNVDHKPSNTK |
| | | VDKRVESKYGPPCPPCPAPEFLGGPSVFLFPPKPKDTLMISRTPEVTCVVVDVSQEDPEVQFNWYVDGV |
| | | EVHNAKTKPREEQFNSTYRVVSVLTVLHQDWLNGKEYKCKVSNKGLPSSIEKTISKAKGQPREPQVYTL |
| | | PPSQEEMTKNQVSLTCLVKGFYPSDIAVEWESNGQPENNYKTTPPVLDSDGSFFLYSRLTVDKSRWQEG |
| | | NVFSCSVMHEALHNHYTQKSLSLSLG |
| 447 | G12-hLambda LC | QSVLTQPPSASGTPGQRVTISCSGSTSNIGRESVYWYQQLPGTAPKLLIYLNSQRPSGVPDRESGSKSG |
| | | TSASLAISGLQSEDVADYYCGTWDDSLNGWVFGGGTKLTVLGQPKAAPSVTLEPPSSEELQANKATLVC |
| | | LISDFYPGAVTVAWKADSSPVKAGVETTTPSKQSNNKYAASSYLSLTPEQWKSHRSYSCQVTHEGSTVE |
| | | KTVAPTECS |
| 448 | pCMV6-hygro- | aacaaaatattaacgcttacaatttccattcgccattcaggctgcgcaactgttgggaagggcgatcgg |
| | HA-cyno-PD-1 | tgcgggcctcttcgctattacgccagctggcgaaagggggatgtgctgcaaggcgattaagttgggtaa |
| | (1-185) | cgccagggttttcccagtcacgacgttgtaaaacgacggccagtgccaagctgatctatacattgaatc |
| | | aatattggcaattagccatattagtcattggttatatagcataaatcaatattggctattggccattgc |
| | | atacgttgtatctatatcataatatgtacatttatattggctcatgtccaatatgaccgccatgttgac |
| | | attgattattgactagttattaatagtaatcaattacggggtcattagttcatagcccatatatggagt |
| | | tccgcgttacataacttacggtaaatggcccgcctggctgaccgcccaacgacccccgcccattgacgt |
| | | caataatgacgtatgttcccatagtaacgccaatagggactttccattgacgtcaatgggtggagtatt |
| | | tacggtaaactgcccacttggcagtacatcaagtgtatcatatgccaagtccgccccctattgacgtca |
| | | atgacggtaaatggcccgcctggcattatgcccagtacatgaccttacgggactttcctacttggcagt |
| | | acatctacgtattagtcatcgctattaccatggtgatgcggttttggcagtacaccaatgggcgtggat |
| | | agcggtttgactcacggggatttccaagtctccaccccattgacgtcaatgggagtttgttttggcacc |
| | | aaaatcaacgggactttccaaaatgtcgtaataaccccgccccgttgacgcaaatgggggtaggcgtg |
| | | tacggtgggaggtctatataagcagagctcgtttagtgaaccgtcagaattttgtaatacgactcacta |
| | | tagggcggccgggaattcgtcgactggatccggtaccgaggagatctgccgccgcgatcgccggcgcgc |
| | | cagatctcaagcttatggacatgcgggtgccagcacaacttctcggattactattgttatggctgcgag |
| | | gtgcgcgctgttatccttacgacgtgcctgactacgccccaggatggttcttagagtccccagacaggc |
| | | cctggaacgcccccaccttctccccagccctgctcctggtgaccgaaggggacaacgccaccttcacct |
| | | gcagcttctccaacgcatcggagagcttcgtgctaaactggtacaggatgagccccagcaaccagacgg |
| | | acaagctggccgccttccccgaggaccgcagccagcccggccaggactgccgcttccgtgtcacacgcc |
| | | tgcccaacgggcgtgacttccacatgagcgtggtcagggcccggcgcaatgacagcggcacctacctct |
| | | gtggggccatctccctggcccccaaggcgcagatcaaagagagcctgcgggcagagctcagggtgacag |
| | | agagaagggcagaagtgcccacagcccaccccagcccctcacccaggccagccggccagttccaagccc |
| | | tggtggttggtgtcgtgggcggcctgctgggcagcctggtgctgctagtctgggtcctggccgtcatct |
| | | gctcccgcgccgcacaagggacaatagaagccaggcgcacctgacgcgttaagcggccgcactcgaggt |
| | | ttaaacggccggccgcggtcatagctgtttcctgaacagatcccgggggcatccctgtgacccctccc |
| | | cagtgcctctcctggccctggaagttgccactccagtgcccaccagccttgtcctaataaaattaagtt |
| | | gcatcattttgtctgactaggtgtccttctataatattatggggtggaggggggtggtatggagcaagg |
| | | ggcaagttgggaagacaacctgtagggcctgcggggtctattgggaaccaagctggagtgcagtggcac |
| | | aatcttggctcactgcaatctccgcctcctgggttcaagcgattctcctgcctcagcctcccgagttgt |
| | | tgggattccaggcatgcatgaccaggctcagctaattttttttttttggtagagacggggtttcacca |
| | | tattggccaggctggtctccaactcctaatctcaggtgatctacccaccttggcctcccaaattgctgg |
| | | gattacaggcgtgaaccactgctcccttccctgtccttctgattttaaaataactataccagcaggagg |
| | | acgtccagacacagcataggctacctggccatgcccaaccggtgggacatttgagttgcttgcttggca |
| | | ctgtcctctcatgcgttgggtccactcagtagatgcctgttgaattgggtacgcggccagcttggctgt |
| | | ggaatgtgtgtcagttagggtgtggaaagtccccaggctccccagcaggcagaagtatgcaaagcatgc |
| | | atctcaattagtcagcaaccaggtgtggaaagtccccaggctccccagcaggcagaagtatgcaaagca |
| | | tgcatctcaattagtcagcaaccatagtcccgcccctaactccgcccatcccgcccctaactccgccca |
| | | gttccgcccattctccgccccatggctgactaattttttttatttatgcagaggccgaggccgcctcgg |
| | | cctctgagctattccagaagtagtgaggaggcttttttggaggcctaggcttttgcaaaaagctcccgg |
| | | gagcttgtatatccattttcggatctgatcaagagacacgtacgaccatgaaaaagcctgaactcaccg |
| | | cgacgtctgttgagaagtttctgatcgaaaagttcgacagcgtctccgacctgatgcagctctcggagg |
| | | gcgaagaatctcgtgctttcagcttcgatgtaggagggcgtggatatgtcctgcgggtaaatagctgcg |
| | | ccgatggtttctacaaagatcgttatgtttatcggcactttgcatcggccgcgctcccgattccggaag |
| | | tgcttgacattggggaatttagcgagagcctgacctattgcatctcccgccgtgcacagggtgtcacgt |
| | | tgcaagacctgcctgaaaccgaactgcccgctgttctgcaaccggtcgcggaggccatggatgcaatcg |
| | | ctgcggccgatcttagccagacgagcgggttcggcccattcggaccgcaaggaatcggtcaatacacta |
| | | catggcgtgatttcatatgcgcgattgctgatccccatgtgtatcactggcaaactgtgatggacgaca |
| | | ccgtcagtgcgtccgtcgcgcaggctctcgatgagctgatgctttgggccgaggactgccccgaagtcc |
| | | ggcacctcgtgcacgcggatttcggctccaacaatgtcctgacggacaatggccgcataacagcggtca |
| | | ttgactggagcgaggcgatgttcggggattcccaatacgaggtcgccaacatcttcttctggaggccgt |
| | | ggttggcttgtatggagcagcagacgcgctacttcgagcggaggcatccggagcttgcaggatcgccgc |
| | | ggctccgggcgtatatgctccgcattggtcttgaccaactctatcagagcttggttgacggcaatttcg |
| | | atgatgcagcttgggcgcagggtcgatgcgacgcaatcgtccgatccggagccgggactgtcgggcgta |
| | | cacaaatcgcccgcagaagcgcggccgtctggaccgatggctgtgtagaagtactcgccgatagtggaa |
| | | accgacgccccagcactcgtccgagggcaaaggaatagctgcagcgggactctggggttcgaaatgacc |
| | | gaccaagcgacgcccaacctgccatcacgagatttcgattccaccgccgccttctatgaaaggttgggc |
| | | ttcggaatcgttttccgggacgccggctggatgatcctccagcgcggggatctcatgctggagttcttc |
| | | gcccaccccaacttgtttattgcagcttataatggttacaaataaagcaatagcatcacaaatttcaca |
| | | aataaagcatttttttcactgcattctagttgtggtttgtccaaactcatcaatgtatcttatcatgtc |
| | | tgtataccgtcgacctctagctagagcttggcgtaatcatggtcatagctgtttcctgtgtgaaattgt |
| | | tatccgctcacaattccacacaacatacgagccggaagcataaagtgtaaagcctggggtgcctaatga |
| | | gtgagctaactcacattaattgcgttgcgctcactgcccgctttccagtcgggaaacctgtcgtgccag |
| | | ctgcattaatgaatcggccaacgcgcggggagaggcggtttgcgtattgggcgctcttccgcttcctcg |
| | | ctcactgactcgctgcgctcggtcgttcggctgcggcgagcggtatcagctcactcaaaggcggtaata |
| | | cggttatccacagaatcaggggataacgcaggaaagaacatgtgagcaaaaggccagcaaaaggccagg |
| | | aaccgtaaaaaggccgcgttgctggcgtttttccataggctccgcccccctgacgagcatcacaaaaat |
| | | cgacgctcaagtcagaggtggcgaaacccgacaggactataaagataccaggcgtttccccctggaagc |
| | | tccctcgtgcgctctcctgttccgaccctgccgcttaccggatacctgtccgcctttctcccttcggga |
| | | agcgtggcgctttctcatagctcacgctgtaggtatctcagttcggtgtaggtcgttcgctccaagctg |
| | | ggctgtgtgcacgaaccccccgttcagcccgaccgctgcgccttatccggtaactatcgtcttgagtcc |
| | | aacccggtaagacacgacttatcgccactggcagcagccactggtaacaggattagcagagcgaggtat |
| | | gtaggcggtgctacagagttcttgaagtggtggcctaactacggctacactagaagaacagtatttggt |
| | | atctgcgctctgctgaagccagttaccttcggaaaaagagttggtagctcttgatccggcaaacaaacc |
| | | accgctggtagcggtggtttttttgtttgcaagcagcagattacgcgcagaaaaaaaggatctcaagaa |
| | | gatcctttgatcttttctacggggtctgacgctcagtggaacgaaaactcacgttaagggattttggtc |
| | | atgagattatcaaaaaggatcttcacctagatccttttaaattaaaaatgaagttttaaatcaatctaa |
| | | agtatatatgagtaaacttggtctgacagttaccaatgcttaatcagtgaggcacctatctcagcgatc |
| | | tgtctatttcgttcatccatagttgcctgactccccgtcgtgtagataactacgatacgggagggctta |
| | | ccatctggccccagtgctgcaatgataccgcgagacccacgctcaccggctccagatttatcagcaata |
| | | aaccagccagccggaagggccgagcgcagaagtggtcctgcaactttatccgcctccatccagtctatt |
| | | aattgttgccgggaagctagagtaagtagttcgccagttaatagtttgcgcaacgttgttgccattgct |
| | | acaggcatcgtggtgtcacgctcgtcgtttggtatggcttcattcagctccggttcccaacgatcaagg |
| | | cgagttacatgatcccccatgttgtgcaaaaaagcggttagctccttcggtcctccgatcgttgtcaga |
| | | agtaagttggccgcagtgttatcactcatggttatggcagcactgcataattctcttactgtcatgcca |
| | | tccgtaagatgcttttctgtgactggtgagtactcaaccaagtcattctgagaatagtgtatgcggcga |
| | | ccgagttgctcttgcccggcgtcaatacgggataataccgcgccacatagcagaactttaaaagtgctc |
| | | atcattggaaaacgttcttcggggcgaaaactctcaaggatcttaccgctgttgagatccagttcgatg |
| | | taacccactcgtgcacccaactgatcttcagcatcttttactttcaccagcgtttctgggtgagcaaaa |
| | | acaggaaggcaaaatgccgcaaaaaagggaataagggcgacacggaaatgttgaatactcatactcttc |
| | | ctttttcaatattattgaagcatttatcagggttattgtctcatgagcggatacatatttgaatgtatt |
| | | tagaaaaataaacaaataggggttccgcgcacatttccccgaaaagtgccacctgacgcgccctgtagc |
| | | ggcgcattaagcgcggcgggtgtggtggttacgcgcagcgtgaccgctacacttgccagcgccctagcg |
| | | cccgctcctttcgctttcttcccttcctttctcgccacgttcgccggctttccccgtcaagctctaaat |
| | | cgggggctccctttagggttccgatttagtgctttacggcacctcgaccccaaaaaacttgattagggt |
| | | gatggttcacgtagtgggccatcgccctgatagacggtttttcgccctttgacgttggagtccacgttc |
| | | tttaatagtggactcttgttccaaactggaacaacactcaaccctatctcggtctattcttttgattta |
| | | taagggattttgccgatttcggcctattggttaaaaaatgagctgatttaacaaaaatttaacgcgaat |
| | | ttt |
| 449 | Abz1mod-hIgG4 | QVQLVQSGSELKKPGASVKVSCKASGYTFTSYAMHWVRQAPGQGLEWMGYISPFTGRATYAQGFTGREV |
| | HC | FSLDTSVSTAYLQISSLKAEDTAVYYCARDYDYRYYYAMDYWGQGTTVTVSSASTKGPSVFPLAPCSRS |
| | | TSESTAALGCLVKDYFPEPVTVSWNSGALTSGVHTFPAVLQSSGLYSLSSVVTVPSSSLGTKTYTCNVD |
| | | HKPSNTKVDKRVESKYGPPCPPCPAPEFLGGPSVFLFPPKPKDTLMISRTPEVTCVVVDVSQEDPEVQF |
| | | NWYVDGVEVHNAKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDGSFFLYSRLTVD |
| | | KSRWQEGNVFSCSVMHEALHNHYTQKSLSLSLG |
| 450 | Abz1mod-hKappa | EIVLTQSPDFQSVTPKEKVTITCRASQSIPPQFLHWYQQKPDQSPKLLIKAASQRASGVPSRESGSGSG |
| | LC | TDFTLTINSLEAEDAATYYCHQFHSSPLTFGGGTKLEIKRTVAAPSVFIFPPSDEQLKSGTASVVCLLN |
| | | NFYPREAKVQWKVDNALQSGNSQESVTEQDSKDSTYSLSSTLTLSKADYEKHKVYACEVTHQGLSSPVT |
| | | KSFNRGEC |
| 451 | Anti-hPD-1 #1- | QVQLVESGGGVVQPGRSLRLDCKASGITFSNSGMHWVRQAPGKGLEWVAVIWYDGSKRYYADSVKGRET |
| | hIgG4-L6-hIL-2 | ISRDNSKNTLFLQMNSLRAEDTAVYYCATNDDYWGQGTLVTVSSASTKGPSVEPLAPCSRSTSESTAAL |
| | (D20A/R38E) HC | GCLVKDYFPEPVTVSWNSGALTSGVHTFPAVLOSSGLYSLSSVVTVPSSSLGTKTYTCNVDHKPSNTKV |
| | Linker in | DKRVESKYGPPCPPCPAPEFLGGPSVELFPPKPKDTLMISRTPEVTCVVVDVSQEDPEVQFNWYVDGVE |
| | dashed | VHNAKTKPREEQFNSTYRVVSVLTVLHQDWLNGKEYKCKVSNKGLPSSIEKTISKAKGQPREPQVYTLP |
| | underline | PSQEEMTKNQVSLTCLVKGFYPSDIAVEWESNGQPENNYKTTPPVLDSDGSFFLYSRLTVDKSRWQEGN |
| | hIL-2 in | VFSCSVMHEALHNHYTQKSLSLSLGK 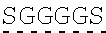 APTSSSTKKTQLQLEHLLLAL |
| | italics | QMILNGINNYKNPKLTEMLTFKFYMPKKATELKHLOCLEEELKPLEEVLNLAQSKNFHLRPRD |
| | | LISNINVIVLELKGSETTEMCEYADETATIVEFLNRWITFCQSIISTLT |
| 452 | Anti-hPD-1 #1- | EIVLTQSPATLSLSPGERATLSCRASQSVSSYLAWYQQKPGQAPRLLIYDASNRATGIPARESGSGSGT |
| | hKappa LC | DFTLTISSLEPEDFAVYYCQQSSNWPRTFGQGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPVTK |
| | | SENRGEC |
| 453 | OMC.1.B6- | EVQLLESGGGLVQPGGSLRLSCAASGFTESSNYMSWVRQAPGKGLEWVSAISSSGGTIFYADSVKGRFT |
| | hIgG4-L6-hIL-2 | ISRDNSKNTLYLQMNSLRAEDTAVYYCAKHKWNAVYYDGMDVWGQGTTVTVSSASTKGPSVEPLAPCSR |
| | (D20A/R38E) HC | STSESTAALGCLVKDYFPEPVTVSWNSGALTSGVHTFPAVLQSSGLYSLSSVVTVPSSSLGTKTYTCNV |
| | Linker in | DHKPSNTKVDKRVESKYGPPCPPCPAPEFLGGPSVELFPPKPKDTLMISRTPEVTCVVVDVSQEDPEVQ |
| | dashed | FNWYVDGVEVHNAKTKPREEQFNSTYRVVSVLTVLHQDWLNGKEYKCKVSNKGLPSSIEKTISKAKGQP |
| | underline | REPQVYTLPPSQEEMTKNQVSLTCLVKGFYPSDIAVEWESNGQPENNYKTTPPVLDSDGSFFLYSRLTV |
| | hIL-2 in | DKSRWQEGNVFSCSVMHEALHNHYTQKSLSLSLGK 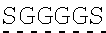 APTSSSTKKTQLQL |
| | italics | EHLLLALQMILNGINNYKNPKLTEMLTFKFYMPKKATELKHLQCLEEELKPLEEVLNLAQSKNF |
| | | HLRPRDLISNINVIVLELKGSETTFMCEYADETATIVEFLNRWITFCQSIISTLT |
| 454 | OMC.2.C6- | EVQLLESGGGLVQPGGSLRLSCTASGFTESSYEMQWVRQAPGKGLEWVLGITSSSSHIFYADSVKGRFT |
| | hIgG4-L6-hIL-2 | VSRDNSKNTLYLQMNSLRAEDTAVYYCTKDLNSYYGLDVWGQGTTVTVSSASTKGPSVFPLAPCSRSTS |
| | (D20A/R38E) HC | ESTAALGCLVKDYFPEPVTVSWNSGALTSGVHTFPAVLQSSGLYSLSSVVTVPSSSLGTKTYTCNVDHK |
| | Linker in | PSNTKVDKRVESKYGPPCPPCPAPEFLGGPSVFLFPPKPKDTLMISRTPEVTCVVVDVSQEDPEVQENW |
| | dashed | YVDGVEVHNAKTKPREEQFNSTYRVVSVLTVLHQDWLNGKEYKCKVSNKGLPSSIEKTISKAKGQPREP |
| | underline | QVYTLPPSQEEMTKNQVSLTCLVKGFYPSDIAVEWESNGQPENNYKTTPPVLDSDGSFFLYSRLTVDKS |
| | hIL-2 in | RWQEGNVFSCSVMHEALHNHYTQKSLSLSLGK 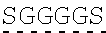 APTSSST |
| | italics | KKTQLQLEHLLLALQMILNGINNYKNPKLTEMLTFKFYMPKKATELKHLQCLEEELKPLEEVLNLAQS |
| | | KNFHLRPRDLISNINVIVLELKGSETTFMCEYADETATIVEFLNRWITFCQSIISTLT |
| 455 | OMC.1.D6- | EVQLLESGGGLVQPGGSLRLSCAASGFTESDYYMSWVRQAPGKGLEWVSAISSSGGTIFYADSVKGRFI |
| | hIgG4-L6-hIL-2 | ISRDNSKNTLYLQMNSLRAEDTAVYYCAKHKWNDVYYDAMDVWGQGTTVTVSSASTKGPSVFPLAPCSR |
| | (D20A/R38E) HC | STSESTAALGCLVKDYFPEPVTVSWNSGALTSGVHTFPAVLQSSGLYSLSSVVTVPSSSLGTKTYTCNV |
| | Linker in | DHKPSNTKVDKRVESKYGPPCPPCPAPEFLGGPSVELFPPKPKDTLMISRTPEVTCVVVDVSQEDPEVQ |
| | dashed | FNWYVDGVEVHNAKTKPREEQFNSTYRVVSVLTVLHQDWLNGKEYKCKVSNKGLPSSIEKTISKAKGQP |
| | underline | REPQVYTLPPSQEEMTKNQVSLTCLVKGFYPSDIAVEWESNGQPENNYKTTPPVLDSDGSFFLYSRLTV |
| | hIL-2 in | DKSRWQEGNVFSCSVMHEALHNHYTQKSLSLSLGK 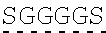 APTSSSTKKTQLQLE |
| | italics | HLLLALQMILNGINNYKNPKLTEMLTFKFYMPKKATELKHLQCLEEELKPLEEVLNLAQSKN |
| | | FHLRPRDLISNINVIVLELKGSETTEMCEYADETATIVEFLNRWITFCQSIISTLT |
| 456 | D12-hIgG4-df- | DVQLVESGGGLVQPGGSLRLSCAASGFTFDISAMSWVRQAPGKGLEWVSTISGSAYSTYYADSVKGRFT |
| | hIL-2 | ISRDNSKSTLYLQMNSLRAEDTAVYYCAREIFSDYWGLGTLVTVSSASTKGPSVFPLAPCSRSTSESTA |
| | (D20A/R38E) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLALQMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFCQSIISTLT |
| 457 | G12-hIgG4-df- | DSLVESGGGLVQPGGSLRLSCAASGFTFDISAMSWVRQAPGKGLEWVSTISGSAYSTYYADSVKGRFTI |
| | hIL-2 | SRDNSKSTLYLQMNSLRAEDTAVYYCAREIFSDYWGLGTLVTVSSASTKGPSVFPLAPCSRSTSESTAA |
| | (D20A/R38E) HC | LGCLVKDYFPEPVTVSWNSGALTSGVHTFPAVLQSSGLYSLSSVVTVPSSSLGTKTYTCNVDHKPSNTK |
| | hIL-2 in | VDKRVESKYGPPCPPCPAPEFLGGPSVELFPPKPKDTLMISRTPEVTCVVVDVSQEDPEVQFNWYVDGV |
| | italics | EVHNAKTKPREEQFNSTYRVVSVLTVLHQDWLNGKEYKCKVSNKGLPSSIEKTISKAKGQPREPQVYTL |
| | | PPSQEEMTKNQVSLTCLVKGFYPSDIAVEWESNGQPENNYKTTPPVLDSDGSFFLYSRLTVDKSRWQEG |
| | | NVFSCSVMHEALHNHYTQKSLSLSLGKAPTSSSTKKTQLQLEHLLLALQMILNGINNYKNPKLTEMLTF |
| | | KFYMPKKATELKHLQCLEEELKPLEEVLNLAQSKNFHLRPRDLISNINVIVLELKGSETTEMCEYADET |
| | | ATIVEFLNRWITFCQSIISTLT |
| 458 | 2H7-hIgG4-LE | EVQLLESGGGLVQPGGSLRLSCAASGFTFKDYCMTWVRQAPGKGLEWVSAIVYSGGSTYYADSVKGRET |
| | HC | ISRDNSKNTLYLQMNNLRAEDTAVYYCAKYTRASYFYDAMDVWGQGTTVTVSSASTKGPSVFPLAPCSR |
| | | STSESTAALGCLVKDYFPEPVTVSWNSGALTSGVHTFPAVLQSSGLYSLSSVVTVPSSSLGTKTYTCNV |
| | | DHKPSNTKVDKRVESKYGPPCPPCPAPEFEGGPSVFLFPPKPKDTLMISRTPEVTCVVVDVSQEDPEVQ |
| | | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLG |
| 459 | 2H7-hIgG4-LAGA | EVQLLESGGGLVQPGGSLRLSCAASGFTFKDYCMTWVRQAPGKGLEWVSAIVYSGGSTYYADSVKGRFT |
| | HC | ISRDNSKNTLYLQMNNLRAEDTAVYYCAKYTRASYFYDAMDVWGQGTTVTVSSASTKGPSVFPLAPCSR |
| | | STSESTAALGCLVKDYFPEPVTVSWNSGALTSGVHTFPAVLQSSGLYSLSSVVTVPSSSLGTKTYTCNV |
| | | DHKPSNTKVDKRVESKYGPPCPPCPAPEFAGAPSVELFPPKPKDTLMISRTPEVTCVVVDVSQEDPEVQ |
| | | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLG |
| 460 | OMC.2-A3- | EVQLLESGGCLVQPGGSLRLSCAASGFTFSDYYMSWVRQAPGKGLEWVSAISSSGGTIFYADSVKGRFI |
| | hIgG4/A HC | ISRDNSKNTLYLQMNSLRAEDTAVYYCAKHKWNDVYYDAMDVWGQGTTVTVSSASTKGPSVEPLAPCSR |
| | | STSESTAALGCLVKDYFPEPVTVSWNSGALTSGVHTFPAVLQSSGLYSLSSVVTVPSSSLGTKTYTCNV |
| | | DHKPSNTKVDKRVESKYGPPCPPCPAPEFLGGPSVELFPPKPKDTLMISRTPEVTCVVVDVSQEDPEVQ |
| | | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLG |
| 461 | OMC476pH7- | DMQLVESGGGVVRPGESLRLSCTASGFTFSISAMSWVRQAPGKGLEWVSAISGTAYSTYYADSVRGRFT |
| | hIgG4 HC | ISRDNSKNTLYLQMNSLRAEDTAVYYCAKDNFFDYWGLGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNT |
| | | KVDKRVESKYGPPCPPCPAPEFLGGPSVELFPPKPKDTLMISRTPEVTCVVVDVSQEDPEVQENWYVDG |
| | | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLG |
| 462 | OMC476pB11.H7 | QSVMTQPPSASGTPGQRVTISCSGVTSNIGSNSVYWYQQLPGTAPKLLIYLNSQRPSGVPDRESGSKSG |
| | LC | TSASLAISGLQSEDEADYYCGTWDDSLNGWVFGGGTKLTVLGQPKAAPSVTLFPPSSEELQANKATLVC |
| | | LISDFYPGAVTVAWKADSSPVKAGVETTTPSKQSNNKYAASSYLSLTPEQWKSHRSYSCQVTHEGSTVE |
| | | KTVAPTECS |
| 463 | OMC476pB11- | DVQLVESGGGVVRPGESLRLSCTASGFTFSISAMSWVRQAPGKGLEWVSAISGTAYSTYYADSVRGRFT |
| | hIgG4 HC | ISRDNSKNTLYLQMNSLRAEDTAVYYCAKDNFFDYWGLGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNT |
| | | KVDKRVESKYGPPCPPCPAPEFLGGPSVFLFPPKPKDTLMISRTPEVTCVVVDVSQEDPEVQFNWYVDG |
| | | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLG |
| 464 | OMC476pG10- | DVQLVESGGGVVRPGGSLRLSCAASGFTFSIYAMSWVRQAPGEGLEWVSHISASGGSTYYADSVKGRFA |
| | hIgG4 HC | ISRDNSKNTLYLQMNSLRAEDTAVYYCTTNLGSDYWGLGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNT |
| | | KVDKRVESKYGPPCPPCPAPEFLGGPSVFLFPPKPKDTLMISRTPEVTCVVVDVSQEDPEVQFNWYVDG |
| | | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLG |
| 465 | OMC476pH10- | DVQLVESGGGVVRPGGSLRLSCAASGFTESIYAVSWVRQAPGEGLEWVSHISASGGSTYYADSVKGRFA |
| | hIgG4 HC | ISRDNSKNTLYLQMNSLRAEDTAVYYCTTNLGSDYWGLGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNT |
| | | KVDKRVESKYGPPCPPCPAPEFLGGPSVFLFPPKPKDTLMISRTPEVTCVVVDVSQEDPEVQENWYVDG |
| | | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLG |
| 466 | OMC476pG10.H10 | QSVLTQPPSASGTPGQRVTISCSGSYSDIGTNYVYWYQQLPGTAPKLLIFATDRRPSGVPDRESGSKSG |
| | LC | TSASLAISGLQSEDEADYYCGTWDDSLNVWVFGGGTKLTVLGQPKAAPSVTLFPPSSEELQANKATLVC |
| | | LISDFYPGAVTVAWKADSSPVKAGVETTTPSKQSNNKYAASSYLSLTPEQWKSHRSYSCQVTHEGSTVE |
| | | KTVAPTECS |
| 467 | OMC476pE4- | DVQLVESGGGVVRPGESLRLSCAASGFTESTDAMGWVRQAPGEGLEWVSLISGSGYSTYYADSVKGRFT |
| | hIgG4 HC | ISRDNSKNTLYLQMNSLTAEDTAVYYCAKNSLAFFDYWGLGTLVTVSSASTKGPSVFPLAPCSRSTSES |
| | | TAALGCLVKDYFPEPVTVSWNSGALTSGVHTFPAVLOSSGLYSLSSVVTVPSSSLGTKTYTCNVDHKPS |
| | | NTKVDKRVESKYGPPCPPCPAPEFLGGPSVFLFPPKPKDTLMISRTPEVTCVVVDVSQEDPEVQFNWYV |
| | | DGVEVHNAKTKPREEQFNSTYRVVSVLTVLHQDWLNGKEYKCKVSNKGLPSSIEKTISKAKGQPREPQV |
| | | YTLPPSQEEMTKNQVSLTCLVKGFYPSDIAVEWESNGQPENNYKTTPPVLDSDGSFFLYSRLTVDKSRW |
| | | QEGNVFSCSVMHEALHNHYTQKSLSLSLG |
| 468 | OMC476pE4 LC | QSVLTQPPSASGTPGQRVTISCSGGSSNIGRESVNWYQQLPGTAPKLLIYSTDRRPSGVPDRESGSKSG |
| | | TSASLAISGLQSEDEADYYCGTWDNDLNGWVFGGGTKLTVLGQPKAAPSVTLFPPSSEELQANKATLVC |
| | | LISDFYPGAVTVAWKADSSPVKAGVETTTPSKQSNNKYAASSYLSLTPEQWKSHRSYSCQVTHEGSTVE |
| | | KTVAPTECS |
| 469 | J110-hIgG1 HC | DVQLQESGPGLVKPSQSLSLTCTVTGHSITSDYAWNWIRQFPGDKLEWMGYISYSGYTTYNPSLKSRVS |
| | | ITRDTSKNQFFLQLNSVTTEDTATYFCARDLDYGPWFAYWGQGTLVTVSAASTKGPSVFPLAPSSKSTS |
| | | GGTAALGCLVKDYFPEPVTVSWNSGALTSGVHTFPAVLQSSGLYSLSSVVTVPSSSLGTQTYICNVNHK |
| | | PSNTKVDKKVEPKSCDKTHTCPPCPAPELLGGPSVELFPPKPKDTLMISRTPEVTCVVVDVSHEDPEVK |
| | | FNWYVDGVEVHNAKTKPREEQYNSTYRVVSVLTVLHQDWLNGKEYKCKVSNKALPAPIEKTISKAKGQP |
| | | REPQVYTLPPSRDELTKNQVSLTCLVKGFYPSDIAVEWESNGQPENNYKTTPPVLDSDGSFFLYSKLTV |
| | | DKSRWQQGNVFSCSVMHEALHNHYTQKSLSLSPGK |
| 470 | J110-hKappa | DIQMTQSPASLSASVGETVTLTCRASENIHNYLAWYQQKQGKSPQLLVYNVKTLADGVPSRESGSGSGT |
| | LC | QYSLKINSLOPEDFGSYYCQHFWSSPWTFGGGTKVEIKRTVAAPSVFIFPPSDEQLKSGTASVVCLLNN |
| | | FYPREAKVQWKVDNALQSGNSQESVTEQDSKDSTYSLSSTLTLSKADYEKHKVYACEVTHQGLSSPVTK |
| | | SFNRGEC |
| 471 | 2H7-hIgG1- | EVQLLESGGGLVQPGGSLRLSCAASGFTFKDYCMTWVRQAPGKGLEWVSAIVYSGGSTYYADSVKGRFT |
| | LAGA-df-hIL-2 | ISRDNSKNTLYLQMNNLRAEDTAVYYCAKYTRASYFYDAMDVWGQGTTVTVSSASTKGPSVFPLAPSSK |
| | (T3A/D20A/R38E/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLALOMILNGINNY |
| | | KNPKLTEMLTFKFYMPKKATELKHLQCLEEELKPLEEVLNLAQSKNFHLRPRDLISNINVIVLELKGSE |
| | | TTFMCEYADETATIVEFLNRWITFAQSIISTLT |
| 472 | 2H7-hIgG4-LE- | EVQLLESGGGLVQPGGSLRLSCAASGFTFKDYCMTWVRQAPGKGLEWVSAIVYSGGSTYYADSVKGRET |
| | df-hIL-2 | ISRDNSKNTLYLQMNNLRAEDTAVYYCAKYTRASYFYDAMDVWGQGTTVTVSSASTKGPSVFPLAPCSR |
| | (T3A/D20A/R38E | STSESTAALGCLVKDYFPEPVTVSWNSGALTSGVHTFPAVLQSSGLYSLSSVVTVPSSSLGTKTYTCNV |
| | /C125A) HC | DHKPSNTKVDKRVESKYGPPCPPCPAPEFEGGPSVELFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFAQSIISTLT |
| 473 | 2H7-hIgG1- | EVQLLESGGGLVQPGGSLRLSCAASGFTFKDYCMTWVRQAPGKGLEWVSAIVYSGGSTYYADSVKGRFT |
| | LAGA--df-hIL-2 | ISRDNSKNTLYLQMNNLRAEDTAVYYCAKYTRASYFYDAMDVWGQGTTVTVSSASTKGPSVFPLAPCSR |
| | (T3A/D20A/R38E | STSESTAALGCLVKDYFPEPVTVSWNSGALTSGVHTFPAVLQSSGLYSLSSVVTVPSSSLGTKTYTCNV |
| | /C125A) HC | DHKPSNTKVDKRVESKYGPPCPPCPAPEFAGAPSVELFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFAQSIISTLT |
| 474 | 2H7-hIgG1- | EVQLLESGGGLVQPGGSLRLSCAASGFTFKDYCMTWVRQAPGKGLEWVSAIVYSGGSTYYADSVKGRFT |
| | LAGA-df-hIL-2 | ISRDNSKNTLYLQMNNLRAEDTAVYYCAKYTRASYFYDAMDVWGQGTTVTVSSASTKGPSVFPLAPSSK |
| | (T3A/R38E/192K/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLDLQMILNGINNY |
| | | KNPKLTEMLTFKFYMPKKATELKHLQCLEEELKPLEEVLNLAQSKNFHLRPRDLISNINVKVLELKGSE |
| | | TTFMCEYADETATIVEFLNRWITFAQSIISTLT |
| 475 | 2H7-hIgG4-LE- | EVQLLESGGGLVQPGGSLRLSCAASGFTFKDYCMTWVRQAPGKGLEWVSAIVYSGGSTYYADSVKGRET |
| | df-hIL-2 | ISRDNSKNTLYLQMNNLRAEDTAVYYCAKYTRASYFYDAMDVWGQGTTVTVSSASTKGPSVFPLAPCSR |
| | (T3A/R38E/192K/ | STSESTAALGCLVKDYFPEPVTVSWNSGALTSGVHTFPAVLQSSGLYSLSSVVTVPSSSLGTKTYTCNV |
| | C125A) HC | DHKPSNTKVDKRVESKYGPPCPPCPAPEFEGGPSVFLFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLDLQMILNGINNYKNP |
| | | KLTEMLTFKFYMPKKATELKHLQCLEEELKPLEEVLNLAQSKNFHLRPRDLISNINVKVLELKGSETTE |
| | | MCEYADETATIVEFLNRWITFAQSIISTLT |
| 476 | 2H7-hIgG4- | EVQLLESGGGLVQPGGSLRLSCAASGFTFKDYCMTWVRQAPGKGLEWVSAIVYSGGSTYYADSVKGRFT |
| | LAGA-df-hIL-2 | ISRDNSKNTLYLQMNNLRAEDTAVYYCAKYTRASYFYDAMDVWGQGTTVTVSSASTKGPSVFPLAPCSR |
| | (T3A/R38E/192K/ | STSESTAALGCLVKDYFPEPVTVSWNSGALTSGVHTFPAVLQSSGLYSLSSVVTVPSSSLGTKTYTCNV |
| | C125A) HC | DHKPSNTKVDKRVESKYGPPCPPCPAPEFAGAPSVELFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLDLQMILNGINNYKNP |
| | | KLTEMLTFKFYMPKKATELKHLOCLEEELKPLEEVLNLAQSKNFHLRPRDLISNINVKVLELKGSETTE |
| | | MCEYADETATIVEFLNRWITFAQSIISTLT |
| 477 | 2H7-hIgG1- | EVQLLESGGGLVQPGGSLRLSCAASGFTFKDYCMTWVRQAPGKGLEWVSAIVYSGGSTYYADSVKGRFT |
| | LAGA-df-hIL-2 | ISRDNSKNTLYLQMNNLRAEDTAVYYCAKYTRASYFYDAMDVWGQGTTVTVSSASTKGPSVFPLAPSSK |
| | (T3A/R38E/D84K/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLDLQMILNGINNY |
| | | KNPKLTEMLTFKFYMPKKATELKHLQCLEEELKPLEEVLNLAQSKNFHLRPRKLISNINVIVLELKGSE |
| | | TTFMCEYADETATIVEFLNRWITFAQSIISTLT |
| 478 | 2H7-hIgG4-LE- | EVQLLESGGGLVQPGGSLRLSCAASGFTFKDYCMTWVRQAPGKGLEWVSAIVYSGGSTYYADSVKGRFT |
| | df-hIL-2 | ISRDNSKNTLYLQMNNLRAEDTAVYYCAKYTRASYFYDAMDVWGQGTTVTVSSASTKGPSVEPLAPCSR |
| | (T3A/R38E/D84K/ | STSESTAALGCLVKDYFPEPVTVSWNSGALTSGVHTFPAVLQSSGLYSLSSVVTVPSSSLGTKTYTCNV |
| | C125A) HC | DHKPSNTKVDKRVESKYGPPCPPCPAPEFEGGPSVFLFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLDLQMILNGINNYKNP |
| | | KLTEMLTFKFYMPKKATELKHLQCLEEELKPLEEVLNLAQSKNFHLRPRKLISNINVIVLELKGSETTE |
| | | MCEYADETATIVEFLNRWITFAQSIISTLT |
| 479 | 2H7-hIgG4- | EVQLLESGGGLVQPGGSLRLSCAASGFTFKDYCMTWVRQAPGKGLEWVSAIVYSGGSTYYADSVKGRFT |
| | LAGA-df-hIL-2 | ISRDNSKNTLYLQMNNLRAEDTAVYYCAKYTRASYFYDAMDVWGQGTTVTVSSASTKGPSVFPLAPCSR |
| | (T3A/R38E/D84K/ | STSESTAALGCLVKDYFPEPVTVSWNSGALTSGVHTFPAVLQSSGLYSLSSVVTVPSSSLGTKTYTCNV |
| | C125A) HC | DHKPSNTKVDKRVESKYGPPCPPCPAPEFAGAPSVELFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGOP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLDLQMILNGINNYKNP |
| | | KLTEMLTFKFYMPKKATELKHLQCLEEELKPLEEVLNLAQSKNFHLRPRKLISNINVIVLELKGSETTE |
| | | MCEYADETATIVEFLNRWITFAQSIISTLT |
| 480 | 1H3-hIgG4-df- | EVOLVESGGGLVQPGRSLKLSCAVSGFTESDYAMAWVRQAPKKGLEWVATISYDGSRTYYRDSVKGRET |
| | hIL-2 (WT) HC | ISRDNAKITLYLQMDSLRSEDTATYYCARHGSGYFDYWGQGVMVTVSSASTKGPSVFPLAPCSRSTSES |
| | hIL-2 in | TAALGCLVKDYFPEPVTVSWNSGALTSGVHTFPAVLQSSGLYSLSSVVTVPSSSLGTKTYTCNVDHKPS |
| | italics | NTKVDKRVESKYGPPCPPCPAPEFLGGPSVELFPPKPKDTLMISRTPEVTCVVVDVSQEDPEVQFNWYV |
| | | DGVEVHNAKTKPREEQFNSTYRVVSVLTVLHQDWLNGKEYKCKVSNKGLPSSIEKTISKAKGQPREPQV |
| | | YTLPPSQEEMTKNQVSLTCLVKGFYPSDIAVEWESNGQPENNYKTTPPVLDSDGSFFLYSRLTVDKSRW |
| | | QEGNVFSCSVMHEALHNHYTQKSLSLSLGKAPTSSSTKKTQLQLEHLLLDLQMILNGINNYKNPKLTRM |
| | | LTFKFYMPKKATELKHLQCLEEELKPLEEVLNLAQSKNFHLRPRDLISNINVIVLELKGSETTEMCEYA |
| | | DETATIVEFLNRWITFCQSIISTLT |
| 481 | 1H3-hIgG4-L6- | EVQLVESGGGLVQPGRSLKLSCAVSGFTFSDYAMAWVRQAPKKGLEWVATISYDGSRTYYRDSVKGRFT |
| | hIL-2 (WT) HC | ISRDNAKITLYLOMDSLRSEDTATYYCARHGSGYFDYWGQGVMVTVSSASTKGPSVFPLAPCSRSTSES |
| | Linker in | TAALGCLVKDYFPEPVTVSWNSGALTSGVHTFPAVLQSSGLYSLSSVVTVPSSSLGTKTYTCNVDHKPS |
| | dashed | NTKVDKRVESKYGPPCPPCPAPEFLGGPSVFLFPPKPKDTLMISRTPEVTCVVVDVSQEDPEVQENWYV |
| | underline | DGVEVHNAKTKPREEQFNSTYRVVSVLTVLHQDWLNGKEYKCKVSNKGLPSSIEKTISKAKGQPREPQV |
| | hIL-2 in | YTLPPSQEEMTKNQVSLTCLVKGFYPSDIAVEWESNGQPENNYKTTPPVLDSDGSFFLYSRLTVDKSRW |
| | italics | QEGNVFSCSVMHEALHNHYTQKSLSLSLGK 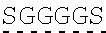 APTSSSTKKTQLQLEHLLLDL |
| | | QMILNGINNYKNPKLTRMLTFKFYMPKKATELKHLQCLEEELKPLEEVLNLAQSKNFHLR |
| | | PRDLISNINVIVLELKGSETTFMCEYADETATIVEFLNRWITFCQSIISTLT |
| 482 | 1H3-hIgG4 HC | EVQLVESGGGLVQPGRSLKLSCAVSGFTFSDYAMAWVRQAPKKGLEWVATISYDGSRTYYRDSVKGRFT |
| | | ISRDNAKITLYLQMDSLRSEDTATYYCARHGSGYFDYWGQGVMVTVSSASTKGPSVFPLAPCSRSTSES |
| | | TAALGCLVKDYFPEPVTVSWNSGALTSGVHTFPAVLOSSGLYSLSSVVTVPSSSLGTKTYTCNVDHKPS |
| | | NTKVDKRVESKYGPPCPPCPAPEFLGGPSVFLFPPKPKDTLMISRTPEVTCVVVDVSQEDPEVQFNWYV |
| | | DGVEVHNAKTKPREEQFNSTYRVVSVLTVLHQDWLNGKEYKCKVSNKGLPSSIEKTISKAKGQPREPQV |
| | | YTLPPSQEEMTKNQVSLTCLVKGFYPSDIAVEWESNGQPENNYKTTPPVLDSDGSFFLYSRLTVDKSRW |
| | | QEGNVFSCSVMHEALHNHYTQKSLSLSLG |
| 483 | 1H3-hKappa-df- | DTVLTQSPALAVSPGERVTISCRASESVRTGVHWYQQKPGQQPKLLIYGASNLESGVPARFSGSGSGTD |
| | hIL-2 (WT) LC | FTLTIDPVEADDTATYFCQQSWNDPFTFGSGTKLEIKRTVAAPSVFIFPPSDEQLKSGTASVVCLLNNE |
| | hIL-2 in | YPREAKVQWKVDNALQSGNSQESVTEQDSKDSTYSLSSTLTLSKADYEKHKVYACEVTHQGLSSPVTKS |
| | italics | FNRGECAPTSSSTKKTQLQLEHLLLDLOMILNGINNYKNPKLTRMLTFKFYMPKKATELKHLQCLEEEL |
| | | KPLEEVLNLAQSKNFHLRPRDLISNINVIVLELKGSETTEMCEYADETATIVEFLNRWITFCQSIISTL |
| | | T |
| 484 | 1H3-hKappa-L6- | DTVLTQSPALAVSPGERVTISCRASESVRTGVHWYQQKPGQQPKLLIYGASNLESGVPARFSGSGSGTD |
| | hIL-2 (WT) LC | FTLTIDPVEADDTATYFCQQSWNDPFTFGSGTKLEIKRTVAAPSVFIFPPSDEQLKSGTASVVCLLNNE |
| | Linker in | YPREAKVQWKVDNALQSGNSQESVTEQDSKDSTYSLSSTLTLSKADYEKHKVYACEVTHQGLSSPVTKS |
| | dashed | FNRGEC 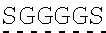 APTSSSTKKTQLQLEHLLLDLQMILNGINNYKNP |
| | underline | KLTRMLTEKFYMPKKATELKHLQCLEEELKPLEEVLNLAQSKNFHLRPR |
| | hIL-2 in | DLISNINVIVLELKGSETTEMCEYADETATIVEFLNRWITECSIIQSTLT |
| | italics | |
| 485 | 1H3-hIgG4-L6- | EVQLVESGGGLVQPGRSLKLSCAVSGFTFSDYAMAWVRQAPKKGLEWVATISYDGSRTYYRDSVKGRFT |
| | hIL-2 (D20Y) | ISRDNAKITLYLQMDSLRSEDTATYYCARHGSGYFDYWGQGVMVTVSSASTKGPSVFPLAPCSRSTSES |
| | HC | TAALGCLVKDYFPEPVTVSWNSGALTSGVHTFPAVLQSSGLYSLSSVVTVPSSSLGTKTYTCNVDHKPS |
| | Linker in | NTKVDKRVESKYGPPCPPCPAPEFLGGPSVFLFPPKPKDTLMISRTPEVTCVVVDVSQEDPEVQFNWYV |
| | dashed | DGVEVHNAKTKPREEQFNSTYRVVSVLTVLHQDWLNGKEYKCKVSNKGLPSSIEKTISKAKGQPREPQV |
| | underline | YTLPPSQEEMTKNQVSLTCLVKGFYPSDIAVEWESNGQPENNYKTTPPVLDSDGSFFLYSRLTVDKSRW |
| | hIL-2 in | QEGNVFSCSVMHEALHNHYTQKSLSLSLGK 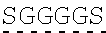 APTSSSTKKTQLQLEHLLLYL |
| | italics | QMILNGINNYKNPKLTRMLTFKFYMPKKATELKHLQCLEEELKPLEEVLNLAQ |
| | | SKNFHLRPRDLISNINVIVLELKGSETTFMCEYADETATIVEFLNRWITFCQSIISTLT |
| 486 | 1H3-hIgG4-df- | EVQLVESGGGLVQPGRSLKLSCAVSGFTFSDYAMAWVRQAPKKGLEWVATISYDGSRTYYRDSVKGRFT |
| | hIL-2 (D20Y) | ISRDNAKITLYLQMDSLRSEDTATYYCARHGSGYFDYWGQGVMVTVSSASTKGPSVFPLAPCSRSTSES |
| | HC | TAALGCLVKDYFPEPVTVSWNSGALTSGVHTFPAVLQSSGLYSLSSVVTVPSSSLGTKTYTCNVDHKPS |
| | hIL-2 in | NTKVDKRVESKYGPPCPPCPAPEFLGGPSVFLFPPKPKDTLMISRTPEVTCVVVDVSQEDPEVQFNWYV |
| | italics | DGVEVHNAKTKPREEQFNSTYRVVSVLTVLHQDWLNGKEYKCKVSNKGLPSSIEKTISKAKGQPREPQV |
| | | YTLPPSQEEMTKNQVSLTCLVKGFYPSDIAVEWESNGQPENNYKTTPPVLDSDGSFFLYSRLTVDKSRW |
| | | QEGNVFSCSVMHEALHNHYTQKSLSLSLGKAPTSSSTKKTQLQLEHLLLYLOMILNGINNYKNPKLTRM |
| | | LTFKFYMPKKATELKHLQCLEEELKPLEEVLNLAQSKNFHLRPRDLISNINVIVLELKGSETTEMCEYA |
| | | DETATIVEFLNRWITFCQSIISTLT |
| 487 | 1H3-hIgG1-df- | EVQLVESGGGLVQPGRSLKLSCAVSGFTESDYAMAWVRQAPKKGLEWVATISYDGSRTYYRDSVKGRET |
| | hIL-2 (D20Y) | ISRDNAKITLYLQMDSLRSEDTATYYCARHGSGYFDYWGQGVMVTVSSASTKGPSVFPLAPSSKSTSGG |
| | HC | TAALGCLVKDYFPEPVTVSWNSGALTSGVHTFPAVLQSSGLYSLSSVVTVPSSSLGTQTYICNVNHKPS |
| | hIL-2 in | NTKVDKKVEPKSCDKTHTCPPCPAPELLGGPSVFLFPPKPKDTLMISRTPEVTCVVVDVSHEDPEVKEN |
| | italics | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKAPTSSSTKKTQLQLEHLLLYLQMILNGINNYKNPKL |
| | | TRMLTFKFYMPKKATELKHLQCLEEELKPLEEVLNLAQSKNFHLRPRDLISNINVIVLELKGSETTEMC |
| | | EYADETATIVEFLNRWITFCQSIISTLT |
| 488 | 1H3-hIgG4-L6- | EVOLVESGGGLVQPGRSLKLSCAVSGFTFSDYAMAWVRQAPKKGLEWVATISYDGSRTYYRDSVKGRFT |
| | hIL-2 | ISRDNAKITLYLOMDSLRSEDTATYYCARHGSGYFDYWGQGVMVTVSSASTKGPSVFPLAPCSRSTSES |
| | (D20A/R38P) HC | TAALGCLVKDYFPEPVTVSWNSGALTSGVHTFPAVLOSSGLYSLSSVVTVPSSSLGTKTYTCNVDHKPS |
| | Linker in | NTKVDKRVESKYGPPCPPCPAPEFLGGPSVFLFPPKPKDTLMISRTPEVTCVVVDVSQEDPEVQENWYV |
| | dashed | DGVEVHNAKTKPREEQFNSTYRVVSVLTVLHQDWLNGKEYKCKVSNKGLPSSIEKTISKAKGQPREPQV |
| | underline | YTLPPSQEEMTKNQVSLTCLVKGFYPSDIAVEWESNGQPENNYKTTPPVLDSDGSFFLYSRLTVDKSRW |
| | hIL-2 in | QEGNVFSCSVMHEALHNHYTQKSLSLSLGK 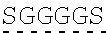 APTSSSTKKT |
| | italics | QLQLEHLLLALQMILNGINNYKNPKLTPMLTFKFYMPKKATELKHLOCLEEELKPLEEVLNLAQ |
| | | SKNFHLRPRDLISNINVIVLELKGSETTFMCEYADETATIVEFLNRWITFCQSIISTLT |
| 489 | 1H3-hIgG4-L6- | EVQLVESGGGLVQPGRSLKLSCAVSGFTFSDYAMAWVRQAPKKGLEWVATISYDGSRTYYRDSVKGRFT |
| | hIL-2 | ISRDNAKITLYLQMDSLRSEDTATYYCARHGSGYFDYWGQGVMVTVSSASTKGPSVFPLAPCSRSTSES |
| | (D20A/R38S) HC | TAALGCLVKDYFPEPVTVSWNSGALTSGVHTFPAVLQSSGLYSLSSVVTVPSSSLGTKTYTCNVDHKPS |
| | Linker in | NTKVDKRVESKYGPPCPPCPAPEFLGGPSVFLFPPKPKDTLMISRTPEVTCVVVDVSQEDPEVQENWYV |
| | dashed | DGVEVHNAKTKPREEQFNSTYRVVSVLTVLHQDWLNGKEYKCKVSNKGLPSSIEKTISKAKGQPREPQV |
| | underline | YTLPPSQEEMTKNQVSLTCLVKGFYPSDIAVEWESNGQPENNYKTTPPVLDSDGSFFLYSRLTVDKSRW |
| | hIL-2 in | QEGNVFSCSVMHEALHNHYTQKSLSLSLGK 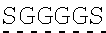 APTSSSTKKTQLQL |
| | italics | EHLLLALOMILNGINNYKNPKLTSMLTFKFYMPKKATELKHLQCLEEELKPLEEVLNL |
| | | AQSKNFHLRPRDLISNINVIVLELKGSETTFMCEYADETATIVEFLNRWITFCQSIISTLT |
| 490 | 1H3-hIgG4-L6- | EVQLVESGGGLVQPGRSLKLSCAVSGFTFSDYAMAWVRQAPKKGLEWVATISYDGSRTYYRDSVKGRFT |
| | hIL-2 | ISRDNAKITLYLQMDSLRSEDTATYYCARHGSGYFDYWGQGVMVTVSSASTKGPSVEPLAPCSRSTSES |
| | (D20A/R38D) HC | TAALGCLVKDYFPEPVTVSWNSGALTSGVHTFPAVLQSSGLYSLSSVVTVPSSSLGTKTYTCNVDHKPS |
| | Linker in | NTKVDKRVESKYGPPCPPCPAPEFLGGPSVELFPPKPKDTLMISRTPEVTCVVVDVSQEDPEVQENWYV |
| | dashed | DGVEVHNAKTKPREEQFNSTYRVVSVLTVLHQDWLNGKEYKCKVSNKGLPSSIEKTISKAKGQPREPQV |
| | underline | YTLPPSQEEMTKNQVSLTCLVKGFYPSDIAVEWESNGQPENNYKTTPPVLDSDGSFFLYSRLTVDKSRW |
| | hIL-2 in | QEGNVFSCSVMHEALHNHYTQKSLSLSLGK 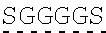 APTSSSTKKTQLQL |
| | italics | EHLLLALQMILNGINNYKNPKLTDMLTFKFYMPKKATELKHLQCLEEELKPLEEVLNLAQSKNF |
| | | HLRPRDLISNINVIVLELKGSETTFMCEYADETATIVEFLNRWITFCQSIISTLT |
| 491 | 1H3-hIgG4-L6- | EVQLVESGGGLVQPGRSLKLSCAVSGFTFSDYAMAWVRQAPKKGLEWVATISYDGSRTYYRDSVKGRFT |
| | hIL-2 | ISRDNAKITLYLQMDSLRSEDTATYYCARHGSGYFDYWGQGVMVTVSSASTKGPSVFPLAPCSRSTSES |
| | (D20A/R38Q/E95 | TAALGCLVKDYFPEPVTVSWNSGALTSGVHTFPAVLQSSGLYSLSSVVTVPSSSLGTKTYTCNVDHKPS |
| | A) HC | NTKVDKRVESKYGPPCPPCPAPEFLGGPSVFLFPPKPKDTLMISRTPEVTCVVVDVSQEDPEVQENWYV |
| | Linker in | DGVEVHNAKTKPREEQFNSTYRVVSVLTVLHQDWLNGKEYKCKVSNKGLPSSIEKTISKAKGQPREPQV |
| | dashed | YTLPPSQEEMTKNQVSLTCLVKGFYPSDIAVEWESNGQPENNYKTTPPVLDSDGSFFLYSRLTVDKSRW |
| | underline | QEGNVFSCSVMHEALHNHYTQKSLSLSLGK 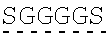 APTSSSTKKTQLQLEHL |
| | hIL-2 in | LLALQMILNGINNYKNPKLTQMLTFKFYMPKKATELKHLQCLEEELKPLEEVLNL |
| | italics | AQSKNFHLRPRDLISNINVIVLALKGSETTFMCEYADETATIVEFLNRWITFCQSIISTLT |
| 492 | 1H3-hIgG4-L6- | EVQLVESGGGLVQPGRSLKLSCAVSGFTFSDYAMAWVRQAPKKGLEWVATISYDGSRTYYRDSVKGRET |
| | hIL-2 | ISRDNAKITLYLQMDSLRSEDTATYYCARHGSGYFDYWGQGVMVTVSSASTKGPSVFPLAPCSRSTSES |
| | (D20A/F42H/E95 | TAALGCLVKDYFPEPVTVSWNSGALTSGVHTFPAVLQSSGLYSLSSVVTVPSSSLGTKTYTCNVDHKPS |
| | A) HC | NTKVDKRVESKYGPPCPPCPAPEFLGGPSVFLFPPKPKDTLMISRTPEVTCVVVDVSQEDPEVQFNWYV |
| | Linker in | DGVEVHNAKTKPREEQFNSTYRVVSVLTVLHQDWLNGKEYKCKVSNKGLPSSIEKTISKAKGQPREPQV |
| | dashed | YTLPPSQEEMTKNQVSLTCLVKGFYPSDIAVEWESNGQPENNYKTTPPVLDSDGSFFLYSRLTVDKSRW |
| | underline | QEGNVFSCSVMHEALHNHYTQKSLSLSLGK 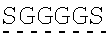 APTSSSTKKTQLQL |
| | hIL-2 in | EHLLLALQMILNGINNYKNPKLTRMLTHKFYMPKKATELKHLQCLEEELKPLEEVLNLAQS |
| | italics | KNFHLRPRDLISNINVIVLALKGSETTFMCEYADETATIVEFLNRWITFCQSIISTLT |
| 493 | 1H3-hIgG4-L6- | EVQLVESGGGLVQPGRSLKLSCAVSGFTFSDYAMAWVRQAPKKGLEWVATISYDGSRTYYRDSVKGRET |
| | hIL-2 | ISRDNAKITLYLQMDSLRSEDTATYYCARHGSGYFDYWGQGVMVTVSSASTKGPSVFPLAPCSRSTSES |
| | (R38D/I92D) HC | TAALGCLVKDYFPEPVTVSWNSGALTSGVHTFPAVLQSSGLYSLSSVVTVPSSSLGTKTYTCNVDHKPS |
| | Linker in | NTKVDKRVESKYGPPCPPCPAPEFLGGPSVFLFPPKPKDTLMISRTPEVTCVVVDVSQEDPEVQENWYV |
| | dashed | DGVEVHNAKTKPREEQFNSTYRVVSVLTVLHQDWLNGKEYKCKVSNKGLPSSIEKTISKAKGQPREPQV |
| | underline | YTLPPSQEEMTKNQVSLTCLVKGFYPSDIAVEWESNGQPENNYKTTPPVLDSDGSFFLYSRLTVDKSRW |
| | hIL-2 in | QEGNVFSCSVMHEALHNHYTQKSLSLSLGK 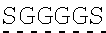 APTSSSTKKTQLQLE |
| | italics | HLLLDLQMILNGINNYKNPKLTDMLTFKFYMPKKATELKHLQCLEEELKPLEEVLNLAQSKNF |
| | | HLRPRDLISNINVDVLELKGSETTFMCEYADETATIVEFLNRWITFCQSIISTLT |
| 494 | 1H3-hIgG4-L6- | EVQLVESGGGLVQPGRSLKLSCAVSGFTFSDYAMAWVRQAPKKGLEWVATISYDGSRTYYRDSVKGRET |
| | hIL-2 | ISRDNAKITLYLQMDSLRSEDTATYYCARHGSGYFDYWGQGVMVTVSSASTKGPSVFPLAPCSRSTSES |
| | (R38E/I92D) HC | TAALGCLVKDYFPEPVTVSWNSGALTSGVHTFPAVLQSSGLYSLSSVVTVPSSSLGTKTYTCNVDHKPS |
| | Linker in | NTKVDKRVESKYGPPCPPCPAPEFLGGPSVFLFPPKPKDTLMISRTPEVTCVVVDVSQEDPEVQENWYV |
| | dashed | DGVEVHNAKTKPREEQFNSTYRVVSVLTVLHQDWLNGKEYKCKVSNKGLPSSIEKTISKAKGQPREPQV |
| | underline | YTLPPSQEEMTKNQVSLTCLVKGFYPSDIAVEWESNGQPENNYKTTPPVLDSDGSFFLYSRLTVDKSRW |
| | hIL-2 in | QEGNVFSCSVMHEALHNHYTQKSLSLSLGK 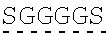 APTSSSTKKTQLQLEHL |
| | italics | LLDLQMILNGINNYKNPKLTEMLTFKFYMPKKATELKHLQCLEEELKPLEEVLNLAQSKNFHLRPR |
| | | DLISNINVDVLELKGSETTFMCEYADETATIVEFLNRWITFCQSIISTLT |
| 495 | 1H3-hIgG4-L6- | EVQLVESGGGLVQPGRSLKLSCAVSGFTFSDYAMAWVRQAPKKGLEWVATISYDGSRTYYRDSVKGRFT |
| | hIL-2 | ISRDNAKITLYLQMDSLRSEDTATYYCARHGSGYFDYWGQGVMVTVSSASTKGPSVEPLAPCSRSTSES |
| | (F42H/I92D) HC | TAALGCLVKDYFPEPVTVSWNSGALTSGVHTFPAVLOSSGLYSLSSVVTVPSSSLGTKTYTCNVDHKPS |
| | Linker in | NTKVDKRVESKYGPPCPPCPAPEFLGGPSVFLFPPKPKDTLMISRTPEVTCVVVDVSQEDPEVQFNWYV |
| | dashed | DGVEVHNAKTKPREEQFNSTYRVVSVLTVLHQDWLNGKEYKCKVSNKGLPSSIEKTISKAKGQPREPQV |
| | underline | YTLPPSQEEMTKNQVSLTCLVKGFYPSDIAVEWESNGQPENNYKTTPPVLDSDGSFFLYSRLTVDKSRW |
| | hIL-2 in | QEGNVFSCSVMHEALHNHYTQKSLSLSLGK 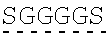 APTSSSTKKTQLQLEHLL |
| | italics | NLDLQMILNGINNYKPKLTRMLTHKFYMPKKATELKHLQCLEEELKPLEEVLNLAQSKNF |
| | | HLRPRDLISNINVDVLELKGSETTFMCEYADETATIVEFLNRWITFCQSIISTLT |
| 496 | 1H3-hIgG4-L6- | EVQLVESGGGLVQPGRSLKLSCAVSGFTFSDYAMAWVRQAPKKGLEWVATISYDGSRTYYRDSVKGRET |
| | hIL-2 | ISRDNAKITLYLOMDSLRSEDTATYYCARHGSGYFDYWGQGVMVTVSSASTKGPSVFPLAPCSRSTSES |
| | (D20A/R38E) HC | TAALGCLVKDYFPEPVTVSWNSGALTSGVHTFPAVLQSSGLYSLSSVVTVPSSSLGTKTYTCNVDHKPS |
| | Linker in | NTKVDKRVESKYGPPCPPCPAPEFLGGPSVFLFPPKPKDTLMISRTPEVTCVVVDVSQEDPEVQFNWYV |
| | dashed | DGVEVHNAKTKPREEQFNSTYRVVSVLTVLHQDWLNGKEYKCKVSNKGLPSSIEKTISKAKGQPREPQV |
| | underline | YTLPPSQEEMTKNQVSLTCLVKGFYPSDIAVEWESNGQPENNYKTTPPVLDSDGSFFLYSRLTVDKSRW |
| | hIL-2 in | QEGNVFSCSVMHEALHNHYTQKSLSLSLGK 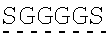 APTSSSTKKTQLQLEHLLLA |
| | italics | LQMILNGINNYKNPKLTEMLTFKFYMPKKATELKHLQCLEEELKPLEEVLNLAQSKN |
| | | FHLRPRDLISNINVIVLELKGSETTFMCEYADETATIVEFLNRWITFCQSIISTLT |
| 497 | 1H3-hIgG4-L6- | EVQLVESGGGLVQPGRSLKLSCAVSGFTESDYAMAWVRQAPKKGLEWVATISYDGSRTYYRDSVKGRET |
| | hIL-2 | ISRDNAKITLYLQMDSLRSEDTATYYCARHGSGYFDYWGQGVMVTVSSASTKGPSVEPLAPCSRSTSES |
| | (T3A/D20A/R38E) | TAALGCLVKDYFPEPVTVSWNSGALTSGVHTFPAVLQSSGLYSLSSVVTVPSSSLGTKTYTCNVDHKPS |
| | HC | NTKVDKRVESKYGPPCPPCPAPEFLGGPSVFLFPPKPKDTLMISRTPEVTCVVVDVSQEDPEVQENWYV |
| | Linker in | DGVEVHNAKTKPREEQFNSTYRVVSVLTVLHQDWLNGKEYKCKVSNKGLPSSIEKTISKAKGQPREPQV |
| | dashed | YTLPPSQEEMTKNQVSLTCLVKGFYPSDIAVEWESNGQPENNYKTTPPVLDSDGSFFLYSRLTVDKSRW |
| | underline | QEGNVFSCSVMHEALHNHYTQKSLSLSLGK 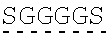 APASSSTKKTQLQLEH |
| | hIL-2 in | LLLALQMILNGINNYKNPKLTEMLTFKFYMPKKATELKHLQCLEEELKPLEEVLNLAQSKNF |
| | italics | HLRPRDLISNINVIVLELKGSETTFMCEYADETATIVEFLNRWITFCQSIISTLT |
| 498 | 1H3-hIgG4-L6- | EVQLVESGGGLVQPGRSLKLSCAVSGFTFSDYAMAWVRQAPKKGLEWVATISYDGSRTYYRDSVKGRFT |
| | hIL-2 | ISRDNAKITLYLQMDSLRSEDTATYYCARHGSGYFDYWGQGVMVTVSSASTKGPSVFPLAPCSRSTSES |
| | (D20A/R38E/C12 | TAALGCLVKDYFPEPVTVSWNSGALTSGVHTFPAVLOSSGLYSLSSVVTVPSSSLGTKTYTCNVDHKPS |
| | 5A) HC | NTKVDKRVESKYGPPCPPCPAPEFLGGPSVFLFPPKPKDTLMISRTPEVTCVVVDVSQEDPEVQENWYV |
| | Linker in | DGVEVHNAKTKPREEQFNSTYRVVSVLTVLHQDWLNGKEYKCKVSNKGLPSSIEKTISKAKGQPREPQV |
| | dashed | YTLPPSQEEMTKNQVSLTCLVKGFYPSDIAVEWESNGQPENNYKTTPPVLDSDGSFFLYSRLTVDKSRW |
| | underline | QEGNVFSCSVMHEALHNHYTQKSLSLSLGK 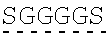 APTSSSTKKTQLQLEHLLLA |
| | hIL-2 in | LQMILNGINNYKNPKLTEMLTFKFYMPKKATELKHLQCLEEELKPLEEVLNLAQSKNFHL |
| | italics | RPRDLISNINVIVLELKGSETTFMCEYADETATIVEFLNRWITFAQSIISTLT |
| 499 | 1H3-hIgG4-L6- | EVQLVESGGGLVQPGRSLKLSCAVSGFTFSDYAMAWVRQAPKKGLEWVATISYDGSRTYYRDSVKGRFT |
| | hIL-2 | ISRDNAKITLYLQMDSLRSEDTATYYCARHGSGYFDYWGQGVMVTVSSASTKGPSVFPLAPCSRSTSES |
| | (T3A/D20A/R38E | TAALGCLVKDYFPEPVTVSWNSGALTSGVHTFPAVLQSSGLYSLSSVVTVPSSSLGTKTYTCNVDHKPS |
| | /C125A) HC | NTKVDKRVESKYGPPCPPCPAPEFLGGPSVELFPPKPKDTLMISRTPEVTCVVVDVSQEDPEVQENWYV |
| | Linker in | DGVEVHNAKTKPREEQFNSTYRVVSVLTVLHQDWLNGKEYKCKVSNKGLPSSIEKTISKAKGQPREPQV |
| | dashed | YTLPPSQEEMTKNQVSLTCLVKGFYPSDIAVEWESNGQPENNYKTTPPVLDSDGSFFLYSRLTVDKSRW |
| | underline | QEGNVFSCSVMHEALHNHYTQKSLSLSLGK 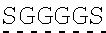 APASSSTKKTQLQLEHLL |
| | hIL-2 in | LALQMILNGINNYKNPKLTEMLTFKFYMPKKATELKHLQCLEEELKPLEEVLNLAQSKNF |
| | italics | HLRPRDLISNINVIVLELKGSETTFMCEYADETATIVEFLNRWITFAQSIISTLT |
| 500 | 1H3-hIgG1-L6- | EVQLVESGGGLVQPGRSLKLSCAVSGFTFSDYAMAWVRQAPKKGLEWVATISYDGSRTYYRDSVKGRFT |
| | hIL-2 | ISRDNAKITLYLQMDSLRSEDTATYYCARHGSGYFDYWGQGVMVTVSSASTKGPSVFPLAPSSKSTSGG |
| | (D20A/R38E) HC | TAALGCLVKDYFPEPVTVSWNSGALTSGVHTFPAVLQSSGLYSLSSVVTVPSSSLGTQTYICNVNHKPS |
| | Linker in | NTKVDKKVEPKSCDKTHTCPPCPAPELLGGPSVFLFPPKPKDTLMISRTPEVTCVVVDVSHEDPEVKEN |
| | dashed | WYVDGVEVHNAKTKPREEQYNSTYRVVSVLTVLHQDWLNGKEYKCKVSNKALPAPIEKTISKAKGQPRE |
| | underline | PQVYTLPPSRDELTKNQVSLTCLVKGFYPSDIAVEWESNGQPENNYKTTPPVLDSDGSFFLYSKLTVDK |
| | hIL-2 in | SRWQQGNVFSCSVMHEALHNHYTQKSLSLSPGK 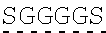 APTSSSTKKTQ |
| | italics | LQLEHLLLALQMILNGINNYKNPKLTEMLTFKFYMPKKATELKHLQCLEEELKPLEEVLNLAQSK |
| | | NFHLRPRDLISNINVIVLELKGSETTEMCEYADETATIVEFLNRWITFCQSIISTLT |
| 501 | 1H3-hIgG1-L6- | EVOLVESGGGLVQPGRSLKLSCAVSGFTFSDYAMAWVRQAPKKGLEWVATISYDGSRTYYRDSVKGRET |
| | hIL-2 | ISRDNAKITLYLQMDSLRSEDTATYYCARHGSGYFDYWGQGVMVTVSSASTKGPSVFPLAPSSKSTSGG |
| | (T3A/D20A/R38E) | TAALGCLVKDYFPEPVTVSWNSGALTSGVHTFPAVLQSSGLYSLSSVVTVPSSSLGTQTYICNVNHKPS |
| | HC | NTKVDKKVEPKSCDKTHTCPPCPAPELLGGPSVELFPPKPKDTLMISRTPEVTCVVVDVSHEDPEVKEN |
| | Linker in | WYVDGVEVHNAKTKPREEQYNSTYRVVSVLTVLHQDWLNGKEYKCKVSNKALPAPIEKTISKAKGQPRE |
| | dashed | PQVYTLPPSRDELTKNQVSLTCLVKGFYPSDIAVEWESNGQPENNYKTTPPVLDSDGSFFLYSKLTVDK |
| | underline | SRWQQGNVFSCSVMHEALHNHYTQKSLSLSPGK 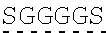 APASSSTKKTQLQL |
| | hIL-2 in | EHLLLALQMILNGINNYKNPKLTEMLTFKFYMPKKATELKHLQCLEEELKPLEEVLNL |
| | italics | AQSKNFHLRPRDLISNINVIVLELKGSETTFMCEYADETATIVEFLNRWITFCQSIISTLT |
| 502 | 1H3-hIgG1-L6- | EVQLVESGGGLVQPGRSLKLSCAVSGFTFSDYAMAWVRQAPKKGLEWVATISYDGSRTYYRDSVKGRFT |
| | hIL-2 | ISRDNAKITLYLQMDSLRSEDTATYYCARHGSGYFDYWGQGVMVTVSSASTKGPSVFPLAPSSKSTSGG |
| | (D20A/R38E/C12 | TAALGCLVKDYFPEPVTVSWNSGALTSGVHTFPAVLQSSGLYSLSSVVTVPSSSLGTQTYICNVNHKPS |
| | 5A) HC | NTKVDKKVEPKSCDKTHTCPPCPAPELLGGPSVFLFPPKPKDTLMISRTPEVTCVVVDVSHEDPEVKEN |
| | Linker in | WYVDGVEVHNAKTKPREEQYNSTYRVVSVLTVLHQDWLNGKEYKCKVSNKALPAPIEKTISKAKGQPRE |
| | dashed | PQVYTLPPSRDELTKNQVSLTCLVKGFYPSDIAVEWESNGQPENNYKTTPPVLDSDGSFFLYSKLTVDK |
| | underline | SRWQQGNVFSCSVMHEALHNHYTQKSLSLSPGK 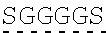 APTSSSTKKTQLQ |
| | hIL-2 in | LEHLLLALQMILNGINNYKNPKLTEMLTFKFYMPKKATELKHLQCLEEELKPLEEVLNL |
| | italics | AQSKNFHLRPRDLISNINVIVLELKGSETTFMCEYADETATIVEFLNRWITFAQSIISTLT |
| 503 | 1H3-hKappa-df- | DTVLTQSPALAVSPGERVTISCRASESVRTGVHWYQQKPGQQPKLLIYGASNLESGVPARFSGSGSGTD |
| | hIL-2 | FTLTIDPVEADDTATYFCQQSWNDPFTFGSGTKLEIKRTVAAPSVFIFPPSDEQLKSGTASVVCLLNNE |
| | (D20A/R38E) LC | YPREAKVQWKVDNALQSGNSQESVTEQDSKDSTYSLSSTLTLSKADYEKHKVYACEVTHQGLSSPVTKS |
| | hIL-2 in | FNRGECAPTSSSTKKTQLQLEHLLLALQMILNGINNYKNPKLTEMLTFKFYMPKKATELKHLQCLEEEL |
| | italics | KPLEEVLNLAQSKNFHLRPRDLISNINVIVLELKGSETTEMCEYADETATIVEFLNRWITFCQSIISTL |
| | | T |
| 504 | 1H3-hKappa-L6- | DTVLTQSPALAVSPGERVTISCRASESVRTGVHWYQQKPGQQPKLLIYGASNLESGVPARFSGSGSGTD |
| | hIL-2 | FTLTIDPVEADDTATYFCQQSWNDPFTFGSGTKLEIKRTVAAPSVFIFPPSDEQLKSGTASVVCLLNNE |
| | (D20A/R38E) LC | YPREAKVQWKVDNALQSGNSQESVTEQDSKDSTYSLSSTLTLSKADYEKHKVYACEVTHQGLSSPVTKS |
| | Linker in | FNRGEC 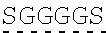 APTSSSTKKTQLQLEHLLLALQMILNGINNYKNPKLTEMLT |
| | dashed | FKFYMPKKATELKHLQCLEEELKPLEEVLNLAQSKNFHLRPRDLISNINVIVLELKGSE |
| | underline | TTEMCEYADETATIVEFLNRWITFCQSIISTLT |
| | hIL-2 in | |
| | italics | |
| 505 | OMC476pB11- | DVQLVESGGGVVRPGESLRLSCTASGFTFSISAMSWVRQAPGKGLEWVSAISGTAYSTYYADSVRGRFT |
| | hIgG4-df-hIL-2 | ISRDNSKNTLYLQMNSLRAEDTAVYYCAKDNFFDYWGLGTLVTVSSASTKGPSVFPLAPCSRSTSESTA |
| | (D20A/R38E) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLALQMILNGINNYKNPKLTEMLT |
| | | FKFYMPKKATELKHLOCLEEELKPLEEVLNLAQSKNFHLRPRDLISNINVIVLELKGSETTEMCEYADE |
| | | TATIVEFLNRWITFCQSIISTLT |
| 506 | OMC476pE4- | DVQLVESGGGVVRPGESLRLSCAASGFTFSTDAMGWVRQAPGEGLEWVSLISGSGYSTYYADSVKGRFT |
| | hIgG4-df-hIL-2 | ISRDNSKNTLYLQMNSLTAEDTAVYYCAKNSLAFFDYWGLGTLVTVSSASTKGPSVFPLAPCSRSTSES |
| | (D20A/R38E) HC | TAALGCLVKDYFPEPVTVSWNSGALTSGVHTFPAVLQSSGLYSLSSVVTVPSSSLGTKTYTCNVDHKPS |
| | hIL-2 in | NTKVDKRVESKYGPPCPPCPAPEFLGGPSVFLFPPKPKDTLMISRTPEVTCVVVDVSQEDPEVQENWYV |
| | italics | DGVEVHNAKTKPREEQFNSTYRVVSVLTVLHQDWLNGKEYKCKVSNKGLPSSIEKTISKAKGQPREPQV |
| | | YTLPPSQEEMTKNQVSLTCLVKGFYPSDIAVEWESNGQPENNYKTTPPVLDSDGSFFLYSRLTVDKSRW |
| | | QEGNVFSCSVMHEALHNHYTQKSLSLSLGKAPTSSSTKKTQLQLEHLLLALQMILNGINNYKNPKLTEM |
| | | LTFKFYMPKKATELKHLQCLEEELKPLEEVLNLAQSKNFHLRPRDLISNINVIVLELKGSETTEMCEYA |
| | | DETATIVEFLNRWITFCQSIISTLT |
| 507 | OMC476pG10- | DVQLVESGGGVVRPGGSLRLSCAASGFTFSIYAMSWVRQAPGEGLEWVSHISASGGSTYYADSVKGRFA |
| | hIgG4-df-hIL-2 | ISRDNSKNTLYLQMNSLRAEDTAVYYCTTNLGSDYWGLGTLVTVSSASTKGPSVFPLAPCSRSTSESTA |
| | (D20A/R38E) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLALQMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFCQSIISTLT |
| 508 | OMC476pH10- | DVQLVESGGGVVRPGGSLRLSCAASGFTFSIYAVSWVRQAPGEGLEWVSHISASGGSTYYADSVKGRFA |
| | hIgG4-df-hIL-2 | ISRDNSKNTLYLQMNSLRAEDTAVYYCTTNLGSDYWGLGTLVTVSSASTKGPSVFPLAPCSRSTSESTA |
| | (D20A/R38E) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLALQMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFCQSIISTLT |
| 509 | A2-hIgG4-df- | DVQLVESGGGLVQPGGSLRLSCAASGFTFDISAMSWVRQAPGKGLEWVSTISGSAYSTYYADSVKGRET |
| | hIL-2 | ISRDNSKSTLYLQMNSLRAEDTAVYYCAREIFSDYWGLGTLVTVSSASTKGPSVFPLAPCSRSTSESTA |
| | (D20A/F42A) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLALQMILNGINNYKNPKLTRMLT |
| | | AKFYMPKKATELKHLQCLEEELKPLEEVLNLAQSKNFHLRPRDLISNINVIVLELKGSETTEMCEYADE |
| | | TATIVEFLNRWITFCQSIISTLT |
| 510 | A2-hIgG4-df- | DVQLVESGGGLVQPGGSLRLSCAASGFTFDISAMSWVRQAPGKGLEWVSTISGSAYSTYYADSVKGRFT |
| | hIL-2 | ISRDNSKSTLYLQMNSLRAEDTAVYYCAREIFSDYWGLGTLVTVSSASTKGPSVFPLAPCSRSTSESTA |
| | (D20A/F42S) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVELFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLALQMILNGINNYKNPKLTRMLT |
| | | SKFYMPKKATELKHLQCLEEELKPLEEVLNLAQSKNFHLRPRDLISNINVIVLELKGSETTEMCEYADE |
| | | TATIVEFLNRWITFCQSIISTLT |
| 511 | A2-hIgG4-df- | DVQLVESGGGLVQPGGSLRLSCAASGFTFDISAMSWVRQAPGKGLEWVSTISGSAYSTYYADSVKGRET |
| | hIL-2 | ISRDNSKSTLYLQMNSLRAEDTAVYYCAREIFSDYWGLGTLVTVSSASTKGPSVFPLAPCSRSTSESTA |
| | (D20S/R38E) HC | ALGCLVKDYFPEPVTVSWNSGALTSGVHTFPAVLOSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLSLOMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFCQSIISTLT |
| 512 | A2-hIgG4-df- | DVQLVESGGGLVQPGGSLRLSCAASGFTFDISAMSWVRQAPGKGLEWVSTISGSAYSTYYADSVKGRFT |
| | hIL-2 | ISRDNSKSTLYLQMNSLRAEDTAVYYCAREIFSDYWGLGTLVTVSSASTKGPSVFPLAPCSRSTSESTA |
| | (F42A/N88R) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQENWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLDLQMILNGINNYKNPKLTRMLT |
| | | AKFYMPKKATELKHLQCLEEELKPLEEVLNLAQSKNFHLRPRDLISRINVIVLELKGSETTEMCEYADE |
| | | TATIVEFLNRWITFCQSIISTLT |
| 513 | A2-hIgG4-df- | DVQLVESGGGLVQPGGSLRLSCAASGFTFDISAMSWVRQAPGKGLEWVSTISGSAYSTYYADSVKGRFT |
| | hIL-2 | ISRDNSKSTLYLQMNSLRAEDTAVYYCAREIFSDYWGLGTLVTVSSASTKGPSVEPLAPCSRSTSESTA |
| | (F42I/I92D) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLDLQMILNGINNYKNPKLTRMLT |
| | | IKFYMPKKATELKHLQCLEEELKPLEEVLNLAQSKNFHLRPRDLISNINVDVLELKGSETTFMCEYADE |
| | | TATIVEFLNRWITFCQSIISTLT |
| 514 | A2-hIgG4-df- | DVQLVESGGGLVQPGGSLRLSCAASGFTFDISAMSWVRQAPGKGLEWVSTISGSAYSTYYADSVKGRET |
| | hIL-2 | ISRDNSKSTLYLQMNSLRAEDTAVYYCAREIFSDYWGLGTLVTVSSASTKGPSVFPLAPCSRSTSESTA |
| | (F42Q/I92D) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLDLQMILNGINNYKNPKLTRMLT |
| | | QKFYMPKKATELKHLQCLEEELKPLEEVLNLAQSKNFHLRPRDLISNINVDVLELKGSETTEMCEYADE |
| | | TATIVEFLNRWITFCQSIISTLT |
| 515 | A2-hIgG4-df- | DVQLVESGGGLVQPGGSLRLSCAASGFTFDISAMSWVRQAPGKGLEWVSTISGSAYSTYYADSVKGRET |
| | hIL-2 | ISRDNSKSTLYLQMNSLRAEDTAVYYCAREIFSDYWGLGTLVTVSSASTKGPSVFPLAPCSRSTSESTA |
| | (F42T/192D) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLDLQMILNGINNYKNPKLTRMLT |
| | | TKFYMPKKATELKHLQCLEEELKPLEEVLNLAQSKNFHLRPRDLISNINVDVLELKGSETTEMCEYADE |
| | | TATIVEFLNRWITFCQSIISTLT |
| 516 | A2-hIgG4-df- | DVQLVESGGGLVQPGGSLRLSCAASGFTFDISAMSWVRQAPGKGLEWVSTISGSAYSTYYADSVKGRFT |
| | hIL-2 | ISRDNSKSTLYLQMNSLRAEDTAVYYCAREIFSDYWGLGTLVTVSSASTKGPSVFPLAPCSRSTSESTA |
| | (F42W/I92D) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLDLOMILNGINNYKNPKLTRMLT |
| | | WKFYMPKKATELKHLQCLEEELKPLEEVLNLAQSKNFHLRPRDLISNINVDVLELKGSETTEMCEYADE |
| | | TATIVEFLNRWITFCQSIISTLT |
| 517 | A2-hIgG4-df- | DVQLVESGGGLVQPGGSLRLSCAASGFTFDISAMSWVRQAPGKGLEWVSTISGSAYSTYYADSVKGRET |
| | hIL-2 | ISRDNSKSTLYLQMNSLRAEDTAVYYCAREIFSDYWGLGTLVTVSSASTKGPSVFPLAPCSRSTSESTA |
| | (R38E/D84K) HC | ALGCLVKDYFPEPVTVSWNSGALTSGVHTFPAVLOSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQENWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLDLQMILNGINNYKNPKLTEMLT |
| | | FKFYMPKKATELKHLQCLEEELKPLEEVLNLAQSKNFHLRPRKLISNINVIVLELKGSETTEMCEYADE |
| | | TATIVEFLNRWITFCQSIISTLT |
| 518 | A2-hIgG4-df- | DVQLVESGGGLVQPGGSLRLSCAASGFTFDISAMSWVRQAPGKGLEWVSTISGSAYSTYYADSVKGRET |
| | hIL-2 | ISRDNSKSTLYLQMNSLRAEDTAVYYCAREIFSDYWGLGTLVTVSSASTKGPSVFPLAPCSRSTSESTA |
| | (R38E/I92K) HC | ALGCLVKDYFPEPVTVSWNSGALTSGVHTFPAVLQSSGLYSLSSVVTVPSSSLGTKTYTCNVDHKPSNT |
| | hIL-2 in | KVDKRVESKYGPPCPPCPAPEFLGGPSVFLFPPKPKDTLMISRTPEVTCVVVDVSQEDPEVQFNWYVDG |
| | italics | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPTSSSTKKTQLQLEHLLLDLOMILNGINNYKNPKLTEMLT |
| | | FKFYMPKKATELKHLQCLEEELKPLEEVLNLAQSKNFHLRPRDLISNINVKVLELKGSETTEMCEYADE |
| | | TATIVEFLNRWITFCQSIISTLT |
| 519 | C51E6-5-hIgG4- | QVQLVQSGSELKKPGASVKVSCKASGYSLYGTSMHWVRQAPGQGLEWMGYISPFTGRATYAQGFTGREV |
| | df-hIL-2 | FSLDTSVSTAYLQISSLKAEDTAVYYCARDYDYRYYYAMDYWGQGTTVTVSSASTKGPSVEPLAPCSRS |
| | (D20A/R38E) HC | TSESTAALGCLVKDYFPEPVTVSWNSGALTSGVHTFPAVLQSSGLYSLSSVVTVPSSSLGTKTYTCNVD |
| | hIL-2 in | HKPSNTKVDKRVESKYGPPCPPCPAPEFLGGPSVFLFPPKPKDTLMISRTPEVTCVVVDVSQEDPEVQF |
| | italics | NWYVDGVEVHNAKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDGSFFLYSRLTVD |
| | | KSRWQEGNVFSCSVMHEALHNHYTQKSLSLSLGKAPTSSSTKKTQLQLEHLLLALQMILNGINNYKNPK |
| | | LTEMLTFKFYMPKKATELKHLQCLEEELKPLEEVLNLAQSKNFHLRPRDLISNINVIVLELKGSETTEM |
| | | CEYADETATIVEFLNRWITFCQSIISTLT |
| 520 | C51E6-5-hIgG4- | QVQLVQSGSELKKPGASVKVSCKASGYSLYGTSMHWVRQAPGQGLEWMGYISPFTGRATYAQGFTGREV |
| | LE-df-hIL-2 | FSLDTSVSTAYLQISSLKAEDTAVYYCARDYDYRYYYAMDYWGQGTTVTVSSASTKGPSVFPLAPCSRS |
| | (T3A/D20A/R38E/ | TSESTAALGCLVKDYFPEPVTVSWNSGALTSGVHTFPAVLQSSGLYSLSSVVTVPSSSLGTKTYTCNVD |
| | C125A) HC | HKPSNTKVDKRVESKYGPPCPPCPAPEFEGGPSVFLFPPKPKDTLMISRTPEVTCVVVDVSQEDPEVQF |
| | hIL-2 in | NWYVDGVEVHNAKTKPREEQFNSTYRVVSVLTVLHQDWLNGKEYKCKVSNKGLPSSIEKTISKAKGQPR |
| | italics | EPQVYTLPPSQEEMTKNQVSLTCLVKGFYPSDIAVEWESNGQPENNYKTTPPVLDSDGSFFLYSRLTVD |
| | | KSRWQEGNVFSCSVMHEALHNHYTQKSLSLSLGKAPASSSTKKTQLQLEHLLLALOMILNGINNYKNPK |
| | | LTEMLTFKFYMPKKATELKHLQCLEEELKPLEEVLNLAQSKNFHLRPRDLISNINVIVLELKGSETTEM |
| | | CEYADETATIVEFLNRWITFAQSIISTLT |
| 521 | C51E6-5-hIgG4- | QVQLVQSGSELKKPGASVKVSCKASGYSLYGTSMHWVRQAPGQGLEWMGYISPFTGRATYAQGFTGREV |
| | LAGA-df-hIL-2 | FSLDTSVSTAYLQISSLKAEDTAVYYCARDYDYRYYYAMDYWGQGTTVTVSSASTKGPSVEPLAPCSRS |
| | (T3A/D20A/R38E/ | TSESTAALGCLVKDYFPEPVTVSWNSGALTSGVHTFPAVLQSSGLYSLSSVVTVPSSSLGTKTYTCNVD |
| | C125A) HC | HKPSNTKVDKRVESKYGPPCPPCPAPEFAGAPSVELFPPKPKDTLMISRTPEVTCVVVDVSQEDPEVQF |
| | hIL-2 in | NWYVDGVEVHNAKTKPREEQFNSTYRVVSVLTVLHQDWLNGKEYKCKVSNKGLPSSIEKTISKAKGQPR |
| | italics | EPQVYTLPPSQEEMTKNQVSLTCLVKGFYPSDIAVEWESNGQPENNYKTTPPVLDSDGSFFLYSRLTVD |
| | | KSRWQEGNVFSCSVMHEALHNHYTQKSLSLSLGKAPASSSTKKTQLQLEHLLLALQMILNGINNYKNPK |
| | | LTEMLTFKFYMPKKATELKHLQCLEEELKPLEEVLNLAQSKNFHLRPRDLISNINVIVLELKGSETTEM |
| | | CEYADETATIVEFLNRWITFAQSIISTLT |
| 522 | 1H3-hIgG1- | EVOLVESGGGLVQPGRSLKLSCAVSGFTESDYAMAWVRQAPKKGLEWVATISYDGSRTYYRDSVKGRFT |
| | LAGA-L6-hIL-2 | ISRDNAKITLYLQMDSLRSEDTATYYCARHGSGYFDYWGQGVMVTVSSASTKGPSVFPLAPSSKSTSGG |
| | (T3A/D20A/R38E/ | TAALGCLVKDYFPEPVTVSWNSGALTSGVHTFPAVLQSSGLYSLSSVVTVPSSSLGTQTYICNVNHKPS |
| | C125A) | NTKVDKKVEPKSCDKTHTCPPCPAPELAGAPSVELFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSAPASSSTKKTQLQLEHLLLALQMILNGINN |
| | | YKNPKLTEMLTFKFYMPKKATELKHLQCLEEELKPLEEVLNLAQSKNFHLRPRDLISNINVIVLELKGS |
| | | ETTFMCEYADETATIVEFLNRWITFAQSIISTLT |
| 523 | 2A3-hKappa LC | EIVLTQSPGTLSLSPGERATLSCRASQSIGRSFLAWYQQKPGQAPRLLIYDASTRAADIPARFSGSGSG |
| | | TDFTLTISSLEPEDFAVYYCQQYYDWPPLSFGGGTKVEIKRTVAAPSVFIFPPSDEQLKSGTASVVCLL |
| | | NNFYPREAKVQWKVDNALQSGNSQESVTEQDSKDSTYSLSSTLTLSKADYEKHKVYACEVTHQGLSSPV |
| | | TKSENRGEC |
| 524 | 1H9-hIgG4-df- | EVQLLESGGGLVQPGGSLRLSCVGSGENLKDYCMTWVRQAPGKGLEWVSAIVYSGGSTYYADSVKGRET |
| | hIL-2 | ISRDNSKNTLYLQMNSLRAEDTAVYYCAKYTRGSYFYDAMDVWGQGTTVTVSSASTKGPSVFPLAPCSR |
| | (D20A/R38E) HC | STSESTAALGCLVKDYFPEPVTVSWNSGALTSGVHTFPAVLQSSGLYSLSSVVTVPSSSLGTKTYTCNV |
| | hIL-2 in | DHKPSNTKVDKRVESKYGPPCPPCPAPEFLGGPSVFLFPPKPKDTLMISRTPEVTCVVVDVSQEDPEVQ |
| | italics | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPTSSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFCQSIISTLT |
| 525 | 1H9-hkappa LC | EIVLTQSPGTLSLSPGERATLSCRASQSIGRSFLAWYQQKPGQAPRLLIYDASTRAADIPDRESGSGSG |
| | | TDFTLTINRLEPEDFAVYYCQQYYDWPPLTFGGGTKVEIKRTVAAPSVFIFPPSDEQLKSGTASVVCLL |
| | | NNFYPREAKVQWKVDNALQSGNSQESVTEQDSKDSTYSLSSTLTLSKADYEKHKVYACEVTHQGLSSPV |
| | | TKSENRGEC |
| 526 | 1D5-hIgG4-df- | EVQLLESGGGLVQPGGSLRLSCVGSGENFKDYCMTWVRQAPGKGLEWVSAIVYSGGSTYYADSVKGRFT |
| | hIL-2 | ISRDNSKNTLYLQMNSLRAEDTAVYYCAKYTRGSYFYDAMDVWGQGTTVTVSSASTKGPSVFPLAPCSR |
| | (D20A/R38E) HC | STSESTAALGCLVKDYFPEPVTVSWNSGALTSGVHTFPAVLQSSGLYSLSSVVTVPSSSLGTKTYTCNV |
| | hIL-2 in | DHKPSNTKVDKRVESKYGPPCPPCPAPEFLGGPSVELFPPKPKDTLMISRTPEVTCVVVDVSQEDPEVQ |
| | italics | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPTSSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFCQSIISTLT |
| 527 | 1D5-hKappa LC | EIVLTQSPGTLSLSPGERATLSCRASQSIGRSFLAWYQQKPGQAPRLLIYDASTRATDIPDRESGSGSG |
| | | TEFTLTISSLQSEDFAVYYCQQYYDWPPLTFGGGTKVEIKRTVAAPSVFIFPPSDEQLKSGTASVVCLL |
| | | NNFYPREAKVQWKVDNALQSGNSQESVTEQDSKDSTYSLSSTLTLSKADYEKHKVYACEVTHQGLSSPV |
| | | TKSENRGEC |
| 528 | 1D5-hIgG4-LE- | EVQLLESGGGLVQPGGSLRLSCVGSGENFKDYCMTWVRQAPGKGLEWVSAIVYSGGSTYYADSVKGRET |
| | df-hIL-2 | ISRDNSKNTLYLQMNSLRAEDTAVYYCAKYTRGSYFYDAMDVWGQGTTVTVSSASTKGPSVEPLAPCSR |
| | (T3A/D20A/R38E/ | STSESTAALGCLVKDYFPEPVTVSWNSGALTSGVHTFPAVLQSSGLYSLSSVVTVPSSSLGTKTYTCNV |
| | C125A) HC | DHKPSNTKVDKRVESKYGPPCPPCPAPEFEGGPSVFLFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFAQSIISTLT |
| 529 | 1D5-hIgG4- | EVQLLESGGGLVQPGGSLRLSCVGSGENFKDYCMTWVRQAPGKGLEWVSAIVYSGGSTYYADSVKGRFT |
| | LAGA-df-hIL-2 | ISRDNSKNTLYLQMNSLRAEDTAVYYCAKYTRGSYFYDAMDVWGQGTTVTVSSASTKGPSVEPLAPCSR |
| | (T3A/D20A/R38E/ | STSESTAALGCLVKDYFPEPVTVSWNSGALTSGVHTFPAVLQSSGLYSLSSVVTVPSSSLGTKTYTCNV |
| | C125A) HC | DHKPSNTKVDKRVESKYGPPCPPCPAPEFAGAPSVFLFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGOP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLALQMILNGINNYKNP |
| | | KLTEMLTFKFYMPKKATELKHLQCLEEELKPLEEVLNLAQSKNFHLRPRDLISNINVIVLELKGSETTE |
| | | MCEYADETATIVEFLNRWITFAQSIISTLT |
| 530 | 2H7-hIgG1-df- | EVQLLESGGGLVQPGGSLRLSCAASGFTFKDYCMTWVRQAPGKGLEWVSAIVYSGGSTYYADSVKGRET |
| | hIL-2 | ISRDNSKNTLYLQMNNLRAEDTAVYYCAKYTRASYFYDAMDVWGQGTTVTVSSASTKGPSVFPLAPSSK |
| | (T3A/D20A/R38E/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELLGGPSVELFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLALQMILNGINNY |
| | | KNPKLTEMLTFKFYMPKKATELKHLQCLEEELKPLEEVLNLAQSKNFHLRPRDLISNINVIVLELKGSE |
| | | TTFMCEYADETATIVEFLNRWITFAQSIISTLT |
| 531 | 2H7-hIgG1-LE- | EVQLLESGGGLVQPGGSLRLSCAASGFTFKDYCMTWVRQAPGKGLEWVSAIVYSGGSTYYADSVKGRFT |
| | df-hIL-2 | ISRDNSKNTLYLQMNNLRAEDTAVYYCAKYTRASYFYDAMDVWGQGTTVTVSSASTKGPSVFPLAPSSK |
| | (T3A/D20A/R38E/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELEGGPSVFLFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLALQMILNGINNY |
| | | KNPKLTEMLTFKFYMPKKATELKHLQCLEEELKPLEEVLNLAQSKNFHLRPRDLISNINVIVLELKGSE |
| | | TTFMCEYADETATIVEFLNRWITFAQSIISTLT |
| 532 | H7-767 HC | EVQLLESGGGLVQPGGSLRLSCAASGFTEKSYAMHWVRQAPGKGLEWVSAIVYSGGSTYYADSVKGRET |
| | | ISRDNSKNTLYLQMNSLRAEDTAVYYCAKYDRASYFYDAMDVWGQGTTVTVSSASTKGPSVFPLAPSSK |
| | | STSGGTAALGCLVKDYFPEPVTVSWNSGALTSGVHTFPAVLOSSGLYSLSSVVTVPSSSLGTQTYICNV |
| | | NHKPSNTKVDKKVEPKSCDKTHTCPPCPAPELAGAPSVELFPPKPKDTLMISRTPEVTCVVVDVSHEDP |
| | | EVKENWYVDGVEVHNAKTKPREEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLALQMILNGINNY |
| | | KNPKLTEMLTFKFYMPKKATELKHLQCLEEELKPLEEVLNLAQSKNFHLRPRDLISNINVIVLELKGSE |
| | | TTEMCEYADETATIVEFLNRWITFAQSIISTLT |
| 533 | 2H7-hIgG1-LE- | EVQLLESGGGLVQPGGSLRLSCAASGFTFKDYCMTWVRQAPGKGLEWVSAIVYSGGSTYYADSVKGRET |
| | df-hIL-2 | ISRDNSKNTLYLQMNNLRAEDTAVYYCAKYTRASYFYDAMDVWGQGTTVTVSSASTKGPSVFPLAPSSK |
| | (T3A/R38E/D84K/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELEGGPSVFLFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLDLOMILNGINNY |
| | | KNPKLTEMLTFKFYMPKKATELKHLQCLEEELKPLEEVLNLAQSKNFHLRPRKLISNINVIVLELKGSE |
| | | TTFMCEYADETATIVEFLNRWITFAQSIISTLT |
| 534 | 2H7-hIgG4-df- | EVQLLESGGGLVQPGGSLRLSCAASGFTFKDYCMTWVRQAPGKGLEWVSAIVYSGGSTYYADSVKGRFT |
| | hIL-2 | ISRDNSKNTLYLQMNNLRAEDTAVYYCAKYTRASYFYDAMDVWGQGTTVTVSSASTKGPSVEPLAPCSR |
| | (T3A/R38E/D84K/ | STSESTAALGCLVKDYFPEPVTVSWNSGALTSGVHTFPAVLOSSGLYSLSSVVTVPSSSLGTKTYTCNV |
| | C125A) HC | DHKPSNTKVDKRVESKYGPPCPPCPAPEFLGGPSVELFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLDLOMILNGINNYKNP |
| | | KLTEMLTFKFYMPKKATELKHLQCLEEELKPLEEVLNLAQSKNFHLRPRKLISNINVIVLELKGSETTE |
| | | MCEYADETATIVEFLNRWITFAQSIISTLT |
| 535 | 2H7-hIgG1-df- | EVQLLESGGGLVQPGGSLRLSCAASGFTFKDYCMTWVRQAPGKGLEWVSAIVYSGGSTYYADSVKGRFT |
| | hIL-2 | ISRDNSKNTLYLQMNNLRAEDTAVYYCAKYTRASYFYDAMDVWGQGTTVTVSSASTKGPSVFPLAPSSK |
| | (T3A/R38E/192K/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELLGGPSVFLFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLDLOMILNGINNY |
| | | KNPKLTEMLTFKFYMPKKATELKHLQCLEEELKPLEEVLNLAQSKNFHLRPRDLISNINVKVLELKGSE |
| | | TTFMCEYADETATIVEFLNRWITFAQSIISTLT |
| 536 | 2H7-hIgG1-LE- | EVQLLESGGGLVQPGGSLRLSCAASGFTFKDYCMTWVRQAPGKGLEWVSAIVYSGGSTYYADSVKGRET |
| | df-hIL-2 | ISRDNSKNTLYLQMNNLRAEDTAVYYCAKYTRASYFYDAMDVWGQGTTVTVSSASTKGPSVFPLAPSSK |
| | (T3A/R38E/192K/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELEGGPSVELFPPKPKDTLMISRTPEVTCVVVDVSHEDP |
| | | EVKFNWYVDGVEVHNAKTKPREEQYNSTYRVVSVLTVLHQDWLNGKEYKCKVSNKALPAPIEKTISKAK |
| | hIL-2 in | GQPREPQVYTLPPSRDELTKNQVSLTCLVKGFYPSDIAVEWESNGQPENNYKTTPPVLDSDGSFFLYSK |
| | italics | LTVDKSRWQQGNVFSCSVMHEALHNHYTQKSLSLSPGKAPASSSTKKTQLQLEHLLLDLQMILNGINNY |
| | | KNPKLTEMLTFKFYMPKKATELKHLQCLEEELKPLEEVLNLAQSKNFHLRPRDLISNINVKVLELKGSE |
| | | TTFMCEYADETATIVEFLNRWITFAQSIISTLT |
| 537 | 2H7-hIgG4-df- | EVQLLESGGGLVQPGGSLRLSCAASGFTFKDYCMTWVRQAPGKGLEWVSAIVYSGGSTYYADSVKGRET |
| | hIL-2 | ISRDNSKNTLYLQMNNLRAEDTAVYYCAKYTRASYFYDAMDVWGQGTTVTVSSASTKGPSVEPLAPCSR |
| | (T3A/R38E/192K/ | STSESTAALGCLVKDYFPEPVTVSWNSGALTSGVHTFPAVLQSSGLYSLSSVVTVPSSSLGTKTYTCNV |
| | C125A) HC | DHKPSNTKVDKRVESKYGPPCPPCPAPEFLGGPSVFLFPPKPKDTLMISRTPEVTCVVVDVSQEDPEVQ |
| | hIL-2 in | FNWYVDGVEVHNAKTKPREEQFNSTYRVVSVLTVLHQDWLNGKEYKCKVSNKGLPSSIEKTISKAKGQP |
| | italics | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLGKAPASSSTKKTQLQLEHLLLDLQMILNGINNYKNP |
| | | KLTEMLTFKFYMPKKATELKHLQCLEEELKPLEEVLNLAQSKNFHLRPRDLISNINVKVLELKGSETTE |
| | | MCEYADETATIVEFLNRWITFAQSIISTLT |
| 538 | 2H7-hIgG1- | EVQLLESGGGLVQPGGSLRLSCAASGFTFKDYCMTWVRQAPGKGLEWVSAIVYSGGSTYYADSVKGRET |
| | LAGA-df-hIL2 | ISRDNSKNTLYLQMNNLRAEDTAVYYCAKYTRASYFYDAMDVWGQGTTVTVSSASTKGPSVFPLAPSSK |
| | (T3A/D20S/R38E/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL-2 in | EVKENWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLSLQMILNGINNY |
| | | KNPKLTEMLTFKFYMPKKATELKHLOCLEEELKPLEEVLNLAQSKNFHLRPRDLISNINVIVLELKGSE |
| | | TTFMCEYADETATIVEFLNRWITFAQSIISTLT |
| 539 | 2H7-hIgG1- | EVQLLESGGGLVQPGGSLRLSCAASGFTFKDYCMTWVRQAPGKGLEWVSAIVYSGGSTYYADSVKGRET |
| | LAGA-df-hIL2 | ISRDNSKNTLYLQMNNLRAEDTAVYYCAKYTRASYFYDAMDVWGQGTTVTVSSASTKGPSVFPLAPSSK |
| | (T3A/R38E/D84F/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLDLQMILNGINNY |
| | | KNPKLTEMLTFKFYMPKKATELKHLOCLEEELKPLEEVLNLAQSKNFHLRPRFLISNINVIVLELKGSE |
| | | TTFMCEYADETATIVEFLNRWITFAQSIISTLT |
| 540 | 2H7-hIgG1- | EVQLLESGGGLVQPGGSLRLSCAASGFTFKDYCMTWVRQAPGKGLEWVSAIVYSGGSTYYADSVKGRET |
| | LAGA-df-hIL2 | ISRDNSKNTLYLQMNNLRAEDTAVYYCAKYTRASYFYDAMDVWGQGTTVTVSSASTKGPSVFPLAPSSK |
| | (T3A/R38E/I92R/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLDLQMILNGINNY |
| | | KNPKLTEMLTFKFYMPKKATELKHLQCLEEELKPLEEVLNLAQSKNFHLRPRDLISNINVRVLELKGSE |
| | | TTFMCEYADETATIVEFLNRWITFAQSIISTLT |
| 541 | 2H7-hIgG1- | EVQLLESGGGLVQPGGSLRLSCAASGFTFKDYCMTWVRQAPGKGLEWVSAIVYSGGSTYYADSVKGRET |
| | LAGA-df-hIL2 | ISRDNSKNTLYLQMNNLRAEDTAVYYCAKYTRASYFYDAMDVWGQGTTVTVSSASTKGPSVFPLAPSSK |
| | (T3A/R38E/I92E/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLDLOMILNGINNY |
| | | KNPKLTEMLTFKFYMPKKATELKHLQCLEEELKPLEEVLNLAQSKNFHLRPRDLISNINVEVLELKGSE |
| | | TTFMCEYADETATIVEFLNRWITFAQSIISTLT |
| 542 | 2H7-hIgG1- | EVQLLESGGGLVQPGGSLRLSCAASGFTFKDYCMTWVRQAPGKGLEWVSAIVYSGGSTYYADSVKGRFT |
| | LAGA-df-hIL2 | ISRDNSKNTLYLQMNNLRAEDTAVYYCAKYTRASYFYDAMDVWGQGTTVTVSSASTKGPSVFPLAPSSK |
| | (T3A/R38E/192S/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLDLOMILNGINNY |
| | | KNPKLTEMLTFKFYMPKKATELKHLQCLEEELKPLEEVLNLAQSKNFHLRPRDLISNINVSVLELKGSE |
| | | TTFMCEYADETATIVEFLNRWITFAQSIISTLT |
| 543 | 2H7-hIgG1- | EVQLLESGGGLVQPGGSLRLSCAASGFTFKDYCMTWVRQAPGKGLEWVSAIVYSGGSTYYADSVKGRFT |
| | LAGA-df-hIL2 | ISRDNSKNTLYLQMNNLRAEDTAVYYCAKYTRASYFYDAMDVWGQGTTVTVSSASTKGPSVFPLAPSSK |
| | (T3A/R38E/192D/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLDLQMILNGINNY |
| | | KNPKLTEMLTFKFYMPKKATELKHLQCLEEELKPLEEVLNLAQSKNFHLRPRDLISNINVDVLELKGSE |
| | | TTFMCEYADETATIVEFLNRWITFAQSIISTLT |
| 544 | 2H7-hIgG1- | EVQLLESGGGLVQPGGSLRLSCAASGFTFKDYCMTWVRQAPGKGLEWVSAIVYSGGSTYYADSVKGRFT |
| | LAGA-df-hIL2 | ISRDNSKNTLYLQMNNLRAEDTAVYYCAKYTRASYFYDAMDVWGQGTTVTVSSASTKGPSVFPLAPSSK |
| | (T3A/H16E/R38E/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | hIL-2 in | EVKFNWYVDGVEVHNAKTKPREEQYNSTYRVVSVLTVLHQDWLNGKEYKCKVSNKALPAPIEKTISKAK |
| | italics | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEELLLDLQMILNGINNY |
| | | KNPKLTEMLTFKFYMPKKATELKHLQCLEEELKPLEEVLNLAQSKNFHLRPRDLISNINVIVLELKGSE |
| | | TTFMCEYADETATIVEFLNRWITFAQSIISTLT |
| 545 | 1H3-hIgG1-L6- | EQLVESGGGLVQPGRSLKLSCAVSGFTFSDYAMAWVRQAPKKGLEWVATISYDGSRTYYRDSVKGRET |
| | hIL-2 | ISRDNAKITLYLOMDSLRSEDTATYYCARHGSGYFDYWGQGVMVTVSSASTKGPSVFPLAPSSKSTSGG |
| | (T3A/D20A/R38E/ | TAALGCLVKDYFPEPVTVSWNSGALTSGVHTFPAVLOSSGLYSLSSVVTVPSSSLGTQTYICNVNHKPS |
| | C125A) HC | NTKVDKKVEPKSCDKTHTCPPCPAPELLGGPSVFLFPPKPKDTLMISRTPEVTCVVVDVSHEDPEVKEN |
| | Linker in | WYVDGVEVHNAKTKPREEQYNSTYRVVSVLTVLHQDWLNGKEYKCKVSNKALPAPIEKTISKAKGQPRE |
| | dashed | PQVYTLPPSRDELTKNQVSLTCLVKGFYPSDIAVEWESNGQPENNYKTTPPVLDSDGSFFLYSKLTVDK |
| | underline | SRWQQGNVFSCSVMHEALHNHYTQKSLSLSPGK 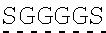 APASSSTKKTQLQLEHLL |
| | hIL-2 in | LALQMILNGINNYKNPKLTEMLTFKFYMPKKATELKHLQCLEEELKPLEEVLNLAQSKNFHLR |
| | italics | PRDLISNINVIVLELKGSETTEMCEYADETATIVEFLNRWITFAQSIISTLT |
| 546 | 1H3-hIgG1- | EVQLVESGGGLVQPGRSLKLSCAVSGFTFSDYAMAWVRQAPKKGLEWVATISYDGSRTYYRDSVKGRET |
| | LAGA-df-hIL-2 | ISRDNAKITLYLOMDSLRSEDTATYYCARHGSGYFDYWGQGVMVTVSSASTKGPSVFPLAPSSKSTSGG |
| | (T3A/D20A/R38E/ | TAALGCLVKDYFPEPVTVSWNSGALTSGVHTFPAVLQSSGLYSLSSVVTVPSSSLGTQTYICNVNHKPS |
| | C125A) HC | NTKVDKKVEPKSCDKTHTCPPCPAPELAGAPSVELFPPKPKDTLMISRTPEVTCVVVDVSHEDPEVKEN |
| | hIL-2 in | WYVDGVEVHNAKTKPREEQYNSTYRVVSVLTVLHQDWLNGKEYKCKVSNKALPAPIEKTISKAKGQPRE |
| | italics | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKAPASSSTKKTQLQLEHLLLALQMILNGINNYKNPKL |
| | | TEMLTFKFYMPKKATELKHLQCLEEELKPLEEVLNLAQSKNFHLRPRDLISNINVIVLELKGSETTEMC |
| | | EYADETATIVEFLNRWITFAQSIISTLT |
| 547 | C51E6-5- | QVQLVQSGSELKKPGASVKVSCKASGYSLYGTSMHWVRQAPGQGLEWMGYISPFTGRATYAQGFTGREV |
| | hIgG4/k-LE HC | FSLDTSVSTAYLQISSLKAEDTAVYYCARDYDYRYYYAMDYWGQGTTVTVSSASTKGPSVFPLAPCSRS |
| | | TSESTAALGCLVKDYFPEPVTVSWNSGALTSGVHTFPAVLQSSGLYSLSSVVTVPSSSLGTKTYTCNVD |
| | | HKPSNTKVDKRVESKYGPPCPPCPAPEFEGGPSVFLFPPKPKDTLMISRTPEVTCVVVDVSQEDPEVQF |
| | | NWYVDGVEVHNAKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDGSFFLYSRLTVD |
| | | KSRWQEGNVESCSVMHEALHNHYTQKSLSLSLG |
| 548 | C51E6-5- | QVQLVQSGSELKKPGASVKVSCKASGYSLYGTSMHWVRQAPGOGLEWMGYISPFTGRATYAQGFTGREV |
| | hIgG4/k-LAGA | FSLDTSVSTAYLQISSLKAEDTAVYYCARDYDYRYYYAMDYWGQGTTVTVSSASTKGPSVEPLAPCSRS |
| | HC | TSESTAALGCLVKDYFPEPVTVSWNSGALTSGVHTFPAVLQSSGLYSLSSVVTVPSSSLGTKTYTCNVD |
| | | HKPSNTKVDKRVESKYGPPCPPCPAPEFAGAPSVELFPPKPKDTLMISRTPEVTCVVVDVSQEDPEVQF |
| | | NWYVDGVEVHNAKTKPREEQFNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKTTPPVLDSDGSFFLYSRLTVD |
| | | KSRWQEGNVFSCSVMHEALHNHYTQKSLSLSLG |
| 549 | C51E6-5- | QVQLVQSGSELKKPGASVKVSCKASGYSLYGTSMHWVRQAPGQGLEWMGYISPFTGRATYAQGFTGREV |
| | hIgG4/k-LEPG | FSLDTSVSTAYLQISSLKAEDTAVYYCARDYDYRYYYAMDYWGQGTTVTVSSASTKGPSVFPLAPCSRS |
| | HC | TSESTAALGCLVKDYFPEPVTVSWNSGALTSGVHTFPAVLQSSGLYSLSSVVTVPSSSLGTKTYTCNVD |
| | | HKPSNTKVDKRVESKYGPPCPPCPAPEFEGGPSVELFPPKPKDTLMISRTPEVTCVVVDVSQEDPEVQF |
| | | NWYVDGVEVHNAKTKPREEQFNSTYRVVSVLTVLHQDWLNGKEYKCKVSNKGLGSSIEKTISKAKGQPR |
| | | EPQVYTLPPSQEEMTKNQVSLTCLVKGFYPSDIAVEWESNGQPENNYKTTPPVLDSDGSFFLYSRLTVD |
| | | KSRWQEGNVFSCSVMHEALHNHYTQKSLSLSLG |
| 550 | C51E6-5- | QVQLVQSGSELKKPGASVKVSCKASGYSLYGTSMHWVRQAPGQGLEWMGYISPFTGRATYAQGFTGREV |
| | hIgG4/k-df- | FSLDTSVSTAYLQISSLKAEDTAVYYCARDYDYRYYYAMDYWGQGTTVTVSSASTKGPSVFPLAPCSRS |
| | hIL-2 | TSESTAALGCLVKDYFPEPVTVSWNSGALTSGVHTFPAVLQSSGLYSLSSVVTVPSSSLGTKTYTCNVD |
| | (T3A/D20A/R38E/ | HKPSNTKVDKRVESKYGPPCPPCPAPEFLGGPSVELFPPKPKDTLMISRTPEVTCVVVDVSQEDPEVQF |
| | C125A) HC | NWYVDGVEVHNAKTKPREEQFNSTYRVVSVLTVLHQDWLNGKEYKCKVSNKGLPSSIEKTISKAKGQPR |
| | hIL-2 in | EPQVYTLPPSQEEMTKNQVSLTCLVKGFYPSDIAVEWESNGQPENNYKTTPPVLDSDGSFFLYSRLTVD |
| | italics | KSRWQEGNVFSCSVMHEALHNHYTQKSLSLSLGKAPASSSTKKTQLQLEHLLLALQMILNGINNYKNPK |
| | | LTEMLTFKFYMPKKATELKHLQCLEEELKPLEEVLNLAQSKNFHLRPRDLISNINVIVLELKGSETTEM |
| | | CEYADETATIVEFLNRWITFAQSIISTLT |
| 551 | C51E6-5- | QVQLVQSGSELKKPGASVKVSCKASGYSLYGTSMHWVRQAPGQGLEWMGYISPFTGRATYAQGFTGREV |
| | hIgG4/k-LEPG- | FSLDTSVSTAYLQISSLKAEDTAVYYCARDYDYRYYYAMDYWGQGTTVTVSSASTKGPSVEPLAPCSRS |
| | hIL-2 | TSESTAALGCLVKDYFPEPVTVSWNSGALTSGVHTFPAVLQSSGLYSLSSVVTVPSSSLGTKTYTCNVD |
| | (T3A/D20A/R38E/ | HKPSNTKVDKRVESKYGPPCPPCPAPEFEGGPSVFLFPPKPKDTLMISRTPEVTCVVVDVSQEDPEVQF |
| | C125A) HC | NWYVDGVEVHNAKTKPREEQFNSTYRVVSVLTVLHQDWLNGKEYKCKVSNKGLGSSIEKTISKAKGQPR |
| | hIL-2 in | EPQVYTLPPSQEEMTKNQVSLTCLVKGFYPSDIAVEWESNGQPENNYKTTPPVLDSDGSFFLYSRLTVD |
| | italics | KSRWQEGNVFSCSVMHEALHNHYTQKSLSLSLGKAPASSSTKKTQLQLEHLLLALQMILNGINNYKNPK |
| | | LTEMLTFKFYMPKKATELKHLQCLEEELKPLEEVLNLAQSKNFHLRPRDLISNINVIVLELKGSETTEM |
| | | CEYADETATIVEFLNRWITFAQSIISTLT |
| 552 | A2-hIgG4/k-LE | DVQLVESGGGLVQPGGSLRLSCAASGFTFDISAMSWVRQAPGKGLEWVSTISGSAYSTYYADSVKGRFT |
| | HC | ISRDNSKSTLYLQMNSLRAEDTAVYYCAREIFSDYWGLGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNT |
| | | KVDKRVESKYGPPCPPCPAPEFeGGPSVFLFPPKPKDTLMISRTPEVTCVVVDVSQEDPEVQFNWYVDG |
| | | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLG |
| 553 | A2-hIgG4/k- | DVQLVESGGGLVQPGGSLRLSCAASGFTFDISAMSWVRQAPGKGLEWVSTISGSAYSTYYADSVKGRFT |
| | LAGA HC | ISRDNSKSTLYLQMNSLRAEDTAVYYCAREIFSDYWGLGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNT |
| | | KVDKRVESKYGPPCPPCPAPEFaGaPSVFLFPPKPKDTLMISRTPEVTCVVVDVSQEDPEVQFNWYVDG |
| | | VEVHNAKTKPREEQFNSTYRVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLG |
| 554 | A2-hIgG4/k- | DVQLVESGGGLVQPGGSLRLSCAASGFTFDISAMSWVRQAPGKGLEWVSTISGSAYSTYYADSVKGRFT |
| | LEPG HC | ISRDNSKSTLYLQMNSLRAEDTAVYYCAREIFSDYWGLGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNT |
| | | KVDKRVESKYGPPCPPCPAPEFeGGPSVFLFPPKPKDTLMISRTPEVTCVVVDVSQEDPEVQFNWYVDG |
| | | VEVHNAKTKPREEQFNSTYRVVSVLTVLHQDWLNGKEYKCKVSNKGLgSSIEKTISKAKGQPREPQVYT |
| | | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLG |
| 555 | A2-hIgG4/k-df- | DVQLVESGGGLVQPGGSLRLSCAASGFTFDISAMSWVRQAPGKGLEWVSTISGSAYSTYYADSVKGRFT |
| | hIL-2 | ISRDNSKSTLYLQMNSLRAEDTAVYYCAREIFSDYWGLGTLVTVSSASTKGPSVFPLAPCSRSTSESTA |
| | (T3A/D20A/R38E/ | ALGCLVKDYFPEPVTVSWNSGALTSGVHTFPAVLQSSGLYSLSSVVTVPSSSLGTKTYTCNVDHKPSNT |
| | C125A) HC | KVDKRVESKYGPPCPPCPAPEFLGGPSVFLFPPKPKDTLMISRTPEVTCVVVDVSQEDPEVQFNWYVDG |
| | hIL-2 in | VEVHNAKTKPREEQFNSTYRVVSVLTVLHQDWLNGKEYKCKVSNKGLPSSIEKTISKAKGQPREPQVYT |
| | italics | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPASSSTKKTQLQLEHLLLALQMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFAQSIISTLT |
| 556 | A2-hIgG4/k-LE- | DVQLVESGGGLVQPGGSLRLSCAASGFTFDISAMSWVRQAPGKGLEWVSTISGSAYSTYYADSVKGRET |
| | df-hIL-2 | ISRDNSKSTLYLQMNSLRAEDTAVYYCAREIFSDYWGLGTLVTVSSASTKGPSVFPLAPCSRSTSESTA |
| | (T3A/D20A/R38E/ | ALGCLVKDYFPEPVTVSWNSGALTSGVHTFPAVLQSSGLYSLSSVVTVPSSSLGTKTYTCNVDHKPSNT |
| | C125A) HC | KVDKRVESKYGPPCPPCPAPEFEGGPSVFLFPPKPKDTLMISRTPEVTCVVVDVSQEDPEVQFNWYVDG |
| | hIL-2 in | VEVHNAKTKPREEQFNSTYRVVSVLTVLHQDWLNGKEYKCKVSNKGLPSSIEKTISKAKGQPREPQVYT |
| | italics | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPASSSTKKTQLQLEHLLLALQMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFAQSIISTLT |
| 557 | A2-hIgG4/k- | DVQLVESGGGLVQPGGSLRLSCAASGFTFDISAMSWVRQAPGKGLEWVSTISGSAYSTYYADSVKGRFT |
| | LAGA-df-hIL-2 | ISRDNSKSTLYLQMNSLRAEDTAVYYCAREIFSDYWGLGTLVTVSSASTKGPSVFPLAPCSRSTSESTA |
| | (T3A/D20A/R38E/ | ALGCLVKDYFPEPVTVSWNSGALTSGVHTFPAVLQSSGLYSLSSVVTVPSSSLGTKTYTCNVDHKPSNT |
| | C125A) HC | KVDKRVESKYGPPCPPCPAPEFAGAPSVELFPPKPKDTLMISRTPEVTCVVVDVSQEDPEVQFNWYVDG |
| | hIL-2 in | VEVHNAKTKPREEQFNSTYRVVSVLTVLHQDWLNGKEYKCKVSNKGLPSSIEKTISKAKGQPREPQVYT |
| | italics | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPASSSTKKTQLQLEHLLLALQMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFAQSIISTLT |
| 558 | A2-hIgG4/k- | DVQLVESGGGLVQPGGSLRLSCAASGFTFDISAMSWVRQAPGKGLEWVSTISGSAYSTYYADSVKGRET |
| | LEPG-df-hIL-2 | ISRDNSKSTLYLQMNSLRAEDTAVYYCAREIFSDYWGLGTLVTVSSASTKGPSVFPLAPCSRSTSESTA |
| | (T3A/D20A/R38E/ | ALGCLVKDYFPEPVTVSWNSGALTSGVHTFPAVLQSSGLYSLSSVVTVPSSSLGTKTYTCNVDHKPSNT |
| | C125A) HC | KVDKRVESKYGPPCPPCPAPEFEGGPSVFLFPPKPKDTLMISRTPEVTCVVVDVSQEDPEVQFNWYVDG |
| | hIL-2 in | VEVHNAKTKPREEQFNSTYRVVSVLTVLHQDWLNGKEYKCKVSNKGLGSSIEKTISKAKGQPREPQVYT |
| | italics | LPPSQEEMTKNQVSLTCLVKGFYPSDIAVEWESNGQPENNYKTTPPVLDSDGSFFLYSRLTVDKSRWQE |
| | | GNVFSCSVMHEALHNHYTQKSLSLSLGKAPASSSTKKTQLQLEHLLLALQMILNGINNYKNPKLTEMLT |
| | | FKFYMPKKATELKHLQCLEEELKPLEEVLNLAQSKNFHLRPRDLISNINVIVLELKGSETTEMCEYADE |
| | | TATIVEFLNRWITFAQSIISTLT |
| 559 | Anti-hPD-1 #1 | QVQLVESGGGVVQPGRSLRLDCKASGITFSNSGMHWVRQAPGKGLEWVAVIWYDGSKRYYADSVKGRFT |
| | HC | ISRDNSKNTLFLQMNSLRAEDTAVYYCATNDDYWGQGTLVTVSSASTKGPSVFPLAPCSRSTSESTAAL |
| | | GCLVKDYFPEPVTVSWNSGALTSGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCVVVDVSQEDPEVQENWYVDGVE |
| | | VHNAKTKPREEQFNSTYRVVSVLTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVLDSDGSFFLYSRLTVDKSRWQEGN |
| | | VFSCSVMHEALHNHYTQKSLSLSLG |
| 560 | Anti-CD20- | QVQLQQPGAELVKPGASVKMSCKASGYTFTSYNMHWVKQTPGRGLEWIGAIYPGNGDTSYNQKEKGKAT |
| | hIgG1/k HC | LTADKSSSTAYMQLSSLTSEDSAVYYCARSTYYGGDWYFNVWGAGTTVTVSAASTKGPSVFPLAPSSKS |
| | | TSGGTAALGCLVKDYFPEPVTVSWNSGALTSGVHTFPAVLQSSGLYSLSSVVTVPSSSLGTQTYICNVN |
| | | HKPSNTKVDKKVEPKSCDKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYKTTPPVLDSDGSFFLYSKL |
| | | TVDKSRWQQGNVFSCSVMHEALHNHYTQKSLSLSPGK |
| 561 | Anti-CD20- | QVQLQQPGAELVKPGASVKMSCKASGYTFTSYNMHWVKQTPGRGLEWIGAIYPGNGDTSYNQKFKGKAT |
| | hIgG1/k-LAGA | LTADKSSSTAYMQLSSLTSEDSAVYYCARSTYYGGDWYFNVWGAGTTVTVSAASTKGPSVFPLAPSSKS |
| | HC | TSGGTAALGCLVKDYFPEPVTVSWNSGALTSGVHTFPAVLQSSGLYSLSSVVTVPSSSLGTQTYICNVN |
| | | HKPSNTKVDKKVEPKSCDKTHTCPPCPAPELAGAPSVELFPPKPKDTLMISRTPEVTCVVVDVSHEDPE |
| | | VKFNWYVDGVEVHNAKTKPREEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNYKTTPPVLDSDGSFFLYSKL |
| | | TVDKSRWQQGNVFSCSVMHEALHNHYTQKSLSLSPG |
| 562 | Anti-CD20- | QIVLSQSPAILSASPGEKVTMTCRASSSVSYIHWFQQKPGSSPKPWIYATSNLASGVPVRESGSGSGTS |
| | hKappa LC | YSLTISRVEAEDAATYYCQQWTSNPPTFGGGTKLEIKRTVAAPSVFIFPPSDEQLKSGTASVVCLLNNF |
| | | YPREAKVQWKVDNALQSGNSQESVTEQDSKDSTYSLSSTLTLSKADYEKHKVYACEVTHQGLSSPVTKS |
| | | ENRGEC |
| 563 | 1H3-hIgG1- | EVOLVESGGGLVQPGRSLKLSCAVSGFTFSDYAMAWVRQAPKKGLEWVATISYDGSRTYYRDSVKGRFT |
| | LAGA-df-hIL-2 | ISRDNAKITLYLOMDSLRSEDTATYYCARHGSGYFDYWGQGVMVTVSSASTKGPSVFPLAPSSKSTSGG |
| | (T3A/C125A) HC | TAALGCLVKDYFPEPVTVSWNSGALTSGVHTFPAVLQSSGLYSLSSVVTVPSSSLGTQTYICNVNHKPS |
| | hIL-2 in | NTKVDKKVEPKSCDKTHTCPPCPAPELAGAPSVELFPPKPKDTLMISRTPEVTCVVVDVSHEDPEVKEN |
| | italics | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKAPASSSTKKTQLQLEHLLLDLQMILNGINNYKNPKL |
| | | TRMLTFKFYMPKKATELKHLQCLEEELKPLEEVLNLAQSKNFHLRPRDLISNINVIVLELKGSETTEMC |
| | | EYADETATIVEFLNRWITFAQSIISTLT |
| 564 | anti-mPD-1 | EVQLQESGPGLVKPSQSLSLTCSVTGYSITSSYRWNWIRKFPGNRLEWMGYINSAGISNYNPSLKRRIS |
| | RMP1-14 | ITRDTSKNQFFLQVNSVTTEDAATYYCARSDNMGTTPFTYWGQGTLVTVSSAKTTPPSVYPLAPGCGDT |
| | mIgG2b-N297A | TGSSVTLGCLVKGYFPESVTVTWNSGSLSSSVHTFPALLQSGLYTMSSSVTVPSSTWPSQTVTCSVAHP |
| | HC | ASSTTVDKKLEPSGPISTINPCPPCKECHKCPAPNLEGGPSVFIFPPNIKDVLMISLTPKVTCVVVDVS |
| | | EDDPDVQISWFVNNVEVHTAQTQTHREDYASTIRVVSTLPIQHQDWMSGKEFKCKVNNKDLPSPIERTI |
| | | SKIKGLVRAPQVYILPPPAEQLSRKDVSLTCLVVGENPGDISVEWTSNGHTEENYKDTAPVLDSDGSYF |
| | | IYSKLNMKTSKWEKTDSFSCNVRHEGLKNYYLKKTISRSPGK |
| 565 | anti-mPD-1 | EVQLQESGPGLVKPSQSLSLTCSVTGYSITSSYRWNWIRKFPGNRLEWMGYINSAGISNYNPSLKRRIS |
| | RMP1-14 | ITRDTSKNQFFLQVNSVTTEDAATYYCARSDNMGTTPFTYWGQGTLVTVSSAKTTPPSVYPLAPGCGDT |
| | mIgG2b-N297A- | TGSSVTLGCLVKGYFPESVTVTWNSGSLSSSVHTFPALLQSGLYTMSSSVTVPSSTWPSQTVTCSVAHP |
| | L6-hIL-2 | ASSTTVDKKLEPSGPISTINPCPPCKECHKCPAPNLEGGPSVFIFPPNIKDVLMISLTPKVTCVVVDVS |
| | (F42K/Y45R/V69R) | EDDPDVQISWFVNNVEVHTAQTQTHREDYASTIRVVSTLPIQHQDWMSGKEFKCKVNNKDLPSPIERTI |
| | HC | SKIKGLVRAPQVYILPPPAEQLSRKDVSLTCLVVGENPGDISVEWTSNGHTEENYKDTAPVLDSDGSYF |
| | linker in | IYSKLNMKTSKWEKTDSFSCNVRHEGLKNYYLKKTISRSPGK 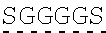 APTSSSTKKTQ |
| | dashed | LQLEHLLLDLQMILNGINNYKNPKLTRMLTKKFRMPKKATELKHLQCLEEELKPLEERLNL |
| | underline | AQSKNFHLRPRDLISNINVIVLELKGSETTEMCEYADETATIVEFLNRWITFCQSIISTLT |
| | hIL-2 in | |
| | italics | |
| 566 | anti-mPD-1 | DIVMTQGTLPNPVPSGESVSITCRSSKSLLYSDGKTYLNWYLQRPGQSPQLLIYWMSTRASGVSDRESG |
| | RMP1-14 mKappa | SGSGTDFTLKISGVEAEDVGIYYCQQGLEFPTFGGGTKLELKRADAAPTVSIFPPSSEQLTSGGASVVC |
| | LC | FLNNFYPKDINVKWKIDGSERQNGVLNSWTDQDSKDSTYSMSSTLTLTKDEYERHNSYTCEATHKTSTS |
| | | PIVKSENRNEC |
| 567 | anti-mPD-1 | EVOLVESGGGLVQPGRSLKLSCAASGFTFGDYSMAWVRQAPKRGLEWVANIIYDGSRTFYRDSVKGRFT |
| | RMP1-30 | ISRDNAKPTLYLQMDSLRPEDTATYYCATHNYPGYAMEAWGQGTSVTVSSAKTTPPSVYPLAPGCGDTT |
| | mIgG2b-N297A | GSSVTLGCLVKGYFPESVTVTWNSGSLSSSVHTFPALLQSGLYTMSSSVTVPSSTWPSQTVTCSVAHPA |
| | HC | SSTTVDKKLEPSGPISTINPCPPCKECHKCPAPNLEGGPSVFIFPPNIKDVLMISLTPKVTCVVVDVSE |
| | | DDPDVQISWFVNNVEVHTAQTQTHREDYASTIRVVSTLPIQHQDWMSGKEFKCKVNNKDLPSPIERTIS |
| | | KIKGLVRAPQVYILPPPAEQLSRKDVSLTCLVVGENPGDISVEWTSNGHTEENYKDTAPVLDSDGSYFI |
| | | YSKLNMKTSKWEKTDSFSCNVRHEGLKNYYLKKTISRSPGK |
| 568 | anti-mPD-1 | DTVLTQSPALPVSLGQRVNISCRATKSVSRYVHWYQQKSGQQPRLLIYTTSNLESGVPSRESGSGSGTD |
| | RMP1-30 mKappa | FTLTIDPVEADDIANYYCQQSNEIPYTFGAGTKLELKRADAAPTVSIFPPSSEQLTSGGASVVCELNNE |
| | LC | YPKDINVKWKIDGSERQNGVLNSWTDQDSKDSTYSMSSTLTLTKDEYERHNSYTCEATHKTSTSPIVKS |
| | | FNRNEC |
| 569 | anti-mPD-1 | EVQLVESGGGLVQPGRSLKLSCAASGFTFGDYSMAWVRQAPKRGLEWVANIIYDGSRTFYRDSVKGRFT |
| | RMP1-30 | ISRDNAKPTLYLQMDSLRPEDTATYYCATHNYPGYAMEAWGQGTSVTVSSAKTTPPSVYPLAPGCGDTT |
| | mIgG2b-N297A- | GSSVTLGCLVKGYFPESVTVTWNSGSLSSSVHTFPALLQSGLYTMSSSVTVPSSTWPSQTVTCSVAHPA |
| | L6-hIL-2 | SSTTVDKKLEPSGPISTINPCPPCKECHKCPAPNLEGGPSVFIFPPNIKDVLMISLTPKVTCVVVDVSE |
| | (F42K/Y45R/V69R) | DDPDVQISWFVNNVEVHTAQTQTHREDYASTIRVVSTLPIQHQDWMSGKEFKCKVNNKDLPSPIERTIS |
| | HC | KIKGLVRAPQVYILPPPAEQLSRKDVSLTCLVVGENPGDISVEWTSNGHTEENYKDTAPVLDSDGSYFI |
| | linker in | YSKLNMKTSKWEKTDSFSCNVRHEGLKNYYLKKTISRSPGK 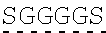 APTSSSTKKTQLQ |
| | dashed | LEHLLLDLQMILNGINNYKNPKLTRMLTKKFrMPKKATELKHLQCLEEELKPLEEr |
| | underline | LNLAQSKNFHLRPRDLISNINVIVLELKGSETTEMCEYADETATIVEFLNRWITFCQSIISTLT |
| | hIL-2 in | |
| | italics | |
| 570 | anti-KLH-C3- | EVQLVGSGGGLVQPGGSLKLSCAASGFTFSDFYMAWVRQAPTKGLEWVASISTGGGNTHYRDSVKGRFT |
| | mIgG2b-N297A- | ISRDNAKSTLYLQMDSLRSEETATYYCARLLSTISTPFDYWGQGVIVTVSSAKTTPPSVYPLAPGCGDT |
| | L6-hIL-2 | TGSSVTLGCLVKGYFPESVTVTWNSGSLSSSVHTFPALLQSGLYTMSSSVTVPSSTWPSQTVTCSVAHP |
| | (F42K/Y45R/V69R) | ASSTTVDKKLEPSGPISTINPCPPCKECHKCPAPNLEGGPSVFIFPPNIKDVLMISLTPKVTCVVVDVS |
| | HC | EDDPDVQISWFVNNVEVHTAQTQTHREDYASTIRVVSTLPIQHQDWMSGKEFKCKVNNKDLPSPIERTI |
| | linker in | SKIKGLVRAPQVYILPPPAEQLSRKDVSLTCLVVGENPGDISVEWTSNGHTEENYKDTAPVLDSDGSYF |
| | dashed | IYSKLNMKTSKWEKTDSFSCNVRHEGLKNYYLKKTISRSPGK 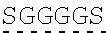 APTSSSTK |
| | underline | KTQLQLEHLLLDLQMILNGINNYKNPKLTRMLTKKFrMPKKATELKHLQCLEEELKPLEErLNLA |
| | hIL-2 in | QSKNFHLRPRDLISNINVIVLELKGSETTEMCEYADETATIVEFLNRWITFCOSIISTLT |
| | italics | |
| 571 | KLH-C3-mKappa | DVVLIQSPTTLSVTPGETVSLSCRASHSVGTNLHWYQQRTNESPSLLIKYSSHSTSGIPSRESATGSGT |
| | LC | DFTLNISNVEFDDVASYFCQQSQKWPLTFGSGTKLEIKRADAAPTVSIFPPSSEQLTSGGASVVCFINN |
| | | FYPKDINVKWKIDGSERQNGVLNSWTDQDSKDSTYSMSSTLTLTKDEYERHNSYTCEATHKTSTSPIVK |
| | | SENRNEC |
| 572 | 2D12-hIgG1-L6- | EVQLQQSGPELVKPGASVKISCKTSGYTFTEYTMHWVKQSHGKSLEWIGGINPNNGGTTYNQKFKGKAT |
| | hIL-2 HC | LTVDKSSSTAYMELRSLTSQDSAVYYCARDYYRYGHYYAMDYWGQGTSVTVSSASTKGPSVFPLAPSSK |
| | linker in | STSGGTAALGCLVKDYFPEPVTVSWNSGALTSGVHTFPAVLQSSGLYSLSSVVTVPSSSLGTQTYICNV |
| | dashed | NHKPSNTKVDKKVEPKSCDKTHTCPPCPAPELLGGPSVFLFPPKPKDTLMISRTPEVTCVVVDVSHEDP |
| | underline | EVKFNWYVDGVEVHNAKTKPREEQYNSTYRVVSVLTVLHQDWLNGKEYKCKVSNKALPAPIEKTISKAK |
| | hIL-2 in | GQPREPQVYTLPPSRDELTKNQVSLTCLVKGFYPSDIAVEWESNGQPENNYKTTPPVLDSDGSFFLYSK |
| | italics | LTVDKSRWQQGNVFSCSVMHEALHNHYTQKSLSLSPGK 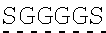 APTSSSTKKTQ |
| | | LQLEHLLLDLQMILNGINNYKNPKLTRMLTFKFYMPKKATELKHLQCLEEELKPLEEVLNL |
| | | AQSKNFHLRPRDLISNINVIVLELKGSETTFMCEYADETATIVEFLNRWITFCQSIISTLT |
| 573 | 2D12-hKappa LC | QIVLTQSPAIMSASPGEKVTMTCSVSSSVREMHWYQQKSGTSPKRWIYDTSKLASGVPARFSGSGSGTS |
| | | YSLTISSMEAEDAATYYCQQWSSNPPTFGGGTKLKIKRTVAAPSVFIFPPSDEQLKSGTASVVCLLNNE |
| | | YPREAKVQWKVDNALQSGNSQESVTEQDSKDSTYSLSSTLTLSKADYEKHKVYACEVTHQGLSSPVTKS |
| | | ENRGEC |
| 574 | hIL-2 | APTSSSTKKTQLQLEHLLLDLQMILNGINNYKNPKLTRMLTAKFAMPKKATELKHLQCLEEELKPLEEV |
| | F42A/Y45A/L72G | LNGAQSKNFHLRPRDLISNINVIVLELKGSETTFMCEYADETATIVEFLNRWITFCQSIISTLT |
| 575 | hIL-2 | APTSSSTKKTQLQLEALLLDLQMILNGINNYKNPKLTRMLTAKFYMPKKATELKHLQCLEEELKPLEEV |
| | H16A/F42A | LNLAQSKNFHLRPRDLISNINVIVLELKGSETTFMCEYADETATIVEFLNRWITFCQSIISTLT |
| 576 | 1H9-hIgG4 HC | EVQLLESGGGLVQPGGSLRLSCVGSGFNLKDYCMTWVRQAPGKGLEWVSAIVYSGGSTYYADSVKGRFT |
| | | ISRDNSKNTLYLQMNSLRAEDTAVYYCAKYTRGSYFYDAMDVWGQGTTVTVSSASTKGPSVFPLAPCSR |
| | | STSESTAALGCLVKDYFPEPVTVSWNSGALTSGVHTFPAVLQSSGLYSLSSVVTVPSSSLGTKTYTCNV |
| | | DHKPSNTKVDKRVESKYGPPCPPCPAPEFLGGPSVFLFPPKPKDTLMISRTPEVTCVVVDVSQEDPEVQ |
| | | FNWYVDGVEVHNAKTKPREEQENSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLG |
| 577 | 1D5-hIgG4 HC | EVQLLESGGGLVQPGGSLRLSCVGSGENFKDYCMTWVRQAPGKGLEWVSAIVYSGGSTYYADSVKGRFT |
| | | ISRDNSKNTLYLQMNSLRAEDTAVYYCAKYTRGSYFYDAMDVWGQGTTVTVSSASTKGPSVFPLAPCSR |
| | | STSESTAALGCLVKDYFPEPVTVSWNSGALTSGVHTFPAVLOSSGLYSLSSVVTVPSSSLGTKTYTCNV |
| | | DHKPSNTKVDKRVESKYGPPCPPCPAPEFLGGPSVELFPPKPKDTLMISRTPEVTCVVVDVSQEDPEVQ |
| | | FNWYVDGVEVHNAKTKPREEQFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNYKTTPPVLDSDGSFFLYSRLTV |
| | | DKSRWQEGNVFSCSVMHEALHNHYTQKSLSLSLG |
| 578 | Anti-hPD-1 #2 | QVQLVQSGVEVKKPGASVKVSCKASGYTFTNYYMYWVRQAPGQGLEWMGGINPSNGGTNENEKEKNRVT |
| | HC | LTTDSSTTTAYMELKSLQFDDTAVYYCARRDYRFDMGEDYWGQGTTVTVSSASTKGPSVFPLAPCSRST |
| | | SESTAALGCLVKDYFPEPVTVSWNSGALTSGVHTFPAVLQSSGLYSLSSVVTVPSSSLGTKTYTCNVDH |
| | | KPSNTKVDKRVESKYGPPCPPCPAPEFLGGPSVELFPPKPKDTLMISRTPEVTCVVVDVSQEDPEVQEN |
| | | WYVDGVEVHNAKTKPREEQFNSTYRVVSVLTVLHQDWLNGKEYKCKVSNKGLPSSIEKTISKAKGQPRE |
| | | PQVYTLPPSQEEMTKNQVSLTCLVKGFYPSDIAVEWESNGQPENNYKTTPPVLDSDGSFFLYSRLTVDK |
| | | SRWQEGNVFSCSVMHEALHNHYTQKSLSLSLG |
| 579 | Anti-hPD-1 #2 | EIVLTQSPATLSLSPGERATLSCRASKGVSTSGYSYLHWYQQKPGQAPRLLIYLASYLESGVPARESGS |
| | LC | GSGTDFTLTISSLEPEDFAVYYCQHSRDLPLTFGGGTKVEIKRTVAAPSVFIFPPSDEQLKSGTASVVC |
| | | LLNNFYPREAKVQWKVDNALQSGNSQESVTEQDSKDSTYSLSSTLTLSKADYEKHKVYACEVTHQGLSS |
| | | PVTKSENRGEC |
| 580 | human PD-1 | gtcgacggatcgggagatctcccgatcccctatggtgcactctcagtacaatctgctctgatgccgcat |
| | receptor | agttaagccagtatctgctccctgcttgtgtgttggaggtcgctgagtagtgcgcgagcaaaatttaag |
| | lentiviral | ctacaacaaggcaaggcttgaccgacaattgcatgaagaatctgcttagggttaggcgttttgcgctgc |
| | vector | ttcgcgatgtacgggccagatatacgcgttgacattgattattgactagttattaatagtaatcaatta |
| | | cggggtcattagttcatagcccatatatggagttccgcgttacataacttacggtaaatggcccgcctg |
| | | gctgaccgcccaacgacccccgcccattgacgtcaataatgacgtatgttcccatagtaacgccaatag |
| | | ggactttccattgacgtcaatgggtggagtatttacggtaaactgcccacttggcagtacatcaagtgt |
| | | atcatatgccaagtacgccccctattgacgtcaatgacggtaaatggcccgcctggcattatgcccagt |
| | | acatgaccttatgggactttcctacttggcagtacatctacgtattagtcatcgctattaccatggtga |
| | | tgcggttttggcagtacatcaatgggcgtggatagcggtttgactcacggggatttccaagtctccacc |
| | | ccattgacgtcaatgggagtttgttttggcaccaaaatcaacgggactttccaaaatgtcgtaacaact |
| | | ccgccccattgacgcaaatgggcggtaggcgtgtacggtgggaggtctatataagcagcgcgttttgcc |
| | | tgtactgggtctctctggttagaccagatctgagcctgggagctctctggctaactagggaacccactg |
| | | cttaagcctcaataaagcttgccttgagtgcttcaagtagtgtgtgcccgtctgttgtgtgactctggt |
| | | aactagagatccctcagacccttttagtcagtgtggaaaatctctagcagtggcgcccgaacagggact |
| | | tgaaagcgaaagggaaaccagaggagctctctcgacgcaggactcggcttgctgaagcgcgcacggcaa |
| | | gaggcgaggggcggcgactggtgagtacgccaaaaattttgactagcggaggctagaaggagagagatg |
| | | ggtgcgagagcgtcagtattaagcgggggagaattagatcgcgatgggaaaaaattcggttaaggccag |
| | | ggggaaagaaaaaatataaattaaaacatatagtatgggcaagcagggagctagaacgattcgcagtta |
| | | atcctggcctgttagaaacatcagaaggctgtagacaaatactgggacagctacaaccatcccttcaga |
| | | caggatcagaagaacttagatcattatataatacagtagcaaccctctattgtgtgcatcaaaggatag |
| | | agataaaagacaccaaggaagctttagacaagatagaggaagagcaaaaaaaagtaagaccaccgcac |
| | | agcaagcggccgctgatcttcagacctggaggaggagatatgagggacaattggagaagtgaattatat |
| | | aaatataaagtagtaaaaattgaaccattaggagtagcacccaccaaggcaaagagaagagtggtgcag |
| | | agagaaaaaagagcagtgggaataggagctttgttccttgggttcttgggagcagcaggaagcactatg |
| | | ggcgcagcgtcaatgacgctgacggtacaggccagacaattattgtctggtatagtgcagcagcagaac |
| | | aatttgctgagggctattgaggcgcaacagcatctgttgcaactcacagtctggggcatcaagcagctc |
| | | caggcaagaatcctggctgtggaaagatacctaaaggatcaacagctcctggggatttggggttgctct |
| | | ggaaaactcatttgcaccactgctgtgccttggaatgctagttggagtaataaatctctggaacagatt |
| | | tggaatcacacgacctggatggagtgggacagagaaattaacaattacacaagcttaatacactcctta |
| | | attgaagaatcgcaaaaccagcaagaaaagaatgaacaagaattattggaattagataaatgggcaagt |
| | | ttgtggaattggtttaacataacaaattggctgtggtatataaaattattcataatgatagtaggaggc |
| | | ttggtaggtttaagaatagtttttgctgtactttctatagtgaatagagttaggcagggatattcacca |
| | | ttatcgtttcagacccacctcccaaccccgaggggacccgacaggcccgaaggaatagaagaagaaggt |
| | | ggagagagagacagagacagatccattcgattagtgaacggatcggcactgcgtgcgccaattctgcag |
| | | acaaatggcagtattcatccacaattttaaaagaaaaggggggattggggggtacagtgcaggggaaag |
| | | aatagtagacataatagcaacagacatacaaactaaagaattacaaaaacaaattacaaaaattcaaaa |
| | | ttttcgggtttattacagggacagcagagatccagtttggttaattaagtaattcgctagctaggtctt |
| | | gaaaggagtgggaattggctccggtgcccgtcagtgggcagagcgcacatcgcccacagtccccgagaa |
| | | gttggggggaggggtcggcaattgatccggtgcctagagaaggtggcgcggggtaaactgggaaagtga |
| | | tgtcgtgtactggctccgcctttttcccgagggtgggggagaaccgtatataagtgcagtagtcgccgt |
| | | gaacgttctttttcgcaacgggtttgccgccagaacacaggaccggttctagagcgctgccaccatgca |
| | | gatcccacaggcgccctggccagtcgtctgggcggtgctacaactgggctggcggccaggatggttctt |
| | | agactccccagacaggccctggaacccccccaccttctccccagccctgctcgtggtgaccgaagggga |
| | | caacgccaccttcacctgcagcttctccaacacatcggagagcttcgtgctaaactggtaccgcatgag |
| | | ccccagcaaccagacggacaagctggccgccttccccgaggaccgcagccagcccggccaggactgccg |
| | | cttccgtgtcacacaactgcccaacgggcgtgacttccacatgagcgtggtcagggcccggcgcaatga |
| | | cagcggcacctacctctgtggggccatctccctggcccccaaggcgcagatcaaagagagcctgcgggc |
| | | agagctcagggtgacagagagaagggcagaagtgcccacagcccaccccagcccctcacccaggccagc |
| | | cggccagttccaaaccctggtggttggtgtcgtgggcggcctgctgggcagcctggtgctgctagtctg |
| | | ggtcctggccgtcatctgctcccgggccgcacgagggacaataggagccaggcgcaccggccagcccct |
| | | gaaggaggacccctcagccgtgcctgtgttctctgtggactatggggagctggatttccagtggcgaga |
| | | gaagaccccggagccccccgtgccctgtgtccctgagcagacggagtatgccaccattgtctttcctag |
| | | cggaatgggcacctcatcccccgcccgcaggggctcagctgacggccctcggagtgcccagccactgag |
| | | gcctgaggatggacactgctcttggcccctctgagcccctctccctccccccccctaacgttactggcc |
| | | gaagccgcttggaataaggccggtgtgcgtttgtctatatgttattttccaccatattgccgtcttttg |
| | | gcaatgtgagggcccggaaacctggccctgtcttcttgacgagcattcctaggggtctttcccctctcg |
| | | ccaaaggaatgcaaggtctgttgaatgtcgtgaaggaagcagttcctctggaagcttcttgaagacaaa |
| | | caacgtctgtagcgaccctttgcaggcagcggaaccccccacctggcgacaggtgcctctgcggccaaa |
| | | agccacgtgtataagatacacctgcaaaggcggcacaaccccagtgccacgttgtgagttggatagttg |
| | | tggaaagagtcaaatggctctcctcaagcgtattcaacaaggggctgaaggatgcccagaaggtacccc |
| | | attgtatgggatctgatctggggcctcggtgcacatgctttacatgtgtttagtcgaggttaaaaaaac |
| | | gtctaggccccccgaaccacggggacgtggttttcctttgaaaaacacgatgataatatggccacaatg |
| | | accgagtacaagcccacggtgcgcctcgccacccgcgacgacgtccccagggccgtacgcaccctcgcc |
| | | gccgcgttcgccgactaccccgccacgcgccacaccgtcgatccggaccgccacatcgagcgggtcacc |
| | | gagctgcaagaactcttcctcacgcgcgtcgggctcgacatcggcaaggtgtgggtcgcggacgacggc |
| | | gccgcggtggcggtctggaccacgccggagagcgtcgaagcgggggcggtgttcgccgagatcggcccg |
| | | cgcatggccgagttgagcggttcccggctggccgcgcagcaacagatggaaggcctcctggcgccgcac |
| | | cggcccaaggagcccgcgtggttcctggccaccgtcggagtctcgcccgaccaccagggcaagggtctg |
| | | ggcagcgccgtcgtgctccccggagtggaggcggccgagcgcgccggggtgcccgccttcctggagacc |
| | | tccgcgccccgcaacctccccttctacgagcggctcggcttcaccgtcaccgccgacgtcgaggtgccc |
| | | gaaggaccgcgcacctggtgcatgacccgcaagcccggtgcctgaacgcgttaagtcgacaatcaacct |
| | | ctggattacaaaatttgtgaaagattgactggtattcttaactatgttgctccttttacgctatgtgga |
| | | tacgctgctttaatgcctttgtatcatgctattgcttcccgtatggctttcattttctcctccttgtat |
| | | aaatcctggttgctgtctctttatgaggagttgtggcccgttgtcaggcaacgtggcgtggtgtgcact |
| | | gtgtttgctgacgcaacccccactggttggggcattgccaccacctgtcagctcctttccgggactttc |
| | | gctttccccctccctattgccacggcggaactcatcgccgcctgccttgcccgctgctggacaggggct |
| | | cggctgttgggcactgacaattccgtggtgttgtcggggaaatcatcgtcctttccttggctgctcgcc |
| | | tgtgttgccacctggattctgcgcgggacgtccttctgctacgtcccttcggccctcaatccagcggac |
| | | cttccttcccgcggcctgctgccggctctgcggcctcttccgcgtcttcgccttcgccctcagacgagt |
| | | cggatctccctttgggccgcctccccgcgtcgactttaagaccaatgacttacaaggcagctgtagatc |
| | | ttagccactttttaaaagaaaaggggggactggaagggctaattcactcccaacgaagacaagatctgc |
| | | tttttgcttgtactgggtctctctggttagaccagatctgagcctgggagctctctggctaactaggga |
| | | acccactgcttaagcctcaataaagcttgccttgagtgcttcaagtagtgtgtgcccgtctgttgtgtg |
| | | actctggtaactagagatccctcagacccttttagtcagtgtggaaaatctctagcagggcccgtttaa |
| | | acccgctgatcagcctcgactgtgccttctagttgccagccatctgttgtttgcccctcccccgtgcct |
| | | tccttgaccctggaaggtgccactcccactgtcctttcctaataaaatgaggaaattgcatcgcattgt |
| | | ctgagtaggtgtcattctattctggggggggggtggggcaggacagcaagggggaggattgggaagac |
| | | aatagcaggcatgctggggatgcggtgggctctatggcttctgaggcggaaagaaccagctggggctct |
| | | agggggtatccccacgcgccctgtagcggcgcattaagcgcggcgggtgtggtggttacgcgcagcgtg |
| | | accgctacacttgccagcgccctagcgcccgctcctttcgctttcttcccttcctttctcgccacgttc |
| | | gccggctttccccgtcaagctctaaatcgggggctccctttagggttccgatttagtgctttacggcac |
| | | ctcgaccccaaaaaacttgattagggtgatggttcacgtagtgggccatcgccctgatagacggttttt |
| | | cgccctttgacgttggagtccacgttctttaatagtggactcttgttccaaactggaacaacactcaac |
| | | cctatctcggtctattcttttgatttataagggattttgccgatttcggcctattggttaaaaaatgag |
| | | ctgatttaacaaaaatttaacgcgaattaattctgtggaatgtgtgtcagttagggtgtggaaagtccc |
| | | caggctccccagcaggcagaagtatgcaaagcatgcatctcaattagtcagcaaccaggtgtggaaagt |
| | | ccccaggctccccagcaggcagaagtatgcaaagcatgcatctcaattagtcagcaaccatagtcccgc |
| | | ccctaactccgcccatcccgcccctaactccgcccagttccgcccattctccgccccatggctgactaa |
| | | ttttttttatttatgcagaggccgaggccgcctctgcctctgagctattccagaagtagtgaggaggct |
| | | tttttggaggcctaggcttttgcaaaaagctcccgggagcttgtatatccattttcggatctgatcagc |
| | | acgtgttgacaattaatcatcggcatagtatatcggcatagtataatacgacaaggtgaggaactaaac |
| | | catggccaagttgaccagtgccgttccggtgctcaccgcgcgcgacgtcgccggagcggtcgagttctg |
| | | gaccgaccggctcgggttctcccgggacttcgtggaggacgacttcgccggtgtggtccgggacgacgt |
| | | gaccctgttcatcagcgcggtccaggaccaggtggtgccggacaacaccctggcctgggtgtgggtgcg |
| | | cggcctggacgagctgtacgccgagtggtcggaggtcgtgtccacgaacttccgggacgcctccgggcc |
| | | ggccatgaccgagatcggcgagcagccgtgggggcgggagttcgccctgcgcgacccggccggcaactg |
| | | cgtgcacttcgtggccgaggagcaggactgacacgtgctacgagatttcgattccaccgccgccttcta |
| | | tgaaaggttgggcttcggaatcgttttccgggacgccggctggatgatcctccagcgcggggatctcat |
| | | gctggagttcttcgcccaccccaacttgtttattgcagcttataatggttacaaataaagcaatagcat |
| | | cacaaatttcacaaataaagcatttttttcactgcattctagttgtggtttgtccaaactcatcaatgt |
| | | atcttatcatgtctgtataccgtcgacctctagctagagcttggcgtaatcatggtcatagctgtttcc |
| | | tgtgtgaaattgttatccgctcacaattccacacaacatacgagccggaagcataaagtgtaaagcctg |
| | | gggtgcctaatgagtgagctaactcacattaattgcgttgcgctcactgcccgctttccagtcgggaaa |
| | | cctgtcgtgccagctgcattaatgaatcggccaacgcgcggggagaggcggtttgcgtattgggcgctc |
| | | ttccgcttcctcgctcactgactcgctgcgctcggtcgttcggctgcggcgagcggtatcagctcactc |
| | | aaaggcggtaatacggttatccacagaatcaggggataacgcaggaaagaacatgtgagcaaaaggcca |
| | | gcaaaaggccaggaaccgtaaaaaggccgcgttgctggcgtttttccataggctccgcccccctgacga |
| | | gcatcacaaaaatcgacgctcaagtcagaggtggcgaaacccgacaggactataaagataccaggcgtt |
| | | tccccctggaagctccctcgtgcgctctcctgttccgaccctgccgcttaccggatacctgtccgcctt |
| | | tctcccttcgggaagcgtggcgctttctcatagctcacgctgtaggtatctcagttcggtgtaggtcgt |
| | | tcgctccaagctgggctgtgtgcacgaaccccccgttcagcccgaccgctgcgccttatccggtaacta |
| | | tcgtcttgagtccaacccggtaagacacgacttatcgccactggcagcagccactggtaacaggattag |
| | | cagagcgaggtatgtaggcggtgctacagagttcttgaagtggtggcctaactacggctacactagaag |
| | | aacagtatttggtatctgcgctctgctgaagccagttaccttcggaaaaagagttggtagctcttgatc |
| | | cggcaaacaaaccaccgctggtagcggtggtttttttgtttgcaagcagcagattacgcgcagaaaaaa |
| | | aggatctcaagaagatcctttgatcttttctacggggtctgacgctcagtggaacgaaaactcacgtta |
| | | agggattttggtcatgagattatcaaaaaggatcttcacctagatccttttaaattaaaaatgaagttt |
| | | taaatcaatctaaagtatatatgagtaaacttggtctgacagttaccaatgcttaatcagtgaggcacc |
| | | tatctcagcgatctgtctatttcgttcatccatagttgcctgactccccgtcgtgtagataactacgat |
| | | acgggagggcttaccatctggccccagtgctgcaatgataccgcgagacccacgctcaccggctccaga |
| | | tttatcagcaataaaccagccagccggaagggccgagcgcagaagtggtcctgcaactttatccgcctc |
| | | catccagtctattaattgttgccgggaagctagagtaagtagttcgccagttaatagtttgcgcaacgt |
| | | tgttgccattgctacaggcatcgtggtgtcacgctcgtcgtttggtatggcttcattcagctccggttc |
| | | ccaacgatcaaggcgagttacatgatcccccatgttgtgcaaaaaagcggttagctccttcggtcctcc |
| | | gatcgttgtcagaagtaagttggccgcagtgttatcactcatggttatggcagcactgcataattctct |
| | | tactgtcatgccatccgtaagatgcttttctgtgactggtgagtactcaaccaagtcattctgagaata |
| | | gtgtatgcggcgaccgagttgctcttgcccggcgtcaatacgggataataccgcgccacatagcagaac |
| | | tttaaaagtgctcatcattggaaaacgttcttcggggcgaaaactctcaaggatcttaccgctgttgag |
| | | atccagttcgatgtaacccactcgtgcacccaactgatcttcagcatcttttactttcaccagcgtttc |
| | | tgggtgagcaaaaacaggaaggcaaaatgccgcaaaaaagggaataagggcgacacggaaatgttgaat |
| | | actcatactcttcctttttcaatattattgaagcatttatcagggttattgtctcatgagcggatacat |
| | | atttgaatgtatttagaaaaataaacaaataggggttccgcgcacatttccccgaaaagtgccacctga |
| | | C |
| 581 | hIL-2 V69R | APTSSSTKKTQLQLEHLLLDLQMILNGINNYKNPKLTRMLTFKFYMPKKATELKHLQCLEEELKPLEER |
| | | LNLAQSKNFHLRPRDLISNINVIVLELKGSETTFMCEYADETATIVEFLNRWITFCQSIISTLT |
| 582 | H7-02-hIgG1- | EVQLLESGGGLVQPGGSLRLSCAASGFTFKSYAMTWVRQAPGKGLEWVSAIVYSGGSTYYADSVKGRFT |
| | LAGA-df-hIL-2 | ISRDNSKNTLYLQMNSLRAEDTAVYYCAKYTRASYFYDAMDVWGQGTTVTVSSASTKGPSVFPLAPSSK |
| | (T3A/D20A/R38E/ | STSGGTAALGCLVKDYFPEPVTVSWNSGALTSGVHTFPAVLQSSGLYSLSSVVTVPSSSLGTQTYICNV |
| | C125A) HC | NHKPSNTKVDKKVEPKSCDKTHTCPPCPAPELAGAPSVELFPPKPKDTLMISRTPEVTCVVVDVSHEDP |
| | | EVKFNWYVDGVEVHNAKTKPREEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENNYKTTPPVLDSDGSFFLYSK |
| | | LTVDKSRWQQGNVFSCSVMHEALHNHYTQKSLSLSPGKAPASSSTKKTQLQLEHLLLALQMILNGINNY |
| | | KNPKLTEMLTFKFYMPKKATELKHLQCLEEELKPLEEVLNLAQSKNFHLRPRDLISNINVIVLELKGSE |
| | | TTFMCEYADETATIVEFLNRWITFAQSIISTLT |
| 583 | H7-02-hKappa | EIVLTQSPGTLSLSPGERATLSCRASQSISSSFLAWYQQKPGQAPRLLIYDASTRATGIPDRESGSGSG |
| | LC | TDFTLTISRLEPEDFAVYYCQQYYDWPPLSFGGGTKVEIKRTVAAPSVFIFPPSDEQLKSGTASVVCLL |
| | | NNFYPREAKVQWKVDNALQSGNSQESVTEQDSKDSTYSLSSTLTLSKADYEKHKVYACEVTHQGLSSPV |
| | | TKSENRGEC |
| 584 | hPD-L1 | MRIFAVFIFMTYWHLLNAFTVTVPKDLYVVEYGSNMTIECKFPVEKQLDLAALIVYWEMEDKNIIQFVH |
| | | GEEDLKVQHSSYRQRARLLKDQLSLGNAALQITDVKLQDAGVYRCMISYGGADYKRITVKVNAPYNKIN |
| | | QRILVVDPVTSEHELTCQAEGYPKAEVIWTSSDHQVLSGKTTTTNSKREEKLFNVTSTLRINTTTNEIF |
| | | YCTFRRLDPEENHTAELVIPELPLAHPPNERTHLVILGAILLCLGVALTFIFRLRKGRMMDVKKCGIQD |
| | | TNSKKQSDTHLEET |
| 585 | KLH-C3-hIgG4 | EVQLVGSGGGLVQPGGSLKLSCAASGFTFSDFYMAWVRQAPTKGLEWVASISTGGGNTHYRDSVKGRET |
| | HC | ISRDNAKSTLYLQMDSLRSEETATYYCARLLSTISTPEDYWGQGVIVTVSSASTKGPSVEPLAPCSRST |
| | | SESTAALGCLVKDYFPEPVTVSWNSGALTSGVHTFPAVLQSSGLYSLSSVVTVPSSSLGTKTYTCNVDH |
| | | KPSNTKVDKRVESKYGPPCPPCPAPEFLGGPSVELFPPKPKDTLMISRTPEVTCVVVDVSQEDPEVQEN |
| | | WYVDGVEVHNAKTKPREEQFNSTYRVVSVLTVLHQDWLNGKEYKCKVSNKGLPSSIEKTISKAKGQPRE |
| | | PQVYTLPPSQEEMTKNQVSLTCLVKGFYPSDIAVEWESNGQPENNYKTTPPVLDSDGSFFLYSRLTVDK |
| | | SRWQEGNVFSCSVMHEALHNHYTQKSLSLSLGK |
| 586 | KLH-C3-hKappa | DVVLIQSPTTLSVTPGETVSLSCRASHSVGTNLHWYQQRTNESPSLLIKYSSHSTSGIPSRFSATGSGT |
| | LC | DFTLNISNVEFDDVASYFCQQSQKWPLTFGSGTKLEIKRTVAAPSVFIFPPSDEQLKSGTASVVCLINN |
| | | FYPREAKVQWKVDNALQSGNSQESVTEQDSKDSTYSLSSTLTLSKADYEKHKVYACEVTHQGLSSPVTK |
| | | SFNRGEC |
| 587 | 1H3-hIgG1-L6- | EVOLVESGGGLVQPGRSLKLSCAVSGFTESDYAMAWVRQAPKKGLEWVATISYDGSRTYYRDSVKGRET |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (E15A) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMOSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLAHLLLDLQMILNGINNYKNPKLTRMLTFKFYMPKKATEL |
| | | KHLQCLEEELKPLEEVLNLAQSKNFHLRPRDLISNINVIVLELKGSETTFMCEYADETATIVEFLNRWI |
| | | TFCQSIISTLT |
| 588 | 1H3-hIgG1-L6- | EVQLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLOMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (D20I) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLILQMILNGINNYKNPKLTRMLTFKFYMPKKATEL |
| | | KHLQCLEEELKPLEEVLNLAQSKNFHLRPRDLISNINVIVLELKGSETTFMCEYADETATIVEFLNRWI |
| | | TFCQSIISTLT |
| 589 | 1H3-hIgG1-L6- | EVQLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (D20S) HC | NTKVDKKVEPKSCDKTHTCPPCPAPELLGGPSVELFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLSLQMILNGINNYKNPKLTRMLTFKFYMPKKATEL |
| | | KHLQCLEEELKPLEEVLNLAQSKNFHLRPRDLISNINVIVLELKGSETTEMCEYADETATIVEFLNRWI |
| | | TFCQSIISTLT |
| 590 | 1H3-hIgG1-L6- | EVQLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (D20H) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLHLQMILNGINNYKNPKLTRMLTFKFYMPKKATEL |
| | | KHLQCLEEELKPLEEVLNLAQSKNFHLRPRDLISNINVIVLELKGSETTFMCEYADETATIVEFLNRWI |
| | | TFCQSIISTLT |
| 591 | 1H3-hIgG1-L6- | EVQLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (D20W) HC | NTKVDKKVEPKSCDKTHTCPPCPAPELLGGPSVELFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLWLQMILNGINNYKNPKLTRMLTFKFYMPKKATEL |
| | | KHLQCLEEELKPLEEVLNLAQSKNFHLRPRDLISNINVIVLELKGSETTFMCEYADETATIVEFLNRWI |
| | | TFCQSIISTLT |
| 592 | 1H3-hIgG1-L6- | EVOLVESGGGLVQPGRSLKLSCAVSGFTFSDYAMAWVRQAPKKGLEWVATISYDGSRTYYRDSVKGRET |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (D20Y) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLYLQMILNGINNYKNPKLTRMLTFKFYMPKKATEL |
| | | KHLQCLEEELKPLEEVLNLAQSKNFHLRPRDLISNINVIVLELKGSETTEMCEYADETATIVEFLNRWI |
| | | TFCQSIISTLT |
| 593 | 1H3-hIgG1-L6- | EVQLVESGGGLVQPGRSLKLSCAVSGFTESDYAMAWVRQAPKKGLEWVATISYDGSRTYYRDSVKGRFT |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (D20R) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLRLQMILNGINNYKNPKLTRMLTFKFYMPKKATEL |
| | | KHLQCLEEELKPLEEVLNLAQSKNFHLRPRDLISNINVIVLELKGSETTFMCEYADETATIVEFLNRWI |
| | | TFCQSIISTLT |
| 594 | 1H3-hIgG1-L6- | EVQLVESGGGLVQPGRSLKLSCAVSGFTFSDYAMAWVRQAPKKGLEWVATISYDGSRTYYRDSVKGRET |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (D20F) HC | NTKVDKKVEPKSCDKTHTCPPCPAPELLGGPSVELFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLFLQMILNGINNYKNPKLTRMLTFKFYMPKKATEL |
| | | KHLQCLEEELKPLEEVLNLAQSKNFHLRPRDLISNINVIVLELKGSETTFMCEYADETATIVEFLNRWI |
| | | TFCQSIISTLT |
| 595 | 1H3-hIgG1-L6- | EVQLVESGGGLVQPGRSLKLSCAVSGFTESDYAMAWVRQAPKKGLEWVATISYDGSRTYYRDSVKGRET |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (D84K) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRKLISNINVIVLELKGSETTFMCEYADETATIVEFLNRWI |
| | | TFCQSIISTLT |
| 596 | 1H3-hIgG1-L6- | EVQLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (S87A) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRDLIANINVIVLELKGSETTFMCEYADETATIVEFLNRWI |
| | | TFCQSIISTLT |
| 597 | 1H3-hIgG1-L6- | EVQLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLOMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (N88Y) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRDLISYINVIVLELKGSETTFMCEYADETATIVEFLNRWI |
| | | TFCQSIISTLT |
| 598 | 1H3-hIgG1-L6- | EVQLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (N88D) HC | NTKVDKKVEPKSCDKTHTCPPCPAPELLGGPSVELFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRDLISDINVIVLELKGSETTFMCEYADETATIVEFLNRWI |
| | | TFCQSIISTLT |
| 599 | 1H3-hIgG1-L6- | EVQLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (N88R) HC | NTKVDKKVEPKSCDKTHTCPPCPAPELLGGPSVELFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRDLISRINVIVLELKGSETTFMCEYADETATIVEFLNRWI |
| | | TFCQSIISTLT |
| 600 | 1H3-hIgG1-L6- | EVOLVESGGGLVQPGRSLKLSCAVSGFTFSDYAMAWVRQAPKKGLEWVATISYDGSRTYYRDSVKGRET |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (N88E) HC | NTKVDKKVEPKSCDKTHTCPPCPAPELLGGPSVELFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRDLISEINVIVLELKGSETTEMCEYADETATIVEFLNRWI |
| | | TFCQSIISTLT |
| 601 | 1H3-hIgG1-L6- | EVOLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (N88F) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRDLISFINVIVLELKGSETTEMCEYADETATIVEFLNRWI |
| | | TFCQSIISTLT |
| 602 | 1H3-hIgG1-L6- | EVQLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (N88I) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRDLISIINVIVLELKGSETTEMCEYADETATIVEFLNRWI |
| | | TFCQSIISTLT |
| 603 | 1H3-hIgG1-L6- | EVOLVESGGGLVQPGRSLKLSCAVSGFTFSDYAMAWVRQAPKKGLEWVATISYDGSRTYYRDSVKGRFT |
| | hCD25 (1-164) - | ISRDNAKITLYLQMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (I92A) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMQSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRDLISNINVAVLELKGSETTEMCEYADETATIVEFLNRWI |
| | | TFCQSIISTLT |
| 604 | 1H3-hIgG1-L6- | EVOLVESGGGLVQPGRSLKLSCAVSGFTESDYAMAWVRQAPKKGLEWVATISYDGSRTYYRDSVKGRFT |
| | hCD25 (1-164) - | ISRDNAKITLYLOMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (E95A) HC | NTKVDKKVEPKSCDKTHTCPPCPAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGOPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMOSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRDLISNINVIVLALKGSETTEMCEYADETATIVEFLNRWI |
| | | TFCQSIISTLT |
| 605 | 1H3-hIgG1-L6- | EVOLVESGGGLVQPGRSLKLSCAVSGFTFSDYAMAWVRQAPKKGLEWVATISYDGSRTYYRDSVKGRET |
| | hCD25 (1-164) - | ISRDNAKITLYLOMDSLRSEDTATYYCARHGSGYFDYWGQGVMVTVSSASTKGPSVFPLAPSSKSTSGG |
| | L20-hIL-2 | TAALGCLVKDYFPEPVTVSWNSGALTSGVHTFPAVLQSSGLYSLSSVVTVPSSSLGTQTYICNVNHKPS |
| | (E95K) HC | NTKVDKKVEPKSCDKTHTCPPCPAPELLGGPSVELFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSFFLYSKLTVDK |
| | | SRWQQGNVFSCSVMHEALHNHYTQKSLSLSPGKSGGGGSELCDDDPPEIPHATFKAMAYKEGTMLNCEC |
| | | KRGFRRIKSGSLYMLCTGNSSHSSWDNQCQCTSSATRNTTKQVTPQPEEQKERKTTEMOSPMQPVDQAS |
| | | LPGHCREPPPWENEATERIYHFVVGQMVYYQCVQGYRALHRGPAESVCKMTHGKTRWTQPQLICTSGGG |
| | | GSGGGGSGGGGSGGGSAPTSSSTKKTQLQLEHLLLDLQMILNGINNYKNPKLTRMLTFKFYMPKKATEL |
| | | KHLQCLEEELKPLEEVLNLAQSKNFHLRPRDLISNINVIVLKLKGSETTEMCEYADETATIVEFLNRWI |
| | | TFCQSIISTLT |
| 606 | hIL-2D20R/ | APTSSSTKKTQLQLEHLLLRLQMILNGINNYKNPKLTEMLTFKFYMPKKATELKHLQCLEEELKPLEEV |
| | R38E | LNLAQSKNFHLRPRDLISNINVIVLELKGSETTEMCEYADETATIVEFLNRWITFCQSIISTLT |
| 607 | hIL-2D20N/ | APTSSSTKKTQLQLEHLLLNLQMILNGINNYKNPKLTEMLTFKFYMPKKATELKHLQCLEEELKPLEEV |
| | R38E | LNLAQSKNFHLRPRDLISNINVIVLELKGSETTEMCEYADETATIVEFLNRWITFCQSIISTLT |
| 608 | hIL-2D20Q/ | APTSSSTKKTQLQLEHLLLQLQMILNGINNYKNPKLTEMLTFKFYMPKKATELKHLOCLEEELKPLEEV |
| | R38E | LNLAQSKNFHLRPRDLISNINVIVLELKGSETTFMCEYADETATIVEFLNRWITFCQSIISTLT |
| 609 | hIL-2D20E/ | APTSSSTKKTQLQLEHLLLELQMILNGINNYKNPKLTEMLTFKFYMPKKATELKHLQCLEEELKPLEEV |
| | R38E | LNLAQSKNFHLRPRDLISNINVIVLELKGSETTEMCEYADETATIVEFLNRWITFCQSIISTLT |
| 610 | hIL-2D20G/ | APTSSSTKKTQLQLEHLLLGLQMILNGINNYKNPKLTEMLTFKFYMPKKATELKHLQCLEEELKPLEEV |
| | R38E | LNLAQSKNFHLRPRDLISNINVIVLELKGSETTEMCEYADETATIVEFLNRWITFCQSIISTLT |
| 611 | hIL-2D20I/ | APTSSSTKKTQLQLEHLLLILQMILNGINNYKNPKLTEMLTFKFYMPKKATELKHLQCLEEELKPLEEV |
| | R38E | LNLAQSKNFHLRPRDLISNINVIVLELKGSETTFMCEYADETATIVEFLNRWITFCQSIISTLT |
| 612 | hIL-2D20L/ | APTSSSTKKTQLQLEHLLLLLQMILNGINNYKNPKLTEMLTFKFYMPKKATELKHLQCLEEELKPLEEV |
| | R38E | LNLAQSKNFHLRPRDLISNINVIVLELKGSETTEMCEYADETATIVEFLNRWITFCQSIISTLT |
| 613 | hIL-2D20K/ | APTSSSTKKTQLQLEHLLLKLQMILNGINNYKNPKLTEMLTFKFYMPKKATELKHLQCLEEELKPLEEV |
| | R38E | LNLAQSKNFHLRPRDLISNINVIVLELKGSETTFMCEYADETATIVEFLNRWITFCQSIISTLT |
| 614 | hIL-2D20M/ | APTSSSTKKTQLQLEHLLLMLQMILNGINNYKNPKLTEMLTFKFYMPKKATELKHLQCLEEELKPLEEV |
| | R38E | LNLAQSKNFHLRPRDLISNINVIVLELKGSETTFMCEYADETATIVEFLNRWITFCQSIISTLT |
| 615 | hIL-2D20F/ | APTSSSTKKTQLQLEHLLLFLQMILNGINNYKNPKLTEMLTFKFYMPKKATELKHLQCLEEELKPLEEV |
| | R38E | LNLAQSKNFHLRPRDLISNINVIVLELKGSETTEMCEYADETATIVEFLNRWITFCQSIISTLT |
| 616 | hIL-2D20P/ | APTSSSTKKTQLQLEHLLLPLQMILNGINNYKNPKLTEMLTFKFYMPKKATELKHLQCLEEELKPLEEV |
| | R38E | LNLAQSKNFHLRPRDLISNINVIVLELKGSETTFMCEYADETATIVEFLNRWITFCQSIISTLT |
| 617 | hIL-2D20T/ | APTSSSTKKTQLQLEHLLLTLQMILNGINNYKNPKLTEMLTFKFYMPKKATELKHLQCLEEELKPLEEV |
| | R38E | LNLAQSKNFHLRPRDLISNINVIVLELKGSETTEMCEYADETATIVEFLNRWITFCQSIISTLT |
| 618 | hIL-2D20W/ | APTSSSTKKTQLQLEHLLLWLQMILNGINNYKNPKLTEMLTFKFYMPKKATELKHLQCLEEELKPLEEV |
| | R38E | LNLAQSKNFHLRPRDLISNINVIVLELKGSETTFMCEYADETATIVEFLNRWITFCQSIISTLT |
| 619 | hIL-2D20Y/ | APTSSSTKKTQLQLEHLLLYLQMILNGINNYKNPKLTEMLTFKFYMPKKATELKHLQCLEEELKPLEEV |
| | R38E | LNLAQSKNFHLRPRDLISNINVIVLELKGSETTFMCEYADETATIVEFLNRWITFCQSIISTLT |
| 620 | hIL-2D20V/ | APTSSSTKKTQLQLEHLLLVLQMILNGINNYKNPKLTEMLTFKFYMPKKATELKHLQCLEEELKPLEEV |
| | R38E | LNLAQSKNFHLRPRDLISNINVIVLELKGSETTEMCEYADETATIVEFLNRWITFCQSIISTLT |
| 621 | hIL-2F42K/ | APTSSSTKKTQLQLEHLLLDLQMILNGINNYKNPKLTRMLTKKFRMPKKATELKHLQCLEEELKPLEER |
| | Y45R/V69R | LNLAQSKNFHLRPRDLISNINVIVLELKGSETTFMCEYADETATIVEFLNRWITFCQSIISTLT |

### Embodiments

The following list of embodiments is intended to complement, rather than displace or supersede, the previous descriptions.

Embodiment 1. A method of treating a cancer in a subject, the method comprising administering to the subject:
(A) an anti-human PD-1 (hPD-1) antibody-modified human interleukin-2 (hIL-2) immunoconjugate comprising:
   a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain complementarity determining region 1 (CDR1) comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411; and
(B) an antagonistic anti-PD-1 antibody that binds PD-1 in the presence of the immunoconjugate.

Embodiment 2. The method of embodiment 1, wherein the antagonistic anti-PD-1 antibody is nivolumab, pembrolizumab, cemiplimab, dostarlimab, or retifanlimab.

Embodiment 3. The method of embodiment 1 or 2, wherein the modified hIL-2 protein comprises the amino acid sequence of any one of SEQ ID NOs: 149, 307, 607-611, 614, 617, or 620.

Embodiment 4. The method of any one of the previous embodiments, wherein the modified hIL-2 protein comprises a D20A substitution relative to the non-modified hIL-2 amino acid sequence of SEQ ID NO: 345 and a R38E substitution relative to the non-modified hIL-2 amino acid sequence of SEQ ID NO: 345.

Embodiment 5. The method of embodiment 4, wherein the modified hIL-2 protein comprises the amino acid sequence of SEQ ID NO: 149.

Embodiment 6. The method of any one of the previous embodiments, wherein the modified hIL-2 protein further comprises a deletion or substitution at amino acid position 3 relative to the non-modified hIL-2 amino acid sequence of SEQ ID NO: 345.

Embodiment 7. The method of embodiment 6, wherein the substitution at amino acid position 3 of the modified hIL-2 protein is T3A.

Embodiment 8. The method of embodiment 7, wherein the modified hIL-2 protein comprises the amino acid sequence of SEQ ID NO: 216.

Embodiment 9. The method of embodiment 6, wherein the modified hIL-2 protein comprises the amino acid sequence of SEQ ID NO: 218.

Embodiment 10. The method of any one of the previous embodiments, wherein the modified hIL-2 protein further comprises a deletion or substitution at amino acid position 125 relative to the non-modified hIL-2 amino acid sequence of SEQ ID NO: 345.

Embodiment 11. The method of embodiment 10, wherein the substitution at amino acid position 125 is C125A.

Embodiment 12. The method of embodiment 11, wherein the modified hIL-2 protein comprises the amino acid sequence of SEQ ID NO: 215, 217, or 219.

Embodiment 13. The method of embodiment 12, wherein the modified hIL-2 protein comprises the amino acid sequence of SEQ ID NO: 217.

Embodiment 14. The method of any one of the previous embodiments, wherein the modified hIL-2 protein is fused to the anti-hPD-1 antibody, or antigen-binding fragment thereof, portion of the immunoconjugate at the N-terminus of an antibody light chain, the C-terminus of an antibody light chain, the N-terminus of an antibody heavy chain, the C-terminus of an antibody heavy chain, the N-terminus of the antigen-binding fragment, or the C-terminus of the antigen-binding fragment.

Embodiment 15. The method of any one of the previous embodiments, wherein the modified hIL-2 protein is directly fused by a peptide bond to the anti-hPD-1 antibody, or antigen-binding fragment thereof, portion of the immunoconjugate.

Embodiment 16. The method of embodiment 15, wherein the modified hIL-2 protein is directly fused by a peptide bond to the C-terminal amino acid residue of the anti-hPD-1 antibody, or antigen-binding fragment thereof, portion of the immunoconjugate.

Embodiment 17. The method of any one of embodiments 1-14, wherein the modified hIL-2 protein is fused to the anti-hPD-1 antibody, or antigen-binding fragment thereof, portion of the immunoconjugate through a linker.

Embodiment 18. The method of any one of the previous embodiments, wherein the anti-hPD-1 antibody, or antigen-binding fragment thereof, portion of the immunoconjugate comprises:
a) a heavy chain variable region comprising the amino acid sequence of SEQ ID NO: 416 and a light chain variable region comprising the amino acid sequence of SEQ ID NO: 417;
b) a heavy chain variable region comprising the amino acid sequence of SEQ ID NO: 384 and a light chain variable region comprising the amino acid sequence of SEQ ID NO: 385;
c) a heavy chain variable region comprising the amino acid sequence of SEQ ID NO: 394 and a light chain variable region comprising the amino acid sequence of SEQ ID NO: 395; or
d) a heavy chain variable region comprising the amino acid sequence of SEQ ID NO: 404 and a light chain variable region comprising the amino acid sequence of SEQ ID NO: 405.

Embodiment 19. The method of any one of the previous embodiments, wherein the anti-hPD-1 antibody, or antigen-binding fragment thereof, portion of the immunoconjugate comprises an IgG1 heavy chain constant region.

Embodiment 20. The method of embodiment 19, wherein the anti-hPD-1 antibody, or antigen-binding fragment thereof, portion of the immunoconjugate comprises an L235A substitution and a G237A substitution, according to EU numbering.

Embodiment 21. The method of any one of the previous embodiments, wherein the anti-hPD-1 antibody, or antigen-binding fragment thereof, portion of the immunoconjugate comprises:
a) a heavy chain comprising the amino acid sequence of SEQ ID NO: 414 and a light chain comprising the amino acid sequence of SEQ ID NO: 415;
b) a heavy chain comprising the amino acid sequence of SEQ ID NO: 424 and a light chain comprising the amino acid sequence of SEQ ID NO: 425;
c) a heavy chain comprising the amino acid sequence of SEQ ID NO: 426 and a light chain comprising the amino acid sequence of SEQ ID NO: 427; or
d) a heavy chain comprising the amino acid sequence of SEQ ID NO: 428 and a light chain comprising the amino acid sequence of SEQ ID NO: 429.

Embodiment 22. The method of embodiment 21, wherein the anti-hPD-1 antibody, or antigen-binding fragment thereof, portion of the immunoconjugate comprises a heavy chain comprising the amino acid sequence of SEQ ID NO: 414 and a light chain comprising the amino acid sequence of SEQ ID NO: 415.

Embodiment 23. The method of any one of the previous embodiments, wherein the immunoconjugate comprises:
a light chain comprising the amino acid sequence of SEQ ID NO: 415; and
a heavy chain-modified hIL-2 protein fusion comprising the amino acid sequence of SEQ ID NO: 532.

Embodiment 24. The method of any one of the previous embodiments, wherein the antagonistic anti-PD-1 antibody and the immunoconjugate are administered to the subject together in a mixture, concurrently as single agents, or sequentially as single agents in any order.

Embodiment 25. The method of embodiment 24, comprising:
administering the immunoconjugate prior to administering the antagonistic anti-PD-1 antibody;
administering the antagonistic anti-PD-1 antibody prior to administering the immunoconjugate; or
administering the immunoconjugate at substantially the same time as administering the antagonistic anti-PD-1 antibody.

Embodiment 26. The method of any one of the previous embodiments, wherein the cancer is melanoma, Merkel cell carcinoma, non-small cell lung carcinoma, renal cell carcinoma, triple negative breast cancer, squamous cell carcinoma of the head/neck, hepatocellular carcinoma, or microsatellite instability-high tumors or tumors with deficient DNA mismatch repair.

Embodiment 27. A method of treating a renal cell carcinoma, a triple negative breast cancer, a squamous cell carcinoma of the head/neck, a Merkel cell carcinoma, a hepatocellular carcinoma, or a microsatellite instability-high tumor or tumor with deficient DNA mismatch repair in a subject, the method comprising administering to the subject:
an anti-human PD-1 (hPD-1) antibody-modified human interleukin-2 (hIL-2) immunoconjugate comprising:
   a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain complementarity determining region 1 (CDR1) comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411.

Embodiment 28. The method of embodiment 27, wherein the modified hIL-2 protein comprises the amino acid sequence of any one of SEQ ID NOs: 149, 307, 607-611, 614, 617, or 620.

Embodiment 29. The method of embodiment 27 or 28, wherein the modified hIL-2 protein comprises a D20A substitution relative to the non-modified hIL-2 amino acid sequence of SEQ ID NO: 345 and a R38E substitution relative to the non-modified hIL-2 amino acid sequence of SEQ ID NO: 345.

Embodiment 30. The method of embodiment 29, wherein the modified hIL-2 protein comprises the amino acid sequence of SEQ ID NO: 149.

Embodiment 31. The method of any one of embodiments 27-30, wherein the modified hIL-2 protein further comprises a deletion or substitution at amino acid position 3 relative to the non-modified hIL-2 amino acid sequence of SEQ ID NO: 345.

Embodiment 32. The method of embodiment 31, wherein the substitution at amino acid position 3 of the modified hIL-2 protein is T3A.

Embodiment 33. The method of embodiment 32, wherein the modified hIL-2 protein comprises the amino acid sequence of SEQ ID NO: 216.

Embodiment 34. The method of embodiment 31, wherein the modified hIL-2 protein comprises the amino acid sequence of SEQ ID NO: 218.

Embodiment 35. The method of any one of embodiments 27-34, wherein the modified hIL-2 protein further comprises a deletion or substitution at amino acid position 125 relative to the non-modified hIL-2 amino acid sequence of SEQ ID NO: 345.

Embodiment 36. The method of embodiment 35, wherein the substitution at amino acid position 125 is C125A.

Embodiment 37. The method of embodiment 36, wherein the modified hIL-2 protein comprises the amino acid sequence of SEQ ID NO: 215, 217, or 219.

Embodiment 38. The method of embodiment 37, wherein the modified hIL-2 protein comprises the amino acid sequence of SEQ ID NO: 217.

Embodiment 39. The method of any one of embodiments 27-38, wherein the modified hIL-2 protein is fused to the anti-hPD-1 antibody, or antigen-binding fragment thereof, portion of the immunoconjugate at the N-terminus of an antibody light chain, the C-terminus of an antibody light chain, the N-terminus of an antibody heavy chain, the C-terminus of an antibody heavy chain, the N-terminus of the antigen-binding fragment, or the C-terminus of the antigen-binding fragment.

Embodiment 40. The method of any one of embodiments 27-39, wherein the modified hIL-2 protein is directly fused by a peptide bond to the anti-hPD-1 antibody, or antigen-binding fragment thereof, portion of the immunoconjugate.

Embodiment 41. The method of embodiment 40, wherein the modified hIL-2 protein is directly fused by a peptide bond to the C-terminal amino acid residue of the anti-hPD-1 antibody, or antigen-binding fragment thereof, portion of the immunoconjugate.

Embodiment 42. The method of any one of embodiments 27-39, wherein the modified hIL-2 protein is fused to the anti-hPD-1 antibody, or antigen-binding fragment thereof, portion of the immunoconjugate through a linker.

Embodiment 43. The method of any one of embodiments 27-42, wherein the anti-hPD-1 antibody, or antigen-binding fragment thereof, portion of the immunoconjugate comprises:
a) a heavy chain variable region comprising the amino acid sequence of SEQ ID NO: 416 and a light chain variable region comprising the amino acid sequence of SEQ ID NO: 417;
b) a heavy chain variable region comprising the amino acid sequence of SEQ ID NO: 384 and a light chain variable region comprising the amino acid sequence of SEQ ID NO: 385;
c) a heavy chain variable region comprising the amino acid sequence of SEQ ID NO: 394 and a light chain variable region comprising the amino acid sequence of SEQ ID NO: 395; or
d) a heavy chain variable region comprising the amino acid sequence of SEQ ID NO: 404 and a light chain variable region comprising the amino acid sequence of SEQ ID NO: 405.

Embodiment 44. The method of any one of embodiments 27-43, wherein the anti-hPD-1 antibody, or antigen-binding fragment thereof, portion of the immunoconjugate comprises an IgG1 heavy chain constant region.

Embodiment 45. The method of embodiment 44, wherein the anti-hPD-1 antibody, or antigen-binding fragment thereof, portion of the immunoconjugate comprises an L235A substitution and a G237A substitution, according to EU numbering.

Embodiment 46. The method of any one of embodiments 27-45, wherein the anti-hPD-1 antibody, or antigen-binding fragment thereof, portion of the immunoconjugate comprises:
a) a heavy chain comprising the amino acid sequence of SEQ ID NO: 414 and a light chain comprising the amino acid sequence of SEQ ID NO: 415;
b) a heavy chain comprising the amino acid sequence of SEQ ID NO: 424 and a light chain comprising the amino acid sequence of SEQ ID NO: 425;
c) a heavy chain comprising the amino acid sequence of SEQ ID NO: 426 and a light chain comprising the amino acid sequence of SEQ ID NO: 427; or
d) a heavy chain comprising the amino acid sequence of SEQ ID NO: 428 and a light chain comprising the amino acid sequence of SEQ ID NO: 429.

Embodiment 47. The method of embodiment 46, wherein the anti-hPD-1 antibody, or antigen-binding fragment thereof, portion of the immunoconjugate comprises a heavy chain comprising the amino acid sequence of SEQ ID NO: 414 and a light chain comprising the amino acid sequence of SEQ ID NO: 415.

Embodiment 48. The method of any one of embodiments 27-47, wherein the immunoconjugate comprises:
a light chain comprising the amino acid sequence of SEQ ID NO: 415; and
a heavy chain-modified hIL-2 protein fusion comprising the amino acid sequence of SEQ ID NO: 532.

Embodiment 49. Use of
(A) an anti-hPD-1 antibody-modified human interleukin-2 (hIL-2) immunoconjugate comprising:
   a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
   an anti-hPD-1 antibody, or antigen-binding fragment thereof, that immunospecifically binds to hPD-1, wherein the antibody or antigen-binding fragment thereof comprises:
      (i) a heavy chain complementarity determining region 1 (CDR1) comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
      (ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
      (iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
      (iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411; and
(B) an antagonistic anti-PD-1 antibody that binds PD-1 in the presence of the immunoconjugate for the treatment of cancer.

What is claimed:

1. A method of treating a cancer in a subject, the method comprising administering to the subject:
(A) an anti-human PD-1 (hPD-1) antibody-modified human interleukin-2 (hIL-2) immunoconjugate comprising:
a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
an anti-hPD-1 antibody, or antigen-binding fragment thereof, that immunospecifically binds to hPD-1, wherein the antibody or antigen-binding fragment thereof comprises:
(i) a heavy chain complementarity determining region 1 (CDR1) comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
(ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
(iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR 1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
(iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411; and
(B) an antagonistic anti-PD-1 antibody that binds PD-1 in the presence of the immunoconjugate.

2. The method of claim 1, wherein the antagonistic anti-PD-1 antibody is nivolumab, pembrolizumab, cemiplimab, dostarlimab, or retifanlimab.

3. The method of claim 1, wherein the modified hIL-2 protein comprises the amino acid sequence of any one of SEQ ID NOs: 149, 307, 607-611, 614, 617, or 620.

4. The method of claim 1, wherein the modified hIL-2 protein comprises a D20A substitution relative to the non-modified hIL-2 amino acid sequence of SEQ ID NO: 345 and a R38E substitution relative to the non-modified hIL-2 amino acid sequence of SEQ ID NO: 345.

5. The method of claim 4, wherein the modified hIL-2 protein comprises the amino acid sequence of SEQ ID NO: 149.

6. The method of claim 1, wherein the modified hIL-2 protein further comprises a deletion or substitution at amino acid position 3 relative to the non-modified hIL-2 amino acid sequence of SEQ ID NO: 345.

7. The method of claim 6, wherein the substitution at amino acid position 3 of the modified hIL-2 protein is T3A.

8. The method of claim 7, wherein the modified hIL-2 protein comprises the amino acid sequence of SEQ ID NO: 216.

9. The method of claim 6, wherein the modified hIL-2 protein comprises the amino acid sequence of SEQ ID NO: 218.

10. The method of claim 1, wherein the modified hIL-2 protein further comprises a deletion or substitution at amino acid position 125 relative to the non-modified hIL-2 amino acid sequence of SEQ ID NO: 345.

11. The method of claim 10, wherein the substitution at amino acid position 125 is C125A.

12. The method of claim 11, wherein the modified hIL-2 protein comprises the amino acid sequence of SEQ ID NO: 215, 217, or 219.

13. The method of claim 12, wherein the modified hIL-2 protein comprises the amino acid sequence of SEQ ID NO: 217.

14. The method of claim 1, wherein the modified hIL-2 protein is fused to the anti-hPD-1 antibody, or antigen-binding fragment thereof, portion of the immunoconjugate at the N-terminus of an antibody light chain, the C-terminus of an antibody light chain, the N-terminus of an antibody heavy chain, the C-terminus of an antibody heavy chain, the N-terminus of the antigen-binding fragment, or the C-terminus of the antigen-binding fragment.

15. The method of claim 1, wherein the modified hIL-2 protein is directly fused by a peptide bond to the anti-hPD-1 antibody, or antigen-binding fragment thereof, portion of the immunoconjugate.

16. The method of claim 15, wherein the modified hIL-2 protein is directly fused by a peptide bond to the C-terminal amino acid residue of the anti-hPD-1 antibody, or antigen-binding fragment thereof, portion of the immunoconjugate.

17. The method of claim 1, wherein the modified hIL-2 protein is fused to the anti-hPD-1 antibody, or antigen-binding fragment thereof, portion of the immunoconjugate through a linker.

18. The method of claim 1, wherein the anti-hPD-1 antibody, or antigen-binding fragment thereof, portion of the immunoconjugate comprises:
a) a heavy chain variable region comprising the amino acid sequence of SEQ ID NO: 416 and a light chain variable region comprising the amino acid sequence of SEQ ID NO: 417;
b) a heavy chain variable region comprising the amino acid sequence of SEQ ID NO: 384 and a light chain variable region comprising the amino acid sequence of SEQ ID NO: 385;
c) a heavy chain variable region comprising the amino acid sequence of SEQ ID NO: 394 and a light chain variable region comprising the amino acid sequence of SEQ ID NO: 395; or
d) a heavy chain variable region comprising the amino acid sequence of SEQ ID NO: 404 and a light chain variable region comprising the amino acid sequence of SEQ ID NO: 405.

19. The method of claim 1, wherein the anti-hPD-1 antibody, or antigen-binding fragment thereof, portion of the immunoconjugate comprises an IgG1 heavy chain constant region.

20. The method of claim 19, wherein the anti-hPD-1 antibody, or antigen-binding fragment thereof, portion of the immunoconjugate comprises an L235A substitution and a G237A substitution, according to EU numbering.

21. The method of claim 1, wherein the anti-hPD-1 antibody, or antigen-binding fragment thereof, portion of the immunoconjugate comprises:
a) a heavy chain comprising the amino acid sequence of SEQ ID NO: 414 and a light chain comprising the amino acid sequence of SEQ ID NO: 415;
b) a heavy chain comprising the amino acid sequence of SEQ ID NO: 424 and a light chain comprising the amino acid sequence of SEQ ID NO: 425;
c) a heavy chain comprising the amino acid sequence of SEQ ID NO: 426 and a light chain comprising the amino acid sequence of SEQ ID NO: 427; or
d) a heavy chain comprising the amino acid sequence of SEQ ID NO: 428 and a light chain comprising the amino acid sequence of SEQ ID NO: 429.

22. The method of claim 21, wherein the anti-hPD-1 antibody, or antigen-binding fragment thereof, portion of the immunoconjugate comprises a heavy chain comprising the amino acid sequence of SEQ ID NO: 414 and a light chain comprising the amino acid sequence of SEQ ID NO: 415.

23. The method of claim 1, wherein the immunoconjugate comprises:
a light chain comprising the amino acid sequence of SEQ ID NO: 415; and
a heavy chain-modified hIL-2 protein fusion comprising the amino acid sequence of SEQ ID NO: 532.

24. The method of claim 1, wherein the antagonistic anti-PD-1 antibody and the immunoconjugate are administered to the subject together in a mixture, concurrently as single agents, or sequentially as single agents in any order.

25. The method of claim 24, comprising:
administering the immunoconjugate prior to administering the antagonistic anti-PD-1 antibody;
administering the antagonistic anti-PD-1 antibody prior to administering the immunoconjugate; or
administering the immunoconjugate at substantially the same time as administering the antagonistic anti-PD-1 antibody.

26. The method of claim 1, wherein the cancer is melanoma, Merkel cell carcinoma, non-small cell lung carcinoma, renal cell carcinoma, triple negative breast cancer, squamous cell carcinoma of the head/neck, hepatocellular carcinoma, or microsatellite instability-high tumors or tumors with deficient DNA mismatch repair.

27. A method of treating a renal cell carcinoma, a triple negative breast cancer, a squamous cell carcinoma of the head/neck, a Merkel cell carcinoma, a hepatocellular carcinoma, or a microsatellite instability-high tumor or tumor with deficient DNA mismatch repair in a subject, the method comprising administering to the subject:
an anti-human PD-1 (hPD-1) antibody-modified human interleukin-2 (hIL-2) immunoconjugate comprising:
a modified hIL-2 protein comprising a D20A, D20S, D20Q, D20M, D20I, D20V, D20N, D20G, D20T, or D20E substitution at amino acid position 20 relative to the non-modified hIL-2 amino acid sequence of SEQ ID NO: 345 and an R38E substitution at amino acid position 38 relative to the non-modified hIL-2 amino acid sequence of SEQ ID NO: 345; and
an anti-hPD-1 antibody, or antigen-binding fragment thereof, that immunospecifically binds to hPD-1, wherein the antibody or antigen-binding fragment thereof comprises:
(i) a heavy chain complementarity determining region 1 (CDR1) comprising the amino acid sequence of SEQ ID NO: 418, a heavy chain CDR2 comprising the amino acid sequence of SEQ ID NO: 419, a heavy chain CDR3 comprising the amino acid sequence of SEQ ID NO: 420, a light chain CDR1 comprising the amino acid sequence of SEQ ID NO: 421, a light chain CDR2 comprising the amino acid sequence of SEQ ID NO: 422, and a light chain CDR3 comprising the amino acid sequence of SEQ ID NO: 423;
(ii) a heavy chain CDR1 comprising the amino acid sequence of SEQ ID NO: 386, a heavy chain CDR2 comprising the amino acid sequence of SEQ ID NO: 387, a heavy chain CDR3 comprising the amino acid sequence of SEQ ID NO: 388, a light chain CDR1 comprising the amino acid sequence of SEQ ID NO: 389, a light chain CDR2 comprising the amino acid sequence of SEQ ID NO: 390, and a light chain CDR3 comprising the amino acid sequence of SEQ ID NO: 391;
(iii) a heavy chain CDR1 comprising the amino acid sequence of SEQ ID NO: 396, a heavy chain CDR2 comprising the amino acid sequence of SEQ ID NO: 397, a heavy chain CDR3 comprising the amino acid sequence of SEQ ID NO: 398, a light chain CDR1 comprising the amino acid sequence of SEQ ID NO: 399, a light chain CDR2 comprising the amino acid sequence of SEQ ID NO: 400, and a light chain CDR3 comprising the amino acid sequence of SEQ ID NO: 401; or
(iv) a heavy chain CDR1 comprising the amino acid sequence of SEQ ID NO: 406, a heavy chain CDR2 comprising the amino acid sequence of SEQ ID NO: 407, a heavy chain CDR3 comprising the amino acid sequence of SEQ ID NO: 408, a light chain CDR1 comprising the amino acid sequence of SEQ ID NO: 409, a light chain CDR2 comprising the amino acid sequence of SEQ ID NO: 410, and a light chain CDR3 comprising the amino acid sequence of SEQ ID NO: 411.

28. The method of claim 27, wherein the modified hIL-2 protein comprises the amino acid sequence of any one of SEQ ID NOs: 149, 307, 607-611, 614, 617, or 620.

29. The method of claim 27, wherein the modified hIL-2 protein comprises a D20A substitution relative to the non-modified hIL-2 amino acid sequence of SEQ ID NO: 345 and a R38E substitution relative to the non-modified hIL-2 amino acid sequence of SEQ ID NO: 345.

30. The method of claim 29, wherein the modified hIL-2 protein comprises the amino acid sequence of SEQ ID NO: 149.

31. The method of claim 27, wherein the modified hIL-2 protein further comprises a deletion or substitution at amino acid position 3 relative to the non-modified hIL-2 amino acid sequence of SEQ ID NO: 345.

32. The method of claim 31, wherein the substitution at amino acid position 3 of the modified hIL-2 protein is T3A.

33. The method of claim 32, wherein the modified hIL-2 protein comprises the amino acid sequence of SEQ ID NO: 216.

34. The method of claim 31, wherein the modified hIL-2 protein comprises the amino acid sequence of SEQ ID NO: 218.

35. The method of claim 27, wherein the modified hIL-2 protein further comprises a deletion or substitution at amino acid position 125 relative to the non-modified hIL-2 amino acid sequence of SEQ ID NO: 345.

36. The method of claim 35, wherein the substitution at amino acid position 125 is C125A.

37. The method of claim 36, wherein the modified hIL-2 protein comprises the amino acid sequence of SEQ ID NO: 215, 217, or 219.

38. The method of claim 37, wherein the modified hIL-2 protein comprises the amino acid sequence of SEQ ID NO: 217.

39. The method of claim 27, wherein the modified hIL-2 protein is fused to the anti-hPD-1 antibody, or antigen-binding fragment thereof, portion of the immunoconjugate at the N-terminus of an antibody light chain, the C-terminus of an antibody light chain, the N-terminus of an antibody heavy chain, the C-terminus of an antibody heavy chain, the N-terminus of the antigen-binding fragment, or the C-terminus of the antigen-binding fragment.

40. The method of claim 27, wherein the modified hIL-2 protein is directly fused by a peptide bond to the anti-hPD-1 antibody, or antigen-binding fragment thereof, portion of the immunoconjugate.

41. The method of claim 40, wherein the modified hIL-2 protein is directly fused by a peptide bond to the C-terminal amino acid residue of the anti-hPD-1 antibody, or antigen-binding fragment thereof, portion of the immunoconjugate.

42. The method of claim 27, wherein the modified hIL-2 protein is fused to the anti-hPD-1 antibody, or antigen-binding fragment thereof, portion of the immunoconjugate through a linker.

43. The method of claim 27, wherein the anti-hPD-1 antibody, or antigen-binding fragment thereof, portion of the immunoconjugate comprises:
a) a heavy chain variable region comprising the amino acid sequence of SEQ ID NO: 416 and a light chain variable region comprising the amino acid sequence of SEQ ID NO: 417;
b) a heavy chain variable region comprising the amino acid sequence of SEQ ID NO: 384 and a light chain variable region comprising the amino acid sequence of SEQ ID NO: 385;
c) a heavy chain variable region comprising the amino acid sequence of SEQ ID NO: 394 and a light chain variable region comprising the amino acid sequence of SEQ ID NO: 395; or
d) a heavy chain variable region comprising the amino acid sequence of SEQ ID NO: 404 and a light chain variable region comprising the amino acid sequence of SEQ ID NO: 405.

44. The method of claim 27, wherein the anti-hPD-1 antibody, or antigen-binding fragment thereof, portion of the immunoconjugate comprises an IgG1 heavy chain constant region.

45. The method of claim 44, wherein the anti-hPD-1 antibody, or antigen-binding fragment thereof, portion of the immunoconjugate comprises an L235A substitution and a G237A substitution, according to EU numbering.

46. The method of claim 27, wherein the anti-hPD-1 antibody, or antigen-binding fragment thereof, portion of the immunoconjugate comprises:
a) a heavy chain comprising the amino acid sequence of SEQ ID NO: 414 and a light chain comprising the amino acid sequence of SEQ ID NO: 415;
b) a heavy chain comprising the amino acid sequence of SEQ ID NO: 424 and a light chain comprising the amino acid sequence of SEQ ID NO: 425;
c) a heavy chain comprising the amino acid sequence of SEQ ID NO: 426 and a light chain comprising the amino acid sequence of SEQ ID NO: 427; or
d) a heavy chain comprising the amino acid sequence of SEQ ID NO: 428 and a light chain comprising the amino acid sequence of SEQ ID NO: 429.

47. The method of claim 46, wherein the anti-hPD-1 antibody, or antigen-binding fragment thereof, portion of the immunoconjugate comprises a heavy chain comprising the amino acid sequence of SEQ ID NO: 414 and a light chain comprising the amino acid sequence of SEQ ID NO: 415.

48. The method of claim 27, wherein the immunoconjugate comprises:
a light chain comprising the amino acid sequence of SEQ ID NO: 415; and
a heavy chain-modified hIL-2 protein fusion comprising the amino acid sequence of SEQ ID NO: 532.
